(19) United States
(12) Patent Application Publication
(10) Pub. No.: US 2024/0110230 A1
(43) Pub. Date: Apr. 4, 2024

(54) BIOMARKERS FOR CANCER TREATMENT

(71) Applicant: Foundation Medicine, Inc., Cambridge, MA (US)

(72) Inventor: Jeffrey ROSS, Lebanon Springs, NY (US)

(73) Assignee: Foundation Medicine, Inc., Cambridge, MA (US)

(21) Appl. No.: 18/276,223

(22) PCT Filed: Feb. 9, 2022
(86) PCT No.: PCT/US2022/070591

§ 371 Date: Aug. 7, 2023

Related U.S. Application Data
(60) Provisional application No. 63/189,025, filed on May 14, 2021.
(60) Provisional application No. 63/148,116, filed on Feb. 10, 2021.

(51) Int. Cl. C12Q 1/6827; A61K 45/06; A61P 35/00

(52) U.S. Cl. CPC C12Q 1/6827; A61K 45/06; A61P 35/00; G01N 2800/52

(57) ABSTRACT

The present disclosure provides biomarkers associated with cancers, such as urothelial bladder cancer, non-small cell lung cancer, and renal cell carcinoma, as well as methods, kits and reagents for detecting such biomarkers. The disclosure also provides methods for evaluating, identifying, assessing, and/or treating an individual having a cancer, such as a urothelial bladder cancer, non-small cell lung cancer, and renal cell carcinoma.

### CROSS-REFERENCE TO RELATED APPLICATIONS

This application claims the benefit of U.S. Provisional Application No. 63/189,025, filed May 14, 2021, and U.S. Provisional Application No. 63/148,116, filed Feb. 10, 2021, the contents of each of which are hereby incorporated by reference in their entirety.

### FIELD

The present disclosure relates to biomarkers associated with diseases such as cancers, as well as to methods of diagnosis, assessment, and treatment of diseases such as cancers.

### BACKGROUND

Bladder cancer is one of the most common cancers of the urinary tract. Urothelial bladder cancer (UCB) is a common form of bladder cancer with high incidence worldwide. Current treatment options for UCB include chemotherapy and surgical interventions such as cystectomy and transurethral resection. However, treatment options are limited for patients with UCB that presents or progresses to chemorefractory metastatic disease.

Renal cell carcinoma (RCC) is the most common type of kidney cancer in adults. The cancer originates in the lining of the proximal convoluted tubule. Subtypes of RCC include clear cell renal cell carcinoma (ccRCC) and sarcomatoid renal cell carcinoma (srcRCC). ccRCC is the most common type of RCC. Current treatments for ccRCC include immunotherapy and certain targeted therapies. srcRCC is an aggressive variant of RCC that frequently presents as advanced-stage disease refractory to traditional systemic treatments for clear cell renal cell carcinoma (ccRCC).

Non-small cell lung cancer (NSCLC) is a type of cancer arising from tissues in the lung. Several sub-types of NSCLC exist, including adenocarcinoma, squamous cell carcinoma, large cell carcinoma, sarcomatoid carcinoma, salivary gland carcinoma, carcinoid tumor, unclassified carcinoma, adenosquamous carcinoma, or not otherwise specified. Smoking is a major risk factor for NSCLC, and most patients are diagnosed with advanced-stage disease. NSCLC is relatively difficult to treat and, when possible, surgical resection is used, especially at early stages of disease.

Cancer, such as UCB, srcRCC, ccRCC, and NSCLC, represents the phenotypic end-point of multiple genetic lesions that endow cells with a full range of biological properties required for tumorigenesis. Indeed, a hallmark genomic feature of many cancers is the presence of numerous complex chromosome structural aberrations, including translocations, intra-chromosomal inversions, point mutations, deletions, gene copy number changes, gene expression level changes, gene fusions, and germline mutations, among others.

Thus, there is a need in the art for characterizing the genomic landscape of UCB, srcRCC, ccRCC, and NSCLC to identify biomarkers and genomic lesions associated with such cancers, and for developing methods of identifying and evaluating patients with UCB, srcRCC, ccRCC, or NSCLC having such biomarkers or lesions. Such biomarkers or lesions can be an effective approach to develop compositions, methods and assays for evaluating and treating UCB, srcRCC, ccRCC, or NSCLC patients.

All references cited herein, including patents, patent applications and publications, are hereby incorporated by reference in their entirety. To the extent that any reference incorporated by reference conflicts with the instant disclosure, the instant disclosure shall control.

### BRIEF SUMMARY

In one aspect, provided herein is a method of identifying an individual having a urothelial bladder cancer (UCB) who may benefit from a treatment comprising an anti-cancer therapy, the method comprising detecting in a sample from the individual a deletion of a methylthioadenosine phosphorylase (MTAP) gene, or of a portion thereof, wherein the presence of the deletion of the MTAP gene, or of the portion thereof in the sample identifies the individual as one who may benefit from a treatment comprising an anti-cancer therapy.

In one aspect, provided herein is a method of identifying an individual having a urothelial bladder cancer (UCB) who may benefit from a treatment comprising a fibroblast growth factor receptor (FGFR)-targeted therapy or a phosphatase and tensin homolog (PTEN)-targeted therapy, the method comprising detecting in a sample from the individual a deletion of a methylthioadenosine phosphorylase (MTAP) gene, or of a portion thereof, and one or more mutations in a fibroblast growth factor receptor 3 (FGFR3) gene or in a PTEN gene, wherein: (a) the presence of the deletion of the MTAP gene, or of the portion thereof, and one or more mutations in the FGFR3 gene in the sample identifies the individual as one who may benefit from a treatment comprising an FGFR-targeted therapy; or (b) the presence of the deletion of the MTAP gene, or of the portion thereof, and one or more mutations in the PTEN gene in the sample identifies the individual as one who may benefit from a treatment comprising a PTEN-targeted therapy.

In one aspect, provided herein is a method of selecting a treatment for an individual having a UCB, the method comprising detecting a deletion of an MTAP gene, or of a portion thereof in a sample from the individual, wherein the presence of the deletion of the MTAP gene, or of the portion thereof, identifies the individual as one who may benefit from a treatment comprising an anti-cancer therapy.

In one aspect, provided herein is a method of selecting a treatment for an individual having a UCB, the method comprising detecting a deletion of an MTAP gene, or of a portion thereof, and one or more mutations in an FGFR3 gene or in a PTEN gene in a sample from the individual, wherein: (a) the presence of the deletion of the MTAP gene, or of the portion thereof, and one or more mutations in the FGFR3 gene in the sample identifies the individual as one who may benefit from a treatment comprising an FGFR-targeted therapy; or (b) the presence of the deletion of the MTAP gene, or of the portion thereof, and one or more mutations in the PTEN gene in the sample identifies the individual as one who may benefit from a treatment comprising a PTEN-targeted therapy.

In some embodiments, the presence in the sample of the deletion of the MTAP gene, or of the portion thereof, identifies the individual as one who may benefit from an additional treatment comprising a protein arginine methyltransferase 5 (PRMT5)-targeted therapy. In some embodiments, the presence in the sample of the deletion of the MTAP gene, or of the portion thereof, and the one or more mutations in the FGFR3 gene or in the PTEN gene identifies the individual as one who may benefit from an additional treatment comprising a protein arginine methyltransferase 5 (PRMT5)-targeted therapy.

In one aspect, provided herein is a method of identifying one or more treatment options for an individual having a UCB, the method comprising: (a) detecting a deletion of an MTAP gene, or of a portion thereof; and (b) generating a report, wherein the report comprises one or more treatment options identified for the individual based at least in part on the presence of the deletion of the MTAP gene, or of the portion thereof in the sample, wherein the one or more treatment options comprise an anti-cancer therapy.

In one aspect, provided herein is a method of identifying one or more treatment options for an individual having a UCB, the method comprising: (a) detecting a deletion of an MTAP gene, or of a portion thereof, and one or more mutations in an FGFR3 gene or in a PTEN gene in a sample from the individual; and (b) generating a report, wherein the report comprises: (i) one or more treatment options identified for the individual based at least in part on the presence of the deletion of the MTAP gene, or of the portion thereof, and one or more mutations in the FGFR3 gene in the sample, wherein the one or more treatment options comprise an FGFR-targeted therapy; or (ii) one or more treatment options identified for the individual based at least in part on the presence of the deletion of the MTAP gene, or of the portion thereof, and one or more mutations in the PTEN gene in the sample, wherein the one or more treatment options comprise a PTEN-targeted therapy.

In one aspect, provided herein is a method of identifying one or more treatment options for an individual having a UCB, the method comprising acquiring knowledge of a deletion of an MTAP gene, or of a portion thereof in a sample from the individual; and generating a report comprising one or more treatment options identified for the individual based at least in part on the knowledge of a deletion of the MTAP gene, or of a portion thereof, wherein the one or more treatment options comprise an anti-cancer therapy.

In one aspect, provided herein is a method of identifying one or more treatment options for an individual having a UCB, the method comprising acquiring knowledge of a deletion of an MTAP gene, or of a portion thereof, and one or more mutations in an FGFR3 gene or in a PTEN gene in a sample from the individual; and generating a report comprising: (a) one or more treatment options identified for the individual based at least in part on the knowledge of a deletion of the MTAP gene, or of a portion thereof, and one or more mutations in the FGFR3 gene in the sample, wherein the one or more treatment options comprise an FGFR-targeted therapy; or (b) one or more treatment options identified for the individual based at least in part on the knowledge of a deletion of the MTAP gene, or of a portion thereof, and one or more mutations in the PTEN gene in the sample, wherein the one or more treatment options comprise a PTEN-targeted therapy.

In some embodiments, the one or more treatment options identified for the individual further comprise a PRMT5-targeted therapy.

In one aspect, provided herein is a method of selecting a treatment for an individual having a UCB, comprising acquiring knowledge of a deletion of an MTAP gene, or of a portion thereof in a sample from the individual, wherein responsive to the acquisition of knowledge of a deletion of an MTAP gene, or of a portion thereof in the sample: (i) the individual is classified as a candidate to receive a treatment comprising an anti-cancer therapy; and/or (ii) the individual is identified as likely to respond to a treatment that comprises an anti-cancer therapy.

In one aspect, provided herein is a method of selecting a treatment for an individual having a UCB, comprising acquiring knowledge of a deletion of an MTAP gene, or of a portion thereof, and one or more mutations in an FGFR3 gene or in a PTEN gene in a sample from the individual, wherein: (a) responsive to the acquisition of knowledge of a deletion of an MTAP gene, or of a portion thereof, and one or more mutations in an FGFR3 gene in the sample: (i) the individual is classified as a candidate to receive a treatment comprising an FGFR-targeted therapy; and/or (ii) the individual is identified as likely to respond to a treatment that comprises an FGFR-targeted therapy; or (b) responsive to the acquisition of knowledge of a deletion of an MTAP gene, or of a portion thereof, and one or more mutations in a PTEN gene in the sample: (i) the individual is classified as a candidate to receive a treatment comprising a PTEN-targeted therapy; and/or (ii) the individual is identified as likely to respond to a treatment that comprises a PTEN-targeted therapy.

In some embodiments, the treatment further comprises a PRMT5-targeted therapy.

In one aspect, provided herein is a method of predicting survival of an individual having a UCB treated with a treatment comprising an anti-cancer therapy, the method comprising acquiring knowledge of a deletion of an MTAP gene, or of a portion thereof in a sample from the individual, wherein responsive to the acquisition of said knowledge, the individual is predicted to have longer survival after treatment with the anti-cancer therapy, as compared to an individual whose UCB does not exhibit a deletion of an MTAP gene, or of a portion thereof.

In one aspect, provided herein is a method of predicting survival of an individual having a UCB treated with a treatment comprising an FGFR-targeted therapy or a PTEN-targeted therapy, the method comprising acquiring knowledge of a deletion of an MTAP gene, or of a portion thereof, and one or more mutations in an FGFR3 gene or in a PTEN gene in a sample from the individual, wherein responsive to the acquisition of said knowledge, the individual is predicted to have longer survival after treatment with the FGFR-targeted therapy or the PTEN-targeted therapy, as compared to an individual whose UCB does not exhibit a deletion of an MTAP gene, or of a portion thereof, and one or more mutations in an FGFR3 gene or in a PTEN gene.

In one aspect, provided herein is a method of evaluating an individual having a UCB, suspected of having a UCB, being tested for a UCB, being treated for a UCB, or being tested for a susceptibility for UCB, comprising: acquiring genotype information that identifies a deletion of an MTAP gene, or of a portion thereof, in the individual, wherein said genotype information identifies the individual as: (i) having a UCB associated with the deletion of the MTAP gene, or of the portion thereof; or (ii) having an increased risk of UCB recurrence, aggressive UCB, anti-cancer therapy resistance, or poor prognosis.

In one aspect, provided herein is a method of evaluating an individual having a UCB, suspected of having a UCB, being tested for a UCB, being treated for a UCB, or being tested for a susceptibility for UCB, comprising: acquiring genotype information that identifies a deletion of an MTAP gene, or of a portion thereof, and one or more mutations in an FGFR3 gene or in a PTEN gene in the individual, wherein said genotype information identifies the individual as: (i) having a UCB associated with the deletion of the MTAP gene, or of the portion thereof, and the one or more mutations in the FGFR3 gene or in the PTEN gene; or (ii) having an increased risk of UCB recurrence, aggressive UCB, anti-cancer therapy resistance, or poor prognosis.

In one aspect, provided herein is a method of monitoring an individual having a UCB, comprising acquiring knowledge of a deletion of an MTAP gene, or of a portion thereof, in a sample from the individual, wherein responsive to the acquisition of said knowledge, the individual is predicted to have increased risk of UCB recurrence, aggressive UCB, anti-cancer therapy resistance, or poor prognosis, as compared to an individual whose UCB does not exhibit a deletion of an MTAP gene, or of a portion thereof, optionally wherein the individual is being treated for UCB.

In one aspect, provided herein is a method of monitoring an individual having a UCB, comprising acquiring knowledge of a deletion of an MTAP gene, or of a portion thereof, and one or more mutations in an FGFR3 gene or in a PTEN gene in a sample from the individual, wherein responsive to the acquisition of said knowledge, the individual is predicted to have increased risk of UCB recurrence, aggressive UCB, anti-cancer therapy resistance, or poor prognosis, as compared to an individual whose UCB does not exhibit a deletion of an MTAP gene, or of a portion thereof, and one or more mutations in an FGFR3 gene or in a PTEN gene, optionally wherein the individual is being treated for UCB.

In one aspect, provided herein is a method of screening an individual having a UCB, suspected of having a UCB, being tested for a UCB, being treated for a UCB, or being tested for a susceptibility for UCB, comprising acquiring knowledge of a deletion of an MTAP gene, or of a portion thereof, in a sample from the individual, wherein responsive to the acquisition of said knowledge, the individual is predicted to have increased risk of UCB recurrence, aggressive UCB, anti-cancer therapy resistance, or poor prognosis, as compared to an individual whose UCB does not exhibit a deletion of an MTAP gene, or of a portion thereof.

In one aspect, provided herein is a method of screening an individual having a UCB, suspected of having a UCB, being tested for a UCB, being treated for a UCB, or being tested for a susceptibility for UCB, comprising acquiring knowledge of a deletion of an MTAP gene, or of a portion thereof, and one or more mutations in an FGFR3 gene or in a PTEN gene in a sample from the individual, wherein responsive to the acquisition of said knowledge, the individual is predicted to have increased risk of UCB recurrence, aggressive UCB, anti-cancer therapy resistance, or poor prognosis, as compared to an individual whose UCB does not exhibit a deletion of an MTAP gene, or of a portion thereof, and one or more mutations in an FGFR3 gene or in a PTEN gene.

In one aspect, provided herein is a method of treating or delaying progression of a UCB, comprising acquiring knowledge of a deletion of an MTAP gene, or of a portion thereof, in a sample from an individual, and responsive to said knowledge, administering to the individual an effective amount of a treatment comprising an anti-cancer therapy.

In one aspect, provided herein is a method of treating or delaying progression of a UCB, comprising: (a) acquiring knowledge of a deletion of an MTAP gene, or of a portion thereof, and one or more mutations in an FGFR3 gene in a sample from an individual, and responsive to said knowledge, administering to the individual an effective amount of a treatment comprising an FGFR-targeted therapy; or (b) acquiring knowledge of a deletion of an MTAP gene, or of a portion thereof, and one or more mutations in a PTEN gene in a sample from an individual, and responsive to said knowledge, administering to the individual an effective amount of a treatment comprising a PTEN-targeted therapy.

In one aspect, provided herein is a method of treating or delaying progression of a UCB, comprising responsive to acquiring knowledge of a deletion of an MTAP gene, or of a portion thereof, administering to the individual an effective amount of a treatment comprising an anti-cancer therapy.

In one aspect, provided herein is a method of treating or delaying progression of a UCB, comprising: (a) responsive to acquiring knowledge of a deletion of an MTAP gene, or of a portion thereof, and one or more mutations in an FGFR3 gene in a sample from an individual, administering to the individual an effective amount of a treatment comprising an FGFR-targeted therapy; or (b) responsive to acquiring knowledge of a deletion of an MTAP gene, or of a portion thereof, and one or more mutations in a PTEN gene in a sample from an individual, administering to the individual an effective amount of a treatment comprising a PTEN-targeted therapy.

In some embodiments, the acquiring knowledge of the deletion of the MTAP gene, or of the portion thereof, comprises detecting the deletion of the MTAP gene, or of the portion thereof, in the sample. In some embodiments, the acquiring knowledge of the deletion of the MTAP gene, or of the portion thereof, and one or more mutations in the FGFR3 gene or in the PTEN gene comprises detecting the deletion of the MTAP gene, or of the portion thereof, and the one or more mutations in the FGFR3 gene or in the PTEN gene in the sample.

In one aspect, provided herein is a method of treating or delaying progression of a UCB, comprising detecting a deletion of an MTAP gene, or of a portion thereof, in a sample from an individual, and administering to the individual an effective amount of a treatment comprising an anti-cancer therapy.

In one aspect, provided herein is a method of treating or delaying progression of a UCB, comprising: (a) detecting a deletion of an MTAP gene, or of a portion thereof, and one or more mutations in an FGFR3 gene in a sample from an individual, and administering to the individual an effective amount of a treatment comprising an FGFR-targeted therapy; or (b) detecting a deletion of an MTAP gene, or of a portion thereof, and one or more mutations in a PTEN gene in a sample from an individual, and administering to the individual an effective amount of a treatment comprising a PTEN-targeted therapy.

In one aspect, provided herein is a method of detecting the presence or absence of a UCB in an individual, comprising: (a) detecting the presence or absence of a UCB in a sample from an individual; and (i) detecting the presence or absence of a deletion of an MTAP gene, or of a portion thereof, and one or more mutations in an FGFR3 gene in the sample, or (ii) detecting the presence or absence of a deletion of an MTAP gene, or of a portion thereof, and one or more mutations in a PTEN gene in the sample.

In one aspect, provided herein is a method of assessing a UCB in an individual, the method comprising: (a) detecting a deletion of an MTAP gene, or of a portion thereof, in a sample from the individual; and (b) providing an assessment of the deletion of the MTAP gene, or of the portion thereof.

In one aspect, provided herein is a method of assessing a UCB in an individual, the method comprising: (a) detecting a deletion of an MTAP gene, or of a portion thereof, and one or more mutations in an FGFR3 gene or in a PTEN gene in a sample from the individual; and (b) providing an assessment of the deletion of the MTAP gene, or of the portion thereof, and the one or more mutations in the FGFR3 gene or in the PTEN gene.

In one aspect, provided herein is a method of detecting a deletion of an MTAP gene, or of a portion thereof, the method comprising detecting the deletion of the MTAP gene, or of the portion thereof, in a sample from an individual having a UCB.

In one aspect, provided herein is a method of detecting a deletion of an MTAP gene, or of a portion thereof, and one or more mutations in an FGFR3 gene or in a PTEN gene, the method comprising detecting the deletion of the MTAP gene, or of the portion thereof, and the one or more mutations in the FGFR3 gene or in the PTEN gene in a sample from an individual having a UCB.

In one aspect, provided herein is a method of detecting a deletion of an MTAP gene, or of a portion thereof, the method comprising: (a) providing a sample from an individual having a UCB, wherein the sample comprises one or more nucleic acids; (b) preparing a nucleic acid sequencing library from the one or more nucleic acids in the sample; (c) amplifying said library using a polymerase chain reaction (PCR); (d) selectively enriching for one or more nucleic acids comprising MTAP gene nucleotide sequences in said library to produce an enriched sample; (e) sequencing the enriched sample, thereby producing a plurality of sequencing reads; (f) analyzing the plurality of sequencing reads for the presence of a deletion of an MTAP gene, or of a portion thereof; (g) detecting, based on the analyzing step, a deletion of an MTAP gene, or of a portion thereof, in the sample from the individual.

In one aspect, provided herein is a method of detecting a deletion of an MTAP gene, or of a portion thereof, and one or more mutations in an FGFR3 gene or in a PTEN gene, the method comprising: (a) providing a sample from an individual having a UCB, wherein the sample comprises one or more nucleic acids; (b) preparing a nucleic acid sequencing library from the one or more nucleic acids in the sample; (c) amplifying said library using a polymerase chain reaction (PCR); (d) selectively enriching for one or more nucleic acids comprising MTAP, FGFR3, or PTEN gene nucleotide sequences in said library to produce an enriched sample; (e) sequencing the enriched sample, thereby producing a plurality of sequencing reads; (f) analyzing the plurality of sequencing reads for the presence of a deletion of an MTAP gene, or of a portion thereof, and one or more mutations in an FGFR3 gene or in a PTEN gene; (g) detecting, based on the analyzing step, a deletion of an MTAP gene, or of a portion thereof, and one or more mutations in an FGFR3 gene or in a PTEN gene in the sample from the individual.

In some embodiments, the methods further comprise selectively enriching for one or more of: (a) one or more nucleic acids comprising nucleotide sequences that comprise the deletion of the MTAP gene, or of the portion thereof, (b) one or more nucleic acids comprising nucleotide sequences that comprise the one or more mutations in the FGFR3 gene, or (c) one or more nucleic acids comprising nucleotide sequences that comprise the one or more mutations in the PTEN gene; wherein the selectively enriching produces an enriched sample.

In one aspect, provided herein is a method of treating or delaying progression of a UCB, comprising administering to an individual having a UCB an effective amount of a treatment comprising an anti-cancer therapy, wherein the UCB comprises a deletion of an MTAP gene, or of a portion thereof.

In one aspect, provided herein is a method of treating or delaying progression of a UCB, comprising: (a) administering to an individual having a UCB an effective amount of a treatment comprising an FGFR-targeted therapy, wherein the UCB comprises a deletion of an MTAP gene, or of a portion thereof, and one or more mutations in an FGFR3 gene; or (b) administering to an individual having a UCB an effective amount of a treatment comprising a PTEN-targeted therapy, wherein the UCB comprises a deletion of an MTAP gene, or of a portion thereof, and one or more mutations in a PTEN gene.

In some embodiments, the methods further comprise acquiring knowledge of or detecting in the sample a deletion of a CDKN2A gene, or of a portion thereof; and/or a deletion of a CDKN2B gene, or of a portion thereof. In some embodiments, the methods comprise acquiring knowledge of or detecting in the sample one or more mutations in one or more genes, wherein the one or more genes comprise TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the individual has one or more mutations in one or more genes, wherein the one or more genes comprise TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1.

In some embodiments, the methods further comprise administering to the individual an effective amount of a treatment comprising an anti-cancer therapy. In some embodiments, the methods further comprise selectively enriching for one or more nucleic acids comprising nucleotide sequences that comprise one or more mutations in one or more genes, wherein the one or more genes comprise TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, to produce an enriched sample. In some embodiments, the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the individual has a deletion of a CDKN2A gene, or of a portion thereof; and/or a deletion of a CDKN2B gene, or of a portion thereof. In some embodiments, the individual has a heterozygous or a homozygous deletion of a CDKN2A gene, or of a portion thereof; and/or a heterozygous or a homozygous deletion of a CDKN2B gene, or of a portion thereof. In some embodiments, the individual has a heterozygous or a homozygous deletion of the MTAP gene, or of a portion thereof. In some embodiments, the methods further comprise administering to the individual a PRMT5-targeted therapy. In some embodiments, the individual is being treated with a PRMT5-targeted therapy. In some embodiments, the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437. In some embodiments, the one or more mutations in the FGFR3 gene or the PTEN gene comprise one or more of an insertion, deletion or substitution of one or more nucleotides, a genomic rearrangement, an alteration in a promoter, a gene fusion, or a copy number alteration. In some embodiments, the one or more mutations in the FGFR3 gene or the PTEN gene result in one or more of an insertion, deletion or substitution of one or more amino acid residues in a polypeptide encoded by the FGFR3 gene or the PTEN gene. In some embodiments, the FGFR-targeted therapy or the PTEN-targeted therapy is a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the FGFR-targeted therapy comprises one or more of: a multi-kinase inhibitor, an FGFR-selective inhibitor, an FGFR3-specific inhibitor, or a combination therapy. In some embodiments, the multi-kinase inhibitor comprises one or more of E3810 (lucitanib), AZD4547, Dovitinib (TK1258), Ponatinib, Derazantinib (ARQ 087), Nintendanib (BIBF1120), Rogaratinib (BAY 1163877), 3D185, SOMCL-085, brivanib (BMS582664), lenvatinib (E7080), or orantinib (TSU-68). In some embodiments, the FGFR-selective inhibitor comprises one or more of PRN1371, XL-228, AZ12908010 (AZ8010), Debio-1347 (CH5183284), FIIN-2, LY2874455, Infigratinib (BGJ398, NVP-BGJ398), Pemigatinib (Pemazyre®, INCB054828), Erdafitinib (JNJ-42756493, Balversa®), ASP5878, TAS-120, PRN1371, or PKC412. In some embodiments, the FGFR3-specific inhibitor comprises one or more of Vofatamab or MFGR1877S. In some embodiments, the combination therapy comprises one or more of: an FGFR-targeted therapy and a PD-1 or a PD-L1-targeted therapy; an FGFR-targeted therapy and an EGFR inhibitor; an FGFR-targeted therapy and an immunotherapy; an FGFR-targeted therapy and a MAPK inhibitor; an FGFR-targeted therapy and a PI3K inhibitor; an FGFR-targeted therapy and an AKT inhibitor; or an FGFR-targeted therapy and a VEGF inhibitor. In some embodiments, the PTEN-targeted therapy comprises one or more of: a PI3K inhibitor, an AKT inhibitor, an mTOR inhibitor, or a MET inhibitor. In some embodiments, the deletion of the MTAP gene or of the portion thereof, or the one or more mutations in the FGFR3 gene or the PTEN gene are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the deletion of the MTAP gene or of the portion thereof, or the one or more mutations in the FGFR3 gene or the PTEN gene are detected in a polypeptide encoded by the MTAP gene, the FGFR3 gene, or the PTEN gene. In some embodiments, the deletion of the MTAP gene or of the portion thereof, or the one or more mutations in the FGFR3 gene or the PTEN gene are detected in the sample by one or more of: immunoblotting, enzyme linked immunosorbent assay (ELISA), immunohistochemistry, or mass spectrometry. In some embodiments, the deletion of the CDKN2A gene or of the portion thereof, and/or the deletion of the CDKN2B gene or of the portion thereof, are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the deletion of the CDKN2A gene or of the portion thereof, and/or the deletion of the CDKN2B gene or of the portion thereof, are detected in a polypeptide encoded by the CDKN2A gene or the CDKN2B gene. In some embodiments, the deletion of the CDKN2A gene or of the portion thereof, and/or the deletion of the CDKN2B gene or of the portion thereof, are detected in the sample by one or more of: immunoblotting, enzyme linked immunosorbent assay (ELISA), immunohistochemistry, or mass spectrometry.

In some embodiments, the UCB is chemorefractory UCB. In some embodiments, the UCB is metastatic UCB. In some embodiments, the treatment or the one or more treatment options further comprise an additional anti-cancer treatment. In some embodiments, the additional anti-cancer treatment comprises one or more of a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the additional anti-cancer treatment comprises one or more of a surgery, radiotherapy, chemotherapy, anti-angiogenic therapy, anti-DNA repair therapy, anti-inflammatory therapy, an anti-neoplastic agent, a chemotherapeutic agent, a growth inhibitory agent, an anti-angiogenic agent, or a cytotoxic agent. In some embodiments, the sample from the individual comprises fluid, cells, or tissue. In some embodiments, the sample from the individual comprises a tumor biopsy or a circulating tumor cell. In some embodiments, the sample from the individual comprises one or more nucleic acids. In some embodiments, the sample from the individual comprises mRNA, genomic DNA, circulating tumor DNA, cell-free DNA, or cell-free RNA. In some embodiments, the sample from the individual comprises one or more polypeptides. In some embodiments, the sample is a formalin-fixed paraffin-embedded (FFPE) sample. In some embodiments, the sample comprises one or more nucleic acids and/or one or more polypeptides obtained from an FFPE sample from the individual. In some embodiments, the one or more nucleic acids comprise mRNA and/or genomic DNA. In some embodiments, the sample is derived from a UCB in the individual. In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the bait comprises a capture nucleic acid molecule configured to hybridize to the one or more nucleic acids. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and 200 nucleotides. In some embodiments, the bait is conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample.

In one aspect, provided herein is a method of identifying an individual having a urothelial bladder cancer (UCB) who may benefit from a treatment comprising an immunotherapy or a retinoblastoma (RB1)-targeted therapy, the method comprising detecting in a sample from the individual one or more of: (a) a tumor mutational burden (TMB) of at least about 10 mut/Mb or at least about 20 mut/Mb, (b) a PD-L1-positive UCB, or (c) one or more mutations in an RB1 gene; wherein: (i) the presence in the sample of a TMB of at least about 10 mut/Mb or at least about 20 mut/Mb, or of a PD-L1-positive UCB identifies the individual as one who may benefit from a treatment comprising an immunotherapy; or (ii) the presence in the sample of one or more mutations in the RB1 gene identifies the individual as one who may benefit from a treatment comprising an RB1-targeted therapy.

In one aspect, provided herein is a method of selecting a treatment for an individual having a UCB, the method comprising detecting one or more of: (a) a TMB of at least about 10 mut/Mb or at least about 20 mut/Mb, (b) a PD-L1-positive UCB, or (c) one or more mutations in an RB1 gene, in a sample from the individual; wherein: (i) the presence in the sample of a TMB of at least about 10 mut/Mb or at least about 20 mut/Mb, or of a PD-L1-positive UCB identifies the individual as one who may benefit from a treatment comprising an immunotherapy; or (ii) the presence in the sample of one or more mutations in the RB1 gene identifies the individual as one who may benefit from a treatment comprising an RB1-targeted therapy.

In one aspect, provided herein is a method of identifying one or more treatment options for an individual having a UCB, the method comprising: (a) detecting in a sample from the individual one or more of: (i) a TMB of at least about 10 mut/Mb or at least about 20 mut/Mb, (ii) a PD-L1-positive UCB, or (iii) one or more mutations in an RB1 gene; and (b) generating a report, wherein the report comprises: (i) one or more treatment options identified for the individual based at least in part on the presence of a TMB of at least about 10 mut/Mb or at least about 20 mut/Mb, or of a PD-L1-positive UCB in the sample, wherein the one or more treatment options comprise an immunotherapy; or (ii) one or more treatment options identified for the individual based at least in part on the presence of one or more mutations in the RB1 gene in the sample, wherein the one or more treatment options comprise an RB1-targeted therapy.

In one aspect, provided herein is a method of identifying one or more treatment options for an individual having a UCB, the method comprising acquiring knowledge of one or more of: (a) a TMB of at least about 10 mut/Mb or at least about 20 mut/Mb, (b) a PD-L1-positive UCB, or (c) one or more mutations in an RB1 gene, in a sample from the individual; and generating a report comprising: (i) one or more treatment options identified for the individual based at least in part on the knowledge of a TMB of at least about 10 mut/Mb or at least about 20 mut/Mb, or of a PD-L1-positive UCB in the sample, wherein the one or more treatment options comprise an immunotherapy; or (ii) one or more treatment options identified for the individual based at least in part on the knowledge of one or more mutations in the RB1 gene in the sample, wherein the one or more treatment options comprise an RB1-targeted therapy.

In one aspect, provided herein is a method of selecting a treatment for an individual having a UCB, comprising acquiring knowledge of one or more of: (a) a TMB of at least about 10 mut/Mb or at least about 20 mut/Mb, (b) a PD-L1-positive UCB, or (c) one or more mutations in an RB1 gene, in a sample from the individual, wherein: (a) responsive to acquisition of knowledge of a TMB of at least about 10 mut/Mb or at least about 20 mut/Mb, or of a PD-L1-positive UCB in the sample: (i) the individual is classified as a candidate to receive a treatment comprising an immunotherapy; and/or (ii) the individual is identified as likely to respond to a treatment that comprises an immunotherapy; or (b) responsive to acquisition of knowledge of one or more mutations in the RB1 gene in the sample: (i) the individual is classified as a candidate to receive a treatment comprising an RB1-targeted therapy; and/or (ii) the individual is identified as likely to respond to a treatment that comprises an RB1-targeted therapy.

In one aspect, provided herein is a method of predicting survival of an individual having UCB treated with a treatment comprising an immunotherapy or an RB1-targeted therapy, the method comprising acquiring knowledge of one or more of: (a) a TMB of at least about 10 mut/Mb or at least about 20 mut/Mb, (b) a PD-L1-positive UCB, or (c) one or more mutations in an RB1 gene, in a sample from the individual; wherein responsive to acquisition of said knowledge, the individual is predicted to have longer survival after treatment with the immunotherapy or the RB1-targeted therapy, as compared to an individual whose UCB does not exhibit a TMB of at least about 10 mut/Mb or at least about 20 mut/Mb, a PD-L1-positive UCB, or one or more mutations in the RB1 gene.

In one aspect, provided herein is a method of evaluating an individual having a UCB, suspected of having a UCB, being tested for a UCB, being treated for a UCB, or being tested for a susceptibility for UCB, comprising: acquiring genotype information that identifies one or more of: (a) a TMB of at least about 10 mut/Mb or at least about 20 mut/Mb, (b) a PD-L1-positive UCB, or (c) one or more mutations in an RB1 gene, in the individual, wherein said genotype information identifies the individual as: (i) having a UCB associated with the TMB of at least about 10 mut/Mb or at least about 20 mut/Mb, the PD-L1-positive UCB, or the one or more mutations in the RB1 gene; or (ii) having an increased risk of UCB recurrence, aggressive UCB, anti-cancer therapy resistance, or poor prognosis.

In one aspect, provided herein is a method of monitoring an individual having a UCB, comprising acquiring knowledge of one or more of: (a) a TMB of at least about 10 mut/Mb or at least about 20 mut/Mb, (b) a PD-L1-positive UCB, or (c) one or more mutations in an RB1 gene, in a sample from the individual, wherein responsive to acquisition of said knowledge, the individual is predicted to have increased risk of UCB recurrence, aggressive UCB, anti-cancer therapy resistance, or poor prognosis, as compared to an individual whose UCB does not exhibit a TMB of at least about 10 mut/Mb or at least about 20 mut/Mb, a PD-L1-positive UCB, or one or more mutations in the RB1 gene, optionally wherein the individual is being treated for UCB.

In one aspect, provided herein is a method of screening an individual having a UCB, suspected of having a UCB, being tested for a UCB, being treated for a UCB, or being tested for a susceptibility for UCB, comprising acquiring knowledge of one or more of: (a) a TMB of at least about 10 mut/Mb or at least about 20 mut/Mb, (b) a PD-L1-positive UCB, or (c) one or more mutations in an RB1 gene, in a sample from the individual; wherein responsive to acquisition of said knowledge, the individual is predicted to have increased risk of UCB recurrence, aggressive UCB, anti-cancer therapy resistance, or poor prognosis, as compared to an individual whose UCB does not exhibit a TMB of at least about 10 mut/Mb or at least about 20 mut/Mb, a PD-L1-positive UCB, or one or more mutations in the RB1 gene.

In one aspect, provided herein is a method of treating or delaying progression of a UCB, comprising: (a) acquiring knowledge of a TMB of at least about 10 mut/Mb or at least about 20 mut/Mb, or a PD-L1-positive UCB in a sample from an individual, and responsive to said knowledge, administering to the individual an effective amount of a treatment comprising an immunotherapy; or (b) acquiring knowledge of one or more mutations in an RB1 gene in a sample from an individual, and responsive to said knowledge, administering to the individual an effective amount of a treatment comprising an RB1-targeted therapy.

In one aspect, provided herein is a method of treating or delaying progression of a UCB, comprising: (a) responsive to acquiring knowledge of a TMB of at least about 10 mut/Mb or at least about 20 mut/Mb, or a PD-L1-positive UCB in a sample from an individual, administering to the individual an effective amount of a treatment comprising an immunotherapy; or (b) responsive to acquiring knowledge of one or more mutations in an RB1 gene in a sample from an individual, administering to the individual an effective amount of a treatment comprising an RB1-targeted therapy.

In some embodiments, acquiring knowledge of a TMB of at least about 10 mut/Mb or at least about 20 mut/Mb, a PD-L1-positive UCB, or one or more mutations in an RB1 gene comprises detecting the TMB of at least about 10 mut/Mb or at least about 20 mut/Mb, the PD-L1-positive UCB, or the one or more mutations in the RB1 gene in the sample.

In one aspect, provided herein is a method of treating or delaying progression of a UCB, comprising: (a) detecting a TMB of at least about 10 mut/Mb or at least about 20 mut/Mb, or a PD-L1-positive UCB in a sample from an individual, and administering to the individual an effective amount of a treatment comprising an immunotherapy; or (b) detecting one or more mutations in an RB1 gene in a sample from an individual, and administering to the individual an effective amount of a treatment comprising an RB1-targeted therapy.

In one aspect, provided herein is a method of detecting the presence or absence of a UCB in an individual, comprising: (a) detecting the presence or absence of a UCB in a sample from an individual; and (i) detecting the presence or absence of a TMB of at least about 10 mut/Mb or at least about 20 mut/Mb, or a PD-L1-positive UCB in the sample, or (ii) detecting the presence or absence of one or more mutations in an RB1 gene in the sample.

In one aspect, provided herein is a method of assessing a UCB in an individual, the method comprising: (a) detecting one or more of: (i) a TMB of at least about 10 mut/Mb or at least about 20 mut/Mb, (ii) a PD-L1-positive UCB, or (iii) one or more mutations in an RB1 gene, in a sample from the individual; and (b) providing an assessment of the TMB of at least about 10 mut/Mb or at least about 20 mut/Mb, the PD-L1-positive UCB, or the one or more mutations in the RB1 gene in the sample.

In one aspect, provided herein is a method of detecting a TMB of at least about 10 mut/Mb or at least about 20 mut/Mb, a PD-L1-positive UCB, or one or more mutations in an RB1 gene, the method comprising detecting the TMB of at least about 10 mut/Mb or at least about 20 mut/Mb, the PD-L1-positive UCB, or the one or more mutations in the RB1 gene in a sample from an individual having a UCB.

In one aspect, provided herein is a method of detecting one or more mutations in an RB1 gene, the method comprising: (a) providing a sample from an individual having a UCB, wherein the sample comprises one or more nucleic acids; (b) preparing a nucleic acid sequencing library from the one or more nucleic acids in the sample; (c) amplifying said library using a polymerase chain reaction (PCR); (d) selectively enriching for one or more nucleic acids comprising RB1 gene nucleotide sequences in said library to produce an enriched sample; (e) sequencing the enriched sample, thereby producing a plurality of sequencing reads; (f) analyzing the plurality of sequencing reads for the presence of one or more mutations in an RB1 gene; (g) detecting, based on the analyzing step, one or more mutations in an RB1 gene in the sample from the individual.

In one aspect, provided herein is a method of detecting a TMB of at least about 10 mut/Mb or at least about 20 mut/Mb, the method comprising: (a) providing a sample from an individual having a UCB, wherein the sample comprises one or more nucleic acids; (b) preparing a nucleic acid sequencing library from the one or more nucleic acids in the sample; (c) amplifying said library using a polymerase chain reaction (PCR); (d) sequencing said library, thereby producing a plurality of sequencing reads; (e) analyzing the plurality of sequencing reads for the presence of a TMB of at least about 10 mut/Mb or at least about 20 mut/Mb; (f) detecting, based on the analyzing step, a TMB of at least about 10 mut/Mb or at least about 20 mut/Mb in the sample from the individual.

In one aspect, provided herein is a method of treating or delaying progression of a UCB, comprising: (a) administering to an individual having a UCB an effective amount of a treatment comprising an immunotherapy, wherein the UCB comprises a TMB of at least about 10 mut/Mb or at least about 20 mut/Mb, or wherein the UCB is PD-L1-positive; or (b) administering to an individual having a UCB an effective amount of a treatment comprising an RB1-targeted therapy, wherein the UCB comprises one or more mutations in an RB1 gene.

In some embodiments, the methods further comprise acquiring knowledge of the absence of a deletion of an MTAP gene, or of a portion thereof, in the sample. In some embodiments, acquiring knowledge of the absence of a deletion of an MTAP gene, or of a portion thereof, comprises detecting the absence of a deletion of an MTAP gene, or of a portion thereof, in the sample. In some embodiments, the methods further comprise detecting the absence of a deletion of an MTAP gene, or of a portion thereof, in the sample. In some embodiments, the methods comprise acquiring knowledge of or detecting in the sample one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the individual has one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the methods further comprise administering to the individual an effective amount of a treatment comprising anti-cancer therapy. In some embodiments, the methods comprise selectively enriching for one or more nucleic acids comprising nucleotide sequences that comprise one or more mutations in one or more genes to produce an enriched sample, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the one or more mutations in the one or more genes are detected in a polypeptide encoded by the one or more genes. In some embodiments, the one or more mutations in the one or more genes are detected in the sample by one or more of immunoblotting, enzyme linked immunosorbent assay (ELISA), immunohistochemistry, or mass spectrometry. In some embodiments, the methods further comprise selectively enriching for one or more nucleic acids comprising nucleotide sequences that comprise a deletion of the MTAP gene, or of a portion thereof, to produce an enriched sample. In some embodiments, the absence of a deletion of an MTAP gene, or of a portion thereof, is detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the individual does not have a deletion of an MTAP gene, or of a portion thereof. In some embodiments, the methods further comprise selectively enriching for one or more nucleic acids comprising nucleotide sequences that comprise the one or more mutations in the RB1 gene to produce an enriched sample. In some embodiments, the one or more mutations in the RB1 gene comprise one or more of an insertion, deletion or substitution of one or more nucleotides, a genomic rearrangement, an alteration in a promoter of the RB1 gene, a gene fusion, or a copy number alteration. In some embodiments, the one or more mutations in the RB1 gene result in one or more of an insertion, deletion or substitution of one or more amino acid residues in a polypeptide encoded by the RB1 gene. In some embodiments, the one or more mutations in the RB1 gene are detected in the sample by one or more of: an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the one or more mutations in the RB1 gene are detected in a polypeptide encoded by the RB1 gene. In some embodiments, the one or more mutations in the RB1 gene are detected in the sample by one or more of immunoblotting, enzyme linked immunosorbent assay (ELISA), immunohistochemistry, or mass spectrometry. In some embodiments, detecting a TMB of at least about 10 mut/Mb or at least about 20 mut/Mb comprises measuring the level of TMB in the sample.

In some embodiments, TMB is measured in the sample by whole exome sequencing, whole genome sequencing, or gene-targeted sequencing. In some embodiments, TMB is measured on about 0.80 Mb of sequenced DNA. In some embodiments, TMB is measured on between about 0.83 Mb and about 1.14 Mb of sequenced DNA. In some embodiments, TMB is measured on about 1.1 Mb of sequenced DNA. In some embodiments, TMB is measured on up to about 1.1 Mb of sequenced DNA. In some embodiments, detecting a PD-L1-positive UCB comprises measuring the level of PD-L1 expression in the sample. In some embodiments, the level of PD-L1 expression is measured using an immunohistochemistry assay. In some embodiments, the level of PD-L1 expression is determined based on PD-L1 expression on tumor cells. In some embodiments, the PD-L1-positive UCB comprises about 50% or more PD-L1-positive tumor cells, wherein the percent of PD-L1-positive tumor cells is determined in the sample. In some embodiments, the immunotherapy or the RB1-targeted therapy is a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the RB1-targeted therapy comprises one or more of a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor. In some embodiments, the immunotherapy comprises one or more of a checkpoint inhibitor, a cancer vaccine, a cell-based therapy, a T cell receptor (TCR)-based therapy, an adjuvant immunotherapy, a cytokine immunotherapy, or an oncolytic virus therapy. In some embodiments, the UCB is chemorefractory UCB. In some embodiments, the UCB is metastatic UCB. In some embodiments, the treatment or the one or more treatment options further comprise an additional anti-cancer treatment. In some embodiments, the additional anti-cancer treatment comprises one or more of a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments, the additional anti-cancer treatment comprises one or more of a surgery, radiotherapy, chemotherapy, anti-angiogenic therapy, anti-DNA repair therapy, anti-inflammatory therapy, an anti-neoplastic agent, a chemotherapeutic agent, a growth inhibitory agent, an anti-angiogenic agent, or a cytotoxic agent. In some embodiments, the sample from the individual comprises fluid, cells, or tissue. In some embodiments, the sample from the individual comprises a tumor biopsy or a circulating tumor cell. In some embodiments, the sample from the individual comprises one or more nucleic acids. In some embodiments, the sample from the individual comprises mRNA, genomic DNA, circulating tumor DNA, cell-free DNA, or cell-free RNA. In some embodiments, the sample from the individual comprises one or more polypeptides. In some embodiments, the sample is a formalin-fixed paraffin-embedded (FFPE) sample. In some embodiments, the sample comprises one or more nucleic acids and/or one or more polypeptides obtained from an FFPE sample from the individual. In some embodiments, the one or more nucleic acids comprise mRNA and/or genomic DNA. In some embodiments, the sample is derived from a UCB in the individual. In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the bait comprises a capture nucleic acid molecule configured to hybridize to the one or more nucleic acids. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and 200 nucleotides. In some embodiments, the bait is conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample.

In one aspect, provided herein is a system comprising: a memory configured to store one or more program instructions; and one or more processors configured to execute the one or more program instructions, wherein the one or more program instructions when executed by the one or more processors are configured to: (a) obtain a plurality of sequence reads of one or more nucleic acids, wherein the one or more nucleic acids are derived from a sample obtained from an individual having a urothelial bladder cancer (UCB); (b) analyze the plurality of sequence reads for the presence of a deletion of an methylthioadenosine phosphorylase (MTAP) gene, or of a portion thereof, and one or more mutations in a fibroblast growth factor receptor 3 (FGFR3) gene or in a phosphatase and tensin homolog (PTEN) gene; and (c) detect, based on the analyzing, a deletion of an MTAP gene, or of a portion thereof, and one or more mutations in an FGFR3 gene or in a PTEN gene in the sample.

In one aspect, provided herein is a system comprising: a memory configured to store one or more program instructions; and one or more processors configured to execute the one or more program instructions, wherein the one or more program instructions when executed by the one or more processors are configured to: (a) obtain a plurality of sequence reads of one or more nucleic acids, wherein the one or more nucleic acids are derived from a sample obtained from an individual having a urothelial bladder cancer (UCB); (b) analyze the plurality of sequence reads for the presence of a tumor mutational burden (TMB) of at least about 10 mut/Mb or at least about 20 mut/Mb, and/or one or more mutations in a retinoblastoma (RB1) gene; and (c) detect, based on the analyzing, a TMB of at least about 10 mut/Mb or at least about 20 mut/Mb, and/or one or more mutations in an RB1 gene in the sample.

In one aspect, provided herein is a non-transitory computer readable storage medium comprising one or more programs executable by one or more computer processors for performing a method, wherein the method comprises: (a) obtaining a plurality of sequence reads of one or more nucleic acids, wherein the one or more nucleic acids are derived from a sample obtained from an individual having a urothelial bladder cancer (UCB); (b) analyzing the plurality of sequence reads for the presence of a deletion of an methylthioadenosine phosphorylase (MTAP) gene, or of a portion thereof, and one or more mutations in a fibroblast growth factor receptor 3 (FGFR3) gene or in a phosphatase and tensin homolog (PTEN) gene; and (c) detecting, based on the analyzing, a deletion of an MTAP gene, or of a portion thereof, and one or more mutations in an FGFR3 gene or in a PTEN gene in the sample.

In one aspect, provided herein is a non-transitory computer readable storage medium comprising one or more programs executable by one or more computer processors for performing a method, wherein the method comprises: (a) obtaining a plurality of sequence reads of one or more nucleic acids, wherein the one or more nucleic acids are derived from a sample obtained from an individual having a urothelial bladder cancer (UCB); (b) analyzing the plurality of sequence reads for the presence of a tumor mutational burden (TMB) of at least about 10 mut/Mb or at least about 20 mut/Mb, and/or one or more mutations in a retinoblastoma (RB1) gene; and (c) detecting, based on the analyzing, a TMB of at least about 10 mut/Mb or at least about 20 mut/Mb, and/or one or more mutations in an RB1 gene in the sample.

In one aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a deletion of a methylthioadenosine phosphorylase (MTAP) gene, or of a portion thereof, and one or more mutations in a fibroblast growth factor receptor 3 (FGFR3) gene or in a phosphatase and tensin homolog (PTEN) gene; and (c) detect, based on the analyzing, a deletion of an MTAP gene, or of a portion thereof, and one or more mutations in an FGFR3 gene or in a PTEN gene in the sample. In some embodiments, the sample is obtained from an individual having a urothelial bladder cancer (UCB). In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations result in one or more of an insertion, deletion or substitution of one or more amino acid residues in a polypeptide encoded by the FGFR3 gene or the PTEN gene. In some embodiments, the plurality of sequence reads is obtained by sequencing, whole exome sequencing, whole genome sequencing, gene-targeted sequencing, or next-generation sequencing.

In one aspect, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a deletion of a methylthioadenosine phosphorylase (MTAP) gene, or of a portion thereof, and one or more mutations in a fibroblast growth factor receptor 3 (FGFR3) gene or in a phosphatase and tensin homolog (PTEN) gene; and (c) detecting, using the one or more processors and based on the analyzing, a deletion of an MTAP gene, or of a portion thereof, and one or more mutations in an FGFR3 gene or in a PTEN gene, in the sample. In some embodiments, the sample is obtained from an individual having a urothelial bladder cancer (UCB). In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations result in one or more of an insertion, deletion or substitution of one or more amino acid residues in a polypeptide encoded by the FGFR3 gene or the PTEN gene. In some embodiments, the plurality of sequence reads is obtained by sequencing, whole exome sequencing, whole genome sequencing, gene-targeted sequencing, or next-generation sequencing.

In one aspect, provided herein is a system comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a tumor mutational burden (TMB) of at least about 10 mut/Mb or at least about 20 mut/Mb, and/or one or more mutations in a retinoblastoma (RB1) gene; and (c) detect, based on the analyzing, a tumor mutational burden (TMB) of at least about 10 mut/Mb or at least about 20 mut/Mb, and/or one or more mutations in a retinoblastoma (RB1) gene. In some embodiments, the sample is obtained from an individual having a urothelial bladder cancer (UCB). In some embodiments, the analyzing for the presence of a tumor mutational burden (TMB) is based on about 0.80 Mb of sequenced DNA. In some embodiments, the analyzing for the presence of a tumor mutational burden (TMB) is based on between about 0.83 Mb and about 1.14 Mb of sequenced DNA. In some embodiments, the analyzing for the presence of a tumor mutational burden (TMB) is based on up to about 1.1 Mb of sequenced DNA. In some embodiments, the one or more mutations in the RB1 gene comprise one or more of an insertion, deletion or substitution of one or more nucleotides, a genomic rearrangement, an alteration in a promoter of the RB1 gene, a gene fusion, or a copy number alteration. In some embodiments, the one or more mutations in the RB1 gene result in one or more of an insertion, deletion or substitution of one or more amino acid residues in a polypeptide encoded by the RB1 gene. In some embodiments, the plurality of sequence reads is obtained by sequencing, whole exome sequencing, whole genome sequencing, gene-targeted sequencing, or next-generation sequencing.

In one aspect, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a tumor mutational burden (TMB) of at least about 10 mut/Mb or at least about 20 mut/Mb, and/or one or more mutations in a retinoblastoma (RB1) gene; and (c) detecting, using the one or more processors and based on the analyzing, a tumor mutational burden (TMB) of at least about 10 mut/Mb or at least about 20 mut/Mb, and/or one or more mutations in a retinoblastoma (RB1) gene, in the sample. In some embodiments, the sample is obtained from an individual having a urothelial bladder cancer (UCB). In some embodiments, the analyzing for the presence of a tumor mutational burden (TMB) is based on about 0.80 Mb of sequenced DNA. In some embodiments, the analyzing for the presence of a tumor mutational burden (TMB) is based on between about 0.83 Mb and about 1.14 Mb of sequenced DNA. In some embodiments, the analyzing for the presence of a tumor mutational burden (TMB) is based on up to about 1.1 Mb of sequenced DNA. In some embodiments, the one or more mutations in the RB1 gene comprise one or more of an insertion, deletion or substitution of one or more nucleotides, a genomic rearrangement, an alteration in a promoter of the RB1 gene, a gene fusion, or a copy number alteration. In some embodiments, the one or more mutations in the RB1 gene result in one or more of an insertion, deletion or substitution of one or more amino acid residues in a polypeptide encoded by the RB1 gene. In some embodiments, the plurality of sequence reads is obtained by sequencing, whole exome sequencing, whole genome sequencing, gene-targeted sequencing, or next-generation sequencing.

In one aspect, provided herein is an anti-cancer therapy for use in a method of treating or delaying progression of a UCB, wherein the method comprises administering an anti-cancer therapy to an individual having a UCB, wherein a deletion of an MTAP gene, or of a portion thereof, is detected in a sample obtained from the individual.

In one aspect, provided herein is an anti-cancer therapy for use in the manufacture of a medicament for treating or delaying progression of a UCB, wherein the medicament is to be administered to an individual having a UCB, wherein a deletion of an MTAP gene, or of a portion thereof, has been detected in a sample obtained from the individual.

In one aspect, provided herein is an FGFR-targeted therapy for use in a method of treating or delaying progression of a UCB, wherein the method comprises administering an FGFR-targeted therapy to an individual having a UCB, wherein a deletion of an MTAP gene, or of a portion thereof, and one or more mutations in an FGFR3 gene are detected in a sample obtained from the individual.

In one aspect, provided herein is a PTEN-targeted therapy for use in a method of treating or delaying progression of a UCB, wherein the method comprises administering a PTEN-targeted therapy to an individual having a UCB, wherein a deletion of an MTAP gene, or of a portion thereof, and one or more mutations in a PTEN gene are detected in a sample obtained from the individual.

In one aspect, provided herein is an immunotherapy for use in a method of treating or delaying progression of a UCB, wherein the method comprises administering an immunotherapy to an individual having a UCB, wherein a TMB of at least about 10 mut/Mb or at least about 20 mut/Mb, and/or a PD-L1-positive UCB are detected in a sample obtained from the individual.

In some embodiments, the absence of a deletion of an MTAP gene, or of a portion thereof, is further detected in the sample.

In one aspect, provided herein is an RB1-targeted therapy for use in a method of treating or delaying progression of a UCB, wherein the method comprises administering an RB1-targeted therapy to an individual having a UCB, wherein one or more mutations in an RB1 gene are detected in a sample obtained from the individual.

In some embodiments, the absence of a deletion of an MTAP gene, or of a portion thereof, is further detected in the sample.

In one aspect, provided herein is an FGFR-targeted therapy for use in the manufacture of a medicament for treating or delaying progression of a UCB, wherein the medicament is to be administered to an individual having a UCB, wherein a deletion of an MTAP gene, or of a portion thereof, and one or more mutations in an FGFR3 gene have been detected in a sample obtained from the individual.

In one aspect, provided herein is a PTEN-targeted therapy for use in the manufacture of a medicament for treating or delaying progression of a UCB, wherein the medicament is to be administered to an individual having a UCB, wherein a deletion of an MTAP gene, or of a portion thereof, and one or more mutations in a PTEN gene have been detected in a sample obtained from the individual.

In one aspect, provided herein is an immunotherapy for use in the manufacture of a medicament for treating or delaying progression of a UCB, wherein the medicament is to be administered to an individual having a UCB, wherein a TMB of at least about 10 mut/Mb or at least about 20 mut/Mb, and/or a PD-L1-positive UCB have been detected in a sample obtained from the individual.

In some embodiments, the absence of a deletion of an MTAP gene, or of a portion thereof, is further detected in the sample.

In one aspect, provided herein is an RB1-targeted therapy for use in the manufacture of a medicament for treating or delaying progression of a UCB, wherein the medicament is to be administered to an individual having a UCB, wherein one or more mutations in an RB1 gene have been detected in a sample obtained from the individual.

In some embodiments, the absence of a deletion of an MTAP gene, or of a portion thereof, is further detected in the sample.

In another aspect, provided herein is a method of identifying an individual having a non-small cell lung cancer (NSCLC) who may benefit from a treatment comprising an epidermal growth factor receptor (EGFR)-targeted therapy or a SMARCA4-targeted therapy, the method comprising detecting in a sample from the individual a deletion of a methylthioadenosine phosphorylase (MTAP) gene, or of a portion thereof, and one or more mutations in an EGFR gene or in a SMARCA4 gene, wherein: (a) the presence of the deletion of the MTAP gene, or of the portion thereof, and one or more mutations in the EGFR gene in the sample identifies the individual as one who may benefit from a treatment comprising an EGFR-targeted therapy; or (b) the presence of the deletion of the MTAP gene, or of the portion thereof, and one or more mutations in the SMARCA4 gene in the sample identifies the individual as one who may benefit from a treatment comprising a SMARCA4-targeted therapy.

In another aspect, provided herein is a method of selecting a treatment for an individual having a NSCLC, the method comprising detecting a deletion of an MTAP gene, or of a portion thereof, and one or more mutations in an EGFR gene or in a SMARCA4 gene in a sample from the individual, wherein: (a) the presence of the deletion of the MTAP gene, or of the portion thereof, and one or more mutations in the EGFR gene in the sample identifies the individual as one who may benefit from a treatment comprising an EGFR-targeted therapy; or (b) the presence of the deletion of the MTAP gene, or of the portion thereof, and one or more mutations in the SMARCA4 gene in the sample identifies the individual as one who may benefit from a treatment comprising a SMARCA4-targeted therapy.

In some embodiments, the presence in the sample of the deletion of the MTAP gene, or of the portion thereof, and the one or more mutations in the EGFR gene or in the SMARCA4 gene identifies the individual as one who may benefit from an additional treatment comprising a protein arginine methyltransferase 5 (PRMT5)-targeted therapy.

In another aspect, provided herein is a method of identifying one or more treatment options for an individual having a NSCLC, the method comprising: (a) detecting a deletion of an MTAP gene, or of a portion thereof, and one or more mutations in an EGFR gene or in a SMARCA4 gene in a sample from the individual; and (b) generating a report, wherein the report comprises: (i) one or more treatment options identified for the individual based at least in part on the presence of the deletion of the MTAP gene, or of the portion thereof, and one or more mutations in the EGFR gene in the sample, wherein the one or more treatment options comprise an EGFR-targeted therapy; or (ii) one or more treatment options identified for the individual based at least in part on the presence of the deletion of the MTAP gene, or of the portion thereof, and one or more mutations in the SMARCA4 gene in the sample, wherein the one or more treatment options comprise a SMARCA4-targeted therapy.

In another aspect, provided herein is a method of identifying one or more treatment options for an individual having a NSCLC, the method comprising acquiring knowledge of a deletion of an MTAP gene, or of a portion thereof, and one or more mutations in an EGFR gene or in a SMARCA4 gene in a sample from the individual; and generating a report comprising: (a) one or more treatment options identified for the individual based at least in part on the knowledge of a deletion of the MTAP gene, or of a portion thereof, and one or more mutations in the EGFR gene in the sample, wherein the one or more treatment options comprise an EGFR-targeted therapy; or (b) one or more treatment options identified for the individual based at least in part on the knowledge of a deletion of the MTAP gene, or of a portion thereof, and one or more mutations in the SMARCA4 gene in the sample, wherein the one or more treatment options comprise a SMARCA4-targeted therapy.

In some embodiments, the one or more treatment options identified for the individual further comprise a PRMT5-targeted therapy.

In another aspect, provided herein is a method of selecting a treatment for an individual having a NSCLC, comprising acquiring knowledge of a deletion of an MTAP gene, or of a portion thereof, and one or more mutations in an EGFR gene or in a SMARCA4 gene in a sample from the individual, wherein: (a) responsive to the acquisition of knowledge of a deletion of an MTAP gene, or of a portion thereof, and one or more mutations in an EGFR gene in the sample: (i) the individual is classified as a candidate to receive a treatment comprising an EGFR-targeted therapy; and/or (ii) the individual is identified as likely to respond to a treatment that comprises an EGFR-targeted therapy; or (b) responsive to the acquisition of knowledge of a deletion of an MTAP gene, or of a portion thereof, and one or more mutations in a SMARCA4 gene in the sample: (i) the individual is classified as a candidate to receive a treatment comprising a SMARCA4-targeted therapy; and/or (ii) the individual is identified as likely to respond to a treatment that comprises a SMARCA4-targeted therapy.

In some embodiments, the treatment further comprises a PRMT5-targeted therapy.

In another aspect, provided herein is a method of predicting survival of an individual having a NSCLC treated with a treatment comprising an EGFR-targeted therapy or a SMARCA4-targeted therapy, the method comprising acquiring knowledge of a deletion of an MTAP gene, or of a portion thereof, and one or more mutations in an EGFR gene or in a SMARCA4 gene in a sample from the individual, wherein responsive to the acquisition of said knowledge, the individual is predicted to have longer survival after treatment with the EGFR-targeted therapy or the SMARCA4-targeted therapy, as compared to an individual whose NSCLC does not exhibit a deletion of an MTAP gene, or of a portion thereof, and one or more mutations in an EGFR gene or in a SMARCA4 gene.

In another aspect, provided herein is a method of treating or delaying progression of a NSCLC, comprising: (a) acquiring knowledge of a deletion of an MTAP gene, or of a portion thereof, and one or more mutations in an EGFR gene in a sample from an individual, and responsive to said knowledge, administering to the individual an effective amount of a treatment comprising an EGFR-targeted therapy; or (b) acquiring knowledge of a deletion of an MTAP gene, or of a portion thereof, and one or more mutations in a SMARCA4 gene in a sample from an individual, and responsive to said knowledge, administering to the individual an effective amount of a treatment comprising a SMARCA4-targeted therapy.

In another aspect, provided herein is a method of treating or delaying progression of a NSCLC, comprising: (a) responsive to acquiring knowledge of a deletion of an MTAP gene, or of a portion thereof, and one or more mutations in an EGFR gene in a sample from an individual, administering to the individual an effective amount of a treatment comprising an EGFR-targeted therapy; or (b) responsive to acquiring knowledge of a deletion of an MTAP gene, or of a portion thereof, and one or more mutations in a SMARCA4 gene in a sample from an individual, administering to the individual an effective amount of a treatment comprising a SMARCA4-targeted therapy.

In some embodiments, the acquiring knowledge of the deletion of the MTAP gene, or of the portion thereof, and one or more mutations in the EGFR gene or in the SMARCA4 gene comprises detecting the deletion of the MTAP gene, or of the portion thereof, and the one or more mutations in the EGFR gene or in the SMARCA4 gene in the sample.

In another aspect, provided herein is a method of treating or delaying progression of a NSCLC, comprising: (a) detecting a deletion of an MTAP gene, or of a portion thereof, and one or more mutations in an EGFR gene in a sample from an individual, and administering to the individual an effective amount of a treatment comprising an EGFR-targeted therapy; or (b) detecting a deletion of an MTAP gene, or of a portion thereof, and one or more mutations in a SMARCA4 gene in a sample from an individual, and administering to the individual an effective amount of a treatment comprising a SMARCA4-targeted therapy.

In another aspect, provided herein is a method of detecting the presence or absence of a NSCLC in an individual, comprising: (a) detecting the presence or absence of a NSCLC in a sample from an individual; and (i) detecting the presence or absence of a deletion of an MTAP gene, or of a portion thereof, and one or more mutations in an EGFR gene in the sample, or (ii) detecting the presence or absence of a deletion of an MTAP gene, or of a portion thereof, and one or more mutations in a SMARCA4 gene in the sample.

In another aspect, provided herein is a method of assessing a NSCLC in an individual, the method comprising: (a) detecting a deletion of an MTAP gene, or of a portion thereof, and one or more mutations in an EGFR gene or in a SMARCA4 gene in a sample from the individual; and (b) providing an assessment of the deletion of the MTAP gene, or of the portion thereof, and the one or more mutations in the EGFR gene or in the SMARCA4 gene.

In another aspect, provided herein is a method of detecting a deletion of an MTAP gene, or of a portion thereof, and one or more mutations in an EGFR gene or in a SMARCA4 gene, the method comprising detecting the deletion of the MTAP gene, or of the portion thereof, and the one or more mutations in the EGFR gene or in the SMARCA4 gene in a sample from an individual having a NSCLC.

In another aspect, provided herein is a method of detecting a deletion of an MTAP gene, or of a portion thereof, and one or more mutations in an EGFR gene or in a SMARCA4 gene, the method comprising: (a) providing a sample from an individual having a NSCLC, wherein the sample comprises one or more nucleic acids; (b) preparing a nucleic acid sequencing library from the one or more nucleic acids in the sample; (c) amplifying said library using a polymerase chain reaction (PCR); (d) selectively enriching for one or more nucleic acids comprising MTAP, EGFR, or SMARCA4 gene nucleotide sequences in said library to produce an enriched sample; (e) sequencing the enriched sample, thereby producing a plurality of sequencing reads; (f) analyzing the plurality of sequencing reads for the presence of a deletion of an MTAP gene, or of a portion thereof, and one or more mutations in an EGFR gene or in a SMARCA4 gene; (g) detecting, based on the analyzing step, a deletion of an MTAP gene, or of a portion thereof, and one or more mutations in an EGFR gene or in a SMARCA4 gene in the sample from the individual.

In some embodiments, the methods provided herein further comprise selectively enriching for one or more of: (a) one or more nucleic acids comprising nucleotide sequences that comprise the deletion of the MTAP gene, or of the portion thereof, (b) one or more nucleic acids comprising nucleotide sequences that comprise the one or more mutations in the EGFR gene, or (c) one or more nucleic acids comprising nucleotide sequences that comprise the one or more mutations in the SMARCA4 gene; wherein the selectively enriching produces an enriched sample.

In another aspect, provided herein is a method of treating or delaying progression of a NSCLC, comprising: (a) administering to an individual having a NSCLC an effective amount of a treatment comprising an EGFR-targeted therapy, wherein the NSCLC comprises a deletion of an MTAP gene, or of a portion thereof, and one or more mutations in an EGFR gene; or (b) administering to an individual having a NSCLC an effective amount of a treatment comprising a SMARCA4-targeted therapy, wherein the NSCLC comprises a deletion of an MTAP gene, or of a portion thereof, and one or more mutations in a SMARCA4 gene.

In some embodiments, the methods further comprise acquiring knowledge of or detecting in the sample a deletion of a CDKN2A gene, or of a portion thereof; and/or a deletion of a CDKN2B gene, or of a portion thereof.

In some embodiments, the methods comprise acquiring knowledge of or detecting in the sample one or more mutations in one or more genes, wherein the one or more genes comprise TP53, KRAS, ALK, ROS1, NTRK1, STK11, KEAP1, PIK3CA, PTEN, MET, ERBB2, BRAF, or RB1. In some embodiments, the individual has one or more mutations in one or more genes, wherein the one or more genes comprise TP53, KRAS, ALK, ROS1, NTRK1, STK11, KEAP1, PIK3CA, PTEN, MET, ERBB2, BRAF, or RB1. In some embodiments, the methods comprise administering to the individual an effective amount of a treatment comprising an anti-cancer therapy. In some embodiments, the methods further comprise selectively enriching for one or more nucleic acids comprising nucleotide sequences that comprise one or more mutations in one or more genes, wherein the one or more genes comprise TP53, KRAS, ALK, ROS1, NTRK1, STK11, KEAP1, PIK3CA, PTEN, MET, ERBB2, BRAF, or RB1, to produce an enriched sample. In some embodiments, the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

In some embodiments, the individual has a deletion of a CDKN2A gene, or of a portion thereof; and/or a deletion of a CDKN2B gene, or of a portion thereof. In some embodiments, the individual has a heterozygous or a homozygous deletion of a CDKN2A gene, or of a portion thereof; and/or a heterozygous or a homozygous deletion of a CDKN2B gene, or of a portion thereof.

In some embodiments, the individual has a heterozygous or a homozygous deletion of the MTAP gene, or of a portion thereof.

In some embodiments, the methods further comprise administering to the individual a PRMT5-targeted therapy. In some embodiments, the individual is being treated with a PRMT5-targeted therapy. In some embodiments, the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

In some embodiments, the one or more mutations in the EGFR gene or the SMARCA4 gene comprise one or more of an insertion, deletion or substitution of one or more nucleotides, a genomic rearrangement, an alteration in a promoter, a gene fusion, or a copy number alteration. In some embodiments, the one or more mutations in the EGFR gene or the SMARCA4 gene result in one or more of an insertion, deletion or substitution of one or more amino acid residues in a polypeptide encoded by the EGFR gene or the SMARCA4 gene.

In some embodiments, the EGFR-targeted therapy or the SMARCA4-targeted therapy is a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the EGFR-targeted therapy comprises one or more of an EGFR inhibitor, an HSP90 inhibitor, a VEGFR/EGFR dual inhibitor, a MEK inhibitor, or a Raf inhibitor. In some embodiments, the SMARCA4-targeted therapy comprises one or more of a CDK4/6 inhibitor, an Aurora kinase (AURK) inhibitor, an ATR inhibitor, an EZH2 inhibitor, a KDM6 inhibitor, a kinase inhibitor, a cisplatin-based chemotherapy, or an immune checkpoint inhibitor.

In some embodiments, the deletion of the MTAP gene or of the portion thereof, or the one or more mutations in the EGFR gene or the SMARCA4 gene are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the deletion of the MTAP gene or of the portion thereof, or the one or more mutations in the EGFR gene or the SMARCA4 gene are detected in a polypeptide encoded by the MTAP gene, the EGFR gene, or the SMARCA4 gene. In some embodiments, the deletion of the MTAP gene or of the portion thereof, or the one or more mutations in the EGFR gene or the SMARCA4 gene are detected in the sample by one or more of: immunoblotting, enzyme linked immunosorbent assay (ELISA), immunohistochemistry, or mass spectrometry. In some embodiments, the deletion of the CDKN2A gene or of the portion thereof, and/or the deletion of the CDKN2B gene or of the portion thereof, are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the deletion of the CDKN2A gene or of the portion thereof, and/or the deletion of the CDKN2B gene or of the portion thereof, are detected in a polypeptide encoded by the CDKN2A gene or the CDKN2B gene. In some embodiments, the deletion of the CDKN2A gene or of the portion thereof, and/or the deletion of the CDKN2B gene or of the portion thereof, are detected in the sample by one or more of: immunoblotting, enzyme linked immunosorbent assay (ELISA), immunohistochemistry, or mass spectrometry.

In another aspect, provided herein is a method of identifying an individual having a non-small cell lung cancer (NSCLC) who may benefit from a treatment comprising an immunotherapy, a retinoblastoma (RB1)-targeted therapy, a KRAS-targeted therapy, or a TP53-targeted therapy, the method comprising detecting in a sample from the individual one or more of: (a) a tumor mutational burden (TMB) of at least about 10 mut/Mb or at least about 20 mut/Mb, (b) a PD-L1-positive NSCLC, or (c) one or more mutations in an RB1 gene, a KRAS gene, or a TP53 gene; wherein: (i) the presence in the sample of a TMB of at least about 10 mut/Mb or at least about 20 mut/Mb, or of a PD-L1-positive NSCLC identifies the individual as one who may benefit from a treatment comprising an immunotherapy; (ii) the presence in the sample of one or more mutations in the RB1 gene identifies the individual as one who may benefit from a treatment comprising an RB1-targeted therapy; (iii) the presence in the sample of one or more mutations in the KRAS gene identifies the individual as one who may benefit from a treatment comprising a KRAS-targeted therapy; or (iv) the presence in the sample of one or more mutations in the TP53 gene identifies the individual as one who may benefit from a treatment comprising a TP53-targeted therapy.

In another aspect, provided herein is a method of selecting a treatment for an individual having a NSCLC, the method comprising detecting one or more of: (a) a TMB of at least about 10 mut/Mb or at least about 20 mut/Mb, (b) a PD-L1-positive NSCLC, or (c) one or more mutations in an RB1 gene, a KRAS gene, or a TP53 gene, in a sample from the individual; wherein: (i) the presence in the sample of a TMB of at least about 10 mut/Mb or at least about 20 mut/Mb, or of a PD-L1-positive NSCLC identifies the individual as one who may benefit from a treatment comprising an immunotherapy; (ii) the presence in the sample of one or more mutations in the RB1 gene identifies the individual as one who may benefit from a treatment comprising an RB1-targeted therapy; (iii) the presence in the sample of one or more mutations in the KRAS gene identifies the individual as one who may benefit from a treatment comprising a KRAS-targeted therapy; or (iv) the presence in the sample of one or more mutations in the TP53 gene identifies the individual as one who may benefit from a treatment comprising a TP53-targeted therapy.

In another aspect, provided herein is a method of identifying one or more treatment options for an individual having a NSCLC, the method comprising: (a) detecting in a sample from the individual one or more of: (i) a TMB of at least about 10 mut/Mb or at least about 20 mut/Mb, (ii) a PD-L1-positive NSCLC, or (iii) one or more mutations in an RB1 gene, a KRAS gene, or a TP53 gene; and (b) generating a report, wherein the report comprises: (i) one or more treatment options identified for the individual based at least in part on the presence of a TMB of at least about 10 mut/Mb or at least about 20 mut/Mb, or of a PD-L1-positive NSCLC in the sample, wherein the one or more treatment options comprise an immunotherapy; (ii) one or more treatment options identified for the individual based at least in part on the presence of one or more mutations in the RB1 gene in the sample, wherein the one or more treatment options comprise an RB1-targeted therapy; (iii) one or more treatment options identified for the individual based at least in part on the presence of one or more mutations in the KRAS gene in the sample, wherein the one or more treatment options comprise a KRAS-targeted therapy; or (iv) one or more treatment options identified for the individual based at least in part on the presence of one or more mutations in the TP53 gene in the sample, wherein the one or more treatment options comprise a TP53-targeted therapy.

In another aspect, provided herein is a method of identifying one or more treatment options for an individual having a NSCLC, the method comprising acquiring knowledge of one or more of: (a) a TMB of at least about 10 mut/Mb or at least about 20 mut/Mb, (b) a PD-L1-positive NSCLC, or (c) one or more mutations in an RB1 gene, a KRAS gene, or a TP53 gene, in a sample from the individual; and generating a report comprising: (i) one or more treatment options identified for the individual based at least in part on the knowledge of a TMB of at least about 10 mut/Mb or at least about 20 mut/Mb, or of a PD-L1-positive NSCLC in the sample, wherein the one or more treatment options comprise an immunotherapy; (ii) one or more treatment options identified for the individual based at least in part on the knowledge of one or more mutations in the RB1 gene in the sample, wherein the one or more treatment options comprise an RB1-targeted therapy; (iii) one or more treatment options identified for the individual based at least in part on the knowledge of one or more mutations in the KRAS gene in the sample, wherein the one or more treatment options comprise a KRAS-targeted therapy; or (iv) one or more treatment options identified for the individual based at least in part on the knowledge of one or more mutations in the TP53 gene in the sample, wherein the one or more treatment options comprise a TP53-targeted therapy.

In another aspect, provided herein is a method of selecting a treatment for an individual having a NSCLC, comprising acquiring knowledge of one or more of: (a) a TMB of at least about 10 mut/Mb or at least about 20 mut/Mb, (b) a PD-L1-positive NSCLC, or (c) one or more mutations in an RB1 gene, a KRAS gene, or a TP53 gene, in a sample from the individual, wherein: (a) responsive to acquisition of knowledge of a TMB of at least about 10 mut/Mb or at least about 20 mut/Mb, or of a PD-L1-positive NSCLC in the sample: (i) the individual is classified as a candidate to receive a treatment comprising an immunotherapy; and/or (ii) the individual is identified as likely to respond to a treatment that comprises an immunotherapy; (b) responsive to acquisition of knowledge of one or more mutations in the RB1 gene in the sample: (i) the individual is classified as a candidate to receive a treatment comprising an RB1-targeted therapy; and/or (ii) the individual is identified as likely to respond to a treatment that comprises an RB1-targeted therapy; (c) responsive to acquisition of knowledge of one or more mutations in the KRAS gene in the sample: (i) the individual is classified as a candidate to receive a treatment comprising a KRAS-targeted therapy; and/or (ii) the individual is identified as likely to respond to a treatment that comprises a KRAS-targeted therapy; or (d) responsive to acquisition of knowledge of one or more mutations in the TP53 gene in the sample: (i) the individual is classified as a candidate to receive a treatment comprising a TP53-targeted therapy; and/or (ii) the individual is identified as likely to respond to a treatment that comprises a TP53-targeted therapy.

In another aspect, provided herein is a method of predicting survival of an individual having NSCLC treated with a treatment comprising an immunotherapy, an RB1-targeted therapy, a KRAS-targeted therapy, or a TP53-targeted therapy, the method comprising acquiring knowledge of one or more of: (a) a TMB of at least about 10 mut/Mb or at least about 20 mut/Mb, (b) a PD-L1-positive NSCLC, or (c) one or more mutations in an RB1 gene, a KRAS gene, or a TP53 gene, in a sample from the individual; wherein responsive to acquisition of said knowledge, the individual is predicted to have longer survival after treatment with the immunotherapy, RB1-targeted therapy, KRAS-targeted therapy, or TP53-targeted therapy, as compared to an individual whose NSCLC does not exhibit a TMB of at least about 10 mut/Mb or at least about 20 mut/Mb, a PD-L1-positive NSCLC, or one or more mutations in the RB1 gene, KRAS gene, or TP53 gene.

In another aspect, provided herein is a method of treating or delaying progression of a NSCLC, comprising: (a) acquiring knowledge of a TMB of at least about 10 mut/Mb or at least about 20 mut/Mb, or a PD-L1-positive NSCLC in a sample from an individual, and responsive to said knowledge, administering to the individual an effective amount of a treatment comprising an immunotherapy; (b) acquiring knowledge of one or more mutations in an RB1 gene in a sample from an individual, and responsive to said knowledge, administering to the individual an effective amount of a treatment comprising an RB1-targeted therapy; (c) acquiring knowledge of one or more mutations in a KRAS gene in a sample from an individual, and responsive to said knowledge, administering to the individual an effective amount of a treatment comprising a KRAS-targeted therapy; or (d) acquiring knowledge of one or more mutations in a TP53 gene in a sample from an individual, and responsive to said knowledge, administering to the individual an effective amount of a treatment comprising a TP53-targeted therapy.

In another aspect, provided herein is a method of treating or delaying progression of a NSCLC, comprising: (a) responsive to acquiring knowledge of a TMB of at least about 10 mut/Mb or at least about 20 mut/Mb, or a PD-L1-positive NSCLC in a sample from an individual, administering to the individual an effective amount of a treatment comprising an immunotherapy; (b) responsive to acquiring knowledge of one or more mutations in an RB1 gene in a sample from an individual, administering to the individual an effective amount of a treatment comprising an RB1-targeted therapy; (c) responsive to acquiring knowledge of one or more mutations in a KRAS gene in a sample from an individual, administering to the individual an effective amount of a treatment comprising a KRAS-targeted therapy; or (d) responsive to acquiring knowledge of one or more mutations in a TP53 gene in a sample from an individual, administering to the individual an effective amount of a treatment comprising a TP53-targeted therapy.

In some embodiments, acquiring knowledge of a TMB of at least about 10 mut/Mb or at least about 20 mut/Mb, a PD-L1-positive NSCLC, or one or more mutations in an RB1 gene, a KRAS gene, or a TP53 gene comprises detecting the TMB of at least about 10 mut/Mb or at least about 20 mut/Mb, the PD-L1-positive NSCLC, or the one or more mutations in the RB1 gene, KRAS gene, or TP53 gene in the sample.

In another aspect, provided herein is a method of treating or delaying progression of a NSCLC, comprising: (a) detecting a TMB of at least about 10 mut/Mb or at least about 20 mut/Mb, or a PD-L1-positive NSCLC in a sample from an individual, and administering to the individual an effective amount of a treatment comprising an immunotherapy; (b) detecting one or more mutations in an RB1 gene in a sample from an individual, and administering to the individual an effective amount of a treatment comprising an RB1-targeted therapy; (c) detecting one or more mutations in a KRAS gene in a sample from an individual, and administering to the individual an effective amount of a treatment comprising a KRAS-targeted therapy; or (d) detecting one or more mutations in a TP53 gene in a sample from an individual, and administering to the individual an effective amount of a treatment comprising a TP53-targeted therapy.

In another aspect, provided herein is a method of detecting the presence or absence of a NSCLC in an individual, comprising: (a) detecting the presence or absence of a NSCLC in a sample from an individual; and (i) detecting the presence or absence of a TMB of at least about 10 mut/Mb or at least about 20 mut/Mb, or a PD-L1-positive NSCLC in the sample, (ii) detecting the presence or absence of one or more mutations in an RB1 gene in the sample, (iii) detecting the presence or absence of one or more mutations in a KRAS gene in the sample, or (iv) detecting the presence or absence of one or more mutations in a TP53 gene in the sample.

In another aspect, provided herein is a method of assessing a NSCLC in an individual, the method comprising: (a) detecting one or more of: (i) a TMB of at least about 10 mut/Mb or at least about 20 mut/Mb, (ii) a PD-L1-positive NSCLC, or (iii) one or more mutations in an RB1 gene, a KRAS gene, or a TP53 gene, in a sample from the individual; and (b) providing an assessment of the TMB of at least about 10 mut/Mb or at least about 20 mut/Mb, the PD-L1-positive NSCLC, or the one or more mutations in the RB1 gene, KRAS gene, or TP53 gene in the sample.

In another aspect, provided herein is a method of detecting a TMB of at least about 10 mut/Mb or at least about 20 mut/Mb, a PD-L1-positive NSCLC, or one or more mutations in an RB1 gene, a KRAS gene, or a TP53 gene, the method comprising detecting the TMB of at least about 10 mut/Mb or at least about 20 mut/Mb, the PD-L1-positive NSCLC, or the one or more mutations in the RB1 gene, KRAS gene, or TP53 gene in a sample from an individual having a NSCLC.

In another aspect, provided herein is a method of detecting one or more mutations in an RB1 gene, a KRAS gene, or a TP53 gene, the method comprising: (a) providing a sample from an individual having a NSCLC, wherein the sample comprises one or more nucleic acids; (b) preparing a nucleic acid sequencing library from the one or more nucleic acids in the sample; (c) amplifying said library using a polymerase chain reaction (PCR); (d) selectively enriching for one or more nucleic acids comprising RB1 gene, KRAS gene, or TP53 gene nucleotide sequences in said library to produce an enriched sample; (e) sequencing the enriched sample, thereby producing a plurality of sequencing reads; (f) analyzing the plurality of sequencing reads for the presence of one or more mutations in an RB1 gene, a KRAS gene, or a TP53 gene; (g) detecting, based on the analyzing step, one or more mutations in an RB1 gene, a KRAS gene, or a TP53 gene in the sample from the individual.

In another aspect, provided herein is a method of detecting a TMB of at least about 10 mut/Mb or at least about 20 mut/Mb, the method comprising: (a) providing a sample from an individual having a NSCLC, wherein the sample comprises one or more nucleic acids; (b) preparing a nucleic acid sequencing library from the one or more nucleic acids in the sample; (c) amplifying said library using a polymerase chain reaction (PCR); (d) sequencing said library, thereby producing a plurality of sequencing reads; (e) analyzing the plurality of sequencing reads for the presence of a TMB of at least about 10 mut/Mb or at least about 20 mut/Mb; (f) detecting, based on the analyzing step, a TMB of at least about 10 mut/Mb or at least about 20 mut/Mb in the sample from the individual.

In another aspect, provided herein is a method of treating or delaying progression of a NSCLC, comprising: (a) administering to an individual having a NSCLC an effective amount of a treatment comprising an immunotherapy, wherein the NSCLC comprises a TMB of at least about 10 mut/Mb or at least about 20 mut/Mb, or wherein the NSCLC is PD-L1-positive; (b) administering to an individual having a NSCLC an effective amount of a treatment comprising an RB1-targeted therapy, wherein the NSCLC comprises one or more mutations in an RB1 gene; (c) administering to an individual having a NSCLC an effective amount of a treatment comprising a KRAS-targeted therapy, wherein the NSCLC comprises one or more mutations in a KRAS gene; or (d) administering to an individual having a NSCLC an effective amount of a treatment comprising a TP53-targeted therapy, wherein the NSCLC comprises one or more mutations in a TP53 gene.

In some embodiments, the methods further comprise acquiring knowledge of the absence of a deletion of an MTAP gene, or of a portion thereof, in the sample. In some embodiments, acquiring knowledge of the absence of a deletion of an MTAP gene, or of a portion thereof, comprises detecting the absence of a deletion of an MTAP gene, or of a portion thereof, in the sample. In some embodiments, the methods further comprise detecting the absence of a deletion of an MTAP gene, or of a portion thereof, in the sample.

In some embodiments, the methods further comprise acquiring knowledge of or detecting in the sample one or more mutations in one or more genes, wherein the one or more genes comprise EGFR, ALK, ROS1, NTRK1, STK11, KEAP1, PIK3CA, SMARCA4, PTEN, MET, ERBB2, or BRAF. In some embodiments, the individual has one or more mutations in one or more genes, wherein the one or more genes comprise EGFR, ALK, ROS1, NTRK1, STK11, KEAP1, PIK3CA, SMARCA4, PTEN, MET, ERBB2, or BRAF. In some embodiments, the methods further comprise administering to the individual an effective amount of a treatment comprising anti-cancer therapy. In some embodiments, the methods comprise selectively enriching for one or more nucleic acids comprising nucleotide sequences that comprise one or more mutations in one or more genes to produce an enriched sample, wherein the one or more genes comprise EGFR, ALK, ROS1, NTRK1, STK11, KEAP1, PIK3CA, SMARCA4, PTEN, MET, ERBB2, or BRAF. In some embodiments, the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the one or more mutations in the one or more genes are detected in a polypeptide encoded by the one or more genes. In some embodiments, the one or more mutations in the one or more genes are detected in the sample by one or more of immunoblotting, enzyme linked immunosorbent assay (ELISA), immunohistochemistry, or mass spectrometry.

In some embodiments, the methods further comprise selectively enriching for one or more nucleic acids comprising MTAP nucleotide sequences to produce an enriched sample. In some embodiments, the absence of a deletion of an MTAP gene, or of a portion thereof, is detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the individual does not have a deletion of an MTAP gene, or of a portion thereof.

In some embodiments, the methods further comprise selectively enriching for one or more nucleic acids comprising nucleotide sequences that comprise the one or more mutations in the RB1 gene, KRAS gene, or TP53 gene to produce an enriched sample. In some embodiments, the one or more mutations in the RB1 gene, KRAS gene, or TP53 gene comprise one or more of an insertion, deletion or substitution of one or more nucleotides, a genomic rearrangement, an alteration in a promoter of the RB1 gene, KRAS gene, or TP53 gene, a gene fusion, or a copy number alteration. In some embodiments, the one or more mutations in the RB1 gene, KRAS gene, or TP53 gene result in one or more of an insertion, deletion or substitution of one or more amino acid residues in a polypeptide encoded by the RB1 gene, KRAS gene, or TP53 gene. In some embodiments, the one or more mutations in the KRAS gene result in a G12C amino acid substitution in a KRAS polypeptide encoded by the KRAS gene. In some embodiments, the one or more mutations in the RB1 gene, KRAS gene, or TP53 gene are detected in the sample by one or more of: an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the one or more mutations in the RB1 gene, KRAS gene, or TP53 gene are detected in a polypeptide encoded by the RB1 gene, KRAS gene, or TP53 gene. In some embodiments, the one or more mutations in the RB1 gene, KRAS gene, or TP53 gene are detected in the sample by one or more of immunoblotting, enzyme linked immunosorbent assay (ELISA), immunohistochemistry, or mass spectrometry.

In some embodiments, detecting a TMB of at least about 10 mut/Mb or at least about 20 mut/Mb comprises measuring the level of TMB in the sample. In some embodiments, TMB is measured in the sample by whole exome sequencing, whole genome sequencing, or gene-targeted sequencing. In some embodiments, TMB is measured on about 0.80 Mb of sequenced DNA. In some embodiments, TMB is measured on between about 0.83 Mb and about 1.14 Mb of sequenced DNA. In some embodiments, TMB is measured on about 1.1 Mb of sequenced DNA. In some embodiments, TMB is measured on up to about 1.1 Mb of sequenced DNA.

In some embodiments, detecting a PD-L1-positive NSCLC comprises measuring the level of PD-L1 expression in the sample. In some embodiments, the level of PD-L1 expression is measured using an immunohistochemistry assay. In some embodiments, the level of PD-L1 expression is determined based on PD-L1 expression on tumor cells. In some embodiments, the PD-L1-positive NSCLC comprises a tumor proportion score (TPS) of between about 1% and about 49%. In some embodiments, the PD-L1-positive NSCLC comprises a tumor proportion score (TPS) of about 50% or greater.

In some embodiments, the immunotherapy, the RB1-targeted therapy, the KRAS-targeted therapy, or the TP53-targeted therapy is a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the RB1-targeted therapy comprises one or more of a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor; the KRAS-targeted therapy comprises one or more of a kinase inhibitor, a farnesyltransferase inhibitor, a geranylgeranyltransferase inhibitor, a palmitoylation inhibitor, an inhibitor of methylation cleavage, or an agent that inhibits the modification or post-translational processing of KRAS; or the TP53-targeted therapy comprises one or more of a p53 reactivator, an mTOR inhibitor, a Pin1 inhibitor, an ATR inhibitor, a proteasome inhibitor, a CHK inhibitor, an ATM inhibitor, a WEE1 inhibitor, or a murine double minute 2 (MDM2) inhibitor. In some embodiments, the immunotherapy comprises one or more of a checkpoint inhibitor, a cancer vaccine, a cell-based therapy, a T cell receptor (TCR)-based therapy, an adjuvant immunotherapy, a cytokine immunotherapy, or an oncolytic virus therapy. In some embodiments, the KRAS-targeted therapy comprises a KRAS (G12C)-targeted therapy. In some embodiments, the KRAS (G12C)-targeted therapy comprises one or more of a KRAS inhibitor, a KRAS (G12C) inhibitor, and/or a SHP2 inhibitor.

In some embodiments, the NSCLC is advanced NSCLC and/or metastatic NSCLC. In some embodiments, the NSCLC has an adenocarcinoma, squamous cell carcinoma, not otherwise specified, large cell neuroendocrine, sarcomatoid, or adenosquamous carcinoma subtype. In some embodiments, the NSCLC comprises a TMB of about 9.4 mut/Mb. In some embodiments, the treatment or the one or more treatment options further comprise an additional anti-cancer treatment. In some embodiments, the additional anti-cancer treatment comprises one or more of a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the additional anti-cancer treatment comprises one or more of a surgery, radiotherapy, chemotherapy, anti-angiogenic therapy, anti-DNA repair therapy, anti-inflammatory therapy, an anti-neoplastic agent, a chemotherapeutic agent, a growth inhibitory agent, an anti-angiogenic agent, or a cytotoxic agent.

In some embodiments, the sample from the individual comprises fluid, cells, or tissue. In some embodiments, the sample from the individual comprises a tumor biopsy or a circulating tumor cell. In some embodiments, the sample from the individual comprises one or more nucleic acids. In some embodiments, the sample from the individual comprises mRNA, genomic DNA, circulating tumor DNA, cell-free DNA, or cell-free RNA. In some embodiments, the sample from the individual comprises one or more polypeptides. In some embodiments, the sample is a formalin-fixed paraffin-embedded (FFPE) sample. In some embodiments, the sample comprises one or more nucleic acids and/or one or more polypeptides obtained from an FFPE sample from the individual. In some embodiments, the one or more nucleic acids comprise mRNA and/or genomic DNA. In some embodiments, the sample is derived from a NSCLC in the individual.

In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the bait comprises a capture nucleic acid molecule configured to hybridize to the one or more nucleic acids. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and 200 nucleotides. In some embodiments, the bait is conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample.

In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a deletion of a methylthioadenosine phosphorylase (MTAP) gene, or of a portion thereof, and one or more mutations in an EGFR gene or in a SMARCA4 gene; and (c) detect, based on the analyzing, a deletion of an MTAP gene, or of a portion thereof, and one or more mutations in an EGFR gene or in a SMARCA4 gene in the sample. In some embodiments, the sample is obtained from an individual having a non-small cell lung cancer (NSCLC). In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations result in one or more of an insertion, deletion or substitution of one or more amino acid residues in a polypeptide encoded by the EGFR gene or the SMARCA4 gene. In some embodiments, the plurality of sequence reads is obtained by sequencing, whole exome sequencing, whole genome sequencing, gene-targeted sequencing, or next-generation sequencing.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a deletion of a methylthioadenosine phosphorylase (MTAP) gene, or of a portion thereof, and one or more mutations in an EGFR gene or in a SMARCA4 gene; and (c) detecting, using the one or more processors and based on the analyzing, a deletion of an MTAP gene, or of a portion thereof, and one or more mutations in an EGFR gene or in a SMARCA4 gene, in the sample. In some embodiments, the sample is obtained from an individual having a non-small cell lung cancer (NSCLC). In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations result in one or more of an insertion, deletion or substitution of one or more amino acid residues in a polypeptide encoded by the EGFR gene or the SMARCA4 gene. In some embodiments, the plurality of sequence reads is obtained by sequencing, whole exome sequencing, whole genome sequencing, gene-targeted sequencing, or next-generation sequencing.

In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a tumor mutational burden (TMB) of at least about 10 mut/Mb or at least about 20 mut/Mb, or one or more mutations in an RB1 gene, a KRAS gene, or a TP53 gene; and (c) detect, based on the analyzing, a TMB of at least about 10 mut/Mb or at least about 20 mut/Mb, or one or more mutations in an RB1 gene, a KRAS gene, or a TP53 gene in the sample. In some embodiments, the sample is obtained from an individual having a non-small cell lung cancer (NSCLC). In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations result in one or more of an insertion, deletion or substitution of one or more amino acid residues in a polypeptide encoded by the RB1 gene, KRAS gene, or TP53 gene. In some embodiments, the analyzing the plurality of sequence reads for the presence of a TMB of at least about 10 mut/Mb or at least about 20 mut/Mb is based on about 0.80 Mb of sequenced DNA. In some embodiments, the analyzing the plurality of sequence reads for the presence of a TMB of at least about 10 mut/Mb or at least about 20 mut/Mb is based on between about 0.83 Mb and about 1.14 Mb of sequenced DNA. In some embodiments, the analyzing the plurality of sequence reads for the presence of a TMB of at least about 10 mut/Mb or at least about 20 mut/Mb is based on about 1.1 Mb of sequenced DNA. In some embodiments, the analyzing the plurality of sequence reads for the presence of a TMB of at least about 10 mut/Mb or at least about 20 mut/Mb is based on up to about 1.1 Mb of sequenced DNA. In some embodiments, the plurality of sequence reads is obtained by sequencing, whole exome sequencing, whole genome sequencing, gene-targeted sequencing, or next-generation sequencing.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a tumor mutational burden (TMB) of at least about 10 mut/Mb or at least about 20 mut/Mb, or one or more mutations in an RB1 gene, a KRAS gene, or a TP53 gene; and (c) detecting, using the one or more processors and based on the analyzing, TMB of at least about 10 mut/Mb or at least about 20 mut/Mb, or one or more mutations in an RB1 gene, a KRAS gene, or a TP53 gene, in the sample. In some embodiments, the sample is obtained from an individual having a non-small cell lung cancer (NSCLC). In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations result in one or more of an insertion, deletion or substitution of one or more amino acid residues in a polypeptide encoded by the RB1 gene, KRAS gene, or TP53 gene. In some embodiments, the analyzing the plurality of sequence reads for the presence of a tumor mutational burden (TMB) of at least about 10 mut/Mb or at least about 20 mut/Mb is based on about 0.80 Mb of sequenced DNA. In some embodiments, the analyzing the plurality of sequence reads for the presence of a TMB of at least about 10 mut/Mb or at least about 20 mut/Mb is based on between about 0.83 Mb and about 1.14 Mb of sequenced DNA. In some embodiments, the analyzing the plurality of sequence reads for the presence of a TMB of at least about 10 mut/Mb or at least about 20 mut/Mb is based on about 1.1 Mb of sequenced DNA. In some embodiments, the analyzing the plurality of sequence reads for the presence of a TMB of at least about 10 mut/Mb or at least about 20 mut/Mb is based on up to about 1.1 Mb of sequenced DNA. In some embodiments, the plurality of sequence reads is obtained by sequencing, whole exome sequencing, whole genome sequencing, gene-targeted sequencing, or next-generation sequencing.

In another aspect, provided herein is an EGFR-targeted therapy for use in a method of treating or delaying progression of a NSCLC, wherein the method comprises administering an EGFR-targeted therapy to an individual having a NSCLC, wherein a deletion of an MTAP gene, or of a portion thereof, and one or more mutations in an EGFR gene are detected in a sample obtained from the individual.

In another aspect, provided herein is a SMARCA4-targeted therapy for use in a method of treating or delaying progression of a NSCLC, wherein the method comprises administering a SMARCA4-targeted therapy to an individual having a NSCLC, wherein a deletion of an MTAP gene, or of a portion thereof, and one or more mutations in a SMARCA4 gene are detected in a sample obtained from the individual.

In another aspect, provided herein is an immunotherapy for use in a method of treating or delaying progression of a NSCLC, wherein the method comprises administering an immunotherapy to an individual having a NSCLC, wherein a TMB of at least about 10 mut/Mb or at least about 20 mut/Mb, and/or a PD-L1-positive NSCLC are detected in a sample obtained from the individual. In some embodiments, the absence of a deletion of an MTAP gene, or of a portion thereof, is further detected in the sample.

In another aspect, provided herein is an RB1-targeted therapy for use in a method of treating or delaying progression of a NSCLC, wherein the method comprises administering an RB1-targeted therapy to an individual having a NSCLC, wherein one or more mutations in an RB1 gene are detected in a sample obtained from the individual. In some embodiments, the absence of a deletion of an MTAP gene, or of a portion thereof, is further detected in the sample.

In another aspect, provided herein is a KRAS-targeted therapy for use in a method of treating or delaying progression of a NSCLC, wherein the method comprises administering a KRAS-targeted therapy to an individual having a NSCLC, wherein one or more mutations in a KRAS gene are detected in a sample obtained from the individual. In some embodiments, the absence of a deletion of an MTAP gene, or of a portion thereof, is further detected in the sample.

In another aspect, provided herein is a TP53-targeted therapy for use in a method of treating or delaying progression of a NSCLC, wherein the method comprises administering a TP53-targeted therapy to an individual having a NSCLC, wherein one or more mutations in a TP53 gene are detected in a sample obtained from the individual. In some embodiments, the absence of a deletion of an MTAP gene, or of a portion thereof, is further detected in the sample.

In another aspect, provided herein is an EGFR-targeted therapy for use in the manufacture of a medicament for treating or delaying progression of a NSCLC, wherein the medicament is to be administered to an individual having a NSCLC, wherein a deletion of an MTAP gene, or of a portion thereof, and one or more mutations in an EGFR gene are detected in a sample obtained from the individual.

In another aspect, provided herein is a SMARCA4-targeted therapy for use in the manufacture of a medicament for treating or delaying progression of a NSCLC, wherein the medicament is to be administered to an individual having a NSCLC, wherein a deletion of an MTAP gene, or of a portion thereof, and one or more mutations in a SMARCA4 gene are detected in a sample obtained from the individual.

In another aspect, provided herein is an immunotherapy for use in the manufacture of a medicament for treating or delaying progression of a NSCLC, wherein the medicament is to be administered to an individual having a NSCLC, wherein a TMB of at least about 10 mut/Mb or at least about 20 mut/Mb, and/or a PD-L1-positive NSCLC are detected in a sample obtained from the individual. In some embodiments, the absence of a deletion of an MTAP gene, or of a portion thereof, is further detected in the sample.

In another aspect, provided herein is an RB1-targeted therapy for use in the manufacture of a medicament for treating or delaying progression of a NSCLC, wherein the medicament is to be administered to an individual having a NSCLC, wherein one or more mutations in an RB1 gene are detected in a sample obtained from the individual. In some embodiments, the absence of a deletion of an MTAP gene, or of a portion thereof, is further detected in the sample.

In another aspect, provided herein is a KRAS-targeted therapy for use in the manufacture of a medicament for treating or delaying progression of a NSCLC, wherein the medicament is to be administered to an individual having a NSCLC, wherein one or more mutations in a KRAS gene are detected in a sample obtained from the individual. In some embodiments, the absence of a deletion of an MTAP gene, or of a portion thereof, is further detected in the sample.

In another aspect, provided herein is a TP53-targeted therapy for use in the manufacture of a medicament for treating or delaying progression of a NSCLC, wherein the medicament is to be administered to an individual having a NSCLC, wherein one or more mutations in a TP53 gene are detected in a sample obtained from the individual. In some embodiments, the absence of a deletion of an MTAP gene, or of a portion thereof, is further detected in the sample.

In another aspect, provided herein is a method of identifying an individual having a sarcomatoid renal cell carcinoma (srcRCC) or a clear cell renal cell carcinoma (ccRCC) who may benefit from a treatment comprising a neurofibromatosis type II (NF2)-targeted therapy, the method comprising detecting in a sample from the individual a deletion of a methylthioadenosine phosphorylase (MTAP) gene, or of a portion thereof, and one or more mutations in an NF2 gene, wherein the presence of the deletion of the MTAP gene, or of the portion thereof, and one or more mutations in the NF2 gene in the sample identifies the individual as one who may benefit from a treatment comprising an NF2-targeted therapy.

In another aspect, provided herein is a method of selecting a treatment for an individual having a srcRCC or a ccRCC, the method comprising detecting a deletion of an MTAP gene, or of a portion thereof, and one or more mutations in an NF2 gene in a sample from the individual, wherein the presence of the deletion of the MTAP gene, or of the portion thereof, and one or more mutations in the NF2 gene in the sample identifies the individual as one who may benefit from a treatment comprising an NF2-targeted therapy.

In some embodiments, the presence in the sample of the deletion of the MTAP gene, or of the portion thereof, and the one or more mutations in the NF2 gene identifies the individual as one who may benefit from an additional treatment comprising a protein arginine methyltransferase 5 (PRMT5)-targeted therapy.

In another aspect, provided herein is a method of identifying one or more treatment options for an individual having a srcRCC or a ccRCC, the method comprising: (a) detecting a deletion of an MTAP gene, or of a portion thereof, and one or more mutations in an NF2 gene in a sample from the individual; and (b) generating a report, wherein the report comprises one or more treatment options identified for the individual based at least in part on the presence of the deletion of the MTAP gene, or of the portion thereof, and one or more mutations in the NF2 gene in the sample, wherein the one or more treatment options comprise an NF2-targeted therapy.

In another aspect, provided herein is a method of identifying one or more treatment options for an individual having a srcRCC or a ccRCC, the method comprising acquiring knowledge of a deletion of an MTAP gene, or of a portion thereof, and one or more mutations in an NF2 gene in a sample from the individual; and generating a report comprising one or more treatment options identified for the individual based at least in part on the knowledge of a deletion of the MTAP gene, or of a portion thereof, and one or more mutations in the NF2 gene in the sample, wherein the one or more treatment options comprise an NF2-targeted therapy.

In some embodiments, the one or more treatment options identified for the individual further comprise a PRMT5-targeted therapy.

In another aspect, provided herein is a method of selecting a treatment for an individual having a srcRCC or a ccRCC, comprising acquiring knowledge of a deletion of an MTAP gene, or of a portion thereof, and one or more mutations in an NF2 gene in a sample from the individual, wherein responsive to the acquisition of knowledge of a deletion of an MTAP gene, or of a portion thereof, and one or more mutations in an NF2 gene in the sample: (i) the individual is classified as a candidate to receive a treatment comprising an NF2-targeted therapy; and/or (ii) the individual is identified as likely to respond to a treatment that comprises an NF2-targeted therapy.

In some embodiments, the treatment further comprises a PRMT5-targeted therapy.

In another aspect, provided herein is a method of predicting survival of an individual having a srcRCC or a ccRCC treated with a treatment comprising an NF2-targeted therapy, the method comprising acquiring knowledge of a deletion of an MTAP gene, or of a portion thereof, and one or more mutations in an NF2 gene in a sample from the individual, wherein responsive to the acquisition of said knowledge, the individual is predicted to have longer survival after treatment with the NF2-targeted therapy, as compared to an individual whose srcRCC or ccRCC does not exhibit a deletion of an MTAP gene, or of a portion thereof, and one or more mutations in an NF2 gene.

In another aspect, provided herein is a method of treating or delaying progression of a srcRCC or a ccRCC, comprising acquiring knowledge of a deletion of an MTAP gene, or of a portion thereof, and one or more mutations in an NF2 gene in a sample from an individual, and responsive to said knowledge, administering to the individual an effective amount of a treatment comprising an NF2-targeted therapy.

In another aspect, provided herein is a method of treating or delaying progression of a srcRCC or a ccRCC, comprising responsive to acquiring knowledge of a deletion of an MTAP gene, or of a portion thereof, and one or more mutations in an NF2 gene in a sample from an individual, administering to the individual an effective amount of a treatment comprising an NF2-targeted therapy.

In some embodiments, the acquiring knowledge of the deletion of the MTAP gene, or of the portion thereof, and one or more mutations in the NF2 gene comprises detecting the deletion of the MTAP gene, or of the portion thereof, and the one or more mutations in the NF2 gene in the sample.

In another aspect, provided herein is a method of treating or delaying progression of a srcRCC or a ccRCC, comprising detecting a deletion of an MTAP gene, or of a portion thereof, and one or more mutations in an NF2 gene in a sample from an individual, and administering to the individual an effective amount of a treatment comprising an NF2-targeted therapy.

In another aspect, provided herein is a method of detecting the presence or absence of a srcRCC or a ccRCC in an individual, comprising: (a) detecting the presence or absence of a srcRCC or a ccRCC in a sample from an individual; and (b) detecting the presence or absence of a deletion of an MTAP gene, or of a portion thereof, and one or more mutations in an NF2 gene in the sample.

In another aspect, provided herein is a method of assessing a srcRCC or a ccRCC in an individual, the method comprising: (a) detecting a deletion of an MTAP gene, or of a portion thereof, and one or more mutations in an NF2 gene in a sample from the individual; and (b) providing an assessment of the deletion of the MTAP gene, or of the portion thereof, and the one or more mutations in the NF2 gene.

In another aspect, provided herein is a method of detecting a deletion of an MTAP gene, or of a portion thereof, and one or more mutations in an NF2 gene, the method comprising detecting the deletion of the MTAP gene, or of the portion thereof, and the one or more mutations in the NF2 gene in a sample from an individual having a srcRCC or a ccRCC.

In another aspect, provided herein is a method of detecting a deletion of an MTAP gene, or of a portion thereof, and one or more mutations in an NF2 gene, the method comprising: (a) providing a sample from an individual having a srcRCC or a ccRCC, wherein the sample comprises one or more nucleic acids; (b) preparing a nucleic acid sequencing library from the one or more nucleic acids in the sample; (c) amplifying said library using a polymerase chain reaction (PCR); (d) selectively enriching for one or more nucleic acids comprising MTAP or NF2 gene nucleotide sequences in said library to produce an enriched sample; (e) sequencing the enriched sample, thereby producing a plurality of sequencing reads; (f) analyzing the plurality of sequencing reads for the presence of a deletion of an MTAP gene, or of a portion thereof, and one or more mutations in an NF2 gene; (g) detecting, based on the analyzing step, a deletion of an MTAP gene, or of a portion thereof, and one or more mutations in an NF2 gene in the sample from the individual.

In some embodiments, the methods further comprise selectively enriching for one or more of: (a) one or more nucleic acids comprising nucleotide sequences that comprise the deletion of the MTAP gene, or of the portion thereof, or (b) one or more nucleic acids comprising nucleotide sequences that comprise the one or more mutations in the NF2 gene; wherein the selectively enriching produces an enriched sample.

In another aspect, provided herein is a method of treating or delaying progression of a srcRCC or a ccRCC, comprising administering to an individual having a srcRCC or a ccRCC an effective amount of a treatment comprising an NF2-targeted therapy, wherein the srcRCC or ccRCC comprises a deletion of an MTAP gene, or of a portion thereof, and one or more mutations in an NF2 gene.

In some embodiments, the methods further comprise acquiring knowledge of or detecting in the sample a deletion of a CDKN2A gene, or of a portion thereof; and/or a deletion of a CDKN2B gene, or of a portion thereof. In some embodiments, the methods comprise acquiring knowledge of or detecting in the sample one or more mutations in one or more genes, wherein the one or more genes comprise VHL, PBRM1, SETD2, BAP1, TP53, PTEN, TERT, TSC1, PIK3CA, ATM, or MET. In some embodiments, the individual has one or more mutations in one or more genes, wherein the one or more genes comprise VHL, PBRM1, SETD2, BAP1, TP53, PTEN, TERT, TSC1, PIK3CA, ATM, or MET. In some embodiments, the methods comprise administering to the individual an effective amount of a treatment comprising an anti-cancer therapy. In some embodiments, the methods further comprise selectively enriching for one or more nucleic acids comprising nucleotide sequences that comprise one or more mutations in one or more genes, wherein the one or more genes comprise VHL, PBRM1, SETD2, BAP1, TP53, PTEN, TERT, TSC1, PIK3CA, ATM, or MET, to produce an enriched sample. In some embodiments, the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

In some embodiments, the individual has a deletion of a CDKN2A gene, or of a portion thereof; and/or a deletion of a CDKN2B gene, or of a portion thereof. In some embodiments, the individual has a heterozygous or a homozygous deletion of a CDKN2A gene, or of a portion thereof; and/or a heterozygous or a homozygous deletion of a CDKN2B gene, or of a portion thereof. In some embodiments, the individual has a heterozygous or a homozygous deletion of the MTAP gene, or of a portion thereof.

In some embodiments, the methods further comprise administering to the individual a PRMT5-targeted therapy. In some embodiments, the individual is being treated with a PRMT5-targeted therapy. In some embodiments, the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

In some embodiments, the one or more mutations in the NF2 gene comprise one or more of an insertion, deletion or substitution of one or more nucleotides, a genomic rearrangement, an alteration in a promoter, a gene fusion, or a copy number alteration. In some embodiments, the one or more mutations in the NF2 gene result in one or more of an insertion, deletion or substitution of one or more amino acid residues in a polypeptide encoded by the NF2 gene.

In some embodiments, the NF2-targeted therapy is a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the NF2-targeted therapy comprises one or more of an mTOR inhibitor, a VEGF inhibitor, a focal adhesion kinase (FAK) inhibitor, an EGFR inhibitor, a NEDD8-activating enzyme (NAE) inhibitor, a MET inhibitor, a MEK inhibitor, a SRC inhibitor, a JNK inhibitor, a CDK inhibitor, a WEE1, a CHK1 inhibitor, or a multi-targeted kinase inhibitor.

In some embodiments, the deletion of the MTAP gene or of the portion thereof, or the one or more mutations in the NF2 gene are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the deletion of the MTAP gene or of the portion thereof, or the one or more mutations in the NF2 gene are detected in a polypeptide encoded by the MTAP gene, or the NF2 gene. In some embodiments, the deletion of the MTAP gene or of the portion thereof, or the one or more mutations in the NF2 gene are detected in the sample by one or more of: immunoblotting, enzyme linked immunosorbent assay (ELISA), immunohistochemistry, or mass spectrometry. In some embodiments, the deletion of the CDKN2A gene or of the portion thereof, and/or the deletion of the CDKN2B gene or of the portion thereof, are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the deletion of the CDKN2A gene or of the portion thereof, and/or the deletion of the CDKN2B gene or of the portion thereof, are detected in a polypeptide encoded by the CDKN2A gene or the CDKN2B gene. In some embodiments, the deletion of the CDKN2A gene or of the portion thereof, and/or the deletion of the CDKN2B gene or of the portion thereof, are detected in the sample by one or more of: immunoblotting, enzyme linked immunosorbent assay (ELISA), immunohistochemistry, or mass spectrometry.

In another aspect, provided herein is a method of identifying an individual having a clear cell renal cell carcinoma (ccRCC) who may benefit from a treatment comprising a Von Hippel-Lindau tumor suppressor (VHL)-targeted therapy, or a protein polybromo-1 (PBRM1)-targeted therapy, the method comprising detecting in a sample from the individual one or more mutations in a VHL gene or a PBRM1 gene; wherein: (i) the presence in the sample of one or more mutations in the VHL gene identifies the individual as one who may benefit from a treatment comprising a VHL-targeted therapy; or (ii) the presence in the sample of one or more mutations in the PBRM1 gene identifies the individual as one who may benefit from a treatment comprising a PBRM1-targeted therapy.

In another aspect, provided herein is a method of selecting a treatment for an individual having a ccRCC, the method comprising detecting one or more mutations in a VHL gene or a PBRM1 gene, in a sample from the individual; wherein: (i) the presence in the sample of one or more mutations in the VHL gene identifies the individual as one who may benefit from a treatment comprising a VHL-targeted therapy; or (ii) the presence in the sample of one or more mutations in the PBRM1 gene identifies the individual as one who may benefit from a treatment comprising a PBRM1-targeted therapy.

In another aspect, provided herein is a method of identifying one or more treatment options for an individual having a ccRCC, the method comprising: (a) detecting in a sample from the individual one or more mutations in a VHL gene or a PBRM1 gene; and (b) generating a report, wherein the report comprises: (i) one or more treatment options identified for the individual based at least in part on the presence of one or more mutations in the VHL gene in the sample, wherein the one or more treatment options comprise a VHL-targeted therapy; (ii) one or more treatment options identified for the individual based at least in part on the presence of one or more mutations in the PBRM1 gene in the sample, wherein the one or more treatment options comprise a PBRM1-targeted therapy.

In another aspect, provided herein is a method of identifying one or more treatment options for an individual having a ccRCC, the method comprising acquiring knowledge of one or more mutations in a VHL gene or a PBRM1 gene, in a sample from the individual; and generating a report comprising: (i) one or more treatment options identified for the individual based at least in part on the knowledge of one or more mutations in the VHL gene in the sample, wherein the one or more treatment options comprise a VHL-targeted therapy; or (ii) one or more treatment options identified for the individual based at least in part on the knowledge of one or more mutations in the PBRM1 gene in the sample, wherein the one or more treatment options comprise a PBRM1-targeted therapy.

In another aspect, provided herein is a method of selecting a treatment for an individual having a ccRCC, comprising acquiring knowledge of one or more mutations in a VHL gene or a PBRM1 gene, in a sample from the individual, wherein: (a) responsive to acquisition of knowledge of one or more mutations in the VHL gene in the sample: (i) the individual is classified as a candidate to receive a treatment comprising a VHL-targeted therapy; and/or (ii) the individual is identified as likely to respond to a treatment that comprises a VHL-targeted therapy; or (b) responsive to acquisition of knowledge of one or more mutations in the PBRM1 gene in the sample: (i) the individual is classified as a candidate to receive a treatment comprising a PBRM1-targeted therapy; and/or (ii) the individual is identified as likely to respond to a treatment that comprises a PBRM1-targeted therapy.

In another aspect, provided herein is a method of predicting survival of an individual having a ccRCC treated with a treatment comprising a VHL-targeted therapy or a PBRM1-targeted therapy, the method comprising acquiring knowledge of one or more mutations in a VHL gene or a PBRM1 gene, in a sample from the individual; wherein responsive to acquisition of said knowledge, the individual is predicted to have longer survival after treatment with the VHL-targeted therapy or PBRM1-targeted therapy, as compared to an individual whose ccRCC does not exhibit one or more mutations in the VHL gene or PBRM1 gene.

In another aspect, provided herein is a method of treating or delaying progression of a ccRCC, comprising: (a) acquiring knowledge of one or more mutations in a VHL gene in a sample from an individual, and responsive to said knowledge, administering to the individual an effective amount of a treatment comprising a VHL-targeted therapy; or (b) acquiring knowledge of one or more mutations in a PBRM1 gene in a sample from an individual, and responsive to said knowledge, administering to the individual an effective amount of a treatment comprising a PBRM1-targeted therapy.

In another aspect, provided herein is a method of treating or delaying progression of a ccRCC, comprising: (a) responsive to acquiring knowledge of one or more mutations in a VHL gene in a sample from an individual, administering to the individual an effective amount of a treatment comprising a VHL-targeted therapy; or (b) responsive to acquiring knowledge of one or more mutations in a PBRM1 gene in a sample from an individual, administering to the individual an effective amount of a treatment comprising a PBRM1-targeted therapy.

In some embodiments, acquiring knowledge of one or more mutations in a VHL gene or a PBRM1 gene comprises detecting the one or more mutations in the VHL gene or PBRM1 gene in the sample.

In another aspect, provided herein is a method of treating or delaying progression of a ccRCC, comprising: (a) detecting one or more mutations in a VHL gene in a sample from an individual, and administering to the individual an effective amount of a treatment comprising a VHL-targeted therapy; or (b) detecting one or more mutations in a PBRM1 gene in a sample from an individual, and administering to the individual an effective amount of a treatment comprising a PBRM1-targeted therapy.

In another aspect, provided herein is a method of detecting the presence or absence of a ccRCC in an individual, comprising: (a) detecting the presence or absence of a ccRCC in a sample from an individual; and (i) detecting the presence or absence of one or more mutations in a VHL gene in the sample, or (ii) detecting the presence or absence of one or more mutations in a PBRM1 gene in the sample.

In another aspect, provided herein is a method of assessing a ccRCC in an individual, the method comprising: (a) detecting one or more mutations in a VHL gene or a PBRM1 gene, in a sample from the individual; and (b) providing an assessment of the one or more mutations in the VHL gene or PBRM1 gene in the sample.

In another aspect, provided herein is a method of detecting one or more mutations in a VHL gene or a PBRM1 gene, the method comprising detecting the one or more mutations in the VHL gene or PBRM1 gene in a sample from an individual having a ccRCC.

In another aspect, provided herein is a method of detecting one or more mutations in a VHL gene or a PBRM1 gene, the method comprising: (a) providing a sample from an individual having a ccRCC, wherein the sample comprises one or more nucleic acids; (b) preparing a nucleic acid sequencing library from the one or more nucleic acids in the sample; (c) amplifying said library using a polymerase chain reaction (PCR); (d) selectively enriching for one or more nucleic acids comprising VHL gene or PBRM1 gene nucleotide sequences in said library to produce an enriched sample; (e) sequencing the enriched sample, thereby producing a plurality of sequencing reads; (f) analyzing the plurality of sequencing reads for the presence of one or more mutations in a VHL gene or a PBRM1 gene; (g) detecting, based on the analyzing step, one or more mutations in a VHL gene or a PBRM1 gene in the sample from the individual.

In another aspect, provided herein is a method of treating or delaying progression of a ccRCC, comprising: (a) administering to an individual having a ccRCC an effective amount of a treatment comprising a VHL-targeted therapy, wherein the ccRCC comprises one or more mutations in a VHL gene; or (b) administering to an individual having a ccRCC an effective amount of a treatment comprising a PBRM1-targeted therapy, wherein the ccRCC comprises one or more mutations in a PBRM1 gene.

In some embodiments, the methods further comprise acquiring knowledge of the absence of a deletion of an MTAP gene, or of a portion thereof, in the sample. In some embodiments, acquiring knowledge of the absence of a deletion of an MTAP gene, or of a portion thereof, comprises detecting the absence of a deletion of an MTAP gene, or of a portion thereof, in the sample. In some embodiments, the methods further comprise detecting the absence of a deletion of an MTAP gene, or of a portion thereof, in the sample.

In some embodiments, the methods comprise acquiring knowledge of or detecting in the sample one or more mutations in one or more genes, wherein the one or more genes comprise SETD2, BAP1, TP53, PTEN, TERT, TSC1, PIK3CA, ATM, NF2, or MET. In some embodiments, the individual has one or more mutations in one or more genes, wherein the one or more genes comprise SETD2, BAP1, TP53, PTEN, TERT, TSC1, PIK3CA, ATM, NF2, or MET. In some embodiments, the methods further comprise administering to the individual an effective amount of a treatment comprising anti-cancer therapy. In some embodiments, the methods comprise selectively enriching for one or more nucleic acids comprising nucleotide sequences that comprise one or more mutations in one or more genes to produce an enriched sample, wherein the one or more genes comprise SETD2, BAP1, TP53, PTEN, TERT, TSC1, PIK3CA, ATM, NF2, or MET. In some embodiments, the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the one or more mutations in the one or more genes are detected in a polypeptide encoded by the one or more genes. In some embodiments, the one or more mutations in the one or more genes are detected in the sample by one or more of immunoblotting, enzyme linked immunosorbent assay (ELISA), immunohistochemistry, or mass spectrometry.

In some embodiments, the methods further comprise selectively enriching for one or more nucleic acids comprising MTAP nucleotide sequences to produce an enriched sample. In some embodiments, the absence of a deletion of an MTAP gene, or of a portion thereof, is detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the individual does not have a deletion of an MTAP gene, or of a portion thereof.

In some embodiments, the methods further comprise selectively enriching for one or more nucleic acids comprising nucleotide sequences that comprise the one or more mutations in the VHL gene or PBRM1 gene to produce an enriched sample. In some embodiments, the one or more mutations in the VHL gene or PBRM1 gene comprise one or more of an insertion, deletion or substitution of one or more nucleotides, a genomic rearrangement, an alteration in a promoter of the VHL gene or PBRM1 gene, a gene fusion, or a copy number alteration. In some embodiments, the one or more mutations in the VHL gene or PBRM1 gene result in one or more of an insertion, deletion or substitution of one or more amino acid residues in a polypeptide encoded by the VHL gene or PBRM1 gene. In some embodiments, the one or more mutations in the VHL gene or PBRM1 gene are detected in the sample by one or more of: an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the one or more mutations in the VHL gene or PBRM1 gene are detected in a polypeptide encoded by the VHL gene or PBRM1 gene. In some embodiments, the one or more mutations in the VHL gene or PBRM1 gene are detected in the sample by one or more of immunoblotting, enzyme linked immunosorbent assay (ELISA), immunohistochemistry, or mass spectrometry.

In some embodiments, the VHL-targeted therapy or the PBRM1-targeted therapy is a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the VHL-targeted therapy comprises one or more of a poly(ADP-ribose) polymerase (PARP) inhibitor, a glutaminase 1 (GLS-1) inhibitor, a VEGF inhibitor, a HIF-2alpha inhibitor, an HDAC inhibitor, a CDK4/6 inhibitor, a Tank binding kinase 1 (TBK1) inhibitor, an EZH1 and/or EZH2 inhibitor, a Rho-Associated Kinase 1 (ROCK1) inhibitor, a glucose transporter 1 (GLUT1) inhibitor, an autophagy modulator, or an immune checkpoint inhibitor; or the PBRM1-targeted therapy comprises one or more of an EZH2 inhibitor, a VEGF inhibitor, or an mTOR inhibitor.

In some embodiments, the srcRCC or ccRCC is advanced stage srcRCC or ccRCC and/or refractory srcRCC or ccRCC. In some embodiments, the ccRCC is advanced stage ccRCC and/or refractory ccRCC. In some embodiments, the srcRCC or ccRCC is microsatellite stable and/or has a low tumor mutational burden (TMB). In some embodiments, the ccRCC is microsatellite stable and/or has a low tumor mutational burden (TMB).

In some embodiments, the treatment or the one or more treatment options further comprise an additional anti-cancer treatment. In some embodiments, the additional anti-cancer treatment comprises one or more of a small molecule inhibitor, an antibody, a cellular therapy, or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the additional anti-cancer treatment comprises one or more of a surgery, radiotherapy, chemotherapy, anti-angiogenic therapy, anti-DNA repair therapy, anti-inflammatory therapy, an anti-neoplastic agent, a chemotherapeutic agent, a growth inhibitory agent, an anti-angiogenic agent, or a cytotoxic agent.

In some embodiments, the sample from the individual comprises fluid, cells, or tissue. In some embodiments, the sample from the individual comprises a tumor biopsy or a circulating tumor cell. In some embodiments, the sample from the individual comprises one or more nucleic acids. In some embodiments, the sample from the individual comprises mRNA, genomic DNA, circulating tumor DNA, cell-free DNA, or cell-free RNA. In some embodiments, the sample from the individual comprises one or more polypeptides. In some embodiments, the sample is a formalin-fixed paraffin-embedded (FFPE) sample. In some embodiments, the sample comprises one or more nucleic acids and/or one or more polypeptides obtained from an FFPE sample from the individual. In some embodiments, the one or more nucleic acids comprise mRNA and/or genomic DNA. In some embodiments, the sample is derived from a srcRCC or a ccRCC in the individual.

In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the bait comprises a capture nucleic acid molecule configured to hybridize to the one or more nucleic acids. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and 200 nucleotides. In some embodiments, the bait is conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample.

In another aspect, provided herein is a system comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a deletion of a methylthioadenosine phosphorylase (MTAP) gene, or of a portion thereof, and one or more mutations in an NF2 gene; and (c) detect, based on the analyzing, a deletion of an MTAP gene, or of a portion thereof, and one or more mutations in an NF2 gene in the sample. In some embodiments, the sample is obtained from an individual having a srcRCC or a ccRCC. In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations in the NF2 gene result in one or more of an insertion, deletion or substitution of one or more amino acid residues in a polypeptide encoded by the NF2 gene. In some embodiments, the plurality of sequence reads is obtained by sequencing, whole exome sequencing, whole genome sequencing, gene-targeted sequencing, or next-generation sequencing.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a deletion of a methylthioadenosine phosphorylase (MTAP) gene, or of a portion thereof, and one or more mutations in an NF2 gene; and (c) detecting, using the one or more processors and based on the analyzing, a deletion of an MTAP gene, or of a portion thereof, and one or more mutations in an NF2 gene, in the sample. In some embodiments, the sample is obtained from an individual having a srcRCC or a ccRCC. In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations in the NF2 gene result in one or more of an insertion, deletion or substitution of one or more amino acid residues in a polypeptide encoded by the NF2 gene. In some embodiments, the plurality of sequence reads is obtained by sequencing, whole exome sequencing, whole genome sequencing, gene-targeted sequencing, or next-generation sequencing.

In another aspect, provided herein is a system comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of one or more mutations in a VHL gene or a PBRM1 gene; and (c) detect, based on the analyzing, one or more mutations in a VHL gene or a PBRM1 gene in the sample. In some embodiments, the sample is obtained from an individual having a ccRCC. In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations in the VHL gene or PBRM1 gene result in one or more of an insertion, deletion or substitution of one or more amino acid residues in a polypeptide encoded by the VHL gene or PBRM1 gene. In some embodiments, the plurality of sequence reads is obtained by sequencing, whole exome sequencing, whole genome sequencing, gene-targeted sequencing, or next-generation sequencing.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of one or more mutations in a VHL gene or a PBRM1 gene; and (c) detecting, using the one or more processors and based on the analyzing, one or more mutations in a VHL gene or a PBRM1 gene, in the sample. In some embodiments, the sample is obtained from an individual having a ccRCC. In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations in the VHL gene or PBRM1 gene result in one or more of an insertion, deletion or substitution of one or more amino acid residues in a polypeptide encoded by the VHL gene or PBRM1 gene. In some embodiments, the plurality of sequence reads is obtained by sequencing, whole exome sequencing, whole genome sequencing, gene-targeted sequencing, or next-generation sequencing.

In another aspect, provided herein is an NF2-targeted therapy for use in a method of treating or delaying progression of a srcRCC or a ccRCC, wherein the method comprises administering an NF2-targeted therapy to an individual having a srcRCC or a ccRCC, wherein a deletion of an MTAP gene, or of a portion thereof, and one or more mutations in an NF2 gene are detected in a sample obtained from the individual.

In another aspect, provided herein is a VHL-targeted therapy for use in a method of treating or delaying progression of a ccRCC, wherein the method comprises administering a VHL-targeted therapy to an individual having a ccRCC, wherein one or more mutations in a VHL gene are detected in a sample obtained from the individual. In some embodiments, the absence of a deletion of an MTAP gene, or of a portion thereof, is further detected in the sample.

In another aspect, provided herein is a PBRM1-targeted therapy for use in a method of treating or delaying progression of a ccRCC, wherein the method comprises administering a PBRM1-targeted therapy to an individual having a ccRCC, wherein one or more mutations in a PBRM1 gene are detected in a sample obtained from the individual. In some embodiments, the absence of a deletion of an MTAP gene, or of a portion thereof, is further detected in the sample.

In another aspect, provided herein is an NF2-targeted therapy for use in the manufacture of a medicament for treating or delaying progression of a srcRCC or a ccRCC, wherein the medicament is to be administered to an individual having a srcRCC or a ccRCC, wherein a deletion of an MTAP gene, or of a portion thereof, and one or more mutations in an NF2 gene are detected in a sample obtained from the individual.

In another aspect, provided herein is a VHL-targeted therapy for use in the manufacture of a medicament for treating or delaying progression of a ccRCC, wherein the medicament is to be administered to an individual having a ccRCC, wherein one or more mutations in a VHL gene are detected in a sample obtained from the individual. In some embodiments, the absence of a deletion of an MTAP gene, or of a portion thereof, is further detected in the sample.

In another aspect, provided herein is a PBRM1-targeted therapy for use in the manufacture of a medicament for treating or delaying progression of a ccRCC, wherein the medicament is to be administered to an individual having a ccRCC, wherein one or more mutations in a PBRM1 gene are detected in a sample obtained from the individual. In some embodiments, the absence of a deletion of an MTAP gene, or of a portion thereof, is further detected in the sample.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 is a schematic of the comprehensive genomic profiling (CGP) assay used in the study described in Example 1. FFPE=Formalin-fixed paraffin-embedded.

FIG. 2 is a schematic of MTAP and CDKN2A/B co-deletion on chromosome 9. MTAP deletion leads to build-up of the amino acids methionine and arginine.

FIGS. 3A-3B show long tail plots of genomic alterations identified in MTAP-intact (MTAP+) and MTAP-deleted (MTAP-) urothelial bladder cancer (UCB) samples analyzed as described in Example 1. FIG. 3A shows a long tail plot of genomic alterations identified in MTAP-UCB samples, while FIG. 3B shows a long tail plot of genomic alterations identified in MTAP+ UCB samples. The legend in FIGS. 3A-3B indicates the types of genomic alterations that were identified (short variant mutations, genomic copy number alterations, genomic rearrangements/fusions, and multiple alterations per sample).

FIGS. 4A-4D depict the results of CGP and histological analyses of a stage IV UCB in a 79-year old man. FIGS. 4A-4B show histology images of the stage IV UCB using standard hematoxylin and eosin stained tissue sections at low (FIG. 4A) and high magnification (FIG. 4B). FIG. 4C shows a co-deletion of MTAP, CDKN2A and CDKN2B identified in the UCB tumor. FIG. 4D shows an Integrative Genomics Viewer (IGV) view of an FGFR2-CASP7 gene fusion identified in the UCB tumor. The FGFR2-CASP7 fusion was a chromosome 10 (chr10) inversion fragment (5′-CASP7 (ex1-1 UTR NM_001227)-FGFR2 (ex18-18 NM_000141)), with breakpoints at CASP7 intron 1 and FGFR2 intron 17, and the inversion was reciprocal.

FIGS. 5A-5E depict the results of CGP and histological analyses of UCB in a 71-year old woman with metastatic disease. FIGS. 5A-5C show images of a transurethral bladder biopsy showing deeply invasive urothelial carcinoma using standard hematoxylin and eosin stained tissue sections at low and high magnification. FIG. 5D shows a co-deletion of MTAP, CDKN2A and CDKN2B identified in the patient. FIG. 5E shows an IGV view of an ERBB2 V777L kinase domain missense mutation identified in the patient.

FIG. 6 depicts an exemplary device, “Device 300,” in accordance with some embodiments of the disclosure.

FIG. 7 depicts an exemplary device, “Device 1100,” in accordance with some embodiments.

FIG. 8 depicts an exemplary system, “System 1200,” in accordance with some embodiments.

FIG. 9 depicts a block diagram of an exemplary process for detecting an MTAP deletion and one or more mutations in an FGFR3 gene or in a PTEN gene, in accordance with some embodiments.

FIG. 10 depicts a block diagram of an exemplary process for detecting a TMB of at least about 10 mut/Mb or at least about 20 mut/Mb, and/or one or more mutations in an RB1 gene, in accordance with some embodiments.

FIG. 11 depicts a block diagram of an exemplary process for detecting an MTAP deletion and one or more mutations in an EGFR gene or in a SMARCA4 gene, in accordance with some embodiments.

FIG. 12 depicts a block diagram of an exemplary process for detecting a TMB of at least about 10 mut/Mb or at least about 20 mut/Mb, and/or one or more mutations in an RB1 gene, a KRAS gene, or a TP53 gene, in accordance with some embodiments.

FIG. 13 depicts a block diagram of an exemplary process for detecting an MTAP deletion and one or more mutations in an NF2 gene, in accordance with some embodiments.

FIG. 14 depicts a block diagram of an exemplary process for detecting one or more mutations in a VHL gene or a PBRM1 gene, in accordance with some embodiments.

### DETAILED DESCRIPTION

The present disclosure is based, at least in part, on the discovery of certain biomarkers and their associations with urothelial bladder cancers (UCB), non-small cell lung cancers (NSCLC), sarcomatoid renal cell carcinomas (srcRCC), or clear cell renal cell carcinomas (ccRCC) having either an intact methylthioadenosine phosphorylase (MTAP) gene, or a deletion of an MTAP gene or of a portion thereof.

In some aspects, provided herein are methods of treating or delaying progression of a UCB. In other aspects, provided herein are methods of identifying one or more treatment options for an individual having a UCB. In other aspects, provided herein are methods of selecting treatment for an individual having a UCB. In other aspects, provided herein are methods of predicting survival of an individual having a UCB treated with an anti-cancer therapy. In other aspects, provided herein are methods of identifying an individual having a UCB who may benefit from a treatment comprising an anti-cancer therapy. In other aspects, provided herein are methods of selecting a therapy for an individual having a UCB.

In some embodiments, the UCB comprises a deletion of an MTAP gene, or of a portion thereof, and one or more mutations in a fibroblast growth factor receptor 3 (FGFR3) gene and/or in a phosphatase and tensin homolog (PTEN) gene. In some embodiments, the UCB comprises a deletion of an MTAP gene, or of a portion thereof, and one or more mutations in one or more genes, wherein the one or more genes comprise CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the methods comprise acquiring knowledge of a deletion of an MTAP gene, or of a portion thereof, and one or more mutations in an FGFR3 gene or in a PTEN gene in a sample from an individual. In some embodiments, the methods comprise acquiring knowledge of a deletion of an MTAP gene, or of a portion thereof, and one or more mutations in one or more genes, wherein the one or more genes comprise CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample from an individual. In some embodiments, the methods comprise detecting a deletion of an MTAP gene, or of a portion thereof, and one or more mutations in an FGFR3 gene or in a PTEN gene in a sample from an individual. In some embodiments, the methods comprise detecting a deletion of an MTAP gene, or of a portion thereof, and one or more mutations in one or more genes, wherein the one or more genes comprise CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample from an individual. In some embodiments, the methods comprise generating a report comprising one or more treatment options for the individual. In some embodiments, the methods comprise administering to an individual having a UCB an effective amount of a treatment comprising an anti-cancer therapy. In some embodiments, the UCB comprises a deletion of an MTAP gene, or of a portion thereof, and one or more mutations in an FGFR3 gene or in a PTEN gene described herein. In some embodiments, the UCB comprises a deletion of an MTAP gene, or of a portion thereof, and one or more mutations in one or more genes, wherein the one or more genes comprise CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the anti-cancer therapy is an anti-cancer therapy described herein. In some embodiments, the anti-cancer therapy is an FGFR-targeted therapy or a PTEN-targeted therapy. In some embodiments, the anti-cancer therapy is a PRMT5-targeted therapy. In some embodiments, the anti-cancer therapy is a combination therapy comprising an FGFR-targeted therapy or a PTEN-targeted therapy, and a PRMT5-targeted therapy.

In other aspects, provided herein are methods of detecting a deletion of an MTAP gene, or of a portion thereof, and one or more mutations in an FGFR3 gene or in a PTEN gene. In other aspects, provided herein are methods of detecting a deletion of an MTAP gene, or of a portion thereof, and one or more mutations in one or more genes, wherein the one or more genes comprise CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In other aspects, provided herein are methods of assessing a deletion of an MTAP gene, or of a portion thereof, and one or more mutations in an FGFR3 gene or in a PTEN gene. In other aspects, provided herein are methods of assessing a deletion of an MTAP gene, or of a portion thereof, and one or more mutations in one or more genes, wherein the one or more genes comprise CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1.

In some aspects, provided herein are methods of detecting the presence or absence of a UCB in an individual. In some embodiments, the methods comprise detecting the presence or absence of a UCB in a sample from an individual. In some embodiments, the methods comprise detecting the presence or absence of a deletion of an MTAP gene, or of a portion thereof, and one or more mutations in an FGFR3 gene in the sample; and/or detecting the presence or absence of a deletion of an MTAP gene, or of a portion thereof, and one or more mutations in a PTEN gene in the sample.

In other aspects, provided herein are systems comprising one or more processors; and a non-transitory computer readable storage medium comprising one or more programs executable by the one or more processors for performing a method. In some embodiments, the method comprises: (a) sequencing one or more nucleic acids, thereby producing a plurality of sequencing reads, wherein the one or more nucleic acids are derived from a sample obtained from an individual having a UCB; (b) analyzing the plurality of sequencing reads for the presence of a deletion of an MTAP gene, or of a portion thereof, and one or more mutations in an FGFR3 gene or in a PTEN gene; and (c) detecting, based on the analyzing step, a deletion of an MTAP gene, or of a portion thereof, and one or more mutations in an FGFR3 gene or in a PTEN gene in the sample. In some embodiments, the method comprises: (a) sequencing one or more nucleic acids, thereby producing a plurality of sequencing reads, wherein the one or more nucleic acids are derived from a sample obtained from an individual having a UCB; (b) analyzing the plurality of sequencing reads for the presence of a deletion of an MTAP gene, or of a portion thereof, and one or more mutations in one or more genes, wherein the one or more genes comprise CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1; and (c) detecting, based on the analyzing step, a deletion of an MTAP gene, or of a portion thereof, and one or more mutations in one or more genes, wherein the one or more genes comprise CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample.

In some embodiments, the UCB comprises an intact MTAP gene. In some embodiments, the UCB comprises a tumor mutational burden (TMB) of at least about 10 mut/Mb or at least about 20 mut/Mb, a PD-L1-positive status, and/or one or more mutations in an RB1 gene. In some embodiments, the UCB comprises a tumor mutational burden (TMB) of at least about 10 mut/Mb or at least about 20 mut/Mb, a PD-L1-positive status, and/or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the methods comprise acquiring knowledge of a TMB of at least about 10 mut/Mb or at least about 20 mut/Mb, a PD-L1-positive status, and/or one or more mutations in an RB1 gene in a sample from an individual. In some embodiments, the methods comprise acquiring knowledge of a TMB of at least about 10 mut/Mb or at least about 20 mut/Mb, a PD-L1-positive status, and/or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample from an individual. In some embodiments, the methods comprise detecting a TMB of at least about 10 mut/Mb or at least about 20 mut/Mb, a PD-L1-positive status, and/or one or more mutations in an RB1 gene in a sample from an individual. In some embodiments, the methods comprise detecting a TMB of at least about 10 mut/Mb or at least about 20 mut/Mb, a PD-L1-positive status, and/or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample from an individual. In some embodiments, the methods comprise generating a report comprising one or more treatment options for the individual. In some embodiments, the methods comprise administering to an individual having a UCB an effective amount of a treatment comprising an anti-cancer therapy. In some embodiments, the UCB comprises a TMB of at least about 10 mut/Mb or at least about 20 mut/Mb, a PD-L1-positive status, and/or one or more mutations in an RB1 gene. In some embodiments, the UCB comprises a TMB of at least about 10 mut/Mb or at least about 20 mut/Mb, a PD-L1-positive status, and/or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the anti-cancer therapy is an anti-cancer therapy described herein. In some embodiments, the anti-cancer therapy is an RB1-targeted therapy or an immunotherapy.

In other aspects, provided herein are methods of detecting a TMB of at least about 10 mut/Mb or at least about 20 mut/Mb, a PD-L1-positive status, and/or one or more mutations in an RB1 gene. In other aspects, provided herein are methods of detecting a TMB of at least about 10 mut/Mb or at least about 20 mut/Mb, a PD-L1-positive status, and/or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In other aspects, provided herein are methods of assessing a TMB of at least about 10 mut/Mb or at least about 20 mut/Mb, a PD-L1-positive status, and/or one or more mutations in an RB1 gene. In other aspects, provided herein are methods of assessing a TMB of at least about 10 mut/Mb or at least about 20 mut/Mb, a PD-L1-positive status, and/or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2.

In some aspects, provided herein are methods of detecting the presence or absence of a UCB in an individual. In some embodiments, the methods comprise detecting the presence or absence of a UCB in a sample from an individual. In some embodiments, the methods comprise detecting the presence or absence of a TMB of at least about 10 mut/Mb or at least about 20 mut/Mb, or a PD-L1-positive UCB in the sample; and/or detecting the presence or absence of one or more mutations in an RB1 gene in the sample.

In other aspects provided herein are systems comprising one or more processors; and a non-transitory computer readable storage medium comprising one or more programs executable by the one or more processors for performing a method. In some embodiments, the method comprises: (a) sequencing one or more nucleic acids, thereby producing a plurality of sequencing reads, wherein the one or more nucleic acids are derived from a sample obtained from an individual having a UCB; (b) analyzing the plurality of sequencing reads for the presence of a TMB of at least about 10 mut/Mb or at least about 20 mut/Mb, and/or one or more mutations in an RB1 gene; and (c) detecting, based on the analyzing step, a TMB of at least about 10 mut/Mb or at least about 20 mut/Mb, and/or one or more mutations in an RB1 gene in the sample. In some embodiments, the method comprises: (a) sequencing one or more nucleic acids, thereby producing a plurality of sequencing reads, wherein the one or more nucleic acids are derived from a sample obtained from an individual having a UCB; (b) analyzing the plurality of sequencing reads for the presence of a TMB of at least about 10 mut/Mb or at least about 20 mut/Mb, and/or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2; and (c) detecting, based on the analyzing step, a TMB of at least about 10 mut/Mb or at least about 20 mut/Mb, and/or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample.

In some embodiments, the individual is a human.

In other aspects, provided herein is an FGFR-targeted therapy for use in a method of treating or delaying progression of a UCB, wherein the method comprises administering an FGFR-targeted therapy to an individual having a UCB, wherein a deletion of an MTAP gene, or of a portion thereof, and one or more mutations in an FGFR3 gene are detected in a sample obtained from the individual.

In other aspects, provided herein is a PTEN-targeted therapy for use in a method of treating or delaying progression of a UCB, wherein the method comprises administering a PTEN-targeted therapy to an individual having a UCB, wherein a deletion of an MTAP gene, or of a portion thereof, and one or more mutations in a PTEN gene are detected in a sample obtained from the individual.

In other aspects, provided herein is an immunotherapy for use in a method of treating or delaying progression of a UCB, wherein the method comprises administering an immunotherapy to an individual having a UCB, wherein a TMB of at least about 10 mut/Mb or at least about 20 mut/Mb, and/or a PD-L1-positive UCB are detected in a sample obtained from the individual.

In other aspects, provided herein is an RB1-targeted therapy for use in a method of treating or delaying progression of a UCB, wherein the method comprises administering an RB1-targeted therapy to an individual having a UCB, wherein one or more mutations in an RB1 gene are detected in a sample obtained from the individual.

In other aspects, provided herein is an FGFR-targeted therapy for use in the manufacture of a medicament for treating or delaying progression of a UCB, wherein the medicament is to be administered to an individual having a UCB, wherein a deletion of an MTAP gene, or of a portion thereof, and one or more mutations in an FGFR3 gene have been detected in a sample obtained from the individual.

In other aspects, provided herein is a PTEN-targeted therapy for use in the manufacture of a medicament for treating or delaying progression of a UCB, wherein the medicament is to be administered to an individual having a UCB, wherein a deletion of an MTAP gene, or of a portion thereof, and one or more mutations in a PTEN gene have been detected in a sample obtained from the individual.

In other aspects, provided herein is an immunotherapy for use in the manufacture of a medicament for treating or delaying progression of a UCB, wherein the medicament is to be administered to an individual having a UCB, wherein a TMB of at least about 10 mut/Mb or at least about 20 mut/Mb, and/or a PD-L1-positive UCB have been detected in a sample obtained from the individual.

In other aspects, provided herein is an RB1-targeted therapy for use in the manufacture of a medicament for treating or delaying progression of a UCB, wherein the medicament is to be administered to an individual having a UCB, wherein one or more mutations in an RB1 gene have been detected in a sample obtained from the individual.

In other aspects, provided herein is an anti-cancer therapy for use in a method of treating or delaying progression of a UCB, wherein the method comprises administering an anti-cancer therapy to an individual having a UCB, wherein a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 are detected in a sample obtained from the individual.

In other aspects, provided herein is an anti-cancer therapy for use in a method of treating or delaying progression of a UCB, wherein the method comprises administering an anti-cancer therapy to an individual having a UCB, wherein one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 are detected in a sample obtained from the individual.

In other aspects, provided herein is an anti-cancer therapy for use in the manufacture of a medicament for treating or delaying progression of a UCB, wherein the medicament is to be administered to an individual having a UCB, wherein a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 have been detected in a sample obtained from the individual.

In other aspects, provided herein is an anti-cancer therapy for use in the manufacture of a medicament for treating or delaying progression of a UCB, wherein the medicament is to be administered to an individual having a UCB, wherein one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 have been detected in a sample obtained from the individual.

In some aspects, provided herein are methods of treating or delaying progression of a NSCLC. In other aspects, provided herein are methods of identifying one or more treatment options for an individual having a NSCLC. In other aspects, provided herein are methods of selecting treatment for an individual having a NSCLC. In other aspects, provided herein are methods of predicting survival of an individual having a NSCLC treated with an anti-cancer therapy. In other aspects, provided herein are methods of identifying an individual having a NSCLC who may benefit from a treatment comprising an anti-cancer therapy. In other aspects, provided herein are methods of selecting a therapy for an individual having a NSCLC.

In some embodiments, the NSCLC comprises a deletion of an MTAP gene, or of a portion thereof, and one or more mutations in an EGFR gene or in a SMARCA4 gene. In some embodiments, the NSCLC comprises a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1. In some embodiments, the methods comprise acquiring knowledge of a deletion of an MTAP gene, or of a portion thereof, and one or more mutations in an EGFR gene or in a SMARCA4 gene in a sample from an individual. In some embodiments, the methods comprise acquiring knowledge of a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1 in a sample from an individual. In some embodiments, the methods comprise detecting a deletion of an MTAP gene, or of a portion thereof, and one or more mutations in an EGFR gene or in a SMARCA4 gene in a sample from an individual. In some embodiments, the methods comprise detecting a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1 in a sample from an individual. In some embodiments, the methods comprise generating a report comprising one or more treatment options for the individual. In some embodiments, the methods comprise administering to an individual having a NSCLC an effective amount of a treatment comprising an anti-cancer therapy. In some embodiments, the NSCLC comprises a deletion of an MTAP gene, or of a portion thereof, and one or more mutations in an EGFR gene or in a SMARCA4 gene described herein. In some embodiments, the NSCLC comprises a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1. In some embodiments, the anti-cancer therapy is an anti-cancer therapy described herein. In some embodiments, the anti-cancer therapy is an EGFR-targeted therapy or a SMARCA4-targeted therapy. In some embodiments, the anti-cancer therapy is a PRMT5-targeted therapy. In some embodiments, the anti-cancer therapy is a combination therapy comprising an EGFR-targeted therapy or a SMARCA4-targeted therapy, and a PRMT5-targeted therapy.

In other aspects, provided herein are methods of detecting a deletion of an MTAP gene, or of a portion thereof, and one or more mutations in an EGFR gene or in a SMARCA4 gene. In other aspects, provided herein are methods of detecting a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1. In other aspects, provided herein are methods of assessing a deletion of an MTAP gene, or of a portion thereof, and one or more mutations in an EGFR gene or in a SMARCA4 gene. In other aspects, provided herein are methods of assessing a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1.

In other aspects, provided herein are methods of detecting the presence or absence of a NSCLC in an individual. In some embodiments, the methods comprise detecting the presence or absence of a NSCLC in a sample from an individual. In some embodiments, the methods comprise detecting the presence or absence of a deletion of an MTAP gene, or of a portion thereof, and one or more mutations in an EGFR gene in the sample; and/or detecting the presence or absence of a deletion of an MTAP gene, or of a portion thereof, and one or more mutations in a SMARCA4 gene in the sample.

In other aspects, provided herein are systems comprising one or more processors; and a non-transitory computer readable storage medium comprising one or more programs executable by the one or more processors for performing a method. In some embodiments, the method comprises: (a) sequencing one or more nucleic acids, thereby producing a plurality of sequencing reads, wherein the one or more nucleic acids are derived from a sample obtained from an individual having a NSCLC; (b) analyzing the plurality of sequencing reads for the presence of a deletion of an MTAP gene, or of a portion thereof, and one or more mutations in an EGFR gene or in a SMARCA4 gene; and (c) detecting, based on the analyzing step, a deletion of an MTAP gene, or of a portion thereof, and one or more mutations in an EGFR gene or in a SMARCA4 gene in the sample. In some embodiments, the method comprises: (a) sequencing one or more nucleic acids, thereby producing a plurality of sequencing reads, wherein the one or more nucleic acids are derived from a sample obtained from an individual having a NSCLC; (b) analyzing the plurality of sequencing reads for the presence of a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1; and (c) detecting, based on the analyzing step, a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1 in the sample. In some embodiments, a system provided herein comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a deletion of an MTAP gene, or of a portion thereof, and one or more mutations in an EGFR gene or in a SMARCA4 gene; and (c) detect, based on the analyzing, a deletion of an MTAP gene, or of a portion thereof, and one or more mutations in an EGFR gene or in a SMARCA4 gene in the sample. In some embodiments, a system provided herein comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1; and (c) detect, based on the analyzing, a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1 in the sample. In some embodiments, the sample is obtained from an individual having a NSCLC.

In some aspects, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a deletion of an MTAP gene, or of a portion thereof, and one or more mutations in an EGFR gene or in a SMARCA4 gene; and (c) detecting, using the one or more processors and based on the analyzing, a deletion of an MTAP gene, or of a portion thereof, and one or more mutations in an EGFR gene or in a SMARCA4 gene in the sample.

In some aspects, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1; and (c) detecting, using the one or more processors and based on the analyzing, a deletion of an MTAP gene, or of a portion thereof, and one or more mutations in one or more genes, wherein the one or more genes comprise CDKN2A, CDKN2B, TP53, KRAS, ALK, ROS1, NTRK1, STK11, KEAP1, PIK3CA, PTEN, MET, ERBB2, BRAF, or RB1 in the sample. In some embodiments, the sample is obtained from an individual having a NSCLC.

In some embodiments, the NSCLC comprises an intact MTAP gene. In some embodiments, the NSCLC comprises a tumor mutational burden (TMB) of at least about 10 mut/Mb or at least about 20 mut/Mb, a PD-L1-positive status, and/or one or more mutations in an RB1 gene, a KRAS gene, or a TP53 gene. In some embodiments, the one or more mutations in a KRAS gene result in a G12C amino acid substitution in a KRAS polypeptide encoded by the KRAS gene. In some embodiments, the NSCLC comprises a tumor mutational burden (TMB) of at least about 10 mut/Mb or at least about 20 mut/Mb, a PD-L1-positive status, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF. In some embodiments, the methods comprise acquiring knowledge of a TMB of at least about 10 mut/Mb or at least about 20 mut/Mb, a PD-L1-positive status, and/or one or more mutations in an RB1 gene, a KRAS gene (e.g., a mutation resulting in a G12C amino acid substitution in a KRAS polypeptide encoded by the KRAS gene), or a TP53 gene in a sample from an individual. In some embodiments, the methods comprise acquiring knowledge of a TMB of at least about 10 mut/Mb or at least about 20 mut/Mb, a PD-L1-positive status, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF in a sample from an individual. In some embodiments, the methods comprise detecting a TMB of at least about 10 mut/Mb or at least about 20 mut/Mb, a PD-L1-positive status, and/or one or more mutations in an RB1 gene, a KRAS gene (e.g., a mutation resulting in a G12C amino acid substitution in a KRAS polypeptide encoded by the KRAS gene), or a TP53 gene in a sample from an individual. In some embodiments, the methods comprise detecting a TMB of at least about 10 mut/Mb or at least about 20 mut/Mb, a PD-L1-positive status, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF in a sample from an individual. In some embodiments, the methods comprise generating a report comprising one or more treatment options for the individual. In some embodiments, the methods comprise administering to an individual having a NSCLC an effective amount of a treatment comprising an anti-cancer therapy. In some embodiments, the NSCLC comprises a TMB of at least about 10 mut/Mb or at least about 20 mut/Mb, a PD-L1-positive status, and/or one or more mutations in an RB1 gene, a KRAS gene (e.g., a mutation resulting in a G12C amino acid substitution in a KRAS polypeptide encoded by the KRAS gene), or a TP53 gene. In some embodiments, the NSCLC comprises a TMB of at least about 10 mut/Mb or at least about 20 mut/Mb, a PD-L1-positive status, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF. In some embodiments, the anti-cancer therapy is an anti-cancer therapy described herein. In some embodiments, the anti-cancer therapy is an immunotherapy, an RB1-targeted therapy, a KRAS-targeted therapy (e.g., a KRAS (G12C)-targeted therapy), or a TP53-targeted therapy.

In other aspects, provided herein are methods of detecting a TMB of at least about 10 mut/Mb or at least about 20 mut/Mb, a PD-L1-positive status, and/or one or more mutations in an RB1 gene, a KRAS gene (e.g., a mutation resulting in a G12C amino acid substitution in a KRAS polypeptide encoded by the KRAS gene), or a TP53 gene. In other aspects, provided herein are methods of detecting a TMB of at least about 10 mut/Mb or at least about 20 mut/Mb, a PD-L1-positive status, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF. In other aspects, provided herein are methods of assessing a TMB of at least about 10 mut/Mb or at least about 20 mut/Mb, a PD-L1-positive status, and/or one or more mutations in an RB1 gene, a KRAS gene (e.g., a mutation resulting in a G12C amino acid substitution in a KRAS polypeptide encoded by the KRAS gene), or a TP53 gene. In other aspects, provided herein are methods of assessing a TMB of at least about 10 mut/Mb or at least about 20 mut/Mb, a PD-L1-positive status, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF. In other aspects, provided herein are methods of detecting the presence or absence of a NSCLC in an individual. In some embodiments, the methods comprise detecting the presence or absence of a NSCLC in a sample from an individual. In some embodiments, the methods comprise detecting the presence or absence of a TMB of at least about 10 mut/Mb or at least about 20 mut/Mb, or a PD-L1-positive NSCLC in the sample. In some embodiments, the methods comprise detecting the presence or absence of one or more mutations in an RB1 gene in the sample. In some embodiments, the methods comprise detecting the presence or absence of one or more mutations in a KRAS gene (e.g., a mutation resulting in a G12C amino acid substitution in a KRAS polypeptide encoded by the KRAS gene) in the sample. In some embodiments, the methods comprise detecting the presence or absence of one or more mutations in a TP53 gene in the sample.

In other aspects, provided herein are systems comprising one or more processors; and a non-transitory computer readable storage medium comprising one or more programs executable by the one or more processors for performing a method. In some embodiments, the method comprises: (a) sequencing one or more nucleic acids, thereby producing a plurality of sequencing reads, wherein the one or more nucleic acids are derived from a sample obtained from an individual having a NSCLC; (b) analyzing the plurality of sequencing reads for the presence of a TMB of at least about 10 mut/Mb or at least about 20 mut/Mb, and/or one or more mutations in an RB1 gene, a KRAS gene (e.g., a mutation resulting in a G12C amino acid substitution in a KRAS polypeptide encoded by the KRAS gene), or a TP53 gene; and (c) detecting, based on the analyzing step, a TMB of at least about 10 mut/Mb or at least about 20 mut/Mb, and/or one or more mutations in an RB1 gene, a KRAS gene (e.g., a mutation resulting in a G12C amino acid substitution in a KRAS polypeptide encoded by the KRAS gene), or a TP53 gene in the sample. In some embodiments, the method comprises: (a) sequencing one or more nucleic acids, thereby producing a plurality of sequencing reads, wherein the one or more nucleic acids are derived from a sample obtained from an individual having a NSCLC; (b) analyzing the plurality of sequencing reads for the presence of a TMB of at least about 10 mut/Mb or at least about 20 mut/Mb, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF; and (c) detecting, based on the analyzing step, a TMB of at least about 10 mut/Mb or at least about 20 mut/Mb, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF in the sample. In some embodiments, a system provided herein comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a TMB of at least about 10 mut/Mb or at least about 20 mut/Mb, and/or one or more mutations in an RB1 gene, a KRAS gene (e.g., a mutation resulting in a G12C amino acid substitution in a KRAS polypeptide encoded by the KRAS gene), or a TP53 gene; and (c) detect, based on the analyzing, a TMB of at least about 10 mut/Mb or at least about 20 mut/Mb, and/or one or more mutations in an RB1 gene, a KRAS gene (e.g., a mutation resulting in a G12C amino acid substitution in a KRAS polypeptide encoded by the KRAS gene), or a TP53 gene in the sample. In some embodiments, a system provided herein comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a TMB of at least about 10 mut/Mb or at least about 20 mut/Mb, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF; and (c) detect, based on the analyzing, a TMB of at least about 10 mut/Mb or at least about 20 mut/Mb, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF in the sample. In some embodiments, the sample is obtained from an individual having a NSCLC.

In some aspects, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a TMB of at least about 10 mut/Mb or at least about 20 mut/Mb, and/or one or more mutations in an RB1 gene, a KRAS gene (e.g., a mutation resulting in a G12C amino acid substitution in a KRAS polypeptide encoded by the KRAS gene), or a TP53 gene; and (c) detecting, using the one or more processors and based on the analyzing, a TMB of at least about 10 mut/Mb or at least about 20 mut/Mb, and/or one or more mutations in an RB1 gene, a KRAS gene (e.g., a mutation resulting in a G12C amino acid substitution in a KRAS polypeptide encoded by the KRAS gene), or a TP53 gene in the sample.

In some aspects, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a TMB of at least about 10 mut/Mb or at least about 20 mut/Mb, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF; and (c) detecting, using the one or more processors and based on the analyzing, a TMB of at least about 10 mut/Mb or at least about 20 mut/Mb, and/or one or more mutations in one or more genes, wherein the one or more genes comprise EGFR, ALK, ROS1, NTRK1, STK11, KEAP1, PIK3CA, SMARCA4, PTEN, MET, ERBB2, or BRAF in the sample. In some embodiments, the sample is obtained from an individual having a NSCLC.

In some embodiments, the individual is a human.

In other aspects, provided herein is an EGFR-targeted therapy for use in a method of treating or delaying progression of a NSCLC, wherein the method comprises administering an EGFR-targeted therapy to an individual having a NSCLC, wherein a deletion of an MTAP gene, or of a portion thereof, and one or more mutations in an EGFR gene are detected in a sample obtained from the individual.

In other aspects, provided herein is a SMARCA4-targeted therapy for use in a method of treating or delaying progression of a NSCLC, wherein the method comprises administering a SMARCA4-targeted therapy to an individual having a NSCLC, wherein a deletion of an MTAP gene, or of a portion thereof, and one or more mutations in a SMARCA4 gene are detected in a sample obtained from the individual.

In other aspects, provided herein is an anti-cancer therapy for use in a method of treating or delaying progression of a NSCLC, wherein the method comprises administering an anti-cancer therapy to an individual having a NSCLC, wherein a deletion of an MTAP gene, or of a portion thereof, and one or more mutations in one or more genes, wherein the one or more genes comprise TP53, KRAS (e.g., a mutation resulting in a G12C amino acid substitution in a KRAS polypeptide encoded by the KRAS gene), ALK, ROS1, NTRK1, STK11, KEAP1, PIK3CA, PTEN, MET, ERBB2, BRAF, or RB1 are detected in a sample obtained from the individual.

In other aspects, provided herein is an immunotherapy for use in a method of treating or delaying progression of a NSCLC, wherein the method comprises administering an immunotherapy to an individual having a NSCLC, wherein a TMB of at least about 10 mut/Mb or at least about 20 mut/Mb, and/or a PD-L1-positive NSCLC are detected in a sample obtained from the individual. In some embodiments, the absence of a deletion of an MTAP gene, or of a portion thereof, is further detected in the sample.

In other aspects, provided herein is RB1-targeted therapy for use in a method of treating or delaying progression of a NSCLC, wherein the method comprises administering an RB1-targeted therapy to an individual having a NSCLC, wherein one or more mutations in an RB1 gene are detected in a sample obtained from the individual. In some embodiments, the absence of a deletion of an MTAP gene, or of a portion thereof, is further detected in the sample.

In other aspects, provided herein is a KRAS-targeted therapy for use in a method of treating or delaying progression of a NSCLC, wherein the method comprises administering a KRAS-targeted therapy to an individual having a NSCLC, wherein one or more mutations in a KRAS gene (e.g., a mutation resulting in a G12C amino acid substitution in a KRAS polypeptide encoded by the KRAS gene) are detected in a sample obtained from the individual. In some embodiments, the absence of a deletion of an MTAP gene, or of a portion thereof, is further detected in the sample. In some embodiments, the KRAS-targeted therapy is a KRAS (G12C)-targeted therapy.

In other aspects, provided herein is a TP53-targeted therapy for use in a method of treating or delaying progression of a NSCLC, wherein the method comprises administering a TP53-targeted therapy to an individual having a NSCLC, wherein one or more mutations in a TP53 gene are detected in a sample obtained from the individual. In some embodiments, the absence of a deletion of an MTAP gene, or of a portion thereof, is further detected in the sample.

In other aspects, provided herein is an anti-cancer therapy for use in a method of treating or delaying progression of a NSCLC, wherein the method comprises administering an anti-cancer therapy to an individual having a NSCLC, wherein one or more mutations in one or more genes, wherein the one or more genes comprise EGFR, ALK, ROS1, NTRK1, STK11, KEAP1, PIK3CA, SMARCA4, PTEN, MET, ERBB2, or BRAF, are detected in a sample obtained from the individual. In some embodiments, the absence of a deletion of an MTAP gene, or of a portion thereof, is further detected in the sample.

In other aspects, provided herein is an EGFR-targeted therapy for use in the manufacture of a medicament for treating or delaying progression of a NSCLC, wherein the medicament is to be administered to an individual having a NSCLC, wherein a deletion of an MTAP gene, or of a portion thereof, and one or more mutations in an EGFR gene are detected in a sample obtained from the individual.

In other aspects, provided herein is a SMARCA4-targeted therapy for use in the manufacture of a medicament for treating or delaying progression of a NSCLC, wherein the medicament is to be administered to an individual having a NSCLC, wherein a deletion of an MTAP gene, or of a portion thereof, and one or more mutations in a SMARCA4 gene are detected in a sample obtained from the individual.

In other aspects, provided herein is an immunotherapy for use in the manufacture of a medicament for treating or delaying progression of a NSCLC, wherein the medicament is to be administered to an individual having a NSCLC, wherein a TMB of at least about 10 mut/Mb or at least about 20 mut/Mb, and/or a PD-L1-positive NSCLC are detected in a sample obtained from the individual. In some embodiments, the absence of a deletion of an MTAP gene, or of a portion thereof, is further detected in the sample.

In other aspects, provided herein is an RB1-targeted therapy for use in the manufacture of a medicament for treating or delaying progression of a NSCLC, wherein the medicament is to be administered to an individual having a NSCLC, wherein one or more mutations in an RB1 gene are detected in a sample obtained from the individual. In some embodiments, the absence of a deletion of an MTAP gene, or of a portion thereof, is further detected in the sample.

In other aspects, provided herein is a KRAS-targeted therapy for use in the manufacture of a medicament for treating or delaying progression of a NSCLC, wherein the medicament is to be administered to an individual having a NSCLC, wherein one or more mutations in a KRAS gene (e.g., a mutation resulting in a G12C amino acid substitution in a KRAS polypeptide encoded by the KRAS gene) are detected in a sample obtained from the individual. In some embodiments, the absence of a deletion of an MTAP gene, or of a portion thereof, is further detected in the sample. In some embodiments, the KRAS-targeted therapy is a KRAS (G12C)-targeted therapy.

In other aspects, provided herein is a TP53-targeted therapy for use in the manufacture of a medicament for treating or delaying progression of a NSCLC, wherein the medicament is to be administered to an individual having a NSCLC, wherein one or more mutations in a TP53 gene are detected in a sample obtained from the individual. In some embodiments, the absence of a deletion of an MTAP gene, or of a portion thereof, is further detected in the sample.

In other aspects, provided herein is an anti-cancer therapy for use in the manufacture of a medicament for treating or delaying progression of a NSCLC, wherein the medicament is to be administered to an individual having a NSCLC, wherein one or more mutations in one or more genes, wherein the one or more genes comprise EGFR, ALK, ROS1, NTRK1, STK11, KEAP1, PIK3CA, SMARCA4, PTEN, MET, ERBB2, or BRAF, are detected in a sample obtained from the individual. In some embodiments, the absence of a deletion of an MTAP gene, or of a portion thereof, is further detected in the sample.

In other aspects, provided herein is an anti-cancer therapy for use in the manufacture of a medicament for treating or delaying progression of a NSCLC, wherein the medicament is to be administered to an individual having a NSCLC, wherein a deletion of an MTAP gene, or of a portion thereof, and one or more mutations in one or more genes, wherein the one or more genes comprise TP53, KRAS, ALK, ROS1, NTRK1, STK11, KEAP1, PIK3CA, PTEN, MET, ERBB2, BRAF, or RB1 are detected in a sample obtained from the individual.

In some aspects, provided herein are methods of treating or delaying progression of a srcRCC or a ccRCC. In other aspects, provided herein are methods of identifying one or more treatment options for an individual having a srcRCC or a ccRCC. In other aspects, provided herein are methods of selecting treatment for an individual having a srcRCC or a ccRCC. In other aspects, provided herein are methods of predicting survival of an individual having a srcRCC or a ccRCC treated with an anti-cancer therapy. In other aspects, provided herein are methods of identifying an individual having a srcRCC or a ccRCC who may benefit from a treatment comprising an anti-cancer therapy. In other aspects, provided herein are methods of selecting a therapy for an individual having a srcRCC or a ccRCC.

In some embodiments, the srcRCC or ccRCC comprises a deletion of an MTAP gene, or of a portion thereof, and one or more mutations in an NF2 gene. In some embodiments, the srcRCC or ccRCC comprises a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET. In some embodiments, the methods comprise acquiring knowledge of a deletion of an MTAP gene, or of a portion thereof, and one or more mutations in an NF2 gene in a sample from an individual. In some embodiments, the methods comprise acquiring knowledge of a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET in a sample from an individual. In some embodiments, the methods comprise detecting a deletion of an MTAP gene, or of a portion thereof, and one or more mutations in an NF2 gene in a sample from an individual. In some embodiments, the methods comprise detecting a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET in a sample from an individual. In some embodiments, the methods comprise generating a report comprising one or more treatment options for the individual. In some embodiments, the methods comprise administering to an individual having a srcRCC or ccRCC an effective amount of a treatment comprising an anti-cancer therapy. In some embodiments, the srcRCC or ccRCC comprises a deletion of an MTAP gene, or of a portion thereof, and one or more mutations in an NF2 gene described herein. In some embodiments, the srcRCC or ccRCC comprises a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET. In some embodiments, the anti-cancer therapy is an anti-cancer therapy described herein. In some embodiments, the anti-cancer therapy is an NF2-targeted therapy. In some embodiments, the anti-cancer therapy is a PRMT5-targeted therapy. In some embodiments, the anti-cancer therapy is a combination therapy comprising an NF2-targeted therapy and a PRMT5-targeted therapy.

In other aspects, provided herein are methods of detecting a deletion of an MTAP gene, or of a portion thereof, and one or more mutations in an NF2 gene. In other aspects, provided herein are methods of detecting a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET. In other aspects, provided herein are methods of assessing a deletion of an MTAP gene, or of a portion thereof, and one or more mutations in an NF2 gene. In other aspects, provided herein are methods of assessing a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET.

In other aspects, provided herein are methods of detecting the presence or absence of a srcRCC or a ccRCC in an individual. In some embodiments, the methods comprise detecting the presence or absence of a srcRCC or a ccRCC in a sample from an individual. In some embodiments, the methods comprise detecting the presence or absence of a deletion of an MTAP gene, or of a portion thereof, and one or more mutations in an NF2 gene in the sample.

In other aspects, provided herein are systems comprising one or more processors; and a non-transitory computer readable storage medium comprising one or more programs executable by the one or more processors for performing a method. In some embodiments, the method comprises: (a) sequencing one or more nucleic acids, thereby producing a plurality of sequencing reads, wherein the one or more nucleic acids are derived from a sample obtained from an individual having a srcRCC or ccRCC; (b) analyzing the plurality of sequencing reads for the presence of a deletion of an MTAP gene, or of a portion thereof, and one or more mutations in an NF2 gene; and (c) detecting, based on the analyzing step, a deletion of an MTAP gene, or of a portion thereof, and one or more mutations in an NF2 gene in the sample. In some embodiments, the method comprises: (a) sequencing one or more nucleic acids, thereby producing a plurality of sequencing reads, wherein the one or more nucleic acids are derived from a sample obtained from an individual having a srcRCC or ccRCC; (b) analyzing the plurality of sequencing reads for the presence of a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET; and (c) detecting, based on the analyzing step, a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET in the sample. In some embodiments, a system provided herein comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a deletion of an MTAP gene, or of a portion thereof, and one or more mutations in an NF2 gene; and (c) detect, based on the analyzing, a deletion of an MTAP gene, or of a portion thereof, and one or more mutations in an NF2 gene in the sample. In some embodiments, a system provided herein comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET; and (c) detect, based on the analyzing, a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET in the sample. In some embodiments, the sample is obtained from an individual having a srcRCC or ccRCC.

In some aspects, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a deletion of an MTAP gene, or of a portion thereof, and one or more mutations in an NF2 gene; and (c) detecting, using the one or more processors and based on the analyzing, a deletion of an MTAP gene, or of a portion thereof, and one or more mutations in an NF2 gene in the sample. In some embodiments, the sample is obtained from an individual having a srcRCC or ccRCC.

In some aspects, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET; and (c) detecting, using the one or more processors and based on the analyzing, a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET in the sample. In some embodiments, the sample is obtained from an individual having a srcRCC or ccRCC.

In some embodiments, the srcRCC or ccRCC comprises an intact MTAP gene. In some embodiments, the ccRCC comprises one or more mutations in a VHL gene or a PBRM1 gene. In some embodiments, the ccRCC comprises one or more mutations in one or more genes, wherein the one or more genes comprise SETD2, BAP1, TP53, PTEN, TERT, TSC1, PIK3CA, ATM, NF2, or MET. In some embodiments, the methods comprise acquiring knowledge of one or more mutations in a VHL gene or a PBRM1 gene in a sample from an individual. In some embodiments, the methods comprise acquiring knowledge of one or more mutations in one or more genes, wherein the one or more genes comprise SETD2, BAP1, TP53, PTEN, TERT, TSC1, PIK3CA, ATM, NF2, or MET in a sample from an individual. In some embodiments, the methods comprise detecting one or more mutations in a VHL gene or a PBRM1 gene in a sample from an individual. In some embodiments, the methods comprise detecting one or more mutations in one or more genes, wherein the one or more genes comprise SETD2, BAP1, TP53, PTEN, TERT, TSC1, PIK3CA, ATM, NF2, or MET in a sample from an individual. In some embodiments, the methods comprise generating a report comprising one or more treatment options for the individual. In some embodiments, the methods comprise administering to an individual having a ccRCC an effective amount of a treatment comprising an anti-cancer therapy. In some embodiments, the ccRCC comprises one or more mutations in a VHL gene or a PBRM1 gene. In some embodiments, the ccRCC comprises one or more mutations in one or more genes, wherein the one or more genes comprise SETD2, BAP1, TP53, PTEN, TERT, TSC1, PIK3CA, ATM, NF2, or MET. In some embodiments, the anti-cancer therapy is an anti-cancer therapy described herein. In some embodiments, the anti-cancer therapy is a VHL-targeted therapy, or a PBRM1-targeted therapy.

In other aspects, provided herein are methods of detecting one or more mutations in a VHL gene or a PBRM1 gene. In other aspects, provided herein are methods of detecting one or more mutations in one or more genes, wherein the one or more genes comprise SETD2, BAP1, TP53, PTEN, TERT, TSC1, PIK3CA, ATM, NF2, or MET. In other aspects, provided herein are methods of assessing one or more mutations in a VHL gene or a PBRM1 gene. In other aspects, provided herein are methods of assessing one or more mutations in one or more genes, wherein the one or more genes comprise SETD2, BAP1, TP53, PTEN, TERT, TSC1, PIK3CA, ATM, NF2, or MET.

In other aspects, provided herein are methods of detecting the presence or absence of a ccRCC in an individual. In some embodiments, the methods comprise detecting the presence or absence of a ccRCC in a sample from an individual. In some embodiments, the methods comprise detecting the presence or absence of one or more mutations in a VHL gene in the sample. In some embodiments, the methods comprise detecting the presence or absence of one or more mutations in a PBRM1 gene in the sample.

In other aspects, provided herein are systems comprising one or more processors; and a non-transitory computer readable storage medium comprising one or more programs executable by the one or more processors for performing a method. In some embodiments, the method comprises: (a) sequencing one or more nucleic acids, thereby producing a plurality of sequencing reads, wherein the one or more nucleic acids are derived from a sample obtained from an individual having a ccRCC; (b) analyzing the plurality of sequencing reads for the presence of one or more mutations in a VHL gene or a PBRM1 gene; and (c) detecting, based on the analyzing step, one or more mutations in a VHL gene or a PBRM1 gene in the sample. In some embodiments, the method comprises: (a) sequencing one or more nucleic acids, thereby producing a plurality of sequencing reads, wherein the one or more nucleic acids are derived from a sample obtained from an individual having a ccRCC; (b) analyzing the plurality of sequencing reads for the presence of one or more mutations in one or more genes, wherein the one or more genes comprise SETD2, BAP1, TP53, PTEN, TERT, TSC1, PIK3CA, ATM, NF2, or MET; and (c) detecting, based on the analyzing step, one or more mutations in one or more genes, wherein the one or more genes comprise SETD2, BAP1, TP53, PTEN, TERT, TSC1, PIK3CA, ATM, NF2, or MET in the sample. In some embodiments, a system provided herein comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of one or more mutations in a VHL gene or a PBRM1 gene; and (c) detect, based on the analyzing, one or more mutations in a VHL gene or a PBRM1 gene in the sample. In some embodiments, a system provided herein comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of one or more mutations in one or more genes, wherein the one or more genes comprise SETD2, BAP1, TP53, PTEN, TERT, TSC1, PIK3CA, ATM, NF2, or MET; and (c) detect, based on the analyzing, one or more mutations in one or more genes, wherein the one or more genes comprise SETD2, BAP1, TP53, PTEN, TERT, TSC1, PIK3CA, ATM, NF2, or MET in the sample. In some embodiments, the sample is obtained from an individual having a ccRCC.

In some aspects, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of one or more mutations in a VHL gene or a PBRM1 gene; and (c) detecting, using the one or more processors and based on the analyzing, one or more mutations in a VHL gene or a PBRM1 gene in the sample. In some embodiments, the sample is obtained from an individual having a ccRCC.

In some aspects, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of one or more mutations in one or more genes, wherein the one or more genes comprise SETD2, BAP1, TP53, PTEN, TERT, TSC1, PIK3CA, ATM, NF2, or MET; and (c) detecting, using the one or more processors and based on the analyzing, one or more mutations in one or more genes, wherein the one or more genes comprise SETD2, BAP1, TP53, PTEN, TERT, TSC1, PIK3CA, ATM, NF2, or MET in the sample. In some embodiments, the sample is obtained from an individual having a ccRCC.

In some embodiments, the individual is a human.

In other aspects, provided herein is an NF2-targeted therapy for use in a method of treating or delaying progression of a srcRCC or ccRCC, wherein the method comprises administering an NF2-targeted therapy to an individual having a srcRCC or ccRCC, wherein a deletion of the MTAP gene, or of the portion thereof, and one or more mutations in the NF2 gene are detected in a sample obtained from the individual.

In other aspects, provided herein is an anti-cancer therapy for use in a method of treating or delaying progression of a srcRCC or ccRCC, wherein the method comprises administering an anti-cancer therapy to an individual having a srcRCC or ccRCC, wherein a deletion of the MTAP gene, or of the portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET, are detected in a sample obtained from the individual.

In other aspects, provided herein is a VHL-targeted therapy for use in a method of treating or delaying progression of a ccRCC, wherein the method comprises administering a VHL-targeted therapy to an individual having a ccRCC, wherein one or more mutations in the VHL gene are detected in a sample obtained from the individual.

In other aspects, provided herein is a PBRM1-targeted therapy for use in a method of treating or delaying progression of a ccRCC, wherein the method comprises administering a PBRM1-targeted therapy to an individual having a ccRCC, wherein one or more mutations in the PBRM1 gene are detected in a sample obtained from the individual.

In other aspects, provided herein is an anti-cancer therapy for use in a method of treating or delaying progression of a cRCC, wherein the method comprises administering an anti-cancer therapy to an individual having a ccRCC, wherein one or more mutations in one or more genes, wherein the one or more genes comprise SETD2, BAP1, TP53, PTEN, TERT, TSC1, PIK3CA, ATM, NF2, or MET, are detected in a sample obtained from the individual.

In other aspects, provided herein is an NF2-targeted therapy for use in the manufacture of a medicament for treating or delaying progression of a srcRCC or ccRCC, wherein the medicament is to be administered to an individual having a srcRCC or ccRCC, wherein a deletion of the MTAP gene, or of the portion thereof, and one or more mutations in the NF2 gene are detected in a sample obtained from the individual.

In other aspects, provided herein is an anti-cancer therapy for use in the manufacture of a medicament for treating or delaying progression of a srcRCC or ccRCC, wherein the medicament is to be administered to an individual having a srcRCC or ccRCC, wherein a deletion of the MTAP gene, or of the portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, or MET, are detected in a sample obtained from the individual.

In other aspects, provided herein is a VHL-targeted therapy for use in the manufacture of a medicament for treating or delaying progression of a ccRCC, wherein the medicament is to be administered to an individual having a ccRCC, wherein one or more mutations in the VHL gene are detected in a sample obtained from the individual.

In other aspects, provided herein is a PBRM1-targeted therapy for use in the manufacture of a medicament for treating or delaying progression of a ccRCC, wherein the medicament is to be administered to an individual having a ccRCC, wherein one or more mutations in the PBRM1 gene are detected in a sample obtained from the individual.

In other aspects, provided herein is an anti-cancer therapy for use in the manufacture of a medicament for treating or delaying progression of a cRCC, wherein the medicament is to be administered to an individual having a ccRCC, wherein one or more mutations in one or more genes, wherein the one or more genes comprise SETD2, BAP1, TP53, PTEN, TERT, TSC1, PIK3CA, ATM, NF2, or MET, are detected in a sample obtained from the individual.

### Definitions

Before describing the invention in detail, it is to be understood that this invention is not limited to particular compositions or biological systems. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “or” is used herein to mean, and is used interchangeably with, the term “and/or”, unless context clearly indicates otherwise.

The terms “about” or “approximately” as used herein refer to the usual error range for the respective value readily known to the skilled person in this technical field, for example, an acceptable degree of error or deviation for the quantity measured given the nature or precision of the measurements. Reference to “about” or “approximately” a value or parameter herein includes (and describes) embodiments that are directed to that value or parameter per se.

The terms “fusion” or “fusion molecule” are used generically herein, and include any fusion molecule (e.g., a gene (e.g., in genomic DNA), a gene product (e.g., cDNA, mRNA, polypeptide, or protein), and variants thereof) that includes a fragment of a first gene or gene product and a fragment of a second gene or gene product described herein. A fusion molecule includes a “breakpoint” or “fusion junction,” which is the transition (i.e., direct fusion) point between the first gene or gene product, or fragment thereof, and the second gene or gene product, or fragment thereof.

The term “isolated” in the context of a nucleic acid molecule or a polypeptide refers to a nucleic acid molecule or polypeptide being separated from other nucleic acid molecules or polypeptides that are present in the natural source of the nucleic acid molecule or polypeptide. In some certain embodiments, the isolated nucleic acid molecule or polypeptide is free of or substantially free of other cellular material or culture medium when produced by recombinant techniques, or free of or substantially free of chemical precursors or other chemicals when chemically synthesized.

As used herein, the term “configured to hybridize to” indicates that a nucleic acid molecule has a nucleotide sequence with sufficient length and sequence complementarity to the nucleotide sequence of a target nucleic acid to allow the nucleic acid molecule to hybridize to the target nucleic acid, e.g., with a Tₘ of at least 65° C. in an aqueous solution of 1×SCC (150 mM sodium chloride and 15 mM trisodium citrate) and 0.1% SDS. Other hybridization conditions may be used when hybridizing a nucleic acid molecule to a target nucleic acid molecule, for example in the context of a described method.

“Percent (%) sequence identity” with respect to a reference polypeptide or polynucleotide sequence is defined as the percentage of amino acid residues or nucleotides in a sequence that are identical to the amino acid residues or nucleotides in the reference polypeptide or polynucleotide sequence, after aligning the sequences and introducing gaps, if necessary, to achieve the maximum percent sequence identity, and not considering any conservative substitutions as part of the sequence identity.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In some embodiments, the individual is human patient, e.g., a human patient having a cancer described herein.

An “effective amount” or a “therapeutically effective amount” of an agent, e.g., an anti-cancer agent, or a pharmaceutical formulation, refers to an amount effective, at dosages and for periods of time necessary, to achieve the desired therapeutic or prophylactic result, e.g., in the treatment or management of a cancer, for example, delaying or minimizing one or more symptoms associated with the cancer. In some embodiments, an effective amount or a therapeutically effective amount of an agent refers to an amount of the agent at dosages and for periods of time necessary, alone or in combination with other therapeutic agents, which provides a therapeutic or prophylactic benefit in the treatment or management of a disease such as a cancer. In some embodiments, an effective amount or a therapeutically effective amount of an agent enhances the therapeutic or prophylactic efficacy of another therapeutic agent or another therapeutic modality.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, delaying progression of disease, alleviation of symptoms, diminishment of any direct or indirect pathological consequences of the disease, decreasing the rate of disease progression, amelioration or palliation of the disease state, and remission or improved prognosis. In some embodiments, the terms “treatment,” “treat,” or “treating” include preventing a disease, such as cancer, e.g., before an individual begins to suffer from a cancer or from re-growth or recurrence of the cancer. In some embodiments, the terms “treatment,” “treat,” or “treating” include inhibiting or reducing the severity of a disease such as a cancer.

“Likely to” or “increased likelihood,” as used herein, refer to an increased probability that an event, item, object, thing or person will occur. Thus, in one example, an individual that is likely to respond to treatment with an anti-cancer therapy, e.g., an anti-cancer therapy provided herein, alone or in combination, has an increased probability of responding to treatment with the anti-cancer therapy alone or in combination, relative to a reference individual or group of individuals. “Unlikely to” refers to a decreased probability that an event, item, object, thing or person will occur relative to a reference individual or group of individuals. Thus, an individual that is unlikely to respond to treatment with an anti-cancer therapy, e.g., an anti-cancer therapy provided herein, alone or in combination, has a decreased probability of responding to treatment with the anti-cancer therapy, alone or in combination, relative to a reference individual or group of individuals.

“Sample,” as used herein, refers to a biological sample obtained or derived from a source of interest, as described herein.

### Methods of the Disclosure

In some aspects, provided herein are methods of treating or delaying progression of a urothelial bladder cancer (UCB), a non-small cell lung cancer (NSCLC), a sarcomatoid renal cell carcinoma (srcRCC), or a clear cell renal cell carcinoma (ccRCC) in an individual. In some embodiments, the UCB, NSCLC, srcRCC, or ccRCC comprises a deletion of an methylthioadenosine phosphorylase (MTAP) gene, or of a portion thereof (i.e., MTAP-deleted UCB, NSCLC, srcRCC, or ccRCC). In some embodiments, the UCB, NSCLC, srcRCC, or ccRCC does not comprise a deletion of an MTAP gene (i.e., MTAP-intact UCB, NSCLC, srcRCC, or ccRCC).

In other aspects, methods are provided for identifying one or more treatment options for an individual having a UCB, NSCLC, srcRCC, or ccRCC, such as an MTAP-deleted UCB, NSCLC, srcRCC, or ccRCC; or an MTAP-intact UCB, NSCLC, srcRCC, or ccRCC. In other aspects, methods are provided for selecting treatment for an individual having a UCB, NSCLC, srcRCC, or ccRCC, such as an MTAP-deleted UCB, NSCLC, srcRCC, or ccRCC; or an MTAP-intact UCB, NSCLC, srcRCC, or ccRCC. In some aspects, methods are provided for predicting survival of an individual having a UCB, NSCLC, srcRCC, or ccRCC, such as an MTAP-deleted UCB, NSCLC, srcRCC, or ccRCC; or an MTAP-intact UCB, NSCLC, srcRCC, or ccRCC, treated with an anti-cancer therapy. In other aspects, provided herein are methods of identifying an individual having a UCB, NSCLC, srcRCC, or ccRCC, such as an MTAP-deleted UCB, NSCLC, srcRCC, or ccRCC; or an MTAP-intact UCB, NSCLC, srcRCC, or ccRCC, who may benefit from a treatment comprising an anti-cancer therapy. In other aspects, provided herein are methods of selecting a therapy for an individual having a UCB, NSCLC, srcRCC, or ccRCC, such as an MTAP-deleted UCB, NSCLC, srcRCC, or ccRCC; or an MTAP-intact UCB, NSCLC, srcRCC, or ccRCC. In other aspects, provided herein are methods of assessing, screening, or diagnosing an individual having a UCB, NSCLC, srcRCC, or ccRCC, such as an MTAP-deleted UCB, NSCLC, srcRCC, or ccRCC; or an MTAP-intact UCB, NSCLC, srcRCC, or ccRCC.

In some embodiments, the methods comprise acquiring knowledge of or detecting in a sample from an individual having a UCB, e.g., an MTAP-deleted UCB, a deletion of an MTAP gene, or of a portion thereof, and one or more mutations in an FGFR3 gene or in a PTEN gene. In some embodiments, the methods comprise acquiring knowledge of or detecting in a sample from an individual having a UCB, e.g., an MTAP-deleted UCB, a deletion of an MTAP gene, or of a portion thereof, and one or more mutations in one or more genes, wherein the one or more genes comprise CDKN2A, CDKN2B, TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1.

In some embodiments, the methods comprise acquiring knowledge of or detecting in a sample from an individual having a UCB, e.g., an MTAP-intact UCB, a tumor mutational burden (TMB) of at least about 10 mut/Mb or at least about 20 mut/Mb, a PD-L1-positive UCB, or one or more mutations in an RB1 gene. In some embodiments, the methods comprise acquiring knowledge of or detecting in a sample from an individual having a UCB, e.g., an MTAP-intact UCB, a tumor mutational burden (TMB) of at least about 10 mut/Mb or at least about 20 mut/Mb, a PD-L1-positive UCB, or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2.

In some embodiments, the methods comprise acquiring knowledge of or detecting in a sample from an individual having a NSCLC, e.g., an MTAP-deleted NSCLC, a deletion of an MTAP gene, or of a portion thereof, and one or more mutations in an EGFR gene or in a SMARCA4 gene. In some embodiments, the methods comprise acquiring knowledge of or detecting in a sample from an individual having a NSCLC, e.g., an MTAP-deleted NSCLC, a deletion of an MTAP gene, or of a portion thereof, and one or more mutations in one or more genes, wherein the one or more genes comprise CDKN2A, CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1.

In some embodiments, the methods comprise acquiring knowledge of or detecting in a sample from an individual having a NSCLC, e.g., an MTAP-intact NSCLC, a tumor mutational burden (TMB) of at least about 10 mut/Mb or at least about 20 mut/Mb, a PD-L1-positive NSCLC, or one or more mutations in an RB1 gene, a KRAS gene (e.g., a mutation resulting in a G12C amino acid substitution in a KRAS polypeptide encoded by the KRAS gene), or a TP53 gene. In some embodiments, the methods comprise acquiring knowledge of or detecting in a sample from an individual having a NSCLC, e.g., an MTAP-intact NSCLC, a tumor mutational burden (TMB) of at least about 10 mut/Mb or at least about 20 mut/Mb, a PD-L1-positive NSCLC, or one or more mutations in one or more genes, wherein the one or more genes comprise CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1.

In some embodiments, the methods comprise acquiring knowledge of or detecting in a sample from an individual having a srcRCC or a ccRCC, e.g., an MTAP-deleted srcRCC or a ccRCC, a deletion of an MTAP gene, or of a portion thereof, and one or more mutations in an NF2 gene. In some embodiments, the methods comprise acquiring knowledge of or detecting in a sample from an individual having a srcRCC or a ccRCC, e.g., an MTAP-deleted srcRCC or a ccRCC, a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, NF2, or MET.

In some embodiments, the methods comprise acquiring knowledge of or detecting in a sample from an individual having a ccRCC, e.g., an MTAP-intact ccRCC, one or more mutations in a VHL gene or a PBRM1 gene. In some embodiments, the methods comprise acquiring knowledge of or detecting in a sample from an individual having a ccRCC, e.g., an MTAP-intact ccRCC, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, NF2, or MET.

Urothelial Bladder Cancer

Urothelial bladder cancer (UCB), also known as transitional cell carcinoma of the bladder, affects the cells from the transitional epithelium lining the inner surface of the bladder. Several variants of UCB exist, including plasmacytoid, nested, micropapillary, lipoid cell, sarcomatoid, microcystic, lymphoepithelioma-like, inverted papilloma-like, or clear cell UCB. Localized UCB can often present as more than one tumor on the bladder with low malignant potential. UCB can also present as non-invasive form and the more aggressive muscle-invasive form, depending on whether it is found only in the bladder mucosa and connective tissue, or whether it invades the smooth muscle layer in the bladder, respectively. In some instances, aggressive UCB can progress to metastatic UCB, with common sites of metastasis including lymph nodes, bone, lung, liver and peritoneoum.

In some embodiments, a UCB of the disclosure is a non-invasive UCB. In other embodiments, a UCB of the disclosure is an invasive UCB. In some embodiments, a UCB of the disclosure is a chemorefractory UCB. In some embodiments, a UCB of the disclosure is a metastatic UCB. In some embodiments, the metastatic UCB has metastasized to the lymph node, liver, lung, bone and/or peritoneum.

In some embodiments, a UCB of the disclosure contains a deletion in the MTAP gene (e.g., a deletion of the entire MTAP gene, or of a portion of the MTAP gene). The MTAP gene encodes a methyl-thioadenosine phosphorylase that plays a major role in the metabolism of polyamines such as adenosine and methionine. In some embodiments, the deletion in the MTAP gene is a deletion of a portion of thereof. In some embodiments, the deletion in the MTAP gene is a deletion of the entire MTAP gene. In some embodiments, the deletion in the MTAP gene is a heterozygous deletion. In other embodiments, the deletion in the MTAP gene is a homozygous deletion. In some embodiments, the deletion is a deletion at the 9p21 locus. In some embodiments, a UCB of the disclosure contains a deletion of a CDKN2A gene or a CDKN2B gene. In some embodiments, the deletion of the CDKN2A gene or the CDKN2B gene is a deletion of a portion of thereof. In some embodiments, the deletion of the CDKN2A gene or the CDKN2B gene is a deletion of the entire CDKN2A gene or CDKN2B gene. In some embodiments, the deletion of the CDKN2A gene or the CDKN2B gene is a heterozygous deletion. In other embodiments, the deletion of the CDKN2A gene or the CDKN2B gene is a homozygous deletion. In some embodiments, the deletion is a deletion at the 9p21 locus. In some embodiments, a UCB of the disclosure contains a co-deletion of an MTAP gene, or of a portion thereof, and of a CDKN2A gene and/or a CDKN2B gene, or portions thereof, e.g., at the 9p21 locus.

In some embodiments, a UCB of the disclosure contains a deletion in the MTAP gene (e.g., a deletion of the entire MTAP gene, or of a portion of the MTAP gene) and one or more mutations in one or more genes, wherein the one or more genes comprise CDKN2A, CDKN2B, TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, a UCB of the disclosure contains a deletion in the MTAP gene (e.g., a deletion of the entire MTAP gene, or of a portion of the MTAP gene) and one or more mutations in the FGFR3 gene. In some embodiments, the UCB contains a deletion in the MTAP gene (e.g., a deletion of the entire MTAP gene, or of a portion of the MTAP gene) and one or more mutations in the PTEN gene.

In some embodiments, a UCB of the disclosure contains an intact MTAP gene (e.g., lacks a deletion of an MTAP gene, or of a portion thereof). In some embodiments, a UCB of the disclosure contains an intact MTAP gene and is PD-L1-positive. In some embodiments, a UCB of the disclosure contains an intact MTAP gene and has a tumor mutational burden (TMB) of at least about 10 mut/Mb or at least about 20 mut/Mb. In some embodiments, a UCB of the disclosure contains an intact MTAP gene and one or more mutations in one or more genes, wherein the one or more genes comprise TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, a UCB of the disclosure contains an intact MTAP gene and one or more mutations in the RB1 gene.

### MTAP-Deleted Urothelial Bladder Cancer

In some aspects, provided herein are methods of treating or delaying progression of a urothelial bladder cancer (UCB) in an individual, wherein the UCB comprises a deletion of an methylthioadenosine phosphorylase (MTAP) gene, or of a portion thereof (MTAP-deleted UCB).

In other aspects, methods are provided for identifying one or more treatment options for an individual having an MTAP-deleted UCB. In other aspects, methods are provided for selecting treatment for an individual having an MTAP-deleted UCB. In some aspects, methods are provided for predicting survival of an individual having an MTAP-deleted UCB treated with an anti-cancer therapy. In other aspects, provided herein are methods of identifying an individual having an MTAP-deleted UCB, who may benefit from a treatment comprising an anti-cancer therapy. In other aspects, provided herein are methods of selecting a therapy for an individual having an MTAP-deleted UCB. In other aspects, provided herein are methods of assessing, screening, or diagnosing an individual having an MTAP-deleted UCB.

In some embodiments, the methods comprise acquiring knowledge of or detecting in a sample from an individual having an MTAP-deleted UCB a deletion of an MTAP gene, or of a portion thereof, and one or more mutations in an FGFR3 gene or in a PTEN gene. In some embodiments, the methods comprise acquiring knowledge of or detecting in a sample from an individual having an MTAP-deleted UCB a deletion of an MTAP gene, or of a portion thereof, and one or more mutations in one or more genes, wherein the one or more genes comprise CDKN2A, CDKN2B, TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1.

Methods of Selecting or Identifying a Treatment

In some aspects, provided herein are methods of identifying an individual having cancer, e.g., a UCB, who may benefit from an anti-cancer therapy, e.g., an anti-cancer therapy provided herein. In some embodiments, the methods comprise detecting a deletion of an MTAP gene, or of a portion thereof, and one or more mutations in an FGFR3 gene or in a PTEN gene in a sample obtained from the individual. In some embodiments, the methods comprise acquiring knowledge of the presence of a deletion of an MTAP gene, or of a portion thereof, and one or more mutations in an FGFR3 gene or in a PTEN gene in a sample obtained from the individual. In some embodiments, the presence of the deletion of an MTAP gene, or of a portion thereof, and one or more mutations in an FGFR3 gene or in a PTEN gene in the sample identifies the individual as one who may benefit from a treatment comprising an anti-cancer therapy, e.g., an anti-cancer therapy provided herein, such as an FGFR-targeted therapy or a PTEN-targeted therapy. In some embodiments, detection of the deletion of an MTAP gene, or of a portion thereof, and one or more mutations in an FGFR3 gene or in a PTEN gene in the sample identifies the individual as one who may benefit from a treatment comprising an anti-cancer therapy, e.g., an anti-cancer therapy provided herein, such as an FGFR-targeted therapy or a PTEN-targeted therapy. In some embodiments, responsive to knowledge of the deletion of an MTAP gene, or of a portion thereof, and one or more mutations in an FGFR3 gene or in a PTEN gene in the sample, the individual is identified as one who may benefit from a treatment comprising an anti-cancer therapy, e.g., an anti-cancer therapy provided herein, such as an FGFR-targeted therapy or a PTEN-targeted therapy. In some embodiments, the presence of the deletion of an MTAP gene, or of a portion thereof, and one or more mutations in an FGFR3 gene in the sample identifies the individual as one who may benefit from a treatment comprising an anti-cancer therapy, e.g., an anti-cancer therapy provided herein, such as an FGFR-targeted therapy. In some embodiments, detection of the deletion of an MTAP gene, or of a portion thereof, and one or more mutations in an FGFR3 gene in the sample identifies the individual as one who may benefit from a treatment comprising an anti-cancer therapy, e.g., an anti-cancer therapy provided herein, such as an FGFR-targeted therapy. In some embodiments, responsive to knowledge of the deletion of an MTAP gene, or of a portion thereof, and one or more mutations in an FGFR3 gene in the sample, the individual is identified as one who may benefit from a treatment comprising an anti-cancer therapy, e.g., an anti-cancer therapy provided herein, such as an FGFR-targeted therapy. In some embodiments, the presence of the deletion of an MTAP gene, or of a portion thereof, and one or more mutations in a PTEN gene in the sample identifies the individual as one who may benefit from a treatment comprising an anti-cancer therapy, e.g., an anti-cancer therapy provided herein, such as a PTEN-targeted therapy. In some embodiments, detection of the deletion of an MTAP gene, or of a portion thereof, and one or more mutations in a PTEN gene in the sample identifies the individual as one who may benefit from a treatment comprising an anti-cancer therapy, e.g., an anti-cancer therapy provided herein, such as a PTEN-targeted therapy. In some embodiments, responsive to knowledge of the deletion of an MTAP gene, or of a portion thereof, and one or more mutations in a PTEN gene in the sample, the individual is identified as one who may benefit from a treatment comprising an anti-cancer therapy, e.g., an anti-cancer therapy provided herein, such as a PTEN-targeted therapy. In some embodiments, the deletion of an MTAP gene, or of a portion thereof, and one or more mutations in an FGFR3 gene or in a PTEN gene are detected using any suitable method known in the art or described herein. In some embodiments, the sample is a sample described herein. In some embodiments, the sample comprises cells from the cancer or is obtained from cells from the cancer, e.g., a UCB. In some embodiments, the presence of the deletion of an MTAP gene, or of a portion thereof, and one or more mutations in an FGFR3 gene or in a PTEN gene in the sample identifies the individual as one who may benefit from a treatment comprising a PRMT5-targeted therapy and an additional cancer therapy, e.g., an FGFR-targeted therapy or a PTEN-targeted therapy. In some embodiments, detection of the deletion of an MTAP gene, or of a portion thereof, and one or more mutations in an FGFR3 gene or in a PTEN gene in the sample identifies the individual as one who may benefit from a treatment comprising a PRMT5-targeted therapy and an additional cancer therapy, e.g., an FGFR-targeted therapy or a PTEN-targeted therapy. In some embodiments, responsive to knowledge of the deletion of an MTAP gene, or of a portion thereof, and one or more mutations in an FGFR3 gene or in a PTEN gene in the sample, the individual is identified as one who may benefit from a treatment comprising a PRMT5-targeted therapy and an additional cancer therapy, e.g., an FGFR-targeted therapy or a PTEN-targeted therapy.

Also provided herein are methods of identifying or selecting a treatment, a therapy, or one or more treatment options for an individual having a cancer, such as a cancer described herein. In some embodiments, the cancer comprises a deletion of an MTAP gene, or of a portion thereof, and one or more mutations in an FGFR3 gene or in a PTEN gene. In some embodiments, the methods comprise detecting the deletion of an MTAP gene, or of a portion thereof, and one or more mutations in an FGFR3 gene or in a PTEN gene in a sample obtained from an individual having a cancer, such as a cancer described herein, e.g., a UCB. In some embodiments, the deletion of an MTAP gene, or of a portion thereof, and one or more mutations in an FGFR3 gene or in a PTEN gene are detected using any suitable method known in the art or described herein. In some embodiments, detection of a deletion of an MTAP gene, or of a portion thereof, and one or more mutations in an FGFR3 gene or in a PTEN gene in a sample obtained from an individual having a cancer, e.g., a cancer described herein, e.g., a UCB, identifies the individual as one who may benefit from an anti-cancer therapy, e.g., an anti-cancer therapy provided herein, such as an FGFR-targeted therapy or a PTEN-targeted therapy. In some embodiments, the presence of a deletion of an MTAP gene, or of a portion thereof, and one or more mutations in an FGFR3 gene or in a PTEN gene in a sample obtained from an individual having a cancer, e.g., a cancer provided herein, e.g., a UCB, identifies the individual as one who may benefit from an anti-cancer therapy, such as an anti-cancer therapy provided herein, e.g., an FGFR-targeted therapy or a PTEN-targeted therapy. In some embodiments, responsive to detection of a deletion of an MTAP gene, or of a portion thereof, and one or more mutations in an FGFR3 gene or in a PTEN gene in a sample obtained from an individual having a cancer, e.g., a cancer provided herein, e.g., a UCB, the individual is classified as a candidate to receive an anti-cancer therapy, such as an anti-cancer therapy provided herein, e.g., an FGFR-targeted therapy or a PTEN-targeted therapy. In some embodiments, responsive to detection of a deletion of an MTAP gene, or of a portion thereof, and one or more mutations in an FGFR3 gene or in a PTEN gene in a sample obtained from an individual having a cancer, e.g., a cancer provided herein, e.g., a UCB, the individual is classified or identified as likely to respond to an anti-cancer therapy, such as an anti-cancer therapy provided herein, e.g., an FGFR-targeted therapy or a PTEN-targeted therapy. In some embodiments, the sample is a sample described herein. In some embodiments, detection of a deletion of an MTAP gene, or of a portion thereof, and one or more mutations in an FGFR3 gene in a sample obtained from an individual having a cancer, e.g., a cancer described herein, e.g., a UCB, identifies the individual as one who may benefit from an anti-cancer therapy, e.g., an anti-cancer therapy provided herein, such as an FGFR-targeted therapy. In some embodiments, the presence of a deletion of an MTAP gene, or of a portion thereof, and one or more mutations in an FGFR3 gene in a sample obtained from an individual having a cancer, e.g., a cancer provided herein, e.g., a UCB, identifies the individual as one who may benefit from an anti-cancer therapy, such as an anti-cancer therapy provided herein, e.g., an FGFR-targeted therapy. In some embodiments, responsive to detection of a deletion of an MTAP gene, or of a portion thereof, and one or more mutations in an FGFR3 gene in a sample obtained from an individual having a cancer, e.g., a cancer provided herein, e.g., a UCB, the individual is classified as a candidate to receive an anti-cancer therapy, such as an anti-cancer therapy provided herein, e.g., an FGFR-targeted therapy. In some embodiments, responsive to detection of a deletion of an MTAP gene, or of a portion thereof, and one or more mutations in an FGFR3 gene in a sample obtained from an individual having a cancer, e.g., a cancer provided herein, e.g., a UCB, the individual is classified or identified as likely to respond to an anti-cancer therapy, such as an anti-cancer therapy provided herein, e.g., an FGFR-targeted therapy. In some embodiments, detection of a deletion of an MTAP gene, or of a portion thereof, and one or more mutations in a PTEN gene in a sample obtained from an individual having a cancer, e.g., a cancer described herein, e.g., a UCB, identifies the individual as one who may benefit from an anti-cancer therapy, e.g., an anti-cancer therapy provided herein, such as a PTEN-targeted therapy. In some embodiments, the presence of a deletion of an MTAP gene, or of a portion thereof, and one or more mutations in a PTEN gene in a sample obtained from an individual having a cancer, e.g., a cancer provided herein, e.g., a UCB, identifies the individual as one who may benefit from an anti-cancer therapy, such as an anti-cancer therapy provided herein, e.g., a PTEN-targeted therapy. In some embodiments, responsive to detection of a deletion of an MTAP gene, or of a portion thereof, and one or more mutations in a PTEN gene in a sample obtained from an individual having a cancer, e.g., a cancer provided herein, e.g., a UCB, the individual is classified as a candidate to receive an anti-cancer therapy, such as an anti-cancer therapy provided herein, e.g., a PTEN-targeted therapy. In some embodiments, responsive to detection of a deletion of an MTAP gene, or of a portion thereof, and one or more mutations in a PTEN gene in a sample obtained from an individual having a cancer, e.g., a cancer provided herein, e.g., a UCB, the individual is classified or identified as likely to respond to an anti-cancer therapy, such as an anti-cancer therapy provided herein, e.g., a PTEN-targeted therapy. In some embodiments, the sample is a sample described herein. In some embodiments, the sample comprises cells from the cancer or is obtained from cells from the cancer, e.g., a UCB. In some embodiments, the methods further comprise generating a report, e.g., as described herein. In some embodiments, the report comprises a treatment, a therapy, or one or more treatment options identified or selected for the individual, e.g., based at least in part on detection of a deletion of an MTAP gene, or of a portion thereof, and one or more mutations in an FGFR3 gene or in a PTEN gene in the sample. In some embodiments, detection of a deletion of an MTAP gene, or of a portion thereof, and one or more mutations in an FGFR3 gene or in a PTEN gene in a sample obtained from an individual having a cancer, e.g., a cancer described herein, e.g., a UCB, identifies the individual as one who may benefit from a PRMT5-targeted therapy and an additional anti-cancer therapy, e.g., an FGFR-targeted therapy or a PTEN-targeted therapy. In some embodiments, the presence of a deletion of an MTAP gene, or of a portion thereof, and one or more mutations in an FGFR3 gene or in a PTEN gene in a sample obtained from an individual having a cancer, e.g., a cancer provided herein, e.g., a UCB, identifies the individual as one who may benefit from a PRMT5-targeted therapy and an additional anti-cancer therapy, e.g., an FGFR-targeted therapy or a PTEN-targeted therapy. In some embodiments, responsive to detection of a deletion of an MTAP gene, or of a portion thereof, and one or more mutations in an FGFR3 gene or in a PTEN gene in a sample obtained from an individual having a cancer, e.g., a cancer provided herein, e.g., a UCB, the individual is classified as a candidate to receive a PRMT5-targeted therapy and an additional anti-cancer therapy, e.g., an FGFR-targeted therapy or a PTEN-targeted therapy. In some embodiments, responsive to detection of a deletion of an MTAP gene, or of a portion thereof, and one or more mutations in an FGFR3 gene or in a PTEN gene in a sample obtained from an individual having a cancer, e.g., a cancer provided herein, e.g., a UCB, the individual is classified or identified as likely to respond to a PRMT5-targeted therapy and an additional anti-cancer therapy, e.g., an FGFR-targeted therapy or a PTEN-targeted therapy.

In some aspects, provided herein are methods of identifying an individual having cancer, e.g., a UCB, who may benefit from an anti-cancer therapy, e.g., an anti-cancer therapy provided herein. In some embodiments, the methods comprise detecting a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual. In some embodiments, the methods comprise acquiring knowledge of the presence of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual. In some embodiments, the presence of the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample identifies the individual as one who may benefit from a treatment comprising an anti-cancer therapy, e.g., an anti-cancer therapy provided herein. In some embodiments, detection of the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample identifies the individual as one who may benefit from a treatment comprising an anti-cancer therapy, e.g., an anti-cancer therapy provided herein. In some embodiments, responsive to knowledge of the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample, the individual is identified as one who may benefit from a treatment comprising an anti-cancer therapy, e.g., an anti-cancer therapy provided herein. In some embodiments, the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 are detected using any suitable method known in the art or described herein. In some embodiments, the sample is a sample described herein. In some embodiments, the sample comprises cells from the cancer or is obtained from cells from the cancer, e.g., a UCB. In some embodiments, the presence of the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample identifies the individual as one who may benefit from a treatment comprising a PRMT5-targeted therapy and an additional cancer therapy, e.g., an anti-cancer therapy provided herein. In some embodiments, detection of the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample identifies the individual as one who may benefit from a treatment comprising a PRMT5-targeted therapy and an additional cancer therapy, e.g., an anti-cancer therapy provided herein. In some embodiments, responsive to knowledge of the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample, the individual is identified as one who may benefit from a treatment comprising a PRMT5-targeted therapy and an additional cancer therapy, e.g., an anti-cancer therapy provided herein.

Also provided herein are methods of identifying or selecting a treatment, a therapy, or one or more treatment options for an individual having a cancer, such as a cancer described herein. In some embodiments, the cancer comprises a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the methods comprise detecting the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual having a cancer, such as a cancer described herein, e.g., a UCB. In some embodiments, the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 are detected using any suitable method known in the art or described herein. In some embodiments, detection of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual having a cancer, e.g., a cancer described herein, e.g., a UCB, identifies the individual as one who may benefit from an anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual having a cancer, e.g., a cancer provided herein, e.g., a UCB, identifies the individual as one who may benefit from an anti-cancer therapy, such as an anti-cancer therapy provided herein. In some embodiments, responsive to detection of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual having a cancer, e.g., a cancer provided herein, e.g., a UCB, the individual is classified as a candidate to receive an anti-cancer therapy, such as an anti-cancer therapy provided herein. In some embodiments, responsive to detection of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual having a cancer, e.g., a cancer provided herein, e.g., a UCB, the individual is classified or identified as likely to respond to an anti-cancer therapy, such as an anti-cancer therapy provided herein. In some embodiments, the sample is a sample described herein. In some embodiments, the sample comprises cells from the cancer or is obtained from cells from the cancer, e.g., a UCB. In some embodiments, the methods further comprise generating a report, e.g., as described herein. In some embodiments, the report comprises a treatment, a therapy, or one or more treatment options identified or selected for the individual, e.g., based at least in part on detection of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, detection of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual having a cancer, e.g., a cancer described herein, e.g., a UCB, identifies the individual as one who may benefit from a PRMT5-targeted therapy and an additional anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual having a cancer, e.g., a cancer provided herein, e.g., a UCB, identifies the individual as one who may benefit from a PRMT5-targeted therapy and an additional anti-cancer therapy, e.g., an anti-cancer therapy provided herein. In some embodiments, responsive to detection of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual having a cancer, e.g., a cancer provided herein, e.g., a UCB, the individual is classified as a candidate to receive a PRMT5-targeted therapy and an additional anti-cancer therapy, e.g., an anti-cancer therapy provided herein. In some embodiments, responsive to detection of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual having a cancer, e.g., a cancer provided herein, e.g., a UCB, the individual is classified or identified as likely to respond to a PRMT5-targeted therapy and an additional anti-cancer therapy, e.g., an anti-cancer therapy provided herein.

In some embodiments of any of the methods provided herein, the methods further comprise acquiring knowledge of or detecting in a sample from an individual a deletion of a CDKN2A gene, or of a portion thereof; and/or a deletion of a CDKN2B gene, or of a portion thereof. In some embodiments of any of the methods provided herein, the individual has a deletion of a CDKN2A gene, or of a portion thereof; and/or a deletion of a CDKN2B gene, or of a portion thereof. In some embodiments of any of the methods provided herein, the individual has a heterozygous or a homozygous deletion of a CDKN2A gene, or of a portion thereof; and/or a heterozygous or a homozygous deletion of a CDKN2B gene, or of a portion thereof. In some embodiments of any of the methods provided herein, the individual has a heterozygous or a homozygous deletion of the MTAP gene, or of a portion thereof.

### Methods of Diagnosing, Assessing, Screening, Monitoring or Predicting

In some aspects, provided herein are methods of diagnosing or assessing a deletion of an MTAP gene, or of a portion thereof, and one or more mutations in one or more genes selected from an FGFR3 gene or a PTEN gene in a sample from an individual having a cancer, such as a UCB. In some embodiments, the methods comprise acquiring knowledge of the presence of a deletion of an MTAP gene, or of a portion thereof, and one or more mutations in an FGFR3 gene or in a PTEN gene in a sample obtained from the individual. In some embodiments, the methods comprise detecting a deletion of an MTAP gene, or of a portion thereof, and one or more mutations in an FGFR3 gene or in a PTEN gene in a sample obtained from the individual. In some embodiments, a deletion of an MTAP gene, or of a portion thereof, and one or more mutations in an FGFR3 gene or in a PTEN gene are detected in a sample obtained from the individual using any method known in the art, such as one or more of the methods of detection of nucleic acid molecules or polypeptides described herein. In some embodiments, the methods further comprise providing a diagnosis or an assessment of the deletion of the MTAP gene, or of the portion thereof, and one or more mutations in an FGFR3 gene or in a PTEN gene. In some embodiments, the diagnosis or assessment identifies the presence or absence of a deletion of an MTAP gene, or of a portion thereof, and one or more mutations in an FGFR3 gene or in a PTEN gene in the sample. In some embodiments, the diagnosis or assessment identifies the cancer, such as a UCB provided herein, as likely to respond to an anti-cancer therapy, e.g., an anti-cancer therapy provided herein, such as an FGFR-targeted therapy and/or a PTEN-targeted therapy. In some embodiments, the presence of a deletion of an MTAP gene, or of a portion thereof, and one or more mutations in an FGFR3 gene or in a PTEN gene in the sample identifies the cancer as likely to respond to an anti-cancer therapy, e.g., an anti-cancer therapy provided herein, such as an FGFR-targeted therapy or a PTEN-targeted therapy. In some embodiments, the diagnosis or assessment identifies the presence or absence of a deletion of an MTAP gene, or of a portion thereof, and one or more mutations in an FGFR3 gene in the sample. In some embodiments, the diagnosis or assessment identifies the cancer, such as a UCB provided herein, as likely to respond to an anti-cancer therapy, e.g., an anti-cancer therapy provided herein, such as an FGFR-targeted therapy. In some embodiments, the presence of a deletion of an MTAP gene, or of a portion thereof, and one or more mutations in an FGFR3 gene in the sample identifies the cancer as likely to respond to an anti-cancer therapy, e.g., an anti-cancer therapy provided herein, such as an FGFR-targeted therapy. In some embodiments, the diagnosis or assessment identifies the presence or absence of a deletion of an MTAP gene, or of a portion thereof, and one or more mutations in a PTEN gene in the sample. In some embodiments, the diagnosis or assessment identifies the cancer, such as a UCB provided herein, as likely to respond to an anti-cancer therapy, e.g., an anti-cancer therapy provided herein, such as a PTEN-targeted therapy. In some embodiments, the presence of a deletion of an MTAP gene, or of a portion thereof, and one or more mutations in a PTEN gene in the sample identifies the cancer as likely to respond to an anti-cancer therapy, e.g., an anti-cancer therapy provided herein, such as a PTEN-targeted therapy. In some embodiments, the sample is a sample described herein. In some embodiments, the sample comprises cells from the cancer or is obtained from cells from the cancer, e.g., from a UCB. In some embodiments, the individual has a cancer, is suspected of having a cancer, is being tested for a cancer, is being treated for a cancer, or is being tested for a susceptibility to a cancer, e.g., a UCB. In some embodiments, the diagnosis or assessment identifies the cancer, such as a UCB provided herein, as likely to respond to a PRMT5-targeted therapy and an additional anti-cancer therapy, e.g., an FGFR-targeted therapy or a PTEN-targeted therapy. In some embodiments, the presence of a deletion of an MTAP gene, or of a portion thereof, and one or more mutations in an FGFR gene or in a PTEN gene in the sample identifies the cancer as likely to respond to a PRMT5-targeted therapy and an additional anti-cancer therapy, e.g., an FGFR-targeted therapy and/or a PTEN-targeted therapy.

In some aspects, provided herein are methods of diagnosing or assessing a cancer in an individual, e.g., a UCB. In some embodiments, the methods of diagnosing or assessing cancer comprise detecting a deletion of an MTAP gene, or of a portion thereof, and one or more mutations in an FGFR3 gene or in a PTEN gene in a sample obtained from the individual, e.g., a sample comprising cells from the cancer, e.g., a UCB. In some embodiments, the methods comprise detecting a deletion of an MTAP gene, or of a portion thereof, and one or more mutations in an FGFR3 gene or in a PTEN gene in a sample obtained from the individual using any method known in the art, such as one or more of the methods of detection of nucleic acid molecules or polypeptides described herein. In some embodiments, detection of a deletion of an MTAP gene, or of a portion thereof, and one or more mutations in an FGFR3 gene or in a PTEN gene in a sample obtained from the individual identifies the cancer, e.g., the UCB, as likely to respond to an anti-cancer therapy, e.g., an anti-cancer therapy provided herein, such as an FGFR-targeted therapy or a PTEN-targeted therapy. In some embodiments, the presence of a deletion of an MTAP gene, or of a portion thereof, and one or more mutations in an FGFR3 gene or in a PTEN gene in a sample obtained from the individual identifies the cancer, e.g., the UCB, as likely to respond to an anti-cancer therapy, e.g., an anti-cancer therapy provided herein, such as an FGFR-targeted therapy or a PTEN-targeted therapy. In some embodiments, the methods further comprise providing a diagnosis or an assessment of the cancer, e.g., the UCB, or of the deletion of the MTAP gene, or of a portion thereof, and one or more mutations in an FGFR3 gene or in a PTEN gene. In some embodiments, the diagnosis or assessment identifies the cancer as likely to respond to an anti-cancer therapy, e.g., an anti-cancer therapy provided herein, such as an FGFR-targeted therapy or a PTEN-targeted therapy. In some embodiments, detection of a deletion of an MTAP gene, or of a portion thereof, and one or more mutations in an FGFR3 gene in a sample obtained from the individual identifies the cancer, e.g., the UCB, as likely to respond to an anti-cancer therapy, e.g., an anti-cancer therapy provided herein, such as an FGFR-targeted therapy. In some embodiments, the presence of a deletion of an MTAP gene, or of a portion thereof, and one or more mutations in an FGFR3 gene in a sample obtained from the individual identifies the cancer, e.g., the UCB, as likely to respond to an anti-cancer therapy, e.g., an anti-cancer therapy provided herein, such as an FGFR-targeted therapy. In some embodiments, the methods further comprise providing a diagnosis or an assessment of the cancer, e.g., the UCB, or of the deletion of the MTAP gene, or of a portion thereof, and one or more mutations in an FGFR3 gene. In some embodiments, the diagnosis or assessment identifies the cancer as likely to respond to an anti-cancer therapy, e.g., an anti-cancer therapy provided herein, such as an FGFR-targeted therapy. In some embodiments, detection of a deletion of an MTAP gene, or of a portion thereof, and one or more mutations in a PTEN gene in a sample obtained from the individual identifies the cancer, e.g., the UCB, as likely to respond to an anti-cancer therapy, e.g., an anti-cancer therapy provided herein, such as a PTEN-targeted therapy. In some embodiments, the presence of a deletion of an MTAP gene, or of a portion thereof, and one or more mutations in a PTEN gene in a sample obtained from the individual identifies the cancer, e.g., the UCB, as likely to respond to an anti-cancer therapy, e.g., an anti-cancer therapy provided herein, such as a PTEN-targeted therapy. In some embodiments, the methods further comprise providing a diagnosis or an assessment of the cancer, e.g., the UCB, or of the deletion of the MTAP gene, or of a portion thereof, and one or more mutations in a PTEN gene. In some embodiments, the diagnosis or assessment identifies the cancer as likely to respond to an anti-cancer therapy, e.g., an anti-cancer therapy provided herein, such as a PTEN-targeted therapy. In some embodiments, the diagnosis or assessment identifies the presence or absence of the deletion of the MTAP gene, or of a portion thereof, and one or more mutations in an FGFR3 gene or in a PTEN gene in the sample. In some embodiments, detection of a deletion of an MTAP gene, or of a portion thereof, and one or more mutations in an FGFR3 gene or in a PTEN gene in a sample obtained from the individual identifies the cancer, e.g., the UCB, as likely to respond to a PRMT5-targeted therapy and an additional anti-cancer therapy, e.g., an FGFR-targeted therapy or a PTEN-targeted therapy. In some embodiments, the presence of a deletion of an MTAP gene, or of a portion thereof, and one or more mutations in an FGFR3 gene or in a PTEN gene in a sample obtained from the individual identifies the cancer, e.g., the UCB, as likely to respond to a PRMT5-targeted therapy and an additional anti-cancer therapy, e.g., an FGFR-targeted therapy or a PTEN-targeted therapy. In some embodiments, the diagnosis or assessment identifies the cancer as likely to respond to a PRMT5-targeted therapy and an additional anti-cancer therapy, e.g., an FGFR-targeted therapy or a PTEN-targeted therapy.

In some aspects, provided herein are methods of predicting survival of an individual having a cancer, e.g., a cancer provided herein, e.g., a UCB. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in an FGFR3 gene or in a PTEN gene in a sample from the individual. In some embodiments, the methods comprise detecting a deletion of an MTAP gene, or of a portion thereof, and one or more mutations in an FGFR3 gene or in a PTEN gene in a sample from the individual. In some embodiments, responsive to acquiring knowledge of a deletion of an MTAP gene, or of a portion thereof, and one or more mutations in an FGFR3 gene or in a PTEN gene in the sample, the individual is predicted to have longer survival after treatment with an anti-cancer therapy, e.g., an anti-cancer therapy provided herein (e.g., an FGFR-targeted therapy or a PTEN-targeted therapy), for example, as compared to an individual whose cancer does not exhibit the deletion of the MTAP gene, or of a portion thereof, and one or more mutations in an FGFR3 gene or in a PTEN gene. In some embodiments, responsive to detecting a deletion of an MTAP gene, or of a portion thereof, and one or more mutations in an FGFR3 gene or in a PTEN gene in the sample, the individual is predicted to have longer survival after treatment with an anti-cancer therapy, e.g., an anti-cancer therapy provided herein (e.g., an FGFR-targeted therapy or a PTEN-targeted therapy), for example, as compared to an individual whose cancer does not exhibit the deletion of the MTAP gene, or of a portion thereof, and one or more mutations in an FGFR3 gene or in a PTEN gene. In some embodiments, responsive to acquiring knowledge of a deletion of an MTAP gene, or of a portion thereof, and one or more mutations in an FGFR3 gene in the sample, the individual is predicted to have longer survival after treatment with an anti-cancer therapy, e.g., an anti-cancer therapy provided herein (e.g., an FGFR-targeted therapy), for example, as compared to an individual whose cancer does not exhibit the deletion of the MTAP gene, or of a portion thereof, and one or more mutations in an FGFR3 gene. In some embodiments, responsive to detecting a deletion of an MTAP gene, or of a portion thereof, and one or more mutations in an FGFR3 gene in the sample, the individual is predicted to have longer survival after treatment with an anti-cancer therapy, e.g., an anti-cancer therapy provided herein (e.g., an FGFR-targeted therapy), for example, as compared to an individual whose cancer does not exhibit the deletion of the MTAP gene, or of a portion thereof, and one or more mutations in an FGFR3 gene. In some embodiments, responsive to acquiring knowledge of a deletion of an MTAP gene, or of a portion thereof, and one or more mutations in a PTEN gene in the sample, the individual is predicted to have longer survival after treatment with an anti-cancer therapy, e.g., an anti-cancer therapy provided herein (e.g., a PTEN-targeted therapy), for example, as compared to an individual whose cancer does not exhibit the deletion of the MTAP gene, or of a portion thereof, and one or more mutations in a PTEN gene In some embodiments, responsive to detecting a deletion of an MTAP gene, or of a portion thereof, and one or more mutations in a PTEN gene in the sample, the individual is predicted to have longer survival after treatment with an anti-cancer therapy, e.g., an anti-cancer therapy provided herein (e.g., a PTEN-targeted therapy), for example, as compared to an individual whose cancer does not exhibit the deletion of the MTAP gene, or of a portion thereof, and one or more mutations in a PTEN gene. In some embodiments, the methods further comprise providing a diagnosis or an assessment. In some embodiments, the diagnosis or assessment identifies the presence or absence of the deletion of an MTAP gene, or of a portion thereof, and one or more mutations in an FGFR3 gene or in a PTEN gene in the sample. In some embodiments, the diagnosis or assessment identifies the individual as being predicted to have longer survival after treatment with an anti-cancer therapy, e.g., an anti-cancer therapy provided herein (e.g., an FGFR-targeted therapy or a PTEN-targeted therapy), for example, as compared to an individual whose cancer does not exhibit the deletion of the MTAP gene, or of a portion thereof, and one or more mutations in an FGFR3 gene or in a PTEN gene. In some embodiments, the diagnosis or assessment identifies the individual as being predicted to have longer survival after treatment with an anti-cancer therapy, e.g., an anti-cancer therapy provided herein (e.g., an FGFR-targeted therapy), for example, as compared to an individual whose cancer does not exhibit the deletion of the MTAP gene, or of a portion thereof, and one or more mutations in an FGFR3 gene. In some embodiments, the diagnosis or assessment identifies the individual as being predicted to have longer survival after treatment with an anti-cancer therapy, e.g., an anti-cancer therapy provided herein (e.g., a PTEN-targeted therapy), for example, as compared to an individual whose cancer does not exhibit the deletion of the MTAP gene, or of a portion thereof, and one or more mutations in a PTEN gene. In some embodiments, the sample is a sample described herein. In some embodiments, the sample comprises cells from the cancer or is obtained from cells from the cancer, e.g., the UCB. In some embodiments, responsive to acquiring knowledge of a deletion of an MTAP gene, or of a portion thereof, and one or more mutations in an FGFR3 gene or in a PTEN gene in the sample, the individual is predicted to have longer survival after treatment with a PRMT5-targeted therapy and an additional anti-cancer therapy, e.g., an FGFR-targeted therapy or a PTEN-targeted therapy, for example, as compared to an individual whose cancer does not exhibit the deletion of the MTAP gene, or of a portion thereof, and one or more mutations in an FGFR3 gene or in a PTEN gene. In some embodiments, responsive to detecting a deletion of an MTAP gene, or of a portion thereof, and one or more mutations in an FGFR3 gene or in a PTEN gene in the sample, the individual is predicted to have longer survival after treatment with a PRMT5-targeted therapy and an additional anti-cancer therapy, e.g., an FGFR-targeted therapy or a PTEN-targeted therapy, for example, as compared to an individual whose cancer does not exhibit the deletion of the MTAP gene, or of a portion thereof, and one or more mutations in an FGFR3 gene or in a PTEN gene. In some embodiments, the diagnosis or assessment identifies the individual as being predicted to have longer survival after treatment with a PRMT5-targeted therapy and an additional anti-cancer therapy, e.g., an FGFR-targeted therapy or a PTEN-targeted therapy, for example, as compared to an individual whose cancer does not exhibit the deletion of the MTAP gene, or of a portion thereof, and one or more mutations in an FGFR3 gene or in a PTEN gene.

In some aspects, provided herein are methods of screening an individual having cancer, suspected of having cancer, being tested for cancer, being treated for cancer, or being tested for a susceptibility to cancer, e.g., a UCB. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in an FGFR3 gene or in a PTEN gene in a sample from the individual. In some embodiments, the methods comprise detecting a deletion of an MTAP gene, or of a portion thereof, and one or more mutations in an FGFR3 gene or in a PTEN gene in a sample from the individual. In some embodiments, responsive to acquiring knowledge of a deletion of an MTAP gene, or of a portion thereof, and one or more mutations in an FGFR3 gene or in a PTEN gene in the sample, the individual is predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in an FGFR3 gene or in a PTEN gene. In some embodiments, responsive to detecting a deletion of an MTAP gene, or of a portion thereof, and one or more mutations in an FGFR3 gene or in a PTEN gene in the sample, the individual is predicted to have increased risk of cancer recurrence, aggressive cancer, anti-cancer therapy resistance, or poor prognosis, for example, as compared to an individual whose cancer does not exhibit the deletion of an MTAP gene, or of a portion thereof, and one or more mutations in an FGFR3 gene or in a PTEN gene. In some embodiments, the methods further comprise providing a diagnosis or an assessment. In some embodiments, the diagnosis or assessment identifies the presence or absence of the deletion of the MTAP gene, or of a portion thereof, and one or more mutations in an FGFR3 gene or in a PTEN gene in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in an FGFR3 gene or in a PTEN gene. In some embodiments, the sample is a sample described herein. In some embodiments, the sample comprises cells from the cancer or is obtained from cells from the cancer, e.g., the UCB.

In some embodiments, the methods further comprise selectively enriching for one or more nucleic acids comprising nucleotide sequences that comprise one or more mutations in an FGFR3 gene or in a PTEN gene to produce an enriched sample, e.g., using a reagent known in the art or provided herein, such as a bait, probe, or oligonucleotide described herein. In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample. In some embodiments, the methods further comprise selectively enriching for one or more polypeptides comprising amino acid sequences that comprise one or more mutations in an FGFR3 gene or in a PTEN gene to produce an enriched sample, e.g., using a reagent known in the art or provided herein, such as an antibody described herein. In some embodiments, the methods further comprise selectively enriching for one or more nucleic acids comprising nucleotide sequences that comprise a deletion of an MTAP gene, or of a portion thereof, to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise a deletion of an MTAP gene, or of a portion thereof, to produce an enriched sample, e.g., using a reagent known in the art or provided herein, such as an antibody described herein.

In some aspects, provided herein are methods of diagnosing or assessing a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample from an individual having a cancer, such as a UCB. In some embodiments, the methods comprise acquiring knowledge of the presence of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual. In some embodiments, the methods comprise detecting a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual. In some embodiments, a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 are detected in a sample obtained from the individual using any method known in the art, such as one or more of the methods of detection of nucleic acid molecules or polypeptides described herein. In some embodiments, the methods further comprise providing a diagnosis or an assessment of the deletion of the MTAP gene, or of the portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the diagnosis or assessment identifies the presence or absence of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample. In some embodiments, the diagnosis or assessment identifies the cancer, such as a UCB provided herein, as likely to respond to an anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample identifies the cancer as likely to respond to an anti-cancer therapy, e.g., an anti-cancer therapy provided herein. In some embodiments, the sample is a sample described herein. In some embodiments, the sample comprises cells from the cancer or is obtained from cells from the cancer, e.g., from a UCB. In some embodiments, the individual has a cancer, is suspected of having a cancer, is being tested for a cancer, is being treated for a cancer, or is being tested for a susceptibility to a cancer, e.g., a UCB. In some embodiments, the diagnosis or assessment identifies the cancer, such as a UCB provided herein, as likely to respond to a PRMT5-targeted therapy and an additional anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample identifies the cancer as likely to respond to a PRMT5-targeted therapy and an additional anti-cancer therapy, e.g., an anti-cancer therapy provided herein.

In some aspects, provided herein are methods of diagnosing or assessing a cancer in an individual, e.g., a UCB. In some embodiments, the methods of diagnosing or assessing cancer comprise detecting a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual, e.g., a sample comprising cells from the cancer, e.g., a UCB. In some embodiments, the methods comprise detecting a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual using any method known in the art, such as one or more of the methods of detection of nucleic acid molecules or polypeptides described herein. In some embodiments, detection of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual identifies the cancer, e.g., the UCB, as likely to respond to an anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual identifies the cancer, e.g., the UCB, as likely to respond to an anti-cancer therapy, e.g., an anti-cancer therapy provided herein. In some embodiments, the methods further comprise providing a diagnosis or an assessment of the cancer, e.g., the UCB, or of the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the diagnosis or assessment identifies the cancer as likely to respond to an anti-cancer therapy, e.g., an anti-cancer therapy provided herein. In some embodiments, the diagnosis or assessment identifies the presence or absence of the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1.

In some embodiments, detection of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual identifies the cancer, e.g., the UCB, as likely to respond to a PRMT5-targeted therapy and an additional anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual identifies the cancer, e.g., the UCB, as likely to respond to a PRMT5-targeted therapy and an additional anti-cancer therapy, e.g., an anti-cancer therapy provided herein. In some embodiments, the diagnosis or assessment identifies the cancer as likely to respond to a PRMT5-targeted therapy and an additional anti-cancer therapy, e.g., an anti-cancer therapy provided herein.

In some aspects, provided herein are methods of predicting survival of an individual having a cancer, e.g., a cancer provided herein, e.g., a UCB. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, orBAPI in a sample from the individual. In some embodiments, the methods comprise detecting a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample from the individual. In some embodiments, responsive to acquiring knowledge of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample, the individual is predicted to have longer survival after treatment with an anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, responsive to detecting a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample, the individual is predicted to have longer survival after treatment with an anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the methods further comprise providing a diagnosis or an assessment. In some embodiments, the diagnosis or assessment identifies the presence or absence of the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the sample is a sample described herein. In some embodiments, the sample comprises cells from the cancer or is obtained from cells from the cancer, e.g., the UCB. In some embodiments, responsive to acquiring knowledge of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample, the individual is predicted to have longer survival after treatment with a PRMT5-targeted therapy and an additional anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, responsive to detecting a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample, the individual is predicted to have longer survival after treatment with a PRMT5-targeted therapy and an additional anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the diagnosis or assessment identifies the individual as being predicted to have longer survival after treatment with a PRMT5-targeted therapy and an additional anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1.

In some aspects, provided herein are methods of screening an individual having cancer, suspected of having cancer, being tested for cancer, being treated for cancer, or being tested for a susceptibility to cancer, e.g., a UCB. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample from the individual. In some embodiments, the methods comprise detecting a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample from the individual. In some embodiments, responsive to acquiring knowledge of a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample, the individual is predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, responsive to detecting a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample, the individual is predicted to have increased risk of cancer recurrence, aggressive cancer, anti-cancer therapy resistance, or poor prognosis, for example, as compared to an individual whose cancer does not exhibit the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the methods further comprise providing a diagnosis or an assessment. In some embodiments, the diagnosis or assessment identifies the presence or absence of the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the sample is a sample described herein. In some embodiments, the sample comprises cells from the cancer or is obtained from cells from the cancer, e.g., the UCB.

In some embodiments, the methods further comprise selectively enriching for one or more nucleic acids comprising nucleotide sequences that comprise one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 to produce an enriched sample, e.g., using a reagent known in the art or provided herein, such as a bait, probe, or oligonucleotide described herein. In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample. In some embodiments, the methods further comprise selectively enriching for one or more polypeptides comprising amino acid sequences that comprise one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 to produce an enriched sample, e.g., using a reagent known in the art or provided herein, such as an antibody described herein. In some embodiments, the methods further comprise selectively enriching for one or more nucleic acids comprising nucleotide sequences that comprise a deletion of an MTAP gene, or of a portion thereof, to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise a deletion of an MTAP gene, or of a portion thereof, to produce an enriched sample, e.g., using a reagent known in the art or provided herein, such as an antibody described herein.

In some embodiments of any of the methods provided herein, the methods further comprise acquiring knowledge of or detecting in a sample from an individual a deletion of a CDKN2A gene, or of a portion thereof; and/or a deletion of a CDKN2B gene, or of a portion thereof. In some embodiments of any of the methods provided herein, the individual has a deletion of a CDKN2A gene, or of a portion thereof; and/or a deletion of a CDKN2B gene, or of a portion thereof. In some embodiments of any of the methods provided herein, the individual has a heterozygous or a homozygous deletion of a CDKN2A gene, or of a portion thereof; and/or a heterozygous or a homozygous deletion of a CDKN2B gene, or of a portion thereof. In some embodiments of any of the methods provided herein, the individual has a heterozygous or a homozygous deletion of the MTAP gene, or of a portion thereof.

Methods of Treatment

Also provided herein are methods of treating or delaying progression of a cancer in an individual, such as a cancer provided herein, e.g., a UCB. In some embodiments, the individual has a cancer comprising a deletion of an MTAP gene, or of a portion thereof, and one or more mutations in an FGFR3 gene or in a PTEN gene. In some embodiments, the methods of treating or delaying progression of a cancer of the disclosure, e.g., a UCB comprising a deletion of an MTAP gene, or of a portion thereof, and one or more mutations in an FGFR3 gene or in a PTEN gene, comprise administering to the individual a therapeutically effective amount of an anti-cancer therapy, such as an anti-cancer therapy provided herein, e.g., an FGFR-targeted therapy or a PTEN-targeted therapy. In some embodiments, the methods of treating or delaying progression of a cancer of the disclosure, e.g., a UCB comprising a deletion of an MTAP gene, or of a portion thereof, and one or more mutations in an FGFR3 gene or in a PTEN gene, comprise administering to the individual an effective amount of an anti-cancer therapy, such as an anti-cancer therapy provided herein (e.g., an FGFR-targeted therapy or a PTEN-targeted therapy), responsive to knowledge of the presence of the deletion of an MTAP gene, or of a portion thereof, and one or more mutations in an FGFR3 gene or in a PTEN gene in a sample obtained from the individual. In some embodiments, the sample is a sample described herein. In some embodiments, the sample comprises cells from the cancer or is obtained from cells from the cancer. The methods of treatment disclosed herein may include any of the anti-cancer therapies and/or therapeutic agents described herein, e.g., infra.

In some embodiments, the individual has a cancer comprising a deletion of an MTAP gene, or of a portion thereof, and one or more mutations in an FGFR3 gene. In some embodiments, the methods of treating or delaying progression of a cancer of the disclosure, e.g., a UCB comprising a deletion of an MTAP gene, or of a portion thereof, and one or more mutations in an FGFR3 gene, comprise administering to the individual a therapeutically effective amount of an anti-cancer therapy, such as an anti-cancer therapy provided herein, e.g., an FGFR-targeted therapy. In some embodiments, the methods of treating or delaying progression of a cancer of the disclosure, e.g., a UCB comprising a deletion of an MTAP gene, or of a portion thereof, and one or more mutations in an FGFR3 gene, comprise administering to the individual an effective amount of an anti-cancer therapy, such as an anti-cancer therapy provided herein (e.g., an FGFR-targeted therapy), responsive to knowledge of the presence of the deletion of an MTAP gene, or of a portion thereof, and one or more mutations in an FGFR3 gene in a sample obtained from the individual. In some embodiments, the FGFR-targeted therapy comprises one or more of a multi-kinase inhibitor, an FGFR-selective inhibitor, an FGFR3-specific inhibitor, or a combination therapy. In some embodiments, the multi-kinase inhibitor comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), or orantinib (TSU-68). In some embodiments, the FGFR-selective inhibitor comprises one or more of PRN1371, XL-228, AZ12908010 (AZ8010), Debio-1347 (CH5183284), FIIN-2, LY2874455, Infigratinib (BGJ398, NVP-BGJ398), Pemigatinib (Pemazyre®, INCB054828), Erdafitinib (JNJ-42756493, Balversa®), ASP5878, TAS-120, PRN1371, or PKC412. In some embodiments, the FGFR3-specific inhibitor comprises one or more of Vofatamab (B-701, anti-FGFR3 antibody), or MFGR1877S (anti-FGFR3 antibody). In some embodiments, the combination therapy comprises one or more of: an FGFR-targeted therapy (e.g., an FGFR-targeted therapy provided herein) administered in combination with a PD-1/PD-L1-targeted therapy (e.g., a PD-1- or PD-L1-targeted therapy provided herein); an FGFR-targeted therapy (e.g., an FGFR-targeted therapy provided herein) administered in combination with an EGFR inhibitor (e.g., an EGFR inhibitor provided herein); an FGFR-targeted therapy (e.g., an FGFR-targeted therapy provided herein) administered in combination with an immunotherapy (e.g., an immunotherapy provided herein); an FGFR-targeted therapy (e.g., an FGFR-targeted therapy provided herein) administered in combination with a MAPK inhibitor (e.g., a MPAK inhibitor provided herein); an FGFR-targeted therapy (e.g., an FGFR-targeted therapy provided herein) administered in combination with a PI3K inhibitor (e.g., a PI3K inhibitor provided herein); an FGFR-targeted therapy (e.g., an FGFR-targeted therapy provided herein) administered in combination with an AKT inhibitor (e.g., an AKT inhibitor provided herein); or an FGFR-targeted therapy (e.g., an FGFR-targeted therapy provided herein) administered in combination with a VEGF inhibitor (e.g., a VEGF inhibitor provided herein). In some embodiments, the anti-cancer therapy is administered in combination with a second anti-cancer therapy or agent, such as any of the anti-cancer agents or therapies described herein. In some embodiments, the second anti-cancer therapy or agent comprises a PRMT5-targeted therapy. In some embodiments, the anti-cancer therapy comprises an FGFR-targeted therapy and a PRMT5-targeted therapy. In some embodiments, the PRMT5-targeted therapy comprises one or more of DW14800, PJ-68, EPZ004777, YQ36286, CMP5, PR5-LL-CM01, III EPZ015666 (GSK3235025), LLY-283, GSK3326595 (EPZ015938), JNJ-64619178, PF-06939999, CTx-034, GSK591, HLCL65, GSK3203591, or DS-437.

In some embodiments, the individual has a cancer comprising a deletion of an MTAP gene, or of a portion thereof, and one or more mutations in a PTEN gene. In some embodiments, the methods of treating or delaying progression of a cancer of the disclosure, e.g., a UCB comprising a deletion of an MTAP gene, or of a portion thereof, and one or more mutations in a PTEN gene, comprise administering to the individual a therapeutically effective amount of an anti-cancer therapy, such as an anti-cancer therapy provided herein, e.g., a PTEN-targeted therapy. In some embodiments, the methods of treating or delaying progression of a cancer of the disclosure, e.g., a UCB comprising a deletion of an MTAP gene, or of a portion thereof, and one or more mutations in a PTEN gene, comprise administering to the individual an effective amount of an anti-cancer therapy, such as an anti-cancer therapy provided herein (e.g., a PTEN-targeted therapy), responsive to knowledge of the presence of the deletion of an MTAP gene, or of a portion thereof, and one or more mutations in a PTEN gene in a sample obtained from the individual. In some embodiments, the PTEN-targeted therapy comprises one or more of a PI3K inhibitor (e.g., a PI3K inhibitor provided herein), an AKT inhibitor (e.g., an AKT inhibitor provided herein), an mTOR inhibitor (e.g., an mTOR inhibitor provided herein), or a MET inhibitor (e.g., a MET inhibitor provided herein). In some embodiments, the PTEN-targeted therapy is administered in combination with an additional anti-cancer therapy, such as a hormone therapy agent (e.g., a hormone therapy agent described herein), a chemotherapeutic agent (e.g., a chemotherapeutic agent provided herein), an mTOR inhibitor (e.g., an mTOR inhibitor provided herein), or an EGFR inhibitor (e.g., an EGFR inhibitor provided herein). In some embodiments, the anti-cancer therapy is administered in combination with a second anti-cancer therapy or agent, such as any of the anti-cancer agents or therapies described herein. In some embodiments, the second anti-cancer therapy or agent comprises a PRMT5-targeted therapy. In some embodiments, the anti-cancer therapy comprises a PTEN-targeted therapy and a PRMT5-targeted therapy. In some embodiments, the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

Also provided herein are methods of treating or delaying progression of a cancer in an individual, such as a cancer provided herein, e.g., a UCB. In some embodiments, the individual has a cancer comprising a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the methods of treating or delaying progression of a cancer of the disclosure, e.g., a UCB comprising a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, comprise administering to the individual a therapeutically effective amount of an anti-cancer therapy, such as an anti-cancer therapy provided herein. In some embodiments, the methods of treating or delaying progression of a cancer of the disclosure, e.g., a UCB comprising a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, comprise administering to the individual an effective amount of an anti-cancer therapy, such as an anti-cancer therapy provided herein, responsive to knowledge of the presence of the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual. In some embodiments, the sample is a sample described herein. In some embodiments, the sample comprises cells from the cancer or is obtained from cells from the cancer. The methods of treatment disclosed herein may include any of the anti-cancer therapies and/or therapeutic agents described herein, e.g., infra.

In some embodiments, the methods of treating or delaying progression of a cancer of the disclosure, e.g., a UCB comprising a deletion of an MTAP gene, or of a portion thereof, and one or more mutations in an FGFR3 gene or in a PTEN gene, comprise administering to the individual a therapeutically effective amount of a PRMT5-targeted therapy and an additional anti-cancer therapy, e.g., an FGFR-targeted therapy or a PTEN-targeted therapy. In some embodiments, the methods of treating or delaying progression of a cancer of the disclosure, e.g., a UCB comprising a deletion of an MTAP gene, or of a portion thereof, and one or more mutations in an FGFR3 gene or in a PTEN gene, comprise administering to the individual an effective amount of a PRMT5-targeted therapy and an additional anti-cancer therapy, e.g., an FGFR-targeted therapy or a PTEN-targeted therapy, responsive to knowledge of the presence of the deletion of an MTAP gene, or of a portion thereof, and one or more mutations in an FGFR3 gene or in a PTEN gene in a sample obtained from the individual.

In some embodiments, the methods of treating or delaying progression of a cancer of the disclosure, e.g., a UCB comprising a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, comprise administering to the individual a therapeutically effective amount of a PRMT5-targeted therapy and an additional anti-cancer therapy, e.g., an anti-cancer therapy provided herein. In some embodiments, the methods of treating or delaying progression of a cancer of the disclosure, e.g., a UCB comprising a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, comprise administering to the individual an effective amount of a PRMT5-targeted therapy and an additional anti-cancer therapy, e.g., an anti-cancer therapy provided herein, responsive to knowledge of the presence of the deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from the individual.

In some embodiments, the anti-cancer therapy is administered in combination with a second anti-cancer therapy or agent, such as any of the anti-cancer agents or therapies described herein. In some embodiments, the second anti-cancer therapy or agent comprises a PRMT5-targeted therapy. In some embodiments, the anti-cancer therapy comprises any of the anti-cancer therapies provided herein and a PRMT5-targeted therapy. In some embodiments, the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

In some embodiments of any of the methods provided herein, the methods further comprise acquiring knowledge of or detecting in a sample from an individual a deletion of a CDKN2A gene, or of a portion thereof; and/or a deletion of a CDKN2B gene, or of a portion thereof. In some embodiments of any of the methods provided herein, the individual has a deletion of a CDKN2A gene, or of a portion thereof; and/or a deletion of a CDKN2B gene, or of a portion thereof. In some embodiments of any of the methods provided herein, the individual has a heterozygous or a homozygous deletion of a CDKN2A gene, or of a portion thereof; and/or a heterozygous or a homozygous deletion of a CDKN2B gene, or of a portion thereof. In some embodiments of any of the methods provided herein, the individual has a heterozygous or a homozygous deletion of the MTAP gene, or of a portion thereof.

Reporting

In some embodiments, the methods provided herein comprise generating a report, and/or providing a report to party.

In some embodiments, a report according to the present disclosure comprises information about one or more of: a deletion of an MTAP gene, or of a portion thereof, and one or more mutations in an FGFR3 gene or in a PTEN gene; a cancer of the disclosure (e.g., a UCB), e.g., comprising a deletion of an MTAP gene, or of a portion thereof, and one or more mutations in an FGFR3 gene or in a PTEN gene; or a treatment, a therapy, or one or more treatment options (e.g., an FGFR-targeted therapy or a PTEN-targeted therapy) for an individual having a cancer, such as a cancer of the disclosure (e.g., a UCB, e.g., comprising a deletion of an MTAP gene, or of a portion thereof, and one or more mutations in an FGFR3 gene or in a PTEN gene). In some embodiments, a report according to the present disclosure comprises information about one or more of: a deletion of an MTAP gene, or of a portion thereof, and one or more mutations in an FGFR3 gene; a cancer of the disclosure (e.g., a UCB), e.g., comprising a deletion of an MTAP gene, or of a portion thereof, and one or more mutations in an FGFR3 gene; or a treatment, a therapy, or one or more treatment options (e.g., an FGFR-targeted therapy) for an individual having a cancer, such as a cancer of the disclosure (e.g., a UCB, e.g., comprising a deletion of an MTAP gene, or of a portion thereof, and one or more mutations in an FGFR3 gene). In some embodiments, a report according to the present disclosure comprises information about one or more of: a deletion of an MTAP gene, or of a portion thereof, and one or more mutations in a PTEN gene; a cancer of the disclosure (e.g., a UCB), e.g., comprising a deletion of an MTAP gene, or of a portion thereof, and one or more mutations in a PTEN gene; or a treatment, a therapy, or one or more treatment options (e.g., a PTEN-targeted therapy) for an individual having a cancer, such as a cancer of the disclosure (e.g., a UCB, e.g., comprising a deletion of an MTAP gene, or of a portion thereof, and one or more mutations in a PTEN gene). In some embodiments, the report further comprises information about one or more of: a deletion of a CDKN2A gene, or of a portion thereof; and/or a deletion of a CDKN2B gene, or of a portion thereof, e.g., a heterozygous or a homozygous deletion of a CDKN2A gene, or of a portion thereof; and/or a heterozygous or a homozygous deletion of a CDKN2B gene, or of a portion thereof. In some embodiments, the report further comprises information about one or more of: one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1.

In some embodiments, a report according to the present disclosure comprises information about the presence or absence of a deletion of an MTAP gene, or of a portion thereof, and one or more mutations in an FGFR3 gene or in a PTEN gene in a sample obtained from an individual, such as an individual having a cancer, e.g., a cancer provided herein, e.g., a UCB. In one embodiment, a report according to the present disclosure indicates that a deletion of an MTAP gene, or of a portion thereof, and one or more mutations in an FGFR3 gene or in a PTEN gene are present in a sample obtained from the individual. In one embodiment, a report according to the present disclosure indicates that a deletion of an MTAP gene, or of a portion thereof, and one or more mutations in an FGFR3 gene or in a PTEN gene are not present in a sample obtained from the individual. In one embodiment, a report according to the present disclosure indicates that a deletion of an MTAP gene, or of a portion thereof, and one or more mutations in an FGFR3 gene or in a PTEN gene have been detected in a sample obtained from the individual. In one embodiment, a report according to the present disclosure indicates that a deletion of an MTAP gene, or of a portion thereof, and one or more mutations in an FGFR3 gene or in a PTEN gene have not been detected in a sample obtained from the individual. In some embodiments, the report comprises an identifier for the individual from which the sample was obtained. In some embodiments, a report according to the present disclosure further comprises information about the presence or absence of a deletion of a CDKN2A gene, or of a portion thereof, and/or a deletion of a CDKN2B gene, or of a portion thereof, in a sample obtained from an individual, such as an individual having a cancer, e.g., a cancer provided herein, e.g., a UCB. In some embodiments, a report according to the present disclosure further comprises information about the presence or absence of one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual, such as an individual having a cancer, e.g., a cancer provided herein, e.g., a UCB. In one embodiment, a report according to the present disclosure further indicates that a deletion of a CDKN2A gene, or of a portion thereof, and/or a deletion of a CDKN2B gene, or of a portion thereof, are present in a sample obtained from the individual. In one embodiment, a report according to the present disclosure further indicates that one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 are present in a sample obtained from the individual. In one embodiment, a report according to the present disclosure further indicates that a deletion of a CDKN2A gene, or of a portion thereof, and/or a deletion of a CDKN2B gene, or of a portion thereof, are not present in a sample obtained from the individual. In one embodiment, a report according to the present disclosure further indicates that one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, orBAPI are not present in a sample obtained from the individual. In one embodiment, a report according to the present disclosure further indicates that a deletion of a CDKN2A gene, or of a portion thereof, and/or a deletion of a CDKN2B gene, or of a portion thereof, have been detected in a sample obtained from the individual. In one embodiment, a report according to the present disclosure further indicates that one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, orBAPI have been detected in a sample obtained from the individual. In one embodiment, a report according to the present disclosure further indicates that a deletion of a CDKN2A gene, or of a portion thereof, and/or a deletion of a CDKN2B gene, or of a portion thereof, have not been detected in a sample obtained from the individual. In one embodiment, a report according to the present disclosure further indicates that one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 have not been detected in a sample obtained from the individual. In some embodiments, the report comprises an identifier for the individual from which the sample was obtained.

In some embodiments, the report includes information on the role of a deletion of an MTAP gene, or of a portion thereof, and one or more mutations in an FGFR3 gene or in a PTEN gene, or of the corresponding wild type counterparts, in disease, such as in cancer, e.g., a UCB. Such information can include one or more of: information on prognosis of a cancer, such as a cancer provided herein, e.g., comprising a deletion of an MTAP gene, or of a portion thereof, and one or more mutations in an FGFR3 gene or in a PTEN gene; information on resistance of a cancer, such as a cancer provided herein, e.g., a UCB, such as a UCB comprising a deletion of an MTAP gene, or of a portion thereof, and one or more mutations in an FGFR3 gene or in a PTEN gene, to one or more treatments; information on potential or suggested therapeutic options (e.g., such as an anti-cancer therapy provided herein, or a treatment selected or identified according to the methods provided herein, e.g., an FGFR-targeted therapy or a PTEN-targeted therapy); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein, e.g., an FGFR-targeted therapy or PTEN-targeted therapy) to an individual having a cancer, such as a cancer provided herein (e.g., a UCB), e.g., comprising a deletion of an MTAP gene, or of a portion thereof, and one or more mutations in an FGFR3 gene or in a PTEN gene, and identified in the report. In some embodiments, the report includes information or a recommendation on the administration of a treatment (e.g., an anti-cancer therapy provided herein, or a treatment selected or identified according to the methods provided herein, such as an FGFR-targeted therapy or a PTEN-targeted therapy). In some embodiments, the report further includes information on the role of a deletion of a CDKN2A gene, or of a portion thereof, and/or a deletion of a CDKN2B gene, or of a portion thereof, or of the corresponding wild type counterparts, in disease, such as in cancer, e.g., a UCB. In some embodiments, the report further includes information on the role of one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, or of the corresponding wild type counterparts, in disease, such as in cancer, e.g., a UCB. Such information can include one or more of: information on prognosis of a cancer, such as a cancer provided herein, e.g., comprising a deletion of a CDKN2A gene, or of a portion thereof, and/or a deletion of a CDKN2B gene, or of a portion thereof; information on resistance of a cancer, such as a cancer provided herein, e.g., a UCB, such as a UCB comprising a deletion of a CDKN2A gene, or of a portion thereof, and/or a deletion of a CDKN2B gene, or of a portion thereof, to one or more treatments; information on potential or suggested therapeutic options (e.g., such as an anti-cancer therapy provided herein, or a treatment selected or identified according to the methods provided herein, e.g., an FGFR-targeted therapy or a PTEN-targeted therapy); or information on therapeutic options that should be avoided. Such information can include one or more of: information on prognosis of a cancer, such as a cancer provided herein, e.g., comprising one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1; information on resistance of a cancer, such as a cancer provided herein, e.g., a UCB, such as a UCB comprising one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein to an individual having a cancer, such as a cancer provided herein (e.g., a UCB), e.g., comprising a deletion of a CDKN2A gene, or of a portion thereof, and/or a deletion of a CDKN2B gene, or of a portion thereof, and identified in the report. In some embodiments, the report includes information on the likely effectiveness, acceptability, and/or advisability of applying a therapeutic option (e.g., such as an anti-cancer therapy provided herein, or a treatment selected or identified according to the methods provided herein to an individual having a cancer, such as a cancer provided herein (e.g., a UCB), e.g., comprising one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, and identified in the report. In some embodiments, the report includes information or a recommendation on the administration of a treatment (e.g., an anti-cancer therapy provided herein, or a treatment selected or identified according to the methods provided herein, such as an FGFR-targeted therapy or a PTEN-targeted therapy). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein (e.g., a UCB); detecting a deletion of an MTAP gene, or of a portion thereof, and one or more mutations in an FGFR3 gene or in a PTEN gene in the sample, or acquiring knowledge of the presence of a deletion of an MTAP gene, or of a portion thereof, and one or more mutations in an FGFR3 gene or in a PTEN gene in the sample; and generating a report. In some embodiments, the method further comprises detecting a deletion of a CDKN2A gene, or of a portion thereof, and/or a deletion of a CDKN2B gene, or of a portion thereof, in the sample, or acquiring knowledge of the presence of a deletion of a CDKN2A gene, or of a portion thereof, and/or a deletion of a CDKN2B gene, or of a portion thereof, in the sample. In some embodiments, the method comprises detecting one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample, or acquiring knowledge of the presence of one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample. In some embodiments, a report generated according to the methods provided herein comprises one or more of: information about the presence or absence of a deletion of an MTAP gene, or of a portion thereof, and one or more mutations in an FGFR3 gene or in a PTEN gene in the sample; an identifier for the individual from which the sample was obtained; information on the role of a deletion of an MTAP gene, or of a portion thereof, and one or more mutations in an FGFR3 gene or in a PTEN gene, or the corresponding wild type counterparts, in disease (e.g., such as in cancer, e.g., a UCB); information on prognosis, resistance, or potential or suggested therapeutic options (such as an anti-cancer therapy provided herein, or a treatment selected or identified according to the methods provided herein, such as an FGFR-targeted therapy or a PTEN-targeted therapy); information on the likely effectiveness, acceptability, or the advisability of applying a therapeutic option (such as an anti-cancer therapy provided herein, or a treatment selected or identified according to the methods provided herein, e.g., an FGFR-targeted therapy or a PTEN-targeted therapy) to the individual; a recommendation or information on the administration of a treatment (such as an anti-cancer therapy provided herein, or a treatment selected or identified according to the methods provided herein, e.g., an FGFR-targeted therapy or a PTEN-targeted therapy); or a recommendation or information on the dosage or treatment regimen of a treatment (such as an anti-cancer therapy provided herein, or a treatment selected or identified according to the methods provided herein, e.g., an FGFR-targeted therapy or a PTEN-targeted therapy), e.g., in combination with other treatments (e.g., a second therapeutic agent). In some embodiments, the report further comprises information about the presence or absence of a deletion of a CDKN2A gene, or of a portion thereof, and/or a deletion of a CDKN2B gene, or of a portion thereof, in the sample. In some embodiments, the report comprises information about the presence or absence of one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in the sample. In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., a UCB, such as a UCB comprising a deletion of an MTAP gene, or of a portion thereof, and one or more mutations in an FGFR3 gene or in a PTEN gene), and optionally a deletion of a CDKN2A gene, or of a portion thereof, and/or a deletion of a CDKN2B gene, or of a portion thereof, or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a deletion of an MTAP gene, or of a portion thereof, and one or more mutations in an FGFR3 gene or in a PTEN gene, and optionally a deletion of a CDKN2A gene, or of a portion thereof, and/or a deletion of a CDKN2B gene, or of a portion thereof, in a sample obtained from an individual (e.g., an individual having a cancer, e.g., a UCB). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a deletion of an MTAP gene, or of a portion thereof, and one or more mutations in an FGFR3 gene or in a PTEN gene, and optionally a deletion of a CDKN2A gene, or of a portion thereof, and/or a deletion of a CDKN2B gene, or of a portion thereof, in a sample obtained from an individual (e.g., an individual having a cancer). The report may be provided to an individual or a patient (e.g., an individual or a patient with a cancer, such as a cancer provided herein, e.g., a UCB, such as a UCB comprising a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual (e.g., an individual having a cancer, e.g., a UCB). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 in a sample obtained from an individual (e.g., an individual having a cancer).

In some embodiments of any of the reports or methods provided herein, the potential or suggested therapeutic options include a PRMT5-targeted therapy and an additional anti-cancer therapy, e.g., an anti-cancer therapy provided herein, an FGFR-targeted therapy or a PTEN-targeted therapy. In some embodiments, the report includes information on the likely effectiveness, acceptability, and/or advisability of applying a PRMT5-targeted therapy and an additional anti-cancer therapy, e.g., an anti-cancer therapy provided herein, an FGFR-targeted therapy or a PTEN-targeted therapy, to an individual having a cancer, such as a cancer provided herein (e.g., a UCB), e.g., comprising a deletion of an MTAP gene, or of a portion thereof, and one or more mutations in an FGFR3 gene or in a PTEN gene, and identified in the report. In some embodiments, the report includes information or a recommendation on the administration of a PRMT5-targeted therapy and an additional anti-cancer therapy, e.g., an FGFR-targeted therapy or a PTEN-targeted therapy. In some embodiments, the report includes information on the likely effectiveness, acceptability, and/or advisability of applying a PRMT5-targeted therapy and an additional anti-cancer therapy, e.g., an anti-cancer therapy provided herein, to an individual having a cancer, such as a cancer provided herein (e.g., a UCB), e.g., comprising a deletion of an MTAP gene, or of a portion thereof, and one or more mutations in one or more genes selected from CDKN2A, CDKN2B, TERT, TP53, KDM6A, PIK3CA, KMT2D, CCND1, FGF19, FGF4, FGF3, ARID1A, TSC1, ERBB2, STAG2, MDM2, CREBBP, EP300, CDKN1A, ATM, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, and identified in the report. In some embodiments, the report includes information or a recommendation on the administration of a PRMT5-targeted therapy and an additional anti-cancer therapy, e.g., an anti-cancer therapy provided herein.

### MTAP-Intact Urothelial Bladder Cancer

In some aspects, provided herein are methods of treating or delaying progression of a urothelial bladder cancer (UCB) in an individual, wherein the UCB does not comprise a deletion of an methylthioadenosine phosphorylase (MTAP) gene, or of a portion thereof (MTAP-intact UCB).

In other aspects, methods are provided for identifying one or more treatment options for an individual having an MTAP-intact UCB. In other aspects, methods are provided for selecting treatment for an individual having an MTAP-intact UCB. In some aspects, methods are provided for predicting survival of an individual having an MTAP-intact UCB treated with an anti-cancer therapy.

In other aspects, provided herein are methods of identifying an individual having an MTAP-intact UCB, who may benefit from a treatment comprising an anti-cancer therapy. In other aspects, provided herein are methods of selecting a therapy for an individual having an MTAP-intact UCB. In other aspects, provided herein are methods of assessing, screening, or diagnosing an individual having an MTAP-intact UCB.

In some embodiments, the methods comprise acquiring knowledge of or detecting in a sample from an individual having an MTAP-intact UCB a tumor mutational burden (TMB) of at least about 10 mut/Mb or at least about 20 mut/Mb, a PD-L1-positive UCB, or one or more mutations in an RB1 gene. In some embodiments, the methods comprise acquiring knowledge of or detecting in a sample from an individual having an MTAP-intact UCB, a tumor mutational burden (TMB) of at least about 10 mut/Mb or at least about 20 mut/Mb, a PD-L1-positive UCB, or one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2.

Methods of Selecting or Identifying a Treatment

In some aspects, provided herein are methods of identifying an individual having cancer, e.g., a UCB, who may benefit from an anti-cancer therapy, e.g., an anti-cancer therapy provided herein. In some embodiments, the methods comprise detecting a TMB of at least about 10 mut/Mb or at least about 20 mut/Mb, a PD-L1-positive status, and/or one or more mutations in an RB1 gene in a sample obtained from the individual. In some embodiments, the methods comprise acquiring knowledge of the presence of a TMB of at least about 10 mut/Mb or at least about 20 mut/Mb, a PD-L1-positive status, and/or one or more mutations in an RB1 gene in a sample obtained from the individual. In some embodiments, the presence of the TMB of at least about 10 mut/Mb or at least about 20 mut/Mb, a PD-L1-positive status, and/or one or more mutations in an RB1 gene in the sample identifies the individual as one who may benefit from a treatment comprising an anti-cancer therapy, e.g., an anti-cancer therapy provided herein, such as an immunotherapy or an RB1-targeted therapy. In some embodiments, detection of the TMB of at least about 10 mut/Mb or at least about 20 mut/Mb, the PD-L1-positive status, and/or one or more mutations in an RB1 gene in the sample identifies the individual as one who may benefit from a treatment comprising an anti-cancer therapy, e.g., an anti-cancer therapy provided herein, such as an immunotherapy or an RB1-targeted therapy. In some embodiments, responsive to knowledge of a TMB of at least about 10 mut/Mb or at least about 20 mut/Mb, a PD-L1-positive status, and/or one or more mutations in an RB1 gene in the sample, the individual is identified as one who may benefit from a treatment comprising an anti-cancer therapy, e.g., an anti-cancer therapy provided herein, such as an immunotherapy or an RB1-targeted therapy. In some embodiments, the presence of a TMB of at least about 10 mut/Mb or at least about 20 mut/Mb and/or a PD-L1-positive status in the sample identifies the individual as one who may benefit from a treatment comprising an anti-cancer therapy, e.g., an anti-cancer therapy provided herein, such as an immunotherapy. In some embodiments, detection of a TMB of at least about 10 mut/Mb or at least about 20 mut/Mb and/or a PD-L1-positive status in the sample identifies the individual as one who may benefit from a treatment comprising an anti-cancer therapy, e.g., an anti-cancer therapy provided herein, such as an immunotherapy. In some embodiments, responsive to knowledge of the TMB of at least about 10 mut/Mb or at least about 20 mut/Mb and/or a PD-L1-positive status in the sample, the individual is identified as one who may benefit from a treatment comprising an anti-cancer therapy, e.g., an anti-cancer therapy provided herein, such as an immunotherapy. In some embodiments, the presence of one or more mutations in an RB1 gene in the sample identifies the individual as one who may benefit from a treatment comprising an anti-cancer therapy, e.g., an anti-cancer therapy provided herein, such as an RB1-targeted therapy. In some embodiments, detection of one or more mutations in an RB1 gene in the sample identifies the individual as one who may benefit from a treatment comprising an anti-cancer therapy, e.g., an anti-cancer therapy provided herein, such as an RB1-targeted therapy. In some embodiments, responsive to knowledge of one or more mutations in an RB1 gene in the sample, the individual is identified as one who may benefit from a treatment comprising an anti-cancer therapy, e.g., an anti-cancer therapy provided herein, such as an RB1-targeted therapy. In some embodiments, the TMB of at least about 10 mut/Mb or at least about 20 mut/Mb, the PD-L1-positive status, and/or one or more mutations in an RB1 gene are detected using any suitable method known in the art or described herein. In some embodiments, the sample is a sample described herein. In some embodiments, the sample comprises cells from the cancer or is obtained from cells from the cancer, e.g., a UCB.

Also provided herein are methods of identifying or selecting a treatment, a therapy, or one or more treatment options for an individual having a cancer, such as a cancer described herein, e.g., a UCB. In some embodiments, the cancer comprises a TMB of at least about 10 mut/Mb or at least about 20 mut/Mb, a PD-L1-positive status, and/or one or more mutations in an RB1 gene. In some embodiments, the methods comprise detecting a TMB of at least about 10 mut/Mb or at least about 20 mut/Mb, a PD-L1-positive status, and/or one or more mutations in an RB1 gene in a sample obtained from an individual having a cancer, such as a cancer described herein, e.g., a UCB. In some embodiments, the TMB of at least about 10 mut/Mb or at least about 20 mut/Mb, the PD-L1-positive status, and/or one or more mutations in an RB1 gene are detected using any suitable method known in the art or described herein. In some embodiments, detection of a TMB of at least about 10 mut/Mb or at least about 20 mut/Mb, a PD-L1-positive status, and/or one or more mutations in an RB1 gene in a sample obtained from an individual having a cancer, e.g., a cancer described herein, e.g., a UCB, identifies the individual as one who may benefit from an anti-cancer therapy, e.g., an anti-cancer therapy provided herein, such as an immunotherapy or an RB1-targeted therapy. In some embodiments, the presence of a TMB of at least about 10 mut/Mb or at least about 20 mut/Mb, a PD-L1-positive status, and/or one or more mutations in an RB1 gene in a sample obtained from an individual having a cancer, e.g., a cancer provided herein, e.g., a UCB, identifies the individual as one who may benefit from an anti-cancer therapy, such as an anti-cancer therapy provided herein, e.g., an immunotherapy or an RB1-targeted therapy. In some embodiments, responsive to detection of a TMB of at least about 10 mut/Mb or at least about 20 mut/Mb, a PD-L1-positive status, and/or one or more mutations in an RB1 gene in a sample obtained from an individual having a cancer, e.g., a cancer provided herein, e.g., a UCB, the individual is classified as a candidate to receive an anti-cancer therapy, such as an anti-cancer therapy provided herein, e.g., an immunotherapy or an RB1-targeted therapy. In some embodiments, responsive to detection of a TMB of at least about 10 mut/Mb or at least about 20 mut/Mb, a PD-L1-positive status, and/or one or more mutations in an RB1 gene in a sample obtained from an individual having a cancer, e.g., a cancer provided herein, e.g., a UCB, the individual is classified or identified as likely to respond to an anti-cancer therapy, such as an anti-cancer therapy provided herein, e.g., an immunotherapy or an RB1-targeted therapy. In some embodiments, detection of a TMB of at least about 10 mut/Mb or at least about 20 mut/Mb and/or a PD-L1-positive status in a sample obtained from an individual having a cancer, e.g., a cancer described herein, e.g., a UCB, identifies the individual as one who may benefit from an anti-cancer therapy, e.g., an anti-cancer therapy provided herein, such as an immunotherapy. In some embodiments, the presence of a TMB of at least about 10 mut/Mb or at least about 20 mut/Mb and/or a PD-L1-positive status in a sample obtained from an individual having a cancer, e.g., a cancer provided herein, e.g., a UCB, identifies the individual as one who may benefit from an anti-cancer therapy, such as an anti-cancer therapy provided herein, e.g., an immunotherapy. In some embodiments, responsive to detection of a TMB of at least about 10 mut/Mb or at least about 20 mut/Mb and/or a PD-L1-positive status in a sample obtained from an individual having a cancer, e.g., a cancer provided herein, e.g., a UCB, the individual is classified as a candidate to receive an anti-cancer therapy, such as an anti-cancer therapy provided herein, e.g., an immunotherapy. In some embodiments, responsive to detection of a TMB of at least about 10 mut/Mb or at least about 20 mut/Mb and/or a PD-L1-positive status in a sample obtained from an individual having a cancer, e.g., a cancer provided herein, e.g., a UCB, the individual is classified or identified as likely to respond to an anti-cancer therapy, such as an anti-cancer therapy provided herein, e.g., an immunotherapy. In some embodiments, detection of one or more mutations in an RB1 gene in a sample obtained from an individual having a cancer, e.g., a cancer described herein, e.g., a UCB, identifies the individual as one who may benefit from an anti-cancer therapy, e.g., an anti-cancer therapy provided herein, such as an RB1-targeted therapy. In some embodiments, the presence of one or more mutations in an RB1 gene in a sample obtained from an individual having a cancer, e.g., a cancer provided herein, e.g., a UCB, identifies the individual as one who may benefit from an anti-cancer therapy, such as an anti-cancer therapy provided herein, e.g., an RB1-targeted therapy. In some embodiments, responsive to detection of one or more mutations in an RB1 gene in a sample obtained from an individual having a cancer, e.g., a cancer provided herein, e.g., a UCB, the individual is classified as a candidate to receive an anti-cancer therapy, such as an anti-cancer therapy provided herein, e.g., an RB1-targeted therapy. In some embodiments, responsive to detection of one or more mutations in an RB1 gene in a sample obtained from an individual having a cancer, e.g., a cancer provided herein, e.g., a UCB, the individual is classified or identified as likely to respond to an anti-cancer therapy, such as an anti-cancer therapy provided herein, e.g., an RB1-targeted therapy. In some embodiments, the sample is a sample described herein. In some embodiments, the sample comprises cells from the cancer or is obtained from cells from the cancer, e.g., a UCB. In some embodiments, the methods further comprise generating a report, e.g., as described herein. In some embodiments, the report comprises a treatment, a therapy, or one or more treatment options identified or selected for the individual, e.g., based at least in part on detection of a TMB of at least about 10 mut/Mb or at least about 20 mut/Mb, a PD-L1-positive status, and/or one or more mutations in an RB1 gene in the sample.

In some aspects, provided herein are methods of identifying an individual having cancer, e.g., a UCB, who may benefit from an anti-cancer therapy, e.g., an anti-cancer therapy provided herein. In some embodiments, the methods comprise detecting a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual. In some embodiments, the methods comprise acquiring knowledge of the presence of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual. In some embodiments, the presence of the TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample identifies the individual as one who may benefit from a treatment comprising an anti-cancer therapy, e.g., an anti-cancer therapy provided herein, such as an immunotherapy and/or another anti-cancer therapy provided herein. In some embodiments, detection of the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample identifies the individual as one who may benefit from a treatment comprising an anti-cancer therapy, e.g., an anti-cancer therapy provided herein, such as an immunotherapy and/or another anti-cancer therapy provided herein. In some embodiments, responsive to knowledge of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample, the individual is identified as one who may benefit from a treatment comprising an anti-cancer therapy, e.g., an anti-cancer therapy provided herein, such as an immunotherapy and/or an anti-cancer therapy provided herein. In some embodiments, the presence of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample identifies the individual as one who may benefit from a treatment comprising an anti-cancer therapy, e.g., an anti-cancer therapy provided herein. In some embodiments, detection of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample identifies the individual as one who may benefit from a treatment comprising an anti-cancer therapy, e.g., an anti-cancer therapy provided herein. In some embodiments, responsive to knowledge of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample, the individual is identified as one who may benefit from a treatment comprising an anti-cancer therapy, e.g., an anti-cancer therapy provided herein. In some embodiments, the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 are detected using any suitable method known in the art or described herein. In some embodiments, the sample is a sample described herein. In some embodiments, the sample comprises cells from the cancer or is obtained from cells from the cancer, e.g., a UCB.

Also provided herein are methods of identifying or selecting a treatment, a therapy, or one or more treatment options for an individual having a cancer, such as a cancer described herein. In some embodiments, the cancer comprises a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the methods comprise detecting a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual having a cancer, such as a cancer described herein, e.g., a UCB. In some embodiments, the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 are detected using any suitable method known in the art or described herein. In some embodiments, detection of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual having a cancer, e.g., a cancer described herein, e.g., a UCB, identifies the individual as one who may benefit from an anti-cancer therapy, e.g., an anti-cancer therapy provided herein, such as an immunotherapy and/or another anti-cancer therapy provided herein. In some embodiments, the presence of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual having a cancer, e.g., a cancer provided herein, e.g., a UCB, identifies the individual as one who may benefit from an anti-cancer therapy, such as an anti-cancer therapy provided herein, e.g., an immunotherapy and/or another anti-cancer therapy provided herein. In some embodiments, responsive to detection of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual having a cancer, e.g., a cancer provided herein, e.g., a UCB, the individual is classified as a candidate to receive an anti-cancer therapy, such as an anti-cancer therapy provided herein, e.g., an immunotherapy and/or another anti-cancer therapy provided herein. In some embodiments, responsive to detection of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual having a cancer, e.g., a cancer provided herein, e.g., a UCB, the individual is classified or identified as likely to respond to an anti-cancer therapy, such as an anti-cancer therapy provided herein, e.g., an immunotherapy and/or another anti-cancer therapy provided herein. In some embodiments, detection of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual having a cancer, e.g., a cancer described herein, e.g., a UCB, identifies the individual as one who may benefit from an anti-cancer therapy, e.g., an anti-cancer therapy provided herein. In some embodiments, the presence of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual having a cancer, e.g., a cancer provided herein, e.g., a UCB, identifies the individual as one who may benefit from an anti-cancer therapy, such as an anti-cancer therapy provided herein. In some embodiments, responsive to detection of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual having a cancer, e.g., a cancer provided herein, e.g., a UCB, the individual is classified as a candidate to receive an anti-cancer therapy, such as an anti-cancer therapy provided herein. In some embodiments, responsive to detection of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual having a cancer, e.g., a cancer provided herein, e.g., a UCB, the individual is classified or identified as likely to respond to an anti-cancer therapy, such as an anti-cancer therapy provided herein. In some embodiments, the sample is a sample described herein. In some embodiments, the sample comprises cells from the cancer or is obtained from cells from the cancer, e.g., a UCB. In some embodiments, the methods further comprise generating a report, e.g., as described herein. In some embodiments, the report comprises a treatment, a therapy, or one or more treatment options identified or selected for the individual, e.g., based at least in part on detection of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample.

In some embodiments of any of the methods provided herein, the methods further comprise acquiring knowledge of or detecting in a sample from an individual the absence of a deletion of an MTAP gene, or of a portion thereof. In some embodiments of any of the methods provided herein, the individual does not have a deletion of an MTAP gene, or of a portion thereof.

Methods of Treatment

Also provided herein are methods of treating or delaying progression of a cancer in an individual, such as a cancer provided herein, e.g., a UCB. In some embodiments, the individual has a cancer comprising a TMB of at least about 10 mut/Mb or at least about 20 mut/Mb, a PD-L1-positive status, and/or one or more mutations in an RB1 gene. In some embodiments, the methods of treating or delaying progression of a cancer of the disclosure, e.g., a UCB, comprising a TMB of at least about 10 mut/Mb or at least about 20 mut/Mb, a PD-L1-positive status, and/or one or more mutations in an RB1 gene, comprise administering to an individual a therapeutically effective amount of an anti-cancer therapy, such as an anti-cancer therapy provided herein, e.g., an immunotherapy or an RB1-targeted therapy. In some embodiments, the methods of treating or delaying progression of a cancer of the disclosure, e.g., a UCB, comprising a TMB of at least about 10 mut/Mb or at least about 20 mut/Mb, a PD-L1-positive status, and/or one or more mutations in an RB1 gene, comprise administering to an individual an effective amount of an anti-cancer therapy, such as an anti-cancer therapy provided herein (e.g., an immunotherapy or an RB1-targeted therapy), responsive to knowledge of the presence of the TMB of at least about 10 mut/Mb or at least about 20 mut/Mb, the PD-L1-positive status, and/or one or more mutations in an RB1 gene in a sample obtained from the individual. In some embodiments, the sample is a sample described herein. In some embodiments, the sample comprises cells from the cancer or is obtained from cells from the cancer, e.g. a UCB. The methods of treatment disclosed herein may include any of the anti-cancer therapies and/or therapeutic agents described herein, e.g., infra.

In some embodiments, the individual has a cancer comprising a TMB of at least about 10 mut/Mb or at least about 20 mut/Mb and/or a PD-L1-positive status. In some embodiments, the methods of treating or delaying progression of a cancer of the disclosure, e.g., a UCB, comprising a TMB of at least about 10 mut/Mb or at least about 20 mut/Mb and/or a PD-L1-positive status, comprise administering to an individual a therapeutically effective amount of an anti-cancer therapy, such as an anti-cancer therapy provided herein, e.g., an immunotherapy. In some embodiments, the methods of treating or delaying progression of a cancer of the disclosure, e.g., a UCB comprising a TMB of at least about 10 mut/Mb or at least about 20 mut/Mb and/or a PD-L1-positive status, comprise administering to an individual an effective amount of an anti-cancer therapy, such as an anti-cancer therapy provided herein (e.g., an immunotherapy), responsive to knowledge of the presence of the TMB of at least about 10 mut/Mb or at least about 20 mut/Mb and/or the PD-L1-positive status in a sample obtained from the individual. In some embodiments, the immunotherapy comprises one or more of a checkpoint inhibitor (e.g., a checkpoint inhibitor provided herein), a cancer vaccine (e.g., a cancer vaccine provided herein), a cell-based therapy (e.g., a cell-based therapy provided herein), a T cell receptor (TCR)-based therapy (e.g., a TCR-based therapy provided herein), an adjuvant immunotherapy (e.g., an adjuvant immunotherapy provided herein), a cytokine immunotherapy (e.g., a cytokine immunotherapy provided herein), or an oncolytic virus therapy (e.g., an oncolytic virus therapy provided herein). In some embodiments, the anti-cancer therapy is administered in combination with a second anti-cancer therapy or agent, such as any of the anti-cancer agents or therapies described herein.

In some embodiments, the individual has a cancer comprising one or more mutations in an RB1 gene. In some embodiments, the methods of treating or delaying progression of a cancer of the disclosure, e.g., a UCB comprising one or more mutations in an RB1 gene, comprise administering to an individual a therapeutically effective amount of an anti-cancer therapy, such as an anti-cancer therapy provided herein, e.g., an RB1-targeted therapy. In some embodiments, the methods of treating or delaying progression of a cancer of the disclosure, e.g., a UCB comprising one or more mutations in an RB1 gene, comprise administering to the individual an effective amount of an anti-cancer therapy, such as an anti-cancer therapy provided herein (e.g., an RB1-targeted therapy), responsive to knowledge of the presence of the one or more mutations in an RB1 gene in a sample obtained from the individual. In some embodiments, the RB1-targeted therapy comprises one or more of a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor. In some embodiments, the anti-cancer therapy is administered in combination with a second anti-cancer therapy or agent, such as any of the anti-cancer agents or therapies described herein.

Also provided herein are methods of treating or delaying progression of a cancer in an individual, such as a cancer provided herein, e.g., a UCB. In some embodiments, the individual has a cancer comprising a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the methods of treating or delaying progression of a cancer of the disclosure, e.g., a UCB comprising a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, comprise administering to an individual a therapeutically effective amount of an anti-cancer therapy, such as an anti-cancer therapy provided herein, e.g., an immunotherapy and/or another anti-cancer therapy provided herein. In some embodiments, the methods of treating or delaying progression of a cancer of the disclosure, e.g., a UCB comprising a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, comprise administering to an individual an effective amount of an anti-cancer therapy, such as an anti-cancer therapy provided herein (e.g., an immunotherapy or another anti-cancer therapy provided herein), responsive to knowledge of the presence of the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual. In some embodiments, the sample is a sample described herein. In some embodiments, the sample comprises cells from the cancer or is obtained from cells from the cancer, e.g. a UCB. The methods of treatment disclosed herein may include any of the anti-cancer therapies and/or therapeutic agents described herein, e.g., infra.

In some embodiments, the individual has a cancer comprising one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the methods of treating or delaying progression of a cancer of the disclosure, e.g., a UCB comprising one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, comprise administering to an individual a therapeutically effective amount of an anti-cancer therapy, such as an anti-cancer therapy provided herein. In some embodiments, the methods of treating or delaying progression of a cancer of the disclosure, e.g., a UCB comprising one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, comprise administering to the individual an effective amount of an anti-cancer therapy, such as an anti-cancer therapy provided herein, responsive to knowledge of the presence of the one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual. In some embodiments, the anti-cancer therapy is administered in combination with a second anti-cancer therapy or agent, such as any of the anti-cancer agents or therapies described herein.

In some embodiments of any of the methods provided herein, the methods further comprise acquiring knowledge of or detecting in a sample from an individual the absence of a deletion of an MTAP gene, or of a portion thereof. In some embodiments of any of the methods provided herein, the individual does not have a deletion of an MTAP gene, or of a portion thereof.

Methods of Diagnosing, Assessing, Screening, Monitoring or Predicting

In some aspects, provided herein are methods of diagnosing or assessing a TMB of at least about 10 mut/Mb or at least about 20 mut/Mb, a PD-L1-positive status, and/or one or more mutations in an RB1 gene in a sample from an individual having a cancer, such as a UCB. In some embodiments, the methods comprise acquiring knowledge of the presence of a TMB of at least about 10 mut/Mb or at least about 20 mut/Mb, a PD-L1-positive status, and/or one or more mutations in an RB1 gene in a sample obtained from the individual. In some embodiments, the methods comprise detecting a TMB of at least about 10 mut/Mb or at least about 20 mut/Mb, a PD-L1-positive status, and/or one or more mutations in an RB1 gene in a sample obtained from the individual. In some embodiments, a TMB of at least about 10 mut/Mb or at least about 20 mut/Mb, a PD-L1-positive status, and/or one or more mutations in an RB1 gene are detected in a sample obtained from the individual using any method known in the art, such as one or more of the methods of detection of nucleic acid molecules or polypeptides described herein. In some embodiments, the methods further comprise providing a diagnosis or an assessment of the TMB of at least about 10 mut/Mb or at least about 20 mut/Mb, the PD-L1-positive status, and/or one or more mutations in an RB1 gene. In some embodiments, the diagnosis or assessment identifies the presence or absence of a TMB of at least about 10 mut/Mb or at least about 20 mut/Mb, a PD-L1-positive status, and/or one or more mutations in an RB1 gene in the sample. In some embodiments, the diagnosis or assessment identifies the cancer, such as a UCB provided herein, as likely to respond to an anti-cancer therapy, e.g., an anti-cancer therapy provided herein, such as an immunotherapy and/or an RB1-targeted therapy. In some embodiments, the presence of a TMB of at least about 10 mut/Mb or at least about 20 mut/Mb, a PD-L1-positive status, and/or one or more mutations in an RB1 gene in the sample identifies the cancer as likely to respond to an anti-cancer therapy, e.g., an anti-cancer therapy provided herein, such as an immunotherapy or an RB1-targeted therapy. In some embodiments, the diagnosis or assessment identifies the presence or absence of a TMB of at least about 10 mut/Mb or at least about 20 mut/Mb and/or a PD-L1-positive status in the sample. In some embodiments, the diagnosis or assessment identifies the cancer, such as a UCB provided herein, as likely to respond to an anti-cancer therapy, e.g., an anti-cancer therapy provided herein, such as an immunotherapy. In some embodiments, the presence of a TMB of at least about 10 mut/Mb or at least about 20 mut/Mb and/or a PD-L1-positive status in the sample identifies the cancer as likely to respond to an anti-cancer therapy, e.g., an anti-cancer therapy provided herein, such as an immunotherapy. In some embodiments, the diagnosis or assessment identifies the presence or absence of one or more mutations in an RB1 gene in the sample. In some embodiments, the diagnosis or assessment identifies the cancer, such as a UCB provided herein, as likely to respond to an anti-cancer therapy, e.g., an anti-cancer therapy provided herein, such as an RB1-targeted therapy. In some embodiments, the presence of one or more mutations in an RB1 gene in the sample identifies the cancer as likely to respond to an anti-cancer therapy, e.g., an anti-cancer therapy provided herein, such as an RB1-targeted therapy. In some embodiments, the sample is a sample described herein. In some embodiments, the sample comprises cells from the cancer or is obtained from cells from the cancer, e.g., from a UCB. In some embodiments, the individual has a cancer, is suspected of having a cancer, is being tested for a cancer, is being treated for a cancer, or is being tested for a susceptibility to a cancer, e.g., a UCB.

In some aspects, provided herein are methods of diagnosing or assessing a cancer in an individual, e.g., a UCB. In some embodiments, the methods of diagnosing or assessing cancer comprise detecting a TMB of at least about 10 mut/Mb or at least about 20 mut/Mb, a PD-L1-positive status, and/or one or more mutations in an RB1 gene in a sample obtained from the individual, e.g., a sample comprising cells from the cancer, e.g., a UCB. In some embodiments, the methods comprise detecting a TMB of at least about 10 mut/Mb or at least about 20 mut/Mb, a PD-L1-positive status, and/or one or more mutations in an RB1 gene in a sample obtained from the individual using any method known in the art, such as one or more of the methods of detection of nucleic acid molecules or polypeptides described herein. In some embodiments, detection of a TMB of at least about 10 mut/Mb or at least about 20 mut/Mb, a PD-L1-positive status, and/or one or more mutations in an RB1 gene in a sample obtained from the individual identifies the cancer, e.g., the UCB, as likely to respond to an anti-cancer therapy, e.g., an anti-cancer therapy provided herein, such as an immunotherapy or an RB1-targeted therapy. In some embodiments, the presence of a TMB of at least about 10 mut/Mb or at least about 20 mut/Mb, a PD-L1-positive status, and/or one or more mutations in an RB1 gene in a sample obtained from the individual identifies the cancer, e.g., the UCB, as likely to respond to an anti-cancer therapy, e.g., an anti-cancer therapy provided herein, such as an immunotherapy or an RB1-targeted therapy. In some embodiments, the methods further comprise providing a diagnosis or an assessment of the cancer, e.g., the UCB, or of the TMB of at least about 10 mut/Mb or at least about 20 mut/Mb, a PD-L1-positive status, and/or one or more mutations in an RB1 gene. In some embodiments, the diagnosis or assessment identifies the cancer as likely to respond to an anti-cancer therapy, e.g., an anti-cancer therapy provided herein, such as an immunotherapy or an RB1-targeted therapy. In some embodiments, detection of a TMB of at least about 10 mut/Mb or at least about 20 mut/Mb and/or a PD-L1-positive status in a sample obtained from the individual identifies the cancer, e.g., the UCB, as likely to respond to an anti-cancer therapy, e.g., an anti-cancer therapy provided herein, such as an immunotherapy. In some embodiments, the presence of a TMB of at least about 10 mut/Mb or at least about 20 mut/Mb and/or a PD-L1-positive status in a sample obtained from the individual identifies the cancer, e.g., the UCB, as likely to respond to an anti-cancer therapy, e.g., an anti-cancer therapy provided herein, such as an immunotherapy. In some embodiments, the methods further comprise providing a diagnosis or an assessment of the cancer, e.g., the UCB, or of the TMB of at least about 10 mut/Mb or at least about 20 mut/Mb and/or a PD-L1-positive status. In some embodiments, the diagnosis or assessment identifies the cancer as likely to respond to an anti-cancer therapy, e.g., an anti-cancer therapy provided herein, such as an immunotherapy. In some embodiments, detection of one or more mutations in an RB1 gene in a sample obtained from the individual identifies the cancer, e.g., the UCB, as likely to respond to an anti-cancer therapy, e.g., an anti-cancer therapy provided herein, such as an RB1-targeted therapy. In some embodiments, the presence of one or more mutations in an RB1 gene in a sample obtained from the individual identifies the cancer, e.g., the UCB, as likely to respond to an anti-cancer therapy, e.g., an anti-cancer therapy provided herein, such as an RB1-targeted therapy. In some embodiments, the methods further comprise providing a diagnosis or an assessment of the cancer, e.g., the UCB, or of the one or more mutations in an RB1 gene. In some embodiments, the diagnosis or assessment identifies the cancer as likely to respond to an anti-cancer therapy, e.g., an anti-cancer therapy provided herein, such as an RB1-targeted therapy. In some embodiments, the diagnosis or assessment identifies the presence or absence of the TMB of at least about 10 mut/Mb or at least about 20 mut/Mb, the PD-L1-positive status, and/or one or more mutations in an RB1 gene in the sample.

In some aspects, provided herein are methods of predicting survival of an individual having a cancer, e.g., a cancer provided herein, e.g., a UCB. In some embodiments, the individual is being treated with an anti-cancer therapy, such as an anti-cancer therapy described herein. In some embodiments, the methods comprise acquiring knowledge of a TMB of at least about 10 mut/Mb or at least about 20 mut/Mb, a PD-L1-positive status, and/or one or more mutations in an RB1 gene in a sample from the individual. In some embodiments, the methods comprise detecting a TMB of at least about 10 mut/Mb or at least about 20 mut/Mb, a PD-L1-positive status, and/or one or more mutations in an RB1 gene in a sample from the individual. In some embodiments, responsive to acquiring knowledge of a TMB of at least about 10 mut/Mb or at least about 20 mut/Mb, a PD-L1-positive status, and/or one or more mutations in an RB1 gene in the sample, the individual is predicted to have longer survival after treatment with an anti-cancer therapy, e.g., an anti-cancer therapy provided herein (e.g., an immunotherapy or an RB1-targeted therapy), for example, as compared to an individual whose cancer does not exhibit the TMB of at least about 10 mut/Mb or at least about 20 mut/Mb, the PD-L1-positive status, and/or one or more mutations in an RB1 gene. In some embodiments, responsive to detecting a TMB of at least about 10 mut/Mb or at least about 20 mut/Mb, a PD-L1-positive status, and/or one or more mutations in an RB1 gene in the sample, the individual is predicted to have longer survival after treatment with an anti-cancer therapy, e.g., an anti-cancer therapy provided herein (e.g., an immunotherapy or an RB1-targeted therapy), for example, as compared to an individual whose cancer does not exhibit the TMB of at least about 10 mut/Mb or at least about 20 mut/Mb, the PD-L1-positive status, and/or one or more mutations in an RB1 gene. In some embodiments, responsive to acquiring knowledge of a TMB of at least about 10 mut/Mb or at least about 20 mut/Mb and/or a PD-L1-positive status in the sample, the individual is predicted to have longer survival after treatment with an anti-cancer therapy, e.g., an anti-cancer therapy provided herein (e.g., an immunotherapy), for example, as compared to an individual whose cancer does not exhibit the TMB of at least about 10 mut/Mb or at least about 20 mut/Mb and/or the PD-L1-positive status. In some embodiments, responsive to detecting a TMB of at least about 10 mut/Mb or at least about 20 mut/Mb and/or a PD-L1-positive status in the sample, the individual is predicted to have longer survival after treatment with an anti-cancer therapy, e.g., an anti-cancer therapy provided herein (e.g., an immunotherapy), for example, as compared to an individual whose cancer does not exhibit the TMB of at least about 10 mut/Mb or at least about 20 mut/Mb and/or the PD-L1-positive status. In some embodiments, responsive to acquiring knowledge of one or more mutations in an RB1 gene in the sample, the individual is predicted to have longer survival after treatment with an anti-cancer therapy, e.g., an anti-cancer therapy provided herein (e.g., an RB1-targeted therapy), for example, as compared to an individual whose cancer does not exhibit the one or more mutations in an RB1 gene. In some embodiments, responsive to detecting one or more mutations in an RB1 gene in the sample, the individual is predicted to have longer survival after treatment with an anti-cancer therapy, e.g., an anti-cancer therapy provided herein (e.g., an RB1-targeted therapy), for example, as compared to an individual whose cancer does not exhibit the one or more mutations in an RB1 gene. In some embodiments, the methods further comprise providing a diagnosis or an assessment. In some embodiments, the diagnosis or assessment identifies the presence or absence of the TMB of at least about 10 mut/Mb or at least about 20 mut/Mb, the PD-L1-positive status, and/or one or more mutations in an RB1 gene in the sample. In some embodiments, the diagnosis or assessment identifies the individual as being predicted to have longer survival after treatment with an anti-cancer therapy, e.g., an anti-cancer therapy provided herein (e.g., an immunotherapy or an RB1-targeted therapy), for example, as compared to an individual whose cancer does not exhibit the TMB of at least about 10 mut/Mb or at least about 20 mut/Mb, the PD-L1-positive status, and/or one or more mutations in an RB1 gene. In some embodiments, the diagnosis or assessment identifies the individual as being predicted to have longer survival after treatment with an anti-cancer therapy, e.g., an anti-cancer therapy provided herein (e.g., an immunotherapy), for example, as compared to an individual whose cancer does not exhibit the TMB of at least about 10 mut/Mb or at least about 20 mut/Mb and/or the PD-L1-positive status. In some embodiments, the diagnosis or assessment identifies the individual as being predicted to have longer survival after treatment with an anti-cancer therapy, e.g., an anti-cancer therapy provided herein (e.g., an RB1-targeted therapy), for example, as compared to an individual whose cancer does not exhibit the one or more mutations in an RB1 gene. In some embodiments, the sample is a sample described herein. In some embodiments, the sample comprises cells from the cancer or is obtained from cells from the cancer, e.g., the UCB.

In some aspects, provided herein are methods of screening an individual having cancer, suspected of having cancer, being tested for cancer, being treated for cancer, or being tested for a susceptibility to cancer, e.g., a UCB. In some embodiments, the individual is being treated with an anti-cancer therapy, such as an anti-cancer therapy described herein. In some embodiments, the methods comprise acquiring knowledge of a TMB of at least about 10 mut/Mb or at least about 20 mut/Mb, a PD-L1-positive status, and/or one or more mutations in an RB1 gene in a sample from the individual. In some embodiments, the methods comprise detecting a TMB of at least about 10 mut/Mb or at least about 20 mut/Mb, a PD-L1-positive status, and/or one or more mutations in an RB1 gene in a sample from the individual. In some embodiments, responsive to acquiring knowledge of a TMB of at least about 10 mut/Mb or at least about 20 mut/Mb, a PD-L1-positive status, and/or one or more mutations in an RB1 gene in the sample, the individual is predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, the PD-L1-positive status, and/or one or more mutations in an RB1 gene. In some embodiments, responsive to detecting a TMB of at least about 10 mut/Mb or at least about 20 mut/Mb, a PD-L1-positive status, and/or one or more mutations in an RB1 gene in the sample, the individual is predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, a PD-L1-positive status, and/or one or more mutations in an RB1 gene. In some embodiments, the methods further comprise providing a diagnosis or an assessment. In some embodiments, the diagnosis or assessment identifies the presence or absence of the TMB of at least about 10 mut/Mb or at least about 20 mut/Mb, the PD-L1-positive status, and/or one or more mutations in an RB1 gene in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, the PD-L1-positive status, and/or one or more mutations in an RB1 gene. In some embodiments, the sample is a sample described herein. In some embodiments, the sample comprises cells from the cancer or is obtained from cells from the cancer, e.g., the UCB.

In some embodiments, the methods further comprise selectively enriching for one or more nucleic acids comprising nucleotide sequences that comprise one or more mutations in an RB1 gene to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise one or more mutations in an RB1 gene to produce an enriched sample, e.g., using a reagent known in the art or provided herein, such as an antibody described herein.

In some aspects, provided herein are methods of diagnosing or assessing a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample from an individual having a cancer, such as a UCB. In some embodiments, the methods comprise acquiring knowledge of the presence of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual. In some embodiments, the methods comprise detecting a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual. In some embodiments, a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 are detected in a sample obtained from the individual using any method known in the art, such as one or more of the methods of detection of nucleic acid molecules or polypeptides described herein. In some embodiments, the methods further comprise providing a diagnosis or an assessment of the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the diagnosis or assessment identifies the presence or absence of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample. In some embodiments, the diagnosis or assessment identifies the cancer, such as a UCB provided herein, as likely to respond to an anti-cancer therapy, e.g., an anti-cancer therapy provided herein, such as an immunotherapy and/or another anti-cancer therapy provided herein. In some embodiments, the presence of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample identifies the cancer as likely to respond to an anti-cancer therapy, e.g., an anti-cancer therapy provided herein, such as an immunotherapy and/or another anti-cancer therapy provided herein. In some embodiments, the diagnosis or assessment identifies the presence or absence of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample. In some embodiments, the diagnosis or assessment identifies the cancer, such as a UCB provided herein, as likely to respond to an anti-cancer therapy, e.g., an anti-cancer therapy provided herein. In some embodiments, the presence of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample identifies the cancer as likely to respond to an anti-cancer therapy, e.g., an anti-cancer therapy provided herein. In some embodiments, the sample is a sample described herein. In some embodiments, the sample comprises cells from the cancer or is obtained from cells from the cancer, e.g., from a UCB. In some embodiments, the individual has a cancer, is suspected of having a cancer, is being tested for a cancer, is being treated for a cancer, or is being tested for a susceptibility to a cancer, e.g., a UCB.

In some aspects, provided herein are methods of diagnosing or assessing a cancer in an individual, e.g., a UCB. In some embodiments, the methods of diagnosing or assessing cancer comprise detecting a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual, e.g., a sample comprising cells from the cancer, e.g., a UCB. In some embodiments, the methods comprise detecting a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual using any method known in the art, such as one or more of the methods of detection of nucleic acid molecules or polypeptides described herein. In some embodiments, detection of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual identifies the cancer, e.g., the UCB, as likely to respond to an anti-cancer therapy, e.g., an anti-cancer therapy provided herein, such as an immunotherapy and/or another anti-cancer therapy provided herein. In some embodiments, the presence of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual identifies the cancer, e.g., the UCB, as likely to respond to an anti-cancer therapy, e.g., an anti-cancer therapy provided herein, such as an immunotherapy and/or another anti-cancer therapy provided herein. In some embodiments, the methods further comprise providing a diagnosis or an assessment of the cancer, e.g., the UCB, or of the TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the diagnosis or assessment identifies the cancer as likely to respond to an anti-cancer therapy, e.g., an anti-cancer therapy provided herein, such as an immunotherapy and/or another anti-cancer therapy provided herein. In some embodiments, detection of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual identifies the cancer, e.g., the UCB, as likely to respond to an anti-cancer therapy, e.g., an anti-cancer therapy provided herein. In some embodiments, the presence of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from the individual identifies the cancer, e.g., the UCB, as likely to respond to an anti-cancer therapy, e.g., an anti-cancer therapy provided herein. In some embodiments, the methods further comprise providing a diagnosis or an assessment of the cancer, e.g., the UCB, or of the one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the diagnosis or assessment identifies the cancer as likely to respond to an anti-cancer therapy, e.g., an anti-cancer therapy provided herein. In some embodiments, the diagnosis or assessment identifies the presence or absence of the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample.

In some aspects, provided herein are methods of predicting survival of an individual having a cancer, e.g., a cancer provided herein, e.g., a UCB. In some embodiments, the individual is being treated with an anti-cancer therapy, such as an anti-cancer therapy described herein. In some embodiments, the methods comprise acquiring knowledge of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample from the individual. In some embodiments, the methods comprise detecting a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample from the individual. In some embodiments, responsive to acquiring knowledge of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample, the individual is predicted to have longer survival after treatment with an anti-cancer therapy, e.g., an anti-cancer therapy provided herein (e.g., an immunotherapy and/or another anti-cancer therapy provided herein), for example, as compared to an individual whose cancer does not exhibit the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, responsive to detecting a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample, the individual is predicted to have longer survival after treatment with an anti-cancer therapy, e.g., an anti-cancer therapy provided herein (e.g., an immunotherapy and/or another anti-cancer therapy provided herein), for example, as compared to an individual whose cancer does not exhibit the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, responsive to acquiring knowledge of one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample, the individual is predicted to have longer survival after treatment with an anti-cancer therapy, e.g., an anti-cancer therapy provided herein, for example, as compared to an individual whose cancer does not exhibit the one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, responsive to detecting one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample, the individual is predicted to have longer survival after treatment with an anti-cancer therapy, e.g., an anti-cancer therapy provided herein, for example, as compared to an individual whose cancer does not exhibit the one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the methods further comprise providing a diagnosis or an assessment. In some embodiments, the diagnosis or assessment identifies the presence or absence of the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample. In some embodiments, the diagnosis or assessment identifies the individual as being predicted to have longer survival after treatment with an anti-cancer therapy, e.g., an anti-cancer therapy provided herein (e.g., an immunotherapy and/or another anti-cancer therapy provided herein), for example, as compared to an individual whose cancer does not exhibit the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the sample is a sample described herein. In some embodiments, the sample comprises cells from the cancer or is obtained from cells from the cancer, e.g., the UCB.

In some aspects, provided herein are methods of screening an individual having cancer, suspected of having cancer, being tested for cancer, being treated for cancer, or being tested for a susceptibility to cancer, e.g., a UCB. In some embodiments, the individual is being treated with an anti-cancer therapy, such as an anti-cancer therapy described herein. In some embodiments, the methods comprise acquiring knowledge of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample from the individual. In some embodiments, the methods comprise detecting a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample from the individual. In some embodiments, responsive to acquiring knowledge of a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample, the individual is predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, responsive to detecting a TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample, the individual is predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, a PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the methods further comprise providing a diagnosis or an assessment. In some embodiments, the diagnosis or assessment identifies the presence or absence of the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, the PD-L1-positive status, and/or one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the sample is a sample described herein. In some embodiments, the sample comprises cells from the cancer or is obtained from cells from the cancer, e.g., the UCB.

In some embodiments, the methods further comprise selectively enriching for one or more nucleic acids comprising nucleotide sequences that comprise one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise one or more mutations in one or more genes selected from TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 to produce an enriched sample, e.g., using a reagent known in the art or provided herein, such as an antibody described herein.

In some embodiments of any of the methods provided herein, the methods further comprise acquiring knowledge of or detecting in a sample from an individual the absence of a deletion of an MTAP gene, or of a portion thereof. In some embodiments of any of the methods provided herein, the individual does not have a deletion of an MTAP gene, or of a portion thereof.

Reporting

In some embodiments, the methods provided herein comprise generating a report, and/or providing a report to party.

In some embodiments, a report according to the present disclosure comprises information about one or more of: a TMB of at least about 10 mut/Mb or at least about 20 mut/Mb, a PD-L1-positive status, and/or one or more mutations in an RB1 gene; a cancer of the disclosure (e.g., a UCB), e.g., comprising a TMB of at least about 10 mut/Mb or at least about 20 mut/Mb, a PD-L1-positive status, and/or one or more mutations in an RB1 gene; or a treatment, a therapy, or one or more treatment options (e.g., an immunotherapy or an RB1-targeted therapy) for an individual having a cancer, such as a cancer of the disclosure (e.g., a UCB, e.g., comprising a TMB of at least about 10 mut/Mb or at least about 20 mut/Mb, a PD-L1-positive status, and/or one or more mutations in an RB1 gene). In some embodiments, a report according to the present disclosure comprises information about one or more of: a TMB of at least about 10 mut/Mb or at least about 20 mut/Mb and/or a PD-L1-positive status; a cancer of the disclosure (e.g., a UCB), e.g., comprising a TMB of at least about 10 mut/Mb or at least about 20 mut/Mb and/or a PD-L1-positive status; or a treatment, a therapy, or one or more treatment options (e.g., an immunotherapy) for an individual having a cancer, such as a cancer of the disclosure (e.g., a UCB, e.g., comprising a TMB of at least about 10 mut/Mb or at least about 20 mut/Mb and/or a PD-L1-positive status). In some embodiments, a report according to the present disclosure comprises information about one or more of: one or more mutations in an RB1 gene; a cancer of the disclosure (e.g., a UCB), e.g., comprising one or more mutations in an RB1 gene; or a treatment, a therapy, or one or more treatment options (e.g., an RB1-targeted therapy) for an individual having a cancer, such as a cancer of the disclosure (e.g., a UCB, e.g., comprising one or more mutations in an RB1 gene). In some embodiments, a report according to the present disclosure further comprises information about the absence of a deletion of an MTAP gene, or of a portion thereof, in the sample. In some embodiments, a report according to the present disclosure comprises information about the presence of one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample.

In some embodiments, a report according to the present disclosure comprises information about the presence or absence of a TMB of at least about 10 mut/Mb or at least about 20 mut/Mb, a PD-L1-positive status, and/or one or more mutations in an RB1 gene in a sample obtained from an individual, such as an individual having a cancer, e.g., a cancer provided herein, e.g., a UCB. In some embodiments, a report according to the present disclosure further comprises information about the absence of a deletion of an MTAP gene, or of a portion thereof, in the sample. In some embodiments, a report according to the present disclosure comprises information about the presence of one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample. In one embodiment, a report according to the present disclosure indicates that a TMB of at least about 10 mut/Mb or at least about 20 mut/Mb, a PD-L1-positive status, and/or one or more mutations in an RB1 gene are present in a sample obtained from the individual. In one embodiment, a report according to the present disclosure indicates that a deletion of an MTAP gene, or of a portion thereof, is not present in the sample. In one embodiment, a report according to the present disclosure indicates that one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 are not present in the sample. In one embodiment, a report according to the present disclosure indicates that one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 are present in the sample. In one embodiment, a report according to the present disclosure indicates that a TMB of at least about 10 mut/Mb or at least about 20 mut/Mb, a PD-L1-positive status, and/or one or more mutations in an RB1 gene are not present in a sample obtained from the individual. In one embodiment, a report according to the present disclosure indicates that a TMB of at least about 10 mut/Mb or at least about 20 mut/Mb, a PD-L1-positive status, and/or one or more mutations in an RB1 gene have been detected in a sample obtained from the individual. In one embodiment, a report according to the present disclosure indicates that a TMB of at least about 10 mut/Mb or at least about 20 mut/Mb, a PD-L1-positive status, and/or one or more mutations in an RB1 gene have not been detected in a sample obtained from the individual. In one embodiment, a report according to the present disclosure further indicates that a deletion of an MTAP gene, or of a portion thereof, has not been detected in the sample. In one embodiment, a report according to the present disclosure indicates that one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 have not been detected in the sample. In one embodiment, a report according to the present disclosure further indicates that one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 have been detected in the sample. In some embodiments, the report comprises an identifier for the individual from which the sample was obtained.

In some embodiments, the report includes information on the role of a TMB of at least about 10 mut/Mb or at least about 20 mut/Mb, a PD-L1-positive status, and/or one or more mutations in an RB1 gene, or of the corresponding wild type counterparts, in disease, such as in cancer, e.g., a UCB. In some embodiments, the report further includes information on the role of the absence of a deletion of an MTAP gene, or of a portion thereof, in disease, such as in cancer, e.g., a UCB. In some embodiments, the report further includes information on the role of one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in disease, such as in cancer, e.g., a UCB. Such information can include one or more of: information on prognosis of a cancer, such as a cancer provided herein, e.g., comprising a TMB of at least about 10 mut/Mb or at least about 20 mut/Mb, a PD-L1-positive status, and/or one or more mutations in an RB1 gene, and/or not having a deletion of an MTAP gene, or of a portion thereof; information on resistance of a cancer, such as a cancer provided herein, e.g., a UCB, such as a UCB comprising a TMB of at least about 10 mut/Mb or at least about 20 mut/Mb, a PD-L1-positive status, and/or one or more mutations in an RB1 gene, and/or not having a deletion of an MTAP gene, or of a portion thereof, to one or more treatments; information on potential or suggested therapeutic options (e.g., such as an anti-cancer therapy provided herein, or a treatment selected or identified according to the methods provided herein, e.g., an immunotherapy or an RB1-targeted therapy); or information on therapeutic options that should be avoided. Such information can include one or more of: information on prognosis of a cancer, such as a cancer provided herein, e.g., comprising one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2; information on resistance of a cancer, such as a cancer provided herein, e.g., a UCB, such as a UCB comprising one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein, e.g., an immunotherapy or a PTEN-targeted therapy) to an individual having a cancer, such as a cancer provided herein (e.g., a UCB), e.g., comprising a TMB of at least about 10 mut/Mb or at least about 20 mut/Mb, a PD-L1-positive status, and/or one or more mutations in an RB1 gene, and/or not having a deletion of an MTAP gene, or of a portion thereof, and identified in the report. In some embodiments, the report includes information or a recommendation on the administration of a treatment (e.g., an anti-cancer therapy provided herein, or a treatment selected or identified according to the methods provided herein, such as an immunotherapy or an RB1-targeted therapy). In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein (e.g., a UCB), e.g., comprising one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, and identified in the report. In some embodiments, the report includes information or a recommendation on the administration of a treatment (e.g., an anti-cancer therapy provided herein, or a treatment selected or identified according to the methods provided herein, such as an immunotherapy or an RB1-targeted therapy). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein (e.g., a UCB); detecting a TMB of at least about 10 mut/Mb or at least about 20 mut/Mb, a PD-L1-positive status, and/or one or more mutations in an RB1 gene in the sample, or acquiring knowledge of the presence of a TMB of at least about 10 mut/Mb or at least about 20 mut/Mb, a PD-L1-positive status, and/or one or more mutations in an RB1 gene in the sample; and generating a report. In some embodiments, the method further comprises detecting the absence of a deletion of an MTAP gene, or of a portion thereof, in the sample, or acquiring knowledge of the absence of a deletion of an MTAP gene, or of a portion thereof, in the sample. In some embodiments, the method comprises detecting one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample, or acquiring knowledge of one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample. In some embodiments, a report generated according to the methods provided herein comprises one or more of: information about the presence or absence of a TMB of at least about 10 mut/Mb or at least about 20 mut/Mb, a PD-L1-positive status, and/or one or more mutations in an RB1 gene in the sample; an identifier for the individual from which the sample was obtained; information on the role of a TMB of at least about 10 mut/Mb or at least about 20 mut/Mb, a PD-L1-positive status, and/or one or more mutations in an RB1 gene, or the corresponding wild type counterparts, in disease (e.g., such as in cancer, e.g., a UCB); information on prognosis, resistance, or potential or suggested therapeutic options (such as an anti-cancer therapy provided herein, or a treatment selected or identified according to the methods provided herein, such as an immunotherapy or an RB1-targeted therapy); information on the likely effectiveness, acceptability, or the advisability of applying a therapeutic option (such as an anti-cancer therapy provided herein, or a treatment selected or identified according to the methods provided herein, e.g., an immunotherapy or an RB1-targeted therapy) to the individual; a recommendation or information on the administration of a treatment (such as an anti-cancer therapy provided herein, or a treatment selected or identified according to the methods provided herein, e.g., an immunotherapy or an RB1-targeted therapy); or a recommendation or information on the dosage or treatment regimen of a treatment (such as an anti-cancer therapy provided herein, or a treatment selected or identified according to the methods provided herein, e.g., an immunotherapy or an RB1-targeted therapy), e.g., in combination with other treatments (e.g., a second therapeutic agent). In some embodiments, a report generated according to the methods provided herein comprises one or more of: information about the presence or absence one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in the sample; an identifier for the individual from which the sample was obtained; information on the role of one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, or the corresponding wild type counterparts, in disease (e.g., such as in cancer, e.g., a UCB); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, a report generated according to the methods provided herein further comprises information about the absence of a deletion of an MTAP gene, or of a portion thereof in the sample. In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., a UCB, such as a UCB comprising a TMB of at least about 10 mut/Mb or at least about 20 mut/Mb, a PD-L1-positive status, and/or one or more mutations in an RB1 gene, and/or optionally not having a deletion of an MTAP gene, or of a portion thereof), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a TMB of at least about 10 mut/Mb or at least about 20 mut/Mb, a PD-L1-positive status, and/or one or more mutations in an RB1 gene, and/or optionally the absence of a deletion of an MTAP gene, or of a portion thereof, in a sample obtained from an individual (e.g., an individual having a cancer, e.g., a UCB). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a TMB of at least about 10 mut/Mb or at least about 20 mut/Mb, a PD-L1-positive status, and/or one or more mutations in an RB1 gene, and/or optionally of a deletion of an MTAP gene, or of a portion thereof, in a sample obtained from an individual (e.g., an individual having a cancer). The report may be provided to an individual or a patient (e.g., an individual or a patient with a cancer, such as a cancer provided herein, e.g., a UCB, such as a UCB comprising one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual (e.g., an individual having a cancer, e.g., a UCB). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2 in a sample obtained from an individual (e.g., an individual having a cancer).

In some embodiments of any of the methods provided herein, a sample from an individual, e.g., an individual having a UCB, is determined to be PD-L1-negative if 0% of tumor cells in the sample express PD-L1. In some embodiments of any of the methods provided herein, a sample from an individual, e.g., an individual having a UCB, is determined to be PD-L1 positive if at least about 1% of tumor cells in the sample express PD-L1. In some embodiments of any of the methods provided herein, a sample from an individual, e.g., an individual having a UCB, is determined to be PD-L1 low positive if between about 1% and about 49% of tumor cells in the sample express PD-L1. In some embodiments of any of the methods provided herein, a sample from an individual, e.g., an individual having a UCB, is determined to be PD-L1 high positive if at least about 50% or more of tumor cells in the sample express PD-L1. In some embodiments of any of the methods provided herein, a sample from an individual, e.g., an individual having a UCB, is determined to be PD-L1 positive if the sample is PD-L1 low positive or PD-L1 high positive.

Non-Small Cell Lung Cancer

Non-small cell lung cancer (NSCLC) is one of the most common types of cancer. NSCLC arises from cells within the lungs, but more advanced NSCLC can metastasize to other tissues. NSCLC in a subject can be classified into one of several subtypes based on the type of cells and structures affected. For instance, the NSCLC can be an adenocarcinoma, a squamous cell carcinoma (also known as epidermoid carcinoma), a large cell carcinoma (such as a large cell neuroendocrine carcinoma), a sarcomatoid carcinoma, an adenosquamous carcinoma, or a NSCLC not otherwise specified (NOS).

In some embodiments, a NSCLC of the disclosure contains a deletion in the MTAP gene (e.g., a deletion of the entire MTAP gene, or of a portion of the MTAP gene). The MTAP gene encodes a methyl-thioadenosine phosphorylase that plays a major role in the metabolism of polyamines such as adenosine and methionine. In some embodiments, the deletion in the MTAP gene is a deletion of a portion of thereof. In some embodiments, the deletion in the MTAP gene is a deletion of the entire MTAP gene. In some embodiments, the deletion in the MTAP gene is a heterozygous deletion. In other embodiments, the deletion in the MTAP gene is a homozygous deletion. In some embodiments, the deletion is a deletion at the 9p21 locus. In some embodiments, a NSCLC of the disclosure contains a deletion of a CDKN2A gene or a CDKN2B gene. In some embodiments, the deletion of the CDKN2A gene or the CDKN2B gene is a deletion of a portion of thereof. In some embodiments, the deletion of the CDKN2A gene or the CDKN2B gene is a deletion of the entire CDKN2A gene or CDKN2B gene. In some embodiments, the deletion of the CDKN2A gene or the CDKN2B gene is a heterozygous deletion. In other embodiments, the deletion of the CDKN2A gene or the CDKN2B gene is a homozygous deletion. In some embodiments, the deletion is a deletion at the 9p21 locus. In some embodiments, a NSCLC of the disclosure contains a co-deletion of an MTAP gene, or of a portion thereof, and of a CDKN2A gene and/or a CDKN2B gene, or portions thereof, e.g., at the 9p21 locus.

In some embodiments, a NSCLC of the disclosure contains a deletion in the MTAP gene (e.g., a deletion of the entire MTAP gene, or of a portion of the MTAP gene) and one or more mutations in one or more genes, wherein the one or more genes comprise CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, a NSCLC of the disclosure contains a deletion in the MTAP gene (e.g., a deletion of the entire MTAP gene, or of a portion of the MTAP gene) and one or more mutations in the EGFR gene. In some embodiments, the NSCLC contains a deletion in the MTAP gene (e.g., a deletion of the entire MTAP gene, or of a portion of the MTAP gene) and one or more mutations in the SMARCA4 gene.

In some embodiments, a NSCLC of the disclosure contains an intact MTAP gene (e.g., lacks a deletion of an MTAP gene, or of a portion thereof). In some embodiments, a NSCLC of the disclosure contains an intact MTAP gene and is PD-L1-positive. In some embodiments, a NSCLC of the disclosure contains an intact MTAP gene and has a tumor mutational burden (TMB) of at least about 10 mut/Mb or at least about 20 mut/Mb. In some embodiments, a NSCLC of the disclosure contains an intact MTAP gene and one or more mutations in one or more genes, wherein the one or more genes comprise CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, a NSCLC of the disclosure contains an intact MTAP gene and one or more mutations in an RB1 gene, a KRAS gene, or a TP53 gene.

### MTAP-Deleted Non-Small Cell Lung Cancer

In some aspects, provided herein are methods of treating or delaying progression of a non-small cell lung cancer (NSCLC) in an individual, wherein the NSCLC comprises a deletion of an methylthioadenosine phosphorylase (MTAP) gene, or of a portion thereof (MTAP-deleted NSCLC).

In other aspects, methods are provided for identifying one or more treatment options for an individual having an MTAP-deleted NSCLC. In other aspects, methods are provided for selecting treatment for an individual having an MTAP-deleted NSCLC. In some aspects, methods are provided for predicting survival of an individual having an MTAP-deleted NSCLC treated with an anti-cancer therapy. In other aspects, provided herein are methods of identifying an individual having an MTAP-deleted NSCLC, who may benefit from a treatment comprising an anti-cancer therapy. In other aspects, provided herein are methods of selecting a therapy for an individual having an MTAP-deleted NSCLC. In other aspects, provided herein are methods of assessing, screening, or diagnosing an individual having an MTAP-deleted NSCLC.

In some embodiments, the methods comprise acquiring knowledge of or detecting in a sample from an individual having an MTAP-deleted NSCLC a deletion of an MTAP gene, or of a portion thereof, and one or more mutations in one or more mutations in an EGFR gene or in a SMARCA4 gene. In some embodiments, the methods comprise acquiring knowledge of or detecting in a sample from an individual having an MTAP-deleted NSCLC a deletion of an MTAP gene, or of a portion thereof, and one or more mutations in one or more genes, wherein the one or more genes comprise CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1.

Methods of Selecting or Identifying a Treatment

In some aspects, provided herein are methods of identifying an individual having cancer, e.g., a NSCLC, who may benefit from an anti-cancer therapy, e.g., an anti-cancer therapy provided herein. In some embodiments, the methods comprise detecting a deletion of an MTAP gene, or of a portion thereof, and one or more mutations in an EGFR gene or in a SMARCA4 gene in a sample obtained from the individual. In some embodiments, the methods comprise acquiring knowledge of the presence of a deletion of an MTAP gene, or of a portion thereof, and one or more mutations in an EGFR gene or in a SMARCA4 gene in a sample obtained from the individual. In some embodiments, the presence of the deletion of an MTAP gene, or of a portion thereof, and one or more mutations in an EGFR gene or in a SMARCA4 gene in the sample identifies the individual as one who may benefit from a treatment comprising an anti-cancer therapy, e.g., an anti-cancer therapy provided herein, such as an EGFR-targeted therapy or a SMARCA4-targeted therapy. In some embodiments, detection of the deletion of an MTAP gene, or of a portion thereof, and one or more mutations in an EGFR gene or in a SMARCA4 gene in the sample identifies the individual as one who may benefit from a treatment comprising an anti-cancer therapy, e.g., an anti-cancer therapy provided herein, such as an EGFR-targeted therapy or a SMARCA4-targeted therapy. In some embodiments, responsive to knowledge of the deletion of an MTAP gene, or of a portion thereof, and one or more mutations in an EGFR gene or in a SMARCA4 gene in the sample, the individual is identified as one who may benefit from a treatment comprising an anti-cancer therapy, e.g., an anti-cancer therapy provided herein, such as an EGFR-targeted therapy or a SMARCA4-targeted therapy. In some embodiments, the presence of the deletion of an MTAP gene, or of a portion thereof, and one or more mutations in an EGFR gene in the sample identifies the individual as one who may benefit from a treatment comprising an anti-cancer therapy, e.g., an anti-cancer therapy provided herein, such as an EGFR-targeted therapy. In some embodiments, detection of the deletion of an MTAP gene, or of a portion thereof, and one or more mutations in an EGFR gene in the sample identifies the individual as one who may benefit from a treatment comprising an anti-cancer therapy, e.g., an anti-cancer therapy provided herein, such as an EGFR-targeted therapy. In some embodiments, responsive to knowledge of the deletion of an MTAP gene, or of a portion thereof, and one or more mutations in an EGFR gene in the sample, the individual is identified as one who may benefit from a treatment comprising an anti-cancer therapy, e.g., an anti-cancer therapy provided herein, such as an EGFR-targeted therapy. In some embodiments, the presence of the deletion of an MTAP gene, or of a portion thereof, and one or more mutations in a SMARCA4 gene in the sample identifies the individual as one who may benefit from a treatment comprising an anti-cancer therapy, e.g., an anti-cancer therapy provided herein, such as a SMARCA4-targeted therapy. In some embodiments, detection of the deletion of an MTAP gene, or of a portion thereof, and one or more mutations in a SMARCA4 gene in the sample identifies the individual as one who may benefit from a treatment comprising an anti-cancer therapy, e.g., an anti-cancer therapy provided herein, such as a SMARCA4-targeted therapy. In some embodiments, responsive to knowledge of the deletion of an MTAP gene, or of a portion thereof, and one or more mutations in a SMARCA4 gene in the sample, the individual is identified as one who may benefit from a treatment comprising an anti-cancer therapy, e.g., an anti-cancer therapy provided herein, such as a SMARCA4-targeted therapy. In some embodiments, the deletion of an MTAP gene, or of a portion thereof, and one or more mutations in an EGFR gene or in a SMARCA4 gene are detected using any suitable method known in the art or described herein. In some embodiments, the sample is a sample described herein. In some embodiments, the sample comprises cells from the cancer or is obtained from cells from the cancer, e.g., a NSCLC. In some embodiments, the presence of the deletion of an MTAP gene, or of a portion thereof, and one or more mutations in an EGFR gene or in a SMARCA4 gene in the sample identifies the individual as one who may benefit from a treatment comprising a PRMT5-targeted therapy and an additional cancer therapy, e.g., an EGFR-targeted therapy or a SMARCA4-targeted therapy. In some embodiments, detection of the deletion of an MTAP gene, or of a portion thereof, and one or more mutations in an EGFR gene or in a SMARCA4 gene in the sample identifies the individual as one who may benefit from a treatment comprising a PRMT5-targeted therapy and an additional cancer therapy, e.g., an EGFR-targeted therapy or a SMARCA4-targeted therapy. In some embodiments, responsive to knowledge of the deletion of an MTAP gene, or of a portion thereof, and one or more mutations in an EGFR gene or in a SMARCA4 gene in the sample, the individual is identified as one who may benefit from a treatment comprising a PRMT5-targeted therapy and an additional cancer therapy, e.g., an EGFR-targeted therapy or a SMARCA4-targeted therapy.

Also provided herein are methods of identifying or selecting a treatment, a therapy, or one or more treatment options for an individual having a cancer, such as a cancer described herein. In some embodiments, the cancer comprises a deletion of an MTAP gene, or of a portion thereof, and one or more mutations in an EGFR gene or in a SMARCA4 gene. In some embodiments, the methods comprise detecting the deletion of an MTAP gene, or of a portion thereof, and one or more mutations in an EGFR gene or in a SMARCA4 gene in a sample obtained from an individual having a cancer, such as a cancer described herein, e.g., a NSCLC. In some embodiments, the deletion of an MTAP gene, or of a portion thereof, and one or more mutations in an EGFR gene or in a SMARCA4 gene are detected using any suitable method known in the art or described herein. In some embodiments, detection of a deletion of an MTAP gene, or of a portion thereof, and one or more mutations in an EGFR gene or in a SMARCA4 gene in a sample obtained from an individual having a cancer, e.g., a cancer described herein, e.g., a NSCLC, identifies the individual as one who may benefit from an anti-cancer therapy, e.g., an anti-cancer therapy provided herein, such as an EGFR-targeted therapy or a SMARCA4-targeted therapy. In some embodiments, the presence of a deletion of an MTAP gene, or of a portion thereof, and one or more mutations in an EGFR gene or in a SMARCA4 gene in a sample obtained from an individual having a cancer, e.g., a cancer provided herein, e.g., a NSCLC, identifies the individual as one who may benefit from an anti-cancer therapy, such as an anti-cancer therapy provided herein, e.g., an EGFR-targeted therapy or a SMARCA4-targeted therapy. In some embodiments, responsive to detection of a deletion of an MTAP gene, or of a portion thereof, and one or more mutations in an EGFR gene or in a SMARCA4 gene in a sample obtained from an individual having a cancer, e.g., a cancer provided herein, e.g., a NSCLC, the individual is classified as a candidate to receive an anti-cancer therapy, such as an anti-cancer therapy provided herein, e.g., an EGFR-targeted therapy or a SMARCA4-targeted therapy. In some embodiments, responsive to detection of a deletion of an MTAP gene, or of a portion thereof, and one or more mutations in an EGFR gene or in a SMARCA4 gene in a sample obtained from an individual having a cancer, e.g., a cancer provided herein, e.g., a NSCLC, the individual is classified or identified as likely to respond to an anti-cancer therapy, such as an anti-cancer therapy provided herein, e.g., an EGFR-targeted therapy or a SMARCA4-targeted therapy. In some embodiments, the sample is a sample described herein. In some embodiments, detection of a deletion of an MTAP gene, or of a portion thereof, and one or more mutations in an EGFR gene in a sample obtained from an individual having a cancer, e.g., a cancer described herein, e.g., a NSCLC, identifies the individual as one who may benefit from an anti-cancer therapy, e.g., an anti-cancer therapy provided herein, such as an EGFR-targeted therapy. In some embodiments, the presence of a deletion of an MTAP gene, or of a portion thereof, and one or more mutations in an EGFR gene in a sample obtained from an individual having a cancer, e.g., a cancer provided herein, e.g., a NSCLC, identifies the individual as one who may benefit from an anti-cancer therapy, such as an anti-cancer therapy provided herein, e.g., an EGFR-targeted therapy. In some embodiments, responsive to detection of a deletion of an MTAP gene, or of a portion thereof, and one or more mutations in an EGFR gene in a sample obtained from an individual having a cancer, e.g., a cancer provided herein, e.g., a NSCLC, the individual is classified as a candidate to receive an anti-cancer therapy, such as an anti-cancer therapy provided herein, e.g., an EGFR-targeted therapy. In some embodiments, responsive to detection of a deletion of an MTAP gene, or of a portion thereof, and one or more mutations in an EGFR gene in a sample obtained from an individual having a cancer, e.g., a cancer provided herein, e.g., a NSCLC, the individual is classified or identified as likely to respond to an anti-cancer therapy, such as an anti-cancer therapy provided herein, e.g., an EGFR-targeted therapy. In some embodiments, detection of a deletion of an MTAP gene, or of a portion thereof, and one or more mutations in a SMARCA4 gene in a sample obtained from an individual having a cancer, e.g., a cancer described herein, e.g., a NSCLC, identifies the individual as one who may benefit from an anti-cancer therapy, e.g., an anti-cancer therapy provided herein, such as a SMARCA4-targeted therapy. In some embodiments, the presence of a deletion of an MTAP gene, or of a portion thereof, and one or more mutations in a SMARCA4 gene in a sample obtained from an individual having a cancer, e.g., a cancer provided herein, e.g., a NSCLC, identifies the individual as one who may benefit from an anti-cancer therapy, such as an anti-cancer therapy provided herein, e.g., a SMARCA4-targeted therapy. In some embodiments, responsive to detection of a deletion of an MTAP gene, or of a portion thereof, and one or more mutations in a SMARCA4 gene in a sample obtained from an individual having a cancer, e.g., a cancer provided herein, e.g., a NSCLC, the individual is classified as a candidate to receive an anti-cancer therapy, such as an anti-cancer therapy provided herein, e.g., a SMARCA4-targeted therapy. In some embodiments, responsive to detection of a deletion of an MTAP gene, or of a portion thereof, and one or more mutations in a SMARCA4 gene in a sample obtained from an individual having a cancer, e.g., a cancer provided herein, e.g., a NSCLC, the individual is classified or identified as likely to respond to an anti-cancer therapy, such as an anti-cancer therapy provided herein, e.g., a SMARCA4-targeted therapy. In some embodiments, the sample is a sample described herein. In some embodiments, the sample comprises cells from the cancer or is obtained from cells from the cancer, e.g., a NSCLC. In some embodiments, the methods further comprise generating a report, e.g., as described herein. In some embodiments, the report comprises a treatment, a therapy, or one or more treatment options identified or selected for the individual, e.g., based at least in part on detection of a deletion of an MTAP gene, or of a portion thereof, and one or more mutations in an EGFR gene or in a SMARCA4 gene in the sample. In some embodiments, detection of a deletion of an MTAP gene, or of a portion thereof, and one or more mutations in an EGFR gene or in a SMARCA4 gene in a sample obtained from an individual having a cancer, e.g., a cancer described herein, e.g., a NSCLC, identifies the individual as one who may benefit from a PRMT5-targeted therapy and an additional anti-cancer therapy, e.g., an EGFR-targeted therapy or a SMARCA4-targeted therapy. In some embodiments, the presence of a deletion of an MTAP gene, or of a portion thereof, and one or more mutations in an EGFR gene or in a SMARCA4 gene in a sample obtained from an individual having a cancer, e.g., a cancer provided herein, e.g., a NSCLC, identifies the individual as one who may benefit from a PRMT5-targeted therapy and an additional anti-cancer therapy, e.g., an EGFR-targeted therapy or a SMARCA4-targeted therapy. In some embodiments, responsive to detection of a deletion of an MTAP gene, or of a portion thereof, and one or more mutations in an EGFR gene or in a SMARCA4 gene in a sample obtained from an individual having a cancer, e.g., a cancer provided herein, e.g., a NSCLC, the individual is classified as a candidate to receive a PRMT5-targeted therapy and an additional anti-cancer therapy, e.g., an EGFR-targeted therapy or a SMARCA4-targeted therapy. In some embodiments, responsive to detection of a deletion of an MTAP gene, or of a portion thereof, and one or more mutations in an EGFR gene or in a SMARCA4 gene in a sample obtained from an individual having a cancer, e.g., a cancer provided herein, e.g., a NSCLC, the individual is classified or identified as likely to respond to a PRMT5-targeted therapy and an additional anti-cancer therapy, e.g., an EGFR-targeted therapy or a SMARCA4-targeted therapy.

In some aspects, provided herein are methods of identifying an individual having cancer, e.g., a NSCLC, who may benefit from an anti-cancer therapy, e.g., an anti-cancer therapy provided herein. In some embodiments, the methods comprise detecting a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, the methods comprise acquiring knowledge of the presence of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, the presence of the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the individual as one who may benefit from a treatment comprising an anti-cancer therapy, e.g., an anti-cancer therapy provided herein. In some embodiments, detection of the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the individual as one who may benefit from a treatment comprising an anti-cancer therapy, e.g., an anti-cancer therapy provided herein. In some embodiments, responsive to knowledge of the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is identified as one who may benefit from a treatment comprising an anti-cancer therapy, e.g., an anti-cancer therapy provided herein. In some embodiments, the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are detected using any suitable method known in the art or described herein. In some embodiments, the sample is a sample described herein. In some embodiments, the sample comprises cells from the cancer or is obtained from cells from the cancer, e.g., a NSCLC. In some embodiments, the presence of the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the individual as one who may benefit from a treatment comprising a PRMT5-targeted therapy and an additional cancer therapy, e.g., an anti-cancer therapy provided herein. In some embodiments, detection of the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the individual as one who may benefit from a treatment comprising a PRMT5-targeted therapy and an additional cancer therapy, e.g., an anti-cancer therapy provided herein. In some embodiments, responsive to knowledge of the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is identified as one who may benefit from a treatment comprising a PRMT5-targeted therapy and an additional cancer therapy, e.g., an anti-cancer therapy provided herein.

Also provided herein are methods of identifying or selecting a treatment, a therapy, or one or more treatment options for an individual having a cancer, such as a cancer described herein. In some embodiments, the cancer comprises a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the methods comprise detecting the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, such as a cancer described herein, e.g., a NSCLC. In some embodiments, the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are detected using any suitable method known in the art or described herein. In some embodiments, detection of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 ina sample obtained from an individual having a cancer, e.g., a cancer described herein, e.g., a NSCLC, identifies the individual as one who may benefit from an anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, identifies the individual as one who may benefit from an anti-cancer therapy, such as an anti-cancer therapy provided herein. In some embodiments, responsive to detection of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, the individual is classified as a candidate to receive an anti-cancer therapy, such as an anti-cancer therapy provided herein. In some embodiments, responsive to detection of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, the individual is classified or identified as likely to respond to an anti-cancer therapy, such as an anti-cancer therapy provided herein. In some embodiments, the sample is a sample described herein. In some embodiments, the sample comprises cells from the cancer or is obtained from cells from the cancer, e.g., a NSCLC. In some embodiments, the methods further comprise generating a report, e.g., as described herein. In some embodiments, the report comprises a treatment, a therapy, or one or more treatment options identified or selected for the individual, e.g., based at least in part on detection of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, detection of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer described herein, e.g., a NSCLC, identifies the individual as one who may benefit from a PRMT5-targeted therapy and an additional anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, identifies the individual as one who may benefit from a PRMT5-targeted therapy and an additional anti-cancer therapy, e.g., an anti-cancer therapy provided herein. In some embodiments, responsive to detection of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, the individual is classified as a candidate to receive a PRMT5-targeted therapy and an additional anti-cancer therapy, e.g., an anti-cancer therapy provided herein. In some embodiments, responsive to detection of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, the individual is classified or identified as likely to respond to a PRMT5-targeted therapy and an additional anti-cancer therapy, e.g., an anti-cancer therapy provided herein.

In some embodiments of any of the methods provided herein, the methods further comprise acquiring knowledge of or detecting in a sample from an individual a deletion of a CDKN2A gene, or of a portion thereof; and/or a deletion of a CDKN2B gene, or of a portion thereof. In some embodiments of any of the methods provided herein, the individual has a deletion of a CDKN2A gene, or of a portion thereof; and/or a deletion of a CDKN2B gene, or of a portion thereof. In some embodiments of any of the methods provided herein, the individual has a heterozygous or a homozygous deletion of a CDKN2A gene, or of a portion thereof; and/or a heterozygous or a homozygous deletion of a CDKN2B gene, or of a portion thereof. In some embodiments of any of the methods provided herein, the individual has a heterozygous or a homozygous deletion of the MTAP gene, or of a portion thereof.

### Methods of Diagnosing, Assessing, Screening, Monitoring or Predicting

In some aspects, provided herein are methods of diagnosing or assessing a deletion of an MTAP gene, or of a portion thereof, and one or more mutations in one or more genes selected from an EGFR gene or a SMARCA4 gene in a sample from an individual having a cancer, such as a NSCLC. In some embodiments, the methods comprise acquiring knowledge of the presence of a deletion of an MTAP gene, or of a portion thereof, and one or more mutations in an EGFR gene or in a SMARCA4 gene in a sample obtained from the individual. In some embodiments, the methods comprise detecting a deletion of an MTAP gene, or of a portion thereof, and one or more mutations in an EGFR gene or in a SMARCA4 gene in a sample obtained from the individual. In some embodiments, a deletion of an MTAP gene, or of a portion thereof, and one or more mutations in an EGFR gene or in a SMARCA4 gene are detected in a sample obtained from the individual using any method known in the art, such as one or more of the methods of detection of nucleic acid molecules or polypeptides described herein. In some embodiments, the methods further comprise providing a diagnosis or an assessment of the deletion of the MTAP gene, or of the portion thereof, and one or more mutations in an EGFR gene or in a SMARCA4 gene. In some embodiments, the diagnosis or assessment identifies the presence or absence of a deletion of an MTAP gene, or of a portion thereof, and one or more mutations in an EGFR gene or in a SMARCA4 gene in the sample. In some embodiments, the diagnosis or assessment identifies the cancer, such as a NSCLC provided herein, as likely to respond to an anti-cancer therapy, e.g., an anti-cancer therapy provided herein, such as an EGFR-targeted therapy and/or a SMARCA4-targeted therapy. In some embodiments, the presence of a deletion of an MTAP gene, or of a portion thereof, and one or more mutations in an EGFR gene or in a SMARCA4 gene in the sample identifies the cancer as likely to respond to an anti-cancer therapy, e.g., an anti-cancer therapy provided herein, such as an EGFR-targeted therapy or a SMARCA4-targeted therapy. In some embodiments, the diagnosis or assessment identifies the presence or absence of a deletion of an MTAP gene, or of a portion thereof, and one or more mutations in an EGFR gene in the sample. In some embodiments, the diagnosis or assessment identifies the cancer, such as a NSCLC provided herein, as likely to respond to an anti-cancer therapy, e.g., an anti-cancer therapy provided herein, such as an EGFR-targeted therapy. In some embodiments, the presence of a deletion of an MTAP gene, or of a portion thereof, and one or more mutations in an EGFR gene in the sample identifies the cancer as likely to respond to an anti-cancer therapy, e.g., an anti-cancer therapy provided herein, such as an EGFR-targeted therapy. In some embodiments, the diagnosis or assessment identifies the presence or absence of a deletion of an MTAP gene, or of a portion thereof, and one or more mutations in a SMARCA4 gene in the sample. In some embodiments, the diagnosis or assessment identifies the cancer, such as a NSCLC provided herein, as likely to respond to an anti-cancer therapy, e.g., an anti-cancer therapy provided herein, such as a SMARCA4-targeted therapy. In some embodiments, the presence of a deletion of an MTAP gene, or of a portion thereof, and one or more mutations in a SMARCA4 gene in the sample identifies the cancer as likely to respond to an anti-cancer therapy, e.g., an anti-cancer therapy provided herein, such as a SMARCA4-targeted therapy. In some embodiments, the sample is a sample described herein. In some embodiments, the sample comprises cells from the cancer or is obtained from cells from the cancer, e.g., from a NSCLC. In some embodiments, the individual has a cancer, is suspected of having a cancer, is being tested for a cancer, is being treated for a cancer, or is being tested for a susceptibility to a cancer, e.g., a NSCLC. In some embodiments, the diagnosis or assessment identifies the cancer, such as a NSCLC provided herein, as likely to respond to a PRMT5-targeted therapy and an additional anti-cancer therapy, e.g., an EGFR-targeted therapy or a SMARCA4-targeted therapy. In some embodiments, the presence of a deletion of an MTAP gene, or of a portion thereof, and one or more mutations in an EGFR gene or in a SMARCA4 gene in the sample identifies the cancer as likely to respond to a PRMT5-targeted therapy and an additional anti-cancer therapy, e.g., an EGFR-targeted therapy or a SMARCA4-targeted therapy.

In some aspects, provided herein are methods of diagnosing or assessing a cancer in an individual, e.g., a NSCLC. In some embodiments, the methods of diagnosing or assessing cancer comprise detecting a deletion of an MTAP gene, or of a portion thereof, and one or more mutations in an EGFR gene or in a SMARCA4 gene in a sample obtained from the individual, e.g., a sample comprising cells from the cancer, e.g., a NSCLC. In some embodiments, the methods comprise detecting a deletion of an MTAP gene, or of a portion thereof, and one or more mutations in an EGFR gene or in a SMARCA4 gene in a sample obtained from the individual using any method known in the art, such as one or more of the methods of detection of nucleic acid molecules or polypeptides described herein. In some embodiments, detection of a deletion of an MTAP gene, or of a portion thereof, and one or more mutations in an EGFR gene or in a SMARCA4 gene in a sample obtained from the individual identifies the cancer, e.g., the NSCLC, as likely to respond to an anti-cancer therapy, e.g., an anti-cancer therapy provided herein, such as an EGFR-targeted therapy or a SMARCA4-targeted therapy. In some embodiments, the presence of a deletion of an MTAP gene, or of a portion thereof, and one or more mutations in an EGFR gene or in a SMARCA4 gene in a sample obtained from the individual identifies the cancer, e.g., the NSCLC, as likely to respond to an anti-cancer therapy, e.g., an anti-cancer therapy provided herein, such as an EGFR-targeted therapy or a SMARCA4-targeted therapy. In some embodiments, the methods further comprise providing a diagnosis or an assessment of the cancer, e.g., the NSCLC, or of the deletion of the MTAP gene, or of a portion thereof, and one or more mutations in an EGFR gene or in a SMARCA4 gene. In some embodiments, the diagnosis or assessment identifies the cancer as likely to respond to an anti-cancer therapy, e.g., an anti-cancer therapy provided herein, such as an EGFR-targeted therapy or a SMARCA4-targeted therapy. In some embodiments, detection of a deletion of an MTAP gene, or of a portion thereof, and one or more mutations in an EGFR gene in a sample obtained from the individual identifies the cancer, e.g., the NSCLC, as likely to respond to an anti-cancer therapy, e.g., an anti-cancer therapy provided herein, such as an EGFR-targeted therapy. In some embodiments, the presence of a deletion of an MTAP gene, or of a portion thereof, and one or more mutations in an EGFR gene in a sample obtained from the individual identifies the cancer, e.g., the NSCLC, as likely to respond to an anti-cancer therapy, e.g., an anti-cancer therapy provided herein, such as an EGFR-targeted therapy. In some embodiments, the methods further comprise providing a diagnosis or an assessment of the cancer, e.g., the NSCLC, or of the deletion of the MTAP gene, or of a portion thereof, and one or more mutations in an EGFR gene. In some embodiments, the diagnosis or assessment identifies the cancer as likely to respond to an anti-cancer therapy, e.g., an anti-cancer therapy provided herein, such as an EGFR-targeted therapy. In some embodiments, detection of a deletion of an MTAP gene, or of a portion thereof, and one or more mutations in a SMARCA4 gene in a sample obtained from the individual identifies the cancer, e.g., the NSCLC, as likely to respond to an anti-cancer therapy, e.g., an anti-cancer therapy provided herein, such as a SMARCA4-targeted therapy. In some embodiments, the presence of a deletion of an MTAP gene, or of a portion thereof, and one or more mutations in a SMARCA4 gene in a sample obtained from the individual identifies the cancer, e.g., the NSCLC, as likely to respond to an anti-cancer therapy, e.g., an anti-cancer therapy provided herein, such as a SMARCA4-targeted therapy. In some embodiments, the methods further comprise providing a diagnosis or an assessment of the cancer, e.g., the NSCLC, or of the deletion of the MTAP gene, or of a portion thereof, and one or more mutations in a SMARCA4 gene. In some embodiments, the diagnosis or assessment identifies the cancer as likely to respond to an anti-cancer therapy, e.g., an anti-cancer therapy provided herein, such as a SMARCA4-targeted therapy. In some embodiments, the diagnosis or assessment identifies the presence or absence of the deletion of the MTAP gene, or of a portion thereof, and one or more mutations in an EGFR gene or in a SMARCA4 gene in the sample. In some embodiments, detection of a deletion of an MTAP gene, or of a portion thereof, and one or more mutations in an EGFR gene or in a SMARCA4 gene in a sample obtained from the individual identifies the cancer, e.g., the NSCLC, as likely to respond to a PRMT5-targeted therapy and an additional anti-cancer therapy, e.g., an EGFR-targeted therapy or a SMARCA4-targeted therapy. In some embodiments, the presence of a deletion of an MTAP gene, or of a portion thereof, and one or more mutations in an EGFR gene or in a SMARCA4 gene in a sample obtained from the individual identifies the cancer, e.g., the NSCLC, as likely to respond to a PRMT5-targeted therapy and an additional anti-cancer therapy, e.g., an EGFR-targeted therapy or a SMARCA4-targeted therapy. In some embodiments, the diagnosis or assessment identifies the cancer as likely to respond to a PRMT5-targeted therapy and an additional anti-cancer therapy, e.g., an EGFR-targeted therapy or a SMARCA4-targeted therapy.

In some aspects, provided herein are methods of predicting survival of an individual having a cancer, e.g., a cancer provided herein, e.g., a NSCLC. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in an EGFR gene or in a SMARCA4 gene in a sample from the individual. In some embodiments, the methods comprise detecting a deletion of an MTAP gene, or of a portion thereof, and one or more mutations in an EGFR gene or in a SMARCA4 gene in a sample from the individual. In some embodiments, responsive to acquiring knowledge of a deletion of an MTAP gene, or of a portion thereof, and one or more mutations in an EGFR gene or in a SMARCA4 gene in the sample, the individual is predicted to have longer survival after treatment with an anti-cancer therapy, e.g., an anti-cancer therapy provided herein (e.g., an EGFR-targeted therapy or a SMARCA4-targeted therapy), for example, as compared to an individual whose cancer does not exhibit the deletion of the MTAP gene, or of a portion thereof, and one or more mutations in an EGFR gene or in a SMARCA4 gene. In some embodiments, responsive to detecting a deletion of an MTAP gene, or of a portion thereof, and one or more mutations in an EGFR gene or in a SMARCA4 gene in the sample, the individual is predicted to have longer survival after treatment with an anti-cancer therapy, e.g., an anti-cancer therapy provided herein (e.g., an EGFR-targeted therapy or a SMARCA4-targeted therapy), for example, as compared to an individual whose cancer does not exhibit the deletion of the MTAP gene, or of a portion thereof, and one or more mutations in an EGFR gene or in a SMARCA4 gene. In some embodiments, responsive to acquiring knowledge of a deletion of an MTAP gene, or of a portion thereof, and one or more mutations in an EGFR gene in the sample, the individual is predicted to have longer survival after treatment with an anti-cancer therapy, e.g., an anti-cancer therapy provided herein (e.g., an EGFR-targeted therapy), for example, as compared to an individual whose cancer does not exhibit the deletion of the MTAP gene, or of a portion thereof, and one or more mutations in an EGFR gene. In some embodiments, responsive to detecting a deletion of an MTAP gene, or of a portion thereof, and one or more mutations in an EGFR gene in the sample, the individual is predicted to have longer survival after treatment with an anti-cancer therapy, e.g., an anti-cancer therapy provided herein (e.g., an EGFR-targeted therapy), for example, as compared to an individual whose cancer does not exhibit the deletion of the MTAP gene, or of a portion thereof, and one or more mutations in an EGFR gene. In some embodiments, responsive to acquiring knowledge of a deletion of an MTAP gene, or of a portion thereof, and one or more mutations in a SMARCA4 gene in the sample, the individual is predicted to have longer survival after treatment with an anti-cancer therapy, e.g., an anti-cancer therapy provided herein (e.g., a SMARCA4-targeted therapy), for example, as compared to an individual whose cancer does not exhibit the deletion of the MTAP gene, or of a portion thereof, and one or more mutations in a SMARCA4 gene. In some embodiments, responsive to detecting a deletion of an MTAP gene, or of a portion thereof, and one or more mutations in a SMARCA4 gene in the sample, the individual is predicted to have longer survival after treatment with an anti-cancer therapy, e.g., an anti-cancer therapy provided herein (e.g., a SMARCA4-targeted therapy), for example, as compared to an individual whose cancer does not exhibit the deletion of the MTAP gene, or of a portion thereof, and one or more mutations in a SMARCA4 gene. In some embodiments, the methods further comprise providing a diagnosis or an assessment. In some embodiments, the diagnosis or assessment identifies the presence or absence of the deletion of an MTAP gene, or of a portion thereof, and one or more mutations in an EGFR gene or in a SMARCA4 gene in the sample. In some embodiments, the diagnosis or assessment identifies the individual as being predicted to have longer survival after treatment with an anti-cancer therapy, e.g., an anti-cancer therapy provided herein (e.g., an EGFR-targeted therapy or a SMARCA4-targeted therapy), for example, as compared to an individual whose cancer does not exhibit the deletion of the MTAP gene, or of a portion thereof, and one or more mutations in an EGFR gene or in a SMARCA4 gene. In some embodiments, the diagnosis or assessment identifies the individual as being predicted to have longer survival after treatment with an anti-cancer therapy, e.g., an anti-cancer therapy provided herein (e.g., an EGFR-targeted therapy), for example, as compared to an individual whose cancer does not exhibit the deletion of the MTAP gene, or of a portion thereof, and one or more mutations in an EGFR gene. In some embodiments, the diagnosis or assessment identifies the individual as being predicted to have longer survival after treatment with an anti-cancer therapy, e.g., an anti-cancer therapy provided herein (e.g., a SMARCA4-targeted therapy), for example, as compared to an individual whose cancer does not exhibit the deletion of the MTAP gene, or of a portion thereof, and one or more mutations in a SMARCA4 gene. In some embodiments, the sample is a sample described herein. In some embodiments, the sample comprises cells from the cancer or is obtained from cells from the cancer, e.g., the NSCLC. In some embodiments, responsive to acquiring knowledge of a deletion of an MTAP gene, or of a portion thereof, and one or more mutations in an EGFR gene or in a SMARCA4 gene in the sample, the individual is predicted to have longer survival after treatment with a PRMT5-targeted therapy and an additional anti-cancer therapy, e.g., an EGFR-targeted therapy or a SMARCA4-targeted therapy, for example, as compared to an individual whose cancer does not exhibit the deletion of the MTAP gene, or of a portion thereof, and one or more mutations in an EGFR gene or in a SMARCA4 gene. In some embodiments, responsive to detecting a deletion of an MTAP gene, or of a portion thereof, and one or more mutations in an EGFR gene or in a SMARCA4 gene in the sample, the individual is predicted to have longer survival after treatment with a PRMT5-targeted therapy and an additional anti-cancer therapy, e.g., an EGFR-targeted therapy or a SMARCA4-targeted therapy, for example, as compared to an individual whose cancer does not exhibit the deletion of the MTAP gene, or of a portion thereof, and one or more mutations in an EGFR gene or in a SMARCA4 gene. In some embodiments, the diagnosis or assessment identifies the individual as being predicted to have longer survival after treatment with a PRMT5-targeted therapy and an additional anti-cancer therapy, e.g., an EGFR-targeted therapy or a SMARCA4-targeted therapy, for example, as compared to an individual whose cancer does not exhibit the deletion of the MTAP gene, or of a portion thereof, and one or more mutations in an EGFR gene or in a SMARCA4 gene.

In some aspects, provided herein are methods of screening an individual having cancer, suspected of having cancer, being tested for cancer, being treated for cancer, or being tested for a susceptibility to cancer, e.g., a NSCLC. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in an EGFR gene or in a SMARCA4 gene in a sample from the individual. In some embodiments, the methods comprise detecting a deletion of an MTAP gene, or of a portion thereof, and one or more mutations in an EGFR gene or in a SMARCA4 gene in a sample from the individual. In some embodiments, responsive to acquiring knowledge of a deletion of an MTAP gene, or of a portion thereof, and one or more mutations in an EGFR gene or in a SMARCA4 gene in the sample, the individual is predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in an EGFR gene or in a SMARCA4 gene. In some embodiments, responsive to detecting a deletion of an MTAP gene, or of a portion thereof, and one or more mutations in an EGFR gene or in a SMARCA4 gene in the sample, the individual is predicted to have increased risk of cancer recurrence, aggressive cancer, anti-cancer therapy resistance, or poor prognosis, for example, as compared to an individual whose cancer does not exhibit the deletion of an MTAP gene, or of a portion thereof, and one or more mutations in an EGFR gene or in a SMARCA4 gene. In some embodiments, the methods further comprise providing a diagnosis or an assessment. In some embodiments, the diagnosis or assessment identifies the presence or absence of the deletion of the MTAP gene, or of a portion thereof, and one or more mutations in an EGFR gene or in a SMARCA4 gene in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in an EGFR gene or in a SMARCA4 gene. In some embodiments, the sample is a sample described herein. In some embodiments, the sample comprises cells from the cancer or is obtained from cells from the cancer, e.g., the NSCLC.

In some embodiments, the methods further comprise selectively enriching for one or more nucleic acids comprising nucleotide sequences that comprise one or more mutations in an EGFR gene or in a SMARCA4 gene to produce an enriched sample, e.g., using a reagent known in the art or provided herein, such as a bait, probe, or oligonucleotide described herein. In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample. In some embodiments, the methods further comprise selectively enriching for one or more polypeptides comprising amino acid sequences that comprise one or more mutations in an EGFR gene or in a SMARCA4 gene to produce an enriched sample, e.g., using a reagent known in the art or provided herein, such as an antibody described herein. In some embodiments, the methods further comprise selectively enriching for one or more nucleic acids comprising nucleotide sequences that comprise a deletion of an MTAP gene, or of a portion thereof, to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise a deletion of an MTAP gene, or of a portion thereof, to produce an enriched sample, e.g., using a reagent known in the art or provided herein, such as an antibody described herein.

In some aspects, provided herein are methods of diagnosing or assessing a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from an individual having a cancer, such as a NSCLC. In some embodiments, the methods comprise acquiring knowledge of the presence of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, the methods comprise detecting a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are detected in a sample obtained from the individual using any method known in the art, such as one or more of the methods of detection of nucleic acid molecules or polypeptides described herein. In some embodiments, the methods further comprise providing a diagnosis or an assessment of the deletion of the MTAP gene, or of the portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the diagnosis or assessment identifies the presence or absence of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, the diagnosis or assessment identifies the cancer, such as a NSCLC provided herein, as likely to respond to an anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the cancer as likely to respond to an anti-cancer therapy, e.g., an anti-cancer therapy provided herein. In some embodiments, the sample is a sample described herein. In some embodiments, the sample comprises cells from the cancer or is obtained from cells from the cancer, e.g., from a NSCLC. In some embodiments, the individual has a cancer, is suspected of having a cancer, is being tested for a cancer, is being treated for a cancer, or is being tested for a susceptibility to a cancer, e.g., a NSCLC. In some embodiments, the diagnosis or assessment identifies the cancer, such as a NSCLC provided herein, as likely to respond to a PRMT5-targeted therapy and an additional anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the cancer as likely to respond to a PRMT5-targeted therapy and an additional anti-cancer therapy, e.g., an anti-cancer therapy provided herein.

In some aspects, provided herein are methods of diagnosing or assessing a cancer in an individual, e.g., a NSCLC. In some embodiments, the methods of diagnosing or assessing cancer comprise detecting a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual, e.g., a sample comprising cells from the cancer, e.g., a NSCLC. In some embodiments, the methods comprise detecting a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual using any method known in the art, such as one or more of the methods of detection of nucleic acid molecules or polypeptides described herein. In some embodiments, detection of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 ina sample obtained from the individual identifies the cancer, e.g., the NSCLC, as likely to respond to an anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual identifies the cancer, e.g., the NSCLC, as likely to respond to an anti-cancer therapy, e.g., an anti-cancer therapy provided herein. In some embodiments, the methods further comprise providing a diagnosis or an assessment of the cancer, e.g., the NSCLC, or of the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the diagnosis or assessment identifies the cancer as likely to respond to an anti-cancer therapy, e.g., an anti-cancer therapy provided herein. In some embodiments, the diagnosis or assessment identifies the presence or absence of the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, detection of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, orRB ina sample obtained from the individual identifies the cancer, e.g., the NSCLC, as likely to respond to a PRMT5-targeted therapy and an additional anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual identifies the cancer, e.g., the NSCLC, as likely to respond to a PRMT5-targeted therapy and an additional anti-cancer therapy, e.g., an anti-cancer therapy provided herein. In some embodiments, the diagnosis or assessment identifies the cancer as likely to respond to a PRMT5-targeted therapy and an additional anti-cancer therapy, e.g., an anti-cancer therapy provided herein.

In some aspects, provided herein are methods of predicting survival of an individual having a cancer, e.g., a cancer provided herein, e.g., a NSCLC. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from the individual. In some embodiments, the methods comprise detecting a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from the individual. In some embodiments, responsive to acquiring knowledge of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have longer survival after treatment with an anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, responsive to detecting a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have longer survival after treatment with an anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the methods further comprise providing a diagnosis or an assessment. In some embodiments, the diagnosis or assessment identifies the presence or absence of the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the sample is a sample described herein. In some embodiments, the sample comprises cells from the cancer or is obtained from cells from the cancer, e.g., the NSCLC. In some embodiments, responsive to acquiring knowledge of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have longer survival after treatment with a PRMT5-targeted therapy and an additional anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, responsive to detecting a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have longer survival after treatment with a PRMT5-targeted therapy and an additional anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the diagnosis or assessment identifies the individual as being predicted to have longer survival after treatment with a PRMT5-targeted therapy and an additional anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1.

In some aspects, provided herein are methods of screening an individual having cancer, suspected of having cancer, being tested for cancer, being treated for cancer, or being tested for a susceptibility to cancer, e.g., a NSCLC. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from the individual. In some embodiments, the methods comprise detecting a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from the individual. In some embodiments, responsive to acquiring knowledge of a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, responsive to detecting a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have increased risk of cancer recurrence, aggressive cancer, anti-cancer therapy resistance, or poor prognosis, for example, as compared to an individual whose cancer does not exhibit the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the methods further comprise providing a diagnosis or an assessment. In some embodiments, the diagnosis or assessment identifies the presence or absence of the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the sample is a sample described herein. In some embodiments, the sample comprises cells from the cancer or is obtained from cells from the cancer, e.g., the NSCLC.

In some embodiments, the methods further comprise selectively enriching for one or more nucleic acids comprising nucleotide sequences that comprise one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 to produce an enriched sample, e.g., using a reagent known in the art or provided herein, such as a bait, probe, or oligonucleotide described herein. In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample. In some embodiments, the methods further comprise selectively enriching for one or more polypeptides comprising amino acid sequences that comprise one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 to produce an enriched sample, e.g., using a reagent known in the art or provided herein, such as an antibody described herein. In some embodiments, the methods further comprise selectively enriching for one or more nucleic acids comprising nucleotide sequences that comprise a deletion of an MTAP gene, or of a portion thereof, to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise a deletion of an MTAP gene, or of a portion thereof, to produce an enriched sample, e.g., using a reagent known in the art or provided herein, such as an antibody described herein.

In some embodiments of any of the methods provided herein, the methods further comprise acquiring knowledge of or detecting in a sample from an individual a deletion of a CDKN2A gene, or of a portion thereof; and/or a deletion of a CDKN2B gene, or of a portion thereof. In some embodiments of any of the methods provided herein, the individual has a deletion of a CDKN2A gene, or of a portion thereof; and/or a deletion of a CDKN2B gene, or of a portion thereof. In some embodiments of any of the methods provided herein, the individual has a heterozygous or a homozygous deletion of a CDKN2A gene, or of a portion thereof; and/or a heterozygous or a homozygous deletion of a CDKN2B gene, or of a portion thereof. In some embodiments of any of the methods provided herein, the individual has a heterozygous or a homozygous deletion of the MTAP gene, or of a portion thereof.

Methods of Treatment

Also provided herein are methods of treating or delaying progression of a cancer in an individual, such as a cancer provided herein, e.g., a NSCLC. In some embodiments, the individual has a cancer comprising a deletion of an MTAP gene, or of a portion thereof, and one or more mutations in an EGFR gene or in a SMARCA4 gene. In some embodiments, the methods of treating or delaying progression of a cancer of the disclosure, e.g., a NSCLC comprising a deletion of an MTAP gene, or of a portion thereof, and one or more mutations in an EGFR gene or in a SMARCA4 gene, comprise administering to the individual a therapeutically effective amount of an anti-cancer therapy, such as an anti-cancer therapy provided herein, e.g., an EGFR-targeted therapy or a SMARCA4-targeted therapy. In some embodiments, the methods of treating or delaying progression of a cancer of the disclosure, e.g., a NSCLC comprising a deletion of an MTAP gene, or of a portion thereof, and one or more mutations in an EGFR gene or in a SMARCA4 gene, comprise administering to the individual an effective amount of an anti-cancer therapy, such as an anti-cancer therapy provided herein (e.g., an EGFR-targeted therapy or a SMARCA4-targeted therapy), responsive to knowledge of the presence of the deletion of an MTAP gene, or of a portion thereof, and one or more mutations in an EGFR gene or in a SMARCA4 gene in a sample obtained from the individual. In some embodiments, the sample is a sample described herein. In some embodiments, the sample comprises cells from the cancer or is obtained from cells from the cancer. The methods of treatment disclosed herein may include any of the anti-cancer therapies and/or therapeutic agents described herein, e.g., infra.

In some embodiments, the individual has a cancer comprising a deletion of an MTAP gene, or of a portion thereof, and one or more mutations in an EGFR gene. In some embodiments, the methods of treating or delaying progression of a cancer of the disclosure, e.g., a NSCLC comprising a deletion of an MTAP gene, or of a portion thereof, and one or more mutations in an EGFR gene, comprise administering to the individual a therapeutically effective amount of an anti-cancer therapy, such as an anti-cancer therapy provided herein, e.g., an EGFR-targeted therapy. In some embodiments, the methods of treating or delaying progression of a cancer of the disclosure, e.g., a NSCLC comprising a deletion of an MTAP gene, or of a portion thereof, and one or more mutations in an EGFR gene, comprise administering to the individual an effective amount of an anti-cancer therapy, such as an anti-cancer therapy provided herein (e.g., an EGFR-targeted therapy), responsive to knowledge of the presence of the deletion of an MTAP gene, or of a portion thereof, and one or more mutations in an EGFR gene in a sample obtained from the individual. In some embodiments, the EGFR-targeted therapy comprises one or more of an EGFR inhibitor, an HSP90 inhibitor, a VEGFR/EGFR dual inhibitor, a MEK inhibitor, or a Raf inhibitor.

In some embodiments, the individual has a cancer comprising a deletion of an MTAP gene, or of a portion thereof, and one or more mutations in a SMARCA4 gene. In some embodiments, the methods of treating or delaying progression of a cancer of the disclosure, e.g., a NSCLC comprising a deletion of an MTAP gene, or of a portion thereof, and one or more mutations in a SMARCA4 gene, comprise administering to the individual a therapeutically effective amount of an anti-cancer therapy, such as an anti-cancer therapy provided herein, e.g., a SMARCA4-targeted therapy. In some embodiments, the methods of treating or delaying progression of a cancer of the disclosure, e.g., a NSCLC comprising a deletion of an MTAP gene, or of a portion thereof, and one or more mutations in a SMARCA4 gene, comprise administering to the individual an effective amount of an anti-cancer therapy, such as an anti-cancer therapy provided herein (e.g., a SMARCA4-targeted therapy), responsive to knowledge of the presence of the deletion of an MTAP gene, or of a portion thereof, and one or more mutations in a SMARCA4 gene in a sample obtained from the individual. In some embodiments, the SMARCA4-targeted therapy comprises one or more of a CDK4/6 inhibitor, an Aurora kinase (AURK) inhibitor, an ATR inhibitor, an EZH2 inhibitor, a KDM6 inhibitor, a kinase inhibitor, a cisplatin-based chemotherapy, or an immune checkpoint inhibitor.

Also provided herein are methods of treating or delaying progression of a cancer in an individual, such as a cancer provided herein, e.g., a NSCLC. In some embodiments, the individual has a cancer comprising a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the methods of treating or delaying progression of a cancer of the disclosure, e.g., a NSCLC comprising a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprise administering to the individual a therapeutically effective amount of an anti-cancer therapy, such as an anti-cancer therapy provided herein. In some embodiments, the methods of treating or delaying progression of a cancer of the disclosure, e.g., a NSCLC comprising a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprise administering to the individual an effective amount of an anti-cancer therapy, such as an anti-cancer therapy provided herein, responsive to knowledge of the presence of the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, the sample is a sample described herein. In some embodiments, the sample comprises cells from the cancer or is obtained from cells from the cancer. The methods of treatment disclosed herein may include any of the anti-cancer therapies and/or therapeutic agents described herein, e.g., infra.

In some embodiments, the methods of treating or delaying progression of a cancer of the disclosure, e.g., a NSCLC comprising a deletion of an MTAP gene, or of a portion thereof, and one or more mutations in an EGFR gene or in a SMARCA4 gene, comprise administering to the individual a therapeutically effective amount of a PRMT5-targeted therapy and an additional anti-cancer therapy, e.g., an EGFR-targeted therapy or a SMARCA4-targeted therapy. In some embodiments, the methods of treating or delaying progression of a cancer of the disclosure, e.g., a NSCLC comprising a deletion of an MTAP gene, or of a portion thereof, and one or more mutations in an EGFR gene or in a SMARCA4 gene, comprise administering to the individual an effective amount of a PRMT5-targeted therapy and an additional anti-cancer therapy, e.g., an EGFR-targeted therapy or a SMARCA4-targeted therapy, responsive to knowledge of the presence of the deletion of an MTAP gene, or of a portion thereof, and one or more mutations in an EGFR gene or in a SMARCA4 gene in a sample obtained from the individual.

In some embodiments, the methods of treating or delaying progression of a cancer of the disclosure, e.g., a NSCLC comprising a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprise administering to the individual a therapeutically effective amount of a PRMT5-targeted therapy and an additional anti-cancer therapy, e.g., an anti-cancer therapy provided herein. In some embodiments, the methods of treating or delaying progression of a cancer of the disclosure, e.g., a NSCLC comprising a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprise administering to the individual an effective amount of a PRMT5-targeted therapy and an additional anti-cancer therapy, e.g., an anti-cancer therapy provided herein, responsive to knowledge of the presence of the deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual.

In some embodiments, the anti-cancer therapy is administered in combination with a second anti-cancer therapy or agent, such as any of the anti-cancer agents or therapies described herein. In some embodiments, the second anti-cancer therapy or agent comprises a PRMT5-targeted therapy. In some embodiments, the anti-cancer therapy comprises any of the anti-cancer therapies provided herein and a PRMT5-targeted therapy. In some embodiments, the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

In some embodiments of any of the methods provided herein, the methods further comprise acquiring knowledge of or detecting in a sample from an individual a deletion of a CDKN2A gene, or of a portion thereof; and/or a deletion of a CDKN2B gene, or of a portion thereof. In some embodiments of any of the methods provided herein, the individual has a deletion of a CDKN2A gene, or of a portion thereof; and/or a deletion of a CDKN2B gene, or of a portion thereof. In some embodiments of any of the methods provided herein, the individual has a heterozygous or a homozygous deletion of a CDKN2A gene, or of a portion thereof; and/or a heterozygous or a homozygous deletion of a CDKN2B gene, or of a portion thereof. In some embodiments of any of the methods provided herein, the individual has a heterozygous or a homozygous deletion of the MTAP gene, or of a portion thereof.

Reporting

In some embodiments, the methods provided herein comprise generating a report, and/or providing a report to party.

In some embodiments, a report according to the present disclosure comprises information about one or more of: a deletion of an MTAP gene, or of a portion thereof, and one or more mutations in an EGFR gene or in a SMARCA4 gene; a cancer of the disclosure (e.g., a NSCLC), e.g., comprising a deletion of an MTAP gene, or of a portion thereof, and one or more mutations in an EGFR gene or in a SMARCA4 gene; or a treatment, a therapy, or one or more treatment options (e.g., an EGFR-targeted therapy or a SMARCA4-targeted therapy) for an individual having a cancer, such as a cancer of the disclosure (e.g., a NSCLC, e.g., comprising a deletion of an MTAP gene, or of a portion thereof, and one or more mutations in an EGFR gene or in a SMARCA4 gene). In some embodiments, a report according to the present disclosure comprises information about one or more of: a deletion of an MTAP gene, or of a portion thereof, and one or more mutations in an EGFR gene; a cancer of the disclosure (e.g., a NSCLC), e.g., comprising a deletion of an MTAP gene, or of a portion thereof, and one or more mutations in an EGFR gene; or a treatment, a therapy, or one or more treatment options (e.g., an EGFR-targeted therapy) for an individual having a cancer, such as a cancer of the disclosure (e.g., a NSCLC, e.g., comprising a deletion of an MTAP gene, or of a portion thereof, and one or more mutations in an EGFR gene). In some embodiments, a report according to the present disclosure comprises information about one or more of: a deletion of an MTAP gene, or of a portion thereof, and one or more mutations in a SMARCA4 gene; a cancer of the disclosure (e.g., a NSCLC), e.g., comprising a deletion of an MTAP gene, or of a portion thereof, and one or more mutations in a SMARCA4 gene; or a treatment, a therapy, or one or more treatment options (e.g., a SMARCA4-targeted therapy) for an individual having a cancer, such as a cancer of the disclosure (e.g., a NSCLC, e.g., comprising a deletion of an MTAP gene, or of a portion thereof, and one or more mutations in a SMARCA4 gene). In some embodiments, the report further comprises information about one or more of: a deletion of a CDKN2A gene, or of a portion thereof; and/or a deletion of a CDKN2B gene, or of a portion thereof, e.g., a heterozygous or a homozygous deletion of a CDKN2A gene, or of a portion thereof; and/or a heterozygous or a homozygous deletion of a CDKN2B gene, or of a portion thereof. In some embodiments, the report comprises information about the presence or absence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1.

In some embodiments, a report according to the present disclosure comprises information about the presence or absence of a deletion of an MTAP gene, or of a portion thereof, and one or more mutations in an EGFR gene or in a SMARCA4 gene in a sample obtained from an individual, such as an individual having a cancer, e.g., a cancer provided herein, e.g., a NSCLC. In one embodiment, a report according to the present disclosure indicates that a deletion of an MTAP gene, or of a portion thereof, and one or more mutations in an EGFR gene or in a SMARCA4 gene are present in a sample obtained from the individual. In one embodiment, a report according to the present disclosure indicates that a deletion of an MTAP gene, or of a portion thereof, and one or more mutations in an EGFR gene or in a SMARCA4 gene are not present in a sample obtained from the individual. In one embodiment, a report according to the present disclosure indicates that a deletion of an MTAP gene, or of a portion thereof, and one or more mutations in an EGFR gene or in a SMARCA4 gene have been detected in a sample obtained from the individual. In one embodiment, a report according to the present disclosure indicates that a deletion of an MTAP gene, or of a portion thereof, and one or more mutations in an EGFR gene or in a SMARCA4 gene have not been detected in a sample obtained from the individual. In some embodiments, the report comprises an identifier for the individual from which the sample was obtained. In some embodiments, a report according to the present disclosure comprises information about the presence or absence of a deletion of a CDKN2A gene, or of a portion thereof, and/or a deletion of a CDKN2B gene, or of a portion thereof, in a sample obtained from an individual, such as an individual having a cancer, e.g., a cancer provided herein, e.g., a NSCLC. In some embodiments, a report according to the present disclosure comprises information about the presence or absence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual, such as an individual having a cancer, e.g., a cancer provided herein, e.g., a NSCLC. In one embodiment, a report according to the present disclosure indicates that a deletion of a CDKN2A gene, or of a portion thereof, and/or a deletion of a CDKN2B gene, or of a portion thereof, are present in a sample obtained from the individual. In one embodiment, a report according to the present disclosure indicates that one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are present in a sample obtained from the individual. In one embodiment, a report according to the present disclosure indicates that a deletion of a CDKN2A gene, or of a portion thereof, and/or a deletion of a CDKN2B gene, or of a portion thereof, are not present in a sample obtained from the individual. In one embodiment, a report according to the present disclosure indicates that one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are not present in a sample obtained from the individual. In one embodiment, a report according to the present disclosure indicates that a deletion of a CDKN2A gene, or of a portion thereof, and/or a deletion of a CDKN2B gene, or of a portion thereof, have been detected in a sample obtained from the individual. In one embodiment, a report according to the present disclosure indicates that one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 have been detected in a sample obtained from the individual. In one embodiment, a report according to the present disclosure indicates that a deletion of a CDKN2A gene, or of a portion thereof, and/or a deletion of a CDKN2B gene, or of a portion thereof, have not been detected in a sample obtained from the individual. In one embodiment, a report according to the present disclosure indicates that one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 have not been detected in a sample obtained from the individual. In some embodiments, the report comprises an identifier for the individual from which the sample was obtained.

In some embodiments, the report includes information on the role of a deletion of an MTAP gene, or of a portion thereof, and one or more mutations in an EGFR gene or in a SMARCA4 gene, or of the corresponding wild type counterparts, in disease, such as in cancer, e.g., a NSCLC. Such information can include one or more of: information on prognosis of a cancer, such as a cancer provided herein, e.g., comprising a deletion of an MTAP gene, or of a portion thereof, and one or more mutations in an EGFR gene or in a SMARCA4 gene; information on resistance of a cancer, such as a cancer provided herein, e.g., a NSCLC, such as a NSCLC comprising a deletion of an MTAP gene, or of a portion thereof, and one or more mutations in an EGFR gene or in a SMARCA4 gene, to one or more treatments; information on potential or suggested therapeutic options (e.g., such as an anti-cancer therapy provided herein, or a treatment selected or identified according to the methods provided herein, e.g., an EGFR-targeted therapy or a SMARCA4-targeted therapy); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein, e.g., an EGFR-targeted therapy or SMARCA4-targeted therapy) to an individual having a cancer, such as a cancer provided herein (e.g., a NSCLC), e.g., comprising a deletion of an MTAP gene, or of a portion thereof, and one or more mutations in an EGFR gene or in a SMARCA4 gene, and identified in the report. In some embodiments, the report includes information or a recommendation on the administration of a treatment (e.g., an anti-cancer therapy provided herein, or a treatment selected or identified according to the methods provided herein, such as an EGFR-targeted therapy or a SMARCA4-targeted therapy). In some embodiments, the report includes information on the role of a deletion of a CDKN2A gene, or of a portion thereof, and/or a deletion of a CDKN2B gene, or of a portion thereof, or of the corresponding wild type counterparts, in disease, such as in cancer, e.g., a NSCLC. In some embodiments, the report includes information on the role of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, or of the corresponding wild type counterparts, in disease, such as in cancer, e.g., a NSCLC. Such information can include one or more of: information on prognosis of a cancer, such as a cancer provided herein, e.g., comprising a deletion of a CDKN2A gene, or of a portion thereof, and/or a deletion of a CDKN2B gene, or of a portion thereof; information on resistance of a cancer, such as a cancer provided herein, e.g., a NSCLC, such as a NSCLC comprising a deletion of a CDKN2A gene, or of a portion thereof, and/or a deletion of a CDKN2B gene, or of a portion thereof, to one or more treatments; information on potential or suggested therapeutic options (e.g., such as an anti-cancer therapy provided herein, or a treatment selected or identified according to the methods provided herein); or information on therapeutic options that should be avoided. Such information can include one or more of: information on prognosis of a cancer, such as a cancer provided herein, e.g., comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1; information on resistance of a cancer, such as a cancer provided herein, e.g., a NSCLC, such as a NSCLC comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein to an individual having a cancer, such as a cancer provided herein (e.g., a NSCLC), e.g., comprising a deletion of a CDKN2A gene, or of a portion thereof, and/or a deletion of a CDKN2B gene, or of a portion thereof, and identified in the report. In some embodiments, the report includes information on the likely effectiveness, acceptability, and/or advisability of applying a therapeutic option (e.g., such as an anti-cancer therapy provided herein, or a treatment selected or identified according to the methods provided herein to an individual having a cancer, such as a cancer provided herein (e.g., a NSCLC), e.g., comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein (e.g., a NSCLC); detecting a deletion of an MTAP gene, or of a portion thereof, and one or more mutations in an EGFR gene or in a SMARCA4 gene in the sample, or acquiring knowledge of the presence of a deletion of an MTAP gene, or of a portion thereof, and one or more mutations in an EGFR gene or in a SMARCA4 gene in the sample; and generating a report. In some embodiments, the method comprises detecting a deletion of a CDKN2A gene, or of a portion thereof, and/or a deletion of a CDKN2B gene, or of a portion thereof, in the sample, or acquiring knowledge of the presence of a deletion of a CDKN2A gene, or of a portion thereof, and/or a deletion of a CDKN2B gene, or of a portion thereof, in the sample. In some embodiments, the method comprises detecting one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, or acquiring knowledge of the presence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, a report generated according to the methods provided herein comprises one or more of: information about the presence or absence of a deletion of an MTAP gene, or of a portion thereof, and one or more mutations in an EGFR gene or in a SMARCA4 gene in the sample; an identifier for the individual from which the sample was obtained; information on the role of a deletion of an MTAP gene, or of a portion thereof, and one or more mutations in an EGFR gene or in a SMARCA4 gene, or the corresponding wild type counterparts, in disease (e.g., such as in cancer, e.g., a NSCLC); information on prognosis, resistance, or potential or suggested therapeutic options (such as an anti-cancer therapy provided herein, or a treatment selected or identified according to the methods provided herein, such as an EGFR-targeted therapy or a SMARCA4-targeted therapy); information on the likely effectiveness, acceptability, or the advisability of applying a therapeutic option (such as an anti-cancer therapy provided herein, or a treatment selected or identified according to the methods provided herein, e.g., an EGFR-targeted therapy or a SMARCA4-targeted therapy) to the individual; a recommendation or information on the administration of a treatment (such as an anti-cancer therapy provided herein, or a treatment selected or identified according to the methods provided herein, e.g., an EGFR-targeted therapy or a SMARCA4-targeted therapy); or a recommendation or information on the dosage or treatment regimen of a treatment (such as an anti-cancer therapy provided herein, or a treatment selected or identified according to the methods provided herein, e.g., an EGFR-targeted therapy or a SMARCA4-targeted therapy), e.g., in combination with other treatments (e.g., a second therapeutic agent). In some embodiments, the report comprises information about the presence or absence of a deletion of a CDKN2A gene, or of a portion thereof, and/or a deletion of a CDKN2B gene, or of a portion thereof, in the sample. In some embodiments, the report comprises information about the presence or absence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., a NSCLC, such as a NSCLC comprising a deletion of an MTAP gene, or of a portion thereof, and one or more mutations in an EGFR gene or in a SMARCA4 gene), and optionally a deletion of a CDKN2A gene, or of a portion thereof, and/or a deletion of a CDKN2B gene, or of a portion thereof, or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a deletion of an MTAP gene, or of a portion thereof, and one or more mutations in an EGFR gene or in a SMARCA4 gene, and optionally a deletion of a CDKN2A gene, or of a portion thereof, and/or a deletion of a CDKN2B gene, or of a portion thereof, in a sample obtained from an individual (e.g., an individual having a cancer, e.g., a NSCLC). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a deletion of an MTAP gene, or of a portion thereof, and one or more mutations in an EGFR gene or in a SMARCA4 gene, and optionally a deletion of a CDKN2A gene, or of a portion thereof, and/or a deletion of a CDKN2B gene, or of a portion thereof, in a sample obtained from an individual (e.g., an individual having a cancer). The report may be provided to an individual or a patient, e.g., an individual or a patient with a cancer, such as a cancer provided herein, e.g., a NSCLC, such as a NSCLC comprising a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB in a sample obtained from an individual (e.g., an individual having a cancer, e.g., a NSCLC). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual (e.g., an individual having a cancer).

In some embodiments of any of the reports or methods provided herein, the potential or suggested therapeutic options include a PRMT5-targeted therapy and an additional anti-cancer therapy, e.g., an anti-cancer therapy provided herein, an EGFR-targeted therapy or a SMARCA4-targeted therapy. In some embodiments, the report includes information on the likely effectiveness, acceptability, and/or advisability of applying a PRMT5-targeted therapy and an additional anti-cancer therapy, e.g., an anti-cancer therapy provided herein, an EGFR-targeted therapy or a SMARCA4-targeted therapy, to an individual having a cancer, such as a cancer provided herein (e.g., a NSCLC), e.g., comprising a deletion of an MTAP gene, or of a portion thereof, and one or more mutations in an EGFR gene or in a SMARCA4 gene, and identified in the report. In some embodiments, the report includes information or a recommendation on the administration of a PRMT5-targeted therapy and an additional anti-cancer therapy, e.g., an EGFR-targeted therapy or a SMARCA4-targeted therapy. In some embodiments, the report includes information on the likely effectiveness, acceptability, and/or advisability of applying a PRMT5-targeted therapy and an additional anti-cancer therapy, e.g., an anti-cancer therapy provided herein, to an individual having a cancer, such as a cancer provided herein (e.g., a NSCLC), e.g., comprising a deletion of an MTAP gene, or of a portion thereof, and one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, and identified in the report. In some embodiments, the report includes information or a recommendation on the administration of a PRMT5-targeted therapy and an additional anti-cancer therapy, e.g., an anti-cancer therapy provided herein.

### MTAP-Intact Non-Small Cell Lung Cancer

In some aspects, provided herein are methods of treating or delaying progression of a non-small cell lung cancer (NSCLC) in an individual, wherein the NSCLC does not comprise a deletion of an methylthioadenosine phosphorylase (MTAP) gene, or of a portion thereof (MTAP-intact NSCLC).

In other aspects, methods are provided for identifying one or more treatment options for an individual having an MTAP-intact NSCLC. In other aspects, methods are provided for selecting treatment for an individual having an MTAP-intact NSCLC. In some aspects, methods are provided for predicting survival of an individual having an MTAP-intact NSCLC treated with an anti-cancer therapy. In other aspects, provided herein are methods of identifying an individual having an MTAP-intact NSCLC, who may benefit from a treatment comprising an anti-cancer therapy. In other aspects, provided herein are methods of selecting a therapy for an individual having an MTAP-intact NSCLC. In other aspects, provided herein are methods of assessing, screening, or diagnosing an individual having an MTAP-intact NSCLC.

In some embodiments, the methods comprise acquiring knowledge of or detecting in a sample from an individual having an MTAP-intact NSCLC a tumor mutational burden (TMB) of at least about 10 mut/Mb or at least about 20 mut/Mb, a PD-L1-positive NSCLC, or one or more mutations in an RB1 gene, a KRAS gene, or in a TP53 gene. In some embodiments, the methods comprise acquiring knowledge of or detecting in a sample from an individual having an MTAP-intact NSCLC, a tumor mutational burden (TMB) of at least about 10 mut/Mb or at least about 20 mut/Mb, a PD-L1-positive NSCLC, or one or more mutations in one or more genes, wherein the one or more genes comprise CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1.

Methods of Selecting or Identifying a Treatment

In some aspects, provided herein are methods of identifying an individual having cancer, e.g., a NSCLC, who may benefit from an anti-cancer therapy, e.g., an anti-cancer therapy provided herein. In some embodiments, the methods comprise detecting a TMB of at least about 10 mut/Mb or at least about 20 mut/Mb, a PD-L1-positive status, and/or one or more mutations in an RB1 gene, a KRAS gene, or a TP53 gene in a sample obtained from the individual. In some embodiments, the methods comprise acquiring knowledge of the presence of a TMB of at least about 10 mut/Mb or at least about 20 mut/Mb, a PD-L1-positive status, and/or one or more mutations in an RB1 gene, a KRAS gene, or a TP53 gene in a sample obtained from the individual. In some embodiments, the presence of the TMB of at least about 10 mut/Mb or at least about 20 mut/Mb, a PD-L1-positive status, and/or one or more mutations in an RB1 gene, a KRAS gene, or a TP53 gene in the sample identifies the individual as one who may benefit from a treatment comprising an anti-cancer therapy, e.g., an anti-cancer therapy provided herein, such as an immunotherapy, an RB1-targeted therapy, a KRAS-targeted therapy or a TP53-targeted therapy. In some embodiments, detection of the TMB of at least about 10 mut/Mb or at least about 20 mut/Mb, the PD-L1-positive status, and/or one or more mutations in an RB1 gene, a KRAS gene, or a TP53 gene in the sample identifies the individual as one who may benefit from a treatment comprising an anti-cancer therapy, e.g., an anti-cancer therapy provided herein, such as an immunotherapy, an RB1-targeted therapy, a KRAS-targeted therapy or a TP53-targeted therapy. In some embodiments, responsive to knowledge of a TMB of at least about 10 mut/Mb or at least about 20 mut/Mb, a PD-L1-positive status, and/or one or more mutations in an RB1 gene, a KRAS gene, or a TP53 gene in the sample, the individual is identified as one who may benefit from a treatment comprising an anti-cancer therapy, e.g., an anti-cancer therapy provided herein, such as an immunotherapy, an RB1-targeted therapy, a KRAS-targeted therapy or a TP53-targeted therapy. In some embodiments, the presence of a TMB of at least about 10 mut/Mb or at least about 20 mut/Mb and/or a PD-L1-positive status in the sample identifies the individual as one who may benefit from a treatment comprising an anti-cancer therapy, e.g., an anti-cancer therapy provided herein, such as an immunotherapy. In some embodiments, detection of a TMB of at least about 10 mut/Mb or at least about 20 mut/Mb and/or a PD-L1-positive status in the sample identifies the individual as one who may benefit from a treatment comprising an anti-cancer therapy, e.g., an anti-cancer therapy provided herein, such as an immunotherapy. In some embodiments, responsive to knowledge of the TMB of at least about 10 mut/Mb or at least about 20 mut/Mb and/or a PD-L1-positive status in the sample, the individual is identified as one who may benefit from a treatment comprising an anti-cancer therapy, e.g., an anti-cancer therapy provided herein, such as an immunotherapy. In some embodiments, the presence of one or more mutations in an RB1 gene in the sample identifies the individual as one who may benefit from a treatment comprising an anti-cancer therapy, e.g., an anti-cancer therapy provided herein, such as an RB1-targeted therapy. In some embodiments, detection of one or more mutations in an RB1 gene in the sample identifies the individual as one who may benefit from a treatment comprising an anti-cancer therapy, e.g., an anti-cancer therapy provided herein, such as an RB1-targeted therapy. In some embodiments, responsive to knowledge of one or more mutations in an RB1 gene in the sample, the individual is identified as one who may benefit from a treatment comprising an anti-cancer therapy, e.g., an anti-cancer therapy provided herein, such as an RB1-targeted therapy. In some embodiments, the presence of one or more mutations in a KRAS gene in the sample identifies the individual as one who may benefit from a treatment comprising an anti-cancer therapy, e.g., an anti-cancer therapy provided herein, such as a KRAS-targeted therapy. In some embodiments, detection of one or more mutations in a KRAS gene in the sample identifies the individual as one who may benefit from a treatment comprising an anti-cancer therapy, e.g., an anti-cancer therapy provided herein, such as a KRAS-targeted therapy. In some embodiments, responsive to knowledge of one or more mutations in a KRAS gene in the sample, the individual is identified as one who may benefit from a treatment comprising an anti-cancer therapy, e.g., an anti-cancer therapy provided herein, such as a KRAS-targeted therapy. In some embodiments, the presence of one or more mutations in a TP53 gene in the sample identifies the individual as one who may benefit from a treatment comprising an anti-cancer therapy, e.g., an anti-cancer therapy provided herein, such as a TP53-targeted therapy. In some embodiments, detection of one or more mutations in a TP53 gene in the sample identifies the individual as one who may benefit from a treatment comprising an anti-cancer therapy, e.g., an anti-cancer therapy provided herein, such as a TP53-targeted therapy. In some embodiments, responsive to knowledge of one or more mutations in a TP53 gene in the sample, the individual is identified as one who may benefit from a treatment comprising an anti-cancer therapy, e.g., an anti-cancer therapy provided herein, such as a TP53-targeted therapy. In some embodiments, the TMB of at least about 10 mut/Mb or at least about 20 mut/Mb, the PD-L1-positive status, and/or one or more mutations in an RB1 gene, a KRAS gene, or a TP53 gene are detected using any suitable method known in the art or described herein. In some embodiments, the sample is a sample described herein. In some embodiments, the sample comprises cells from the cancer or is obtained from cells from the cancer, e.g., a NSCLC. In some embodiments, the one or more mutations in the KRAS gene result in a G12C amino acid substitution in a KRAS polypeptide encoded by the KRAS gene.

Also provided herein are methods of identifying or selecting a treatment, a therapy, or one or more treatment options for an individual having a cancer, such as a cancer described herein, e.g., a NSCLC. In some embodiments, the cancer comprises a TMB of at least about 10 mut/Mb or at least about 20 mut/Mb, a PD-L1-positive status, and/or one or more mutations in an RB1 gene, a KRAS gene, or a TP53 gene. In some embodiments, the methods comprise detecting a TMB of at least about 10 mut/Mb or at least about 20 mut/Mb, a PD-L1-positive status, and/or one or more mutations in an RB1 gene, a KRAS gene, or a TP53 gene in a sample obtained from an individual having a cancer, such as a cancer described herein, e.g., a NSCLC. In some embodiments, the TMB of at least about 10 mut/Mb or at least about 20 mut/Mb, the PD-L1-positive status, and/or one or more mutations in an RB1 gene, a KRAS gene, or a TP53 gene are detected using any suitable method known in the art or described herein. In some embodiments, detection of a TMB of at least about 10 mut/Mb or at least about 20 mut/Mb, a PD-L1-positive status, and/or one or more mutations in an RB1 gene, a KRAS gene, or a TP53 gene in a sample obtained from an individual having a cancer, e.g., a cancer described herein, e.g., a NSCLC, identifies the individual as one who may benefit from an anti-cancer therapy, e.g., an anti-cancer therapy provided herein, such as an immunotherapy, an RB1-targeted therapy, a KRAS-targeted therapy or a TP53-targeted therapy. In some embodiments, the presence of a TMB of at least about 10 mut/Mb or at least about 20 mut/Mb, a PD-L1-positive status, and/or one or more mutations in an RB1 gene, a KRAS gene, or a TP53 gene in a sample obtained from an individual having a cancer, e.g., a cancer provided herein, e.g., a NSCLC, identifies the individual as one who may benefit from an anti-cancer therapy, such as an anti-cancer therapy provided herein, e.g., an immunotherapy, an RB1-targeted therapy, a KRAS-targeted therapy or a TP53-targeted therapy. In some embodiments, responsive to detection of a TMB of at least about 10 mut/Mb or at least about 20 mut/Mb, a PD-L1-positive status, and/or one or more mutations in an RB1 gene, a KRAS gene, or a TP53 gene in a sample obtained from an individual having a cancer, e.g., a cancer provided herein, e.g., a NSCLC, the individual is classified as a candidate to receive an anti-cancer therapy, such as an anti-cancer therapy provided herein, e.g., an immunotherapy, an RB1-targeted therapy, a KRAS-targeted therapy or a TP53-targeted therapy. In some embodiments, responsive to detection of a TMB of at least about 10 mut/Mb or at least about 20 mut/Mb, a PD-L1-positive status, and/or one or more mutations in an RB1 gene, a KRAS gene, or a TP53 gene in a sample obtained from an individual having a cancer, e.g., a cancer provided herein, e.g., a NSCLC, the individual is classified or identified as likely to respond to an anti-cancer therapy, such as an anti-cancer therapy provided herein, e.g., an immunotherapy, an RB1-targeted therapy, a KRAS-targeted therapy or a TP53-targeted therapy. In some embodiments, detection of a TMB of at least about 10 mut/Mb or at least about 20 mut/Mb and/or a PD-L1-positive status in a sample obtained from an individual having a cancer, e.g., a cancer described herein, e.g., a NSCLC, identifies the individual as one who may benefit from an anti-cancer therapy, e.g., an anti-cancer therapy provided herein, such as an immunotherapy. In some embodiments, the presence of a TMB of at least about 10 mut/Mb or at least about 20 mut/Mb and/or a PD-L1-positive status in a sample obtained from an individual having a cancer, e.g., a cancer provided herein, e.g., a NSCLC, identifies the individual as one who may benefit from an anti-cancer therapy, such as an anti-cancer therapy provided herein, e.g., an immunotherapy. In some embodiments, responsive to detection of a TMB of at least about 10 mut/Mb or at least about 20 mut/Mb and/or a PD-L1-positive status in a sample obtained from an individual having a cancer, e.g., a cancer provided herein, e.g., a NSCLC, the individual is classified as a candidate to receive an anti-cancer therapy, such as an anti-cancer therapy provided herein, e.g., an immunotherapy. In some embodiments, responsive to detection of a TMB of at least about 10 mut/Mb or at least about 20 mut/Mb and/or a PD-L1-positive status in a sample obtained from an individual having a cancer, e.g., a cancer provided herein, e.g., a NSCLC, the individual is classified or identified as likely to respond to an anti-cancer therapy, such as an anti-cancer therapy provided herein, e.g., an immunotherapy. In some embodiments, detection of one or more mutations in an RB1 gene in a sample obtained from an individual having a cancer, e.g., a cancer described herein, e.g., a NSCLC, identifies the individual as one who may benefit from an anti-cancer therapy, e.g., an anti-cancer therapy provided herein, such as an RB1-targeted therapy. In some embodiments, the presence of one or more mutations in an RB1 gene in a sample obtained from an individual having a cancer, e.g., a cancer provided herein, e.g., a NSCLC, identifies the individual as one who may benefit from an anti-cancer therapy, such as an anti-cancer therapy provided herein, e.g., an RB1-targeted therapy. In some embodiments, responsive to detection of one or more mutations in an RB1 gene in a sample obtained from an individual having a cancer, e.g., a cancer provided herein, e.g., a NSCLC, the individual is classified as a candidate to receive an anti-cancer therapy, such as an anti-cancer therapy provided herein, e.g., an RB1-targeted therapy. In some embodiments, responsive to detection of one or more mutations in an RB1 gene in a sample obtained from an individual having a cancer, e.g., a cancer provided herein, e.g., a NSCLC, the individual is classified or identified as likely to respond to an anti-cancer therapy, such as an anti-cancer therapy provided herein, e.g., an RB1-targeted therapy. In some embodiments, the sample is a sample described herein. In some embodiments, detection of one or more mutations in a KRAS gene in a sample obtained from an individual having a cancer, e.g., a cancer described herein, e.g., a NSCLC, identifies the individual as one who may benefit from an anti-cancer therapy, e.g., an anti-cancer therapy provided herein, such as a KRAS-targeted therapy. In some embodiments, the presence of one or more mutations in a KRAS gene in a sample obtained from an individual having a cancer, e.g., a cancer provided herein, e.g., a NSCLC, identifies the individual as one who may benefit from an anti-cancer therapy, such as an anti-cancer therapy provided herein, e.g., a KRAS-targeted therapy. In some embodiments, responsive to detection of one or more mutations in a KRAS gene in a sample obtained from an individual having a cancer, e.g., a cancer provided herein, e.g., a NSCLC, the individual is classified as a candidate to receive an anti-cancer therapy, such as an anti-cancer therapy provided herein, e.g., a KRAS-targeted therapy. In some embodiments, responsive to detection of one or more mutations in a KRAS gene in a sample obtained from an individual having a cancer, e.g., a cancer provided herein, e.g., a NSCLC, the individual is classified or identified as likely to respond to an anti-cancer therapy, such as an anti-cancer therapy provided herein, e.g., a KRAS-targeted therapy. In some embodiments, the sample is a sample described herein. In some embodiments, detection of one or more mutations in a TP53 gene in a sample obtained from an individual having a cancer, e.g., a cancer described herein, e.g., a NSCLC, identifies the individual as one who may benefit from an anti-cancer therapy, e.g., an anti-cancer therapy provided herein, such as a TP53-targeted therapy. In some embodiments, the presence of one or more mutations in a TP53 gene in a sample obtained from an individual having a cancer, e.g., a cancer provided herein, e.g., a NSCLC, identifies the individual as one who may benefit from an anti-cancer therapy, such as an anti-cancer therapy provided herein, e.g., a TP53-targeted therapy. In some embodiments, responsive to detection of one or more mutations in a TP53 gene in a sample obtained from an individual having a cancer, e.g., a cancer provided herein, e.g., a NSCLC, the individual is classified as a candidate to receive an anti-cancer therapy, such as an anti-cancer therapy provided herein, e.g., a TP53-targeted therapy. In some embodiments, responsive to detection of one or more mutations in a TP53 gene in a sample obtained from an individual having a cancer, e.g., a cancer provided herein, e.g., a NSCLC, the individual is classified or identified as likely to respond to an anti-cancer therapy, such as an anti-cancer therapy provided herein, e.g., a TP53-targeted therapy. In some embodiments, the sample is a sample described herein. In some embodiments, the sample comprises cells from the cancer or is obtained from cells from the cancer, e.g., a NSCLC. In some embodiments, the methods further comprise generating a report, e.g., as described herein. In some embodiments, the report comprises a treatment, a therapy, or one or more treatment options identified or selected for the individual, e.g., based at least in part on detection of a TMB of at least about 10 mut/Mb or at least about 20 mut/Mb, a PD-L1-positive status, and/or one or more mutations in an RB1 gene, a KRAS gene, or a TP53 gene in the sample. In some embodiments, the one or more mutations in a KRAS gene comprise a G12C amino acid substitution in a KRAS polypeptide encoded by the KRAS gene. In some embodiments, the one or more mutations in the KRAS gene result in a G12C amino acid substitution in a KRAS polypeptide encoded by the KRAS gene.

In some aspects, provided herein are methods of identifying an individual having cancer, e.g., a NSCLC, who may benefit from an anti-cancer therapy, e.g., an anti-cancer therapy provided herein. In some embodiments, the methods comprise detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, the methods comprise acquiring knowledge of the presence of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, the presence of the TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the individual as one who may benefit from a treatment comprising an anti-cancer therapy, e.g., an anti-cancer therapy provided herein, such as an immunotherapy and/or another anti-cancer therapy provided herein. In some embodiments, detection of the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the individual as one who may benefit from a treatment comprising an anti-cancer therapy, e.g., an anti-cancer therapy provided herein, such as an immunotherapy and/or another anti-cancer therapy provided herein. In some embodiments, responsive to knowledge of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is identified as one who may benefit from a treatment comprising an anti-cancer therapy, e.g., an anti-cancer therapy provided herein, such as an immunotherapy and/or an anti-cancer therapy provided herein. In some embodiments, the presence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the individual as one who may benefit from a treatment comprising an anti-cancer therapy, e.g., an anti-cancer therapy provided herein. In some embodiments, detection of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the individual as one who may benefit from a treatment comprising an anti-cancer therapy, e.g., an anti-cancer therapy provided herein. In some embodiments, responsive to knowledge of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is identified as one who may benefit from a treatment comprising an anti-cancer therapy, e.g., an anti-cancer therapy provided herein. In some embodiments, the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are detected using any suitable method known in the art or described herein. In some embodiments, the sample is a sample described herein. In some embodiments, the sample comprises cells from the cancer or is obtained from cells from the cancer, e.g., a NSCLC.

Also provided herein are methods of identifying or selecting a treatment, a therapy, or one or more treatment options for an individual having a cancer, such as a cancer described herein. In some embodiments, the cancer comprises a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the methods comprise detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, orRB in a sample obtained from an individual having a cancer, such as a cancer described herein, e.g., a NSCLC. In some embodiments, the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are detected using any suitable method known in the art or described herein. In some embodiments, detection of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer described herein, e.g., a NSCLC, identifies the individual as one who may benefit from an anti-cancer therapy, e.g., an anti-cancer therapy provided herein, such as an immunotherapy and/or another anti-cancer therapy provided herein. In some embodiments, the presence of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, identifies the individual as one who may benefit from an anti-cancer therapy, such as an anti-cancer therapy provided herein, e.g., an immunotherapy and/or another anti-cancer therapy provided herein. In some embodiments, responsive to detection of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, the individual is classified as a candidate to receive an anti-cancer therapy, such as an anti-cancer therapy provided herein, e.g., an immunotherapy and/or another anti-cancer therapy provided herein. In some embodiments, responsive to detection of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, the individual is classified or identified as likely to respond to an anti-cancer therapy, such as an anti-cancer therapy provided herein, e.g., an immunotherapy and/or another anti-cancer therapy provided herein. In some embodiments, detection of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer described herein, e.g., a NSCLC, identifies the individual as one who may benefit from an anti-cancer therapy, e.g., an anti-cancer therapy provided herein. In some embodiments, the presence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, identifies the individual as one who may benefit from an anti-cancer therapy, such as an anti-cancer therapy provided herein. In some embodiments, responsive to detection of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, the individual is classified as a candidate to receive an anti-cancer therapy, such as an anti-cancer therapy provided herein. In some embodiments, responsive to detection of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual having a cancer, e.g., a cancer provided herein, e.g., a NSCLC, the individual is classified or identified as likely to respond to an anti-cancer therapy, such as an anti-cancer therapy provided herein. In some embodiments, the sample is a sample described herein. In some embodiments, the sample comprises cells from the cancer or is obtained from cells from the cancer, e.g., a NSCLC. In some embodiments, the methods further comprise generating a report, e.g., as described herein. In some embodiments, the report comprises a treatment, a therapy, or one or more treatment options identified or selected for the individual, e.g., based at least in part on detection of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample.

In some embodiments of any of the methods provided herein, the methods further comprise acquiring knowledge of or detecting in a sample from an individual the absence of a deletion of an MTAP gene, or of a portion thereof. In some embodiments of any of the methods provided herein, the individual does not have a deletion of an MTAP gene, or of a portion thereof.

Methods of Treatment

Also provided herein are methods of treating or delaying progression of a cancer in an individual, such as a cancer provided herein, e.g., a NSCLC. In some embodiments, the individual has a cancer comprising a TMB of at least about 10 mut/Mb or at least about 20 mut/Mb, a PD-L1-positive status, and/or one or more mutations in an RB1 gene, a KRAS gene, or a TP53 gene. In some embodiments, the methods of treating or delaying progression of a cancer of the disclosure, e.g., a NSCLC, comprising a TMB of at least about 10 mut/Mb or at least about 20 mut/Mb, a PD-L1-positive status, and/or one or more mutations in an RB1 gene, a KRAS gene, or a TP53 gene comprise administering to an individual a therapeutically effective amount of an anti-cancer therapy, such as an anti-cancer therapy provided herein, e.g., an immunotherapy, an RB1-targeted therapy, a KRAS-targeted therapy or a TP53-targeted therapy. In some embodiments, the methods of treating or delaying progression of a cancer of the disclosure, e.g., a NSCLC, comprising a TMB of at least about 10 mut/Mb or at least about 20 mut/Mb, a PD-L1-positive status, and/or one or more mutations in an RB1 gene, a KRAS gene, or a TP53 gene, comprise administering to an individual an effective amount of an anti-cancer therapy, such as an anti-cancer therapy provided herein (e.g., an immunotherapy, an RB1-targeted therapy, a KRAS-targeted therapy or a TP53-targeted therapy), responsive to knowledge of the presence of the TMB of at least about 10 mut/Mb or at least about 20 mut/Mb, the PD-L1-positive status, and/or one or more mutations in an RB1 gene, a KRAS gene, or a TP53 gene in a sample obtained from the individual. In some embodiments, the one or more mutations in the KRAS gene result in a G12C amino acid substitution in a KRAS polypeptide encoded by the KRAS gene. In some embodiments, the sample is a sample described herein. In some embodiments, the sample comprises cells from the cancer or is obtained from cells from the cancer, e.g. a NSCLC. The methods of treatment disclosed herein may include any of the anti-cancer therapies and/or therapeutic agents described herein, e.g., infra.

In some embodiments, the individual has a cancer comprising a TMB of at least about 10 mut/Mb or at least about 20 mut/Mb and/or a PD-L1-positive status. In some embodiments, the methods of treating or delaying progression of a cancer of the disclosure, e.g., a NSCLC, comprising a TMB of at least about 10 mut/Mb or at least about 20 mut/Mb and/or a PD-L1-positive status, comprise administering to an individual a therapeutically effective amount of an anti-cancer therapy, such as an anti-cancer therapy provided herein, e.g., an immunotherapy. In some embodiments, the methods of treating or delaying progression of a cancer of the disclosure, e.g., a NSCLC comprising a TMB of at least about 10 mut/Mb or at least about 20 mut/Mb and/or a PD-L1-positive status, comprise administering to an individual an effective amount of an anti-cancer therapy, such as an anti-cancer therapy provided herein (e.g., an immunotherapy), responsive to knowledge of the presence of the TMB of at least about 10 mut/Mb or at least about 20 mut/Mb and/or the PD-L1-positive status in a sample obtained from the individual. In some embodiments, the immunotherapy comprises one or more of a checkpoint inhibitor (e.g., a checkpoint inhibitor provided herein), a cancer vaccine (e.g., a cancer vaccine provided herein), a cell-based therapy (e.g., a cell-based therapy provided herein), a T cell receptor (TCR)-based therapy (e.g., a TCR-based therapy provided herein), an adjuvant immunotherapy (e.g., an adjuvant immunotherapy provided herein), a cytokine immunotherapy (e.g., a cytokine immunotherapy provided herein), or an oncolytic virus therapy (e.g., an oncolytic virus therapy provided herein). In some embodiments, the anti-cancer therapy is administered in combination with a second anti-cancer therapy or agent, such as any of the anti-cancer agents or therapies described herein.

In some embodiments, the individual has a cancer comprising one or more mutations in an RB1 gene. In some embodiments, the methods of treating or delaying progression of a cancer of the disclosure, e.g., a NSCLC comprising one or more mutations in an RB1 gene, comprise administering to an individual a therapeutically effective amount of an anti-cancer therapy, such as an anti-cancer therapy provided herein, e.g., an RB1-targeted therapy. In some embodiments, the methods of treating or delaying progression of a cancer of the disclosure, e.g., a NSCLC comprising one or more mutations in an RB1 gene, comprise administering to the individual an effective amount of an anti-cancer therapy, such as an anti-cancer therapy provided herein (e.g., an RB1-targeted therapy), responsive to knowledge of the presence of the one or more mutations in an RB1 gene in a sample obtained from the individual. In some embodiments, the RB1-targeted therapy comprises one or more of a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor. In some embodiments, the anti-cancer therapy is administered in combination with a second anti-cancer therapy or agent, such as any of the anti-cancer agents or therapies described herein.

In some embodiments, the individual has a cancer comprising one or more mutations in a KRAS gene. In some embodiments, the methods of treating or delaying progression of a cancer of the disclosure, e.g., a NSCLC comprising one or more mutations in a KRAS gene, comprise administering to an individual a therapeutically effective amount of an anti-cancer therapy, such as an anti-cancer therapy provided herein, e.g., a KRAS-targeted therapy. In some embodiments, the methods of treating or delaying progression of a cancer of the disclosure, e.g., a NSCLC comprising one or more mutations in a KRAS gene, comprise administering to the individual an effective amount of an anti-cancer therapy, such as an anti-cancer therapy provided herein (e.g., a KRAS-targeted therapy), responsive to knowledge of the presence of the one or more mutations in a KRAS gene in a sample obtained from the individual. In some embodiments, the KRAS-targeted therapy comprises a kinase inhibitor. In some embodiments, the kinase inhibitor is MCP1, SCH54292, tipifarnib (R115777), lonafarnib (SCH663366), or BMS-214662. In some embodiments, the KRAS-targeted therapy comprises one or more of a farnesyltransferase inhibitor, a geranylgeranyltransferase inhibitor, a palmitoylation inhibitor, an inhibitor of methylation cleavage, or an agent that inhibits the modification or post-translational processing of KRAS. In other embodiments, the KRAS-targeted therapy comprises one or more of a Raf inhibitor, a MEK inhibitor, or an mTOR inhibitor. In some embodiments, the one or more mutations in a KRAS gene comprise a G12C amino acid substitution in a KRAS polypeptide encoded by the KRAS gene. In some embodiments, the one or more mutations in the KRAS gene result in a G12C amino acid substitution in a KRAS polypeptide encoded by the KRAS gene. In some embodiments, the KRAS-targeted therapy comprises a KRAS (G12C)-targeted therapy. In some embodiments, the KRAS (G12C)-targeted therapy comprises one or more of KRAS inhibitor and/or a SHP2 (also known as PTPN11) inhibitor.

In some embodiments, the individual has a cancer comprising one or more mutations in a TP53 gene. In some embodiments, the methods of treating or delaying progression of a cancer of the disclosure, e.g., a NSCLC comprising one or more mutations in a TP53 gene, comprise administering to an individual a therapeutically effective amount of an anti-cancer therapy, such as an anti-cancer therapy provided herein, e.g., a TP53-targeted therapy. In some embodiments, the methods of treating or delaying progression of a cancer of the disclosure, e.g., a NSCLC comprising one or more mutations in a TP53 gene, comprise administering to the individual an effective amount of an anti-cancer therapy, such as an anti-cancer therapy provided herein (e.g., a TP53-targeted therapy), responsive to knowledge of the presence of the one or more mutations in a TP53 gene in a sample obtained from the individual. In some embodiments, the TP53-targeted therapy comprises one or more of a p53 reactivator, an mTOR inhibitor, a Pin1 inhibitor, an ATR inhibitor, a proteasome inhibitor, a CHK inhibitor, an ATM inhibitor, a WEE1 inhibitor, or a murine double minute 2 (MDM2) inhibitor.

In some embodiments, the individual has a cancer, e.g., a NSCLC comprising a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the methods of treating or delaying progression of a cancer of the disclosure, e.g., a NSCLC comprising a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprise administering to an individual a therapeutically effective amount of an anti-cancer therapy, such as an anti-cancer therapy provided herein, e.g., an immunotherapy and/or another anti-cancer therapy provided herein. In some embodiments, the methods of treating or delaying progression of a cancer of the disclosure, e.g., a NSCLC comprising a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprise administering to an individual an effective amount of an anti-cancer therapy, such as an anti-cancer therapy provided herein (e.g., an immunotherapy or another anti-cancer therapy provided herein), responsive to knowledge of the presence of the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, the sample is a sample described herein. In some embodiments, the sample comprises cells from the cancer or is obtained from cells from the cancer, e.g. a NSCLC. The methods of treatment disclosed herein may include any of the anti-cancer therapies and/or therapeutic agents described herein, e.g., infra.

In some embodiments, the individual has a cancer comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the methods of treating or delaying progression of a cancer of the disclosure, e.g., a NSCLC comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprise administering to an individual a therapeutically effective amount of an anti-cancer therapy, such as an anti-cancer therapy provided herein. In some embodiments, the methods of treating or delaying progression of a cancer of the disclosure, e.g., a NSCLC comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprise administering to the individual an effective amount of an anti-cancer therapy, such as an anti-cancer therapy provided herein, responsive to knowledge of the presence of the one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, the anti-cancer therapy is administered in combination with a second anti-cancer therapy or agent, such as any of the anti-cancer agents or therapies described herein.

In some embodiments of any of the methods provided herein, the methods further comprise acquiring knowledge of or detecting in a sample from an individual the absence of a deletion of an MTAP gene, or of a portion thereof. In some embodiments of any of the methods provided herein, the individual does not have a deletion of an MTAP gene, or of a portion thereof.

Methods of Diagnosing, Assessing, Screening, Monitoring or Predicting

In some aspects, provided herein are methods of diagnosing or assessing a TMB of at least about 10 mut/Mb or at least about 20 mut/Mb, a PD-L1-positive status, and/or one or more mutations in an RB1 gene, a KRAS gene, or a TP53 gene in a sample from an individual having a cancer, such as a NSCLC. In some embodiments, the methods comprise acquiring knowledge of the presence of a TMB of at least about 10 mut/Mb or at least about 20 mut/Mb, a PD-L1-positive status, and/or one or more mutations in an RB1 gene, a KRAS gene, or a TP53 gene in a sample obtained from the individual. In some embodiments, the methods comprise detecting a TMB of at least about 10 mut/Mb or at least about 20 mut/Mb, a PD-L1-positive status, and/or one or more mutations in an RB1 gene, a KRAS gene, or a TP53 gene in a sample obtained from the individual. In some embodiments, a TMB of at least about 10 mut/Mb or at least about 20 mut/Mb, a PD-L1-positive status, and/or one or more mutations in an RB1 gene, a KRAS gene, or a TP53 gene are detected in a sample obtained from the individual using any method known in the art, such as one or more of the methods of detection of nucleic acid molecules or polypeptides described herein. In some embodiments, the methods further comprise providing a diagnosis or an assessment of the TMB of at least about 10 mut/Mb or at least about 20 mut/Mb, the PD-L1-positive status, and/or one or more mutations in an RB1 gene, a KRAS gene, or a TP53 gene. In some embodiments, the diagnosis or assessment identifies the presence or absence of a TMB of at least about 10 mut/Mb or at least about 20 mut/Mb, a PD-L1-positive status, and/or one or more mutations in an RB1 gene, a KRAS gene, or a TP53 gene in the sample. In some embodiments, the diagnosis or assessment identifies the cancer, such as a NSCLC provided herein, as likely to respond to an anti-cancer therapy, e.g., an anti-cancer therapy provided herein, such as an immunotherapy, an RB1-targeted therapy, a KRAS-targeted therapy and/or a TP53-targeted therapy. In some embodiments, the presence of a TMB of at least about 10 mut/Mb or at least about 20 mut/Mb, a PD-L1-positive status, and/or one or more mutations in an RB1 gene, a KRAS gene, or a TP53 gene in the sample identifies the cancer as likely to respond to an anti-cancer therapy, e.g., an anti-cancer therapy provided herein, such as an immunotherapy, an RB1-targeted therapy, a KRAS-targeted therapy or a TP53-targeted therapy. In some embodiments, the diagnosis or assessment identifies the presence or absence of a TMB of at least about 10 mut/Mb or at least about 20 mut/Mb and/or a PD-L1-positive status in the sample. In some embodiments, the diagnosis or assessment identifies the cancer, such as a NSCLC provided herein, as likely to respond to an anti-cancer therapy, e.g., an anti-cancer therapy provided herein, such as an immunotherapy. In some embodiments, the presence of a TMB of at least about 10 mut/Mb or at least about 20 mut/Mb and/or a PD-L1-positive status in the sample identifies the cancer as likely to respond to an anti-cancer therapy, e.g., an anti-cancer therapy provided herein, such as an immunotherapy. In some embodiments, the diagnosis or assessment identifies the presence or absence of one or more mutations in an RB1 gene in the sample. In some embodiments, the diagnosis or assessment identifies the cancer, such as a NSCLC provided herein, as likely to respond to an anti-cancer therapy, e.g., an anti-cancer therapy provided herein, such as an RB1-targeted therapy. In some embodiments, the presence of one or more mutations in an RB1 gene in the sample identifies the cancer as likely to respond to an anti-cancer therapy, e.g., an anti-cancer therapy provided herein, such as an RB1-targeted therapy. In some embodiments, the diagnosis or assessment identifies the presence or absence of one or more mutations in a KRAS gene in the sample. In some embodiments, the diagnosis or assessment identifies the cancer, such as a NSCLC provided herein, as likely to respond to an anti-cancer therapy, e.g., an anti-cancer therapy provided herein, such as a KRAS-targeted therapy. In some embodiments, the presence of one or more mutations in a KRAS gene in the sample identifies the cancer as likely to respond to an anti-cancer therapy, e.g., an anti-cancer therapy provided herein, such as a KRAS-targeted therapy. In some embodiments, the diagnosis or assessment identifies the presence or absence of one or more mutations in a TP53 gene in the sample. In some embodiments, the diagnosis or assessment identifies the cancer, such as a NSCLC provided herein, as likely to respond to an anti-cancer therapy, e.g., an anti-cancer therapy provided herein, such as a TP53-targeted therapy. In some embodiments, the presence of one or more mutations in a TP53 gene in the sample identifies the cancer as likely to respond to an anti-cancer therapy, e.g., an anti-cancer therapy provided herein, such as a TP53-targeted therapy. In some embodiments, the sample is a sample described herein. In some embodiments, the sample comprises cells from the cancer or is obtained from cells from the cancer, e.g., from a NSCLC. In some embodiments, the individual has a cancer, is suspected of having a cancer, is being tested for a cancer, is being treated for a cancer, or is being tested for a susceptibility to a cancer, e.g., a NSCLC. In some embodiments, the one or more mutations in the KRAS gene result in a G12C amino acid substitution in a KRAS polypeptide encoded by the KRAS gene.

In some aspects, provided herein are methods of diagnosing or assessing a cancer in an individual, e.g., a NSCLC. In some embodiments, the methods of diagnosing or assessing cancer comprise detecting a TMB of at least about 10 mut/Mb or at least about 20 mut/Mb, a PD-L1-positive status, and/or one or more mutations in an RB1 gene, a KRAS gene, or a TP53 gene in a sample obtained from the individual, e.g., a sample comprising cells from the cancer, e.g., a NSCLC. In some embodiments, the methods comprise detecting a TMB of at least about 10 mut/Mb or at least about 20 mut/Mb, a PD-L1-positive status, and/or one or more mutations in an RB1 gene, a KRAS gene, or a TP53 gene in a sample obtained from the individual using any method known in the art, such as one or more of the methods of detection of nucleic acid molecules or polypeptides described herein. In some embodiments, detection of a TMB of at least about 10 mut/Mb or at least about 20 mut/Mb, a PD-L1-positive status, and/or one or more mutations in an RB1 gene, a KRAS gene, or a TP53 gene in a sample obtained from the individual identifies the cancer, e.g., the NSCLC, as likely to respond to an anti-cancer therapy, e.g., an anti-cancer therapy provided herein, such as an immunotherapy, an RB1-targeted therapy, a KRAS-targeted therapy or a TP53-targeted therapy. In some embodiments, the presence of a TMB of at least about 10 mut/Mb or at least about 20 mut/Mb, a PD-L1-positive status, and/or one or more mutations in an RB1 gene, a KRAS gene, or a TP53 gene in a sample obtained from the individual identifies the cancer, e.g., the NSCLC, as likely to respond to an anti-cancer therapy, e.g., an anti-cancer therapy provided herein, such as an immunotherapy, an RB1-targeted therapy, a KRAS-targeted therapy or a TP53-targeted therapy. In some embodiments, the methods further comprise providing a diagnosis or an assessment of the cancer, e.g., the NSCLC, or of the TMB of at least about 10 mut/Mb or at least about 20 mut/Mb, the PD-L1-positive status, and/or one or more mutations in an RB1 gene, a KRAS gene, or a TP53 gene. In some embodiments, the diagnosis or assessment identifies the cancer as likely to respond to an anti-cancer therapy, e.g., an anti-cancer therapy provided herein, such as an immunotherapy, an RB1-targeted therapy, a KRAS-targeted therapy or a TP53-targeted therapy. In some embodiments, detection of a TMB of at least about 10 mut/Mb or at least about 20 mut/Mb and/or a PD-L1-positive status in a sample obtained from the individual identifies the cancer, e.g., the NSCLC, as likely to respond to an anti-cancer therapy, e.g., an anti-cancer therapy provided herein, such as an immunotherapy. In some embodiments, the presence of a TMB of at least about 10 mut/Mb or at least about 20 mut/Mb and/or a PD-L1-positive status in a sample obtained from the individual identifies the cancer, e.g., the NSCLC, as likely to respond to an anti-cancer therapy, e.g., an anti-cancer therapy provided herein, such as an immunotherapy. In some embodiments, the methods further comprise providing a diagnosis or an assessment of the cancer, e.g., the NSCLC, or of the TMB of at least about 10 mut/Mb or at least about 20 mut/Mb and/or the PD-L1-positive status. In some embodiments, the diagnosis or assessment identifies the cancer as likely to respond to an anti-cancer therapy, e.g., an anti-cancer therapy provided herein, such as an immunotherapy. In some embodiments, detection of one or more mutations in an RB1 gene in a sample obtained from the individual identifies the cancer, e.g., the NSCLC, as likely to respond to an anti-cancer therapy, e.g., an anti-cancer therapy provided herein, such as an RB1-targeted therapy. In some embodiments, the presence of one or more mutations in an RB1 gene in a sample obtained from the individual identifies the cancer, e.g., the NSCLC, as likely to respond to an anti-cancer therapy, e.g., an anti-cancer therapy provided herein, such as an RB1-targeted therapy. In some embodiments, the methods further comprise providing a diagnosis or an assessment of the cancer, e.g., the NSCLC, or of the one or more mutations in an RB1 gene. In some embodiments, the diagnosis or assessment identifies the cancer as likely to respond to an anti-cancer therapy, e.g., an anti-cancer therapy provided herein, such as an RB1-targeted therapy. In some embodiments, detection of one or more mutations in a KRAS gene in a sample obtained from the individual identifies the cancer, e.g., the NSCLC, as likely to respond to an anti-cancer therapy, e.g., an anti-cancer therapy provided herein, such as a KRAS-targeted therapy. In some embodiments, the presence of one or more mutations in a KRAS gene in a sample obtained from the individual identifies the cancer, e.g., the NSCLC, as likely to respond to an anti-cancer therapy, e.g., an anti-cancer therapy provided herein, such as a KRAS-targeted therapy. In some embodiments, the methods further comprise providing a diagnosis or an assessment of the cancer, e.g., the NSCLC, or of the one or more mutations in a KRAS gene. In some embodiments, the diagnosis or assessment identifies the cancer as likely to respond to an anti-cancer therapy, e.g., an anti-cancer therapy provided herein, such as a KRAS-targeted therapy. In some embodiments, detection of one or more mutations in a TP53 gene in a sample obtained from the individual identifies the cancer, e.g., the NSCLC, as likely to respond to an anti-cancer therapy, e.g., an anti-cancer therapy provided herein, such as a TP53-targeted therapy. In some embodiments, the presence of one or more mutations in a TP53 gene in a sample obtained from the individual identifies the cancer, e.g., the NSCLC, as likely to respond to an anti-cancer therapy, e.g., an anti-cancer therapy provided herein, such as a TP53-targeted therapy. In some embodiments, the methods further comprise providing a diagnosis or an assessment of the cancer, e.g., the NSCLC, or of the one or more mutations in a TP53 gene. In some embodiments, the diagnosis or assessment identifies the cancer as likely to respond to an anti-cancer therapy, e.g., an anti-cancer therapy provided herein, such as a TP53-targeted therapy. In some embodiments, the diagnosis or assessment identifies the presence or absence of the TMB of at least about 10 mut/Mb or at least about 20 mut/Mb, the PD-L1-positive status, and/or one or more mutations in an RB1 gene, a KRAS gene, or a TP53 gene in the sample. In some embodiments, the one or more mutations in the KRAS gene result in a G12C amino acid substitution in a KRAS polypeptide encoded by the KRAS gene.

In some aspects, provided herein are methods of predicting survival of an individual having a cancer, e.g., a cancer provided herein, e.g., a NSCLC. In some embodiments, the individual is being treated with an anti-cancer therapy, such as an anti-cancer therapy described herein. In some embodiments, the methods comprise acquiring knowledge of a TMB of at least about 10 mut/Mb or at least about 20 mut/Mb, a PD-L1-positive status, and/or one or more mutations in an RB1 gene, a KRAS gene, or a TP53 gene in a sample from the individual. In some embodiments, the methods comprise detecting a TMB of at least about 10 mut/Mb or at least about 20 mut/Mb, a PD-L1-positive status, and/or one or more mutations in an RB1 gene, a KRAS gene, or a TP53 gene in a sample from the individual. In some embodiments, responsive to acquiring knowledge of a TMB of at least about 10 mut/Mb or at least about 20 mut/Mb, a PD-L1-positive status, and/or one or more mutations in an RB1 gene, a KRAS gene, or a TP53 gene in the sample, the individual is predicted to have longer survival after treatment with an anti-cancer therapy, e.g., an anti-cancer therapy provided herein (e.g., an immunotherapy, an RB1-targeted therapy, a KRAS-targeted therapy or a TP53-targeted therapy), for example, as compared to an individual whose cancer does not exhibit the TMB of at least about 10 mut/Mb or at least about 20 mut/Mb, the PD-L1-positive status, and/or one or more mutations in an RB1 gene, a KRAS gene, or a TP53 gene. In some embodiments, responsive to detecting a TMB of at least about 10 mut/Mb or at least about 20 mut/Mb, a PD-L1-positive status, and/or one or more mutations in an RB1 gene, a KRAS gene, or a TP53 gene in the sample, the individual is predicted to have longer survival after treatment with an anti-cancer therapy, e.g., an anti-cancer therapy provided herein (e.g., an immunotherapy, an RB1-targeted therapy, a KRAS-targeted therapy or a TP53-targeted therapy), for example, as compared to an individual whose cancer does not exhibit the TMB of at least about 10 mut/Mb or at least about 20 mut/Mb, the PD-L1-positive status, and/or one or more mutations in an RB1 gene, a KRAS gene, or a TP53 gene. In some embodiments, responsive to acquiring knowledge of a TMB of at least about 10 mut/Mb or at least about 20 mut/Mb and/or a PD-L1-positive status in the sample, the individual is predicted to have longer survival after treatment with an anti-cancer therapy, e.g., an anti-cancer therapy provided herein (e.g., an immunotherapy), for example, as compared to an individual whose cancer does not exhibit the TMB of at least about 10 mut/Mb or at least about 20 mut/Mb and/or the PD-L1-positive status. In some embodiments, responsive to detecting a TMB of at least about 10 mut/Mb or at least about 20 mut/Mb and/or a PD-L1-positive status in the sample, the individual is predicted to have longer survival after treatment with an anti-cancer therapy, e.g., an anti-cancer therapy provided herein (e.g., an immunotherapy), for example, as compared to an individual whose cancer does not exhibit the TMB of at least about 10 mut/Mb or at least about 20 mut/Mb and/or the PD-L1-positive status. In some embodiments, responsive to acquiring knowledge of one or more mutations in an RB1 gene in the sample, the individual is predicted to have longer survival after treatment with an anti-cancer therapy, e.g., an anti-cancer therapy provided herein (e.g., an RB1-targeted therapy), for example, as compared to an individual whose cancer does not exhibit the one or more mutations in an RB1 gene. In some embodiments, responsive to detecting one or more mutations in an RB1 gene in the sample, the individual is predicted to have longer survival after treatment with an anti-cancer therapy, e.g., an anti-cancer therapy provided herein (e.g., an RB1-targeted therapy), for example, as compared to an individual whose cancer does not exhibit the one or more mutations in an RB1 gene. In some embodiments, responsive to acquiring knowledge of one or more mutations in a KRAS gene in the sample, the individual is predicted to have longer survival after treatment with an anti-cancer therapy, e.g., an anti-cancer therapy provided herein (e.g., a KRAS-targeted therapy), for example, as compared to an individual whose cancer does not exhibit the one or more mutations in a KRAS gene. In some embodiments, responsive to detecting one or more mutations in a KRAS gene in the sample, the individual is predicted to have longer survival after treatment with an anti-cancer therapy, e.g., an anti-cancer therapy provided herein (e.g., a KRAS-targeted therapy), for example, as compared to an individual whose cancer does not exhibit the one or more mutations in a KRAS gene. In some embodiments, responsive to acquiring knowledge of one or more mutations in a TP53 gene in the sample, the individual is predicted to have longer survival after treatment with an anti-cancer therapy, e.g., an anti-cancer therapy provided herein (e.g., a TP53-targeted therapy), for example, as compared to an individual whose cancer does not exhibit the one or more mutations in a TP53 gene. In some embodiments, responsive to detecting one or more mutations in a TP53 gene in the sample, the individual is predicted to have longer survival after treatment with an anti-cancer therapy, e.g., an anti-cancer therapy provided herein (e.g., a TP53-targeted therapy), for example, as compared to an individual whose cancer does not exhibit the one or more mutations in a TP53 gene. In some embodiments, the methods further comprise providing a diagnosis or an assessment. In some embodiments, the diagnosis or assessment identifies the presence or absence of the TMB of at least about 10 mut/Mb or at least about 20 mut/Mb, the PD-L1-positive status, and/or one or more mutations in an RB1 gene, a KRAS gene, or a TP53 gene in the sample. In some embodiments, the diagnosis or assessment identifies the individual as being predicted to have longer survival after treatment with an anti-cancer therapy, e.g., an anti-cancer therapy provided herein (e.g., an immunotherapy, an RB1-targeted therapy, a KRAS-targeted therapy or a TP53-targeted therapy), for example, as compared to an individual whose cancer does not exhibit the TMB of at least about 10 mut/Mb or at least about 20 mut/Mb, the PD-L1-positive status, and/or one or more mutations in an RB1 gene, a KRAS gene, or a TP53 gene. In some embodiments, the diagnosis or assessment identifies the individual as being predicted to have longer survival after treatment with an anti-cancer therapy, e.g., an anti-cancer therapy provided herein (e.g., an immunotherapy), for example, as compared to an individual whose cancer does not exhibit the TMB of at least about 10 mut/Mb or at least about 20 mut/Mb and/or the PD-L1-positive status. In some embodiments, the diagnosis or assessment identifies the individual as being predicted to have longer survival after treatment with an anti-cancer therapy, e.g., an anti-cancer therapy provided herein (e.g., an RB1-targeted therapy), for example, as compared to an individual whose cancer does not exhibit the one or more mutations in an RB1 gene. In some embodiments, the diagnosis or assessment identifies the individual as being predicted to have longer survival after treatment with an anti-cancer therapy, e.g., an anti-cancer therapy provided herein (e.g., a KRAS-targeted therapy), for example, as compared to an individual whose cancer does not exhibit the one or more mutations in a KRAS gene. In some embodiments, the diagnosis or assessment identifies the individual as being predicted to have longer survival after treatment with an anti-cancer therapy, e.g., an anti-cancer therapy provided herein (e.g., a TP53-targeted therapy), for example, as compared to an individual whose cancer does not exhibit the one or more mutations in a TP53 gene. In some embodiments, the sample is a sample described herein. In some embodiments, the sample comprises cells from the cancer or is obtained from cells from the cancer, e.g., the NSCLC. In some embodiments, the one or more mutations in the KRAS gene result in a G12C amino acid substitution in a KRAS polypeptide encoded by the KRAS gene.

In some aspects, provided herein are methods of screening an individual having cancer, suspected of having cancer, being tested for cancer, being treated for cancer, or being tested for a susceptibility to cancer, e.g., a NSCLC. In some embodiments, the individual is being treated with an anti-cancer therapy, such as an anti-cancer therapy described herein. In some embodiments, the methods comprise acquiring knowledge of a TMB of at least about 10 mut/Mb or at least about 20 mut/Mb, a PD-L1-positive status, and/or one or more mutations in an RB1 gene, a KRAS gene, or a TP53 gene in a sample from the individual. In some embodiments, the methods comprise detecting a TMB of at least about 10 mut/Mb or at least about 20 mut/Mb, a PD-L1-positive status, and/or one or more mutations in an RB1 gene, a KRAS gene, or a TP53 gene in a sample from the individual. In some embodiments, responsive to acquiring knowledge of a TMB of at least about 10 mut/Mb or at least about 20 mut/Mb, a PD-L1-positive status, and/or one or more mutations in an RB1 gene, a KRAS gene, or a TP53 gene in the sample, the individual is predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, the PD-L1-positive status, and/or one or more mutations in an RB1 gene, a KRAS gene, or a TP53 gene. In some embodiments, responsive to detecting a TMB of at least about 10 mut/Mb or at least about 20 mut/Mb, a PD-L1-positive status, and/or one or more mutations in an RB1 gene, a KRAS gene, or a TP53 gene in the sample, the individual is predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, a PD-L1-positive status, and/or one or more mutations in an RB1 gene, a KRAS gene, or a TP53 gene. In some embodiments, the methods further comprise providing a diagnosis or an assessment. In some embodiments, the diagnosis or assessment identifies the presence or absence of the TMB of at least about 10 mut/Mb or at least about 20 mut/Mb, the PD-L1-positive status, and/or one or more mutations in an RB1 gene, a KRAS gene, or a TP53 gene in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, the PD-L1-positive status, and/or one or more mutations in an RB1 gene, a KRAS gene, or a TP53 gene. In some embodiments, the sample is a sample described herein. In some embodiments, the sample comprises cells from the cancer or is obtained from cells from the cancer, e.g., the NSCLC.

In some embodiments, the methods further comprise selectively enriching for one or more nucleic acids comprising nucleotide sequences that comprise one or more mutations in an RB1 gene, a KRAS gene, or a TP53 gene to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise one or more mutations in an RB1 gene, a KRAS gene, or a TP53 gene to produce an enriched sample, e.g., using a reagent known in the art or provided herein, such as an antibody described herein. In some embodiments, the one or more mutations in the KRAS gene result in a G12C amino acid substitution in a KRAS polypeptide encoded by the KRAS gene.

In some aspects, provided herein are methods of diagnosing or assessing a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from an individual having a cancer, such as a NSCLC. In some embodiments, the methods comprise acquiring knowledge of the presence of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, the methods comprise detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual. In some embodiments, a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are detected in a sample obtained from the individual using any method known in the art, such as one or more of the methods of detection of nucleic acid molecules or polypeptides described herein. In some embodiments, the methods further comprise providing a diagnosis or an assessment of the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the diagnosis or assessment identifies the presence or absence of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, the diagnosis or assessment identifies the cancer, such as a NSCLC provided herein, as likely to respond to an anti-cancer therapy, e.g., an anti-cancer therapy provided herein, such as an immunotherapy and/or another anti-cancer therapy provided herein. In some embodiments, the presence of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the cancer as likely to respond to an anti-cancer therapy, e.g., an anti-cancer therapy provided herein, such as an immunotherapy and/or another anti-cancer therapy provided herein. In some embodiments, the diagnosis or assessment identifies the presence or absence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, the diagnosis or assessment identifies the cancer, such as a NSCLC provided herein, as likely to respond to an anti-cancer therapy, e.g., an anti-cancer therapy provided herein. In some embodiments, the presence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample identifies the cancer as likely to respond to an anti-cancer therapy, e.g., an anti-cancer therapy provided herein. In some embodiments, the sample is a sample described herein. In some embodiments, the sample comprises cells from the cancer or is obtained from cells from the cancer, e.g., from a NSCLC. In some embodiments, the individual has a cancer, is suspected of having a cancer, is being tested for a cancer, is being treated for a cancer, or is being tested for a susceptibility to a cancer, e.g., a NSCLC.

In some aspects, provided herein are methods of diagnosing or assessing a cancer in an individual, e.g., a NSCLC. In some embodiments, the methods of diagnosing or assessing cancer comprise detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual, e.g., a sample comprising cells from the cancer, e.g., a NSCLC. In some embodiments, the methods comprise detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual using any method known in the art, such as one or more of the methods of detection of nucleic acid molecules or polypeptides described herein. In some embodiments, detection of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual identifies the cancer, e.g., the NSCLC, as likely to respond to an anti-cancer therapy, e.g., an anti-cancer therapy provided herein, such as an immunotherapy and/or another anti-cancer therapy provided herein. In some embodiments, the presence of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual identifies the cancer, e.g., the NSCLC, as likely to respond to an anti-cancer therapy, e.g., an anti-cancer therapy provided herein, such as an immunotherapy and/or another anti-cancer therapy provided herein. In some embodiments, the methods further comprise providing a diagnosis or an assessment of the cancer, e.g., the NSCLC, or of the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the diagnosis or assessment identifies the cancer as likely to respond to an anti-cancer therapy, e.g., an anti-cancer therapy provided herein, such as an immunotherapy and/or another anti-cancer therapy provided herein. In some embodiments, detection of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, orRB in a sample obtained from the individual identifies the cancer, e.g., the NSCLC, as likely to respond to an anti-cancer therapy, e.g., an anti-cancer therapy provided herein. In some embodiments, the presence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from the individual identifies the cancer, e.g., the NSCLC, as likely to respond to an anti-cancer therapy, e.g., an anti-cancer therapy provided herein. In some embodiments, the methods further comprise providing a diagnosis or an assessment of the cancer, e.g., the NSCLC, or of the one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the diagnosis or assessment identifies the cancer as likely to respond to an anti-cancer therapy, e.g., an anti-cancer therapy provided herein. In some embodiments, the diagnosis or assessment identifies the presence or absence of the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample.

In some aspects, provided herein are methods of predicting survival of an individual having a cancer, e.g., a cancer provided herein, e.g., a NSCLC. In some embodiments, the individual is being treated with an anti-cancer therapy, such as an anti-cancer therapy described herein. In some embodiments, the methods comprise acquiring knowledge of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from the individual. In some embodiments, the methods comprise detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from the individual. In some embodiments, responsive to acquiring knowledge of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have longer survival after treatment with an anti-cancer therapy, e.g., an anti-cancer therapy provided herein (e.g., an immunotherapy and/or another anti-cancer therapy provided herein), for example, as compared to an individual whose cancer does not exhibit the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, responsive to detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have longer survival after treatment with an anti-cancer therapy, e.g., an anti-cancer therapy provided herein (e.g., an immunotherapy and/or another anti-cancer therapy provided herein), for example, as compared to an individual whose cancer does not exhibit the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, responsive to acquiring knowledge of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have longer survival after treatment with an anti-cancer therapy, e.g., an anti-cancer therapy provided herein, for example, as compared to an individual whose cancer does not exhibit the one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, responsive to detecting one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have longer survival after treatment with an anti-cancer therapy, e.g., an anti-cancer therapy provided herein, for example, as compared to an individual whose cancer does not exhibit the one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the methods further comprise providing a diagnosis or an assessment. In some embodiments, the diagnosis or assessment identifies the presence or absence of the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, the diagnosis or assessment identifies the individual as being predicted to have longer survival after treatment with an anti-cancer therapy, e.g., an anti-cancer therapy provided herein (e.g., an immunotherapy and/or another anti-cancer therapy provided herein), for example, as compared to an individual whose cancer does not exhibit the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the sample is a sample described herein. In some embodiments, the sample comprises cells from the cancer or is obtained from cells from the cancer, e.g., the NSCLC.

In some aspects, provided herein are methods of screening an individual having cancer, suspected of having cancer, being tested for cancer, being treated for cancer, or being tested for a susceptibility to cancer, e.g., a NSCLC. In some embodiments, the individual is being treated with an anti-cancer therapy, such as an anti-cancer therapy described herein. In some embodiments, the methods comprise acquiring knowledge of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from the individual. In some embodiments, the methods comprise detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample from the individual. In some embodiments, responsive to acquiring knowledge of a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, responsive to detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, the individual is predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the methods further comprise providing a diagnosis or an assessment. In some embodiments, the diagnosis or assessment identifies the presence or absence of the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the TMB of at least about 10 mut/Mb or at least about 20 mut/Mb, the PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the sample is a sample described herein. In some embodiments, the sample comprises cells from the cancer or is obtained from cells from the cancer, e.g., the NSCLC.

In some embodiments, the methods further comprise selectively enriching for one or more nucleic acids comprising nucleotide sequences that comprise one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 to produce an enriched sample, e.g., using a reagent known in the art or provided herein, such as an antibody described herein.

In some embodiments of any of the methods provided herein, the methods further comprise acquiring knowledge of or detecting in a sample from an individual the absence of a deletion of an MTAP gene, or of a portion thereof. In some embodiments of any of the methods provided herein, the individual does not have a deletion of an MTAP gene, or of a portion thereof.

Reporting

In some embodiments, the methods provided herein comprise generating a report, and/or providing a report to party.

In some embodiments, a report according to the present disclosure comprises information about one or more of: a TMB of at least about 10 mut/Mb or at least about 20 mut/Mb, a PD-L1-positive status, and/or one or more mutations in an RB1 gene, a KRAS gene, or a TP53 gene; a cancer of the disclosure (e.g., a NSCLC), e.g., comprising a TMB of at least about 10 mut/Mb or at least about 20 mut/Mb, a PD-L1-positive status, and/or one or more mutations in an RB1 gene, a KRAS gene, or a TP53 gene; or a treatment, a therapy, or one or more treatment options (e.g., an immunotherapy, an RB1-targeted therapy, a KRAS-targeted therapy or a TP53-targeted therapy) for an individual having a cancer, such as a cancer of the disclosure (e.g., a NSCLC, e.g., comprising a TMB of at least about 10 mut/Mb or at least about 20 mut/Mb, a PD-L1-positive status, and/or one or more mutations in an RB1 gene, a KRAS gene, or a TP53 gene). In some embodiments, a report according to the present disclosure comprises information about one or more of: a TMB of at least about 10 mut/Mb or at least about 20 mut/Mb and/or a PD-L1-positive status; a cancer of the disclosure (e.g., a NSCLC), e.g., comprising a TMB of at least about 10 mut/Mb or at least about 20 mut/Mb and/or a PD-L1-positive status; or a treatment, a therapy, or one or more treatment options (e.g., an immunotherapy) for an individual having a cancer, such as a cancer of the disclosure (e.g., a NSCLC, e.g., comprising a TMB of at least about 10 mut/Mb or at least about 20 mut/Mb and/or a PD-L1-positive status). In some embodiments, a report according to the present disclosure comprises information about one or more of: one or more mutations in an RB1 gene; a cancer of the disclosure (e.g., a NSCLC), e.g., comprising one or more mutations in an RB1 gene; or a treatment, a therapy, or one or more treatment options (e.g., an RB1-targeted therapy) for an individual having a cancer, such as a cancer of the disclosure (e.g., a NSCLC, e.g., comprising one or more mutations in an RB1 gene). In some embodiments, a report according to the present disclosure comprises information about one or more of: one or more mutations in a KRAS gene; a cancer of the disclosure (e.g., a NSCLC), e.g., comprising one or more mutations in a KRAS gene; or a treatment, a therapy, or one or more treatment options (e.g., a KRAS-targeted therapy) for an individual having a cancer, such as a cancer of the disclosure (e.g., a NSCLC, e.g., comprising one or more mutations in a KRAS gene). In some embodiments, the one or more mutations in the KRAS gene result in a G12C amino acid substitution in a KRAS polypeptide encoded by the KRAS gene. In some embodiments, a report according to the present disclosure comprises information about one or more of: one or more mutations in a TP53 gene; a cancer of the disclosure (e.g., a NSCLC), e.g., comprising one or more mutations in a TP53 gene; or a treatment, a therapy, or one or more treatment options (e.g., a TP53-targeted therapy) for an individual having a cancer, such as a cancer of the disclosure (e.g., a NSCLC, e.g., comprising one or more mutations in a TP53 gene). In some embodiments, a report according to the present disclosure further comprises information about the absence of a deletion of an MTAP gene, or of a portion thereof, in the sample. In some embodiments, a report according to the present disclosure comprises information about the presence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample.

In some embodiments, a report according to the present disclosure comprises information about the presence or absence of a TMB of at least about 10 mut/Mb or at least about 20 mut/Mb, a PD-L1-positive status, and/or one or more mutations in an RB1 gene, a KRAS gene, or a TP53 gene in a sample obtained from an individual, such as an individual having a cancer, e.g., a cancer provided herein, e.g., a NSCLC. In some embodiments, a report according to the present disclosure further comprises information about the absence of a deletion of an MTAP gene, or of a portion thereof, in the sample. In some embodiments, a report according to the present disclosure comprises information about the presence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In one embodiment, a report according to the present disclosure indicates that a TMB of at least about 10 mut/Mb or at least about 20 mut/Mb, a PD-L1-positive status, and/or one or more mutations in an RB1 gene, a KRAS gene, or a TP53 gene are present in a sample obtained from the individual. In one embodiment, a report according to the present disclosure indicates that a deletion of an MTAP gene, or of a portion thereof, is not present in the sample. In some embodiments, the one or more mutations in the KRAS gene result in a G12C amino acid substitution in a KRAS polypeptide encoded by the KRAS gene. In one embodiment, a report according to the present disclosure indicates that one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are not present in the sample. In one embodiment, a report according to the present disclosure indicates that one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 are present in the sample. In one embodiment, a report according to the present disclosure indicates that a TMB of at least about 10 mut/Mb or at least about 20 mut/Mb, a PD-L1-positive status, and/or one or more mutations in an RB1 gene, a KRAS gene, or a TP53 gene are not present in a sample obtained from the individual. In one embodiment, a report according to the present disclosure indicates that a TMB of at least about 10 mut/Mb or at least about 20 mut/Mb, a PD-L1-positive status, and/or one or more mutations in an RB1 gene, a KRAS gene, or a TP53 gene have been detected in a sample obtained from the individual. In one embodiment, a report according to the present disclosure indicates that a TMB of at least about 10 mut/Mb or at least about 20 mut/Mb, a PD-L1-positive status, and/or one or more mutations in an RB1 gene, a KRAS gene, or a TP53 gene have not been detected in a sample obtained from the individual. In some embodiments, the one or more mutations in the KRAS gene result in a G12C amino acid substitution in a KRAS polypeptide encoded by the KRAS gene. In one embodiment, a report according to the present disclosure further indicates that a deletion of an MTAP gene, or of a portion thereof, has not been detected in the sample. In one embodiment, a report according to the present disclosure indicates that one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 have not been detected in the sample. In one embodiment, a report according to the present disclosure further indicates that one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 have been detected in the sample. In some embodiments, the report comprises an identifier for the individual from which the sample was obtained.

In some embodiments, the report includes information on the role of a TMB of at least about 10 mut/Mb or at least about 20 mut/Mb, a PD-L1-positive status, and/or one or more mutations in an RB1 gene, a KRAS gene, or a TP53 gene, or of the corresponding wild type counterparts, in disease, such as in cancer, e.g., a NSCLC. In some embodiments, the report further includes information on the role of the absence of a deletion of an MTAP gene, or of a portion thereof, in disease, such as in cancer, e.g., a NSCLC. In some embodiments, the report further includes information on the role of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in disease, such as in cancer, e.g., a NSCLC. Such information can include one or more of: information on prognosis of a cancer, such as a cancer provided herein, e.g., comprising a TMB of at least about 10 mut/Mb or at least about 20 mut/Mb, a PD-L1-positive status, and/or one or more mutations in an RB1 gene, a KRAS gene, or a TP53 gene, and/or not having a deletion of an MTAP gene, or of a portion thereof; information on resistance of a cancer, such as a cancer provided herein, e.g., a NSCLC, such as a NSCLC comprising a TMB of at least about 10 mut/Mb or at least about 20 mut/Mb, a PD-L1-positive status, and/or one or more mutations in an RB1 gene, a KRAS gene, or a TP53 gene, and/or not having a deletion of an MTAP gene, or of a portion thereof, to one or more treatments; information on potential or suggested therapeutic options (e.g., such as an anti-cancer therapy provided herein, or a treatment selected or identified according to the methods provided herein, e.g., an immunotherapy, an RB1-targeted therapy, a KRAS-targeted therapy or a TP53-targeted therapy); or information on therapeutic options that should be avoided. Such information can include one or more of: information on prognosis of a cancer, such as a cancer provided herein, e.g., comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1; information on resistance of a cancer, such as a cancer provided herein, e.g., a NSCLC, such as a NSCLC comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein, e.g., an immunotherapy, an RB1-targeted therapy, a KRAS-targeted therapy or a TP53-targeted therapy) to an individual having a cancer, such as a cancer provided herein (e.g., a NSCLC), e.g., comprising a TMB of at least about 10 mut/Mb or at least about 20 mut/Mb, a PD-L1-positive status, and/or one or more mutations in an RB1 gene, a KRAS gene, or a TP53 gene, and/or not having a deletion of an MTAP gene, or of a portion thereof, and identified in the report. In some embodiments, the report includes information or a recommendation on the administration of a treatment (e.g., an anti-cancer therapy provided herein, or a treatment selected or identified according to the methods provided herein, such as an immunotherapy, an RB1-targeted therapy, a KRAS-targeted therapy or a TP53-targeted therapy). In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein (e.g., a NSCLC), e.g., comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, and identified in the report. In some embodiments, the report includes information or a recommendation on the administration of a treatment, e.g., an anti-cancer therapy provided herein, or a treatment selected or identified according to the methods provided herein, such as an immunotherapy, an RB1-targeted therapy, a KRAS-targeted therapy or a TP53-targeted therapy. In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments. In some embodiments, the one or more mutations in the KRAS gene result in a G12C amino acid substitution in a KRAS polypeptide encoded by the KRAS gene.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein (e.g., a NSCLC); detecting a TMB of at least about 10 mut/Mb or at least about 20 mut/Mb, a PD-L1-positive status, and/or one or more mutations in an RB1 gene, a KRAS gene, or a TP53 gene in the sample, or acquiring knowledge of the presence of a TMB of at least about 10 mut/Mb or at least about 20 mut/Mb, a PD-L1-positive status, and/or one or more mutations in an RB1 gene, a KRAS gene, or a TP53 gene in the sample; and generating a report. In some embodiments, the method further comprises detecting the absence of a deletion of an MTAP gene, or of a portion thereof, in the sample, or acquiring knowledge of the absence of a deletion of an MTAP gene, or of a portion thereof, in the sample. In some embodiments, the method comprises detecting one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample, or acquiring knowledge of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample. In some embodiments, a report generated according to the methods provided herein comprises one or more of: information about the presence or absence of a TMB of at least about 10 mut/Mb or at least about 20 mut/Mb, a PD-L1-positive status, and/or one or more mutations in an RB1 gene, a KRAS gene, or a TP53 gene in the sample; an identifier for the individual from which the sample was obtained; information on the role of a TMB of at least about 10 mut/Mb or at least about 20 mut/Mb, a PD-L1-positive status, and/or one or more mutations in an RB1 gene, a KRAS gene, or a TP53 gene, or the corresponding wild type counterparts, in disease (e.g., such as in cancer, e.g., a NSCLC); information on prognosis, resistance, or potential or suggested therapeutic options (such as an anti-cancer therapy provided herein, or a treatment selected or identified according to the methods provided herein, such as an immunotherapy, an RB1-targeted therapy, a KRAS-targeted therapy or a TP53-targeted therapy); information on the likely effectiveness, acceptability, or the advisability of applying a therapeutic option (such as an anti-cancer therapy provided herein, or a treatment selected or identified according to the methods provided herein, e.g., an immunotherapy, an RB1-targeted therapy, a KRAS-targeted therapy or a TP53-targeted therapy) to the individual; a recommendation or information on the administration of a treatment (such as an anti-cancer therapy provided herein, or a treatment selected or identified according to the methods provided herein, e.g., an immunotherapy, an RB1-targeted therapy, a KRAS-targeted therapy or a TP53-targeted therapy); or a recommendation or information on the dosage or treatment regimen of a treatment (such as an anti-cancer therapy provided herein, or a treatment selected or identified according to the methods provided herein, e.g., an immunotherapy, an RB1-targeted therapy, a KRAS-targeted therapy or a TP53-targeted therapy), e.g., in combination with other treatments (e.g., a second therapeutic agent). In some embodiments, the one or more mutations in the KRAS gene result in a G12C amino acid substitution in a KRAS polypeptide encoded by the KRAS gene. In some embodiments, a report generated according to the methods provided herein comprises one or more of: information about the presence or absence one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in the sample; an identifier for the individual from which the sample was obtained; information on the role of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, or the corresponding wild type counterparts, in disease (e.g., such as in cancer, e.g., a NSCLC); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, a report generated according to the methods provided herein further comprises information about the absence of a deletion of an MTAP gene, or of a portion thereof in the sample. In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., a NSCLC, such as a NSCLC comprising a TMB of at least about 10 mut/Mb or at least about 20 mut/Mb, a PD-L1-positive status, and/or one or more mutations in an RB1 gene, a KRAS gene, or a TP53 gene, and/or optionally not having a deletion of an MTAP gene, or of a portion thereof), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a TMB of at least about 10 mut/Mb or at least about 20 mut/Mb, a PD-L1-positive status, and/or one or more mutations in an RB1 gene, a KRAS gene, or a TP53 gene, and/or optionally the absence of a deletion of an MTAP gene, or of a portion thereof, in a sample obtained from an individual (e.g., an individual having a cancer, e.g., a NSCLC). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a TMB of at least about 10 mut/Mb or at least about 20 mut/Mb, a PD-L1-positive status, and/or one or more mutations in an RB1 gene, a KRAS gene, or a TP53 gene, and/or optionally of a deletion of an MTAP gene, or of a portion thereof, in a sample obtained from an individual (e.g., an individual having a cancer). In some embodiments, the one or more mutations in the KRAS gene result in a G12C amino acid substitution in a KRAS polypeptide encoded by the KRAS gene. The report may be provided to an individual or a patient (e.g., an individual or a patient with a cancer, such as a cancer provided herein, e.g., a NSCLC, such as a NSCLC comprising one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual (e.g., an individual having a cancer, e.g., a NSCLC). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 in a sample obtained from an individual (e.g., an individual having a cancer).

In some embodiments, the level of PD-L1 protein expression is determined based on PD-L1 tumor cell expression using an immunohistochemistry assay (e.g., a DAKO 22C3 assay). In some embodiments, the level of PD-L1 protein expression is assessed based on a tumor proportion score (TPS). In some embodiments, the NSCLC is determined to be PD-L1 positive if it has a PD-L1 low positive status or a PD-L1 high positive status.

Renal Cell Carcinoma

Renal cell carcinoma (RCC) is the most common type of kidney cancer in adults. Subtypes of RCC include clear cell renal cell carcinoma (ccRCC) and sarcomatoid renal cell carcinoma (srcRCC). srcRCC is an aggressive variant of RCC that frequently presents as advanced-stage disease refractory to traditional systemic treatments for clear cell renal cell carcinoma (ccRCC).

In some embodiments, a srcRCC or a ccRCC of the disclosure contains a deletion in the MTAP gene (e.g., a deletion of the entire MTAP gene, or of a portion of the MTAP gene). In some embodiments, the deletion in the MTAP gene is a deletion of a portion of thereof. In some embodiments, the deletion in the MTAP gene is a deletion of the entire MTAP gene. In some embodiments, the deletion in the MTAP gene is a heterozygous deletion. In other embodiments, the deletion in the MTAP gene is a homozygous deletion. In some embodiments, the deletion is a deletion at the 9p21 locus. In some embodiments, a srcRCC or a ccRCC of the disclosure contains a deletion of a CDKN2A gene or a CDKN2B gene. In some embodiments, the deletion of the CDKN2A gene or the CDKN2B gene is a deletion of a portion of thereof. In some embodiments, the deletion of the CDKN2A gene or the CDKN2B gene is a deletion of the entire CDKN2A gene or CDKN2B gene. In some embodiments, the deletion of the CDKN2A gene or the CDKN2B gene is a heterozygous deletion. In other embodiments, the deletion of the CDKN2A gene or the CDKN2B gene is a homozygous deletion. In some embodiments, the deletion is a deletion at the 9p21 locus. In some embodiments, a srcRCC or a ccRCC of the disclosure contains a co-deletion of an MTAP gene, or of a portion thereof, and of a CDKN2A gene and/or a CDKN2B gene, or portions thereof, e.g., at the 9p21 locus.

In some embodiments, a srcRCC or a ccRCC of the disclosure contains a deletion in the MTAP gene (e.g., a deletion of the entire MTAP gene, or of a portion of the MTAP gene) and one or more mutations in one or more genes, wherein the one or more genes comprise CDKN2A, CDKN2B, VHL, PBRM1, SETD2, BAP1, TP53, PTEN, TERT, TSC1, PIK3CA, ATM, NF2, or MET. In some embodiments, a srcRCC or a ccRCC of the disclosure contains a deletion in the MTAP gene (e.g., a deletion of the entire MTAP gene, or of a portion of the MTAP gene) and one or more mutations in the NF2 gene.

In some embodiments, a srcRCC or a ccRCC of the disclosure contains an intact MTAP gene (e.g., lacks a deletion of an MTAP gene, or of a portion thereof). In some embodiments, a srcRCC or a ccRCC of the disclosure contains an intact MTAP gene and is PD-L1-positive. In some embodiments, a ccRCC of the disclosure contains an intact MTAP gene and has a tumor mutational burden (TMB) of at least about 10 mut/Mb or at least about 20 mut/Mb. In some embodiments, a ccRCC of the disclosure contains an intact MTAP gene and one or more mutations in one or more genes, wherein the one or more genes comprise CDKN2A, CDKN2B, VHL, PBRM1, SETD2, BAP1, TP53, PTEN, TERT, TSC1, PIK3CA, ATM, NF2, or MET. In some embodiments, a ccRCC of the disclosure contains an intact MTAP gene and one or more mutations in the VHL gene. In some embodiments, a ccRCC of the disclosure contains an intact MTAP gene and one or more mutations in the PBRM1 gene.

MTAP-Deleted Renal Cell Carcinoma

In some aspects, provided herein are methods of treating or delaying progression of a sarcomatoid renal cell carcinoma (srcRCC) or a clear cell renal cell carcinoma (ccRCC) in an individual, wherein the srcRCC or the ccRCC comprises a deletion of an methylthioadenosine phosphorylase (MTAP) gene, or of a portion thereof (MTAP-deleted srcRCC or ccRCC).

In other aspects, methods are provided for identifying one or more treatment options for an individual having an MTAP-deleted srcRCC or ccRCC. In other aspects, methods are provided for selecting treatment for an individual having an MTAP-deleted srcRCC or ccRCC. In some aspects, methods are provided for predicting survival of an individual having an MTAP-deleted srcRCC or ccRCC treated with an anti-cancer therapy. In other aspects, provided herein are methods of identifying an individual having an MTAP-deleted srcRCC or ccRCC, who may benefit from a treatment comprising an anti-cancer therapy. In other aspects, provided herein are methods of selecting a therapy for an individual having an MTAP-deleted srcRCC or ccRCC. In other aspects, provided herein are methods of assessing, screening, or diagnosing an individual having an MTAP-deleted srcRCC or ccRCC.

In some embodiments, the methods comprise acquiring knowledge of or detecting in a sample from an individual having an MTAP-deleted srcRCC or ccRCC a deletion of an MTAP gene, or of a portion thereof, and one or more mutations in an NF2 gene. In some embodiments, the methods comprise acquiring knowledge of or detecting in a sample from an individual having an MTAP-deleted srcRCC or ccRCC a deletion of an MTAP gene, or of a portion thereof, and one or more mutations in one or more genes, wherein the one or more genes comprise CDKN2A, CDKN2B, VHL, PBRM1, SETD2, BAP1, TP53, PTEN, TERT, TSC1, PIK3CA, ATM, NF2, or MET.

Methods of Selecting or Identifying a Treatment

In some aspects, provided herein are methods of identifying an individual having cancer, e.g., a srcRCC or a ccRCC, who may benefit from an anti-cancer therapy, e.g., an anti-cancer therapy provided herein. In some embodiments, the methods comprise detecting a deletion of an MTAP gene, or of a portion thereof, and one or more mutations in an NF2 gene in a sample obtained from the individual. In some embodiments, the methods comprise acquiring knowledge of the presence of a deletion of an MTAP gene, or of a portion thereof, and one or more mutations in an NF2 gene in a sample obtained from the individual. In some embodiments, the presence of the deletion of an MTAP gene, or of a portion thereof, and one or more mutations in an NF2 gene in the sample identifies the individual as one who may benefit from a treatment comprising an anti-cancer therapy, e.g., an anti-cancer therapy provided herein, such as an NF2-targeted therapy. In some embodiments, detection of the deletion of an MTAP gene, or of a portion thereof, and one or more mutations in an NF2 gene in the sample identifies the individual as one who may benefit from a treatment comprising an anti-cancer therapy, e.g., an anti-cancer therapy provided herein, such as an NF2-targeted therapy. In some embodiments, responsive to knowledge of the deletion of an MTAP gene, or of a portion thereof, and one or more mutations in an NF2 gene in the sample, the individual is identified as one who may benefit from a treatment comprising an anti-cancer therapy, e.g., an anti-cancer therapy provided herein, such as an NF2-targeted therapy. In some embodiments, the presence of the deletion of an MTAP gene, or of a portion thereof, and one or more mutations in an NF2 gene in the sample identifies the individual as one who may benefit from a treatment comprising an anti-cancer therapy, e.g., an anti-cancer therapy provided herein, such as an NF2-targeted therapy. In some embodiments, detection of the deletion of an MTAP gene, or of a portion thereof, and one or more mutations in an NF2 gene in the sample identifies the individual as one who may benefit from a treatment comprising an anti-cancer therapy, e.g., an anti-cancer therapy provided herein, such as an NF2-targeted therapy. In some embodiments, responsive to knowledge of the deletion of an MTAP gene, or of a portion thereof, and one or more mutations in an NF2 gene in the sample, the individual is identified as one who may benefit from a treatment comprising an anti-cancer therapy, e.g., an anti-cancer therapy provided herein, such as an NF2-targeted therapy. In some embodiments, the deletion of an MTAP gene, or of a portion thereof, and one or more mutations in an NF2 gene are detected using any suitable method known in the art or described herein. In some embodiments, the sample is a sample described herein. In some embodiments, the sample comprises cells from the cancer or is obtained from cells from the cancer, e.g., a srcRCC or a ccRCC. In some embodiments, the presence of the deletion of an MTAP gene, or of a portion thereof, and one or more mutations in an NF2 gene in the sample identifies the individual as one who may benefit from a treatment comprising a PRMT5-targeted therapy and an additional cancer therapy, e.g., an NF2-targeted therapy. In some embodiments, detection of the deletion of an MTAP gene, or of a portion thereof, and one or more mutations in an NF2 gene in the sample identifies the individual as one who may benefit from a treatment comprising a PRMT5-targeted therapy and an additional cancer therapy, e.g., an NF2-targeted therapy. In some embodiments, responsive to knowledge of the deletion of an MTAP gene, or of a portion thereof, and one or more mutations in an NF2 gene in the sample, the individual is identified as one who may benefit from a treatment comprising a PRMT5-targeted therapy and an additional cancer therapy, e.g., an NF2-targeted therapy.

Also provided herein are methods of identifying or selecting a treatment, a therapy, or one or more treatment options for an individual having a cancer, such as a cancer described herein. In some embodiments, the cancer comprises a deletion of an MTAP gene, or of a portion thereof, and one or more mutations in an NF2 gene. In some embodiments, the methods comprise detecting the deletion of an MTAP gene, or of a portion thereof, and one or more mutations in an NF2 gene in a sample obtained from an individual having a cancer, such as a cancer described herein, e.g., a srcRCC or a ccRCC. In some embodiments, the deletion of an MTAP gene, or of a portion thereof, and one or more mutations in an NF2 gene are detected using any suitable method known in the art or described herein. In some embodiments, detection of a deletion of an MTAP gene, or of a portion thereof, and one or more mutations in an NF2 gene in a sample obtained from an individual having a cancer, e.g., a cancer described herein, e.g., a srcRCC or a ccRCC, identifies the individual as one who may benefit from an anti-cancer therapy, e.g., an anti-cancer therapy provided herein, such as an NF2-targeted therapy. In some embodiments, the presence of a deletion of an MTAP gene, or of a portion thereof, and one or more mutations in an NF2 gene in a sample obtained from an individual having a cancer, e.g., a cancer provided herein, e.g., a srcRCC or a ccRCC, identifies the individual as one who may benefit from an anti-cancer therapy, such as an anti-cancer therapy provided herein, e.g., an NF2-targeted therapy. In some embodiments, responsive to detection of a deletion of an MTAP gene, or of a portion thereof, and one or more mutations in an NF2 gene in a sample obtained from an individual having a cancer, e.g., a cancer provided herein, e.g., a srcRCC or a ccRCC, the individual is classified as a candidate to receive an anti-cancer therapy, such as an anti-cancer therapy provided herein, e.g., an NF2-targeted therapy. In some embodiments, responsive to detection of a deletion of an MTAP gene, or of a portion thereof, and one or more mutations in an NF2 gene in a sample obtained from an individual having a cancer, e.g., a cancer provided herein, e.g., a srcRCC or a ccRCC, the individual is classified or identified as likely to respond to an anti-cancer therapy, such as an anti-cancer therapy provided herein, e.g., an NF2-targeted therapy. In some embodiments, the sample is a sample described herein. In some embodiments, detection of a deletion of an MTAP gene, or of a portion thereof, and one or more mutations in an NF2 gene in a sample obtained from an individual having a cancer, e.g., a cancer described herein, e.g., a srcRCC or a ccRCC, identifies the individual as one who may benefit from an anti-cancer therapy, e.g., an anti-cancer therapy provided herein, such as an NF2-targeted therapy. In some embodiments, the presence of a deletion of an MTAP gene, or of a portion thereof, and one or more mutations in an NF2 gene in a sample obtained from an individual having a cancer, e.g., a cancer provided herein, e.g., a srcRCC or a ccRCC, identifies the individual as one who may benefit from an anti-cancer therapy, such as an anti-cancer therapy provided herein, e.g., an NF2-targeted therapy. In some embodiments, responsive to detection of a deletion of an MTAP gene, or of a portion thereof, and one or more mutations in an NF2 gene in a sample obtained from an individual having a cancer, e.g., a cancer provided herein, e.g., a srcRCC or a ccRCC, the individual is classified as a candidate to receive an anti-cancer therapy, such as an anti-cancer therapy provided herein, e.g., an NF2-targeted therapy. In some embodiments, responsive to detection of a deletion of an MTAP gene, or of a portion thereof, and one or more mutations in an NF2 gene in a sample obtained from an individual having a cancer, e.g., a cancer provided herein, e.g., a srcRCC or a ccRCC, the individual is classified or identified as likely to respond to an anti-cancer therapy, such as an anti-cancer therapy provided herein, e.g., an NF2-targeted therapy. In some embodiments, the sample comprises cells from the cancer or is obtained from cells from the cancer, e.g., a srcRCC or a ccRCC. In some embodiments, the methods further comprise generating a report, e.g., as described herein. In some embodiments, the report comprises a treatment, a therapy, or one or more treatment options identified or selected for the individual, e.g., based at least in part on detection of a deletion of an MTAP gene, or of a portion thereof, and one or more mutations in an NF2 gene in the sample. In some embodiments, detection of a deletion of an MTAP gene, or of a portion thereof, and one or more mutations in an NF2 gene in a sample obtained from an individual having a cancer, e.g., a cancer described herein, e.g., a srcRCC or a ccRCC, identifies the individual as one who may benefit from a PRMT5-targeted therapy and an additional anti-cancer therapy, e.g., an NF2-targeted therapy. In some embodiments, the presence of a deletion of an MTAP gene, or of a portion thereof, and one or more mutations in an NF2 gene in a sample obtained from an individual having a cancer, e.g., a cancer provided herein, e.g., a srcRCC or a ccRCC, identifies the individual as one who may benefit from a PRMT5-targeted therapy and an additional anti-cancer therapy, e.g., an NF2-targeted therapy. In some embodiments, responsive to detection of a deletion of an MTAP gene, or of a portion thereof, and one or more mutations in an NF2 gene in a sample obtained from an individual having a cancer, e.g., a cancer provided herein, e.g., a srcRCC or a ccRCC, the individual is classified as a candidate to receive a PRMT5-targeted therapy and an additional anti-cancer therapy, e.g., an NF2-targeted therapy. In some embodiments, responsive to detection of a deletion of an MTAP gene, or of a portion thereof, and one or more mutations in an NF2 gene in a sample obtained from an individual having a cancer, e.g., a cancer provided herein, e.g., a srcRCC or a ccRCC, the individual is classified or identified as likely to respond to a PRMT5-targeted therapy and an additional anti-cancer therapy, e.g., an NF2-targeted therapy.

In some aspects, provided herein are methods of identifying an individual having cancer, e.g., a srcRCC or a ccRCC, who may benefit from an anti-cancer therapy, e.g., an anti-cancer therapy provided herein. In some embodiments, the methods comprise detecting a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, the methods comprise acquiring knowledge of the presence of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, the presence of the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the individual as one who may benefit from a treatment comprising an anti-cancer therapy, e.g., an anti-cancer therapy provided herein. In some embodiments, detection of the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the individual as one who may benefit from a treatment comprising an anti-cancer therapy, e.g., an anti-cancer therapy provided herein. In some embodiments, responsive to knowledge of the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is identified as one who may benefit from a treatment comprising an anti-cancer therapy, e.g., an anti-cancer therapy provided herein. In some embodiments, the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET are detected using any suitable method known in the art or described herein. In some embodiments, the sample is a sample described herein. In some embodiments, the sample comprises cells from the cancer or is obtained from cells from the cancer, e.g., a srcRCC or a ccRCC. In some embodiments, the presence of the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the individual as one who may benefit from a treatment comprising a PRMT5-targeted therapy and an additional cancer therapy, e.g., an anti-cancer therapy provided herein. In some embodiments, detection of the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the individual as one who may benefit from a treatment comprising a PRMT5-targeted therapy and an additional cancer therapy, e.g., an anti-cancer therapy provided herein. In some embodiments, responsive to knowledge of the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is identified as one who may benefit from a treatment comprising a PRMT5-targeted therapy and an additional cancer therapy, e.g., an anti-cancer therapy provided herein.

Also provided herein are methods of identifying or selecting a treatment, a therapy, or one or more treatment options for an individual having a cancer, such as a cancer described herein, e.g., a srcRCC or a ccRCC. In some embodiments, the cancer comprises a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the methods comprise detecting the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, such as a cancer described herein, e.g., a srcRCC or a ccRCC. In some embodiments, the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET are detected using any suitable method known in the art or described herein. In some embodiments, detection of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer described herein, e.g., a srcRCC or a ccRCC, identifies the individual as one who may benefit from an anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a srcRCC or a ccRCC, identifies the individual as one who may benefit from an anti-cancer therapy, such as an anti-cancer therapy provided herein. In some embodiments, responsive to detection of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a srcRCC or a ccRCC, the individual is classified as a candidate to receive an anti-cancer therapy, such as an anti-cancer therapy provided herein. In some embodiments, responsive to detection of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a srcRCC or a ccRCC, the individual is classified or identified as likely to respond to an anti-cancer therapy, such as an anti-cancer therapy provided herein. In some embodiments, the sample is a sample described herein. In some embodiments, the sample comprises cells from the cancer or is obtained from cells from the cancer, e.g., a srcRCC or a ccRCC. In some embodiments, the methods further comprise generating a report, e.g., as described herein. In some embodiments, the report comprises a treatment, a therapy, or one or more treatment options identified or selected for the individual, e.g., based at least in part on detection of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, detection of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer described herein, e.g., a srcRCC or a ccRCC, identifies the individual as one who may benefit from a PRMT5-targeted therapy and an additional anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a srcRCC or a ccRCC, identifies the individual as one who may benefit from a PRMT5-targeted therapy and an additional anti-cancer therapy, e.g., an anti-cancer therapy provided herein. In some embodiments, responsive to detection of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a srcRCC or a ccRCC, the individual is classified as a candidate to receive a PRMT5-targeted therapy and an additional anti-cancer therapy, e.g., an anti-cancer therapy provided herein. In some embodiments, responsive to detection of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a srcRCC or a ccRCC, the individual is classified or identified as likely to respond to a PRMT5-targeted therapy and an additional anti-cancer therapy, e.g., an anti-cancer therapy provided herein.

In some embodiments of any of the methods provided herein, the methods further comprise acquiring knowledge of or detecting in a sample from an individual a deletion of a CDKN2A gene, or of a portion thereof; and/or a deletion of a CDKN2B gene, or of a portion thereof. In some embodiments of any of the methods provided herein, the individual has a deletion of a CDKN2A gene, or of a portion thereof; and/or a deletion of a CDKN2B gene, or of a portion thereof. In some embodiments of any of the methods provided herein, the individual has a heterozygous or a homozygous deletion of a CDKN2A gene, or of a portion thereof; and/or a heterozygous or a homozygous deletion of a CDKN2B gene, or of a portion thereof. In some embodiments of any of the methods provided herein, the individual has a heterozygous or a homozygous deletion of the MTAP gene, or of a portion thereof.

### Methods of Diagnosing, Assessing, Screening, Monitoring or Predicting

In some aspects, provided herein are methods of diagnosing or assessing a deletion of an MTAP gene, or of a portion thereof, and one or more mutations in an NF2 gene in a sample from an individual having a cancer, such as a srcRCC or a ccRCC. In some embodiments, the methods comprise acquiring knowledge of the presence of a deletion of an MTAP gene, or of a portion thereof, and one or more mutations in an NF2 gene in a sample obtained from the individual. In some embodiments, the methods comprise detecting a deletion of an MTAP gene, or of a portion thereof, and one or more mutations in an NF2 gene in a sample obtained from the individual. In some embodiments, a deletion of an MTAP gene, or of a portion thereof, and one or more mutations in an NF2 gene are detected in a sample obtained from the individual using any method known in the art, such as one or more of the methods of detection of nucleic acid molecules or polypeptides described herein. In some embodiments, the methods further comprise providing a diagnosis or an assessment of the deletion of the MTAP gene, or of the portion thereof, and one or more mutations in an NF2 gene. In some embodiments, the diagnosis or assessment identifies the presence or absence of a deletion of an MTAP gene, or of a portion thereof, and one or more mutations in an NF2 gene in the sample. In some embodiments, the diagnosis or assessment identifies the cancer, such as a srcRCC or a ccRCC provided herein, as likely to respond to an anti-cancer therapy, e.g., an anti-cancer therapy provided herein, such as an NF2-targeted therapy. In some embodiments, the presence of a deletion of an MTAP gene, or of a portion thereof, and one or more mutations in an NF2 gene in the sample identifies the cancer as likely to respond to an anti-cancer therapy, e.g., an anti-cancer therapy provided herein, such as an NF2-targeted therapy. In some embodiments, the diagnosis or assessment identifies the presence or absence of a deletion of an MTAP gene, or of a portion thereof, and one or more mutations in an NF2 gene in the sample. In some embodiments, the diagnosis or assessment identifies the cancer, such as a srcRCC or a ccRCC provided herein, as likely to respond to an anti-cancer therapy, e.g., an anti-cancer therapy provided herein, such as an NF2-targeted therapy. In some embodiments, the presence of a deletion of an MTAP gene, or of a portion thereof, and one or more mutations in an NF2 gene in the sample identifies the cancer as likely to respond to an anti-cancer therapy, e.g., an anti-cancer therapy provided herein, such as an NF2-targeted therapy. In some embodiments, the sample is a sample described herein. In some embodiments, the sample comprises cells from the cancer or is obtained from cells from the cancer, e.g., from a srcRCC or a ccRCC. In some embodiments, the individual has a cancer, is suspected of having a cancer, is being tested for a cancer, is being treated for a cancer, or is being tested for a susceptibility to a cancer, e.g., a srcRCC or a ccRCC. In some embodiments, the diagnosis or assessment identifies the cancer, such as a srcRCC or ccRCC provided herein, as likely to respond to a PRMT5-targeted therapy and an additional anti-cancer therapy, e.g., an NF2-targeted therapy. In some embodiments, the presence of a deletion of an MTAP gene, or of a portion thereof, and one or more mutations in an NF2 gene in the sample identifies the cancer as likely to respond to a PRMT5-targeted therapy and an additional anti-cancer therapy, e.g., an NF2-targeted therapy.

In some aspects, provided herein are methods of diagnosing or assessing a cancer in an individual, e.g., a srcRCC or a ccRCC. In some embodiments, the methods of diagnosing or assessing cancer comprise detecting a deletion of an MTAP gene, or of a portion thereof, and one or more mutations in an NF2 gene in a sample obtained from the individual, e.g., a sample comprising cells from the cancer, e.g., a srcRCC or a ccRCC. In some embodiments, the methods comprise detecting a deletion of an MTAP gene, or of a portion thereof, and one or more mutations in an NF2 gene in a sample obtained from the individual using any method known in the art, such as one or more of the methods of detection of nucleic acid molecules or polypeptides described herein. In some embodiments, detection of a deletion of an MTAP gene, or of a portion thereof, and one or more mutations in an NF2 gene in a sample obtained from the individual identifies the cancer, e.g., the srcRCC or the ccRCC, as likely to respond to an anti-cancer therapy, e.g., an anti-cancer therapy provided herein, such as an NF2-targeted therapy. In some embodiments, the presence of a deletion of an MTAP gene, or of a portion thereof, and one or more mutations in an NF2 gene in a sample obtained from the individual identifies the cancer, e.g., the srcRCC or the ccRCC, as likely to respond to an anti-cancer therapy, e.g., an anti-cancer therapy provided herein, such as an NF2-targeted therapy. In some embodiments, the methods further comprise providing a diagnosis or an assessment of the cancer, e.g., the srcRCC or the ccRCC, or of the deletion of the MTAP gene, or of a portion thereof, and one or more mutations in an NF2 gene. In some embodiments, the diagnosis or assessment identifies the cancer as likely to respond to an anti-cancer therapy, e.g., an anti-cancer therapy provided herein, such as an NF2-targeted therapy. In some embodiments, detection of a deletion of an MTAP gene, or of a portion thereof, and one or more mutations in an NF2 gene in a sample obtained from the individual identifies the cancer, e.g., the srcRCC or the ccRCC, as likely to respond to an anti-cancer therapy, e.g., an anti-cancer therapy provided herein, such as an NF2-targeted therapy. In some embodiments, the presence of a deletion of an MTAP gene, or of a portion thereof, and one or more mutations in an NF2 gene in a sample obtained from the individual identifies the cancer, e.g., the srcRCC or the ccRCC, as likely to respond to an anti-cancer therapy, e.g., an anti-cancer therapy provided herein, such as an NF2-targeted therapy. In some embodiments, the methods further comprise providing a diagnosis or an assessment of the cancer, e.g., the srcRCC or the ccRCC, or of the deletion of the MTAP gene, or of a portion thereof, and one or more mutations in an NF2 gene. In some embodiments, the diagnosis or assessment identifies the cancer as likely to respond to an anti-cancer therapy, e.g., an anti-cancer therapy provided herein, such as an NF2-targeted therapy. In some embodiments, the diagnosis or assessment identifies the presence or absence of the deletion of the MTAP gene, or of a portion thereof, and one or more mutations in an NF2 gene in the sample. In some embodiments, detection of a deletion of an MTAP gene, or of a portion thereof, and one or more mutations in an NF2 gene in a sample obtained from the individual identifies the cancer, e.g., the srcRCC or ccRCC, as likely to respond to a PRMT5-targeted therapy and an additional anti-cancer therapy, e.g., an NF2-targeted therapy. In some embodiments, the presence of a deletion of an MTAP gene, or of a portion thereof, and one or more mutations in an NF2 gene in a sample obtained from the individual identifies the cancer, e.g., the srcRCC or ccRCC, as likely to respond to a PRMT5-targeted therapy and an additional anti-cancer therapy, e.g., an NF2-targeted therapy. In some embodiments, the diagnosis or assessment identifies the cancer as likely to respond to a PRMT5-targeted therapy and an additional anti-cancer therapy, e.g., an NF2-targeted therapy.

In some aspects, provided herein are methods of predicting survival of an individual having a cancer, e.g., a cancer provided herein, e.g., a srcRCC or a ccRCC. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in an NF2 gene in a sample from the individual. In some embodiments, the methods comprise detecting a deletion of an MTAP gene, or of a portion thereof, and one or more mutations in an NF2 gene in a sample from the individual. In some embodiments, responsive to acquiring knowledge of a deletion of an MTAP gene, or of a portion thereof, and one or more mutations in an NF2 gene in the sample, the individual is predicted to have longer survival after treatment with an anti-cancer therapy, e.g., an anti-cancer therapy provided herein (e.g., an NF2-targeted therapy), for example, as compared to an individual whose cancer does not exhibit the deletion of the MTAP gene, or of a portion thereof, and one or more mutations in an NF2 gene. In some embodiments, responsive to detecting a deletion of an MTAP gene, or of a portion thereof, and one or more mutations in an NF2 gene in the sample, the individual is predicted to have longer survival after treatment with an anti-cancer therapy, e.g., an anti-cancer therapy provided herein (e.g., an NF2-targeted therapy), for example, as compared to an individual whose cancer does not exhibit the deletion of the MTAP gene, or of a portion thereof, and one or more mutations in an NF2 gene. In some embodiments, responsive to acquiring knowledge of a deletion of an MTAP gene, or of a portion thereof, and one or more mutations in an NF2 gene in the sample, the individual is predicted to have longer survival after treatment with an anti-cancer therapy, e.g., an anti-cancer therapy provided herein (e.g., an NF2-targeted therapy), for example, as compared to an individual whose cancer does not exhibit the deletion of the MTAP gene, or of a portion thereof, and one or more mutations in an NF2 gene. In some embodiments, responsive to detecting a deletion of an MTAP gene, or of a portion thereof, and one or more mutations in an NF2 gene in the sample, the individual is predicted to have longer survival after treatment with an anti-cancer therapy, e.g., an anti-cancer therapy provided herein (e.g., an NF2-targeted therapy), for example, as compared to an individual whose cancer does not exhibit the deletion of the MTAP gene, or of a portion thereof, and one or more mutations in an NF2 gene. In some embodiments, the methods further comprise providing a diagnosis or an assessment. In some embodiments, the diagnosis or assessment identifies the presence or absence of the deletion of an MTAP gene, or of a portion thereof, and one or more mutations in an NF2 gene in the sample. In some embodiments, the diagnosis or assessment identifies the individual as being predicted to have longer survival after treatment with an anti-cancer therapy, e.g., an anti-cancer therapy provided herein (e.g., an NF2-targeted therapy), for example, as compared to an individual whose cancer does not exhibit the deletion of the MTAP gene, or of a portion thereof, and one or more mutations in an NF2 gene. In some embodiments, the diagnosis or assessment identifies the individual as being predicted to have longer survival after treatment with an anti-cancer therapy, e.g., an anti-cancer therapy provided herein (e.g., an NF2-targeted therapy), for example, as compared to an individual whose cancer does not exhibit the deletion of the MTAP gene, or of a portion thereof, and one or more mutations in an NF2 gene. In some embodiments, the sample is a sample described herein. In some embodiments, the sample comprises cells from the cancer or is obtained from cells from the cancer, e.g., the srcRCC or the ccRCC. In some embodiments, responsive to acquiring knowledge of a deletion of an MTAP gene, or of a portion thereof, and one or more mutations in an NF2 gene in the sample, the individual is predicted to have longer survival after treatment with a PRMT5-targeted therapy and an additional anti-cancer therapy, e.g., an NF2-targeted therapy, for example, as compared to an individual whose cancer does not exhibit the deletion of the MTAP gene, or of a portion thereof, and one or more mutations in an NF2 gene. In some embodiments, responsive to detecting a deletion of an MTAP gene, or of a portion thereof, and one or more mutations in an NF2 gene in the sample, the individual is predicted to have longer survival after treatment with a PRMT5-targeted therapy and an additional anti-cancer therapy, e.g., an NF2-targeted therapy, for example, as compared to an individual whose cancer does not exhibit the deletion of the MTAP gene, or of a portion thereof, and one or more mutations in an NF2 gene. In some embodiments, the diagnosis or assessment identifies the individual as being predicted to have longer survival after treatment with a PRMT5-targeted therapy and an additional anti-cancer therapy, e.g., an NF2-targeted therapy, for example, as compared to an individual whose cancer does not exhibit the deletion of the MTAP gene, or of a portion thereof, and one or more mutations in an NF2 gene.

In some aspects, provided herein are methods of screening an individual having cancer, suspected of having cancer, being tested for cancer, being treated for cancer, or being tested for a susceptibility to cancer, e.g., a srcRCC or a ccRCC. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in an NF2 gene in a sample from the individual. In some embodiments, the methods comprise detecting a deletion of an MTAP gene, or of a portion thereof, and one or more mutations in an NF2 gene in a sample from the individual. In some embodiments, responsive to acquiring knowledge of a deletion of an MTAP gene, or of a portion thereof, and one or more mutations in an NF2 gene in the sample, the individual is predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in an NF2 gene. In some embodiments, responsive to detecting a deletion of an MTAP gene, or of a portion thereof, and one or more mutations in an NF2 gene in the sample, the individual is predicted to have increased risk of cancer recurrence, aggressive cancer, anti-cancer therapy resistance, or poor prognosis, for example, as compared to an individual whose cancer does not exhibit the deletion of an MTAP gene, or of a portion thereof, and one or more mutations in an NF2 gene. In some embodiments, the methods further comprise providing a diagnosis or an assessment. In some embodiments, the diagnosis or assessment identifies the presence or absence of the deletion of the MTAP gene, or of a portion thereof, and one or more mutations in an NF2 gene in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in an NF2 gene. In some embodiments, the sample is a sample described herein. In some embodiments, the sample comprises cells from the cancer or is obtained from cells from the cancer, e.g., the srcRCC or the ccRCC.

In some embodiments, the methods further comprise selectively enriching for one or more nucleic acids comprising nucleotide sequences that comprise one or more mutations in an NF2 gene to produce an enriched sample, e.g., using a reagent known in the art or provided herein, such as a bait, probe, or oligonucleotide described herein. In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample. In some embodiments, the methods further comprise selectively enriching for one or more polypeptides comprising amino acid sequences that comprise one or more mutations in an NF2 gene to produce an enriched sample, e.g., using a reagent known in the art or provided herein, such as an antibody described herein. In some embodiments, the methods further comprise selectively enriching for one or more nucleic acids comprising nucleotide sequences that comprise a deletion of an MTAP gene, or of a portion thereof, to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise a deletion of an MTAP gene, or of a portion thereof, to produce an enriched sample, e.g., using a reagent known in the art or provided herein, such as an antibody described herein.

In some aspects, provided herein are methods of diagnosing or assessing a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from an individual having a cancer, such as a srcRCC or a ccRCC. In some embodiments, the methods comprise acquiring knowledge of the presence of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, the methods comprise detecting a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET are detected in a sample obtained from the individual using any method known in the art, such as one or more of the methods of detection of nucleic acid molecules or polypeptides described herein. In some embodiments, the methods further comprise providing a diagnosis or an assessment of the deletion of the MTAP gene, or of the portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the diagnosis or assessment identifies the presence or absence of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, the diagnosis or assessment identifies the cancer, such as a srcRCC or a ccRCC provided herein, as likely to respond to an anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the cancer as likely to respond to an anti-cancer therapy, e.g., an anti-cancer therapy provided herein. In some embodiments, the sample is a sample described herein. In some embodiments, the sample comprises cells from the cancer or is obtained from cells from the cancer, e.g., from a srcRCC or a ccRCC. In some embodiments, the individual has a cancer, is suspected of having a cancer, is being tested for a cancer, is being treated for a cancer, or is being tested for a susceptibility to a cancer, e.g., a srcRCC or a ccRCC. In some embodiments, the diagnosis or assessment identifies the cancer, such as a srcRCC or ccRCC provided herein, as likely to respond to a PRMT5-targeted therapy and an additional anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the cancer as likely to respond to a PRMT5-targeted therapy and an additional anti-cancer therapy, e.g., an anti-cancer therapy provided herein.

In some aspects, provided herein are methods of diagnosing or assessing a cancer in an individual, e.g., a srcRCC or a ccRCC. In some embodiments, the methods of diagnosing or assessing cancer comprise detecting a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual, e.g., a sample comprising cells from the cancer, e.g., a srcRCC or a ccRCC. In some embodiments, the methods comprise detecting a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual using any method known in the art, such as one or more of the methods of detection of nucleic acid molecules or polypeptides described herein. In some embodiments, detection of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual identifies the cancer, e.g., the srcRCC or the ccRCC, as likely to respond to an anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual identifies the cancer, e.g., the srcRCC or the ccRCC, as likely to respond to an anti-cancer therapy, e.g., an anti-cancer therapy provided herein. In some embodiments, the methods further comprise providing a diagnosis or an assessment of the cancer, e.g., the srcRCC or the ccRCC, or of the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the diagnosis or assessment identifies the cancer as likely to respond to an anti-cancer therapy, e.g., an anti-cancer therapy provided herein. In some embodiments, the diagnosis or assessment identifies the presence or absence of the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, detection of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual identifies the cancer, e.g., the srcRCC or ccRCC, as likely to respond to a PRMT5-targeted therapy and an additional anti-cancer therapy, e.g., an anti-cancer therapy provided herein. In some embodiments, the presence of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual identifies the cancer, e.g., the srcRCC or ccRCC, as likely to respond to a PRMT5-targeted therapy and an additional anti-cancer therapy, e.g., an anti-cancer therapy provided herein. In some embodiments, the diagnosis or assessment identifies the cancer as likely to respond to a PRMT5-targeted therapy and an additional anti-cancer therapy, e.g., an anti-cancer therapy provided herein.

In some aspects, provided herein are methods of predicting survival of an individual having a cancer, e.g., a cancer provided herein, e.g., a srcRCC or a ccRCC. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from the individual. In some embodiments, the methods comprise detecting a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from the individual. In some embodiments, responsive to acquiring knowledge of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have longer survival after treatment with an anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, responsive to detecting a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have longer survival after treatment with an anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the methods further comprise providing a diagnosis or an assessment. In some embodiments, the diagnosis or assessment identifies the presence or absence of the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the sample is a sample described herein. In some embodiments, the sample comprises cells from the cancer or is obtained from cells from the cancer, e.g., the srcRCC or the ccRCC. In some embodiments, responsive to acquiring knowledge of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have longer survival after treatment with a PRMT5-targeted therapy and an additional anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, responsive to detecting a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have longer survival after treatment with a PRMT5-targeted therapy and an additional anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the diagnosis or assessment identifies the individual as being predicted to have longer survival after treatment with a PRMT5-targeted therapy and an additional anti-cancer therapy, e.g., an anti-cancer therapy provided herein, for example, as compared to an individual whose cancer does not exhibit the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET.

In some aspects, provided herein are methods of screening an individual having cancer, suspected of having cancer, being tested for cancer, being treated for cancer, or being tested for a susceptibility to cancer, e.g., a srcRCC or a ccRCC. In some embodiments, the individual is being treated with an anti-cancer therapy, such as an anti-cancer therapy described herein. In some embodiments, the individual is being treated with a PRMT5-targeted therapy. In some embodiments, the methods comprise acquiring knowledge of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from the individual. In some embodiments, the methods comprise detecting a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from the individual. In some embodiments, responsive to acquiring knowledge of a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, responsive to detecting a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have increased risk of cancer recurrence, aggressive cancer, anti-cancer therapy resistance, or poor prognosis, for example, as compared to an individual whose cancer does not exhibit the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the methods further comprise providing a diagnosis or an assessment. In some embodiments, the diagnosis or assessment identifies the presence or absence of the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the deletion of the MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the sample is a sample described herein. In some embodiments, the sample comprises cells from the cancer or is obtained from cells from the cancer, e.g., the srcRCC or the ccRCC.

In some embodiments, the methods further comprise selectively enriching for one or more nucleic acids comprising nucleotide sequences that comprise one or more mutations in one or more genes selected from CDKN2A, CDKN2B, VHL, PBRM1, SETD2, BAP1, TP53, PTEN, TERT, TSC1, PIK3CA, ATM, NF2, or MET to produce an enriched sample, e.g., using a reagent known in the art or provided herein, such as a bait, probe, or oligonucleotide described herein. In some embodiments, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the selectively enriching comprises amplifying the one or more nucleic acids in the sample using a polymerase chain reaction (PCR) to produce the enriched sample. In some embodiments, the methods further comprise sequencing the one or more nucleic acid molecules in the enriched sample. In some embodiments, the methods further comprise selectively enriching for one or more polypeptides comprising amino acid sequences that comprise one or more mutations in one or more genes selected from CDKN2A, CDKN2B, VHL, PBRM1, SETD2, BAP1, TP53, PTEN, TERT, TSC1, PIK3CA, ATM, NF2, or MET to produce an enriched sample, e.g., using a reagent known in the art or provided herein, such as an antibody described herein. In some embodiments, the methods further comprise selectively enriching for one or more nucleic acids comprising nucleotide sequences that comprise a deletion of an MTAP gene, or of a portion thereof, to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise a deletion of an MTAP gene, or of a portion thereof, to produce an enriched sample, e.g., using a reagent known in the art or provided herein, such as an antibody described herein.

In some embodiments of any of the methods provided herein, the methods further comprise acquiring knowledge of or detecting in a sample from an individual a deletion of a CDKN2A gene, or of a portion thereof; and/or a deletion of a CDKN2B gene, or of a portion thereof. In some embodiments of any of the methods provided herein, the individual has a deletion of a CDKN2A gene, or of a portion thereof; and/or a deletion of a CDKN2B gene, or of a portion thereof. In some embodiments of any of the methods provided herein, the individual has a heterozygous or a homozygous deletion of a CDKN2A gene, or of a portion thereof; and/or a heterozygous or a homozygous deletion of a CDKN2B gene, or of a portion thereof. In some embodiments of any of the methods provided herein, the individual has a heterozygous or a homozygous deletion of the MTAP gene, or of a portion thereof.

Methods of Treatment

Also provided herein are methods of treating or delaying progression of a cancer in an individual, such as a cancer provided herein, e.g., a srcRCC or a ccRCC. In some embodiments, the individual has a cancer comprising a deletion of an MTAP gene, or of a portion thereof, and one or more mutations in an NF2 gene. In some embodiments, the methods of treating or delaying progression of a cancer of the disclosure, e.g., a srcRCC or a ccRCC comprising a deletion of an MTAP gene, or of a portion thereof, and one or more mutations in an NF2 gene, comprise administering to the individual a therapeutically effective amount of an anti-cancer therapy, such as an anti-cancer therapy provided herein, e.g., an NF2-targeted therapy. In some embodiments, the methods of treating or delaying progression of a cancer of the disclosure, e.g., a srcRCC or a ccRCC comprising a deletion of an MTAP gene, or of a portion thereof, and one or more mutations in an NF2 gene, comprise administering to the individual an effective amount of an anti-cancer therapy, such as an anti-cancer therapy provided herein (e.g., an NF2-targeted therapy), responsive to knowledge of the presence of the deletion of an MTAP gene, or of a portion thereof, and one or more mutations in an NF2 gene in a sample obtained from the individual. In some embodiments, the sample is a sample described herein. In some embodiments, the sample comprises cells from the cancer or is obtained from cells from the cancer. The methods of treatment disclosed herein may include any of the anti-cancer therapies and/or therapeutic agents described herein, e.g., infra.

In some embodiments, the individual has a cancer comprising a deletion of an MTAP gene, or of a portion thereof, and one or more mutations in an NF2 gene. In some embodiments, the methods of treating or delaying progression of a cancer of the disclosure, e.g., a srcRCC or a ccRCC comprising a deletion of an MTAP gene, or of a portion thereof, and one or more mutations in an NF2 gene, comprise administering to the individual a therapeutically effective amount of an anti-cancer therapy, such as an anti-cancer therapy provided herein, e.g., an NF2-targeted therapy. In some embodiments, the methods of treating or delaying progression of a cancer of the disclosure, e.g., a srcRCC or a ccRCC comprising a deletion of an MTAP gene, or of a portion thereof, and one or more mutations in an NF2 gene, comprise administering to the individual an effective amount of an anti-cancer therapy, such as an anti-cancer therapy provided herein (e.g., an NF2-targeted therapy), responsive to knowledge of the presence of the deletion of an MTAP gene, or of a portion thereof, and one or more mutations in an NF2 gene in a sample obtained from the individual. In some embodiments, the NF2-targeted therapy comprises one or more of an mTOR inhibitor, a VEGF inhibitor, a focal adhesion kinase (FAK) inhibitor, an EGFR inhibitor, a NEDD8-activating enzyme (NAE) inhibitor, a MET inhibitor, a MEK inhibitor, a SRC inhibitor, a JNK inhibitor, a CDK inhibitor, a WEE1, a CHK1 inhibitor, or a multi-targeted kinase inhibitor.

Also provided herein are methods of treating or delaying progression of a cancer in an individual, such as a cancer provided herein, e.g., a srcRCC or a ccRCC. In some embodiments, the individual has a cancer comprising a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the methods of treating or delaying progression of a cancer of the disclosure, e.g., a srcRCC or a ccRCC comprising a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET, comprise administering to the individual a therapeutically effective amount of an anti-cancer therapy, such as an anti-cancer therapy provided herein. In some embodiments, the methods of treating or delaying progression of a cancer of the disclosure, e.g., a srcRCC or a ccRCC comprising a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET, comprise administering to the individual an effective amount of an anti-cancer therapy, such as an anti-cancer therapy provided herein, responsive to knowledge of the presence of the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, the sample is a sample described herein. In some embodiments, the sample comprises cells from the cancer or is obtained from cells from the cancer. The methods of treatment disclosed herein may include any of the anti-cancer therapies and/or therapeutic agents described herein, e.g., infra.

In some embodiments, the methods of treating or delaying progression of a cancer of the disclosure, e.g., a srcRCC or ccRCC comprising a deletion of an MTAP gene, or of a portion thereof, and one or more mutations in an NF2 gene, comprise administering to the individual a therapeutically effective amount of a PRMT5-targeted therapy and an additional anti-cancer therapy, e.g., an NF2-targeted therapy. In some embodiments, the methods of treating or delaying progression of a cancer of the disclosure, e.g., a srcRCC or ccRCC comprising a deletion of an MTAP gene, or of a portion thereof, and one or more mutations in an NF2 gene, comprise administering to the individual an effective amount of a PRMT5-targeted therapy and an additional anti-cancer therapy, e.g., an NF2-targeted therapy, responsive to knowledge of the presence of the deletion of an MTAP gene, or of a portion thereof, and one or more mutations in an NF2 gene in a sample obtained from the individual.

In some embodiments, the methods of treating or delaying progression of a cancer of the disclosure, e.g., a srcRCC or ccRCC comprising a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET, comprise administering to the individual a therapeutically effective amount of a PRMT5-targeted therapy and an additional anti-cancer therapy, e.g., an anti-cancer therapy provided herein. In some embodiments, the methods of treating or delaying progression of a cancer of the disclosure, e.g., a srcRCC or ccRCC comprising a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET, comprise administering to the individual an effective amount of a PRMT5-targeted therapy and an additional anti-cancer therapy, e.g., an anti-cancer therapy, responsive to knowledge of the presence of the deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual.

In some embodiments, the anti-cancer therapy is administered in combination with a second anti-cancer therapy or agent, such as any of the anti-cancer agents or therapies described herein. In some embodiments, the second anti-cancer therapy or agent comprises a PRMT5-targeted therapy. In some embodiments, the anti-cancer therapy comprises any of the anti-cancer therapies provided herein and a PRMT5-targeted therapy. In some embodiments, the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

In some embodiments of any of the methods provided herein, the methods further comprise acquiring knowledge of or detecting in a sample from an individual a deletion of a CDKN2A gene, or of a portion thereof; and/or a deletion of a CDKN2B gene, or of a portion thereof. In some embodiments of any of the methods provided herein, the individual has a deletion of a CDKN2A gene, or of a portion thereof; and/or a deletion of a CDKN2B gene, or of a portion thereof. In some embodiments of any of the methods provided herein, the individual has a heterozygous or a homozygous deletion of a CDKN2A gene, or of a portion thereof; and/or a heterozygous or a homozygous deletion of a CDKN2B gene, or of a portion thereof. In some embodiments of any of the methods provided herein, the individual has a heterozygous or a homozygous deletion of the MTAP gene, or of a portion thereof.

Reporting

In some embodiments, the methods provided herein comprise generating a report, and/or providing a report to party.

In some embodiments, a report according to the present disclosure comprises information about one or more of: a deletion of an MTAP gene, or of a portion thereof, and one or more mutations in an NF2 gene; a cancer of the disclosure (e.g., a srcRCC or a ccRCC), e.g., comprising a deletion of an MTAP gene, or of a portion thereof, and one or more mutations in an NF2 gene; or a treatment, a therapy, or one or more treatment options (e.g., an NF2-targeted therapy) for an individual having a cancer, such as a cancer of the disclosure (e.g., a srcRCC or a ccRCC, e.g., comprising a deletion of an MTAP gene, or of a portion thereof, and one or more mutations in an NF2 gene). In some embodiments, a report according to the present disclosure comprises information about one or more of: a deletion of an MTAP gene, or of a portion thereof, and one or more mutations in an NF2 gene; a cancer of the disclosure (e.g., a srcRCC or a ccRCC, e.g., comprising a deletion of an MTAP gene, or of a portion thereof, and one or more mutations in an NF2 gene); or a treatment, a therapy, or one or more treatment options (e.g., an NF2-targeted therapy) for an individual having a cancer, such as a cancer of the disclosure (e.g., a srcRCC or a ccRCC, e.g., comprising a deletion of an MTAP gene, or of a portion thereof, and one or more mutations in an NF2 gene). In some embodiments, the report further comprises information about one or more of: a deletion of a CDKN2A gene, or of a portion thereof; and/or a deletion of a CDKN2B gene, or of a portion thereof, e.g., a heterozygous or a homozygous deletion of a CDKN2A gene, or of a portion thereof; and/or a heterozygous or a homozygous deletion of a CDKN2B gene, or of a portion thereof. In some embodiments, the report comprises information about one or more mutations in one or more genes selected from CDKN2A, CDKN2B, VHL, PBRM1, SETD2, BAP1, TP53, PTEN, TERT, TSC1, PIK3CA, ATM, NF2, or MET.

In some embodiments, a report according to the present disclosure comprises information about the presence or absence of a deletion of an MTAP gene, or of a portion thereof, and one or more mutations in an NF2 gene in a sample obtained from an individual, such as an individual having a cancer, e.g., a cancer provided herein, e.g., a srcRCC or a ccRCC. In one embodiment, a report according to the present disclosure indicates that a deletion of an MTAP gene, or of a portion thereof, and one or more mutations in an NF2 gene are present in a sample obtained from the individual. In one embodiment, a report according to the present disclosure indicates that a deletion of an MTAP gene, or of a portion thereof, and one or more mutations in an NF2 gene are not present in a sample obtained from the individual. In one embodiment, a report according to the present disclosure indicates that a deletion of an MTAP gene, or of a portion thereof, and one or more mutations in an NF2 gene have been detected in a sample obtained from the individual. In one embodiment, a report according to the present disclosure indicates that a deletion of an MTAP gene, or of a portion thereof, and one or more mutations in an NF2 gene have not been detected in a sample obtained from the individual. In some embodiments, the report comprises an identifier for the individual from which the sample was obtained. In some embodiments, a report according to the present disclosure comprises information about the presence or absence of a deletion of a CDKN2A gene, or of a portion thereof, and/or a deletion of a CDKN2B gene, or of a portion thereof, in a sample obtained from an individual, such as an individual having a cancer, e.g., a cancer provided herein, e.g., a srcRCC or a ccRCC. In some embodiments, a report according to the present disclosure comprises information about the presence or absence of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual, such as an individual having a cancer, e.g., a cancer provided herein, e.g., a srcRCC or a ccRCC. In one embodiment, a report according to the present disclosure indicates that a deletion of a CDKN2A gene, or of a portion thereof, and/or a deletion of a CDKN2B gene, or of a portion thereof, are present in a sample obtained from the individual. In one embodiment, a report according to the present disclosure further indicates that one or more mutations in one or more genes selected from CDKN2A, CDKN2B, VHL, PBRM1, SETD2, BAP1, TP53, PTEN, TERT, TSC1, PIK3CA, ATM, NF2, or MET are present in a sample obtained from the individual. In one embodiment, a report according to the present disclosure further indicates that a deletion of a CDKN2A gene, or of a portion thereof, and/or a deletion of a CDKN2B gene, or of a portion thereof, are not present in a sample obtained from the individual. In one embodiment, a report according to the present disclosure further indicates that one or more mutations in one or more genes selected from CDKN2A, CDKN2B, VHL, PBRM1, SETD2, BAP1, TP53, PTEN, TERT, TSC1, PIK3CA, ATM, NF2, or MET are not present in a sample obtained from the individual. In one embodiment, a report according to the present disclosure indicates that a deletion of a CDKN2A gene, or of a portion thereof, and/or a deletion of a CDKN2B gene, or of a portion thereof, have been detected in a sample obtained from the individual. In one embodiment, a report according to the present disclosure indicates that one or more mutations in one or more genes selected from CDKN2A, CDKN2B, VHL, PBRM1, SETD2, BAP1, TP53, PTEN, TERT, TSC1, PIK3CA, ATM, NF2, or MET have been detected in a sample obtained from the individual. In one embodiment, a report according to the present disclosure indicates that a deletion of a CDKN2A gene, or of a portion thereof, and/or a deletion of a CDKN2B gene, or of a portion thereof, have not been detected in a sample obtained from the individual. In one embodiment, a report according to the present disclosure indicates that one or more mutations in one or more genes selected from CDKN2A, CDKN2B, VHL, PBRM1, SETD2, BAP1, TP53, PTEN, TERT, TSC1, PIK3CA, ATM, NF2, or MET have not been detected in a sample obtained from the individual. In some embodiments, the report comprises an identifier for the individual from which the sample was obtained.

In some embodiments, the report includes information on the role of a deletion of an MTAP gene, or of a portion thereof, and one or more mutations in an NF2 gene, or of the corresponding wild type counterparts, in disease, such as in cancer, e.g., a srcRCC or a ccRCC. Such information can include one or more of: information on prognosis of a cancer, such as a cancer provided herein, e.g., comprising a deletion of an MTAP gene, or of a portion thereof, and one or more mutations in an NF2 gene; information on resistance of a cancer, such as a cancer provided herein, e.g., a srcRCC or a ccRCC, such as a srcRCC or a ccRCC comprising a deletion of an MTAP gene, or of a portion thereof, and one or more mutations in an NF2 gene, to one or more treatments; information on potential or suggested therapeutic options (e.g., such as an anti-cancer therapy provided herein, or a treatment selected or identified according to the methods provided herein, e.g., an NF2-targeted therapy); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein, e.g., an NF2-targeted therapy) to an individual having a cancer, such as a cancer provided herein, e.g., a srcRCC or a ccRCC, e.g., comprising a deletion of an MTAP gene, or of a portion thereof, and one or more mutations in an NF2 gene, and identified in the report. In some embodiments, the report includes information or a recommendation on the administration of a treatment (e.g., an anti-cancer therapy provided herein, or a treatment selected or identified according to the methods provided herein, such as an NF2-targeted therapy). In some embodiments, the report further includes information on the role of a deletion of a CDKN2A gene, or of a portion thereof, and/or a deletion of a CDKN2B gene, or of a portion thereof, or of the corresponding wild type counterparts, in disease, such as in cancer, e.g., a srcRCC or a ccRCC. In some embodiments, the report further includes information on the role of one or more mutations in one or more genes selected from CDKN2A, CDKN2B, VHL, PBRM1, SETD2, BAP1, TP53, PTEN, TERT, TSC1, PIK3CA, ATM, NF2, or MET, or of the corresponding wild type counterparts, in disease, such as in cancer, e.g., a srcRCC or a ccRCC. Such information can include one or more of: information on prognosis of a cancer, such as a cancer provided herein, e.g., comprising a deletion of a CDKN2A gene, or of a portion thereof, and/or a deletion of a CDKN2B gene, or of a portion thereof; information on resistance of a cancer, such as a cancer provided herein, e.g., a srcRCC or a ccRCC, such as a srcRCC or a ccRCC comprising a deletion of a CDKN2A gene, or of a portion thereof, and/or a deletion of a CDKN2B gene, or of a portion thereof, to one or more treatments; information on potential or suggested therapeutic options (e.g., such as an anti-cancer therapy provided herein, or a treatment selected or identified according to the methods provided herein, e.g., an NF2-targeted therapy); or information on therapeutic options that should be avoided. Such information can include one or more of: information on prognosis of a cancer, such as a cancer provided herein, e.g., comprising one or more mutations in one or more genes selected from CDKN2A, CDKN2B, VHL, PBRM1, SETD2, BAP1, TP53, PTEN, TERT, TSC1, PIK3CA, ATM, NF2, or MET; information on resistance of a cancer, such as a cancer provided herein, e.g., a srcRCC or a ccRCC, such as a srcRCC or a ccRCC comprising one or more mutations in one or more genes selected from CDKN2A, CDKN2B, VHL, PBRM1, SETD2, BAP1, TP53, PTEN, TERT, TSC1, PIK3CA, ATM, NF2, or MET, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., a srcRCC or a ccRCC, e.g., comprising a deletion of a CDKN2A gene, or of a portion thereof, and/or a deletion of a CDKN2B gene, or of a portion thereof, and identified in the report. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., a srcRCC or a ccRCC, e.g., comprising one or more mutations in one or more genes selected from CDKN2A, CDKN2B, VHL, PBRM1, SETD2, BAP1, TP53, PTEN, TERT, TSC1, PIK3CA, ATM, NF2, or MET, and identified in the report. In some embodiments, the report includes information or a recommendation on the administration of a treatment (e.g., an anti-cancer therapy provided herein, or a treatment selected or identified according to the methods provided herein, such as an NF2-targeted therapy). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein (e.g., a srcRCC or a ccRCC); detecting a deletion of an MTAP gene, or of a portion thereof, and one or more mutations in an NF2 gene in the sample, or acquiring knowledge of the presence of a deletion of an MTAP gene, or of a portion thereof, and one or more mutations in an NF2 gene in the sample; and generating a report. In some embodiments, the method comprises detecting a deletion of a CDKN2A gene, or of a portion thereof, and/or a deletion of a CDKN2B gene, or of a portion thereof, in the sample, or acquiring knowledge of the presence of a deletion of a CDKN2A gene, or of a portion thereof, and/or a deletion of a CDKN2B gene, or of a portion thereof, in the sample. In some embodiments, the method comprises detecting one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, or acquiring knowledge of the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, a report generated according to the methods provided herein comprises one or more of: information about the presence or absence of a deletion of an MTAP gene, or of a portion thereof, and one or more mutations in an NF2 gene in the sample; an identifier for the individual from which the sample was obtained; information on the role of a deletion of an MTAP gene, or of a portion thereof, and one or more mutations in an NF2 gene, or the corresponding wild type counterparts, in disease (e.g., such as in cancer, e.g., a srcRCC or a ccRCC); information on prognosis, resistance, or potential or suggested therapeutic options (such as an anti-cancer therapy provided herein, or a treatment selected or identified according to the methods provided herein, such as an NF2-targeted therapy); information on the likely effectiveness, acceptability, or the advisability of applying a therapeutic option (such as an anti-cancer therapy provided herein, or a treatment selected or identified according to the methods provided herein, e.g., an NF2-targeted therapy) to the individual; a recommendation or information on the administration of a treatment (such as an anti-cancer therapy provided herein, or a treatment selected or identified according to the methods provided herein, e.g., an NF2-targeted therapy); or a recommendation or information on the dosage or treatment regimen of a treatment (such as an anti-cancer therapy provided herein, or a treatment selected or identified according to the methods provided herein, e.g., an NF2-targeted therapy), e.g., in combination with other treatments (e.g., a second therapeutic agent). In some embodiments, the report comprises information about the presence or absence of a deletion of a CDKN2A gene, or of a portion thereof, and/or a deletion of a CDKN2B gene, or of a portion thereof, in the sample. In some embodiments, the report comprises information about the presence or absence of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, the report generated is a personalized cancer report.

A report according to the present disclosure may be in an electronic, web-based, or paper form. The report may be provided to an individual or a patient, e.g., an individual or a patient with a cancer, such as a cancer provided herein (e.g., a srcRCC or a ccRCC, such as a srcRCC or a ccRCC comprising a deletion of an MTAP gene, or of a portion thereof, and one or more mutations in an NF2 gene, and optionally a deletion of a CDKN2A gene, or of a portion thereof, and/or a deletion of a CDKN2B gene, or of a portion thereof), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a deletion of an MTAP gene, or of a portion thereof, and one or more mutations in an NF2 gene, and optionally a deletion of a CDKN2A gene, or of a portion thereof, and/or a deletion of a CDKN2B gene, or of a portion thereof, in a sample obtained from an individual (e.g., an individual having a cancer, e.g., a srcRCC or a ccRCC). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a deletion of an MTAP gene, or of a portion thereof, and one or more mutations in an NF2 gene, and optionally a deletion of a CDKN2A gene, or of a portion thereof, and/or a deletion of a CDKN2B gene, or of a portion thereof, in a sample obtained from an individual (e.g., an individual having a cancer). The report may be provided to an individual or a patient (e.g., an individual or a patient with a cancer, such as a cancer provided herein, e.g., a srcRCC or a ccRCC, such as a srcRCC or a ccRCC comprising a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual (e.g., an individual having a cancer, e.g., a srcRCC or a ccRCC). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual (e.g., an individual having a cancer).

In some embodiments of any of the reports or methods provided herein, the potential or suggested therapeutic options include a PRMT5-targeted therapy and an additional anti-cancer therapy, e.g., an anti-cancer therapy provided herein or an NF2-targeted therapy. In some embodiments, the report includes information on the likely effectiveness, acceptability, and/or advisability of applying a PRMT5-targeted therapy and an additional anti-cancer therapy, e.g., an anti-cancer therapy provided herein or an NF2-targeted therapy, to an individual having a cancer, such as a cancer provided herein (e.g., a srcRCC or a ccRCC), e.g., comprising a deletion of an MTAP gene, or of a portion thereof, and one or more mutations in an NF2 gene, and identified in the report. In some embodiments, the report includes information or a recommendation on the administration of a PRMT5-targeted therapy and an additional anti-cancer therapy, e.g., an NF2-targeted therapy. In some embodiments, the report includes information on the likely effectiveness, acceptability, and/or advisability of applying a PRMT5-targeted therapy and an additional anti-cancer therapy, e.g., an anti-cancer therapy provided herein, to an individual having a cancer, such as a cancer provided herein (e.g., a srcRCC or a ccRCC), e.g., comprising a deletion of an MTAP gene, or of a portion thereof, and one or more mutations in one or more genes selected from CDKN2A, CDKN2B, VHL, PBRM1, SETD2, BAP1, TP53, PTEN, TERT, TSC1, PIK3CA, ATM, NF2, or MET, and identified in the report. In some embodiments, the report includes information or a recommendation on the administration of a PRMT5-targeted therapy and an additional anti-cancer therapy, e.g., an anti-cancer therapy provided herein.

### MTAP-Intact Renal Cell Carcinoma

In some aspects, provided herein are methods of treating or delaying progression of a clear cell renal cell carcinoma (ccRCC) in an individual, wherein the ccRCC does not comprise a deletion of an methylthioadenosine phosphorylase (MTAP) gene, or of a portion thereof (MTAP-intact ccRCC).

In other aspects, methods are provided for identifying one or more treatment options for an individual having an MTAP-intact ccRCC. In other aspects, methods are provided for selecting treatment for an individual having an MTAP-intact ccRCC. In some aspects, methods are provided for predicting survival of an individual having an MTAP-intact ccRCC treated with an anti-cancer therapy. In other aspects, provided herein are methods of identifying an individual having an MTAP-intact ccRCC, who may benefit from a treatment comprising an anti-cancer therapy. In other aspects, provided herein are methods of selecting a therapy for an individual having an MTAP-intact ccRCC. In other aspects, provided herein are methods of assessing, screening, or diagnosing an individual having an MTAP-intact ccRCC.

In some embodiments, the methods comprise acquiring knowledge of or detecting in a sample from an individual having an MTAP-intact ccRCC one or more mutations in a VHL gene. In some embodiments, the methods comprise acquiring knowledge of or detecting in a sample from an individual having an MTAP-intact ccRCC one or more mutations in a PBRM1 gene. In some embodiments, the methods comprise acquiring knowledge of or detecting in a sample from an individual having an MTAP-intact ccRCC, one or more mutations in one or more genes, wherein the one or more genes comprise CDKN2A, CDKN2B, VHL, PBRM1, SETD2, BAP1, TP53, PTEN, TERT, TSC1, PIK3CA, ATM, NF2, or MET.

Methods of Selecting or Identifying a Treatment

In some aspects, provided herein are methods of identifying an individual having cancer, e.g., a ccRCC, who may benefit from an anti-cancer therapy, e.g., an anti-cancer therapy provided herein. In some embodiments, the methods comprise detecting one or more mutations in a VHL gene or a PBRM1 gene in a sample obtained from the individual. In some embodiments, the methods comprise acquiring knowledge of the presence of one or more mutations in a VHL gene or a PBRM1 gene in a sample obtained from the individual. In some embodiments, the presence of one or more mutations in a VHL gene or a PBRM1 gene in the sample identifies the individual as one who may benefit from a treatment comprising an anti-cancer therapy, e.g., an anti-cancer therapy provided herein, such as a VHL-targeted therapy or a PBRM1-targeted therapy. In some embodiments, detection of the one or more mutations in a VHL gene or a PBRM1 gene in the sample identifies the individual as one who may benefit from a treatment comprising an anti-cancer therapy, e.g., an anti-cancer therapy provided herein, such as a VHL-targeted therapy or a PBRM1-targeted therapy. In some embodiments, responsive to knowledge of one or more mutations in a VHL gene or a PBRM1 gene in the sample, the individual is identified as one who may benefit from a treatment comprising an anti-cancer therapy, e.g., an anti-cancer therapy provided herein, such as a VHL-targeted therapy or a PBRM1-targeted therapy. In some embodiments, the presence of one or more mutations in a VHL gene in the sample identifies the individual as one who may benefit from a treatment comprising an anti-cancer therapy, e.g., an anti-cancer therapy provided herein, such as a VHL-targeted therapy. In some embodiments, detection of one or more mutations in a VHL gene in the sample identifies the individual as one who may benefit from a treatment comprising an anti-cancer therapy, e.g., an anti-cancer therapy provided herein, such as a VHL-targeted therapy. In some embodiments, responsive to knowledge of one or more mutations in a VHL gene in the sample, the individual is identified as one who may benefit from a treatment comprising an anti-cancer therapy, e.g., an anti-cancer therapy provided herein, such as a VHL-targeted therapy. In some embodiments, the presence of one or more mutations in a PBRM1 gene in the sample identifies the individual as one who may benefit from a treatment comprising an anti-cancer therapy, e.g., an anti-cancer therapy provided herein, such as a PBRM1-targeted therapy. In some embodiments, detection of one or more mutations in a PBRM1 gene in the sample identifies the individual as one who may benefit from a treatment comprising an anti-cancer therapy, e.g., an anti-cancer therapy provided herein, such as a PBRM1-targeted therapy. In some embodiments, responsive to knowledge of one or more mutations in a PBRM1 gene in the sample, the individual is identified as one who may benefit from a treatment comprising an anti-cancer therapy, e.g., an anti-cancer therapy provided herein, such as a PBRM1-targeted therapy. In some embodiments, one or more mutations in a VHL gene or a PBRM1 gene are detected using any suitable method known in the art or described herein. In some embodiments, the sample is a sample described herein. In some embodiments, the sample comprises cells from the cancer or is obtained from cells from the cancer, e.g., a ccRCC.

Also provided herein are methods of identifying or selecting a treatment, a therapy, or one or more treatment options for an individual having a cancer, such as a cancer described herein, e.g., a ccRCC. In some embodiments, the cancer comprises one or more mutations in a VHL gene or a PBRM1 gene. In some embodiments, the methods comprise detecting one or more mutations in a VHL gene or a PBRM1 gene in a sample obtained from an individual having a cancer, such as a cancer described herein, e.g., a ccRCC. In some embodiments, the one or more mutations in a VHL gene or a PBRM1 gene are detected using any suitable method known in the art or described herein. In some embodiments, detection of one or more mutations in a VHL gene or a PBRM1 gene in a sample obtained from an individual having a cancer, e.g., a cancer described herein, e.g., a ccRCC, identifies the individual as one who may benefit from an anti-cancer therapy, e.g., an anti-cancer therapy provided herein, such as a VHL-targeted therapy or a PBRM1-targeted therapy. In some embodiments, the presence of one or more mutations in a VHL gene or a PBRM1 gene in a sample obtained from an individual having a cancer, e.g., a cancer provided herein, e.g., a ccRCC, identifies the individual as one who may benefit from an anti-cancer therapy, such as an anti-cancer therapy provided herein, e.g., a VHL-targeted therapy or a PBRM1-targeted therapy. In some embodiments, responsive to detection of one or more mutations in a VHL gene or a PBRM1 gene in a sample obtained from an individual having a cancer, e.g., a cancer provided herein, e.g., a ccRCC, the individual is classified as a candidate to receive an anti-cancer therapy, such as an anti-cancer therapy provided herein, e.g., a VHL-targeted therapy or a PBRM1-targeted therapy. In some embodiments, responsive to detection of one or more mutations in a VHL gene or a PBRM1 gene in a sample obtained from an individual having a cancer, e.g., a cancer provided herein, e.g., a ccRCC, the individual is classified or identified as likely to respond to an anti-cancer therapy, such as an anti-cancer therapy provided herein, e.g., a VHL-targeted therapy or a PBRM1-targeted therapy. In some embodiments, detection of one or more mutations in a VHL gene in a sample obtained from an individual having a cancer, e.g., a cancer described herein, e.g., a ccRCC, identifies the individual as one who may benefit from an anti-cancer therapy, e.g., an anti-cancer therapy provided herein, such as a VHL-targeted therapy. In some embodiments, the presence of one or more mutations in a VHL gene in a sample obtained from an individual having a cancer, e.g., a cancer provided herein, e.g., a ccRCC, identifies the individual as one who may benefit from an anti-cancer therapy, such as an anti-cancer therapy provided herein, e.g., a VHL-targeted therapy. In some embodiments, responsive to detection of one or more mutations a VHL gene in a sample obtained from an individual having a cancer, e.g., a cancer provided herein, e.g., a ccRCC, the individual is classified as a candidate to receive an anti-cancer therapy, such as an anti-cancer therapy provided herein, e.g., a VHL-targeted therapy. In some embodiments, responsive to detection of one or more mutations in a VHL gene in a sample obtained from an individual having a cancer, e.g., a cancer provided herein, e.g., a ccRCC, the individual is classified or identified as likely to respond to an anti-cancer therapy, such as an anti-cancer therapy provided herein, e.g., a VHL-targeted therapy. In some embodiments, detection of one or more mutations in a PBRM1 gene in a sample obtained from an individual having a cancer, e.g., a cancer described herein, e.g., a ccRCC, identifies the individual as one who may benefit from an anti-cancer therapy, e.g., an anti-cancer therapy provided herein, such as a PBRM1-targeted therapy. In some embodiments, the presence of one or more mutations in a PBRM1 gene in a sample obtained from an individual having a cancer, e.g., a cancer provided herein, e.g., a ccRCC, identifies the individual as one who may benefit from an anti-cancer therapy, such as an anti-cancer therapy provided herein, e.g., a PBRM1-targeted therapy. In some embodiments, responsive to detection of one or more mutations in a PBRM1 gene in a sample obtained from an individual having a cancer, e.g., a cancer provided herein, e.g., a ccRCC, the individual is classified as a candidate to receive an anti-cancer therapy, such as an anti-cancer therapy provided herein, e.g., a PBRM1-targeted therapy. In some embodiments, responsive to detection of one or more mutations in a PBRM1 gene in a sample obtained from an individual having a cancer, e.g., a cancer provided herein, e.g., a ccRCC, the individual is classified or identified as likely to respond to an anti-cancer therapy, such as an anti-cancer therapy provided herein, e.g., a PBRM1-targeted therapy. In some embodiments, the sample is a sample described herein. In some embodiments, the sample comprises cells from the cancer or is obtained from cells from the cancer, e.g., a ccRCC. In some embodiments, the methods further comprise generating a report, e.g., as described herein. In some embodiments, the report comprises a treatment, a therapy, or one or more treatment options identified or selected for the individual, e.g., based at least in part on detection of one or more mutations in a VHL gene or a PBRM1 gene in the sample.

In some aspects, provided herein are methods of identifying an individual having cancer, e.g., a ccRCC, who may benefit from an anti-cancer therapy, e.g., an anti-cancer therapy provided herein. In some embodiments, the methods comprise detecting one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, the methods comprise acquiring knowledge of the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, the presence of the one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the individual as one who may benefit from a treatment comprising an anti-cancer therapy, e.g., an anti-cancer therapy provided herein. In some embodiments, detection of the one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the individual as one who may benefit from a treatment comprising an anti-cancer therapy, e.g., an anti-cancer therapy provided herein. In some embodiments, responsive to knowledge of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is identified as one who may benefit from a treatment comprising an anti-cancer therapy, e.g., an anti-cancer therapy provided herein. In some embodiments, the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the individual as one who may benefit from a treatment comprising an anti-cancer therapy, e.g., an anti-cancer therapy provided herein. In some embodiments, detection of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the individual as one who may benefit from a treatment comprising an anti-cancer therapy, e.g., an anti-cancer therapy provided herein. In some embodiments, responsive to knowledge of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is identified as one who may benefit from a treatment comprising an anti-cancer therapy, e.g., an anti-cancer therapy provided herein. In some embodiments, the one or more mutations in one or more genes selected from CDKN2A, CDKN2B, VHL, PBRM1, SETD2, BAP1, TP53, PTEN, TERT, TSC1, PIK3CA, ATM, NF2, or MET are detected using any suitable method known in the art or described herein. In some embodiments, the sample is a sample described herein. In some embodiments, the sample comprises cells from the cancer or is obtained from cells from the cancer, e.g., a ccRCC.

Also provided herein are methods of identifying or selecting a treatment, a therapy, or one or more treatment options for an individual having a cancer, such as a cancer described herein. In some embodiments, the cancer comprises one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the methods comprise detecting one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, such as a cancer described herein, e.g., a ccRCC. In some embodiments, the one or more mutations in one or more genes selected from CDKN2A, CDKN2B, VHL, PBRM1, SETD2, BAP1, TP53, PTEN, TERT, TSC1, PIK3CA, ATM, NF2, or MET are detected using any suitable method known in the art or described herein. In some embodiments, detection of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer described herein, e.g., a ccRCC, identifies the individual as one who may benefit from an anti-cancer therapy, e.g., an anti-cancer therapy provided herein. In some embodiments, the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a ccRCC, identifies the individual as one who may benefit from an anti-cancer therapy, such as an anti-cancer therapy provided herein. In some embodiments, responsive to detection of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a ccRCC, the individual is classified as a candidate to receive an anti-cancer therapy, such as an anti-cancer therapy provided herein. In some embodiments, responsive to detection of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a ccRCC, the individual is classified or identified as likely to respond to an anti-cancer therapy, such as an anti-cancer therapy provided herein. In some embodiments, detection of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer described herein, e.g., a ccRCC, identifies the individual as one who may benefit from an anti-cancer therapy, e.g., an anti-cancer therapy provided herein. In some embodiments, the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a ccRCC, identifies the individual as one who may benefit from an anti-cancer therapy, such as an anti-cancer therapy provided herein. In some embodiments, responsive to detection of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a ccRCC, the individual is classified as a candidate to receive an anti-cancer therapy, such as an anti-cancer therapy provided herein. In some embodiments, responsive to detection of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual having a cancer, e.g., a cancer provided herein, e.g., a ccRCC, the individual is classified or identified as likely to respond to an anti-cancer therapy, such as an anti-cancer therapy provided herein. In some embodiments, the sample is a sample described herein. In some embodiments, the sample comprises cells from the cancer or is obtained from cells from the cancer, e.g., a ccRCC. In some embodiments, the methods further comprise generating a report, e.g., as described herein. In some embodiments, the report comprises a treatment, a therapy, or one or more treatment options identified or selected for the individual, e.g., based at least in part on detection of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample.

In some embodiments of any of the methods provided herein, the methods further comprise acquiring knowledge of or detecting in a sample from an individual the absence of a deletion of an MTAP gene, or of a portion thereof. In some embodiments of any of the methods provided herein, the individual does not have a deletion of an MTAP gene, or of a portion thereof.

Methods of Treatment

Also provided herein are methods of treating or delaying progression of a cancer in an individual, such as a cancer provided herein, e.g., a ccRCC. In some embodiments, the individual has a cancer comprising one or more mutations in a VHL gene or a PBRM1 gene. In some embodiments, the methods of treating or delaying progression of a cancer of the disclosure, e.g., a ccRCC, comprising one or more mutations in a VHL gene or a PBRM1 gene, comprise administering to an individual a therapeutically effective amount of an anti-cancer therapy, such as an anti-cancer therapy provided herein, e.g., a VHL-targeted therapy or a PBRM1-targeted therapy. In some embodiments, the methods of treating or delaying progression of a cancer of the disclosure, e.g., a ccRCC, comprising one or more mutations in a VHL gene or a PBRM1 gene, comprise administering to an individual an effective amount of an anti-cancer therapy, such as an anti-cancer therapy provided herein (e.g., a VHL-targeted therapy or a PBRM1-targeted therapy), responsive to knowledge of the presence of the one or more mutations a VHL gene or a PBRM1 gene in a sample obtained from the individual. In some embodiments, the sample is a sample described herein. In some embodiments, the sample comprises cells from the cancer or is obtained from cells from the cancer, e.g. a ccRCC. The methods of treatment disclosed herein may include any of the anti-cancer therapies and/or therapeutic agents described herein, e.g., infra.

In some embodiments, the individual has a cancer comprising one or more mutations in a VHL gene. In some embodiments, the methods of treating or delaying progression of a cancer of the disclosure, e.g., a ccRCC comprising one or more mutations in a VHL gene, comprise administering to an individual a therapeutically effective amount of an anti-cancer therapy, such as an anti-cancer therapy provided herein, e.g., a VHL-targeted therapy. In some embodiments, the methods of treating or delaying progression of a cancer of the disclosure, e.g., a ccRCC comprising one or more mutations in a VHL gene, comprise administering to the individual an effective amount of an anti-cancer therapy, such as an anti-cancer therapy provided herein (e.g., a VHL-targeted therapy), responsive to knowledge of the presence of the one or more mutations in a VHL gene in a sample obtained from the individual. In some embodiments, the VHL-targeted therapy comprises one or more of a poly(ADP-ribose) polymerase (PARP) inhibitor, a glutaminase 1 (GLS-1) inhibitor, a VEGF inhibitor, a HIF-2alpha inhibitor, an HDAC inhibitor, a CDK4/6 inhibitor, a Tank binding kinase 1 (TBK1) inhibitor, an EZH1 and/or EZH2 inhibitor, a Rho-Associated Kinase 1 (ROCK1) inhibitor, a glucose transporter 1 (GLUT1) inhibitor, an autophagy modulator, or an immune checkpoint inhibitor.

In some embodiments, the individual has a cancer comprising one or more mutations in a PBRM1 gene. In some embodiments, the methods of treating or delaying progression of a cancer of the disclosure, e.g., a ccRCC comprising one or more mutations in a PBRM1 gene, comprise administering to an individual a therapeutically effective amount of an anti-cancer therapy, such as an anti-cancer therapy provided herein, e.g., a PBRM1-targeted therapy. In some embodiments, the methods of treating or delaying progression of a cancer of the disclosure, e.g., a ccRCC comprising one or more mutations in a PBRM1 gene, comprise administering to the individual an effective amount of an anti-cancer therapy, such as an anti-cancer therapy provided herein (e.g., a PBRM1-targeted therapy), responsive to knowledge of the presence of the one or more mutations in a PBRM1 gene in a sample obtained from the individual. In some embodiments, the PBRM1-targeted therapy comprises one or more of an EZH2 inhibitor, a VEGF inhibitor, or an mTOR inhibitor.

Also provided herein are methods of treating or delaying progression of a cancer in an individual, such as a cancer provided herein, e.g., a ccRCC. In some embodiments, the individual has a cancer comprising one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the methods of treating or delaying progression of a cancer of the disclosure, e.g., a ccRCC comprising one or more mutations in one or more genes selected from CDKN2A, CDKN2B, VHL, PBRM1, SETD2, BAP1, TP53, PTEN, TERT, TSC1, PIK3CA, ATM, NF2, or MET, comprise administering to an individual a therapeutically effective amount of an anti-cancer therapy, such as an anti-cancer therapy provided herein. In some embodiments, the methods of treating or delaying progression of a cancer of the disclosure, e.g., a ccRCC comprising one or more mutations in one or more genes selected from CDKN2A, CDKN2B, VHL, PBRM1, SETD2, BAP1, TP53, PTEN, TERT, TSC1, PIK3CA, ATM, NF2, or MET, comprise administering to an individual an effective amount of an anti-cancer therapy, such as an anti-cancer therapy provided herein, responsive to knowledge of the presence of the one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, the sample is a sample described herein. In some embodiments, the sample comprises cells from the cancer or is obtained from cells from the cancer, e.g. a ccRCC. The methods of treatment disclosed herein may include any of the anti-cancer therapies and/or therapeutic agents described herein, e.g., infra.

In some embodiments, the individual has a cancer comprising one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the methods of treating or delaying progression of a cancer of the disclosure, e.g., a ccRCC comprising one or more mutations in one or more genes selected from CDKN2A, CDKN2B, VHL, PBRM1, SETD2, BAP1, TP53, PTEN, TERT, TSC1, PIK3CA, ATM, NF2, or MET, comprise administering to an individual a therapeutically effective amount of an anti-cancer therapy, such as an anti-cancer therapy provided herein. In some embodiments, the methods of treating or delaying progression of a cancer of the disclosure, e.g., a ccRCC comprising one or more mutations in one or more genes selected from CDKN2A, CDKN2B, VHL, PBRM1, SETD2, BAP1, TP53, PTEN, TERT, TSC1, PIK3CA, ATM, NF2, or MET, comprise administering to the individual an effective amount of an anti-cancer therapy, such as an anti-cancer therapy provided herein, responsive to knowledge of the presence of the one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, the anti-cancer therapy is administered in combination with a second anti-cancer therapy or agent, such as any of the anti-cancer agents or therapies described herein.

In some embodiments of any of the methods provided herein, the methods further comprise acquiring knowledge of or detecting in a sample from an individual the absence of a deletion of an MTAP gene, or of a portion thereof. In some embodiments of any of the methods provided herein, the individual does not have a deletion of an MTAP gene, or of a portion thereof.

Methods of Diagnosing, Assessing, Screening, Monitoring or Predicting

In some aspects, provided herein are methods of diagnosing or assessing one or more mutations in a VHL gene or a PBRM1 gene in a sample from an individual having a cancer, such as a ccRCC. In some embodiments, the methods comprise acquiring knowledge of the presence of one or more mutations in a VHL gene or a PBRM1 gene in a sample obtained from the individual. In some embodiments, the methods comprise detecting one or more mutations in a VHL gene or a PBRM1 gene in a sample obtained from the individual. In some embodiments, one or more mutations in a VHL gene or a PBRM1 gene are detected in a sample obtained from the individual using any method known in the art, such as one or more of the methods of detection of nucleic acid molecules or polypeptides described herein. In some embodiments, the methods further comprise providing a diagnosis or an assessment of the one or more mutations in a VHL gene or a PBRM1 gene. In some embodiments, the diagnosis or assessment identifies the presence or absence of one or more mutations in a VHL gene or a PBRM1 gene in the sample. In some embodiments, the diagnosis or assessment identifies the cancer, such as a ccRCC provided herein, as likely to respond to an anti-cancer therapy, e.g., an anti-cancer therapy provided herein, such as a VHL-targeted therapy or a PBRM1-targeted therapy. In some embodiments, the presence of one or more mutations in a VHL gene in the sample identifies the cancer as likely to respond to an anti-cancer therapy, e.g., an anti-cancer therapy provided herein, such as a VHL-targeted therapy. In some embodiments, the diagnosis or assessment identifies the presence or absence of one or more mutations in a VHL gene in the sample. In some embodiments, the diagnosis or assessment identifies the cancer, such as a ccRCC provided herein, as likely to respond to an anti-cancer therapy, e.g., an anti-cancer therapy provided herein, such as a VHL-targeted therapy. In some embodiments, the presence of one or more mutations in a VHL gene in the sample identifies the cancer as likely to respond to an anti-cancer therapy, e.g., an anti-cancer therapy provided herein, such as a VHL-targeted therapy. In some embodiments, the presence of one or more mutations in a PBRM1 gene in the sample identifies the cancer as likely to respond to an anti-cancer therapy, e.g., an anti-cancer therapy provided herein, such as a PBRM1-targeted therapy. In some embodiments, the diagnosis or assessment identifies the presence or absence of one or more mutations in a PBRM1 gene in the sample. In some embodiments, the diagnosis or assessment identifies the cancer, such as a ccRCC provided herein, as likely to respond to an anti-cancer therapy, e.g., an anti-cancer therapy provided herein, such as a PBRM1-targeted therapy. In some embodiments, the presence of one or more mutations in a PBRM1 gene in the sample identifies the cancer as likely to respond to an anti-cancer therapy, e.g., an anti-cancer therapy provided herein, such as a PBRM1-targeted therapy. In some embodiments, the sample is a sample described herein. In some embodiments, the sample comprises cells from the cancer or is obtained from cells from the cancer, e.g., from a ccRCC. In some embodiments, the individual has a cancer, is suspected of having a cancer, is being tested for a cancer, is being treated for a cancer, or is being tested for a susceptibility to a cancer, e.g., a ccRCC.

In some aspects, provided herein are methods of diagnosing or assessing a cancer in an individual, e.g., a ccRCC. In some embodiments, the methods of diagnosing or assessing cancer comprise detecting one or more mutations in a VHL gene or a PBRM1 gene in a sample obtained from the individual, e.g., a sample comprising cells from the cancer, e.g., a ccRCC. In some embodiments, the methods comprise detecting one or more mutations in a VHL gene or a PBRM1 gene in a sample obtained from the individual using any method known in the art, such as one or more of the methods of detection of nucleic acid molecules or polypeptides described herein. In some embodiments, detection of one or more mutations in a VHL gene or a PBRM1 gene in a sample obtained from the individual identifies the cancer, e.g., the ccRCC, as likely to respond to an anti-cancer therapy, e.g., an anti-cancer therapy provided herein, such as a VHL-targeted therapy or a PBRM1-targeted therapy. In some embodiments, the presence of one or more mutations in a VHL gene or a PBRM1 gene in a sample obtained from the individual identifies the cancer, e.g., the ccRCC, as likely to respond to an anti-cancer therapy, e.g., an anti-cancer therapy provided herein, such as a VHL-targeted therapy or a PBRM1-targeted therapy. In some embodiments, the methods further comprise providing a diagnosis or an assessment of the cancer, e.g., the ccRCC, or of one or more mutations in a VHL gene or a PBRM1 gene. In some embodiments, the diagnosis or assessment identifies the cancer as likely to respond to an anti-cancer therapy, e.g., an anti-cancer therapy provided herein, such as a VHL-targeted therapy or a PBRM1-targeted therapy. In some embodiments, detection of one or more mutations in a VHL gene in a sample obtained from the individual identifies the cancer, e.g., the ccRCC, as likely to respond to an anti-cancer therapy, e.g., an anti-cancer therapy provided herein, such as a VHL-targeted therapy. In some embodiments, the presence of one or more mutations in a VHL gene in a sample obtained from the individual identifies the cancer, e.g., the ccRCC, as likely to respond to an anti-cancer therapy, e.g., an anti-cancer therapy provided herein, such as a VHL-targeted therapy. In some embodiments, the methods further comprise providing a diagnosis or an assessment of the cancer, e.g., the ccRCC, or of the one or more mutations in a VHL gene. In some embodiments, the diagnosis or assessment identifies the cancer as likely to respond to an anti-cancer therapy, e.g., an anti-cancer therapy provided herein, such as a VHL-targeted therapy. In some embodiments, the diagnosis or assessment identifies the presence or absence of the one or more mutations in a VHL gene in the sample. In some embodiments, detection of one or more mutations in a PBRM1 gene in a sample obtained from the individual identifies the cancer, e.g., the ccRCC, as likely to respond to an anti-cancer therapy, e.g., an anti-cancer therapy provided herein, such as a PBRM1-targeted therapy. In some embodiments, the presence of one or more mutations in a PBRM1 gene in a sample obtained from the individual identifies the cancer, e.g., the ccRCC, as likely to respond to an anti-cancer therapy, e.g., an anti-cancer therapy provided herein, such as a PBRM1-targeted therapy. In some embodiments, the methods further comprise providing a diagnosis or an assessment of the cancer, e.g., the ccRCC, or of the one or more mutations in a PBRM11 gene. In some embodiments, the diagnosis or assessment identifies the cancer as likely to respond to an anti-cancer therapy, e.g., an anti-cancer therapy provided herein, such as a PBRM1-targeted therapy. In some embodiments, the diagnosis or assessment identifies the presence or absence of the one or more mutations in a PBRM1 gene in the sample.

In some aspects, provided herein are methods of predicting survival of an individual having a cancer, e.g., a cancer provided herein, e.g., a ccRCC. In some embodiments, the individual is being treated with an anti-cancer therapy, such as an anti-cancer therapy described herein. In some embodiments, the methods comprise acquiring knowledge of one or more mutations in a VHL gene or a PBRM1 gene in a sample from the individual. In some embodiments, the methods comprise detecting one or more mutations in a VHL gene or a PBRM1 gene in a sample from the individual. In some embodiments, responsive to acquiring knowledge of one or more mutations in a VHL gene or a PBRM11 gene in the sample, the individual is predicted to have longer survival after treatment with an anti-cancer therapy, e.g., an anti-cancer therapy provided herein (e.g., a VHL-targeted therapy or a PBRM1-targeted therapy), for example, as compared to an individual whose cancer does not exhibit the one or more mutations in a VHL gene or a PBRM1 gene. In some embodiments, responsive to detecting one or more mutations in a VHL gene or a PBRM1 gene in the sample, the individual is predicted to have longer survival after treatment with an anti-cancer therapy, e.g., an anti-cancer therapy provided herein (e.g., a VHL-targeted therapy or a PBRM1-targeted therapy), for example, as compared to an individual whose cancer does not exhibit the one or more mutations in a VHL gene or a PBRM1 gene. In some embodiments, responsive to acquiring knowledge of one or more mutations in a VHL gene in the sample, the individual is predicted to have longer survival after treatment with an anti-cancer therapy, e.g., an anti-cancer therapy provided herein (e.g., a VHL-targeted therapy), for example, as compared to an individual whose cancer does not exhibit the one or more mutations in a VHL gene. In some embodiments, responsive to detecting one or more mutations in a VHL gene in the sample, the individual is predicted to have longer survival after treatment with an anti-cancer therapy, e.g., an anti-cancer therapy provided herein (e.g., a VHL-targeted therapy), for example, as compared to an individual whose cancer does not exhibit the one or more mutations in a VHL gene. In some embodiments, responsive to acquiring knowledge of one or more mutations in a PBRM1 gene in the sample, the individual is predicted to have longer survival after treatment with an anti-cancer therapy, e.g., an anti-cancer therapy provided herein (e.g., a PBRM1-targeted therapy), for example, as compared to an individual whose cancer does not exhibit the one or more mutations in a PBRM1 gene. In some embodiments, responsive to detecting one or more mutations in a PBRM1 gene in the sample, the individual is predicted to have longer survival after treatment with an anti-cancer therapy, e.g., an anti-cancer therapy provided herein (e.g., a PBRM1-targeted therapy), for example, as compared to an individual whose cancer does not exhibit the one or more mutations in a PBRM1 gene. In some embodiments, the methods further comprise providing a diagnosis or an assessment. In some embodiments, the diagnosis or assessment identifies the presence or absence of the one or more mutations in a VHL gene or a PBRM1 gene in the sample. In some embodiments, the diagnosis or assessment identifies the individual as being predicted to have longer survival after treatment with an anti-cancer therapy, e.g., an anti-cancer therapy provided herein (e.g., a VHL-targeted therapy or a PBRM1-targeted therapy), for example, as compared to an individual whose cancer does not exhibit the one or more mutations in a VHL gene or a PBRM1 gene. In some embodiments, the sample is a sample described herein. In some embodiments, the sample comprises cells from the cancer or is obtained from cells from the cancer, e.g., the ccRCC.

In some aspects, provided herein are methods of screening an individual having cancer, suspected of having cancer, being tested for cancer, being treated for cancer, or being tested for a susceptibility to cancer, e.g., a ccRCC. In some embodiments, the individual is being treated with an anti-cancer therapy, such as an anti-cancer therapy described herein. In some embodiments, the methods comprise acquiring knowledge of one or more mutations in a VHL gene or a PBRM1 gene in a sample from the individual. In some embodiments, the methods comprise detecting one or more mutations in a VHL gene or a PBRM1 gene in a sample from the individual. In some embodiments, responsive to acquiring knowledge of one or more mutations in a VHL gene or a PBRM1 gene in the sample, the individual is predicted to have increased risk of cancer recurrence, aggressive cancer, anti-cancer therapy resistance, or poor prognosis, for example, as compared to an individual whose cancer does not exhibit the one or more mutations in a VHL gene or a PBRM1 gene. In some embodiments, responsive to detecting one or more mutations in a VHL gene or a PBRM1 gene in the sample, the individual is predicted to have increased risk of cancer recurrence, aggressive cancer, anti-cancer therapy resistance, or poor prognosis, for example, as compared to an individual whose cancer does not exhibit one or more mutations in a VHL gene or a PBRM1 gene. In some embodiments, the methods further comprise providing a diagnosis or an assessment. In some embodiments, the diagnosis or assessment identifies the presence or absence of the one or more mutations in a VHL gene or a PBRM1 gene in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the one or more mutations in a VHL gene or a PBRM1 gene. In some embodiments, the sample is a sample described herein. In some embodiments, the sample comprises cells from the cancer or is obtained from cells from the cancer, e.g., the ccRCC.

In some embodiments, the methods further comprise selectively enriching for one or more nucleic acids comprising nucleotide sequences that comprise one or more mutations in a VHL gene or a PBRM1 gene to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise one or more mutations in a VHL gene or a PBRM1 gene to produce an enriched sample, e.g., using a reagent known in the art or provided herein, such as an antibody described herein.

In some aspects, provided herein are methods of diagnosing or assessing one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from an individual having a cancer, such as a ccRCC. In some embodiments, the methods comprise acquiring knowledge of the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, the methods comprise detecting one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual. In some embodiments, one or more mutations in one or more genes selected from CDKN2A, CDKN2B, VHL, PBRM1, SETD2, BAP1, TP53, PTEN, TERT, TSC1, PIK3CA, ATM, NF2, or MET are detected in a sample obtained from the individual using any method known in the art, such as one or more of the methods of detection of nucleic acid molecules or polypeptides described herein. In some embodiments, the methods further comprise providing a diagnosis or an assessment of the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the diagnosis or assessment identifies the presence or absence of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, the diagnosis or assessment identifies the cancer, such as a ccRCC provided herein, as likely to respond to an anti-cancer therapy, e.g., an anti-cancer therapy provided herein. In some embodiments, the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the cancer as likely to respond to an anti-cancer therapy, e.g., an anti-cancer therapy provided herein. In some embodiments, the diagnosis or assessment identifies the presence or absence of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, the diagnosis or assessment identifies the cancer, such as a ccRCC provided herein, as likely to respond to an anti-cancer therapy, e.g., an anti-cancer therapy provided herein. In some embodiments, the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample identifies the cancer as likely to respond to an anti-cancer therapy, e.g., an anti-cancer therapy provided herein. In some embodiments, the sample is a sample described herein. In some embodiments, the sample comprises cells from the cancer or is obtained from cells from the cancer, e.g., from a ccRCC. In some embodiments, the individual has a cancer, is suspected of having a cancer, is being tested for a cancer, is being treated for a cancer, or is being tested for a susceptibility to a cancer, e.g., a ccRCC.

In some aspects, provided herein are methods of diagnosing or assessing a cancer in an individual, e.g., a ccRCC. In some embodiments, the methods of diagnosing or assessing cancer comprise detecting one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual, e.g., a sample comprising cells from the cancer, e.g., a ccRCC. In some embodiments, the methods comprise detecting one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual using any method known in the art, such as one or more of the methods of detection of nucleic acid molecules or polypeptides described herein. In some embodiments, detection of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual identifies the cancer, e.g., the ccRCC, as likely to respond to an anti-cancer therapy, e.g., an anti-cancer therapy provided herein. In some embodiments, the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual identifies the cancer, e.g., the ccRCC, as likely to respond to an anti-cancer therapy, e.g., an anti-cancer therapy provided herein. In some embodiments, the methods further comprise providing a diagnosis or an assessment of the cancer, e.g., the ccRCC, or of one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the diagnosis or assessment identifies the cancer as likely to respond to an anti-cancer therapy, e.g., an anti-cancer therapy provided herein. In some embodiments, detection of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual identifies the cancer, e.g., the ccRCC, as likely to respond to an anti-cancer therapy, e.g., an anti-cancer therapy provided herein. In some embodiments, the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from the individual identifies the cancer, e.g., the ccRCC, as likely to respond to an anti-cancer therapy, e.g., an anti-cancer therapy provided herein. In some embodiments, the methods further comprise providing a diagnosis or an assessment of the cancer, e.g., the ccRCC, or of the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the diagnosis or assessment identifies the cancer as likely to respond to an anti-cancer therapy, e.g., an anti-cancer therapy provided herein. In some embodiments, the diagnosis or assessment identifies the presence or absence of the one or more mutations in one or more genes selected from CDKN2A, CDKN2B, VHL, PBRM1, SETD2, BAP1, TP53, PTEN, TERT, TSC1, PIK3CA, ATM, NF2, or MET in the sample.

In some aspects, provided herein are methods of predicting survival of an individual having a cancer, e.g., a cancer provided herein, e.g., a ccRCC. In some embodiments, the individual is being treated with an anti-cancer therapy, such as an anti-cancer therapy described herein. In some embodiments, the methods comprise acquiring knowledge of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from the individual. In some embodiments, the methods comprise detecting one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from the individual. In some embodiments, responsive to acquiring knowledge of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have longer survival after treatment with an anti-cancer therapy, e.g., an anti-cancer therapy provided herein, for example, as compared to an individual whose cancer does not exhibit the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, responsive to detecting one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have longer survival after treatment with an anti-cancer therapy, e.g., an anti-cancer therapy provided herein, for example, as compared to an individual whose cancer does not exhibit the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, responsive to acquiring knowledge of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have longer survival after treatment with an anti-cancer therapy, e.g., an anti-cancer therapy provided herein, for example, as compared to an individual whose cancer does not exhibit the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, responsive to detecting one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have longer survival after treatment with an anti-cancer therapy, e.g., an anti-cancer therapy provided herein, for example, as compared to an individual whose cancer does not exhibit the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the methods further comprise providing a diagnosis or an assessment. In some embodiments, the diagnosis or assessment identifies the presence or absence of the one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the sample is a sample described herein. In some embodiments, the sample comprises cells from the cancer or is obtained from cells from the cancer, e.g., the ccRCC.

In some aspects, provided herein are methods of screening an individual having cancer, suspected of having cancer, being tested for cancer, being treated for cancer, or being tested for a susceptibility to cancer, e.g., a ccRCC. In some embodiments, the individual is being treated with an anti-cancer therapy, such as an anti-cancer therapy described herein. In some embodiments, the methods comprise acquiring knowledge of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from the individual. In some embodiments, the methods comprise detecting one or more mutations in one or more genes selected from CDKN2A, CDKN2B, VHL, PBRM1, SETD2, BAP1, TP53, PTEN, TERT, TSC1, PIK3CA, ATM, NF2, or MET in a sample from the individual. In some embodiments, responsive to acquiring knowledge of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have increased risk of cancer recurrence, aggressive cancer, anti-cancer therapy resistance, or poor prognosis, for example, as compared to an individual whose cancer does not exhibit the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, responsive to detecting one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, the individual is predicted to have increased risk of cancer recurrence, aggressive cancer, anti-cancer therapy resistance, or poor prognosis, for example, as compared to an individual whose cancer does not exhibit the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the methods further comprise providing a diagnosis or an assessment. In some embodiments, the diagnosis or assessment identifies the presence or absence of the one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the sample is a sample described herein. In some embodiments, the sample comprises cells from the cancer or is obtained from cells from the cancer, e.g., the ccRCC.

In some embodiments, the methods further comprise selectively enriching for one or more nucleic acids comprising nucleotide sequences that comprise one or more mutations in one or more genes selected from CDKN2A, CDKN2B, VHL, PBRM1, SETD2, BAP1, TP53, PTEN, TERT, TSC1, PIK3CA, ATM, NF2, or MET to produce an enriched sample, e.g., using a reagent known in the art or provided herein, such as a bait, probe, or oligonucleotide described herein. In some embodiments, the methods further comprise selectively enriching for one or more polypeptides comprising amino acid sequences that comprise one or more mutations in one or more genes selected from CDKN2A, CDKN2B, VHL, PBRM1, SETD2, BAP1, TP53, PTEN, TERT, TSC1, PIK3CA, ATM, NF2, or MET to produce an enriched sample, e.g., using a reagent known in the art or provided herein, such as an antibody described herein.

In some embodiments of any of the methods provided herein, the methods further comprise acquiring knowledge of or detecting in a sample from an individual the absence of a deletion of an MTAP gene, or of a portion thereof. In some embodiments of any of the methods provided herein, the individual does not have a deletion of an MTAP gene, or of a portion thereof.

Reporting

In some embodiments, the methods provided herein comprise generating a report, and/or providing a report to party.

In some embodiments, a report according to the present disclosure comprises information about one or more of: one or more mutations in a VHL gene or a PBRM1 gene; a cancer of the disclosure (e.g., a ccRCC, e.g., comprising one or more mutations in a VHL gene or a PBRM1 gene); or a treatment, a therapy, or one or more treatment options (e.g., a VHL-targeted therapy or a PBRM1-targeted therapy) for an individual having a cancer, such as a cancer of the disclosure (e.g., a ccRCC, e.g., comprising one or more mutations in a VHL gene or a PBRM1 gene). In some embodiments, a report according to the present disclosure comprises information about one or more of: one or more mutations in a VHL gene; a cancer of the disclosure (e.g., a ccRCC, e.g., comprising one or more mutations in a VHL gene); or a treatment, a therapy, or one or more treatment options (e.g., a VHL-targeted therapy) for an individual having a cancer, such as a cancer of the disclosure (e.g., a ccRCC, e.g., comprising one or more mutations in a VHL gene). In some embodiments, a report according to the present disclosure comprises information about one or more of: one or more mutations in a PBRM1 gene; a cancer of the disclosure (e.g., a ccRCC, e.g., comprising one or more mutations in a PBRM1 gene); or a treatment, a therapy, or one or more treatment options (e.g., a PBRM1-targeted therapy) for an individual having a cancer, such as a cancer of the disclosure (e.g., a ccRCC, e.g., comprising one or more mutations in a PBRM1 gene). In some embodiments, a report according to the present disclosure further comprises information about the absence of a deletion of an MTAP gene, or of a portion thereof, in the sample. In some embodiments, a report according to the present disclosure comprises information about the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample.

In some embodiments, a report according to the present disclosure comprises information about the presence or absence of one or more mutations in a VHL gene or a PBRM1 gene in a sample obtained from an individual, such as an individual having a cancer, e.g., a cancer provided herein, e.g., a ccRCC. In some embodiments, a report according to the present disclosure further comprises information about the absence of a deletion of an MTAP gene, or of a portion thereof, in the sample. In some embodiments, a report according to the present disclosure comprises information about the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In one embodiment, a report according to the present disclosure indicates that one or more mutations in a VHL gene or a PBRM1 gene are present in a sample obtained from the individual. In one embodiment, a report according to the present disclosure indicates that a deletion of an MTAP gene, or of a portion thereof, is not present in the sample. In one embodiment, a report according to the present disclosure indicates that one or more mutations in one or more genes selected from CDKN2A, CDKN2B, VHL, PBRM1, SETD2, BAP1, TP53, PTEN, TERT, TSC1, PIK3CA, ATM, NF2, or MET are not present in the sample. In one embodiment, a report according to the present disclosure indicates that one or more mutations in one or more genes selected from CDKN2A, CDKN2B, VHL, PBRM1, SETD2, BAP1, TP53, PTEN, TERT, TSC1, PIK3CA, ATM, NF2, or MET are present in the sample. In one embodiment, a report according to the present disclosure indicates that one or more mutations in a VHL gene or a PBRM1 gene are not present in a sample obtained from the individual. In one embodiment, a report according to the present disclosure indicates that one or more mutations in a VHL gene or a PBRM1 gene have been detected in a sample obtained from the individual. In one embodiment, a report according to the present disclosure indicates that one or more mutations in a VHL gene or a PBRM1 gene have not been detected in a sample obtained from the individual. In one embodiment, a report according to the present disclosure indicates that a deletion of an MTAP gene, or of a portion thereof, has not been detected in the sample. In one embodiment, a report according to the present disclosure indicates that one or more mutations in one or more genes selected from CDKN2A, CDKN2B, VHL, PBRM1, SETD2, BAP1, TP53, PTEN, TERT, TSC1, PIK3CA, ATM, NF2, or MET have not been detected in the sample. In one embodiment, a report according to the present disclosure further indicates that one or more mutations in one or more genes selected from CDKN2A, CDKN2B, VHL, PBRM1, SETD2, BAP1, TP53, PTEN, TERT, TSC1, PIK3CA, ATM, NF2, or MET have been detected in the sample. In some embodiments, the report comprises an identifier for the individual from which the sample was obtained.

In some embodiments, the report includes information on the role of one or more mutations in a VHL gene or a PBRM1 gene, or of the corresponding wild type counterparts, in disease, such as in cancer, e.g., a ccRCC. In some embodiments, the report includes information on the role of the absence of a deletion of an MTAP gene, or of a portion thereof, in disease, such as in cancer, e.g., a ccRCC. In some embodiments, the report includes information on the role of one or more mutations in one or more genes selected from CDKN2A, CDKN2B, VHL, PBRM1, SETD2, BAP1, TP53, PTEN, TERT, TSC1, PIK3CA, ATM, NF2, or MET in disease, such as in cancer, e.g., a ccRCC. Such information can include one or more of: information on prognosis of a cancer, such as a cancer provided herein, e.g., comprising one or more mutations in a VHL gene or a PBRM1 gene, and/or not having a deletion of an MTAP gene, or of a portion thereof; information on resistance of a cancer, such as a cancer provided herein, e.g., a ccRCC, such as a ccRCC comprising one or more mutations in a VHL gene or a PBRM1 gene, and/or not having a deletion of an MTAP gene, or of a portion thereof, to one or more treatments; information on potential or suggested therapeutic options (e.g., such as an anti-cancer therapy provided herein, or a treatment selected or identified according to the methods provided herein, e.g., a VHL-targeted therapy or a PBRM1-targeted therapy); or information on therapeutic options that should be avoided. Such information can include one or more of: information on prognosis of a cancer, such as a cancer provided herein, e.g., comprising one or more mutations in one or more genes selected from CDKN2A, CDKN2B, VHL, PBRM1, SETD2, BAP1, TP53, PTEN, TERT, TSC1, PIK3CA, ATM, NF2, or MET; information on resistance of a cancer, such as a cancer provided herein, e.g., a ccRCC, such as a ccRCC comprising one or more mutations in one or more genes selected from CDKN2A, CDKN2B, VHL, PBRM1, SETD2, BAP1, TP53, PTEN, TERT, TSC1, PIK3CA, ATM, NF2, or MET, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein, e.g., a VHL-targeted therapy or a PBRM1-targeted therapy to an individual having a cancer, such as a cancer provided herein (e.g., a ccRCC), e.g., comprising one or more mutations in a VHL gene or a PBRM1 gene, and/or not having a deletion of an MTAP gene, or of a portion thereof, and identified in the report. In some embodiments, the report includes information or a recommendation on the administration of a treatment (e.g., an anti-cancer therapy provided herein, or a treatment selected or identified according to the methods provided herein, such as a VHL-targeted therapy or a PBRM1-targeted therapy). In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein (e.g., a ccRCC), e.g., comprising one or more mutations in one or more genes selected from CDKN2A, CDKN2B, VHL, PBRM1, SETD2, BAP1, TP53, PTEN, TERT, TSC1, PIK3CA, ATM, NF2, or MET, and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein (e.g., a ccRCC); detecting one or more mutations in a VHL gene or a PBRM1 gene in the sample, or acquiring knowledge of the presence of one or more mutations in a VHL gene or a PBRM1 gene in the sample; and generating a report. In some embodiments, the method comprises detecting the absence of a deletion of an MTAP gene, or of a portion thereof, in the sample, or acquiring knowledge of the absence of a deletion of an MTAP gene, or of a portion thereof, in the sample. In some embodiments, the method comprises detecting one or more mutations in one or more genes selected from CDKN2A, CDKN2B, VHL, PBRM1, SETD2, BAP1, TP53, PTEN, TERT, TSC1, PIK3CA, ATM, NF2, or MET in the sample, or acquiring knowledge of one or more mutations in one or more genes selected from CDKN2A, CDKN2B, VHL, PBRM1, SETD2, BAP1, TP53, PTEN, TERT, TSC1, PIK3CA, ATM, NF2, or MET in the sample. In some embodiments, a report generated according to the methods provided herein comprises one or more of: information about the presence or absence of one or more mutations in a VHL gene or a PBRM1 gene in the sample; an identifier for the individual from which the sample was obtained; information on the role of one or more mutations in a VHL gene or a PBRM1 gene, or the corresponding wild type counterparts, in disease (e.g., such as in cancer, e.g., a ccRCC); information on prognosis, resistance, or potential or suggested therapeutic options (such as an anti-cancer therapy provided herein, or a treatment selected or identified according to the methods provided herein, such as a VHL-targeted therapy or a PBRM1-targeted therapy); information on the likely effectiveness, acceptability, or the advisability of applying a therapeutic option (such as an anti-cancer therapy provided herein, or a treatment selected or identified according to the methods provided herein, e.g., a VHL-targeted therapy or a PBRM1-targeted therapy) to the individual; a recommendation or information on the administration of a treatment (such as an anti-cancer therapy provided herein, or a treatment selected or identified according to the methods provided herein, e.g., a VHL-targeted therapy or a PBRM1-targeted therapy); or a recommendation or information on the dosage or treatment regimen of a treatment (such as an anti-cancer therapy provided herein, or a treatment selected or identified according to the methods provided herein, e.g., a VHL-targeted therapy or a PBRM1-targeted therapy), e.g., in combination with other treatments (e.g., a second therapeutic agent). In some embodiments, a report generated according to the methods provided herein comprises one or more of: information about the presence or absence one or more mutations in one or more genes selected from CDKN2A, CDKN2B, VHL, PBRM1, SETD2, BAP1, TP53, PTEN, TERT, TSC1, PIK3CA, ATM, NF2, or MET in the sample; an identifier for the individual from which the sample was obtained; information on the role of one or more mutations in one or more genes selected from CDKN2A, CDKN2B, VHL, PBRM1, SETD2, BAP1, TP53, PTEN, TERT, TSC1, PIK3CA, ATM, NF2, or MET, or the corresponding wild type counterparts, in disease (e.g., such as in cancer, e.g., a ccRCC); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, a report generated according to the methods provided herein further comprises information about the absence of a deletion of an MTAP gene, or of a portion thereof in the sample. In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., a ccRCC, such as a ccRCC comprising one or more mutations in a VHL gene or a PBRM1 gene, and/or optionally not having a deletion of an MTAP gene, or of a portion thereof), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting one or more mutations in a VHL gene or a PBRM1 gene, and/or optionally the absence of a deletion of an MTAP gene, or of a portion thereof, in a sample obtained from an individual (e.g., an individual having a cancer, e.g., a ccRCC). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of one or more mutations in a VHL gene or a PBRM1 gene, and/or optionally of a deletion of an MTAP gene, or of a portion thereof, in a sample obtained from an individual (e.g., an individual having a cancer). The report may be provided to an individual or a patient (e.g., an individual or a patient with a cancer, such as a cancer provided herein, e.g., a ccRCC, such as a ccRCC comprising one or more mutations in one or more genes selected from CDKN2A, CDKN2B, VHL, PBRM1, SETD2, BAP1, TP53, PTEN, TERT, TSC1, PIK3CA, ATM, NF2, or MET), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual (e.g., an individual having a cancer, e.g., a ccRCC). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of one or more mutations in one or more genes selected from CDKN2A, CDKN2B, VHL, PBRM1, SETD2, BAP1, TP53, PTEN, TERT, TSC1, PIK3CA, ATM, NF2, or MET in a sample obtained from an individual (e.g., an individual having a cancer).

### Gene Alterations

In some embodiments, the methods provided herein comprise acquiring knowledge of or detecting one or more gene alterations (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) in a sample from an individual, e.g., having a UCB, a NSCLC, a srcRCC or a ccRCC. In some embodiments, acquiring knowledge of one or more gene alterations (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) in a sample, comprises detecting the one or more gene alterations in the sample. In some embodiments, the methods provided herein comprise detecting one or more gene alterations (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) in a sample from an individual, e.g., having a UCB, a NSCLC, a srcRCC or a ccRCC.

In some embodiments, the one or more gene alterations comprise a deletion of an MTAP gene, CDKN2A gene, or CDKN2B gene, or portions thereof. In some embodiments, the deletion comprises a deletion of the entire gene or of a portion of the gene. In some embodiments, the deletion of the entire gene or of a portion of the gene result in a loss of function of the gene. In some embodiments, the deletion of an MTAP gene comprises a deletion of the entire MTAP gene or of a portion of the MTAP gene. In some embodiments, the deletion of the entire MTAP gene or of a portion of the MTAP gene result in a loss of function of the MTAP gene. In some embodiments, the deletion of a CDKN2A gene comprises a deletion of the entire CDKN2A gene or of a portion of the CDKN2A gene. In some embodiments, the deletion of the entire CDKN2A gene or of a portion of the CDKN2A gene result in a loss of function of the CDKN2A gene. In some embodiments, the deletion of a CDKN2B gene comprises a deletion of the entire CDKN2B gene or of a portion of the CDKN2B gene. In some embodiments, the deletion of the entire CDKN2B gene or of a portion of the CDKN2B gene result in a loss of function of the CDKN2B gene. In some embodiments, the deletion of an MTAP gene is a homozygous deletion or a heterozygous deletion. In some embodiments, the deletion of an CDKN2A gene is a homozygous deletion or a heterozygous deletion. In some embodiments, the deletion of an CDKN2B gene is a homozygous deletion or a heterozygous deletion.

In some embodiments, the one or more gene alterations comprise one or more mutations in one or more of an FGFR3 gene, a PTEN gene, an RB1 gene, an EGFR gene, a SMARCA4 gene, a KRAS gene, a TP53 gene, an NF2 gene, a VHL gene, or a PBRM1 gene. In some embodiments, the one or more mutations in the KRAS gene result in a G12C amino acid substitution in a KRAS polypeptide encoded by the KRAS gene. In some embodiments, the one or more gene alterations comprise one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the one or more gene alterations, e.g., in a UCB comprising an intact MTAP gene, comprise one or more mutations in one or more of TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the one or more gene alterations, e.g., in a UCB comprising a deletion of an MTAP gene or of a portion thereof, comprise one or more mutations in one or more of MTAP, CDKN2A, CDKN2B, TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the one or more gene alterations, e.g., in a NSCLC comprising an intact MTAP gene, comprise one or more mutations in one or more of CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the one or more gene alterations, e.g., in a NSCLC comprising a deletion of an MTAP gene or of a portion thereof, comprise one or more mutations in one or more of CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the one or more gene alterations, e.g., in a ccRCC comprising an intact MTAP gene, comprise one or more mutations in one or more of CDKN2A, CDKN2B, VHL, PBRM1, SETD2, BAP1, TP53, PTEN, TERT, TSC1, PIK3CA, ATM, NF2, or MET. In some embodiments, the one or more gene alterations, e.g., in a srcRCC or a ccRCC comprising a deletion of an MTAP gene or of a portion thereof, comprise one or more mutations in one or more of CDKN2A, CDKN2B, VHL, PBRM1, SETD2, BAP1, TP53, PTEN, TERT, TSC1, PIK3CA, ATM, NF2, or MET. In some embodiments, the one or more mutations comprise one or more of a substitution of one or more nucleotides, an insertion of one or more nucleotides, or a deletion of one or more nucleotides. In some embodiments, the one or more mutations comprise one or more of a genomic rearrangement, an alteration in a promoter, a gene fusion, or a copy number alteration. In some embodiments, the one or more mutations comprise a gene copy number alteration. In some embodiments, the one or more mutations comprise a gene amplification. In some embodiments, the one or more mutations comprise a gene deletion, e.g., a deletion of the entire gene or of a portion of the gene. In some embodiments, the one or more mutations comprise a point mutation. In some embodiments, the one or more mutations comprise a single nucleotide polymorphism. In some embodiments, the one or more mutations comprise one or more mutations in an exon and/or an intron of the gene. In some embodiments, the one or more mutations comprise a non-synonymous mutation. In some embodiments, the one or more mutations comprise a missense mutation. In some embodiments, the one or more mutations comprise a nonsense mutation. In some embodiments, the one or more mutations comprise a gain-of-function mutation, e.g., an activating mutation. In some embodiments, the one or more mutations comprise a loss-of-function mutation, e.g., an inactivating mutation. In some embodiments, the one or more mutations result in a frameshift. In some embodiments, the one or more mutations result in a premature stop codon. In some embodiments, the one or more mutations comprise a functional alteration. In some embodiments, the one or more mutations comprise a mutation that alters the function of the polypeptide or protein encoded by the gene. In some embodiments, the one or more mutations comprise a complex insertion. In some embodiments, the one or more mutations comprise a complex deletion. In some embodiments, the one or more mutations comprise a mutation in a splice site. In some embodiments, the one or more mutations alter the splicing of an mRNA molecule encoded by the gene. In some embodiments, the one or more mutations comprise an insertion of one or more nucleotides. In some embodiments, the insertion comprises an insertion of between about 1 and about 5 nucleotides, between about 5 and about 10 nucleotides, between about 10 and about 20 nucleotides, between about 20 and about 30 nucleotides, between about 30 and about 40 nucleotides, or between about 40 and about 50 nucleotides. In some embodiments, the insertion comprises an insertion of between about 50 and about 100 nucleotides, between about 100 and about 200 nucleotides, between about 200 and about 300 nucleotides, between about 300 and 400 nucleotides, between about 400 and about 500 nucleotides, between about 500 and about 600 nucleotides, between about 600 and about 700 nucleotides, between about 700 and about 800 nucleotides, between about 800 and about 900 nucleotides, or between about 900 and about 1000 nucleotides. In some embodiments, the insertion comprises an insertion of between about 1000 and about 1500 nucleotides, between about 1500 and about 2000 nucleotides, between about 2000 and about 2500 nucleotides, between about 2500 and about 3000 nucleotides, between about 3000 and about 3500 nucleotides, between about 3500 and about 4000 nucleotides, between about 4000 and about 4500 nucleotides, between about 4500 and about 5000 nucleotides, between about 5000 and about 5500 nucleotides, between about 5500 and about 6000 nucleotides, between about 6000 and about 6500 nucleotides, between about 6500 and about 7000 nucleotides, between about 7000 and about 7500 nucleotides, between about 7500 and about 8000 nucleotides, between about 8000 and about 8500 nucleotides, between about 8500 and about 9000 nucleotides, between about 9000 and about 9500 nucleotides, or between about 9500 and about 10000 nucleotides. In some embodiments, the one or more mutations comprise a deletion of one or more nucleotides. In some embodiments, the deletion comprises a deletion of between about 1 and about 5 nucleotides, between about 5 and about 10 nucleotides, between about 10 and about 20 nucleotides, between about 20 and about 30 nucleotides, between about 30 and about 40 nucleotides, or between about 40 and about 50 nucleotides. In some embodiments, the deletion comprises a deletion of between about 50 and about 100 nucleotides, between about 100 and about 200 nucleotides, between about 200 and about 300 nucleotides, between about 300 and 400 nucleotides, between about 400 and about 500 nucleotides, between about 500 and about 600 nucleotides, between about 600 and about 700 nucleotides, between about 700 and about 800 nucleotides, between about 800 and about 900 nucleotides, or between about 900 and about 1000 nucleotides. In some embodiments, the deletion comprises a deletion of between about 1000 and about 1500 nucleotides, between about 1500 and about 2000 nucleotides, between about 2000 and about 2500 nucleotides, between about 2500 and about 3000 nucleotides, between about 3000 and about 3500 nucleotides, between about 3500 and about 4000 nucleotides, between about 4000 and about 4500 nucleotides, between about 4500 and about 5000 nucleotides, between about 5000 and about 5500 nucleotides, between about 5500 and about 6000 nucleotides, between about 6000 and about 6500 nucleotides, between about 6500 and about 7000 nucleotides, between about 7000 and about 7500 nucleotides, between about 7500 and about 8000 nucleotides, between about 8000 and about 8500 nucleotides, between about 8500 and about 9000 nucleotides, between about 9000 and about 9500 nucleotides, or between about 9500 and about 10000 nucleotides. In some embodiments, the one or more mutations result in a substitution, insertion, or deletion of one or more amino acid residues in a polypeptide or a protein encoded by the gene. In some embodiments, the one or more mutations result in a substitution of one or more amino acid residues in a polypeptide or a protein encoded by the gene. In some embodiments, the one or more mutations result in a deletion of one or more amino acid residues in a polypeptide or a protein encoded by the gene. In some embodiments, the one or more mutations result in an insertion of one or more amino acid residues in a polypeptide or a protein encoded by the gene.

An exemplary nucleic acid sequence of an AKT2 gene is available as Transcript ID NM_001626, available at the website: www[dot]ncbi[dot]nlm[dot]nih[dot]gov/nuccore/NM_001626. An exemplary nucleic acid sequence of an APC gene is available as Transcript ID NM_000038, available at the website: www[dot]ncbi[dot]nlm[dot]nih[dot]gov/nuccore/NM_000038. An exemplary nucleic acid sequence of an ARID1A gene is available as Transcript ID NM_006015, available at the website: www[dot]ncbi[dot]nlm[dot]nih[dot]gov/nuccore/NM_006015. An exemplary nucleic acid sequence of an ATM gene is available as Transcript ID NM_000051, available at the website: www[dot]ncbi[dot]nlm[dot]nih[dot]gov/nuccore/NM_000051. An exemplary nucleic acid sequence of a BAP1 gene is available as Transcript ID NM_004656, available at the website: www[dot]ncbi[dot]nlm[dot]nih[dot]gov/nuccore/NM_004656. An exemplary nucleic acid sequence of a BCL2L1 gene is available as Transcript ID NM_138578, available at the website: www[dot]ncbi[dot]nlm[dot]nih[dot]gov/nuccore/NM_138578. An exemplary nucleic acid sequence of a BRAF gene is available as Transcript ID NM_004333, available at the website: www[dot]ncbi[dot]nlm[dot]nih[dot]gov/nuccore/NM_004333. An exemplary nucleic acid sequence of a BRCA2 gene is available as Transcript ID NM_000059, available at the website: www[dot]ncbi[dot]nlm[dot]nih[dot]gov/nuccore/NM_000059. An exemplary nucleic acid sequence of a CCND1 gene is available as Transcript ID NM_053056, available at the website: www[dot]ncbi[dot]nlm[dot]nih[dot]gov/nuccore/NM_053056. An exemplary nucleic acid sequence of a CCNE1 gene is available as Transcript ID NM_001238, available at the website: www[dot]ncbi[dot]nlm[dot]nih[dot]gov/nuccore/NM_001238. An exemplary nucleic acid sequence of a CDH1 gene is available as Transcript ID NM_004360, available at the website: www[dot]ncbi[dot]nlm[dot]nih[dot]gov/nuccore/NM_004360. An exemplary nucleic acid sequence of a CDKN2A gene is available as Transcript ID NM_000077, available at the website: www[dot]ncbi[dot]nlm[dot]nih[dot]gov/nuccore/NM_000077. An exemplary nucleic acid sequence of a CDKN2B gene is available as Transcript ID NM_004936, available at the website: www[dot]ncbi[dot]nlm[dot]nih[dot]gov/nuccore/NM_004936. An exemplary nucleic acid sequence of a CHEK2 gene is available as Transcript ID NM_007194, available at the website: www[dot]ncbi[dot]nlm[dot]nih[dot]gov/nuccore/NM_007194. An exemplary nucleic acid sequence of a CREBBP gene is available as Transcript ID NM_004380, available at the website: www[dot]ncbi[dot]nlm[dot]nih[dot]gov/nuccore/NM_004380. An exemplary nucleic acid sequence of an DNMT3A gene is available as Transcript ID NM_022552, available at the website: www[dot]ncbi[dot]nlm[dot]nih[dot]gov/nuccore/NM_022552. An exemplary nucleic acid sequence of an EGFR gene is available as Transcript ID NM_005228, available at the website: www[dot]ncbi[dot]nlm[dot]nih[dot]gov/nuccore/NM_005228. An exemplary nucleic acid sequence of an EP300 gene is available as Transcript ID NM_001429, available at the website: www[dot]ncbi[dot]nlm[dot]nih[dot]gov/nuccore/NM_001429. An exemplary nucleic acid sequence of an EPHA3 gene is available as Transcript ID NM_005233, available at the website: www[dot]ncbi[dot]nlm[dot]nih[dot]gov/nuccore/NM_005233. An exemplary nucleic acid sequence of an ERBB2 gene is available as Transcript ID NM_004448, available at the website: www[dot]ncbi[dot]nlm[dot]nih[dot]gov/nuccore/NM_004448. An exemplary nucleic acid sequence of an ERBB3 gene is available as Transcript ID NM_001982, available at the website: www[dot]ncbi[dot]nlm[dot]nih[dot]gov/nuccore/NM_001982. An exemplary nucleic acid sequence of an FBXW7 gene is available as Transcript ID NM_033632, available at the website: www[dot]ncbi[dot]nlm[dot]nih[dot]gov/nuccore/NM_033632. An exemplary nucleic acid sequence of an FGF10 gene is available as Transcript ID NM_004465, available at the website: www[dot]ncbi[dot]nlm[dot]nih[dot]gov/nuccore/NM_004465. An exemplary nucleic acid sequence of an FGF19 gene is available as Transcript ID NM_005117, available at the website: www[dot]ncbi[dot]nlm[dot]nih[dot]gov/nuccore/NM_005117. An exemplary nucleic acid sequence of an FGF3 gene is available as Transcript ID NM_005247, available at the website: www[dot]ncbi[dot]nlm[dot]nih[dot]gov/nuccore/NM_005247. An exemplary nucleic acid sequence of an FGF4 gene is available as Transcript ID NM_002007, available at the website: www[dot]ncbi[dot]nlm[dot]nih[dot]gov/nuccore/NM_002007. An exemplary nucleic acid sequence of an FGFR1 gene is available as Transcript ID NM_023110, available at the website: www[dot]ncbi[dot]nlm[dot]nih[dot]gov/nuccore/NM_023110. An exemplary nucleic acid sequence of an FGFR3 gene is available as Transcript ID NM_000142, available at the website: www[dot]ncbi[dot]nlm[dot]nih[dot]gov/nuccore/NM_000142. An exemplary nucleic acid sequence of an HRAS gene is available as Transcript ID NM_005343, available at the website: www[dot]ncbi[dot]nlm[dot]nih[dot]gov/nuccore/NM_005343. An exemplary nucleic acid sequence of a KDM6A gene is available as Transcript ID NM_021140, available at the website: www[dot]ncbi[dot]nlm[dot]nih[dot]gov/nuccore/NM_021140. An exemplary nucleic acid sequence of a KMT2D gene is available as Transcript ID NM_003482 available at the website: www[dot]ncbi[dot]nlm[dot]nih[dot]gov/nuccore/NM_003482. An exemplary nucleic acid sequence of a KRAS gene is available as Transcript ID NM_004985, available at the website: www[dot]ncbi[dot]nlm[dot]nih[dot]gov/nuccore/NM_004985. An exemplary nucleic acid sequence of an MCL1 gene is available as Transcript ID NM_182763, available at the website: www[dot]ncbi[dot]nlm[dot]nih[dot]gov/nuccore/NM_182763. An exemplary nucleic acid sequence of an CDKN1A gene is available as Transcript ID NM_000389, available at the website: www[dot]ncbi[dot]nlm[dot]nih[dot]gov/nuccore/NM_000389. An exemplary nucleic acid sequence of an MDM2 gene is available as Transcript ID NM_002392, available at the website: www[dot]ncbi[dot]nlm[dot]nih[dot]gov/nuccore/NM_002392. An exemplary nucleic acid sequence of an MTAP gene is available as Transcript ID NM_002451, available at the website: www[dot]ncbi[dot]nlm[dot]nih[dot]gov/nuccore/NM_002451. An exemplary nucleic acid sequence of an MUTYH gene is available as Transcript ID NM_001048171, available at the website: www[dot]ncbi[dot]nlm[dot]nih[dot]gov/nuccore/NM_001048171. An exemplary nucleic acid sequence of a MYC gene is available as Transcript ID NM_002467, available at the website: www[dot]ncbi[dot]nlm[dot]nih[dot]gov/nuccore/NM_002467. An exemplary nucleic acid sequence of a MYCL gene is available as Transcript ID NM_005376, available at the website: www[dot]ncbi[dot]nlm[dot]nih[dot]gov/nuccore/NM_005376. An exemplary nucleic acid sequence of an NF1 gene is available as Transcript ID NM_001042492, available at the website: www[dot]ncbi[dot]nlm[dot]nih[dot]gov/nuccore/NM_001042492. An exemplary nucleic acid sequence of an NFE2L2 gene is available as Transcript ID NM_006164, available at the website: www[dot]ncbi[dot]nlm[dot]nih[dot]gov/nuccore/NM_006164. An exemplary nucleic acid sequence of a NOTCH3 gene is available as Transcript ID NM_000435, available at the website: www[dot]ncbi[dot]nlm[dot]nih[dot]gov/nuccore/NM_000435. An exemplary nucleic acid sequence of an NSD3 gene is available as Transcript ID NM_017778, available at the website: www[dot]ncbi[dot]nlm[dot]nih[dot]gov/nuccore/NM_017778; or as Transcript ID NM_023034, available at the website: www[dot]ncbi[dot]nlm[dot]nih[dot]gov/nuccore/NM_023034. An exemplary nucleic acid sequence of a PBRM1 gene is available as Transcript ID NM_018313, available at the website: www[dot]ncbi[dot]nlm[dot]nih[dot]gov/nuccore/NM_018313. An exemplary nucleic acid sequence of a PIK3CA gene is available as Transcript ID NM_006218, available at the website: www[dot]ncbi[dot]nlm[dot]nih[dot]gov/nuccore/NM_006218. An exemplary nucleic acid sequence of a PTEN gene is available as Transcript ID NM_000314, available at the website: www[dot]ncbi[dot]nlm[dot]nih[dot]gov/nuccore/NM_000314. An exemplary nucleic acid sequence of a RAD21 gene is available as Transcript ID NM_006265, available at the website: www[dot]ncbi[dot]nlm[dot]nih[dot]gov/nuccore/NM_006265. An exemplary nucleic acid sequence of a RAF1 gene is available as Transcript ID NM_002880, available at the website: www[dot]ncbi[dot]nlm[dot]nih[dot]gov/nuccore/NM_002880. An exemplary nucleic acid sequence of an RB1 gene is available as Transcript ID NM_000321, available at the website: www[dot]ncbi[dot]nlm[dot]nih[dot]gov/nuccore/NM_000321. An exemplary nucleic acid sequence of an RBM10 gene is available as Transcript ID NM_005676, available at the website: www[dot]ncbi[dot]nlm[dot]nih[dot]gov/nuccore/NM_005676. An exemplary nucleic acid sequence of a RICTOR gene is available as Transcript ID NM_152756, available at the website: www[dot]ncbi[dot]nlm[dot]nih[dot]gov/nuccore/NM_152756. An exemplary nucleic acid sequence of an SF3B1 gene is available as Transcript ID NM_012433, available at the website: www[dot]ncbi[dot]nlm[dot]nih[dot]gov/nuccore/NM_012433. An exemplary nucleic acid sequence of a SMARCA4 gene is available as Transcript ID NM_003072, available at the website: www[dot]ncbi[dot]nlm[dot]nih[dot]gov/nuccore/NM_003072. An exemplary nucleic acid sequence of a STAG2 gene is available as Transcript ID NM_006603, available at the website: www[dot]ncbi[dot]nlm[dot]nih[dot]gov/nuccore/NM_006603. An exemplary nucleic acid sequence of a TERT gene is available as Transcript ID NM_198253, available at the website: www[dot]ncbi[dot]nlm[dot]nih[dot]gov/nuccore/NM_198253. An exemplary nucleic acid sequence of a TET2 gene is available as Transcript ID NM_001127208, available at the website: www[dot]ncbi[dot]nlm[dot]nih[dot]gov/nuccore/NM_001127208. An exemplary nucleic acid sequence of a TP53 gene is available as Transcript ID NM_000546, available at the website: www[dot]ncbi[dot]nlm[dot]nih[dot]gov/nuccore/NM_000546. An exemplary nucleic acid sequence of a TSC1 gene is available as Transcript ID NM_000368, available at the website: www[dot]ncbi[dot]nlm[dot]nih[dot]gov/nuccore/NM_000368. An exemplary nucleic acid sequence of a ZNF703 gene is available as Transcript ID NM_025069, available at the website: www[dot]ncbi[dot]nlm[dot]nih[dot]gov/nuccore/NM_025069. An exemplary nucleic acid sequence of an NF2 gene is available as Transcript ID NM_000268, available at the website: www[dot]ncbi[dot]nlm[dot]nih[dot]gov/nuccore/NM_000268. An exemplary nucleic acid sequence of a SETD2 gene is available as Transcript ID NM_014159, available at the website: www[dot]ncbi[dot]nlm[dot]nih[dot]gov/nuccore/NM_014159. An exemplary nucleic acid sequence of a VHL gene is available as Transcript ID NM_000551, available at the website: www[dot]ncbi[dot]nlm[dot]nih[dot]gov/nuccore/NM_000551. An exemplary nucleic acid sequence of a MET gene is available as Transcript ID NM_000245, available at the website: www[dot]ncbi[dot]nlm[dot]nih[dot]gov/nuccore/NM_000245. An exemplary nucleic acid sequence of a KEAP1 gene is available as Transcript ID NM_012289, available at the website: www[dot]ncbi[dot]nlm[dot]nih[dot]gov/nuccore/NM_012289. An exemplary nucleic acid sequence of a STK11 gene is available as Transcript ID NM_000455, available at the website: www[dot]ncbi[dot]nlm[dot]nih[dot]gov/nuccore/NM_000455. An exemplary nucleic acid sequence of a NTRK1 gene is available as Transcript ID NM_002529, available at the website: www[dot]ncbi[dot]nlm[dot]nih[dot]gov/nuccore/NM_002529. An exemplary nucleic acid sequence of a ROS1 gene is available as Transcript ID NM_002944, available at the website: www[dot]ncbi[dot]nlm[dot]nih[dot]gov/nuccore/NM_002944. An exemplary nucleic acid sequence of a ALK gene is available as Transcript ID NM_004304, available at the website: www[dot]ncbi[dot]nlm[dot]nih[dot]gov/nuccore/NM_004304. An exemplary nucleic acid sequence of a BRCA1 gene is available as Transcript ID NM_007294, available at the website: www[dot]ncbi[dot]nlm[dot]nih[dot]gov/nuccore/NM_007294.

Detection of Gene Alterations

In some embodiments, the methods provided herein comprise detecting one or more gene alterations, such as a deletion of one or more genes, or of a portion thereof (e.g., a deletion of an MTAP gene, or of a portion thereof; a deletion of a CDKN2A gene, or of a portion thereof; or a deletion of a CDKN2B gene, or of a portion thereof), or one or more mutations in one or more genes (e.g., in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703).

In some embodiments, the one or more gene alterations (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) are detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety.

In Situ Hybridization Methods

In some embodiments, one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) are detected using an in situ hybridization method, such as a fluorescence in situ hybridization (FISH) method.

In some embodiments, FISH analysis is used to identify the chromosomal rearrangement resulting in the mutations as described herein. In some embodiments, FISH analysis is used to identify an RNA molecule comprising one or more gene alterations described herein (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH.

Array-Based Methods

In some embodiments, one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) are detected using an array-based method, such as array-based comparative genomic hybridization (CGH) methods. In array-based CGH methods, a first sample of nucleic acids (e.g., from a sample, such as from a tumor) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor) is labeled and hybridized to a second array (with identical content) and absolute signals are read. Copy number differences are calculated based on absolute signals from the two arrays.

Amplification-Based Methods

In some embodiments, one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) are detected using an amplification-based method. As is known in the art, in such amplification-based methods, a sample of nucleic acids, such as a sample obtained from an individual or from a tumor, is used as a template in an amplification reaction (e.g., Polymerase Chain Reaction (PCR)) using one or more oligonucleotides or primers, e.g., such as one or more oligonucleotides or primers provided herein. The presence of one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

Sequencing

In some embodiments, one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) are detected using a sequencing method. Any method of sequencing known in the art can be used to detect one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). Exemplary sequencing methods that may be used to detect one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) include those based on techniques developed by Maxam and Gilbert or Sanger. Automated sequencing procedures may also be used, e.g., including sequencing by mass spectrometry.

In some embodiments, one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) are detected using hybrid capture-based sequencing (hybrid capture-based next-generation sequencing (NGS)), e.g., using adaptor ligation-based libraries. See, e.g., Frampton, G. M. et al. (2013) Nat. Biotech. 31:1023-1031. In some embodiments, one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) are detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments of any of the methods provided herein, the methods comprise providing a sample from an individual having a UCB, a NSCLC, a srcRCC or a ccRCC, wherein the sample comprises one or more nucleic acids. In some embodiments, the methods further comprise preparing a nucleic acid sequencing library from the one or more nucleic acids in the sample. Methods for the preparation of nucleic acid sequencing libraries, e.g., suitable for any of the sequencing methods described herein (e.g., next generation sequencing), are known in the art. In some embodiments, the sequencing library is prepared as described in Frampton et al (2013) Nat Biotechnol, 31:1023-1031. In some embodiments, the library is amplified. Methods for amplifying nucleic acid libraries (e.g., including the steps of adding sample indexes and/or barcodes) are known in the art. In some embodiments, the sequencing library is amplified using a polymerase chain reaction (PCR). In some embodiments, the sequencing library is amplified as described in Frampton et al (2013) Nat Biotechnol, 31:1023-1031. In some embodiments, the methods further comprise selectively enriching for one or more nucleic acids (e.g., one or more nucleic acids comprising one or more gene alterations described herein) to produce an enriched sample. In some embodiments, the selectively enriching comprises combining a bait, such as a bait described herein, with a sample (e.g., the library), thereby hybridizing the bait to the one or more nucleic acids in the sample (e.g., in the library), and producing nucleic acid hybrids; and isolating the nucleic acid hybrids to produce an enriched sample. In some embodiments, the selectively enriching is performed as described in Frampton et al (2013) Nat Biotechnol, 31:1023-1031. In some embodiments, the methods further comprise amplifying, e.g. using PCR, the nucleic acids in the enriched sample. In some embodiments, the methods further comprise sequencing the enriched sample, thereby producing a plurality of sequencing reads. In some embodiments, the sequencing is performed using any method for sequencing known in the art or provided herein. In some embodiments, the sequencing is performed using an Illumina sequencer. In some embodiments, the sequencing is performed as described in Frampton et al (2013) Nat Biotechnol, 31:1023-1031. In some embodiments, the methods further comprise analyzing the plurality of sequencing reads, e.g., for the presence of one or more gene alterations described herein. In some embodiments, the analyzing step comprises aligning the plurality of sequencing reads to the human genome, e.g., to human genome version hg19, e.g., using any suitable methods, such as a BWA aligner. In some embodiments, the analyzing step further comprises removing PCR duplicate reads, and/or collecting sequence metrics (e.g., using Picard 1.47 and/or Samtools). In some embodiments, the analyzing step comprises performing local alignment optimization, e.g., using GATK. In some embodiments, the analyzing step further comprises variant calling. In some embodiments, the analyzing step comprises detecting base substitutions, e.g., using a Bayesian methodology. In some embodiments, the analyzing step comprises detecting indels, e.g., using the Bruijn approach. In some embodiments, the analyzing step comprises detecting copy number alterations, e.g., using comparative genomic hybridization-like methods. In some embodiments, the analyzing step comprises detecting genomic rearrangements and/or gene fusions, e.g., by analyzing chimeric read pairs. In some embodiments, the analyzing step is performed as described in Frampton et al (2013) Nat Biotechnol, 31:1023-1031. In some embodiments, the methods further comprise detecting, based on the analyzing step, one or more gene alterations described herein.

Detection Reagents

In some aspects, provided herein are reagents for detecting one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid, e.g., a nucleic acid that comprises one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment or portion thereof.

Baits

Provided herein are baits suitable for the detection of one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703).

In some embodiments, the bait comprises a capture nucleic acid molecule configured to hybridize to a target nucleic acid molecule comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment or portion thereof. In some embodiments, the bait comprises a capture nucleic acid molecule configured to hybridize to a target nucleic acid molecule, e.g., a target nucleic acid molecule comprising nucleotide sequences of one or more genes selected from an MTAP gene, a CDKN2A gene, a CDKN2B gene, an FGFR3 gene, a PTEN gene, an RB1 gene, an EGFR gene, a SMARCA4 gene, a KRAS gene, a TP53 gene, an NF2 gene, a VHL gene, or a PBRM1 gene. In some embodiments, the capture nucleic acid molecule is configured to hybridize to a nucleic acid molecule encoding one or more genes selected from an MTAP gene, a CDKN2A gene, a CDKN2B gene, an FGFR3 gene, a PTEN gene, an RB1 gene, an EGFR gene, a SMARCA4 gene, a KRAS gene, a TP53 gene, an NF2 gene, a VHL gene, or a PBRM1 gene. In some embodiments, the one or more mutations in the KRAS gene result in a G12C amino acid substitution in a KRAS polypeptide encoded by the KRAS gene. In some embodiments, the bait comprises a capture nucleic acid molecule configured to hybridize to a target nucleic acid molecule, e.g., a target nucleic acid molecule comprising nucleotide sequences of one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the capture nucleic acid molecule is configured to hybridize to a nucleic acid molecule encoding one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the capture nucleic acid molecule is configured to hybridize to a fragment of a nucleic acid molecule comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises a nucleic acid sequence configured to hybridize to a nucleic acid molecule encoding an alteration described herein, e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703, e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides.

In some embodiments, the capture nucleic acid molecule is between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule is about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule is between about 5 and about 25 nucleotides in length, between about 5 and about 300 nucleotides in length, between about 100 and about 300 nucleotides in length, between about 130 and about 230 nucleotides in length, or between about 150 and about 200 nucleotides in length.

In some embodiments, the gene alteration is a gene fusion or a rearrangement. In some embodiments, the capture nucleic acid molecule is configured to hybridize to a breakpoint of a gene fusion or a rearrangement, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint. In some embodiments, the capture nucleic acid molecule is configured to hybridize to a nucleotide sequence in an intron or an exon of a first gene of a gene fusion or rearrangement, and/or to a nucleotide sequence in an intron or an exon of a second gene of a gene fusion or rearrangement, and/or to a nucleotide sequence in a breakpoint of a gene fusion or rearrangement (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid molecule is configured to hybridize to a breakpoint joining an intron or a exon of first gene and an intron or an exon of a second gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label or a tag. In some embodiments, the label or tag is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag, e.g., that allows capture and isolation of a hybrid formed by a bait and a nucleic acid hybridized to the bait. In some embodiments, the affinity tag is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

In some embodiments, the bait hybridizes to a nucleotide sequence encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) and a sequence on either side of the alteration (e.g., any of 1, 2, 3, 4, 5, 6, 7, 8, 9, or 10 nucleotides on either side of the alteration, or any of between about 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the alteration).

The baits described herein can be used for selection of exons and short target sequences.

In some embodiments, a bait of the disclosure distinguishes a nucleic acid, e.g., a genomic or transcribed nucleic acid, e.g., a cDNA or RNA, comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), from a reference nucleotide sequence, e.g., a nucleotide sequence not having the one or more gene alterations.

Probes

Also provided herein are probes, e.g., nucleic acid molecules, suitable for the detection of one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In some embodiments, a probe provided herein comprises a nucleic acid sequence configured to hybridize to a target nucleic acid molecule comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment or portion thereof. In some embodiments, a probe provided herein comprises a nucleic acid sequence configured to hybridize to a target nucleic acid molecule, e.g., a target nucleic acid molecule comprising nucleotide sequences of one or more genes selected from an MTAP gene, a CDKN2A gene, a CDKN2B gene, an FGFR3 gene, a PTEN gene, an RB1 gene, an EGFR gene, a SMARCA4 gene, a KRAS gene, a TP53 gene, an NF2 gene, a VHL gene, or a PBRM1 gene. In some embodiments, the probe comprises a nucleic acid sequence configured to hybridize to a nucleic acid molecule encoding one or more genes selected from an MTAP gene, a CDKN2A gene, a CDKN2B gene, an FGFR3 gene, a PTEN gene, an RB1 gene, an EGFR gene, a SMARCA4 gene, a KRAS gene, a TP53 gene, an NF2 gene, a VHL gene, or a PBRM1 gene. In some embodiments, the probe comprises a nucleic acid sequence configured to hybridize to a fragment or portion of a nucleic acid molecule encoding one or more genes selected from an MTAP gene, a CDKN2A gene, a CDKN2B gene, an FGFR3 gene, a PTEN gene, an RB1 gene, an EGFR gene, a SMARCA4 gene, a KRAS gene, a TP53 gene, an NF2 gene, a VHL gene, or a PBRM1 gene. In some embodiments, the one or more mutations in the KRAS gene result in a G12C amino acid substitution in a KRAS polypeptide encoded by the KRAS gene. In some embodiments, a probe provided herein comprises a nucleic acid sequence configured to hybridize to a target nucleic acid molecule, e.g., a target nucleic acid molecule comprising nucleotide sequences of one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the probe comprises a nucleic acid sequence configured to hybridize to a nucleic acid molecule encoding one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the probe comprises a nucleic acid sequence configured to hybridize to a fragment or portion of a nucleic acid molecule encoding one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides. In some embodiments, the probe comprises a nucleic acid sequence configured to hybridize to a nucleic acid molecule encoding an alteration described herein, e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703, e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides.

In some embodiments, the gene alteration is a gene fusion or a rearrangement. In some embodiments, the probe comprises a nucleotide sequence configured to hybridize to a breakpoint of a gene fusion or rearrangement, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint. In some embodiments, the probe is configured to hybridize to a nucleotide sequence in an intron or an exon of a first gene of a gene fusion or rearrangement, and/or to a nucleotide sequence in an intron or an exon of a second gene of a gene fusion or rearrangement, and/or to a nucleotide sequence in a breakpoint of a gene fusion or rearrangement (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe is configured to hybridize to a breakpoint joining an intron or a exon of first gene and an intron or an exon of a second gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). For example, a probe provided herein may be used for detecting one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) in a sample, e.g., a sample obtained from an individual. In some embodiments, the probe may be used for identifying cells or tissues that express one or more genes comprising one or more alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), e.g., by measuring levels of a nucleic acid molecule (e.g., a transcribed nucleic acid molecule, such as an RNA or an mRNA) comprising the one or more alterations. In some embodiments, the probe may be used for detecting levels of one or more genes comprising one or more alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), e.g., mRNA levels, in a sample of cells from an individual.

In some embodiments, a probe provided herein specifically hybridizes to a nucleic acid comprising a rearrangement (e.g., a deletion, inversion, insertion, duplication, or other rearrangement) resulting in one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703).

In some embodiments, a probe of the disclosure distinguishes a nucleic acid, e.g., a genomic or transcribed nucleic acid, e.g., a cDNA or RNA, having one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), from a reference nucleotide sequence, e.g., a nucleotide sequence not having the one or more alterations.

Also provided herein are isolated pairs of allele-specific probes, wherein, for example, the first probe of the pair specifically hybridizes to a nucleic acid molecule comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), and the second probe of the pair specifically hybridizes to a corresponding wild type sequence (e.g., a wild type AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 gene nucleotide sequence). Probe pairs can be designed and produced for any of the gene alterations described herein and are useful in detecting a somatic mutation in a sample. In some embodiments, a first probe of a pair specifically hybridizes to a mutation (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 10′ nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703).

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin (BIO)-11-dUTP, Digoxygenin (DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules are used, or further processing is performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a nucleotide sequence encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), and a sequence on either side of the one or more gene alterations (e.g., any of 1, 2, 3, 4, 5, 6, 7, 8, 9, or 10 nucleotides on either side of the one or more gene alterations, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the one or more gene alterations).

Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment or portion thereof. In some embodiments, an oligonucleotide, e.g., a primer, provided herein is configured to hybridize to a nucleic acid molecule encoding one or more genes selected from an MTAP gene, CDKN2A gene, CDKN2B gene, FGFR3 gene, PTEN gene, an RB1 gene, an EGFR gene, a SMARCA4 gene, a KRAS gene, a TP53 gene, an NF2 gene, a VHL gene, or a PBRM1 gene. In some embodiments, an oligonucleotide, e.g., a primer, provided herein is configured to hybridize to a fragment of a nucleic acid molecule encoding one or more genes selected from an MTAP gene, CDKN2A gene, CDKN2B gene, FGFR3 gene, PTEN gene, an RB1 gene, an EGFR gene, a SMARCA4 gene, a KRAS gene, a TP53 gene, an NF2 gene, a VHL gene, or a PBRM1 gene. In some embodiments, the one or more mutations in the KRAS gene result in a G12C amino acid substitution in a KRAS polypeptide encoded by the KRAS gene. In some embodiments, an oligonucleotide, e.g., a primer, provided herein is configured to hybridize to a nucleic acid molecule encoding one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, an oligonucleotide, e.g., a primer, provided herein is configured to hybridize to a fragment of a nucleic acid molecule encoding one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the oligonucleotide comprises a nucleic acid sequence configured to hybridize to a nucleotide sequence encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), e.g., plus or minus any of between about 10 and about 12, about 12 and about 15, about 15 and about 17, about 17 and about 20, about 20 and about 25, or about 25 and about 30, or more nucleotides.

In some embodiments, the gene alteration is a gene fusion or a rearrangement. In some embodiments, the oligonucleotide comprises a nucleotide sequence configured to hybridize to a breakpoint of a gene fusion or rearrangement, and may be further configured to hybridize to between about 10 and about 12, about 12 and about 15, about 15 and about 17, about 17 and about 20, about 20 and about 25, or about 25 and about 30, or more nucleotides flanking either side of the breakpoint. In some embodiments, the oligonucleotide is configured to hybridize to a nucleotide sequence in an intron or an exon of a first gene of a gene fusion or rearrangement, and/or to a nucleotide sequence in an intron or an exon of a second gene of a gene fusion or rearrangement, and/or to a nucleotide sequence in a breakpoint of a gene fusion or rearrangement (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide is configured to hybridize to a breakpoint joining an intron or a exon of first gene and an intron or an exon of a second gene (e.g., plus or minus any of between about 10 and about 12, about 12 and about 15, about 15 and about 17, about 17 and about 20, about 20 and about 25, or about 25 and about 30, or more nucleotides).

In some embodiments, the oligonucleotide comprises a nucleotide sequence corresponding to a nucleic acid molecule encoding one or more genes, e.g., an MTAP gene, CDKN2A gene, CDKN2B gene, FGFR3 gene, PTEN gene, an RB1 gene, an EGFR gene, a SMARCA4 gene, a KRAS gene, a TP53 gene, an NF2 gene, a VHL gene, or a PBRM1 gene. In some embodiments, the one or more mutations in the KRAS gene result in a G12C amino acid substitution in a KRAS polypeptide encoded by the KRAS gene. In some embodiments, the oligonucleotide comprises a nucleotide sequence corresponding to a nucleic acid molecule encoding one or more genes, e.g., AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the oligonucleotide comprises a nucleotide sequence corresponding to a nucleic acid molecule encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In some embodiments, the oligonucleotide comprises a nucleotide sequence corresponding to a fragment or a portion of a nucleic acid molecule encoding one or more genes, e.g., an MTAP gene, CDKN2A gene, CDKN2B gene, FGFR3 gene, PTEN gene, an RB1 gene, an EGFR gene, a SMARCA4 gene, a KRAS gene, a TP53 gene, an NF2 gene, a VHL gene, or a PBRM1 gene. In some embodiments, the one or more mutations in the KRAS gene result in a G12C amino acid substitution in a KRAS polypeptide encoded by the KRAS gene. In some embodiments, the oligonucleotide comprises a nucleotide sequence corresponding to a fragment or a portion of a nucleic acid molecule encoding one or more genes, e.g., AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the oligonucleotide comprises a nucleotide sequence corresponding to a fragment or a portion of a nucleic acid molecule encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In some embodiments, the fragment or portion comprises between about 10 and about 30 nucleotides, between about 12 and about 20 nucleotides, or between about 12 and about 17 nucleotides.

In some embodiments, the oligonucleotide comprises a nucleotide sequence complementary to a nucleic acid molecule encoding one or more genes, e.g., an MTAP gene, CDKN2A gene, CDKN2B gene, FGFR3 gene, PTEN gene, an RB1 gene, an EGFR gene, a SMARCA4 gene, a KRAS gene, a TP53 gene, an NF2 gene, a VHL gene, or a PBRM1 gene. In some embodiments, the one or more mutations in the KRAS gene result in a G12C amino acid substitution in a KRAS polypeptide encoded by the KRAS gene. In some embodiments, the oligonucleotide comprises a nucleotide sequence complementary to a nucleic acid molecule encoding one or more genes, e.g., AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the oligonucleotide comprises a nucleotide sequence complementary to a nucleic acid molecule encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In some embodiments, the oligonucleotide comprises a nucleotide sequence complementary to a fragment or a portion of a nucleic acid molecule encoding one or more genes, e.g., an MTAP gene, CDKN2A gene, CDKN2B gene, FGFR3 gene, PTEN gene, RB1 gene, EGFR gene, SMARCA4 gene, KRAS gene, TP53 gene, NF2 gene, VHL gene, or PBRM1 gene. In some embodiments, the one or more mutations in the KRAS gene result in a G12C amino acid substitution in a KRAS polypeptide encoded by the KRAS gene. In some embodiments, the oligonucleotide comprises a nucleotide sequence complementary to a fragment or a portion of a nucleic acid molecule encoding one or more genes, e.g., AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703. In some embodiments, the oligonucleotide comprises a nucleotide sequence complementary to a fragment or a portion of a nucleic acid molecule encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In some embodiments, the fragment or portion comprises between about 10 and about 30 nucleotides, between about 12 and about 20 nucleotides, or between about 12 and about 17 nucleotides. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment thereof, e.g., using PCR. In some embodiments, the oligonucleotide may be used to sequence a nucleic acid molecule comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment thereof. In some embodiments, the oligonucleotide may be used to amplify a nucleic acid molecule comprising a breakpoint of a fusion or rearrangement provided herein, e.g., using PCR. In some embodiments, the oligonucleotide may be used to sequence a nucleic acid molecule comprising a breakpoint a fusion or rearrangement provided herein.

In some embodiments, pairs of oligonucleotides, e.g., pairs of primers, are provided herein, which are configured to hybridize to a nucleic acid molecule comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment thereof. In some embodiments, a pair of oligonucleotides of the disclosure may be used for directing amplification of a nucleic acid molecule or fragment thereof comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), e.g., using a PCR reaction. In some embodiments, pairs of oligonucleotides, e.g., pairs of primers, are provided herein, which are configured to hybridize to a nucleic acid molecule encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), e.g., for use in directing amplification, e.g., using a PCR reaction. In some embodiments, pairs of oligonucleotides, e.g., pairs of primers, are provided herein, which are configured to hybridize to a nucleic acid molecule comprising a breakpoint of a fusion or rearrangement provided herein, e.g., for use in directing amplification of a fusion nucleic acid molecule, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a nucleic acid molecule comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), from a reference nucleotide sequence, e.g., a nucleotide sequence not having the one or more alterations.

In one aspect, provided herein is a primer or primer set for amplifying a nucleic acid molecule comprising a cytogenetic abnormality such as a chromosomal inversion, deletion, translocation, duplication, or other rearrangement resulting in one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In another aspect, provided herein is a primer or primer set for amplifying a nucleic acid molecule comprising a chromosomal inversion, insertion, deletion, translocation, duplication or other rearrangement resulting in one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In certain aspects, provided herein are allele-specific oligonucleotides, e.g., primers, wherein a first oligonucleotide of a pair specifically hybridizes to a mutation (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), and a second oligonucleotide of the pair specifically hybridizes to a sequence downstream of the mutation.

In some embodiments, the oligonucleotide, e.g., the primer, hybridizes to the one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), and a sequence on either side of the one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), e.g., any of 1, 2, 3, 4, 5, 6, 7, 8, 9, or 10 nucleotides on either side of the one or more gene alterations, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the one or more gene alterations).

Nucleic Acid Samples

A variety of materials (such as tissues) can be the source of the nucleic acid samples used in the methods provided herein. For example, the source of the sample can be solid tissue as from a fresh, frozen and/or preserved organ, tissue sample, biopsy, resection, smear, or aspirate; blood or any blood constituents; bodily fluids such as cerebrospinal fluid, amniotic fluid, urine, saliva, sputum, peritoneal fluid or interstitial fluid; or cells from any time in gestation or development of an individual. In some embodiments, the source of the sample is blood or blood constituents. In some embodiments, the source of the sample is a tumor sample, e.g., a UCB, a NSCLC, a srcRCC or a ccRCC tumor sample. In some embodiments, the sample is or comprises biological tissue or fluid. In some embodiments, the sample can contain compounds that are not naturally intermixed with the tissue in nature, such as preservatives, anticoagulants, buffers, fixatives, nutrients, antibiotics or the like. In some embodiments, the sample comprises genomic or subgenomic DNA fragments, or RNA, such as mRNA isolated from a sample, e.g., a tumor sample (e.g., from a UCB, a NSCLC, a srcRCC or a ccRCC), a normal adjacent tissue (NAT) sample, a tissue sample, or a blood sample obtained from an individual. In some embodiments, the sample comprises cDNA derived from an mRNA sample or from a sample comprising mRNA. In some embodiments, the tissue is preserved as a frozen sample or as a formaldehyde- or paraformaldehyde-fixed paraffin-embedded (FFPE) tissue preparation. For example, the sample can be embedded in a matrix, e.g., an FFPE block or a frozen sample.

In some embodiments, the sample comprises cell-free DNA (cfDNA). In some embodiments, the sample comprises cell-free RNA (cfRNA). In some embodiments, the sample comprises circulating nucleic acids. In some embodiments, the sample comprises circulating tumor DNA (ctDNA).

In some embodiments, a sample may be or comprise bone marrow; a bone marrow aspirate; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; other body fluids, secretions, and/or excretions; and/or cells therefrom. In some embodiments, a biological sample is or comprises cells obtained from an individual.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example nucleic acids or proteins extracted from a sample or obtained by subjecting a primary sample to techniques such as amplification or reverse transcription of mRNA, or isolation and/or purification of certain components.

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein, e.g., a UCB, NSCLC, srcRCC, or ccRCC. In some embodiments, the sample is acquired from a solid tumor, a soft tissue tumor or a metastatic lesion. In other embodiments, the sample includes tissue or cells from a surgical margin. In another embodiment, the sample includes one or more circulating tumor cells (CTCs) (e.g., a CTC acquired from a blood sample). In one embodiment, the sample is a cell not associated with a tumor, e.g., a non-tumor cell or a peripheral blood lymphocyte.

In some embodiments, the sample comprises tumor nucleic acids, such as nucleic acids from a tumor or a cancer sample, e.g., genomic DNA, RNA, or cDNA derived from RNA, from a tumor or cancer sample. In certain embodiments, a tumor nucleic acid sample is purified or isolated (e.g., it is removed from its natural state).

In some embodiments, the sample is a control nucleic acid sample or a reference nucleic acid sample, e.g., genomic DNA, RNA, or cDNA derived from RNA, not containing a mutation or gene fusion described herein. In certain embodiments, the reference or control nucleic acid sample comprises a wild type or a non-mutated sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

Detection of Gene Alterations in Polypeptides

Also provided herein are methods of detecting one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) in an encoded polypeptide, or a fragment thereof. A polypeptide encoded by a gene comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, a polypeptide encoded by a gene comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment thereof, can be distinguished from a reference polypeptide, e.g., a corresponding non-mutant or wild type protein or polypeptide, with an antibody or antibody fragment that reacts differentially with a mutant protein or polypeptide (e.g., a polypeptide encoded by a gene comprising one or more gene alterations of the disclosure) as compared to a reference protein or polypeptide. In some embodiments, a polypeptide encoded by a gene comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment thereof, canbe distinguished from a reference polypeptide, e.g., a corresponding non-mutant or wild type protein or polypeptide, by reaction with a detection reagent, e.g., a substrate, e.g., a substrate for catalytic activity, e.g., phosphorylation (e.g., kinase activity), dephosphorylation (e.g., phosphatase activity), or another suitable catalytic activity.

In some aspects, methods of detection of a polypeptide encoded by a gene comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment thereof, are provided, comprising contacting a sample, e.g., a sample described herein, comprising a polypeptide encoded by a gene comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), with a detection reagent provided herein (e.g., an antibody of the disclosure), and determining if the polypeptide encoded by a gene comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) is present.

Protein Samples

A variety of materials (such as tissues) can be the source of protein samples used in the methods provided herein. For example, the source of the sample can be a solid tissue, e.g., from a fresh, frozen and/or preserved organ, tissue sample, biopsy (e.g., a tumor biopsy), resection, smear, or aspirate; blood or any blood constituents; bodily fluids such as cerebrospinal fluid, amniotic fluid, urine, saliva, sputum, peritoneal fluid or interstitial fluid; or cells such as tumor cells. In some embodiments, the source of the sample is blood or blood constituents. In some embodiments, the source of the sample is a tumor sample. In some embodiments, the sample is or comprises biological tissue or fluid. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In some embodiments, a sample for use according to the methods of detection of one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703) is a sample of proteins isolated or obtained from a solid tissue, e.g., from a fresh, frozen and/or preserved organ, tissue sample, biopsy (e.g., a tumor biopsy, e.g., from a UCB, a NSCLC, a srcRCC or a ccRCC), resection, smear, or aspirate; from blood or any blood constituents; from bodily fluids such as cerebrospinal fluid, amniotic fluid, urine, saliva, sputum, peritoneal fluid or interstitial fluid; or from cells such as tumor cells. In some embodiments, the sample is a sample of proteins isolated or obtained from a preserved sample, such as a frozen sample or a formaldehyde- or paraformaldehyde-fixed paraffin-embedded (FFPE) tissue preparation. In some embodiments, the sample is a sample of proteins isolated or obtained from circulating tumor cells (CTCs), e.g., from a UCB, a NSCLC, a srcRCC or a ccRCC. In some embodiments, the sample can contain compounds that are not naturally intermixed with the tissue in nature, such as preservatives, anticoagulants, buffers, fixatives, nutrients, antibiotics or the like.

In some embodiments, a sample may be or comprise bone marrow; a bone marrow aspirate; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; other body fluids, secretions, and/or excretions; and/or cells therefrom. In some embodiments, a biological sample is or comprises cells obtained from an individual.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, proteins extracted from a sample or obtained by subjecting a primary sample to techniques such as isolation and/or purification of certain components.

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein, e.g., a UCB, NSCLC, srcRCC, or ccRCC. In some embodiments, the sample is acquired from a solid tumor, a soft tissue tumor or a metastatic lesion. In other embodiments, the sample includes tissue or cells from a surgical margin. In another embodiment, the sample includes one or more circulating tumor cells (CTCs) (e.g., a CTC acquired from a blood sample). In one embodiment, the sample is a cell not associated with a tumor, e.g., a non-tumor cell or a peripheral blood lymphocyte.

In some embodiments, the sample comprises tumor proteins or polypeptides, such as proteins or polypeptides from a tumor or a cancer sample, e.g., from a UCB, a NSCLC, a srcRCC or a ccRCC. In certain embodiments, the proteins are purified or isolated (e.g., removed from their natural state).

In some embodiments, the sample is a control sample or a reference sample, e.g., not containing a polypeptide encoded by a gene comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In certain embodiments, the reference sample is purified or isolated (e.g., it is removed from its natural state). In other embodiments, the reference sample is from a non-tumor sample, e.g., a blood control, a normal adjacent tumor (NAT), or any other non-cancerous sample from the same or a different subject.

In some embodiments of any of the methods provided herein, the presence or absence of a deletion of an MTAP gene, or of a portion thereof, is assessed using an immunohistochemistry assay, e.g., a Ventana IHC MTAP assay, e.g., a Ventana MS assay.

### Tumor Mutational Burden

In some embodiments, the methods provided herein comprise acquiring knowledge that a UCB has a tumor mutational burden of at least about 10 mut/Mb or at least about 20 mut/Mb. In some embodiments, acquiring knowledge that a UCB has a tumor mutational burden of at least about 10 mut/Mb or at least about 20 mut/Mb comprises measuring the level of tumor mutational burden in a sample, e.g., in a sample from a UCB tumor, obtained from an individual. In some embodiments, the methods provided herein comprise detecting a tumor mutational burden of at least about 10 mut/Mb or at least about 20 mut/Mb in a sample from an individual having a UCB. In some embodiments, the methods comprise administering an effective amount of an immunotherapy responsive to knowledge that the UCB has a tumor mutational burden of at least about 10 mut/Mb or at least about 20 mut/Mb. In some embodiments, the methods comprise providing a report to a party.

In some embodiments, the methods provided herein comprise acquiring knowledge that a srcRCC or ccRCC has a tumor mutational burden of at least about 10 mut/Mb or at least about 20 mut/Mb. In some embodiments, acquiring knowledge that a srcRCC or ccRCC has a tumor mutational burden of at least about 10 mut/Mb or at least about 20 mut/Mb comprises measuring the level of tumor mutational burden in a sample, e.g., in a sample from a srcRCC or ccRCC tumor, obtained from an individual. In some embodiments, the methods provided herein comprise detecting a tumor mutational burden of at least about 10 mut/Mb or at least about 20 mut/Mb in a sample from an individual having a srcRCC or ccRCC. In some embodiments, the methods comprise administering an effective amount of an immunotherapy responsive to knowledge that the srcRCC or ccRCC has a tumor mutational burden of at least about 10 mut/Mb or at least about 20 mut/Mb. In some embodiments, the methods comprise providing a report to a party.

In some embodiments, the methods provided herein comprise acquiring knowledge that a NSCLC has a tumor mutational burden of at least about 10 mut/Mb or at least about 20 mut/Mb. In some embodiments, acquiring knowledge that a NSCLC has a tumor mutational burden of at least about 10 mut/Mb or at least about 20 mut/Mb comprises measuring the level of tumor mutational burden in a sample, e.g., in a sample from a NSCLC tumor, obtained from an individual. In some embodiments, the methods provided herein comprise detecting a tumor mutational burden of at least about 10 mut/Mb or at least about 20 mut/Mb in a sample from an individual having a NSCLC. In some embodiments, the methods comprise administering an effective amount of an immunotherapy responsive to knowledge that the NSCLC has a tumor mutational burden of at least about 10 mut/Mb or at least about 20 mut/Mb. In some embodiments, the methods comprise providing a report to a party.

In some embodiments, tumor mutational burden is assessed in sample from an individual, such as sample described herein. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, genomic DNA, circulating tumor DNA, cell-free DNA, or cell-free RNA.

In some embodiments, tumor mutational burden is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

In some embodiments, tumor mutational burden is measured according to the methods provided in WO2017151524A1, which is hereby incorporated by reference in its entirety.

In some embodiments, tumor mutational burden is measured in the sample by whole exome sequencing. In some embodiments, tumor mutational burden is measured in the sample using next-generation sequencing. In some embodiments, tumor mutational burden is measured in the sample using whole genome sequencing. In some embodiments, tumor mutational burden is measured in the sample by gene-targeted sequencing. In some embodiments, tumor mutational burden is measured on between about 0.8 Mb and about 1.1 Mb of sequenced DNA. In some embodiments, tumor mutational burden is measured on any of about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, or about 1.1 Mb of sequenced DNA. In some embodiments, tumor mutational burden is measured on about 0.8 Mb of sequenced DNA.

In some embodiments, the UCB, NSCLC, srcRCC or ccRCC has a high tumor mutational burden, e.g., of at least about 10 mut/Mb. In some embodiments, the UCB, NSCLC, srcRCC or ccRCC has a tumor mutational burden of at least about 10 mut/Mb. In some embodiments, the UCB, NSCLC, srcRCC or ccRCC has a tumor mutational burden of at least about 20 mut/Mb. In some embodiments, the UCB, NSCLC, srcRCC or ccRCC has a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the UCB, NSCLC, srcRCC or ccRCC has a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb.

In some embodiments, measuring tumor mutational burden comprises assessing mutations in a sample derived from a cancer in an individual, e.g., a UCB, a NSCLC, a srcRCC or a ccRCC described herein. In some embodiments, measuring tumor mutational burden comprises assessing mutations in a sample derived from a cancer in an individual, e.g., a UCB, a NSCLC, a srcRCC or a ccRCC described herein, and in a matched normal sample, e.g., a sample from the individual derived from a tissue or other source that is free of the cancer.

PD-L1 Expression

In some embodiments, the methods provided herein comprise acquiring knowledge that a UCB is PD-L1-positive in a sample from an individual. In some embodiments, acquiring knowledge that the UCB is PD-L1-positive comprises measuring the level of PD-L1 expression in a sample, e.g., in a sample from a UCB tumor, obtained from an individual. In some embodiments, the methods provided herein comprise detecting a PD-L1-positive UCB in a sample from an individual. In some embodiments, detecting a PD-L1-positive UCB in a sample from an individual comprises measuring the level of PD-L1 expression in a sample, e.g., in a sample from a UCB tumor obtained from the individual. In some embodiments, the methods comprise administering an effective amount of an immunotherapy responsive to knowledge that the UCB is PD-L1-positive. In some embodiments, the methods comprise providing a report to a party.

In some embodiments, the methods provided herein comprise acquiring knowledge that a NSCLC is PD-L1-positive in a sample from an individual. In some embodiments, acquiring knowledge that the NSCLC is PD-L1-positive comprises measuring the level of PD-L1 expression in a sample, e.g., in a sample from a NSCLC tumor, obtained from an individual. In some embodiments, the methods provided herein comprise detecting a PD-L1-positive NSCLC in a sample from an individual. In some embodiments, detecting a PD-L1-positive NSCLC in a sample from an individual comprises measuring the level of PD-L1 expression in a sample, e.g., in a sample from a NSCLC tumor obtained from the individual. In some embodiments, the methods comprise administering an effective amount of an immunotherapy responsive to knowledge that the NSCLC is PD-L1-positive. In some embodiments, the methods comprise providing a report to a party.

In some embodiments, the methods provided herein comprise acquiring knowledge that a srcRCC or ccRCC is PD-L1-positive in a sample from an individual. In some embodiments, acquiring knowledge that the srcRCC or ccRCC is PD-L1-positive comprises measuring the level of PD-L1 expression in a sample, e.g., in a sample from a srcRCC or ccRCC tumor, obtained from an individual. In some embodiments, the methods provided herein comprise detecting a PD-L1-positive srcRCC or ccRCC in a sample from an individual. In some embodiments, detecting a PD-L1-positive srcRCC or ccRCC in a sample from an individual comprises measuring the level of PD-L1 expression in a sample, e.g., in a sample from a srcRCC or ccRCC tumor obtained from the individual. In some embodiments, the methods comprise administering an effective amount of an immunotherapy responsive to knowledge that the srcRCC or ccRCC is PD-L1-positive. In some embodiments, the methods comprise providing a report to a party.

Any suitable method for measuring PD-L1 expression in a sample from an individual may be used. For example, the level of PD-L1 expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used.

In some embodiments, PD-L1 expression in a sample from an individual is measured based on the level of PD-L1 mRNA in the sample. Any suitable method for measuring mRNA expression in a sample from an individual may be used. For example, the level of PD-L1 mRNA expression may be measured using in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”).

In some embodiments, PD-L1 expression in a sample from an individual is measured based on the level of PD-L1 protein in the sample. Any suitable method for measuring protein expression in a sample from an individual may be used. For example, the level of PD-L1 protein expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), proteomics (e.g., mass spectrometry), quantitative blood based assays (as for example serum ELISA), biochemical enzymatic activity assays, or multiplexed immunoassays such as those available from Rules Based Medicine or Meso Scale Discovery (“MSD”).

In some embodiments, the level of PD-L1 protein expression is measured using an immunohistochemistry assay. In some embodiments, the level of PD-L1 protein expression is measured using a VENTANA PD-L1 assay (SP142). In some embodiments, the level of PD-L1 protein expression is determined based on PD-L1 expression in tumor infiltrating immune cells (ICs) and/or tumor cells (TCs). Additional information about the VENTANA SP142 assay may be found in the website: www[dot]accessdata[dot]fda[dot]gov/cdrh_docs/pdf16/P160002c.pdf. In some embodiments, the level of PD-L1 protein expression is measured using a PD-L1 IHC assay such as a DAKO 22C3 assay. In some embodiments, the level of PD-L1 protein expression is determined based on a combined positive score (CPS), which is the number of PD-L1 staining cells (e.g., tumor cells, lymphocytes, macrophages or histiocytes) divided by the total number of viable tumor cells, and multiplied by 100. Additional information about the DAKO 22C3 assay may be found in the website: www[dot]agilent[dot]com/cs/library/usermanuals/public/29219_pd-11-ihc-22C3-pharmdx-gastric-interpretation-manual_us.pdf. In some embodiments, a sample from an individual is determined to be positive for PD-L1 if it has a combined positive score (CPS) of at least about 10. In some embodiments, a sample from an individual has a combined positive score (CPS) of at least about 10. In some embodiments, a sample from an individual has a combined positive score (CPS) of between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, or about 100.

In some embodiments, a UCB, NSCLC, srcRCC or ccRCC provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) express PD-L1 protein and/or PD-L1 mRNA (e.g., are positive for PD-L1 protein and/or PD-L1 mRNA). In some embodiments, a UCB, NSCLC, srcRCC or ccRCC provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) express PD-L1 protein and/or PD-L1 mRNA (e.g., are positive for PD-L1 protein and/or PD-L1 mRNA). In some embodiments, a UCB, NSCLC, srcRCC or ccRCC provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor cells express PD-L1 protein and/or PD-L1 mRNA (e.g., are positive for PD-L1 protein and/or PD-L1 mRNA).

In some embodiments, a UCB, NSCLC, srcRCC or ccRCC provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in a sample from an individual express PD-L1 protein and/or PD-L1 mRNA (e.g., are positive for PD-L1 protein and/or PD-L1 mRNA). In some embodiments, a UCB, NSCLC, srcRCC or ccRCC provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) in a sample from an individual express PD-L1 protein and/or PD-L1 mRNA (e.g., are positive for PD-L1 protein and/or PD-L1 mRNA). In some embodiments, a UCB, NSCLC, srcRCC or ccRCC provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor cells in a sample from an individual express PD-L1 protein and/or PD-L1 mRNA (e.g., are positive for PD-L1 protein and/or PD-L1 mRNA).

In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in the sample express PD-L1 protein and/or PD-L1 mRNA (e.g., are positive for PD-L1 protein and/or PD-L1 mRNA). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) in the sample express PD-L1 protein and/or PD-L1 mRNA (e.g., are positive for PD-L1 protein and/or PD-L1 mRNA). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor cells in the sample express PD-L1 protein and/or PD-L1 mRNA (e.g., are positive for PD-L1 protein and/or PD-L1 mRNA).

In some embodiments, the level of PD-L1 protein and/or PD-L1 mRNA is assessed in sample from an individual, such as a sample described herein. In some embodiments, the sample from the individual comprises fluid, cells, or tissue. In some embodiments, the sample from the individual comprises a tumor biopsy or a circulating tumor cell. In some embodiments, the sample is obtained or derived from a UCB. In some embodiments, the sample is obtained or derived from a NSCLC. In some embodiments, the sample is obtained or derived from a srcRCC or ccRCC.

In some embodiments of any of the methods provided herein, a sample from an individual, e.g., an individual having a UCB, NSCLC, srcRCC or ccRCC, is determined to be PD-L1-negative if 0% of tumor cells in the sample express PD-L1. In some embodiments of any of the methods provided herein, a sample from an individual, e.g., an individual having a UCB, NSCLC, srcRCC or ccRCC, is determined to be PD-L1 positive if at least about 1% of tumor cells in the sample express PD-L1. In some embodiments of any of the methods provided herein, a sample from an individual, e.g., an individual having a UCB, NSCLC, srcRCC or ccRCC, is determined to be PD-L1 low positive if between about 1% and about 49% of tumor cells in the sample express PD-L1. In some embodiments of any of the methods provided herein, a sample from an individual, e.g., an individual having a UCB, NSCLC, srcRCC or ccRCC, is determined to be PD-L1 high positive if at least about 50% or more of tumor cells in the sample express PD-L1. In some embodiments of any of the methods provided herein, a sample from an individual, e.g., an individual having a UCB, NSCLC, srcRCC or ccRCC, is determined to be PD-L1 positive if the sample is PD-L1 low positive or PD-L1 high positive.

In some embodiments, the level of PD-L1 protein expression is determined based on PD-L1 tumor cell expression using an immunohistochemistry assay, such as a DAKO 22C3 assay. In some embodiments, the level of PD-L1 protein expression is assessed based on a tumor proportion score (TPS). The TPS is the percentage of viable tumor cells showing partial or complete PD-L1 membrane staining (e.g., at a ≥1+ intensity on a 0, 1+, 2+, and 3 scale) relative to all viable tumor cells present in the sample. In some embodiments, the TPS is calculated as: the number of PD-L1-positive tumor cells/Total number of PD-L1-positive tumor cells+Total number of PD-L1-negative tumor cells. As used herein, a PD-L1 low positive status is defined as a TPS of between 1% and 49%, and PD-L1 high positive status is defined as a TPS of 50% or greater. Additional information about the DAKO 22C3 assay and the TPS score may be found, e.g., in the website: www.agilent.com/cs/library/usennanuals/public/29158_pd-11-ihc-22C3-pharmdx-nsclc-interpretation-manual.pdf.

In some embodiments, a cancer of the disclosure is determined to be PD-L1 positive if it has PD-L1 low positive status or a PD-L1 high positive status. In some embodiments, a cancer of the disclosure is PD-L1 positive (e.g., the cancer is determined have a TPS of any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%, in a sample obtained from an individual having the cancer). In some embodiments, a cancer of the disclosure is PD-L1 low positive (e.g., the cancer is determined have a TPS of any of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, or about 49%, in a sample obtained from an individual having the cancer). In some embodiments, a cancer of the disclosure is PD-L1 high positive (e.g., the cancer is determined have a TPS of any of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, in a sample obtained from an individual having the cancer).

Systems, Software, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform a method according to any of the embodiments described herein.

In some aspects, provided herein are systems comprising one or more processors and a non-transitory computer-readable storage media.

In some embodiments, a system of the disclosure comprises one or more processors; and a non-transitory computer readable storage medium comprising one or more programs executable by the one or more processors for performing a method.

In some embodiments, the method comprises one or more of: (a) sequencing one or more nucleic acids, thereby producing a plurality of sequencing reads, wherein the one or more nucleic acids are derived from a sample obtained from an individual having a UCB; (b) analyzing the plurality of sequencing reads for the presence of a deletion of an MTAP gene, or of a portion thereof, and one or more mutations in an FGFR3 gene or in a PTEN gene; and (c) detecting, based on the analyzing step, a deletion of an MTAP gene, or of a portion thereof, and one or more mutations in an FGFR3 gene or in a PTEN gene in the sample.

In some embodiments, the method comprises one or more of: (a) sequencing one or more nucleic acids, thereby producing a plurality of sequencing reads, wherein the one or more nucleic acids are derived from a sample obtained from an individual having a UCB; (b) analyzing the plurality of sequencing reads for the presence of a TMB of at least about 10 mut/Mb or at least about 20 mut/Mb, and/or one or more mutations in an RB1 gene; and (c) detecting, based on the analyzing step, a TMB of at least about 10 mut/Mb or at least about 20 mut/Mb, and/or one or more mutations in an RB1 gene in the sample.

In some embodiments, the method comprises one or more of: (a) sequencing one or more nucleic acids, thereby producing a plurality of sequencing reads, wherein the one or more nucleic acids are derived from a sample obtained from an individual having a UCB; (b) analyzing the plurality of sequencing reads for the presence of one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703; and (c) detecting, based on the analyzing step, one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the sample.

In some embodiments, a system of the disclosure comprises a memory configured to store one or more program instructions; and one or more processors configured to execute the one or more program instructions.

In some embodiments, the one or more program instructions when executed by the one or more processors are configured to: (a) obtain a plurality of sequence reads of one or more nucleic acids, wherein the one or more nucleic acids are derived from a sample obtained from an individual having a urothelial bladder cancer (UCB); (b) analyze the plurality of sequence reads for the presence of a deletion of an methylthioadenosine phosphorylase (MTAP) gene, or of a portion thereof, and one or more mutations in a fibroblast growth factor receptor 3 (FGFR3) gene or in a phosphatase and tensin homolog (PTEN) gene; and (c) detect, based on the analyzing, a deletion of an MTAP gene, or of a portion thereof, and one or more mutations in an FGFR3 gene or in a PTEN gene in the sample.

In some embodiments, the one or more program instructions when executed by the one or more processors are configured to: (a) obtain a plurality of sequence reads of one or more nucleic acids, wherein the one or more nucleic acids are derived from a sample obtained from an individual having a urothelial bladder cancer (UCB); (b) analyze the plurality of sequence reads for the presence of a tumor mutational burden (TMB) of at least about 10 mut/Mb or at least about 20 mut/Mb, and/or one or more mutations in a retinoblastoma (RB1) gene; and (c) detect, based on the analyzing, a TMB of at least about 10 mut/Mb or at least about 20 mut/Mb, and/or one or more mutations in an RB1 gene in the sample.

In some embodiments, the one or more program instructions when executed by the one or more processors are configured to: (a) obtain a plurality of sequence reads of one or more nucleic acids, wherein the one or more nucleic acids are derived from a sample obtained from an individual having a urothelial bladder cancer (UCB); (b) analyze the plurality of sequence reads for the presence of a deletion of an methylthioadenosine phosphorylase (MTAP) gene, or of a portion thereof, and one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703; and (c) detect, based on the analyzing, a deletion of an MTAP gene, or of a portion thereof, and one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the sample.

In some embodiments, the one or more program instructions when executed by the one or more processors are configured to: (a) obtain a plurality of sequence reads of one or more nucleic acids, wherein the one or more nucleic acids are derived from a sample obtained from an individual having a urothelial bladder cancer (UCB); (b) analyze the plurality of sequence reads for the presence of a tumor mutational burden (TMB) of at least about 10 mut/Mb or at least about 20 mut/Mb, and/or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703; and (c) detect, based on the analyzing, a TMB of at least about 10 mut/Mb or at least about 20 mut/Mb, and/or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the sample.

In some embodiments, the method comprises one or more of: (a) sequencing one or more nucleic acids, thereby producing a plurality of sequencing reads, wherein the one or more nucleic acids are derived from a sample obtained from an individual having a NSCLC; (b) analyzing the plurality of sequencing reads for the presence of a deletion of an MTAP gene, or of a portion thereof, and one or more mutations in a EGFR gene or in a SMARCA4 gene; and (c) detecting, based on the analyzing step, a deletion of an MTAP gene, or of a portion thereof, and one or more mutations in a EGFR gene or in a SMARCA4 gene in the sample.

In some embodiments, the method comprises one or more of: (a) sequencing one or more nucleic acids, thereby producing a plurality of sequencing reads, wherein the one or more nucleic acids are derived from a sample obtained from an individual having a NSCLC; (b) analyzing the plurality of sequencing reads for the presence of a TMB of at least about 10 mut/Mb or at least about 20 mut/Mb, and/or one or more mutations in an RB1 gene, in a TP53 gene or in a KRAS gene; and (c) detecting, based on the analyzing step, a TMB of at least about 10 mut/Mb or at least about 20 mut/Mb, and/or one or more mutations in an RB1 gene, in a TP53 gene or in a KRAS gene in the sample. In some embodiments, the one or more mutations in the KRAS gene result in a G12C amino acid substitution in a KRAS polypeptide encoded by the KRAS gene.

In some embodiments, the method comprises one or more of: (a) sequencing one or more nucleic acids, thereby producing a plurality of sequencing reads, wherein the one or more nucleic acids are derived from a sample obtained from an individual having a NSCLC; (b) analyzing the plurality of sequencing reads for the presence of one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703; and (c) detecting, based on the analyzing step, one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the sample.

In some embodiments, a system of the disclosure comprises a memory configured to store one or more program instructions; and one or more processors configured to execute the one or more program instructions.

In some embodiments, the one or more program instructions when executed by the one or more processors are configured to: (a) obtain a plurality of sequence reads of one or more nucleic acids, wherein the one or more nucleic acids are derived from a sample obtained from an individual having a non-small cell lung cancer (NSCLC); (b) analyze the plurality of sequence reads for the presence of a deletion of an MTAP gene, or of a portion thereof, and one or more mutations in an EGFR gene or in a SMARCA4 gene; and (c) detect, based on the analyzing, a deletion of an MTAP gene, or of a portion thereof, and one or more mutations in an EGFR gene or in a SMARCA4 gene in the sample.

In some embodiments, the one or more program instructions when executed by the one or more processors are configured to: (a) obtain a plurality of sequence reads of one or more nucleic acids, wherein the one or more nucleic acids are derived from a sample obtained from an individual having a non-small cell lung cancer (NSCLC); (b) analyze the plurality of sequence reads for the presence of a tumor mutational burden (TMB) of at least about 10 mut/Mb or at least about 20 mut/Mb, and/or one or more mutations in a retinoblastoma (RB1) gene, in a TP53 gene or in a KRAS gene; and (c) detect, based on the analyzing, a TMB of at least about 10 mut/Mb or at least about 20 mut/Mb, and/or one or more mutations in an RB1 gene, in a TP53 gene or in a KRAS gene in the sample. In some embodiments, the one or more mutations in the KRAS gene result in a G12C amino acid substitution in a KRAS polypeptide encoded by the KRAS gene.

In some embodiments, the one or more program instructions when executed by the one or more processors are configured to: (a) obtain a plurality of sequence reads of one or more nucleic acids, wherein the one or more nucleic acids are derived from a sample obtained from an individual having a non-small cell lung cancer (NSCLC); (b) analyze the plurality of sequence reads for the presence of a deletion of an methylthioadenosine phosphorylase (MTAP) gene, or of a portion thereof, and one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703; and (c) detect, based on the analyzing, a deletion of an MTAP gene, or of a portion thereof, and one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the sample.

In some embodiments, the one or more program instructions when executed by the one or more processors are configured to: (a) obtain a plurality of sequence reads of one or more nucleic acids, wherein the one or more nucleic acids are derived from a sample obtained from an individual having a non-small cell lung cancer (NSCLC); (b) analyze the plurality of sequence reads for the presence of a tumor mutational burden (TMB) of at least about 10 mut/Mb or at least about 20 mut/Mb, and/or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703; and (c) detect, based on the analyzing, a TMB of at least about 10 mut/Mb or at least about 20 mut/Mb, and/or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the sample.

In some embodiments, the method comprises one or more of: (a) sequencing one or more nucleic acids, thereby producing a plurality of sequencing reads, wherein the one or more nucleic acids are derived from a sample obtained from an individual having a srcRCC or a ccRCC; (b) analyzing the plurality of sequencing reads for the presence of a deletion of an MTAP gene, or of a portion thereof, and one or more mutations in an NF2 gene; and (c) detecting, based on the analyzing step, a deletion of an MTAP gene, or of a portion thereof, and one or more mutations in an NF2 gene in the sample.

In some embodiments, the method comprises one or more of: (a) sequencing one or more nucleic acids, thereby producing a plurality of sequencing reads, wherein the one or more nucleic acids are derived from a sample obtained from an individual having a ccRCC; (b) analyzing the plurality of sequencing reads for the presence of one or more mutations in a VHL gene or in a PBRM1 gene; and (c) detecting, based on the analyzing step, a one or more mutations in a VHL gene or in a PBRM1 gene in the sample.

In some embodiments, the method comprises one or more of: (a) sequencing one or more nucleic acids, thereby producing a plurality of sequencing reads, wherein the one or more nucleic acids are derived from a sample obtained from an individual having a srcRCC or a ccRCC; (b) analyzing the plurality of sequencing reads for the presence of one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703; and (c) detecting, based on the analyzing step, one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the sample.

In some embodiments, a system of the disclosure comprises a memory configured to store one or more program instructions; and one or more processors configured to execute the one or more program instructions.

In some embodiments, the one or more program instructions when executed by the one or more processors are configured to: (a) obtain a plurality of sequence reads of one or more nucleic acids, wherein the one or more nucleic acids are derived from a sample obtained from an individual having a sarcomatoid renal cell carcinoma (srcRCC) or a clear cell renal cell carcinoma (ccRCC); (b) analyze the plurality of sequence reads for the presence of a deletion of an methylthioadenosine phosphorylase (MTAP) gene, or of a portion thereof, and one or more mutations in an NF2 gene; and (c) detect, based on the analyzing, a deletion of an MTAP gene, or of a portion thereof, and one or more mutations in an NF2 gene in the sample.

In some embodiments, the one or more program instructions when executed by the one or more processors are configured to: (a) obtain a plurality of sequence reads of one or more nucleic acids, wherein the one or more nucleic acids are derived from a sample obtained from an individual having a ccRCC; (b) analyze the plurality of sequence reads for the presence of one or more mutations in Von Hippel-Lindau tumor suppressor (VHL) gene or in a protein polybromo-1 (PBRM1) gene; and (c) detect, based on the analyzing, one or more mutations in a VHL gene or in a PBRM1 gene in the sample.

In some embodiments, the one or more program instructions when executed by the one or more processors are configured to: (a) obtain a plurality of sequence reads of one or more nucleic acids, wherein the one or more nucleic acids are derived from a sample obtained from an individual having a sarcomatoid renal cell carcinoma (srcRCC) or a clear cell renal cell carcinoma (ccRCC); (b) analyze the plurality of sequence reads for the presence of a deletion of an methylthioadenosine phosphorylase (MTAP) gene, or of a portion thereof, and one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703; and (c) detect, based on the analyzing, a deletion of an MTAP gene, or of a portion thereof, and one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the sample.

In some embodiments, the one or more program instructions when executed by the one or more processors are configured to: (a) obtain a plurality of sequence reads of one or more nucleic acids, wherein the one or more nucleic acids are derived from a sample obtained from an individual having a clear cell renal cell carcinoma (ccRCC); (b) analyze the plurality of sequence reads for the presence of one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703; and (c) detect, based on the analyzing, one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the sample.

In some embodiments of the any of the systems provided herein, the one or more processors, and the non-transitory computer readable storage medium comprising one or more programs executable by the one or more processors are within a device. In some embodiments, the non-transitory computer-readable storage media comprise one or more programs for execution by one or more processors of the device, the one or more programs including instructions which, when executed by the one or more processors, cause the device to perform the method according to any of the embodiments described herein.

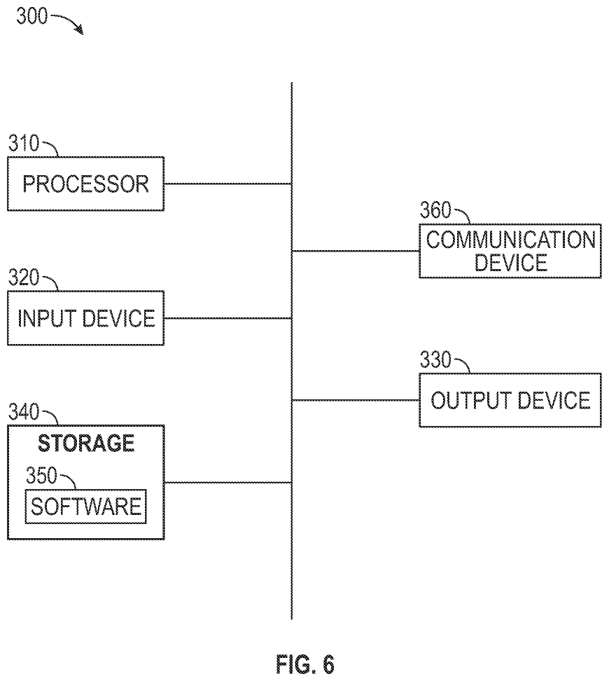

FIG. 6 illustrates an example of a computing device in accordance with one embodiment of a system of the present disclosure. As shown in FIG. 6, Device 300 can be a host computer connected to a network. Device 300 can be a client computer or a server. As shown in FIG. 6, Device 300 can be any suitable type of microprocessor-based device, such as a personal computer, workstation, server or handheld computing device (portable electronic device) such as a phone or tablet. In some embodiments, the device is a sequencer. In some embodiments, the device is incorporated into a sequencer. In some embodiments, the device is a part of a sequencer. The device can include, without limitation, one or more of processor 310, input device 320, output device 330, storage 340, and communication device 360. Input device 320 and output device 330 can generally correspond to those described above, and can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage (e.g., an electrical, magnetic or optical memory including a RAM, cache, hard drive, or removable storage disk). Communication device 360 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fi®, or any other wireless technology).

Software 350, which can be stored in storage 340 and executed by processor 310, can include, for example, the processes that embody the functionality of the present disclosure.

Software 350 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 340, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 300 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, TI or T3 lines, cable networks, DSL, or telephone lines.

Device 300 can implement any operating system suitable for operating on the network. Software 350 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

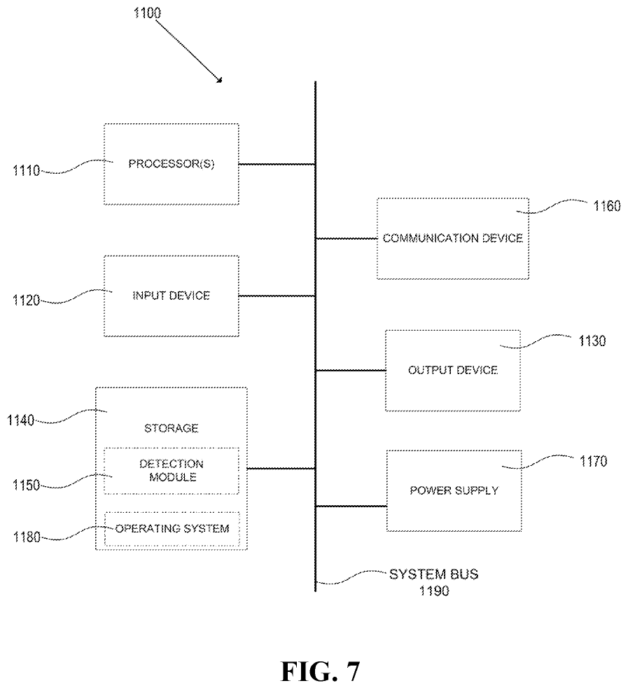

FIG. 7 illustrates an example of a computing device in accordance with one embodiment. Device 1100 can be a host computer connected to a network. Device 1100 can be a client computer or a server. As shown in FIG. 7, device 1100 can be any suitable type of microprocessor-based device, such as a personal computer, workstation, server or handheld computing device (portable electronic device) such as a phone or tablet. The device can include, for example, one or more of processor(s) 1110, input device 1120, output device 1130, storage 1140, communication device 1160, power supply 1170, operating system 1180, and system bus 1190. Input device 1120 and output device 1130 can generally correspond to those described herein, and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage (e.g., an electrical, magnetic or optical memory including a RAM (volatile and non-volatile), cache, hard drive, or removable storage disk). Communication device 1160 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fi®, or any other wireless technology). For example, in FIG. 7, the components are connected by System Bus 1190.

Detection module 1150, which can be stored as executable instructions in storage 1140 and executed by processor(s) 1110, can include, for example, the processes that embody the functionality of the present disclosure (e.g., as embodied in the devices as described herein).

Detection module 1150 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1140, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Detection module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

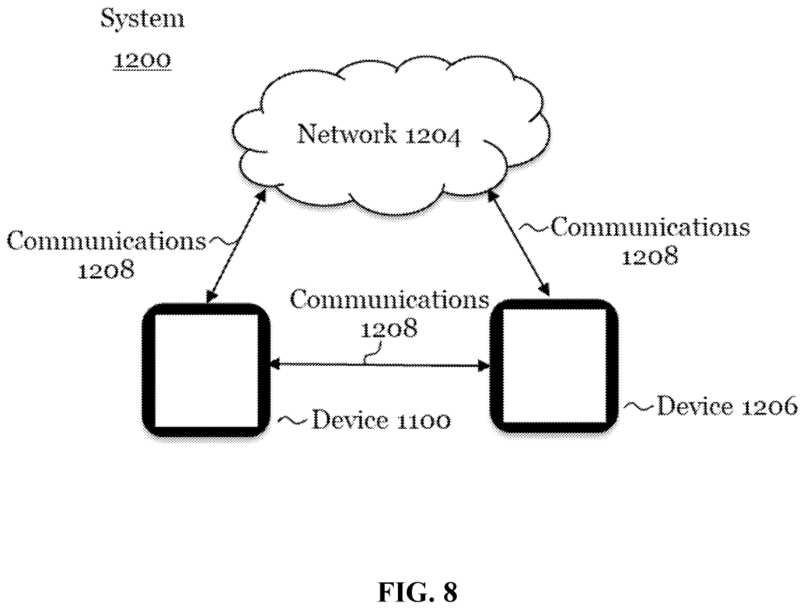

Device 1100 may be connected to a network (e.g., Network 1204, as shown in FIG. 8 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system (e.g., Operating System 1180) suitable for operating on the network. Detection module 1150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, Operating System 1180 is executed by one or more processors, e.g., Processor(s) 1110.

Device 1100 can further include Power Supply 1170, which can be any suitable power supply.

FIG. 8 illustrates an example of a computing system in accordance with one embodiment. In System 1200, Device 1100 (e.g., as described above and illustrated in FIG. 7) is connected to Network 1204, which is also connected to Device 1206. In some embodiments, Device 1206 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system. Devices 1100 and 1206 may communicate, e.g., using suitable communication interfaces via Network 1204, such as a Local Area Network (LAN), Virtual Private Network (VPN), or the Internet. In some embodiments, Network 1204 can be, for example, the Internet, an intranet, a virtual private network, a cloud network, a wired network, or a wireless network. Devices 1100 and 1206 may communicate, in part or in whole, via wireless or hardwired communications, such as Ethernet, IEEE 802.11b wireless, or the like. Additionally, Devices 1100 and 1206 may communicate, e.g., using suitable communication interfaces, via a second network, such as a mobile/cellular network. Communication between Devices 1100 and 1206 may further include or communicate with various servers such as a mail server, mobile server, media server, telephone server, and the like. In some embodiments, Devices 1100 and 1206 can communicate directly (instead of, or in addition to, communicating via Network 1204), e.g., via wireless or hardwired communications, such as Ethernet, IEEE 802.11b wireless, or the like. In some embodiments, Devices 1100 and 1206 communicate via Communications 1208, which can be a direct connection or can occur via a network (e.g., Network 1204).

One or all of Devices 1100 and 1206 generally include logic (e.g., http web server logic) or is programmed to format data, accessed from local or remote databases or other sources of data and content, for providing and/or receiving information via Network 1204 according to various examples described herein.

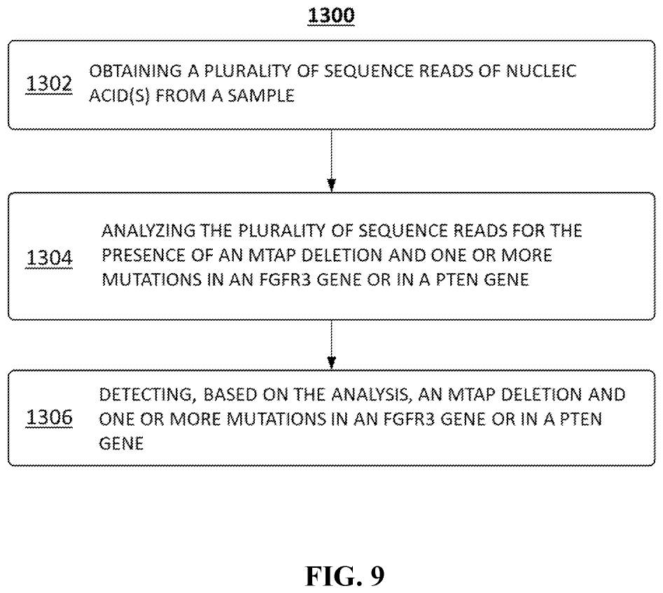

FIG. 9 illustrates an exemplary process 1300 for detecting an MTAP deletion and one or more mutations in an FGFR3 gene or in a PTEN gene, in accordance with some embodiments. Process 1300 is performed, for example, using one or more electronic devices implementing a software program. In some examples, process 1300 is performed using a client-server system, and the blocks of process 1300 are divided up in any manner between the server and a client device. In other examples, the blocks of process 1300 are divided up between the server and multiple client devices. Thus, while portions of process 1300 are described herein as being performed by particular devices of a client-server system, it will be appreciated that process 1300 is not so limited. In some embodiments, the executed steps can be executed across many systems, e.g., in a cloud environment. In other examples, process 1300 is performed using only a client device or only multiple client devices. In process 1300, some blocks are, optionally, combined, the order of some blocks is, optionally, changed, and some blocks are, optionally, omitted. In some examples, additional steps may be performed in combination with the process 1300. Accordingly, the operations as illustrated (and described in greater detail below) are exemplary by nature and, as such, should not be viewed as limiting.

At block 1302, a plurality of sequence reads of one or more nucleic acids is obtained, wherein the one or more nucleic acids are derived from a sample obtained from an individual. In some embodiments, the sequence reads are from a sequencer, e.g., as described herein or otherwise known in the art. In some embodiments, the nucleic acid(s) comprise one or more nucleic acids from a sample obtained from an individual having a urothelial bladder cancer (UCB). Optionally, prior to obtaining the sequence reads, the sample is purified, enriched, and/or subjected to PCR amplification. At block 1304, an exemplary system (e.g., one or more electronic devices) analyzes the plurality of sequence reads for the presence of an MTAP deletion and one or more mutations in an FGFR3 gene or in a PTEN gene. At block 1306, the system detects (e.g., based on the analysis) an MTAP deletion and one or more mutations in an FGFR3 gene or in a PTEN gene, in the sample. In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations result in one or more of an insertion, deletion or substitution of one or more amino acid residues in a polypeptide encoded by the FGFR3 gene or the PTEN gene. In some embodiments, the plurality of sequence reads is obtained by sequencing, whole exome sequencing, whole genome sequencing, gene-targeted sequencing, or next-generation sequencing.

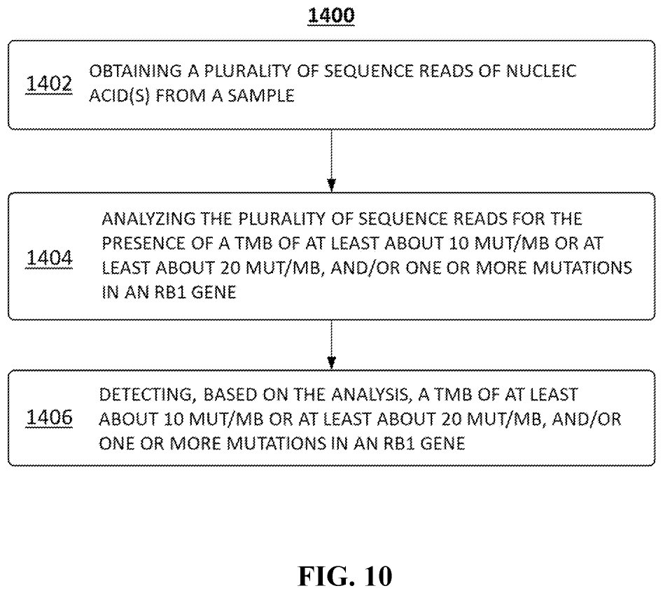

FIG. 10 illustrates an exemplary process 1400 for detecting a TMB of at least about 10 mut/Mb or at least about 20 mut/Mb, and/or one or more mutations in an RB1 gene, in accordance with some embodiments. Process 1400 is performed, for example, using one or more electronic devices implementing a software program. In some examples, process 1400 is performed using a client-server system, and the blocks of process 1400 are divided up in any manner between the server and a client device. In other examples, the blocks of process 1400 are divided up between the server and multiple client devices. Thus, while portions of process 1400 are described herein as being performed by particular devices of a client-server system, it will be appreciated that process 1400 is not so limited. In some embodiments, the executed steps can be executed across many systems, e.g., in a cloud environment. In other examples, process 1400 is performed using only a client device or only multiple client devices. In process 1400, some blocks are, optionally, combined, the order of some blocks is, optionally, changed, and some blocks are, optionally, omitted. In some examples, additional steps may be performed in combination with the process 1400. Accordingly, the operations as illustrated (and described in greater detail below) are exemplary by nature and, as such, should not be viewed as limiting.

At block 1402, a plurality of sequence reads of one or more nucleic acids is obtained, wherein the one or more nucleic acids are derived from a sample obtained from an individual. In some embodiments, the sequence reads are from a sequencer, e.g., as described herein or otherwise known in the art. In some embodiments, the nucleic acid(s) comprise one or more nucleic acids from a sample obtained from an individual having a urothelial bladder cancer (UCB). Optionally, prior to obtaining the sequence reads, the sample is purified, enriched, and/or subjected to PCR amplification. At block 1404, an exemplary system (e.g., one or more electronic devices) analyzes the plurality of sequence reads for the presence of a TMB of at least about 10 mut/Mb or at least about 20 mut/Mb, and/or one or more mutations in an RB1 gene. At block 1406, the system detects (e.g., based on the analysis) a TMB of at least about 10 mut/Mb or at least about 20 mut/Mb, and/or one or more mutations in an RB1 gene, in the sample. In some embodiments, the analyzing for the presence of a tumor mutational burden (TMB) is based on about 0.80 Mb of sequenced DNA. In some embodiments, the analyzing for the presence of a tumor mutational burden (TMB) is based on between about 0.83 Mb and about 1.14 Mb of sequenced DNA. In some embodiments, the analyzing for the presence of a tumor mutational burden (TMB) is based on up to about 1.1 Mb of sequenced DNA. In some embodiments, the one or more mutations in the RB1 gene comprise one or more of an insertion, deletion or substitution of one or more nucleotides, a genomic rearrangement, an alteration in a promoter of the RB1 gene, a gene fusion, or a copy number alteration. In some embodiments, the one or more mutations in the RB1 gene result in one or more of an insertion, deletion or substitution of one or more amino acid residues in a polypeptide encoded by the RB1 gene. In some embodiments, the plurality of sequence reads is obtained by sequencing, whole exome sequencing, whole genome sequencing, gene-targeted sequencing, or next-generation sequencing.

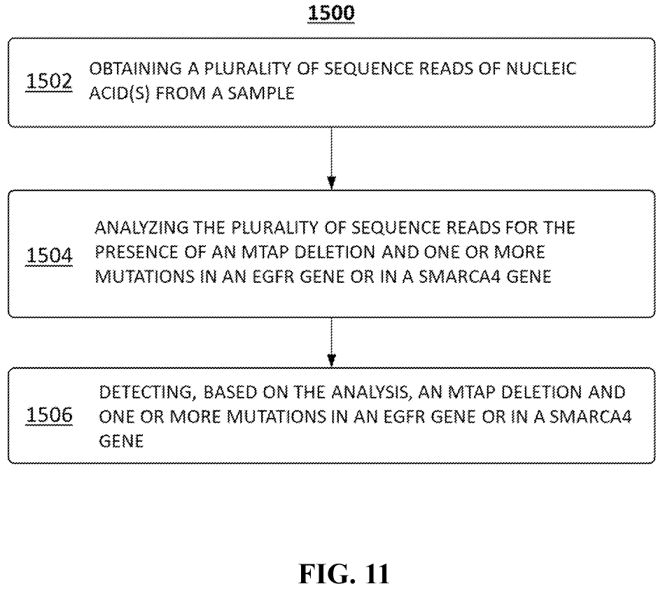

FIG. 11 illustrates an exemplary process 1500 for detecting an MTAP deletion, and one or more mutations in an EGFR gene or in a SMARCA4 gene, in accordance with some embodiments. Process 1500 is performed, for example, using one or more electronic devices implementing a software program. In some examples, process 1500 is performed using a client-server system, and the blocks of process 1500 are divided up in any manner between the server and a client device. In other examples, the blocks of process 1500 are divided up between the server and multiple client devices. Thus, while portions of process 1500 are described herein as being performed by particular devices of a client-server system, it will be appreciated that process 1500 is not so limited. In some embodiments, the executed steps can be executed across many systems, e.g., in a cloud environment. In other examples, process 1500 is performed using only a client device or only multiple client devices. In process 1500, some blocks are, optionally, combined, the order of some blocks is, optionally, changed, and some blocks are, optionally, omitted. In some examples, additional steps may be performed in combination with the process 1500. Accordingly, the operations as illustrated (and described in greater detail below) are exemplary by nature and, as such, should not be viewed as limiting.

At block 1502, a plurality of sequence reads of one or more nucleic acids is obtained, wherein the one or more nucleic acids are derived from a sample obtained from an individual. In some embodiments, the sequence reads are from a sequencer, e.g., as described herein or otherwise known in the art. In some embodiments, the nucleic acid(s) comprise one or more nucleic acids from a sample obtained from an individual having a non-small cell lung cancer (NSCLC). Optionally, prior to obtaining the sequence reads, the sample is purified, enriched, and/or subjected to PCR amplification. At block 1504, an exemplary system (e.g., one or more electronic devices) analyzes the plurality of sequence reads for the presence of an MTAP deletion, and one or more mutations in an EGFR gene or in a SMARCA4 gene. At block 1506, the system detects (e.g., based on the analysis) an MTAP deletion, and one or more mutations in an EGFR gene or in a SMARCA4 gene, in the sample. In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations result in one or more of an insertion, deletion or substitution of one or more amino acid residues in a polypeptide encoded by the EGFR gene or the SMARCA4 gene. In some embodiments, the plurality of sequence reads is obtained by sequencing, whole exome sequencing, whole genome sequencing, gene-targeted sequencing, or next-generation sequencing.

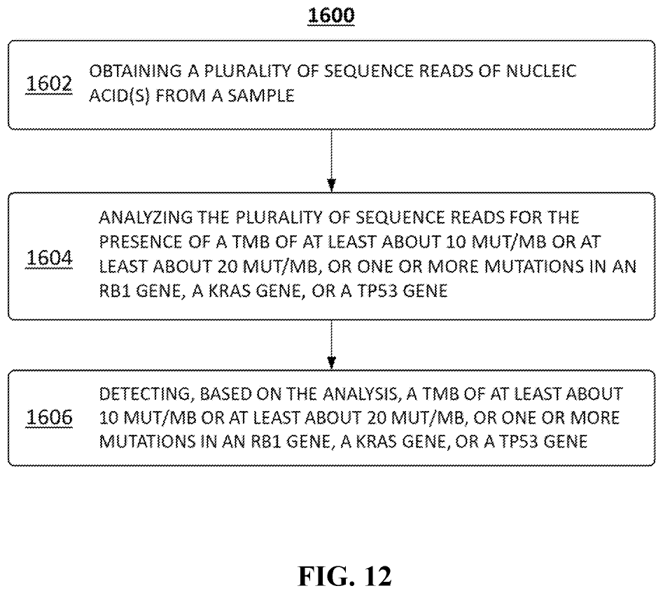

FIG. 12 illustrates an exemplary process 1600 for detecting a TMB of at least about 10 mut/Mb or at least about 20 mut/Mb, and/or one or more mutations in an RB1 gene, a KRAS gene, or a TP53 gene, in accordance with some embodiments. In some embodiments, the one or more mutations in the KRAS gene result in a G12C amino acid substitution in a KRAS polypeptide encoded by the KRAS gene. Process 1600 is performed, for example, using one or more electronic devices implementing a software program. In some examples, process 1600 is performed using a client-server system, and the blocks of process 1600 are divided up in any manner between the server and a client device. In other examples, the blocks of process 1600 are divided up between the server and multiple client devices. Thus, while portions of process 1600 are described herein as being performed by particular devices of a client-server system, it will be appreciated that process 1600 is not so limited. In some embodiments, the executed steps can be executed across many systems, e.g., in a cloud environment. In other examples, process 1600 is performed using only a client device or only multiple client devices. In process 1600, some blocks are, optionally, combined, the order of some blocks is, optionally, changed, and some blocks are, optionally, omitted. In some examples, additional steps may be performed in combination with the process 1600. Accordingly, the operations as illustrated (and described in greater detail below) are exemplary by nature and, as such, should not be viewed as limiting.

At block 1602, a plurality of sequence reads of one or more nucleic acids is obtained, wherein the one or more nucleic acids are derived from a sample obtained from an individual. In some embodiments, the sequence reads are from a sequencer, e.g., as described herein or otherwise known in the art. In some embodiments, the nucleic acid(s) comprise one or more nucleic acids from a sample obtained from an individual having a non-small cell lung cancer (NSCLC). Optionally, prior to obtaining the sequence reads, the sample is purified, enriched, and/or subjected to PCR amplification. At block 1604, an exemplary system (e.g., one or more electronic devices) analyzes the plurality of sequence reads for the presence of a TMB of at least about 10 mut/Mb or at least about 20 mut/Mb, and/or one or more mutations in an RB1 gene, a KRAS gene, or a TP53 gene. At block 1606, the system detects (e.g., based on the analysis) a TMB of at least about 10 mut/Mb or at least about 20 mut/Mb, and/or one or more mutations in an RB1 gene, a KRAS gene, or a TP53 gene, in the sample. In some embodiments, the one or more mutations in the KRAS gene result in a G12C amino acid substitution in a KRAS polypeptide encoded by the KRAS gene. In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations result in one or more of an insertion, deletion or substitution of one or more amino acid residues in a polypeptide encoded by the RB1 gene, KRAS gene, or TP53 gene. In some embodiments, the one or more mutations in the KRAS gene result in a G12C amino acid substitution in a KRAS polypeptide encoded by the KRAS gene. In some embodiments, the analyzing the plurality of sequence reads for the presence of a tumor mutational burden (TMB) of at least about 10 mut/Mb or at least about 20 mut/Mb is based on about 0.80 Mb of sequenced DNA. In some embodiments, the analyzing the plurality of sequence reads for the presence of a tumor mutational burden (TMB) of at least about 10 mut/Mb or at least about 20 mut/Mb is based on between about 0.83 Mb and about 1.14 Mb of sequenced DNA. In some embodiments, the analyzing the plurality of sequence reads for the presence of a tumor mutational burden (TMB) of at least about 10 mut/Mb or at least about 20 mut/Mb is based on about 1.1 Mb of sequenced DNA. In some embodiments, the analyzing the plurality of sequence reads for the presence of a tumor mutational burden (TMB) of at least about 10 mut/Mb or at least about 20 mut/Mb is based on up to about 1.1 Mb of sequenced DNA. In some embodiments, the plurality of sequence reads is obtained by sequencing, whole exome sequencing, whole genome sequencing, gene-targeted sequencing, or next-generation sequencing.

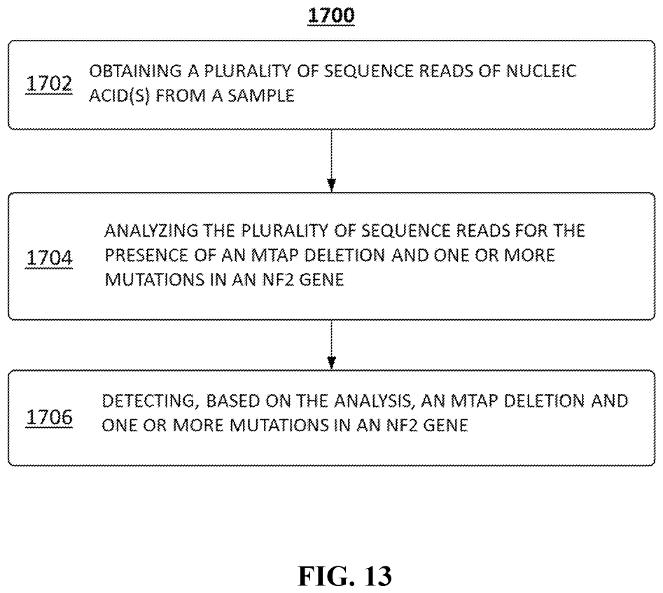

FIG. 13 illustrates an exemplary process 1700 for detecting an MTAP deletion and one or more mutations in an NF2 gene, in accordance with some embodiments. Process 1700 is performed, for example, using one or more electronic devices implementing a software program. In some examples, process 1700 is performed using a client-server system, and the blocks of process 1700 are divided up in any manner between the server and a client device. In other examples, the blocks of process 1700 are divided up between the server and multiple client devices. Thus, while portions of process 1700 are described herein as being performed by particular devices of a client-server system, it will be appreciated that process 1700 is not so limited. In some embodiments, the executed steps can be executed across many systems, e.g., in a cloud environment. In other examples, process 1700 is performed using only a client device or only multiple client devices. In process 1700, some blocks are, optionally, combined, the order of some blocks is, optionally, changed, and some blocks are, optionally, omitted. In some examples, additional steps may be performed in combination with the process 1700. Accordingly, the operations as illustrated (and described in greater detail below) are exemplary by nature and, as such, should not be viewed as limiting.

At block 1702, a plurality of sequence reads of one or more nucleic acids is obtained, wherein the one or more nucleic acids are derived from a sample obtained from an individual. In some embodiments, the sequence reads are from a sequencer, e.g., as described herein or otherwise known in the art. In some embodiments, the nucleic acid(s) comprise one or more nucleic acids from a sample obtained from an individual having a srcRCC or a ccRCC. Optionally, prior to obtaining the sequence reads, the sample is purified, enriched, and/or subjected to PCR amplification. At block 1704, an exemplary system (e.g., one or more electronic devices) analyzes the plurality of sequence reads for the presence of an MTAP deletion and one or more mutations in an NF2 gene. At block 1706, the system detects (e.g., based on the analysis) an MTAP deletion and one or more mutations in an NF2 gene, in the sample. In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations in the NF2 gene result in one or more of an insertion, deletion or substitution of one or more amino acid residues in a polypeptide encoded by the NF2 gene. In some embodiments, the plurality of sequence reads is obtained by sequencing, whole exome sequencing, whole genome sequencing, gene-targeted sequencing, or next-generation sequencing.

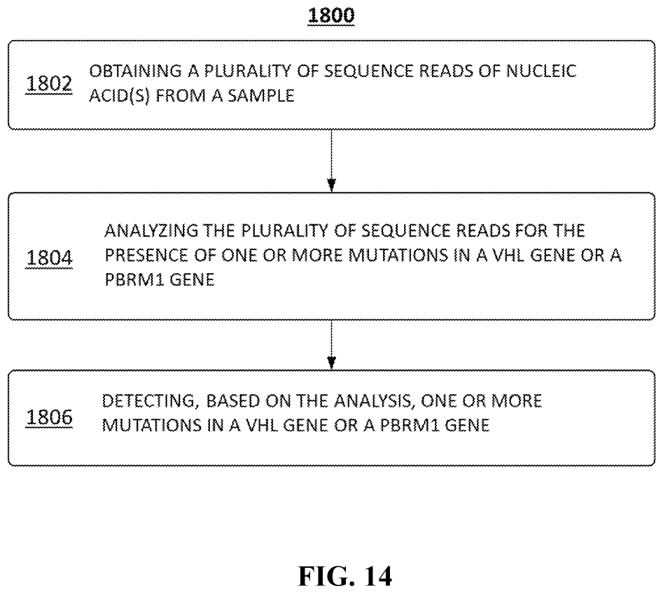

FIG. 14 illustrates an exemplary process 1800 for detecting one or more mutations in a VHL gene or a PBRM1 gene, in accordance with some embodiments. Process 1800 is performed, for example, using one or more electronic devices implementing a software program. In some examples, process 1800 is performed using a client-server system, and the blocks of process 1800 are divided up in any manner between the server and a client device. In other examples, the blocks of process 1800 are divided up between the server and multiple client devices. Thus, while portions of process 1800 are described herein as being performed by particular devices of a client-server system, it will be appreciated that process 1800 is not so limited. In some embodiments, the executed steps can be executed across many systems, e.g., in a cloud environment. In other examples, process 1800 is performed using only a client device or only multiple client devices. In process 1800, some blocks are, optionally, combined, the order of some blocks is, optionally, changed, and some blocks are, optionally, omitted. In some examples, additional steps may be performed in combination with the process 1800. Accordingly, the operations as illustrated (and described in greater detail below) are exemplary by nature and, as such, should not be viewed as limiting.

At block 1802, a plurality of sequence reads of one or more nucleic acids is obtained, wherein the one or more nucleic acids are derived from a sample obtained from an individual. In some embodiments, the sequence reads are from a sequencer, e.g., as described herein or otherwise known in the art. In some embodiments, the nucleic acid(s) comprise one or more nucleic acids from a sample obtained from an individual having a ccRCC. Optionally, prior to obtaining the sequence reads, the sample is purified, enriched, and/or subjected to PCR amplification. At block 1804, an exemplary system (e.g., one or more electronic devices) analyzes the plurality of sequence reads for the presence of one or more mutations in a VHL gene or a PBRM1 gene. At block 1806, the system detects (e.g., based on the analysis) one or more mutations in a VHL gene or a PBRM1 gene, in the sample. In some embodiments, the one or more mutations comprise one or more of an insertion, deletion or substitution of one or more nucleotides, a genomic rearrangement, an alteration in a promoter, a gene fusion, or a copy number alteration. In some embodiments, the one or more mutations in the VHL gene or PBRM1 gene result in one or more of an insertion, deletion or substitution of one or more amino acid residues in a polypeptide encoded by the VHL gene or PBRM1 gene. In some embodiments, the plurality of sequence reads is obtained by sequencing, whole exome sequencing, whole genome sequencing, gene-targeted sequencing, or next-generation sequencing.

The method steps of any of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### Antibodies

Provided herein are antibodies or antibody fragments for use in detecting one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703). In some embodiments, an antibody or antibody fragment of the disclosure specifically binds to a polypeptide encoded by a gene comprising one or more gene alterations of the disclosure, or a portion thereof. The antibody may be of any suitable type of antibody, including, but not limited to, a monoclonal antibody, a polyclonal antibody, a multi-specific antibody (e.g., a bispecific antibody), or an antibody fragment, so long as the antibody or antibody fragment exhibits a specific antigen binding activity (e.g., binding to a polypeptide encoded by a gene comprising one or more gene alterations of the disclosure).

In some embodiments, a polypeptide encoded by a gene comprising one or more gene alterations of the disclosure, or a fragment thereof, is used as an immunogen to generate one or more antibodies of the disclosure, e.g., using standard techniques for polyclonal and monoclonal antibody preparation. In some embodiments, a polypeptide encoded by a gene comprising one or more gene alterations of the disclosure, is used to provide antigenic peptide fragments (e.g., comprising any of at least about 8, at least about 10, at least about 15, at least about 20, at least about 30 or more amino acids) for use as immunogens to generate one or more antibodies of the disclosure, e.g., using standard techniques for polyclonal and monoclonal antibody preparation. As is known in the art, an antibody of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., a polypeptide encoded by a gene comprising one or more gene alterations of the disclosure, or a fragment thereof. The preparation can further include an adjuvant, such as Freund's complete or incomplete adjuvant, or a similar immunostimulatory agent.

In some embodiments, an antibody provided herein is a polyclonal antibody. Methods of producing polyclonal antibodies are known in the art. In some embodiments, an antibody provided herein is a monoclonal antibody, wherein a population of the antibody molecules contain only one species of an antigen binding site capable of immunoreacting or binding with a particular epitope, e.g., an epitope on a polypeptide encoded by a gene comprising one or more gene alterations of the disclosure. Methods of preparation of monoclonal antibodies are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody of the disclosure may also be identified and isolated by screening a recombinant combinatorial immunoglobulin library (e.g., an antibody phage display library) with the polypeptide of interest, e.g., a polypeptide encoded by a gene comprising one or more gene alterations of the disclosure, or a fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies of the disclosure are recombinant antibodies, such as chimeric and humanized monoclonal antibodies, comprising both human and non-human portions. Such chimeric and/or humanized monoclonal antibodies can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody of the disclosure is a human monoclonal antibody. In some embodiments, human monoclonal antibodies are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies, and protocols for producing such antibodies, see, e.g., U.S. Pat. Nos. 5,625,126; 5,633,425; 5,569,825; 5,661,016; and U.S. Pat. No. 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody of the disclosure can be used to isolate a polypeptide encoded by a gene comprising one or more gene alterations of the disclosure, or a fragment thereof, by standard techniques, such as affinity chromatography or immunoprecipitation. In some embodiments, an antibody of the disclosure can be used to detect a polypeptide encoded by a gene comprising one or more gene alterations of the disclosure, or a fragment thereof, e.g., in a tissue sample, cellular lysate, or cell supernatant, in order to evaluate the level and/or pattern of expression of the polypeptide. Detection can be facilitated by coupling the antibody to a detectable substance. Thus, in some embodiments, an antibody of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ¹²⁵I, ¹³¹I, ³⁵S or ³H.

An antibody or antibody fragment of the disclosure may also be used diagnostically, e.g., to detect and/or monitor protein levels (e.g., protein levels of a polypeptide encoded by a gene comprising one or more gene alterations of the disclosure) in tissues or body fluids (e.g., in a tumor cell-containing tissue or body fluid), e.g., according to the methods provided herein.

Antibody Affinity

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g., from 10⁻⁸ M to 10⁻¹³ M, e.g., from 10⁻⁹ M to 10⁻¹³ M). Methods of measuring antibody affinity (e.g., Kd) are known in the art, and include, without limitation, a radiolabeled antigen binding assay (RIA) and a BIACORE® surface plasmon resonance assay. In some embodiments, antibody affinity (e.g., Kd) is determined using the Fab version of an antibody of the disclosure and its antigen (e.g., a polypeptide encoded by a gene comprising one or more gene alterations of the disclosure). In some embodiments, a RIA is performed with the Fab version of an antibody of the disclosure and its antigen (e.g., a polypeptide encoded by a gene comprising one or more gene alterations of the disclosure).

Antibody Fragments

In certain embodiments, an antibody provided herein is an antibody fragment. Antibody fragments include, but are not limited to, Fab, Fab′, Fab′-SH, F(ab′)₂, Fv, and single-chain antibody molecules (e.g., scFv) fragments, and other fragments described herein.

In certain embodiments, an antibody provided herein is a diabody. Diabodies are antibody fragments with two antigen-binding sites that may be bivalent or bispecific. In certain embodiments, an antibody provided herein is a triabody or a tetrabody.

In certain embodiments, an antibody provided herein is a single-domain antibody. Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody, as well as production by recombinant host cells (e.g., E. coli or phage), as known in the art and as described herein.

Chimeric and Humanized Antibodies

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey), and a human constant region. In a further example, a chimeric antibody is a “class switched” antibody, in which the class or subclass of the antibody has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof), are derived from a non-human antibody, and framework regions (FRs) (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity. Humanized antibodies and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

Human Antibodies

In certain embodiments, an antibody provided herein is a human antibody. Human antibodies can be produced using various techniques known in the art. For example, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies can also be made by hybridoma-based methods known in the art, e.g., using known human myeloma and mouse-human heteromyeloma cell lines for the production of human monoclonal antibodies. Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Library-Derived Antibodies

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies to a wide range of non-self and also self antigens without any immunization. Naive libraries can also be made synthetically by cloning un-rearranged V-gene segments from stem cells, and using PCR primers containing random sequences to amplify the highly variable CDR3 regions and to accomplish rearrangement in vitro. Antibodies or antibody fragments isolated from human antibody libraries are considered human antibodies or human antibody fragments herein.

Multispecific Antibodies

In certain embodiments, an antibody provided herein is a multispecific antibody, e.g., a bispecific antibody. Multispecific antibodies are monoclonal antibodies that have binding specificities for at least two different sites or at least two different antigens. Multispecific antibodies can be prepared as full length antibodies or as antibody fragments. Techniques for making multispecific antibodies are known in the art and include, but are not limited to, recombinant co-expression of two immunoglobulin heavy chain-light chain pairs having different specificities, and “knob-in-hole” engineering. Multispecific antibodies may also be made by engineering electrostatic steering effects (e.g., by introducing mutations in the constant region) for making heterodimeric Fes; cross-linking two or more antibodies or fragments; using leucine zippers to produce bispecific antibodies; using “diabody” technology for making bispecific antibody fragments; using single-chain Fv (scFv) dimers; and preparing trispecific antibodies. Engineered antibodies with three or more functional antigen binding sites, including “Octopus antibodies,” are also included in the disclosure. Antibodies or antibody fragments of the disclosure also include “Dual Acting FAbs” or “DAF,” e.g., comprising an antigen binding site that binds to an immune checkpoint protein as well as another, different antigen.

Antibody Variants

In certain embodiments, amino acid sequence variants of the antibodies provided herein are contemplated. For example, it may be desirable to improve the binding affinity and/or other biological properties of the antibody. Amino acid sequence variants of an antibody of the disclosure may be prepared by introducing appropriate modifications into the nucleotide sequence encoding the antibody, or by peptide synthesis. Such modifications include, for example, deletions, and/or insertions, and/or substitutions of residues within the amino acid sequences of the antibody. Any combination of deletions, insertions, and substitutions can be made to arrive at the final antibody, provided that the final antibody possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody of the present disclosure is altered to increase or to decrease the extent to which the antibody is glycosylated. Addition or deletion of glycosylation sites to an antibody may be conveniently accomplished by altering the amino acid sequence of the antibody, such that one or more glycosylation sites is created or removed. Antibody variants having bisected oligosaccharides are further provided, e.g., in which a biantennary oligosaccharide attached to the Fc region of the antibody is bisected by GlcNAc. In some embodiments, antibody variants of the disclosure may have increased fucosylation. In some embodiments, antibody variants of the disclosure may have reduced fucosylation. In some embodiments, antibody variants of the disclosure may have improved ADCC function. In some embodiments, antibody variants of the disclosure may have decreased ADCC function. Antibody variants with at least one galactose residue in the oligosaccharide attached to the Fc region are also provided. Such antibody variants may have improved CDC function. In some embodiments, antibody variants of the disclosure may have increased CDC function. In some embodiments, antibody variants of the disclosure may have decreased CDC function.

In certain embodiments, one or more amino acid modifications may be introduced into the Fc region of an antibody of the present disclosure, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody variants with improved or diminished binding to FcRs are also included in the disclosure. In certain embodiments, an antibody variant comprises an Fc region with one or more amino acid substitutions that improve ADCC, e.g., substitutions at positions 298, 333, and/or 334 of the Fc region. In some embodiments, numbering of Fc region residues is according to EU numbering of residues. In some embodiments, alterations are made in the Fc region that result in altered (i.e., either improved or diminished) C1q binding and/or CDC. In some embodiments, antibodies of the disclosure include antibodies with increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody provided herein is a cysteine-engineered antibody, e.g., “thioMAb,” in which one or more residues of the antibody are substituted with cysteine residues. In some embodiments, the substituted residues occur at accessible sites of the antibody. By substituting those residues with cysteine, reactive thiol groups are thereby positioned at accessible sites of the antibody, and may be used to conjugate the antibody to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag. In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyamino acids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody may vary, and if more than one polymer is attached, the polymers can be the same or different molecules. In general, the number and/or type of polymers used for derivatization can be determined based on considerations including, but not limited to, the particular properties or functions of the antibody to be improved, or whether the antibody derivative will be used in a therapy under defined conditions. In some embodiments, provided herein are antibodies conjugated to carbon nanotubes, e.g., for use in methods to selectively heat the antibody using radiation to a temperature at which cells proximal to the antibody are killed.

### Anti-Cancer Therapies and Formulations

Certain aspects of the present disclosure relate to anti-cancer therapies. In some embodiments, an anti-cancer therapy of the disclosure includes one or more therapeutic agents, e.g., for treating a disease, disorder, or injury, e.g., a UCB, NSCLC, srcRCC or ccRCC associated with a deletion of an MTAP gene, or of a portion thereof, a TMB of at least about 10 mut/Mb or at least about 20 mut/Mb, a PD-L1-positive status, and/or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703.

In some embodiments, the anti-cancer therapy is a small molecule inhibitor, an antibody, a cellular therapy (i.e., a cell-based therapy), or a nucleic acid. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA). In some embodiments, the anti-cancer therapy is a chemotherapeutic agent, an anti-hormonal agent, an antimetabolite chemotherapeutic agent, a kinase inhibitor, a peptide, a gene therapy, a vaccine, a platinum-based chemotherapeutic agent, an immunotherapy, an antibody, or a checkpoint inhibitor. In some embodiments, the anti-cancer therapy is an FGFR-targeted therapy. In some embodiments, the anti-cancer therapy is a PTEN-targeted therapy. In some embodiments, the anti-cancer therapy is an RB1-targeted therapy. In some embodiments, the anti-cancer therapy is an EGFR-targeted therapy. In some embodiments, the anti-cancer therapy is a SMARCA4-targeted therapy. In some embodiments, the anti-cancer therapy is a TP53-targeted therapy. In some embodiments, the anti-cancer therapy is a KRAS-targeted therapy. In some embodiments, the anti-cancer therapy is a KRAS (G12C)-targeted therapy. In some embodiments, the anti-cancer therapy is an NF2-targeted therapy. In some embodiments, the anti-cancer therapy is a VHL-targeted therapy. In some embodiments, the anti-cancer therapy is a PBRM1-targeted therapy. In some embodiments, the anti-cancer therapy is an immunotherapy. In some embodiments, the anti-cancer therapy is a therapy targeted based on the presence of one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the cancer.

Anti-cancer therapies targeted to a particular gene (e.g., an FGFR-targeted therapy, an FGFR3-targeted therapy, a PTEN-targeted therapy, an RB1-targeted therapy, an EGFR-targeted therapy, a SMARCA4-targeted therapy, a TP53-targeted therapy, a KRAS-targeted therapy, a KRAS (G12C)-targeted therapy, an NF2-targeted therapy, a VHL-targeted therapy, or a PBRM1-targeted therapy) include any anti-cancer therapy (e.g., an anti-cancer therapy provided herein) that can provide a therapeutic benefit to an individual having cancer comprising one or more mutations in the targeted gene. Thus, targeted anti-cancer therapies provided herein (e.g., an FGFR-targeted therapy, an FGFR3-targeted therapy, a PTEN-targeted therapy, an RB1-targeted therapy, an EGFR-targeted therapy, a SMARCA4-targeted therapy, a TP53-targeted therapy, a KRAS-targeted therapy, a KRAS (G12C)-targeted therapy, an NF2-targeted therapy, a VHL-targeted therapy, or a PBRM1-targeted therapy) include anti-cancer therapies that exert their effects directly or indirectly on the targeted gene or a gene product encoded by the targeted gene (e.g., an RNA molecule or polypeptide), as well as anti-cancer therapies that do not exert their effects directly or indirectly on the targeted gene or a gene product encoded by the targeted gene (e.g., an RNA molecule or polypeptide), so long as the anti-cancer therapy can provide a therapeutic benefit to an individual having cancer comprising one or more mutations in the targeted gene. For example, an FGFR-targeted therapy or an FGFR3-targeted therapy may be any anti-cancer therapy (e.g., an anti-cancer therapy provided herein) that can provide a therapeutic benefit to an individual having cancer comprising one or more mutations in an FGFR3 gene; a PTEN-targeted therapy may be any anti-cancer therapy (e.g., an anti-cancer therapy provided herein) that can provide a therapeutic benefit to an individual having cancer comprising one or more mutations in a PTEN gene; an RB1-targeted therapy may be any anti-cancer therapy (e.g., an anti-cancer therapy provided herein) that can provide a therapeutic benefit to an individual having cancer comprising one or more mutations in an RB1 gene; an EGFR-targeted therapy may be any anti-cancer therapy (e.g., an anti-cancer therapy provided herein) that can provide a therapeutic benefit to an individual having cancer comprising one or more mutations in an EGFR gene; a SMARCA4-targeted therapy may be any anti-cancer therapy (e.g., an anti-cancer therapy provided herein) that can provide a therapeutic benefit to an individual having cancer comprising one or more mutations in a SMARCA4 gene; a TP53-targeted therapy may be any anti-cancer therapy (e.g., an anti-cancer therapy provided herein) that can provide a therapeutic benefit to an individual having cancer comprising one or more mutations in a TP53 gene; a KRAS-targeted therapy may be any anti-cancer therapy (e.g., an anti-cancer therapy provided herein) that can provide a therapeutic benefit to an individual having cancer comprising one or more mutations in a KRAS gene; a KRAS (G12C)-targeted therapy may be any anti-cancer therapy (e.g., an anti-cancer therapy provided herein) that can provide a therapeutic benefit to an individual having cancer comprising a KRAS G12C mutation; an NF2-targeted therapy may be any anti-cancer therapy (e.g., an anti-cancer therapy provided herein) that can provide a therapeutic benefit to an individual having cancer comprising one or more mutations in an NF2 gene; a VHL-targeted therapy may be any anti-cancer therapy (e.g., an anti-cancer therapy provided herein) that can provide a therapeutic benefit to an individual having cancer comprising one or more mutations in a VHL gene; a PBRM1-targeted therapy may be any anti-cancer therapy (e.g., an anti-cancer therapy provided herein) that can provide a therapeutic benefit to an individual having cancer comprising one or more mutations in a PBRM1 gene; and an anti-cancer therapy targeted to any of AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 may be any anti-cancer therapy (e.g., an anti-cancer therapy provided herein) that can provide a therapeutic benefit to an individual having cancer comprising one or more mutations in an AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 gene.

In some embodiments, the anti-cancer therapy comprises a second agent, such as a second anti-cancer agent. In some embodiments, the anti-cancer therapy is administered in combination with a second anti-cancer therapy or agent. In some embodiments, the anti-cancer therapy comprises a PRMT5-targeted therapy. In some embodiments, any of the anti-cancer therapies provided herein are administered in combination with a PRMT5-targeted therapy. In some embodiments, the anti-cancer therapy comprises an FGFR-targeted therapy and a PRMT5-targeted therapy. In some embodiments, the anti-cancer therapy comprises a PTEN-targeted therapy and a PRMT5-targeted therapy. In some embodiments, the anti-cancer therapy comprises an RB1-targeted therapy and a PRMT5-targeted therapy. In some embodiments, the anti-cancer therapy comprises an EGFR-targeted therapy and a PRMT5-targeted therapy. In some embodiments, the anti-cancer therapy comprises a SMARCA4-targeted therapy and a PRMT5-targeted therapy. In some embodiments, the anti-cancer therapy comprises a TP53-targeted therapy and a PRMT5-targeted therapy. In some embodiments, the anti-cancer therapy comprises a KRAS-targeted therapy and a PRMT5-targeted therapy. In some embodiments, the anti-cancer therapy comprises a KRAS (G12C)-targeted therapy and a PRMT5-targeted therapy. In some embodiments, the anti-cancer therapy comprises an NF2-targeted therapy and a PRMT5-targeted therapy. In some embodiments, the anti-cancer therapy comprises a VHL-targeted therapy and a PRMT5-targeted therapy. In some embodiments, the anti-cancer therapy comprises a PBRM1-targeted therapy and a PRMT5-targeted therapy. In some embodiments, the anti-cancer therapy comprises an immunotherapy and a PRMT5-targeted therapy. In some embodiments, the anti-cancer therapy comprises a therapy targeted based on the presence of one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703 in the cancer, and a PRMT5-targeted therapy.

In some embodiments, the FGFR-targeted therapy comprises one or more of a multi-kinase inhibitor, an FGFR-selective inhibitor, an FGFR3-specific inhibitor, or a combination therapy. In some embodiments, the multi-kinase inhibitor comprises one or more of E3810 (lucitanib), AZD4547, Dovitinib (TK1258), Ponatinib, Derazantinib (ARQ 087), Nintendanib (BIBF1120), Rogaratinib (BAY 1163877), 3D185, SOMCL-085, brivanib (BMS582664), lenvatinib (E7080), or orantinib (TSU-68). In some embodiments, the FGFR-selective inhibitor comprises one or more of PRN1371, XL-228, AZ12908010 (AZ8010), Debio-1347 (CH5183284), FIIN-2, LY2874455, Infigratinib (BGJ398, NVP-BGJ398), Pemigatinib (Pemazyre®, INCB054828), Erdafitinib (JNJ-42756493, Balversa®), ASP5878, TAS-120, PRN1371, or PKC412. In some embodiments, the FGFR3-specific inhibitor comprises one or more of Vofatamab (B-701, anti-FGFR3 antibody), or MFGR1877S (anti-FGFR3 antibody). In some embodiments, the combination therapy comprises one or more of: an FGFR-targeted therapy (e.g., an FGFR-targeted therapy provided herein) administered in combination with a PD-1/PD-L1-targeted therapy (e.g., a PD-1- or PD-L1-targeted therapy provided herein); an FGFR-targeted therapy (e.g., an FGFR-targeted therapy provided herein) administered in combination with an EGFR inhibitor (e.g., an EGFR inhibitor provided herein); an FGFR-targeted therapy (e.g., an FGFR-targeted therapy provided herein) administered in combination with an immunotherapy (e.g., an immunotherapy provided herein); an FGFR-targeted therapy (e.g., an FGFR-targeted therapy provided herein) administered in combination with a MAPK inhibitor (e.g., a MPAK inhibitor provided herein); an FGFR-targeted therapy (e.g., an FGFR-targeted therapy provided herein) administered in combination with a PI3K inhibitor (e.g., a PI3K inhibitor provided herein); an FGFR-targeted therapy (e.g., an FGFR-targeted therapy provided herein) administered in combination with an AKT inhibitor (e.g., an AKT inhibitor provided herein); or an FGFR-targeted therapy (e.g., an FGFR-targeted therapy provided herein) administered in combination with a VEGF inhibitor (e.g., a VEGF inhibitor provided herein). Additional information about FGFR-targeted therapies which may be used according to any of the methods provided herein, such as the targeted therapies provided herein, may be found in, e.g., clinicaltrials.gov/ct2/show/NCT02965378; Costa et al., Oncotarget (2016) 7(34):55924-55938; Ware et al., PLoS One (2010) 5(11):e14117; Katoh Int J Mol Med (2016) 38(1):3-15; clinicaltrials.gov/ct2/show/NCT04197986; clinicaltrials.gov/ct2/show/NCT01004224; Komla-Ebri et al., J Clin Invest (2016) 126(5):1871-84; clinicaltrials.gov/ct2/show/NCT02272998; clinicaltrials.gov/ct2/show/NCT04096417; Hall et al., PLoS One (2016) 11(9):e0162594; Lima et al., Int J Mol Sci (2020) 21(9):3214; Casadei et al., Therapeutic advances in medical oncology (2019) 11, 1758835919890285; Peng et al., J Exp Clin Cancer Res (2019) 38(1):372; Kikuchi et al., Cancer Sci (2017) 108(2):236-242; Facchinetti et al., Clinical cancer research: An official journal of the American Association for Cancer Research (2020) 26(4) 764-774; Jiang et al., Acta Pharmacol Sin (2017) 39(2):243-250; Touat et al., Clin Cancer Res (2015) 21(12):2684-94; and Chen et al., Oncogene (2005) 24(56):8259-67.

In some embodiments, the PTEN-targeted therapy comprises one or more of a PI3K inhibitor (e.g., a PI3K inhibitor provided herein), an AKT inhibitor (e.g., an AKT inhibitor provided herein), an mTOR inhibitor (e.g., an mTOR inhibitor provided herein), or a MET inhibitor (e.g., a MET inhibitor provided herein). In some embodiments, the PTEN-targeted therapy is administered in combination with an additional anti-cancer therapy, such as a hormone therapy agent (e.g., a hormone therapy agent provided herein), a chemotherapeutic agent (e.g., a chemotherapeutic agent provided herein), an mTOR inhibitor (e.g., an mTOR inhibitor provided herein), or an EGFR inhibitor (e.g., an EGFR inhibitor provided herein). Additional information about PTEN-targeted therapies which may be used according to any of the methods provided herein, such as the targeted therapies provided herein, may be found in, e.g., clinicaltrials.gov/ct2/show/NCT04493853; clinicaltrials.gov/ct2/show/NCT02761694; clinicaltrials.gov/ct2/show/NCT04251533; clinicaltrials.gov/ct2/show/NCT03218826; clinicaltrials.gov/ct2/show/NCT01430572; clinicaltrials.gov/ct2/show/NCT04439149; clinicaltrials.gov/ct2/show/NCT01870726; clinicaltrials.gov/ct2/show/NCT02822482; Pandolfi et al., PP. Engl J Med (2004) 351(22):2337-8; Nagata et al., Cancer Cell (2004) 6(2):117-27; Jiang and Liu, Adv Cancer Res (2009) 102:19-65; Haddadi et al., Mol Cancer (2018) 17:37; Jamaspishvili et al., Nat Rev Urol (2018) 15(4):222-234; and de Bono et al., Clin Cancer Res (2019) 25(3):928-936.

In some embodiments, the RB1-targeted therapy comprises one or more of a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor. In some embodiments, the chemotherapy is any chemotherapy or chemotherapeutic agent known in the art or provided herein. In some embodiments, the chemotherapy is a taxane. In some embodiments, the AURK inhibitor is LY3295668. In some embodiments, the CHK inhibitor comprises one or more of AZD7762, LY2603618 (prexatersib), PD-407824, SB-218078, NSC-109555, TCS-2312, UNC-01, XL-844, PF-00477736, PF-394691, or CHIR124. In some embodiments, the PLK inhibitor comprises one or more of Volasertib, B12536, HMN-214, GSK461364, BI 6727, ON09190, ON-019190.Na, NMS1, DAP-81, LC-445, CYC-800, ZK-thiazolidinone, Onvansertib (PCM-075), or CFI-400945. In some embodiments, the CDC25 inhibitor comprises one or more of NSC663284, BN82002, IRC-083864 (BN82685), IRC-083065, IRC-083842, NSC 95397, LGH00031, 5169131, SB-218078, NF201, NF336, dyasidiolide, sulfiricin, 8L, or NF339. In some embodiments, the AURK inhibitor comprises one or more of MK5108 (VX-689), LY3295668, ZM447439, hesperidin, VX-680 (tozasertib, MK0547), MLN8054, MLN-8237 (alisertib), PHA-680632, PHA-739358 (danusertib), JNJ-770621, SU6668, CCT129202, CCT137690, AZD2811 (barasertib), AT9283, SNS314, MP529, R763, ENMD2076, XL228, TTP607, PF-03814735, AMG-900, TAK-901, ENMD-2076, GSK10700916, or CYC116 (Cyclacel). In some embodiments, the EZH2 inhibitor comprises one or more of GSK126, EPZ6438, GSK343, EPZ005687, UNC1999, CPI-360, EI1, DZnep, or a stabilized a-helix of EZH2 peptide. Additional information about RB1-targeted therapies which may be used according to any of the methods provided herein, such as the targeted therapies provided herein, may be found in, e.g., Knudsen et al., Trends Cancer (2019) 5(5):308-324.

In some embodiments, the anti-cancer therapy comprises a SMARCA4-targeted therapy. In some embodiments, the SMARCA4-targeted therapy comprises one or more of a CDK4/6 inhibitor, an Aurora kinase (AURK) inhibitor, an ATR inhibitor, an EZH2 inhibitor, a KDM6 inhibitor, a kinase inhibitor, a cisplatin-based chemotherapy, or an immune checkpoint inhibitor. In some embodiments, the CDK4/6 inhibitor comprises one or more of palbociclib (S1116), abemaciclib (LY2835219), or ribociclib (LEE001). In some embodiments, the AURK inhibitor is VX-680. In some embodiments, the ATR inhibitor is VE822. In some embodiments, the EZH2 inhibitor comprises one or more of Tazemetostat (EPZ006438), GSK343, GSK126, UNC1999, EPZ-6438, or SAH-EZH2. In some embodiments, the KDM6 inhibitor is GSK-J4. In some embodiments, the kinase inhibitor is any kinase inhibitor known in the art or described herein. In some embodiments, the kinase inhibitor is ponatinib. In some embodiments, the cisplatin-based chemotherapy is any cisplatin-based chemotherapy known in the art or described herein. In some embodiments, the immune checkpoint inhibitor is any immune checkpoint inhibitor known in the art or described herein, such as nivolumab (anti-PD-1 antibody) or pembrolizumab (anti-PD-1 antibody). Additional information about SMARCA4-targeted therapies which may be used according to any of the methods provided herein, such as the targeted therapies provided herein, may be found in, e.g., Xue et al., Nat Commun (2019) 10, 557; Xue et al. Nat Commun (2019) 10, 558; Bell et al., Clin Cancer Res (2016)22(10):2396-404; Iijima et al., Immunotherapy (2020) 12(8):563-569; Naito et al., Thorac Cancer (2019) 10(5):1285-1288; Henon et al., Ann Oncol (2019) 30(8):1401-1403; Tagal et al., (2017) Nat Commun 8, 14098; Kiminori et al., NAR Cancer (2020) vol. 2, issue 2; Chan-Penebre et al., Mol Cancer Ther (2017) 16(5):850-860; Knutson et al., Mol Cancer Ther (2015) 14 (12 Supplement 2) C87; Lang et al., Clin Cancer Res (2018) 24(8):1932-1943; Kim et al., Nature medicine (2016) 22(2) 128-134.

In some embodiments, the anti-cancer therapy comprises a TP53-targeted therapy. In some embodiments, the TP53-targeted therapy comprises one or more of a p53 reactivator, an mTOR inhibitor, a Pin1 inhibitor, an ATR inhibitor, a proteasome inhibitor, a CHK inhibitor, an ATM inhibitor, a WEE1 inhibitor, or a murine double minute 2 (MDM2) inhibitor. In some embodiments, the TP53-targeted therapy is a combination therapy comprising: a WEE1 inhibitor and an HDAC inhibitor; an ATM inhibitor and a PARP inhibitor; a p53 reactivator and a proteasome inhibitor; a p53 reactivator and an MDM2 inhibitor; an HSP90 inhibitor and an HDAC inhibitor; a PI3K inhibitor and a p53 reactivator; a PARP inhibitor and a p53 reactivator; a p53 reactivator and a cisplatin-based therapy; or a PARP inhibitor and a CHK1 inhibitor. In some embodiments, the p53 reactivator comprises one or more of PRIMA-1, APR-246, PK11007, COTI-2, NSC319726 (ZMC1), Reacp53, RETRA, P53R3, PK083, SCH529074, PK7088, Stictic acid, chetomin, CP-31398, STIMA-1, 3-Benzoylacrylic acid, or KSS-9. In some embodiments, the HDAC inhibitor is vorinostat. In some embodiments, the mTOR inhibitor is everolimus. In some embodiments, the MDM2 inhibitor comprises one or more of 05045337 (RG7112; Nutlin-3), MI-219, MI-319, Deazaflavin 5 (HLI-393), arylsulfonamide, bisarylurea, acylimidazolone, JNJ-26854165, TDP521252, TDP665759, or RITA (NSC652287). In some embodiments, the proteasome inhibitor is carbilozib. In some embodiments, the Pin1 inhibitor comprises one or more of ATRA, ATO, or KPT-6566. In some embodiments, the ATM inhibitor is AZD0156. In sme embodiments, the WEE1 inhibitor is AZD1775 (MK-1775). In some embodiments, the ATR inhibitor comprises one or more of BAY1895344, AZD6738 and VX-970 (VE-822, berzosertib). In some embodiments, the CHK inhibitor comprises one or more of AZD7762, MK-8776 (SCH 900776), LY2603618 (Rabusertib), LY2606368 (Prexasertib), GDC-0425, or SAR-020106. Additional information about TP53-targeted therapies which may be used according to any of the methods provided herein, such as the targeted therapies provided herein, may be found in, e.g., Duffy et al., Breast Cancer Res Treat (2018) 170(2):213-219; Gupta et al., Biomed Pharmacother (2019) 109:484-492; Bykov et al., Nat Rev Cancer (2018) 18(2):89-102; Lindemann et al., Journal of dental research (2018) 97(6), 635-644; and Mantovani et al., Cell death and differentiation (2019) 26(2), 199-212.

In some embodiments, the anti-cancer therapy comprises an EGFR-targeted therapy. In some embodiments, the EGFR-targeted therapy comprises one or more of an EGFR inhibitor, an HSP90 inhibitor, a VEGFR/EGFR dual inhibitor, a MEK inhibitor, or a Raf inhibitor. In some embodiments, the EGFR-targeted therapy comprises a combination of: a MAPK inhibitor and an EGFR inhibitor; an mTOR inhibitor and an EGFR inhibitor; or a p100alpha inhibitor and an EGFR inhibitor. In some embodiments, the mTOR inhibitor comprises one or more of sirolimus, temsirolimus, everolimus, or AP235732. In some embodiments, the EGFR inhibitor comprises one or more of lapatinib, erlotinib, gefitinib, cetuximab, trastuzumab (Herceptin), mAb806 (anti-EGFR antibody), panitunumab, AZD9291 (osimertinib), CO-1686 (rociletinib), EAI045, icotinib, BIBW 2992 (afatinib), PF00299804 (dacomitinib), HKI-272 (neratinib), CI-1033 (canertinib), AZD9291 (osimertinib), HM61713 (olmutinib), CO-1686 (rociletinib), ASP8273 or EGF816. In some embodiments, the HSP90 inhibitor is IPI-504 and/or 17-AAG. In some embodiments, the VEGFR/EGFR dual inhibitor comprises one or more of ZD6464, AEE788163, or XL647. In some embodiments, the MEK inhibitor is PD-325901. In some embodiments, the Raf inhibitor is Sorafenib (BAY49-9006). In some embodiments, the P100alpha inhibitor is PX-866. Additional information about EGFR-targeted therapies which may be used according to any of the methods provided herein, such as the targeted therapies provided herein, may be found in, e.g., Sebastian et al., Eur Respir Rev (2014) 23(131):92-105; Koustas et al., Cancer Lett (2017) 396:94-102; Wang et al., Cancer Lett (2017) 385:51-54; Lee et al., Expert Opin Investig Drugs (2014) 23(10):1333-48; Sharma et al., Nat Rev Cancer (2007) 7(3):169-81; Liu et al., Oncotarget (2017) 8(30) 50209-50220; and Rajaram et al., Indian J Dent Res (2017) 28(6):687-694.

In some embodiments, the KRAS-targeted therapy comprises a kinase inhibitor. In some embodiments, the kinase inhibitor is MCP1, SCH54292, tipifarnib (R115777), lonafarnib (SCH663366), or BMS-214662. In some embodiments, the KRAS-targeted therapy comprises one or more of a farnesyltransferase inhibitor, a geranylgeranyltransferase inhibitor, a palmitoylation inhibitor, an inhibitor of methylation cleavage, or an agent that inhibits the modification or post-translational processing of KRAS. In other embodiments, the KRAS-targeted therapy comprises one or more of a Raf inhibitor, a MEK inhibitor, or an mTOR inhibitor. In some embodiments, the KRAS-targeted therapy comprises a KRAS (G12C)-targeted therapy. In some embodiments, the KRAS (G12C)-targeted therapy comprises one or more of KRAS inhibitor and/or a SHP2 (also known as PTPN11) inhibitor. In some embodiments, the KRAS (G12C)-targeted therapy is a combination therapy comprising a KRAS inhibitor and a HER family inhibitor; a KRAS inhibitor and a SHP2 inhibitor; a KRAS inhibitor and an mTOR inhibitor; a KRAS inhibitor and a CDK4/6 inhibitor; a KRAS inhibitor and an IGFR1 inhibitor; an mTOR inhibitor and an IGFR1 inhibitor; a KRAS inhibitor, an mTOR inhibitor and an IGFR1 inhibitor; a KRAS inhibitors and a PD-1 inhibitor or PD-L1 inhibitor; a pan-KRAS inhibitor and a MEK inhibitor; a SHP2 inhibitor and a MEK inhibitor; or a KRAS inhibitor, a PD-1 inhibitor or PD-L1 inhibitor, and a HER family inhibitor. In some embodiments, the KRAS inhibitor is a KRAS (G12C) inihibitor. In some embodiments, the KRAS inhibitor comprises one or more of Adagrasib (MRTX849), AMG 510 (sotorasib), ARS-1620, ARS853, JNJ-74699157 (ARS-3248), LY3499446, GDC-6036, or JDQ443. In some embodiments, the HER2 family inhibitor comprises one or more of afatinib, erlotinib, cetuximab, or bevacizumab. In some embodiments, the SHP2 inhibitor comprises one or more of RMC-4550, TNO155, or RMC-4630. In some embodiments, the mTOR inhibitor comprises one or more of vistusertib, everolimus, and/or AZD8055. In some embodiments, the CDK4/6 inhibitor comprises abemaciclib and/or palbociclib. In some embodiments, the IGFR1 inhibitor comprises linsitinib (OSI-906). In some embodiments, the PD-1 or PD-L1 inhibitor comprises pembroluzimab, spartaziluma, and/or atezolizumab. In some embodiments, the pan-KRAS inhibitor comprises BI-1701963. In some embodiments, the MEK inhibitor comprises cobimetinib and/or trametinib. In some embodiments, the KRAS-targeted therapy comprises one or more of Adagrasib (MRTX849), AMG 510 (sotorasib), ARS-1620, ARS853, JNJ-74699157 (ARS-3248), LY3499446, GDC-6036, JDQ443. In some embodiments, the KRAS-targeted therapy comprises BI-1701963. Additional information about KRAS-targeted therapies which may be used according to any of the methods provided herein, such as the targeted therapies provided herein, may be found in, e.g., clinicaltrials.gov/ct2/show/NCT03785249; Janne et al., European Journal of Cancer (2020) 138, S1-S2; clinicaltrials.gov/ct2/show/NCT03600883; Romero et al., Nat Rev Clin Oncol (2020) 17, 6; Hallin et al., Cancer Discov (2020) 10(1):54-71; Janes et al., Cell (2018) 172(3):578-589.ei7; Lito et al., Science (2016) 351(6273), 604-608; Hallin et al., Cancer Discov (2020) 10(1):54-71; Molina-Arca et al., Science translational medicine (2019) 11(510); Nagasaka et al., Cancer treatment reviews (2020) 84, 101974; clinicaltrials.gov/ct2/show/NCT03785249; Friday et al., Biochim Biophys Acta (2005) 1756:127-44; Holderfield Cold Spring Harb Perspect Med (2018) 8(7):a031864; Adjei J Thorac Oncol (2008) 3 (6 Suppl 2):5160-31; and Uprety and Adjei Cancer Treat Rev (2020) 89:102070.

In some embodiments, the VHL-targeted therapy comprises one or more of a poly(ADP-ribose) polymerase (PARP) inhibitor, a glutaminase 1 (GLS-1) inhibitor, a VEGF inhibitor, a HIF-2alpha inhibitor, an HDAC inhibitor, a CDK4/6 inhibitor, a Tank binding kinase 1 (TBK1) inhibitor, an EZH1 and/or EZH2 inhibitor, a Rho-Associated Kinase 1 (ROCK1) inhibitor, a glucose transporter 1 (GLUT1) inhibitor, an autophagy modulator, or an immune checkpoint inhibitor. In some embodiments, the VHL-targeted therapy is a combination therapy comprising a HIF-2alpha inhibitor and a CDK4/6 inhibitor; or a VEGF inhibitor and a PARP inhibitor. In some embodiments, the PARP inhibitors is olaparib and/or BMN-673. In some embodiments, the GLS-1 inhibitor is bis-2-(5-phenylacetamido-1,3,4-thiadiazol-2-yl)ethyl sulfide (BPTES). In some embodiments, the TBK inhibitor is compound 1, e.g., as described in Hu et al., Cancer Discov (2020) 10(3):460-475. In some embodiments, the VEGF inhibitor is pazopanib and/or cediranib. In some embodiments, the HIF-2alpha inhibitor comprises one or more of PT-2399, PT-2385, or PT-2977 (MK-6482, Beltuzifan). In some embodiments, the HDAC inhibitor is Vorinostat (SAHA). In some embodiments, the CDK4/6 inhibitor is Palbociclib (S1116) and/or abemaciclib (LY2835219). In some embodiments, the TBK1 inhibitor comprises one or more of UNC6587, MRT67307, or BX795. In some embodiments, the EZH1 and/or EZH2 inhibitor is JQ-EZ-05 and/or GSK-126. In some embodiments, the ROCK1 inhibitor is RKI 1447, GSK429286 and/or Y-27632. In some embodiments, the GLUT1 inhibitor is STF-31. In some embodiments, the autophagy modulators is STF-62247. Additional information about VHL-targeted therapies which may be used according to any of the methods provided herein, such as the targeted therapies provided herein, may be found in, e.g., Okazaki et al., J Clin Invest (2017) 127(5):1631-1645; Kidney Cancer Journal (2020) vol. 18, number 1; www.kidney-cancer-journal.com/assets/kcj_v18n1_final_lo-res_2.5.pdf#page=6; clinicaltrials.gov/ct2/show/NCT03108066; clinicaltrials.gov/ct2/show/NCT03401788; clinicaltrials.gov/ct2/show/NCT02108002; Nicholson et al., Science signaling (2019) 12(601), eaay0482; Scanlon et al., Oncotarget (2017) 9(4), 4647-4660; Hu et al., Cancer Discov (2020) 10(3):460-475; Turcotte et al., Cancer cell (2008) 14(1), 90-102; Chakraborty et al., Science translational medicine (2017) 9(398), eaa15272; Thompsonet et al., Oncogene (2017) 36(8), 1080-1089; and Chan et al., Science translational medicine (2011) 3(94), 94ra70.

In some embodiments, the anti-cancer therapy comprises a PBRM1-targeted therapy. In some embodiments, the PBRM1-targeted therapy comprises one or more of an EZH2 inhibitor, a VEGF inhibitor, or an mTOR inhibitor. In some embodiments, the PBRM1-targeted therapy is a combination therapy comprising a PARP inhibitor and an ATR inhibitor. In some embodiments, the EZH2 inhibitor is Tazemetostat, GSK126, and/or L501-1669. In some embodiments, the VEGF inhibitor is Sutinitib and/or pazopanib. In some embodiments, the mTOR inhibitor is everolimus. Additional information about PBRM1-targeted therapies which may be used according to any of the methods provided herein, such as the targeted therapies provided herein, may be found in, e.g., Huang et al., Cell Cycle (2020) 19(7):758-771; Chabanon et al., Cancer Res (2021) PMID: 33888468; and Carril-Ajuria et al., Cancers (Basel) (2019) 12(1):16.

In some embodiments, the anti-cancer therapy comprises an NF2-targeted therapy. In some embodiments, the NF2-targeted therapy comprises one or more of an mTOR inhibitor, a VEGF inhibitor, a focal adhesion kinase (FAK) inhibitor, an EGFR inhibitor, a NEDD8-activating enzyme (NAE) inhibitor, a MET inhibitor, a MEK inhibitor, a SRC inhibitor, a JNK inhibitor, a CDK inhibitor, a WEE1, a CHK1 inhibitor, or a multi-targeted kinase inhibitor. In some embodiments, the NF2-targeted therapy is a combination therapy comprising an NAE inhibitor and an mTOR inhibitor; or a TRK inhibitor and an mTOR inhibitor. In some embodiments, the mTOR inhibitor comprises one or more of AZD2014, VT3989, AZD201, INK128, GDC-0980, or everolimus. In some embodiments, the VEGF inhibitor comprises one or more of PTC299 or bevacizumab. In some embodiments, the FAK inhibitor comprises one or more of NVP-226, Y15, PND-1186, PF562 (271-26, PF-271), PF 573228 (PF-228), FRAX597, TAE226, PF-00562271, or GSK2256098 (vismodegib). In some embodiments, the EGFR inhibitor comprises lapatinib. In some embodiments, the NAE inhibitor comprises MLN4924. In some embodiments, the MET inhibitor comprises PHA-665752. In some embodiments, the MEK inhibitor comprises trametinib and/or AZD6244. In some embodiments, the SRC inhibitor comprises one or more of Saracatanib, dasatanib, PP2, or WH-4-023. In some embodiments, the JNK inhibitor comprises JNK-9L. In some embodiments, the CDK inhibitor comprises PD-0332991. In some embodiments, the WEE1/CHK1 inhibitor comprises 6881640. In some embodiments, the multi-targeted kinase inhibitor comprises sunitinib. Additional information about NF2-targeted therapies which may be used according to any of the methods provided herein, such as the targeted therapies provided herein, may be found in, e.g., clinicaltrials.gov/ct2/show/NCT04665206; clinicaltrials.gov/ct2/show/NCT02352844; clinicaltrials.gov/ct2/show/NCT02523014; Nunes et al., PLoS One (2013) 8(3):e59941; Shah et al., Gynecologic oncology (2014) 134(1), 104-111; Schroeder et al., Oncotarget (2014) 5(1), 67-77; Cooper et al., Mol Cancer Ther (2017) 16(8):1693-1704, Erratum in: Mol Cancer Ther. 202;20(2):450; Zhao et al., Proc Natl Acad Sci USA (2018) 115(9):E2077-E2084; Garcia-Rendueles et al., Cancer Discov (2015) 5(11):1178-93, Erratum in: Cancer Discov. 2019;9(11):1628; Angus et al., Neuro Oncol (2018) 2;20(9):1185-1196; Petrilli et al., Oncogene (2016) 35(5), 537-548; Sourbier et al., Oncotarget (2018) 9(12), 10723-10733; and Dunn et al., Anti-cancer agents in medicinal chemistry (2010) 10(10) 722-734.

In some embodiments, the anti-cancer therapy comprises a PRMT5-targeted therapy. PRMT5-targeted therapies include any anti-cancer therapy (e.g., an anti-cancer therapy provided herein) that exerts its effects directly or indirectly on a PRMT5 gene or gene product (e.g., an RNA molecule or polypeptide), or on a cellular pathway that is regulated or affected by a PRMT5 gene or gene product (e.g., an RNA molecule or polypeptide). In some embodiments, the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437. In some embodiments, the PRMT5-targeted therapy comprises a PRMT1 inhibitor. In some embodiments, the PRMT1 inhibitor comprises one or more of GSK3368715 (EPZ019997), DB75 (furamidine), E84, allantodapsone, stilbamidine, AMI-1, NS-1, A36, MI-21, DCLX078, DCLX069, RM65, AMI-8, or MS023. Additional information about PMRT5-targeted therapies which may be used according to any of the methods provided herein, such as the targeted therapies provided herein, may be found in, e.g., Zheng et al. Theranostics (2019) 9(9):2606-2617; Jin et al., The Journal of clinical investigation (2016) 126(10):3961-3980; Stopa et al., Cellular and molecular life sciences (CMLS) (2015) 72(11):2041-2059; Penebre et al., Blood (2014) 124 (21):438; Webb et al., J Immunol (2017) 198(4):1439-1451; Xiao et al., Biomed Pharmacother (2019) 114:108790; Smith et al., Expert Opin Ther Targets (2018) 22(6):527-545; Rugo et al., Advances in therapy (2020) 37(7):3059-3082; Webb et al., Journal of immunology (2017) 198(4):1439-1451; and Fedoriw et al., Cell stress (2020) 4(8):199-215.

In some embodiments, the anti-cancer therapy comprises a CDKN2A-targeted therapy. In some embodiments, the CDKN2A-targeted therapy comprises one or more of a CDK4/6 inhibitor, an Aurora kinase (AURK) inhibitor, a WEE1 inhibitor, a checkpoint kinase (CHK) inhibitor, or a mouse double minute 2 homolog (MDM2) inhibitor. In some embodiments, the CDKN2A-targeted therapy is a combination therapy comprising a CDK4/6 inhibitor and a VEGF inhibitor. In some embodiments, the CDK4/6 inhibitor comprises one or more of palbociclib (S1116, Ibrance®, PD0332991), abemaciclib (LY2835219), or ribociclib (LEE001, Kisqali®). In some embodiments, the AURK inhibitor is Ilorasertib (ABT348). In some embodiments, the ATR inhibitor is VE822. In some embodiments, the WEE1 inhibitor is AZD1775. In some embodiments, the CHK inhibitor is prexasertib. In some embodiments, the MDM2 inhibitor is any MDM2 inhibitor known in the art or described herein. In some embodiments, the VEGF inhibitor is bevacizumab. Additional information about CDKN2A-targeted therapies, such as the targeted therapies provided herein, may be found in, e.g., clinicaltrials.gov/ct2/show/NCT03389477; clinicaltrials.gov/ct2/show/NCT02478320; clinicaltrials.gov/ct2/show/NCT02540876; clinicaltrials.gov/ct2/show/NCT04074785; clinicaltrials.gov/ct2/show/NCT03356223; clinicaltrials.gov/ct2/show/NCT02688907; Gadhikar et al., (2018) Cancer Res. 78(3):781-797; Iacobucci et al., (2011) Clin Cancer Res. 17(23):7413-23; Lee et al., (2015) Curr Opin Oncol. 27(2):141-50; and Gonzilez-Gil et al., (2021) Genes 12(1):79.

In some embodiments, the anti-cancer therapy comprises a CDKN2B-targeted therapy. In some embodiments, the CDKN2B-targeted therapy comprises one or more of a CDK4/6 inhibitor, a checkpoint kinase (CHK) inhibitor, or a mouse double minute 2 homolog (MDM2) inhibitor. In some embodiments, the CDKN2B-targeted therapy is a combination therapy comprising a CDK4/6 inhibitor and a VEGF inhibitor. In some embodiments, the CDK4/6 inhibitor comprises one or more of palbociclib (S1116, Ibrance®, PD0332991), abemaciclib (LY2835219), or ribociclib (LEE001, Kisqali®). In some embodiments, the CHK inhibitor is prexasertib. In some embodiments, the MDM2 inhibitor is any MDM2 inhibitor known in the art or described herein. In some embodiments, the VEGF inhibitor is bevacizumab. Additional information about CDKN2B-targeted therapies, such as the targeted therapies provided herein, may be found in, e.g., Gonzilez-Gil et al., (2021) Genes 12(1):79; Iacobucci et al., (2011) Clin Cancer Res. 17(23):7413-23; Gadhikar et al., (2018) Cancer Res. 78(3):781-797; and clinicaltrials.gov/ct2/show/NCT04074785.

In some embodiments, the anti-cancer therapy comprises a PI3K inhibitor. In some embodiments, the PI3K inhibitor is GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), GS-9820, GSK2636771, GDC-0980, AMG319, pazobanib, or alpelisib (BYL719, Piqray).

In some embodiments, the anti-cancer therapy comprises an MAT2A inhibitor. In some embodiments, the MAT2A inhibitor comprises one or more of AG-270, AGI-24512, PF-9366, acetyl-11-keto-β-boswellic acid (AKBA), or fluorinated N,N-dialkylaminostilbene-5 (FIDAS).

In some embodiments, the anti-cancer therapy comprises an AKT inhibitor. In some embodiments, the AKT inhibitor is capivasertib, ARQ751, API-59-OMe, perifosine (KRX-0401), A-443654, PX-316, ipatasertib (GDC-0068), or MK2206.

In some embodiments, the anti-cancer therapy comprises an mTOR inhibitor. In some embodiments, the mTOR inhibitor is AP-23573, everolimus (RAD001), or CCI-779.

In some embodiments, the anti-cancer therapy comprises a MET inhibitor. In some embodiments, the MET inhibitor is capmatinib (INC280).

In some embodiments, the anti-cancer therapy comprises a heat shock protein (HSP) inhibitor, a MYC inhibitor, an HDAC inhibitor, an immunotherapy, a neoantigen, a vaccine, or a cellular therapy.

In some embodiments, the second anti-cancer agent includes one or more of an immune checkpoint inhibitor, a chemotherapy, a VEGF inhibitor, an Integrin $33 inhibitor, a statin, an EGFR inhibitor, an mTOR inhibitor, a PI3K inhibitor, a MAPK inhibitor, or a CDK4/6 inhibitor.

In some embodiments, the anti-cancer therapy comprises a kinase inhibitor. In some embodiments, the methods provided herein comprise administering to the individual a kinase inhibitor, e.g., in combination with another anti-cancer therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). Examples of ALK kinase inhibitors that may be used according to any of the methods provided herein are described in examples 3-39 of WO2005016894, which is incorporated herein by reference.

In some embodiments, the anti-cancer therapy comprises a heat shock protein (HSP) inhibitor. In some embodiments, the methods provided herein comprise administering to the individual an HSP inhibitor, e.g., in combination with another anti-cancer therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor. In some embodiments, the methods provided herein comprise administering to the individual a MYC inhibitor, e.g., in combination with another anti-cancer therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor. In some embodiments, the methods provided herein comprise administering to the individual an HDAC inhibitor, e.g., in combination with another anti-cancer therapy. In some embodiments, the HDAC inhibitor is belinostat (PXD101, Beleodaq®), SAHA (vorinostat, suberoylanilide hydroxamine, Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor. In some embodiments, the methods provided herein comprise administering to the individual a VEGF inhibitor, e.g., in combination with another anti-cancer therapy. In some embodiments, the VEGF inhibitor is Bevacizumab (Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin $3 inhibitor. In some embodiments, the methods provided herein comprise administering to the individual an integrin $3 inhibitor, e.g., in combination with another anti-cancer therapy. In some embodiments, the integrin $3 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor. In some embodiments, the methods provided herein comprise administering to the individual an αIIbβ3 integrin inhibitor, e.g., in combination with another anti-cancer therapy. In some embodiments, the αIIbβ3 integrin inhibitor is abciximab, eptifibatide (Integrilin®), or tirofiban (Aggrastat®).

In some embodiments, the anti-cancer therapy comprises a statin or a statin-based agent. In some embodiments, the methods provided herein comprise administering to the individual a statin or a statin-based agent, e.g., in combination with another anti-cancer therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises an mTOR inhibitor. In some embodiments, the methods provided herein comprise administering to the individual an mTOR inhibitor, e.g., in combination with another anti-cancer therapy. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a PI3K inhibitor. In some embodiments, the methods provided herein comprise administering to the individual a PI3K inhibitor, e.g., in combination with another anti-cancer therapy. In some embodiments, the PI3K inhibitor is GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), duvelisib, GS-9820, GSK2636771, GDC-0980, AMG319, pazobanib, or alpelisib (BYL719, Piqray).

In some embodiments, the anti-cancer therapy comprises a MAPK inhibitor. In some embodiments, the methods provided herein comprise administering to the individual a MAPK inhibitor, e.g., in combination with another anti-cancer therapy. In some embodiments, the MAPK inhibitor is SB203580, SKF-86002, BIRB-796, SC-409, RJW-67657, BIRB-796, VX-745, R03201195, SB-242235, orMW181.

In some embodiments, the anti-cancer therapy comprises a CDK4/6 inhibitor. In some embodiments, the methods provided herein comprise administering to the individual a CDK4/6 inhibitor, e.g., in combination with another anti-cancer therapy. In some embodiments, the CDK4/6 inhibitor is ribociclib (Kisqali®, LEE011), palbociclib (PD0332991, Ibrance®), or abemaciclib (LY2835219).

In some embodiments, the anti-cancer therapy comprises an EGFR inhibitor. In some embodiments, the methods provided herein comprise administering to the individual an EGFR inhibitor, e.g., in combination with another anti-cancer therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., in combination with another anti-cancer therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA that encodes a neoantigen, e.g., a neoantigen expressed by a cancer of the disclosure. In some embodiments, the cancer vaccine comprises RNA that encodes a neoantigen, e.g., a neoantigen expressed by a cancer of the disclosure. In some embodiments, the cancer vaccine comprises a polynucleotide that encodes a neoantigen, e.g., a neoantigen expressed by a cancer of the disclosure, as well as one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-lh68/GLV-lh153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; or DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics), a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy or immunotherapy comprises an immune checkpoint inhibitor. In some embodiments, the methods provided herein comprise administering to the individual an immune checkpoint inhibitor, e.g., in combination with another anti-cancer therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIRI, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIRI, CD160, 2B4, TGFRbeta, A2AR, GITR (CD357), CD80 (B7-1), CD86 (B7-2), CD276 (B7-H3), VTCN1 (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSFi4, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is selected from the group consisting of MDX-1 106 (nivolumab), MK-3475 (pembrolizumab, Keytruda®), MEDI-0680 (AMP-514), PDR001, REGN2810, MGA-012, JNJ-63723283, BI 754091, and BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANBO11; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANBO11), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IVIM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (1B1310, BMS-734016, MDXO10, MDX-CTLA4, MED14736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MED14736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine. In some embodiments, the methods provided herein comprise administering to the individual an immunoregulatory molecule or a cytokine, e.g., in combination with another anti-cancer therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3a (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groa, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises an anti-cancer agent that inhibits expression of a nucleic acid molecule or polypeptide. In some embodiments, the methods provided herein comprise administering to the individual an anti-cancer agent that inhibits expression of a nucleic acid molecule or polypeptide, e.g., in combination with another anti-cancer therapy.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a nucleic acid molecule. In some embodiments, a dsRNA, an siRNA, or an shRNA of the disclosure comprises a nucleotide sequence that is configured to hybridize to the breakpoint of a fusion nucleic acid molecule provided herein. Methods for designing, optimizing, producing, and using dsRNAs, siRNAs, or shRNAs, are known in the art.

In some embodiments, the anti-cancer therapy comprises a chemotherapy. In some embodiments, the methods provided herein comprise administering to the individual a chemotherapy, e.g., in combination with another anti-cancer therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor. In some embodiments, the methods provided herein comprise administering to the individual a kinase inhibitor, e.g., in combination with another anti-cancer therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-3 and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina vβ3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

Formulations to be used for in vivo administration are sterile. This is readily accomplished by filtration through sterile filtration membranes or other methods known in the art.

In some embodiments, the anti-cancer therapy is administered as a monotherapy. In some embodiments, the anti-cancer therapy is administered in combination with one or more additional anti-cancer therapies or treatments. In some embodiments, the one or more additional anti-cancer therapies or treatments include one or more anti-cancer therapies described herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy.

In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

Any of the anti-cancer therapies (optionally as monotherapies or in combination with another therapy or treatment) may find use in any of the methods described herein.

### Kits

Also provided herein are kits for detecting a deletion of an MTAP gene, a CDKN2A gene, and/or a CDKN2B gene, or portions thereof, and/or one or more mutations in one or more genes selected from TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, in a sample from an individual having a cancer, e.g., a UCB. In some embodiments, a kit provided herein comprises a reagent (e.g., one or more oligonucleotides, primers, probes or baits of the present disclosure) for detecting a deletion of an MTAP gene, a CDKN2A gene, and/or a CDKN2B gene, or portions thereof, and one or more mutations in one or more genes selected from TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1. In some embodiments, the kit comprises a reagent (e.g., one or more oligonucleotides, primers, probes or baits of the present disclosure) for detecting a wild-type counterpart of the MTAP, CDKN2A, CDKN2B, TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1 genes. In some embodiments, the reagent comprises one or more oligonucleotides, primers, probes or baits of the present disclosure capable of hybridizing to a nucleic acid molecule comprising or encoding a deletion of an MTAP gene, a CDKN2A gene, or a CDKN2B gene, or portions thereof, or to a wild-type counterpart. In some embodiments, the reagent comprises one or more oligonucleotides, primers, probes or baits of the present disclosure capable of hybridizing to a nucleic acid molecule comprising or encoding one or more mutations in a gene selected from TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, or to a wild-type counterpart. In some embodiments, the reagent comprises one or more oligonucleotides, primers, probes or baits of the present disclosure capable of distinguishing a nucleic acid molecule comprising or encoding a deletion of an MTAP gene, a CDKN2A gene, or a CDKN2B gene, or portions thereof, or one or more mutations in a gene selected from TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, from a corresponding wild-type counterpart of the nucleic acid molecule. In some embodiments, the kit is for use according to any method of detecting gene alterations in nucleic acid molecules known in the art or described herein, such as sequencing, next-generation sequencing, PCR, in situ hybridization methods, a nucleic acid hybridization assay, an amplification-based assay, a PCR-RFLP assay, real-time PCR, sequencing, a screening analysis, FISH, spectral karyotyping, MFISH, comparative genomic hybridization, in situ hybridization, sequence-specific priming (SSP) PCR, HPLC, or mass-spectrometric genotyping. In some embodiments, a kit provided herein further comprises instructions for detecting a deletion of an MTAP gene, a CDKN2A gene, and/or a CDKN2B gene, or portions thereof, and one or more mutations in one or more genes selected from TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, e.g., using one or more oligonucleotides, primers, probes or baits of the present disclosure.

Also provided herein are kits for detecting a polypeptide of the disclosure, e.g., a polypeptide encoded by a gene comprising one or more alterations (e.g., a deletion of an MTAP gene, a CDKN2A gene, and/or a CDKN2B gene, or portions thereof, or one or more mutations in one or more genes selected from TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1). In some embodiments, a kit provided herein comprises a reagent (e.g., one or more antibodies of the present disclosure) for detecting a polypeptide encoded by a gene comprising one or more alterations of the disclosure. In some embodiments, the kit comprises a reagent (e.g., one or more antibodies of the present disclosure) for detecting the wild-type counterparts of a polypeptide encoded by a gene comprising one or more alterations of the disclosure. In some embodiments, the reagent comprises one or more antibodies of the present disclosure capable of binding to a polypeptide encoded by a gene comprising one or more alterations of the disclosure, or to wild-type counterparts of the polypeptide. In some embodiments, the reagent comprises one or more antibodies of the present disclosure capable of distinguishing a polypeptide encoded by a gene comprising one or more alterations of the disclosure from wild-type counterparts of the polypeptide. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), or hyperdiffusion chromatography). In some embodiments, the kit further comprises instructions for detecting a polypeptide encoded by a gene comprising one or more alterations of the disclosure, e.g., using one or more antibodies of the present disclosure.

Also provided herein are kits for detecting a TMB of at least about 10 mut/Mb or at least about 20 mut/Mb, a PD-L1-positive status, and/or one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, in a sample from an individual having a cancer, e.g., a UCB. In some embodiments, a kit provided herein comprises a reagent (e.g., one or more oligonucleotides, primers, probes or baits of the present disclosure) for detecting a TMB of at least about 10 mut/Mb or at least about 20 mut/Mb, a PD-L1-positive status, and/or one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the kit comprises a reagent (e.g., one or more oligonucleotides, primers, probes or baits of the present disclosure) for detecting a wild-type counterpart of one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the reagent comprises one or more oligonucleotides, primers, probes or baits of the present disclosure capable of hybridizing to a nucleic acid molecule comprising or encoding one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, or to a wild-type counterpart. In some embodiments, the reagent comprises one or more oligonucleotides, primers, probes or baits of the present disclosure capable of distinguishing a nucleic acid molecule comprising or encoding one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, from a corresponding wild-type counterpart of the nucleic acid molecule. In some embodiments, the kit is for use according to any method of detecting or measuring TMB, PD-L1 expression, or one or more gene alterations in nucleic acid molecules known in the art or described herein, such as sequencing, exome sequencing, whole genome sequencing, gene-targeted sequencing, next-generation sequencing, PCR, in situ hybridization methods, a nucleic acid hybridization assay, an amplification-based assay, a PCR-RFLP assay, real-time PCR, sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting a TMB of at least about 10 mut/Mb or at least about 20 mut/Mb, a PD-L1-positive status, and/or one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, e.g., using one or more oligonucleotides, primers, probes or baits of the present disclosure.

In some embodiments, a kit provided herein comprises one or more reagents (e.g., one or more oligonucleotides, primers, probes or baits of the present disclosure) for detecting or measuring the level of TMB in a sample, e.g., in a sample from an individual having a UCB described herein. In some embodiments, the kit is for use according to any method for detecting or measuring TMB known in the art or provided herein, e.g., whole-exome sequencing (WES), next-generation sequencing, whole genome sequencing, gene-targeted sequencing, or sequencing of a panel of genes, e.g., panels including cancer-related genes.

In some embodiments, a kit provided herein comprises one or more reagents (e.g., one or more oligonucleotides, primers, antibodies, probes or baits of the present disclosure) for detecting a PD-L1-positive status in a sample, e.g., in a sample from an individual having a UCB described herein. In some embodiments, the kit is for use according to any method for detecting a PD-L1-positive status known in the art or provided herein, e.g. using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used. In some embodiments, the kit is suitable for use in detecting a PD-L1-positive status in sample using a VENTANA PD-L1 assay (SP142). In some embodiments, the kit comprises an anti-PD-L1 antibody suitable for use in detecting a PD-L1-positive status in sample using a VENTANA PD-L1 assay (SP142). In some embodiments, the kit is suitable for use in detecting a PD-L1-positive status in sample using a DAKO 22C3 assay. In some embodiments, the kit comprises an anti-PD-L1 antibody suitable for use in detecting a PD-L1-positive status in sample using a DAKO 22C3 assay.

Also provided herein are kits for detecting a polypeptide of the disclosure, e.g., a polypeptide encoded by a gene comprising one or more alterations (e.g., in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2). In some embodiments, a kit provided herein comprises a reagent (e.g., one or more antibodies of the present disclosure) for detecting a polypeptide encoded by a gene comprising one or more alterations of the disclosure, e.g., one or more mutations in one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2. In some embodiments, the kit comprises a reagent (e.g., one or more antibodies of the present disclosure) for detecting the wild-type counterparts of a polypeptide encoded by a gene, e.g., one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, comprising one or more alterations of the disclosure. In some embodiments, the reagent comprises one or more antibodies of the present disclosure capable of binding to a polypeptide encoded by a gene, e.g., one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, comprising one or more alterations of the disclosure or to wild-type counterparts of the polypeptide. In some embodiments, the reagent comprises one or more antibodies of the present disclosure capable of distinguishing a polypeptide encoded by a gene, e.g., one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, comprising one or more alterations of the disclosure from wild-type counterparts of the polypeptide. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a polypeptide encoded by a gene, e.g., one or more genes selected from TERT, TP53, RB1, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2, comprising one or more alterations of the disclosure, e.g., using one or more antibodies of the present disclosure.

Also provided herein are kits for detecting a deletion of an MTAP gene, a CDKN2A gene, and/or a CDKN2B gene, or portions thereof, and/or one or more mutations in one or more genes selected from TP53, KRAS, EGFR, ALK, ROS1, NTRK1, STK11, KEAP1, PIK3CA, SMARCA4, PTEN, MET, ERBB2, BRAF, or RB1, in a sample from an individual having a cancer, e.g., a NSCLC. In some embodiments, a kit provided herein comprises a reagent (e.g., one or more oligonucleotides, primers, probes or baits of the present disclosure) for detecting a deletion of an MTAP gene, a CDKN2A gene, and/or a CDKN2B gene, or portions thereof, and one or more mutations in one or more genes selected from TP53, KRAS, EGFR, ALK, ROS1, NTRK1, STK11, KEAP1, PIK3CA, SMARCA4, PTEN, MET, ERBB2, BRAF, or RB1. In some embodiments, the kit comprises a reagent (e.g., one or more oligonucleotides, primers, probes or baits of the present disclosure) for detecting a wild-type counterpart of the MTAP, CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1 genes. In some embodiments, the reagent comprises one or more oligonucleotides, primers, probes or baits of the present disclosure capable of hybridizing to a nucleic acid molecule comprising or encoding a deletion of an MTAP gene, a CDKN2A gene, or a CDKN2B gene, or portions thereof, or to a wild-type counterpart. In some embodiments, the reagent comprises one or more oligonucleotides, primers, probes or baits of the present disclosure capable of hybridizing to a nucleic acid molecule comprising or encoding one or more mutations in a gene selected from TP53, KRAS, EGFR, ALK, ROS1, NTRK1, STK11, KEAP1, PIK3CA, SMARCA4, PTEN, MET, ERBB2, BRAF, or RB1, or to a wild-type counterpart. In some embodiments, the reagent comprises one or more oligonucleotides, primers, probes or baits of the present disclosure capable of distinguishing a nucleic acid molecule comprising or encoding a deletion of an MTAP gene, a CDKN2A gene, or a CDKN2B gene, or portions thereof, or one or more mutations in a gene selected from TP53, KRAS, EGFR, ALK, ROS1, NTRK1, STK11, KEAP1, PIK3CA, SMARCA4, PTEN, MET, ERBB2, BRAF, or RB1, from a corresponding wild-type counterpart of the nucleic acid molecule. In some embodiments, the kit is for use according to any method of detecting gene alterations in nucleic acid molecules known in the art or described herein, such as sequencing, next-generation sequencing, PCR, in situ hybridization methods, a nucleic acid hybridization assay, an amplification-based assay, a PCR-RFLP assay, real-time PCR, sequencing, a screening analysis, FISH, spectral karyotyping, MFISH, comparative genomic hybridization, in situ hybridization, sequence-specific priming (SSP) PCR, HPLC, or mass-spectrometric genotyping. In some embodiments, a kit provided herein further comprises instructions for detecting a deletion of an MTAP gene, a CDKN2A gene, and/or a CDKN2B gene, or portions thereof, and one or more mutations in one or more genes selected from TP53, KRAS, EGFR, ALK, ROS1, NTRK1, STK11, KEAP1, PIK3CA, SMARCA4, PTEN, MET, ERBB2, BRAF, or RB1, e.g., using one or more oligonucleotides, primers, probes or baits of the present disclosure.

Also provided herein are kits for detecting a polypeptide of the disclosure, e.g., a polypeptide encoded by a gene comprising one or more alterations (e.g., a deletion of an MTAP gene, a CDKN2A gene, and/or a CDKN2B gene, or portions thereof, or one or more mutations in one or more genes selected from TP53, KRAS, EGFR, ALK, ROS1, NTRK1, STK11, KEAP1, PIK3CA, SMARCA4, PTEN, MET, ERBB2, BRAF, or RB1). In some embodiments, a kit provided herein comprises a reagent (e.g., one or more antibodies of the present disclosure) for detecting a polypeptide encoded by a gene comprising one or more alterations of the disclosure. In some embodiments, the kit comprises a reagent (e.g., one or more antibodies of the present disclosure) for detecting the wild-type counterparts of a polypeptide encoded by a gene comprising one or more alterations of the disclosure. In some embodiments, the reagent comprises one or more antibodies of the present disclosure capable of binding to a polypeptide encoded by a gene comprising one or more alterations of the disclosure, or to wild-type counterparts of the polypeptide. In some embodiments, the reagent comprises one or more antibodies of the present disclosure capable of distinguishing a polypeptide encoded by a gene comprising one or more alterations of the disclosure from wild-type counterparts of the polypeptide. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), or hyperdiffusion chromatography). In some embodiments, the kit further comprises instructions for detecting a polypeptide encoded by a gene comprising one or more alterations of the disclosure, e.g., using one or more antibodies of the present disclosure.

Also provided herein are kits for detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, in a sample from an individual having a cancer, e.g., a NSCLC. In some embodiments, a kit provided herein comprises a reagent (e.g., one or more oligonucleotides, primers, probes or baits of the present disclosure) for detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the kit comprises a reagent (e.g., one or more oligonucleotides, primers, probes or baits of the present disclosure) for detecting a wild-type counterpart of one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the reagent comprises one or more oligonucleotides, primers, probes or baits of the present disclosure capable of hybridizing to a nucleic acid molecule comprising or encoding one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, or to a wild-type counterpart. In some embodiments, the reagent comprises one or more oligonucleotides, primers, probes or baits of the present disclosure capable of distinguishing a nucleic acid molecule comprising or encoding one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, from a corresponding wild-type counterpart of the nucleic acid molecule. In some embodiments, the kit is for use according to any method of detecting or measuring TMB, PD-L1 expression, or one or more gene alterations in nucleic acid molecules known in the art or described herein, such as sequencing, exome sequencing, whole genome sequencing, gene-targeted sequencing, next-generation sequencing, PCR, in situ hybridization methods, a nucleic acid hybridization assay, an amplification-based assay, a PCR-RFLP assay, real-time PCR, sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting a TMB of at least about 10 mut/Mb or at least about 20 mut/Mb, a PD-L1-positive status, and/or one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, e.g., using one or more oligonucleotides, primers, probes or baits of the present disclosure.

In some embodiments, a kit provided herein comprises one or more reagents (e.g., one or more oligonucleotides, primers, probes or baits of the present disclosure) for detecting or measuring the level of TMB in a sample, e.g., in a sample from an individual having a NSCLC described herein. In some embodiments, the kit is for use according to any method for detecting or measuring TMB known in the art or provided herein, e.g., whole-exome sequencing (WES), next-generation sequencing, whole genome sequencing, gene-targeted sequencing, or sequencing of a panel of genes, e.g., panels including cancer-related genes.

In some embodiments, a kit provided herein comprises one or more reagents (e.g., one or more oligonucleotides, primers, antibodies, probes or baits of the present disclosure) for detecting a PD-L1-positive status in a sample, e.g., in a sample from an individual having a NSCLC described herein. In some embodiments, the kit is for use according to any method for detecting a PD-L1-positive status known in the art or provided herein, e.g. using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used. In some embodiments, the kit is suitable for use in detecting a PD-L1-positive status in sample using a VENTANA PD-L1 assay (SP142). In some embodiments, the kit comprises an anti-PD-L1 antibody suitable for use in detecting a PD-L1-positive status in sample using a VENTANA PD-L1 assay (SP142). In some embodiments, the kit is suitable for use in detecting a PD-L1-positive status in sample using a DAKO 22C3 assay. In some embodiments, the kit comprises an anti-PD-L1 antibody suitable for use in detecting a PD-L1-positive status in sample using a DAKO 22C3 assay.

Also provided herein are kits for detecting a polypeptide of the disclosure, e.g., a polypeptide encoded by a gene comprising one or more alterations (e.g., in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1). In some embodiments, a kit provided herein comprises a reagent (e.g., one or more antibodies of the present disclosure) for detecting a polypeptide encoded by a gene comprising one or more alterations of the disclosure, e.g., one or more mutations in one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1. In some embodiments, the kit comprises a reagent (e.g., one or more antibodies of the present disclosure) for detecting the wild-type counterparts of a polypeptide encoded by a gene, e.g., one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprising one or more alterations of the disclosure. In some embodiments, the reagent comprises one or more antibodies of the present disclosure capable of binding to a polypeptide encoded by a gene, e.g., one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprising one or more alterations of the disclosure or to wild-type counterparts of the polypeptide. In some embodiments, the reagent comprises one or more antibodies of the present disclosure capable of distinguishing a polypeptide encoded by a gene, e.g., one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprising one or more alterations of the disclosure from wild-type counterparts of the polypeptide. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a polypeptide encoded by a gene, e.g., one or more genes selected from CDKN2A, CDKN2B, TP53, KRAS, EGFR, ALK, ROS1, NTRK1, STK11, KEAP1, PIK3CA, SMARCA4, PTEN, MET, ERBB2, BRAF, or RB1, comprising one or more alterations of the disclosure, e.g., using one or more antibodies of the present disclosure.

Also provided herein are kits for detecting a deletion of an MTAP gene, a CDKN2A gene, and/or a CDKN2B gene, or portions thereof, and/or one or more mutations in one or more genes selected from VHL, PBRM1, SETD2, BAP1, TP53, PTEN, TERT, TSC1, PIK3CA, ATM, NF2, or MET, in a sample from an individual having a cancer, e.g., a srcRCC or a ccRCC. In some embodiments, a kit provided herein comprises a reagent (e.g., one or more oligonucleotides, primers, probes or baits of the present disclosure) for detecting a deletion of an MTAP gene, a CDKN2A gene, and/or a CDKN2B gene, or portions thereof, and one or more mutations in one or more genes selected from VHL, PBRM1, SETD2, BAP1, TP53, PTEN, TERT, TSC1, PIK3CA, ATM, NF2, or MET. In some embodiments, the kit comprises a reagent (e.g., one or more oligonucleotides, primers, probes or baits of the present disclosure) for detecting a wild-type counterpart of the MTAP, CDKN2A, CDKN2B, VHL, PBRM1, SETD2, BAP1, TP53, PTEN, TERT, TSC1, PIK3CA, ATM, NF2, or MET genes. In some embodiments, the reagent comprises one or more oligonucleotides, primers, probes or baits of the present disclosure capable of hybridizing to a nucleic acid molecule comprising or encoding a deletion of an MTAP gene, a CDKN2A gene, or a CDKN2B gene, or portions thereof, or to a wild-type counterpart. In some embodiments, the reagent comprises one or more oligonucleotides, primers, probes or baits of the present disclosure capable of hybridizing to a nucleic acid molecule comprising or encoding one or more mutations in a gene selected from VHL, PBRM1, SETD2, BAP1, TP53, PTEN, TERT, TSC1, PIK3CA, ATM, NF2, or MET, or to a wild-type counterpart. In some embodiments, the reagent comprises one or more oligonucleotides, primers, probes or baits of the present disclosure capable of distinguishing a nucleic acid molecule comprising or encoding a deletion of an MTAP gene, a CDKN2A gene, or a CDKN2B gene, or portions thereof, or one or more mutations in a gene selected from VHL, PBRM1, SETD2, BAP1, TP53, PTEN, TERT, TSC1, PIK3CA, ATM, NF2, or MET, from a corresponding wild-type counterpart of the nucleic acid molecule. In some embodiments, the kit is for use according to any method of detecting gene alterations in nucleic acid molecules known in the art or described herein, such as sequencing, next-generation sequencing, PCR, in situ hybridization methods, a nucleic acid hybridization assay, an amplification-based assay, a PCR-RFLP assay, real-time PCR, sequencing, a screening analysis, FISH, spectral karyotyping, MFISH, comparative genomic hybridization, in situ hybridization, sequence-specific priming (SSP) PCR, HPLC, or mass-spectrometric genotyping. In some embodiments, a kit provided herein further comprises instructions for detecting a deletion of an MTAP gene, a CDKN2A gene, and/or a CDKN2B gene, or portions thereof, and one or more mutations in one or more genes selected from VHL, PBRM1, SETD2, BAP1, TP53, PTEN, TERT, TSC1, PIK3CA, ATM, NF2, or MET, e.g., using one or more oligonucleotides, primers, probes or baits of the present disclosure.

Also provided herein are kits for detecting a polypeptide of the disclosure, e.g., a polypeptide encoded by a gene comprising one or more alterations (e.g., a deletion of an MTAP gene, a CDKN2A gene, and/or a CDKN2B gene, or portions thereof, or one or more mutations in one or more genes selected from VHL, PBRM1, SETD2, BAP1, TP53, PTEN, TERT, TSC1, PIK3CA, ATM, NF2, or MET). In some embodiments, a kit provided herein comprises a reagent (e.g., one or more antibodies of the present disclosure) for detecting a polypeptide encoded by a gene comprising one or more alterations of the disclosure. In some embodiments, the kit comprises a reagent (e.g., one or more antibodies of the present disclosure) for detecting the wild-type counterparts of a polypeptide encoded by a gene comprising one or more alterations of the disclosure. In some embodiments, the reagent comprises one or more antibodies of the present disclosure capable of binding to a polypeptide encoded by a gene comprising one or more alterations of the disclosure, or to wild-type counterparts of the polypeptide. In some embodiments, the reagent comprises one or more antibodies of the present disclosure capable of distinguishing a polypeptide encoded by a gene comprising one or more alterations of the disclosure from wild-type counterparts of the polypeptide. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), or hyperdiffusion chromatography). In some embodiments, the kit further comprises instructions for detecting a polypeptide encoded by a gene comprising one or more alterations of the disclosure, e.g., using one or more antibodies of the present disclosure.

Also provided herein are kits for detecting one or more mutations in one or more genes selected from CDKN2A, CDKN2B, VHL, PBRM1, SETD2, BAP1, TP53, PTEN, TERT, TSC1, PIK3CA, ATM, NF2, or MET, in a sample from an individual having a cancer, e.g., a ccRCC. In some embodiments, a kit provided herein comprises a reagent (e.g., one or more oligonucleotides, primers, probes or baits of the present disclosure) for detecting one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the kit comprises a reagent (e.g., one or more oligonucleotides, primers, probes or baits of the present disclosure) for detecting a wild-type counterpart of one or more genes selected from CDKN2A, CDKN2B, VHL, PBRM1, SETD2, BAP1, TP53, PTEN, TERT, TSC1, PIK3CA, ATM, NF2, or MET. In some embodiments, the reagent comprises one or more oligonucleotides, primers, probes or baits of the present disclosure capable of hybridizing to a nucleic acid molecule comprising or encoding one or more mutations in one or more genes selected from CDKN2A, CDKN2B, VHL, PBRM1, SETD2, BAP1, TP53, PTEN, TERT, TSC1, PIK3CA, ATM, NF2, or MET, or to a wild-type counterpart. In some embodiments, the reagent comprises one or more oligonucleotides, primers, probes or baits of the present disclosure capable of distinguishing a nucleic acid molecule comprising or encoding one or more mutations in one or more genes selected from CDKN2A, CDKN2B, VHL, PBRM1, SETD2, BAP1, TP53, PTEN, TERT, TSC1, PIK3CA, ATM, NF2, or MET, from a corresponding wild-type counterpart of the nucleic acid molecule. In some embodiments, the kit is for use according to any method of detecting or measuring one or more gene alterations in nucleic acid molecules known in the art or described herein, such as sequencing, exome sequencing, whole genome sequencing, gene-targeted sequencing, next-generation sequencing, PCR, in situ hybridization methods, a nucleic acid hybridization assay, an amplification-based assay, a PCR-RFLP assay, real-time PCR, sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting one or more mutations in one or more genes selected from CDKN2A, CDKN2B, VHL, PBRM1, SETD2, BAP1, TP53, PTEN, TERT, TSC1, PIK3CA, ATM, NF2, or MET, e.g., using one or more oligonucleotides, primers, probes or baits of the present disclosure.

Also provided herein are kits for detecting a polypeptide of the disclosure, e.g., a polypeptide encoded by a gene comprising one or more alterations (e.g., in one or more genes selected from CDKN2A, CDKN2B, VHL, PBRM1, SETD2, BAP1, TP53, PTEN, TERT, TSC1, PIK3CA, ATM, NF2, or MET). In some embodiments, a kit provided herein comprises a reagent (e.g., one or more antibodies of the present disclosure) for detecting a polypeptide encoded by a gene comprising one or more alterations of the disclosure, e.g., one or more mutations in one or more genes selected from CDKN2A, CDKN2B, VHL, PBRM1, SETD2, BAP1, TP53, PTEN, TERT, TSC1, PIK3CA, ATM, NF2, or MET. In some embodiments, the kit comprises a reagent (e.g., one or more antibodies of the present disclosure) for detecting the wild-type counterparts of a polypeptide encoded by a gene, e.g., one or more genes selected from CDKN2A, CDKN2B, VHL, PBRM1, SETD2, BAP1, TP53, PTEN, TERT, TSC1, PIK3CA, ATM, NF2, or MET, comprising one or more alterations of the disclosure. In some embodiments, the reagent comprises one or more antibodies of the present disclosure capable of binding to a polypeptide encoded by a gene, e.g., one or more genes selected from CDKN2A, CDKN2B, VHL, PBRM1, SETD2, BAP1, TP53, PTEN, TERT, TSC1, PIK3CA, ATM, NF2, or MET, comprising one or more alterations of the disclosure or to wild-type counterparts of the polypeptide. In some embodiments, the reagent comprises one or more antibodies of the present disclosure capable of distinguishing a polypeptide encoded by a gene, e.g., one or more genes selected from CDKN2A, CDKN2B, VHL, PBRM1, SETD2, BAP1, TP53, PTEN, TERT, TSC1, PIK3CA, ATM, NF2, or MET, comprising one or more alterations of the disclosure from wild-type counterparts of the polypeptide. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a polypeptide encoded by a gene, e.g., one or more genes selected from CDKN2A, CDKN2B, VHL, PBRM1, SETD2, BAP1, TP53, PTEN, TERT, TSC1, PIK3CA, ATM, NF2, or MET, comprising one or more alterations of the disclosure, e.g., using one or more antibodies of the present disclosure.

### Expression Vectors, Host Cells and Recombinant Cells

Provided herein are vectors comprising a nucleic acid molecule, a bait, a probe, or an oligonucleotide described herein, or fragments thereof.

In some embodiments, a vector provided herein comprises a nucleic acid molecule comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703); or a nucleic acid molecule encoding a polypeptide encoded by a gene comprising one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703).

In some embodiments, a vector provided herein is a nucleic acid molecule capable of transporting another nucleic acid to which it has been linked (e.g., nucleic acid molecules comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising a nucleic acid molecule comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), baits, probes, or oligonucleotides described herein, or fragments thereof) are also contemplated herein, including, e.g., replication defective retroviruses, adenoviruses and adeno-associated viruses.

In some embodiments, a vector provided herein comprises a nucleic acid molecule comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), a bait, a probe, or an oligonucleotide of the disclosure in a form suitable for expression thereof in a host cell. In some embodiments, the vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., nucleic acid molecules comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), baits, probes, or oligonucleotides described herein, or fragments thereof). In some embodiments, a regulatory sequence directs tissue-specific expression of a nucleotide sequence (e.g., nucleic acid molecules comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), baits, probes, or oligonucleotides described herein, or fragments thereof). In some embodiments, a regulatory sequence directs inducible expression of a nucleotide sequence (e.g., nucleic acid molecules comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises a nucleic acid molecule comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), a bait, a probe, or an oligonucleotide of the disclosure in the sense or the anti-sense orientation. In some embodiments, a vector (e.g., an expression vector) provided herein is introduced into host cells to thereby produce a polypeptide, e.g., a polypeptide encoded by a nucleic acid comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), or a fragment or mutant form thereof.

In some embodiments, the design of a vector provided herein depends on such factors as the choice of the host cell to be transformed, the level of expression desired, and the like. In some embodiments, expression vectors are designed for the expression of nucleic acid molecules comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises a nucleic acid molecule comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), wherein the nucleotide sequence of the nucleic acid molecule has been altered (e.g., codon optimized) so that the individual codons for each encoded amino acid are those preferentially utilized in the host cell.

Also provided herein are host cells, e.g., comprising nucleic acid molecules comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), polypeptides encoded by such nucleic acid molecules, baits, probes, vectors, or oligonucleotides of the disclosure. In some embodiments, a host cell (e.g., a recombinant host cell or recombinant cell) comprises a vector described herein (e.g., an expression vector described herein). In some embodiments, a nucleic acid molecule comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), bait, probe, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

Nucleic acid molecules comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), baits, probes, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing a polypeptide encoded by a nucleic acid comprising or encoding one or more gene alterations of the disclosure (e.g., a deletion of an MTAP gene, CDKN2A gene, or CDKN2B gene, or portions thereof, or one or more mutations in one or more genes selected from AKT2, ALK, APC, ARID1A, ATM, BAP1, BCL2L1, BRAF, BRCA1, BRCA2, CCND1, CCNE1, CDH1, CDKN1A, CDKN2A, CDKN2B, CHEK2, CREBBP, DNMT3A, EGFR, EP300, EPHA3, ERBB2, ERBB3, FBXW7, FGF10, FGF19, FGF3, FGF4, FGFR1, FGFR3, HRAS, KDM6A, KEAP1, KMT2D, KRAS, MCL1, MDM2, MET, MTAP, MUTYH, MYC, MYCL, NF1, NF2, NFE2L2, NOTCH3, NSD3, NTRK1, PBRM1, PIK3CA, PTEN, RAD21, RAF1, RB1, RBM10, RICTOR, ROS1, SETD2, SF3B1, SMARCA4, STAG2, STK11, TERT, TET2, TP53, TSC1, VHL, or ZNF703), e.g., by culturing a host cell described herein (e.g., into which a recombinant expression vector encoding a polypeptide has been introduced) in a suitable medium such that the polypeptide is produced. In another embodiment, the method further includes isolating the polypeptide from the medium or the host cell.

### EXEMPLARY EMBODIMENTS

The following exemplary embodiments are representative of some aspects of the invention:

Exemplary Embodiment 1: A method of identifying an individual having a urothelial bladder cancer (UCB) who may benefit from a treatment comprising a fibroblast growth factor receptor (FGFR)-targeted therapy or a phosphatase and tensin homolog (PTEN)-targeted therapy, the method comprising detecting in a sample from the individual a deletion of a methylthioadenosine phosphorylase (MTAP) gene, or of a portion thereof, and one or more mutations in a fibroblast growth factor receptor 3 (FGFR3) gene or in a PTEN gene, wherein:
(a) the presence of the deletion of the MTAP gene, or of the portion thereof, and one or more mutations in the FGFR3 gene in the sample identifies the individual as one who may benefit from a treatment comprising an FGFR-targeted therapy; or
(b) the presence of the deletion of the MTAP gene, or of the portion thereof, and one or more mutations in the PTEN gene in the sample identifies the individual as one who may benefit from a treatment comprising a PTEN-targeted therapy.

Exemplary Embodiment 2: A method of selecting a treatment for an individual having a UCB, the method comprising detecting a deletion of an MTAP gene, or of a portion thereof, and one or more mutations in an FGFR3 gene or in a PTEN gene in a sample from the individual, wherein:
(a) the presence of the deletion of the MTAP gene, or of the portion thereof, and one or more mutations in the FGFR3 gene in the sample identifies the individual as one who may benefit from a treatment comprising an FGFR-targeted therapy; or
(b) the presence of the deletion of the MTAP gene, or of the portion thereof, and one or more mutations in the PTEN gene in the sample identifies the individual as one who may benefit from a treatment comprising a PTEN-targeted therapy.

Exemplary Embodiment 3: The method of embodiment 1 or embodiment 2, wherein the presence in the sample of the deletion of the MTAP gene, or of the portion thereof, and the one or more mutations in the FGFR3 gene or in the PTEN gene identifies the individual as one who may benefit from an additional treatment comprising a protein arginine methyltransferase 5 (PRMT5)-targeted therapy.

Exemplary Embodiment 4: A method of identifying one or more treatment options for an individual having a UCB, the method comprising:
(a) detecting a deletion of an MTAP gene, or of a portion thereof, and one or more mutations in an FGFR3 gene or in a PTEN gene in a sample from the individual; and
(b) generating a report, wherein the report comprises:
(i) one or more treatment options identified for the individual based at least in part on the presence of the deletion of the MTAP gene, or of the portion thereof, and one or more mutations in the FGFR3 gene in the sample, wherein the one or more treatment options comprise an FGFR-targeted therapy; or
(ii) one or more treatment options identified for the individual based at least in part on the presence of the deletion of the MTAP gene, or of the portion thereof, and one or more mutations in the PTEN gene in the sample, wherein the one or more treatment options comprise a PTEN-targeted therapy.

Exemplary Embodiment 5: A method of identifying one or more treatment options for an individual having a UCB, the method comprising acquiring knowledge of a deletion of an MTAP gene, or of a portion thereof, and one or more mutations in an FGFR3 gene or in a PTEN gene in a sample from the individual; and generating a report comprising:
(a) one or more treatment options identified for the individual based at least in part on the knowledge of a deletion of the MTAP gene, or of a portion thereof, and one or more mutations in the FGFR3 gene in the sample, wherein the one or more treatment options comprise an FGFR-targeted therapy; or
(b) one or more treatment options identified for the individual based at least in part on the knowledge of a deletion of the MTAP gene, or of a portion thereof, and one or more mutations in the PTEN gene in the sample, wherein the one or more treatment options comprise a PTEN-targeted therapy.

Exemplary Embodiment 6: The method of embodiment 4 or embodiment 5, wherein the one or more treatment options identified for the individual further comprise a PRMT5-targeted therapy.

Exemplary Embodiment 7: A method of selecting a treatment for an individual having a UCB, comprising acquiring knowledge of a deletion of an MTAP gene, or of a portion thereof, and one or more mutations in an FGFR3 gene or in a PTEN gene in a sample from the individual, wherein:
(a) responsive to the acquisition of knowledge of a deletion of an MTAP gene, or of a portion thereof, and one or more mutations in an FGFR3 gene in the sample: (i) the individual is classified as a candidate to receive a treatment comprising an FGFR-targeted therapy; and/or (ii) the individual is identified as likely to respond to a treatment that comprises an FGFR-targeted therapy; or
(b) responsive to the acquisition of knowledge of a deletion of an MTAP gene, or of a portion thereof, and one or more mutations in a PTEN gene in the sample: (i) the individual is classified as a candidate to receive a treatment comprising a PTEN-targeted therapy; and/or (ii) the individual is identified as likely to respond to a treatment that comprises a PTEN-targeted therapy.

Exemplary Embodiment 8: The method of embodiment 7, wherein the treatment further comprises a PRMT5-targeted therapy.

Exemplary Embodiment 9: A method of predicting survival of an individual having a UCB treated with a treatment comprising an FGFR-targeted therapy or a PTEN-targeted therapy, the method comprising acquiring knowledge of a deletion of an MTAP gene, or of a portion thereof, and one or more mutations in an FGFR3 gene or in a PTEN gene in a sample from the individual, wherein responsive to the acquisition of said knowledge, the individual is predicted to have longer survival after treatment with the FGFR-targeted therapy or the PTEN-targeted therapy, as compared to an individual whose UCB does not exhibit a deletion of an MTAP gene, or of a portion thereof, and one or more mutations in an FGFR3 gene or in a PTEN gene.

Exemplary Embodiment 10: A method of treating or delaying progression of a UCB, comprising:
(a) acquiring knowledge of a deletion of an MTAP gene, or of a portion thereof, and one or more mutations in an FGFR3 gene in a sample from an individual, and responsive to said knowledge, administering to the individual an effective amount of a treatment comprising an FGFR-targeted therapy; or
(b) acquiring knowledge of a deletion of an MTAP gene, or of a portion thereof, and one or more mutations in a PTEN gene in a sample from an individual, and responsive to said knowledge, administering to the individual an effective amount of a treatment comprising a PTEN-targeted therapy.

Exemplary Embodiment 11: A method of treating or delaying progression of a UCB, comprising:
(a) responsive to acquiring knowledge of a deletion of an MTAP gene, or of a portion thereof, and one or more mutations in an FGFR3 gene in a sample from an individual, administering to the individual an effective amount of a treatment comprising an FGFR-targeted therapy; or
(b) responsive to acquiring knowledge of a deletion of an MTAP gene, or of a portion thereof, and one or more mutations in a PTEN gene in a sample from an individual, administering to the individual an effective amount of a treatment comprising a PTEN-targeted therapy.

Exemplary Embodiment 12: The method of any one of embodiments 5-11, wherein the acquiring knowledge of the deletion of the MTAP gene, or of the portion thereof, and one or more mutations in the FGFR3 gene or in the PTEN gene comprises detecting the deletion of the MTAP gene, or of the portion thereof, and the one or more mutations in the FGFR3 gene or in the PTEN gene in the sample.

Exemplary Embodiment 13: A method of treating or delaying progression of a UCB, comprising:
(a) detecting a deletion of an MTAP gene, or of a portion thereof, and one or more mutations in an FGFR3 gene in a sample from an individual, and administering to the individual an effective amount of a treatment comprising an FGFR-targeted therapy; or
(b) detecting a deletion of an MTAP gene, or of a portion thereof, and one or more mutations in a PTEN gene in a sample from an individual, and administering to the individual an effective amount of a treatment comprising a PTEN-targeted therapy.

Exemplary Embodiment 14: A method of detecting the presence or absence of a UCB in an individual, comprising:
(a) detecting the presence or absence of a UCB in a sample from an individual; and
(i) detecting the presence or absence of a deletion of an MTAP gene, or of a portion thereof, and one or more mutations in an FGFR3 gene in the sample, or
(ii) detecting the presence or absence of a deletion of an MTAP gene, or of a portion thereof, and one or more mutations in a PTEN gene in the sample.

Exemplary Embodiment 15: A method of assessing a UCB in an individual, the method comprising:
(a) detecting a deletion of an MTAP gene, or of a portion thereof, and one or more mutations in an FGFR3 gene or in a PTEN gene in a sample from the individual; and
(b) providing an assessment of the deletion of the MTAP gene, or of the portion thereof, and the one or more mutations in the FGFR3 gene or in the PTEN gene.

Exemplary Embodiment 16: A method of detecting a deletion of an MTAP gene, or of a portion thereof, and one or more mutations in an FGFR3 gene or in a PTEN gene, the method comprising detecting the deletion of the MTAP gene, or of the portion thereof, and the one or more mutations in the FGFR3 gene or in the PTEN gene in a sample from an individual having a UCB.

Exemplary Embodiment 17: A method of detecting a deletion of an MTAP gene, or of a portion thereof, and one or more mutations in an FGFR3 gene or in a PTEN gene, the method comprising:
(a) providing a sample from an individual having a UCB, wherein the sample comprises one or more nucleic acids;
(b) preparing a nucleic acid sequencing library from the one or more nucleic acids in the sample;
(c) amplifying said library using a polymerase chain reaction (PCR);
(d) selectively enriching for one or more nucleic acids comprising MTAP, FGFR3, or PTEN gene nucleotide sequences in said library to produce an enriched sample;
(e) sequencing the enriched sample, thereby producing a plurality of sequencing reads;
(f) analyzing the plurality of sequencing reads for the presence of a deletion of an MTAP gene, or of a portion thereof, and one or more mutations in an FGFR3 gene or in a PTEN gene;
(g) detecting, based on the analyzing step, a deletion of an MTAP gene, or of a portion thereof, and one or more mutations in an FGFR3 gene or in a PTEN gene in the sample from the individual.

Exemplary Embodiment 18: The method of any one of embodiments 1-4 and 12-16, further comprising selectively enriching for one or more of: (a) one or more nucleic acids comprising nucleotide sequences that comprise the deletion of the MTAP gene, or of the portion thereof, (b) one or more nucleic acids comprising nucleotide sequences that comprise the one or more mutations in the FGFR3 gene, or (c) one or more nucleic acids comprising nucleotide sequences that comprise the one or more mutations in the PTEN gene; wherein the selectively enriching produces an enriched sample.

Exemplary Embodiment 19: A method of treating or delaying progression of a UCB, comprising:
(a) administering to an individual having a UCB an effective amount of a treatment comprising an FGFR-targeted therapy, wherein the UCB comprises a deletion of an MTAP gene, or of a portion thereof, and one or more mutations in an FGFR3 gene; or
(b) administering to an individual having a UCB an effective amount of a treatment comprising a PTEN-targeted therapy, wherein the UCB comprises a deletion of an MTAP gene, or of a portion thereof, and one or more mutations in a PTEN gene.

Exemplary Embodiment 20: The method of any one of embodiments 1-18, further comprising acquiring knowledge of or detecting in the sample a deletion of a CDKN2A gene, or of a portion thereof; and/or a deletion of a CDKN2B gene, or of a portion thereof.

Exemplary Embodiment 21: The method of any one of embodiments 1-18 and 20, comprising acquiring knowledge of or detecting in the sample one or more mutations in one or more genes, wherein the one or more genes comprise TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1.

Exemplary Embodiment 22: The method of any one of embodiments 1-21, wherein the individual has one or more mutations in one or more genes, wherein the one or more genes comprise TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1.

Exemplary Embodiment 23: The method of embodiment 21 or embodiment 22, further comprising administering to the individual an effective amount of a treatment comprising an anti-cancer therapy.

Exemplary Embodiment 24: The method of any one of embodiments 21-23, further comprising selectively enriching for one or more nucleic acids comprising nucleotide sequences that comprise one or more mutations in one or more genes, wherein the one or more genes comprise TERT, TP53, FGFR3, KDM6A, PIK3CA, KMT2D, CCND1, FGF19, FGF4, FGF3, ARID1A, TSC1, ERBB2, STAG2, MDM2, CREBBP, EP300, CDKN1A, ATM, PTEN, NFE2L2, KRAS, MCL1, FBXW7, MYC, ERBB3, RBM10, SMARCA4, HRAS, TET2, MYCL, RAF1, NF1, EGFR, NSD3, RICTOR, ZNF703, DNMT3A, NOTCH3, FGFR1, CHEK2, APC, AKT2, MUTYH, EPHA3, BRCA2, BRAF, or BAP1, to produce an enriched sample.

Exemplary Embodiment 25: The method of any one of embodiments 21-24, wherein the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 26: The method of any one of embodiments 1-25, wherein the individual has a deletion of a CDKN2A gene, or of a portion thereof; and/or a deletion of a CDKN2B gene, or of a portion thereof.

Exemplary Embodiment 27: The method of embodiment 26, wherein the individual has a heterozygous or a homozygous deletion of a CDKN2A gene, or of a portion thereof; and/or a heterozygous or a homozygous deletion of a CDKN2B gene, or of a portion thereof.

Exemplary Embodiment 28: The method of any one of embodiments 1-27, wherein the individual has a heterozygous or a homozygous deletion of the MTAP gene, or of a portion thereof.

Exemplary Embodiment 29: The method of any one of embodiments 10-13 and 18-28, further comprising administering to the individual a PRMT5-targeted therapy.

Exemplary Embodiment 30: The method of any one of embodiments 1-29, wherein the individual is being treated with a PRMT5-targeted therapy.

Exemplary Embodiment 31: The method of any one of embodiments 3, 6, 8, and 29-30, wherein the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

Exemplary Embodiment 32: The method of any one of embodiments 1-31, wherein the one or more mutations in the FGFR3 gene or the PTEN gene comprise one or more of an insertion, deletion or substitution of one or more nucleotides, a genomic rearrangement, an alteration in a promoter, a gene fusion, or a copy number alteration.

Exemplary Embodiment 33: The method of any one of embodiments 1-32, wherein the one or more mutations in the FGFR3 gene or the PTEN gene result in one or more of an insertion, deletion or substitution of one or more amino acid residues in a polypeptide encoded by the FGFR3 gene or the PTEN gene.

Exemplary Embodiment 34: The method of any one of embodiments 1-13 and 18-33, wherein the FGFR-targeted therapy or the PTEN-targeted therapy is a small molecule inhibitor, an antibody, a cellular therapy, or a nucleic acid.

Exemplary Embodiment 35: The method of embodiment 34, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 36: The method of embodiment 34, wherein the nucleic acid comprises a double-stranded RNA (dsRNA), a small interfering RNA (siRNA), or a small hairpin RNA (shRNA).

Exemplary Embodiment 37: The method of embodiment 34, wherein the FGFR-targeted therapy comprises one or more of: a multi-kinase inhibitor, an FGFR-selective inhibitor, an FGFR3-specific inhibitor, or a combination therapy.

Exemplary Embodiment 38: The method of embodiment 37, wherein the multi-kinase inhibitor comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), or orantinib (TSU-68).

Exemplary Embodiment 39: The method of embodiment 37, wherein the FGFR-selective inhibitor comprises one or more of PRN1371, XL-228, AZ12908010 (AZ8010), Debio-1347 (CH5183284), FIIN-2, LY2874455, Infigratinib (BGJ398, NVP-BGJ398), Pemigatinib (Pemazyre®, INCB054828), Erdafitinib (JNJ-42756493, Balversa®), ASP5878, TAS-120, PRN1371, or PKC412.

Exemplary Embodiment 40: The method of embodiment 37, wherein the FGFR3-specific inhibitor comprises one or more of Vofatamab or MFGR1877S.

Exemplary Embodiment 41: The method of embodiment 37, wherein the combination therapy comprises one or more of: an FGFR-targeted therapy and a PD-1 or a PD-L1-targeted therapy; an FGFR-targeted therapy and an EGFR inhibitor; an FGFR-targeted therapy and an immunotherapy; an FGFR-targeted therapy and a MAPK inhibitor; an FGFR-targeted therapy and a PI3K inhibitor; an FGFR-targeted therapy and an AKT inhibitor; or an FGFR-targeted therapy and a VEGF inhibitor.

Exemplary Embodiment 42: The method of embodiment 34, wherein the PTEN-targeted therapy comprises one or more of: a PI3K inhibitor, an AKT inhibitor, an mTOR inhibitor, or a MET inhibitor.

Exemplary Embodiment 43: The method of any one of embodiments 1-4, 12-18, and 20-42, wherein the deletion of the MTAP gene or of the portion thereof, or the one or more mutations in the FGFR3 gene or the PTEN gene are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 44: The method of any one of embodiments 1-4, 12-18, and 20-42, wherein the deletion of the MTAP gene or of the portion thereof, or the one or more mutations in the FGFR3 gene or the PTEN gene are detected in a polypeptide encoded by the MTAP gene, the FGFR3 gene, or the PTEN gene.

Exemplary Embodiment 45: The method of embodiment 44, wherein the deletion of the MTAP gene or of the portion thereof, or the one or more mutations in the FGFR3 gene or the PTEN gene are detected in the sample by one or more of: immunoblotting, enzyme linked immunosorbent assay (ELISA), immunohistochemistry, or mass spectrometry.

Exemplary Embodiment 46: The method of any one of embodiments 20-45, wherein the deletion of the CDKN2A gene or of the portion thereof, and/or the deletion of the CDKN2B gene or of the portion thereof, are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 47: The method of any one of embodiments 20-45, wherein the deletion of the CDKN2A gene or of the portion thereof, and/or the deletion of the CDKN2B gene or of the portion thereof, are detected in a polypeptide encoded by the CDKN2A gene or the CDKN2B gene.

Exemplary Embodiment 48: The method of embodiment 47, wherein the deletion of the CDKN2A gene or of the portion thereof, and/or the deletion of the CDKN2B gene or of the portion thereof, are detected in the sample by one or more of: immunoblotting, enzyme linked immunosorbent assay (ELISA), immunohistochemistry, or mass spectrometry.

Exemplary Embodiment 49: A method of identifying an individual having a urothelial bladder cancer (UCB) who may benefit from a treatment comprising an immunotherapy or a retinoblastoma (RB1)-targeted therapy, the method comprising detecting in a sample from the individual one or more of: (a) a tumor mutational burden (TMB) of at least about 10 mut/Mb or at least about 20 mut/Mb, (b) a PD-L1-positive UCB, or (c) one or more mutations in an RB1 gene; wherein:
(i) the presence in the sample of a TMB of at least about 10 mut/Mb or at least about 20 mut/Mb, or of a PD-L1-positive UCB identifies the individual as one who may benefit from a treatment comprising an immunotherapy; or
(ii) the presence in the sample of one or more mutations in the RB1 gene identifies the individual as one who may benefit from a treatment comprising an RB1-targeted therapy.

Exemplary Embodiment 50: A method of selecting a treatment for an individual having a UCB, the method comprising detecting one or more of: (a) a TMB of at least about 10 mut/Mb or at least about 20 mut/Mb, (b) a PD-L1-positive UCB, or (c) one or more mutations in an RB1 gene, in a sample from the individual; wherein:
(i) the presence in the sample of a TMB of at least about 10 mut/Mb or at least about 20 mut/Mb, or of a PD-L1-positive UCB identifies the individual as one who may benefit from a treatment comprising an immunotherapy; or
(ii) the presence in the sample of one or more mutations in the RB1 gene identifies the individual as one who may benefit from a treatment comprising an RB1-targeted therapy.

Exemplary Embodiment 51: A method of identifying one or more treatment options for an individual having a UCB, the method comprising:
(a) detecting in a sample from the individual one or more of: (i) a TMB of at least about 10 mut/Mb or at least about 20 mut/Mb, (ii) a PD-L1-positive UCB, or (iii) one or more mutations in an RB1 gene; and
(b) generating a report, wherein the report comprises:
(i) one or more treatment options identified for the individual based at least in part on the presence of a TMB of at least about 10 mut/Mb or at least about 20 mut/Mb, or of a PD-L1-positive UCB in the sample, wherein the one or more treatment options comprise an immunotherapy; or
(ii) one or more treatment options identified for the individual based at least in part on the presence of one or more mutations in the RB1 gene in the sample, wherein the one or more treatment options comprise an RB1-targeted therapy.

Exemplary Embodiment 52: A method of identifying one or more treatment options for an individual having a UCB, the method comprising acquiring knowledge of one or more of: (a) a TMB of at least about 10 mut/Mb or at least about 20 mut/Mb, (b) a PD-L1-positive UCB, or (c) one or more mutations in an RB1 gene, in a sample from the individual; and generating a report comprising: (i) one or more treatment options identified for the individual based at least in part on the knowledge of a TMB of at least about 10 mut/Mb or at least about 20 mut/Mb, or of a PD-L1-positive UCB in the sample, wherein the one or more treatment options comprise an immunotherapy; or
(ii) one or more treatment options identified for the individual based at least in part on the knowledge of one or more mutations in the RB1 gene in the sample, wherein the one or more treatment options comprise an RB1-targeted therapy.

Exemplary Embodiment 53: A method of selecting a treatment for an individual having a UCB, comprising acquiring knowledge of one or more of: (a) a TMB of at least about 10 mut/Mb or at least about 20 mut/Mb, (b) a PD-L1-positive UCB, or (c) one or more mutations in an RB1 gene, in a sample from the individual, wherein:
(a) responsive to acquisition of knowledge of a TMB of at least about 10 mut/Mb or at least about 20 mut/Mb, or of a PD-L1-positive UCB in the sample: (i) the individual is classified as a candidate to receive a treatment comprising an immunotherapy; and/or (ii) the individual is identified as likely to respond to a treatment that comprises an immunotherapy; or
(b) responsive to acquisition of knowledge of one or more mutations in the RB1 gene in the sample: (i) the individual is classified as a candidate to receive a treatment comprising an RB1-targeted therapy; and/or (ii) the individual is identified as likely to respond to a treatment that comprises an RB1-targeted therapy.

Exemplary Embodiment 54: A method of predicting survival of an individual having UCB treated with a treatment comprising an immunotherapy or an RB1-targeted therapy, the method comprising acquiring knowledge of one or more of: (a) a TMB of at least about 10 mut/Mb or at least about 20 mut/Mb, (b) a PD-L1-positive UCB, or (c) one or more mutations in an RB1 gene, in a sample from the individual; wherein responsive to acquisition of said knowledge, the individual is predicted to have longer survival after treatment with the immunotherapy or the RB1-targeted therapy, as compared to an individual whose UCB does not exhibit a TMB of at least about 10 mut/Mb or at least about 20 mut/Mb, a PD-L1-positive UCB, or one or more mutations in the RB1 gene.

Exemplary Embodiment 55: A method of treating or delaying progression of a UCB, comprising:
(a) acquiring knowledge of a TMB of at least about 10 mut/Mb or at least about 20 mut/Mb, or a PD-L1-positive UCB in a sample from an individual, and responsive to said knowledge, administering to the individual an effective amount of a treatment comprising an immunotherapy; or
(b) acquiring knowledge of one or more mutations in an RB1 gene in a sample from an individual, and responsive to said knowledge, administering to the individual an effective amount of a treatment comprising an RB1-targeted therapy.

Exemplary Embodiment 56: A method of treating or delaying progression of a UCB, comprising:
(a) responsive to acquiring knowledge of a TMB of at least about 10 mut/Mb or at least about 20 mut/Mb, or a PD-L1-positive UCB in a sample from an individual, administering to the individual an effective amount of a treatment comprising an immunotherapy; or
(b) responsive to acquiring knowledge of one or more mutations in an RB1 gene in a sample from an individual, administering to the individual an effective amount of a treatment comprising an RB1-targeted therapy.

Exemplary Embodiment 57: The method of any one of embodiments 52-56, wherein acquiring knowledge of a TMB of at least about 10 mut/Mb or at least about 20 mut/Mb, a PD-L1-positive UCB, or one or more mutations in an RB1 gene comprises detecting the TMB of at least about 10 mut/Mb or at least about 20 mut/Mb, the PD-L1-positive UCB, or the one or more mutations in the RB1 gene in the sample.

Exemplary Embodiment 58: A method of treating or delaying progression of a UCB, comprising:
(a) detecting a TMB of at least about 10 mut/Mb or at least about 20 mut/Mb, or a PD-L1-positive UCB in a sample from an individual, and administering to the individual an effective amount of a treatment comprising an immunotherapy; or
(b) detecting one or more mutations in an RB1 gene in a sample from an individual, and administering to the individual an effective amount of a treatment comprising an RB1-targeted therapy.

Exemplary Embodiment 59: A method of detecting the presence or absence of a UCB in an individual, comprising:
(a) detecting the presence or absence of a UCB in a sample from an individual; and
(i) detecting the presence or absence of a TMB of at least about 10 mut/Mb or at least about 20 mut/Mb, or a PD-L1-positive UCB in the sample, or
(ii) detecting the presence or absence of one or more mutations in an RB1 gene in the sample.

Exemplary Embodiment 60: A method of assessing a UCB in an individual, the method comprising:
(a) detecting one or more of: (i) a TMB of at least about 10 mut/Mb or at least about 20 mut/Mb, (ii) a PD-L1-positive UCB, or (iii) one or more mutations in an RB1 gene, in a sample from the individual; and
(b) providing an assessment of the TMB of at least about 10 mut/Mb or at least about 20 mut/Mb, the PD-L1-positive UCB, or the one or more mutations in the RB1 gene in the sample.

Exemplary Embodiment 61: A method of detecting a TMB of at least about 10 mut/Mb or at least about 20 mut/Mb, a PD-L1-positive UCB, or one or more mutations in an RB1 gene, the method comprising detecting the TMB of at least about 10 mut/Mb or at least about 20 mut/Mb, the PD-L1-positive UCB, or the one or more mutations in the RB1 gene in a sample from an individual having a UCB.

Exemplary Embodiment 62: A method of detecting one or more mutations in an RB1 gene, the method comprising:
(a) providing a sample from an individual having a UCB, wherein the sample comprises one or more nucleic acids;
(b) preparing a nucleic acid sequencing library from the one or more nucleic acids in the sample; (c) amplifying said library using a polymerase chain reaction (PCR);
(d) selectively enriching for one or more nucleic acids comprising RB1 gene nucleotide sequences in said library to produce an enriched sample;
(e) sequencing the enriched sample, thereby producing a plurality of sequencing reads;
(f) analyzing the plurality of sequencing reads for the presence of one or more mutations in an RB1 gene;
(g) detecting, based on the analyzing step, one or more mutations in an RB1 gene in the sample from the individual.

Exemplary Embodiment 63: A method of detecting a TMB of at least about 10 mut/Mb or at least about 20 mut/Mb, the method comprising:
(a) providing a sample from an individual having a UCB, wherein the sample comprises one or more nucleic acids;
(b) preparing a nucleic acid sequencing library from the one or more nucleic acids in the sample;
(c) amplifying said library using a polymerase chain reaction (PCR);
(d) sequencing said library, thereby producing a plurality of sequencing reads;
(e) analyzing the plurality of sequencing reads for the presence of a TMB of at least about 10 mut/Mb or at least about 20 mut/Mb;
(f) detecting, based on the analyzing step, a TMB of at least about 10 mut/Mb or at least about 20 mut/Mb in the sample from the individual.

Exemplary Embodiment 64: A method of treating or delaying progression of a UCB, comprising:
(a) administering to an individual having a UCB an effective amount of a treatment comprising an immunotherapy, wherein the UCB comprises a TMB of at least about 10 mut/Mb or at least about 20 mut/Mb, or wherein the UCB is PD-L1-positive; or
(b) administering to an individual having a UCB an effective amount of a treatment comprising an RB1-targeted therapy, wherein the UCB comprises one or more mutations in an RB1 gene.

Exemplary Embodiment 65: The method of any one of embodiments 52-57, further comprising acquiring knowledge of the absence of a deletion of an MTAP gene, or of a portion thereof, in the sample.

Exemplary Embodiment 66: The method of embodiment 65, wherein acquiring knowledge of the absence of a deletion of an MTAP gene, or of a portion thereof, comprises detecting the absence of a deletion of an MTAP gene, or of a portion thereof, in the sample.

Exemplary Embodiment 67: The method of any one of embodiments 49-51, 57-63, and 65-66, further comprising detecting the absence of a deletion of an MTAP gene, or of a portion thereof, in the sample.

Exemplary Embodiment 68: The method of any one of embodiments 49-63 and 65-67, comprising acquiring knowledge of or detecting in the sample one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2.

Exemplary Embodiment 69: The method of any one of embodiments 49-68, wherein the individual has one or more mutations in one or more genes, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2.

Exemplary Embodiment 70: The method of embodiment 68 or embodiment 69, further comprising administering to the individual an effective amount of a treatment comprising anti-cancer therapy.

Exemplary Embodiment 71: The method of embodiment 68 or embodiment 69, comprising selectively enriching for one or more nucleic acids comprising nucleotide sequences that comprise one or more mutations in one or more genes to produce an enriched sample, wherein the one or more genes comprise TERT, TP53, ARID1A, KMT2D, KDM6A, PIK3CA, ERBB2, CDKN2A, FGFR3, CCND1, RAF1, CREBBP, FGF19, CDKN2B, STAG2, CDKN1A, MDM2, FGF3, FGF4, CCNE1, EP300, MCL1, ERBB3, TSC1, RICTOR, ZNF703, FBXW7, RBM10, NSD3, KRAS, CDH1, ATM, FGF10, RAD21, PTEN, MYCL, EGFR, BCL2L1, AKT2, TET2, FGFR1, DNMT3A, MYC, EPHA3, APC, SMARCA4, SF3B1, PBRM1, CHEK2, or BRCA2.

Exemplary Embodiment 72: The method of any one of embodiments 68-71, wherein the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 73: The method of embodiment 68 or embodiment 69, wherein the one or more mutations in the one or more genes are detected in a polypeptide encoded by the one or more genes.

Exemplary Embodiment 74: The method of embodiment 73, wherein the one or more mutations in the one or more genes are detected in the sample by one or more of immunoblotting, enzyme linked immunosorbent assay (ELISA), immunohistochemistry, or mass spectrometry.

Exemplary Embodiment 75: The method of embodiment 66 or embodiment 67, further comprising selectively enriching for one or more nucleic acids comprising nucleotide sequences that comprise a deletion of the MTAP gene, or of a portion thereof, to produce an enriched sample.

Exemplary Embodiment 76: The method of any one of embodiments 66-75, wherein the absence of a deletion of an MTAP gene, or of a portion thereof, is detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 77: The method of any one of embodiments 49-76, wherein the individual does not have a deletion of an MTAP gene, or of a portion thereof.

Exemplary Embodiment 78: The method of any one of embodiments 49-51, 57-62, and 65-77, further comprising selectively enriching for one or more nucleic acids comprising nucleotide sequences that comprise the one or more mutations in the RB1 gene to produce an enriched sample.

Exemplary Embodiment 79: The method of any one of embodiments 49-62 and 64-78, wherein the one or more mutations in the RB1 gene comprise one or more of an insertion, deletion or substitution of one or more nucleotides, a genomic rearrangement, an alteration in a promoter of the RB1 gene, a gene fusion, or a copy number alteration.

Exemplary Embodiment 80: The method of any one of embodiments 49-62 and 64-79, wherein the one or more mutations in the RB1 gene result in one or more of an insertion, deletion or substitution of one or more amino acid residues in a polypeptide encoded by the RB1 gene.

Exemplary Embodiment 81: The method of any one of embodiments 49-51, 57-62, and 65-80, wherein the one or more mutations in the RB1 gene are detected in the sample by one or more of: an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 82: The method of any one of embodiments 49-51, 57-61, and 65-80, wherein the one or more mutations in the RB1 gene are detected in a polypeptide encoded by the RB1 gene.

Exemplary Embodiment 83: The method of embodiment 82, wherein the one or more mutations in the RB1 gene are detected in the sample by one or more of immunoblotting, enzyme linked immunosorbent assay (ELISA), immunohistochemistry, or mass spectrometry.

Exemplary Embodiment 84: The method of any one of embodiments 49-51, 57-61, 63, and 65-83, wherein detecting a TMB of at least about 10 mut/Mb or at least about 20 mut/Mb comprises measuring the level of TMB in the sample.

Exemplary Embodiment 85: The method of embodiment 84, wherein TMB is measured in the sample by whole exome sequencing, whole genome sequencing, or gene-targeted sequencing.

Exemplary Embodiment 86: The method of embodiment 85, wherein TMB is measured on about 0.80 Mb of sequenced DNA.

Exemplary Embodiment 87: The method of embodiment 85, wherein TMB is measured on between about 0.83 Mb and about 1.14 Mb of sequenced DNA.

Exemplary Embodiment 88: The method of embodiment 85, wherein TMB is measured on about 1.1 Mb of sequenced DNA.

Exemplary Embodiment 89: The method of embodiment 85, wherein TMB is measured on up to about 1.1 Mb of sequenced DNA.

Exemplary Embodiment 90: The method of any one of embodiments 49-51, 57-61, and 65-89, wherein detecting a PD-L1-positive UCB comprises measuring the level of PD-L1 expression in the sample.

Exemplary Embodiment 91: The method of embodiment 90, wherein the level of PD-L1 expression is measured using an immunohistochemistry assay.

Exemplary Embodiment 92: The method of embodiment 91, wherein the level of PD-L1 expression is determined based on PD-L1 expression on tumor cells.

Exemplary Embodiment 93: The method of embodiment 92, wherein the PD-L1-positive UCB comprises about 50% or more PD-L1-positive tumor cells, wherein the percent of PD-L1-positive tumor cells is determined in the sample.

Exemplary Embodiment 94: The method of any one of embodiments 49-58 and 64-93, wherein the immunotherapy or the RB1-targeted therapy is a small molecule inhibitor, an antibody, a cellular therapy, or a nucleic acid.

Exemplary Embodiment 95: The method of embodiment 94, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 96: The method of embodiment 94, wherein the nucleic acid comprises a double-stranded RNA (dsRNA), a small interfering RNA (siRNA), or a small hairpin RNA (shRNA).

Exemplary Embodiment 97: The method of embodiment 94, wherein the RB1-targeted therapy comprises one or more of a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor.

Exemplary Embodiment 98: The method of embodiment 94, wherein the immunotherapy comprises one or more of a checkpoint inhibitor, a cancer vaccine, a cell-based therapy, a T cell receptor (TCR)-based therapy, an adjuvant immunotherapy, a cytokine immunotherapy, or an oncolytic virus therapy.

Exemplary Embodiment 99: The method of any one of embodiments 1-98, wherein the UCB is chemorefractory UCB.

Exemplary Embodiment 100: The method of any one of embodiments 1-99, wherein the UCB is metastatic UCB.

Exemplary Embodiment 101: The method of any one of embodiments 1-13, 18-58, and 64-100, wherein the treatment or the one or more treatment options further comprise an additional anti-cancer treatment.

Exemplary Embodiment 102: The method of embodiment 101, wherein the additional anti-cancer treatment comprises one or more of a small molecule inhibitor, an antibody, a cellular therapy, or a nucleic acid.

Exemplary Embodiment 103: The method of embodiment 102, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 104: The method of embodiment 102, wherein the nucleic acid comprises a double-stranded RNA (dsRNA), a small interfering RNA (siRNA), or a small hairpin RNA (shRNA).

Exemplary Embodiment 105: The method of embodiment 101, wherein the additional anti-cancer treatment comprises one or more of a surgery, radiotherapy, chemotherapy, anti-angiogenic therapy, anti-DNA repair therapy, anti-inflammatory therapy, an anti-neoplastic agent, a chemotherapeutic agent, a growth inhibitory agent, an anti-angiogenic agent, or a cytotoxic agent.

Exemplary Embodiment 106: The method of any one of embodiments 1-18, 20-63, and 65-105, wherein the sample from the individual comprises fluid, cells, or tissue.

Exemplary Embodiment 107: The method of any one of embodiments 1-18, 20-63, and 65-106, wherein the sample from the individual comprises a tumor biopsy or a circulating tumor cell.

Exemplary Embodiment 108: The method of embodiment 106 or embodiment 107, wherein the sample from the individual comprises one or more nucleic acids.

Exemplary Embodiment 109: The method of embodiment 108, wherein the sample from the individual comprises mRNA, genomic DNA, circulating tumor DNA, cell-free DNA, or cell-free RNA.

Exemplary Embodiment 110: The method of embodiment 106 or embodiment 107, wherein the sample from the individual comprises one or more polypeptides.

Exemplary Embodiment 111: The method of any one of embodiments 1-18, 20-63, and 65-105, wherein the sample is a formalin-fixed paraffin-embedded (FFPE) sample.

Exemplary Embodiment 112: The method of any one of embodiments 1-18, 20-63, and 65-105, wherein the sample comprises one or more nucleic acids and/or one or more polypeptides obtained from an FFPE sample from the individual.

Exemplary Embodiment 113: The method of embodiment 112, wherein the one or more nucleic acids comprise mRNA and/or genomic DNA.

Exemplary Embodiment 114: The method of any one of embodiments 106-113, wherein the sample is derived from a UCB in the individual.

Exemplary Embodiment 115: The method of any one of embodiments 17-18, 24, 62, 71, 75, and 78, wherein the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample.

Exemplary Embodiment 116: The method of embodiment 115, wherein the bait comprises a capture nucleic acid molecule configured to hybridize to the one or more nucleic acids.

Exemplary Embodiment 117: The method of embodiment 116, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and 200 nucleotides.

Exemplary Embodiment 118: The method of any one of embodiments 115-117, wherein the bait is conjugated to an affinity reagent or to a detection reagent.

Exemplary Embodiment 119: The method of embodiment 118, wherein the affinity reagent is an antibody, an antibody fragment, or biotin, or wherein the detection reagent is a fluorescent marker.

Exemplary Embodiment 120: The method of any one of embodiments 116-119, wherein the capture nucleic acid molecule comprises a DNA, RNA, or mixed DNA/RNA molecule.

Exemplary Embodiment 121: The method of any one of embodiments 17-18, 24, 62, 71, 75, and 78, wherein the selectively enriching comprises amplifying the one or more nucleic acids in the sample using a polymerase chain reaction (PCR) to produce the enriched sample.

Exemplary Embodiment 122: The method of any one of embodiments 115-121, further comprising sequencing the one or more nucleic acid molecules in the enriched sample.

Exemplary Embodiment 123: A system comprising:
a memory configured to store one or more program instructions; and
one or more processors configured to execute the one or more program instructions, wherein the one or more program instructions when executed by the one or more processors are configured to:
(a) obtain a plurality of sequence reads of one or more nucleic acids, wherein the one or more nucleic acids are derived from a sample obtained from an individual having a urothelial bladder cancer (UCB);
(b) analyze the plurality of sequence reads for the presence of a deletion of an methylthioadenosine phosphorylase (MTAP) gene, or of a portion thereof, and one or more mutations in a fibroblast growth factor receptor 3 (FGFR3) gene or in a phosphatase and tensin homolog (PTEN) gene; and
(c) detect, based on the analyzing, a deletion of an MTAP gene, or of a portion thereof, and one or more mutations in an FGFR3 gene or in a PTEN gene in the sample.

Exemplary Embodiment 124: A system, comprising:
a memory configured to store one or more program instructions; and one or more processors configured to execute the one or more program instructions, the one or more program instructions when executed by the one or more processors are configured to:
(a) obtain a plurality of sequence reads of one or more nucleic acids, wherein the one or more nucleic acids are derived from a sample obtained from an individual;
(b) analyze the plurality of sequence reads for the presence of a deletion of a methylthioadenosine phosphorylase (MTAP) gene, or of a portion thereof, and one or more mutations in a fibroblast growth factor receptor 3 (FGFR3) gene or in a phosphatase and tensin homolog (PTEN) gene; and
(c) detect, based on the analyzing, a deletion of an MTAP gene, or of a portion thereof, and one or more mutations in an FGFR3 gene or in a PTEN gene in the sample.

Exemplary Embodiment 125: The system of embodiment 124, wherein the sample is obtained from an individual having a urothelial bladder cancer (UCB).

Exemplary Embodiment 126: The system of any one of embodiments 123-125, wherein the one or more mutations comprise one or more of an insertion, deletion or substitution of one or more nucleotides, a genomic rearrangement, an alteration in a promoter, a gene fusion, or a copy number alteration.

Exemplary Embodiment 127: The system of any one of embodiments 123-126, wherein the one or more mutations result in one or more of an insertion, deletion or substitution of one or more amino acid residues in a polypeptide encoded by the FGFR3 gene or the PTEN gene.

Exemplary Embodiment 128: The system of any one of embodiments 123-127, wherein the plurality of sequence reads is obtained by sequencing, whole exome sequencing, whole genome sequencing, gene-targeted sequencing, or next-generation sequencing.

Exemplary Embodiment 129: A non-transitory computer readable storage medium comprising one or more programs executable by one or more computer processors for performing a method, comprising:
(a) obtaining, using the one or more processors, a plurality of sequence reads of one or more nucleic acids, wherein the one or more nucleic acids are derived from a sample obtained from an individual;
(b) analyzing, using the one or more processors, the plurality of sequence reads for the presence of a deletion of a methylthioadenosine phosphorylase (MTAP) gene, or of a portion thereof, and one or more mutations in a fibroblast growth factor receptor 3 (FGFR3) gene or in a phosphatase and tensin homolog (PTEN) gene; and
(c) detecting, using the one or more processors and based on the analyzing, a deletion of an MTAP gene, or of a portion thereof, and one or more mutations in an FGFR3 gene or in a PTEN gene, in the sample.

Exemplary Embodiment 130: The non-transitory computer readable storage medium of embodiment 129, wherein the sample is obtained from an individual having a urothelial bladder cancer (UCB).

Exemplary Embodiment 131: The non-transitory computer readable storage medium of embodiment 129 or embodiment 130, wherein the one or more mutations comprise one or more of an insertion, deletion or substitution of one or more nucleotides, a genomic rearrangement, an alteration in a promoter, a gene fusion, or a copy number alteration.

Exemplary Embodiment 132: The non-transitory computer readable storage medium of any one of embodiments 129-131, wherein the one or more mutations result in one or more of an insertion, deletion or substitution of one or more amino acid residues in a polypeptide encoded by the FGFR3 gene or the PTEN gene.

Exemplary Embodiment 133: The non-transitory computer readable storage medium of any one of embodiments 129-132, wherein the plurality of sequence reads is obtained by sequencing, whole exome sequencing, whole genome sequencing, gene-targeted sequencing, or next-generation sequencing.

Exemplary Embodiment 134: A system comprising:
a memory configured to store one or more program instructions; and
one or more processors configured to execute the one or more program instructions, wherein the one or more program instructions when executed by the one or more processors are configured to:
(a) obtain a plurality of sequence reads of one or more nucleic acids, wherein the one or more nucleic acids are derived from a sample obtained from an individual having a urothelial bladder cancer (UCB);
(b) analyze the plurality of sequence reads for the presence of a tumor mutational burden (TMB) of at least about 10 mut/Mb or at least about 20 mut/Mb, and/or one or more mutations in a retinoblastoma (RB1) gene; and
(c) detect, based on the analyzing, a TMB of at least about 10 mut/Mb or at least about 20 mut/Mb, and/or one or more mutations in an RB1 gene in the sample.

Exemplary Embodiment 135: A system comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acids, wherein the one or more nucleic acids are derived from a sample obtained from an individual;
(b) analyze the plurality of sequence reads for the presence of a tumor mutational burden (TMB) of at least about 10 mut/Mb or at least about 20 mut/Mb, and/or one or more mutations in a retinoblastoma (RB1) gene; and
(c) detect, based on the analyzing, a tumor mutational burden (TMB) of at least about 10 mut/Mb or at least about 20 mut/Mb, and/or one or more mutations in a retinoblastoma (RB1) gene.

Exemplary Embodiment 136: The system of embodiment 135, wherein the sample is obtained from an individual having a urothelial bladder cancer (UCB).

Exemplary Embodiment 137: The system of any one of embodiments 134-136, wherein the analyzing for the presence of a tumor mutational burden (TMB) is based on about 0.80 Mb of sequenced DNA.

Exemplary Embodiment 138: The system of any one of embodiments 134-136, wherein the analyzing for the presence of a tumor mutational burden (TMB) is based on between about 0.83 Mb and about 1.14 Mb of sequenced DNA.

Exemplary Embodiment 139: The system of any one of embodiments 134-136, wherein the analyzing for the presence of a tumor mutational burden (TMB) is based on up to about 1.1 Mb of sequenced DNA.

Exemplary Embodiment 140: The system of any one of embodiments 134-139, wherein the one or more mutations in the RB1 gene comprise one or more of an insertion, deletion or substitution of one or more nucleotides, a genomic rearrangement, an alteration in a promoter of the RB1 gene, a gene fusion, or a copy number alteration.

Exemplary Embodiment 141: The system of any one of embodiments 134-140, wherein the one or more mutations in the RB1 gene result in one or more of an insertion, deletion or substitution of one or more amino acid residues in a polypeptide encoded by the RB1 gene.

Exemplary Embodiment 142: The system of any one of embodiments 134-141, wherein the plurality of sequence reads is obtained by sequencing, whole exome sequencing, whole genome sequencing, gene-targeted sequencing, or next-generation sequencing.

Exemplary Embodiment 143: A non-transitory computer readable storage medium comprising one or more programs executable by one or more computer processors for performing a method, comprising:
(a) obtaining, using the one or more processors, a plurality of sequence reads of one or more nucleic acids, wherein the one or more nucleic acids are derived from a sample obtained from an individual;
(b) analyzing, using the one or more processors, the plurality of sequence reads for the presence of a tumor mutational burden (TMB) of at least about 10 mut/Mb or at least about 20 mut/Mb, and/or one or more mutations in a retinoblastoma (RB1) gene; and
(c) detecting, using the one or more processors and based on the analyzing, a tumor mutational burden (TMB) of at least about 10 mut/Mb or at least about 20 mut/Mb, and/or one or more mutations in a retinoblastoma (RB1) gene, in the sample.

Exemplary Embodiment 144: The non-transitory computer readable storage medium of embodiment 143, wherein the sample is obtained from an individual having a urothelial bladder cancer (UCB).

Exemplary Embodiment 145: The non-transitory computer readable storage medium of embodiment 143 or embodiment 144, wherein the analyzing for the presence of a tumor mutational burden (TMB) is based on about 0.80 Mb of sequenced DNA.

Exemplary Embodiment 146: The non-transitory computer readable storage medium of embodiment 143 or embodiment 144, wherein the analyzing for the presence of a tumor mutational burden (TMB) is based on between about 0.83 Mb and about 1.14 Mb of sequenced DNA.

Exemplary Embodiment 147: The non-transitory computer readable storage medium of embodiment 143 or embodiment 144, wherein the analyzing for the presence of a tumor mutational burden (TMB) is based on up to about 1.1 Mb of sequenced DNA.

Exemplary Embodiment 148: The non-transitory computer readable storage medium of any one of embodiments 143-147, wherein the one or more mutations in the RB1 gene comprise one or more of an insertion, deletion or substitution of one or more nucleotides, a genomic rearrangement, an alteration in a promoter of the RB1 gene, a gene fusion, or a copy number alteration.

Exemplary Embodiment 149: The non-transitory computer readable storage medium of any one of embodiments 143-148, wherein the one or more mutations in the RB1 gene result in one or more of an insertion, deletion or substitution of one or more amino acid residues in a polypeptide encoded by the RB1 gene.

Exemplary Embodiment 150: The non-transitory computer readable storage medium of any one of embodiments 143-149, wherein the plurality of sequence reads is obtained by sequencing, whole exome sequencing, whole genome sequencing, gene-targeted sequencing, or next-generation sequencing.

Exemplary Embodiment 151: An FGFR-targeted therapy for use in a method of treating or delaying progression of a UCB, wherein the method comprises administering an FGFR-targeted therapy to an individual having a UCB, wherein a deletion of an MTAP gene, or of a portion thereof, and one or more mutations in an FGFR3 gene are detected in a sample obtained from the individual.

Exemplary Embodiment 152: A PTEN-targeted therapy for use in a method of treating or delaying progression of a UCB, wherein the method comprises administering a PTEN-targeted therapy to an individual having a UCB, wherein a deletion of an MTAP gene, or of a portion thereof, and one or more mutations in a PTEN gene are detected in a sample obtained from the individual.

Exemplary Embodiment 153: An immunotherapy for use in a method of treating or delaying progression of a UCB, wherein the method comprises administering an immunotherapy to an individual having a UCB, wherein a TMB of at least about 10 mut/Mb or at least about 20 mut/Mb, and/or a PD-L1-positive UCB are detected in a sample obtained from the individual.

Exemplary Embodiment 154: The immunotherapy of embodiment 153, wherein the absence of a deletion of an MTAP gene, or of a portion thereof, is further detected in the sample.

Exemplary Embodiment 155: An RB1-targeted therapy for use in a method of treating or delaying progression of a UCB, wherein the method comprises administering an RB1-targeted therapy to an individual having a UCB, wherein one or more mutations in an RB1 gene are detected in a sample obtained from the individual.

Exemplary Embodiment 156: The RB1-targeted therapy of embodiment 155, wherein the absence of a deletion of an MTAP gene, or of a portion thereof, is further detected in the sample.

Exemplary Embodiment 157: An FGFR-targeted therapy for use in the manufacture of a medicament for treating or delaying progression of a UCB, wherein the medicament is to be administered to an individual having a UCB, wherein a deletion of an MTAP gene, or of a portion thereof, and one or more mutations in an FGFR3 gene have been detected in a sample obtained from the individual.

Exemplary Embodiment 158: A PTEN-targeted therapy for use in the manufacture of a medicament for treating or delaying progression of a UCB, wherein the medicament is to be administered to an individual having a UCB, wherein a deletion of an MTAP gene, or of a portion thereof, and one or more mutations in a PTEN gene have been detected in a sample obtained from the individual.

Exemplary Embodiment 159: An immunotherapy for use in the manufacture of a medicament for treating or delaying progression of a UCB, wherein the medicament is to be administered to an individual having a UCB, wherein a TMB of at least about 10 mut/Mb or at least about 20 mut/Mb, and/or a PD-L1-positive UCB have been detected in a sample obtained from the individual.

Exemplary Embodiment 160: The immunotherapy of embodiment 159, wherein the absence of a deletion of an MTAP gene, or of a portion thereof, is further detected in the sample.

Exemplary Embodiment 161: An RB1-targeted therapy for use in the manufacture of a medicament for treating or delaying progression of a UCB, wherein the medicament is to be administered to an individual having a UCB, wherein one or more mutations in an RB1 gene have been detected in a sample obtained from the individual.

Exemplary Embodiment 162: The RB1-targeted therapy of embodiment 161, wherein the absence of a deletion of an MTAP gene, or of a portion thereof, is further detected in the sample.

Exemplary Embodiment 163: A method of identifying an individual having a non-small cell lung cancer (NSCLC) who may benefit from a treatment comprising an epidermal growth factor receptor (EGFR)-targeted therapy or a SMARCA4-targeted therapy, the method comprising detecting in a sample from the individual a deletion of a methylthioadenosine phosphorylase (MTAP) gene, or
of a portion thereof, and one or more mutations in an EGFR gene or in a SMARCA4 gene, wherein:
(a) the presence of the deletion of the MTAP gene, or of the portion thereof, and one or more mutations in the EGFR gene in the sample identifies the individual as one who may benefit from a treatment comprising an EGFR-targeted therapy; or
(b) the presence of the deletion of the MTAP gene, or of the portion thereof, and one or more mutations in the SMARCA4 gene in the sample identifies the individual as one who may benefit from a treatment comprising a SMARCA4-targeted therapy.

Exemplary Embodiment 164: A method of selecting a treatment for an individual having a NSCLC, the method comprising detecting a deletion of an MTAP gene, or of a portion thereof, and one or more mutations in an EGFR gene or in a SMARCA4 gene in a sample from the individual, wherein:
(a) the presence of the deletion of the MTAP gene, or of the portion thereof, and one or more mutations in the EGFR gene in the sample identifies the individual as one who may benefit from a treatment comprising an EGFR-targeted therapy; or
(b) the presence of the deletion of the MTAP gene, or of the portion thereof, and one or more mutations in the SMARCA4 gene in the sample identifies the individual as one who may benefit from a treatment comprising a SMARCA4-targeted therapy.

Exemplary Embodiment 165: The method of embodiment 163 or embodiment 164, wherein the presence in the sample of the deletion of the MTAP gene, or of the portion thereof, and the one or more mutations in the EGFR gene or in the SMARCA4 gene identifies the individual as one who may benefit from an additional treatment comprising a protein arginine methyltransferase 5 (PRMT5)-targeted therapy.

Exemplary Embodiment 166: A method of identifying one or more treatment options for an individual having a NSCLC, the method comprising:
(a) detecting a deletion of an MTAP gene, or of a portion thereof, and one or more mutations in an EGFR gene or in a SMARCA4 gene in a sample from the individual; and
(b) generating a report, wherein the report comprises:
(i) one or more treatment options identified for the individual based at least in part on the presence of the deletion of the MTAP gene, or of the portion thereof, and one or more mutations in the EGFR gene in the sample, wherein the one or more treatment options comprise an EGFR-targeted therapy; or
(ii) one or more treatment options identified for the individual based at least in part on the presence of the deletion of the MTAP gene, or of the portion thereof, and one or more mutations in the SMARCA4 gene in the sample, wherein the one or more treatment options comprise a SMARCA4-targeted therapy.

Exemplary Embodiment 167: A method of identifying one or more treatment options for an individual having a NSCLC, the method comprising acquiring knowledge of a deletion of an MTAP gene, or of a portion thereof, and one or more mutations in an EGFR gene or in a SMARCA4 gene in a sample from the individual; and generating a report comprising:
(a) one or more treatment options identified for the individual based at least in part on the knowledge of a deletion of the MTAP gene, or of a portion thereof, and one or more mutations in the EGFR gene in the sample, wherein the one or more treatment options comprise an EGFR-targeted therapy; or
(b) one or more treatment options identified for the individual based at least in part on the knowledge of a deletion of the MTAP gene, or of a portion thereof, and one or more mutations in the SMARCA4 gene in the sample, wherein the one or more treatment options comprise a SMARCA4-targeted therapy.

Exemplary Embodiment 168: The method of embodiment 166 or embodiment 167, wherein the one or more treatment options identified for the individual further comprise a PRMT5-targeted therapy.

Exemplary Embodiment 169: A method of selecting a treatment for an individual having a NSCLC, comprising acquiring knowledge of a deletion of an MTAP gene, or of a portion thereof, and one or more mutations in an EGFR gene or in a SMARCA4 gene in a sample from the individual, wherein:
(a) responsive to the acquisition of knowledge of a deletion of an MTAP gene, or of a portion thereof, and one or more mutations in an EGFR gene in the sample: (i) the individual is classified as a candidate to receive a treatment comprising an EGFR-targeted therapy; and/or (ii) the individual is identified as likely to respond to a treatment that comprises an EGFR-targeted therapy; or
(b) responsive to the acquisition of knowledge of a deletion of an MTAP gene, or of a portion thereof, and one or more mutations in a SMARCA4 gene in the sample: (i) the individual is classified as a candidate to receive a treatment comprising a SMARCA4-targeted therapy; and/or (ii) the individual is identified as likely to respond to a treatment that comprises a SMARCA4-targeted therapy.

Exemplary Embodiment 170: The method of embodiment 169, wherein the treatment further comprises a PRMT5-targeted therapy.

Exemplary Embodiment 171: A method of predicting survival of an individual having a NSCLC treated with a treatment comprising an EGFR-targeted therapy or a SMARCA4-targeted therapy, the method comprising acquiring knowledge of a deletion of an MTAP gene, or of a portion thereof, and one or more mutations in an EGFR gene or in a SMARCA4 gene in a sample from the individual, wherein responsive to the acquisition of said knowledge, the individual is predicted to have longer survival after treatment with the EGFR-targeted therapy or the SMARCA4-targeted therapy, as compared to an individual whose NSCLC does not exhibit a deletion of an MTAP gene, or of a portion thereof, and one or more mutations in an EGFR gene or in a SMARCA4 gene.

Exemplary Embodiment 172: A method of treating or delaying progression of a NSCLC, comprising:
(a) acquiring knowledge of a deletion of an MTAP gene, or of a portion thereof, and one or more mutations in an EGFR gene in a sample from an individual, and responsive to said knowledge, administering to the individual an effective amount of a treatment comprising an EGFR-targeted therapy; or
(b) acquiring knowledge of a deletion of an MTAP gene, or of a portion thereof, and one or more mutations in a SMARCA4 gene in a sample from an individual, and responsive to said knowledge, administering to the individual an effective amount of a treatment comprising a SMARCA4-targeted therapy.

Exemplary Embodiment 173: A method of treating or delaying progression of a NSCLC, comprising:
(a) responsive to acquiring knowledge of a deletion of an MTAP gene, or of a portion thereof, and one or more mutations in an EGFR gene in a sample from an individual, administering to the individual an effective amount of a treatment comprising an EGFR-targeted therapy; or
(b) responsive to acquiring knowledge of a deletion of an MTAP gene, or of a portion thereof, and one or more mutations in a SMARCA4 gene in a sample from an individual, administering to the individual an effective amount of a treatment comprising a SMARCA4-targeted therapy.

Exemplary Embodiment 174: The method of any one of embodiments 167-173, wherein the acquiring knowledge of the deletion of the MTAP gene, or of the portion thereof, and one or more mutations in the EGFR gene or in the SMARCA4 gene comprises detecting the deletion of the MTAP gene, or of the portion thereof, and the one or more mutations in the EGFR gene or in the SMARCA4 gene in the sample.

Exemplary Embodiment 175: A method of treating or delaying progression of a NSCLC, comprising:
(a) detecting a deletion of an MTAP gene, or of a portion thereof, and one or more mutations in an EGFR gene in a sample from an individual, and administering to the individual an effective amount of a treatment comprising an EGFR-targeted therapy; or
(b) detecting a deletion of an MTAP gene, or of a portion thereof, and one or more mutations in a SMARCA4 gene in a sample from an individual, and administering to the individual an effective amount of a treatment comprising a SMARCA4-targeted therapy.

Exemplary Embodiment 176: A method of detecting the presence or absence of a NSCLC in an individual, comprising:
(a) detecting the presence or absence of a NSCLC in a sample from an individual; and
(i) detecting the presence or absence of a deletion of an MTAP gene, or of a portion thereof, and one or more mutations in an EGFR gene in the sample, or
(ii) detecting the presence or absence of a deletion of an MTAP gene, or of a portion thereof, and one or more mutations in a SMARCA4 gene in the sample.

Exemplary Embodiment 177: A method of assessing a NSCLC in an individual, the method comprising:
(a) detecting a deletion of an MTAP gene, or of a portion thereof, and one or more mutations in an EGFR gene or in a SMARCA4 gene in a sample from the individual; and
(b) providing an assessment of the deletion of the MTAP gene, or of the portion thereof, and the one or more mutations in the EGFR gene or in the SMARCA4 gene.

Exemplary Embodiment 178: A method of detecting a deletion of an MTAP gene, or of a portion thereof, and one or more mutations in an EGFR gene or in a SMARCA4 gene, the method comprising detecting the deletion of the MTAP gene, or of the portion thereof, and the one or more mutations in the EGFR gene or in the SMARCA4 gene in a sample from an individual having a NSCLC.

Exemplary Embodiment 179: A method of detecting a deletion of an MTAP gene, or of a portion thereof, and one or more mutations in an EGFR gene or in a SMARCA4 gene, the method comprising:
(a) providing a sample from an individual having a NSCLC, wherein the sample comprises one or more nucleic acids;
(b) preparing a nucleic acid sequencing library from the one or more nucleic acids in the sample;
(c) amplifying said library using a polymerase chain reaction (PCR);
(d) selectively enriching for one or more nucleic acids comprising MTAP, EGFR, or SMARCA4 gene nucleotide sequences in said library to produce an enriched sample;
(e) sequencing the enriched sample, thereby producing a plurality of sequencing reads;
(f) analyzing the plurality of sequencing reads for the presence of a deletion of an MTAP gene, or of a portion thereof, and one or more mutations in an EGFR gene or in a SMARCA4 gene;
(g) detecting, based on the analyzing step, a deletion of an MTAP gene, or of a portion thereof, and one or more mutations in an EGFR gene or in a SMARCA4 gene in the sample from the individual.

Exemplary Embodiment 180: The method of any one of embodiments 163-166 and 174-179, further comprising selectively enriching for one or more of: (a) one or more nucleic acids comprising nucleotide sequences that comprise the deletion of the MTAP gene, or of the portion thereof, (b) one or more nucleic acids comprising nucleotide sequences that comprise the one or more mutations in the EGFR gene, or (c) one or more nucleic acids comprising nucleotide sequences that comprise the one or more mutations in the SMARCA4 gene; wherein the selectively enriching produces an enriched sample.

Exemplary Embodiment 181: A method of treating or delaying progression of a NSCLC, comprising:
(a) administering to an individual having a NSCLC an effective amount of a treatment comprising an EGFR-targeted therapy, wherein the NSCLC comprises a deletion of an MTAP gene, or of a portion thereof, and one or more mutations in an EGFR gene; or
(b) administering to an individual having a NSCLC an effective amount of a treatment comprising a SMARCA4-targeted therapy, wherein the NSCLC comprises a deletion of an MTAP gene, or of a portion thereof, and one or more mutations in a SMARCA4 gene.

Exemplary Embodiment 182: The method of any one of embodiments 163-180, further comprising acquiring knowledge of or detecting in the sample a deletion of a CDKN2A gene, or of a portion thereof; and/or a deletion of a CDKN2B gene, or of a portion thereof.

Exemplary Embodiment 183: The method of any one of embodiments 163-180 and 182, comprising acquiring knowledge of or detecting in the sample one or more mutations in one or more genes, wherein the one or more genes comprise TP53, KRAS, ALK, ROS1, NTRK1, STK11, KEAP1, PIK3CA, PTEN, MET, ERBB2, BRAF, or RB1.

Exemplary Embodiment 184: The method of any one of embodiments 163-183, wherein the individual has one or more mutations in one or more genes, wherein the one or more genes comprise TP53, KRAS, ALK, ROS1, NTRK1, STK11, KEAP1, PIK3CA, PTEN, MET, ERBB2, BRAF, or RB1.

Exemplary Embodiment 185: The method of embodiment 183 or embodiment 184, further comprising administering to the individual an effective amount of a treatment comprising an anti-cancer therapy.

Exemplary Embodiment 186: The method of any one of embodiments 183-185, further comprising selectively enriching for one or more nucleic acids comprising nucleotide sequences that comprise one or more mutations in one or more genes, wherein the one or more genes comprise TP53, KRAS, ALK, ROS1, NTRK1, STK11, KEAP1, PIK3CA, PTEN, MET, ERBB2, BRAF, or RB1, to produce an enriched sample.

Exemplary Embodiment 187: The method of any one of embodiments 183-186, wherein the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 188: The method of any one of embodiments 163-187, wherein the individual has a deletion of a CDKN2A gene, or of a portion thereof; and/or a deletion of a CDKN2B gene, or of a portion thereof.

Exemplary Embodiment 189: The method of embodiment 188, wherein the individual has a heterozygous or a homozygous deletion of a CDKN2A gene, or of a portion thereof; and/or a heterozygous or a homozygous deletion of a CDKN2B gene, or of a portion thereof.

Exemplary Embodiment 190: The method of any one of embodiments 163-189, wherein the individual has a heterozygous or a homozygous deletion of the MTAP gene, or of a portion thereof.

Exemplary Embodiment 191: The method of any one of embodiments 172-175 and 180-190, further comprising administering to the individual a PRMT5-targeted therapy.

Exemplary Embodiment 192: The method of any one of embodiments 163-191, wherein the individual is being treated with a PRMT5-targeted therapy.

Exemplary Embodiment 193: The method of any one of embodiments 165, 168, 170, and 191-192, wherein the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

Exemplary Embodiment 194: The method of any one of embodiments 163-193, wherein the one or more mutations in the EGFR gene or the SMARCA4 gene comprise one or more of an insertion, deletion or substitution of one or more nucleotides, a genomic rearrangement, an alteration in a promoter, a gene fusion, or a copy number alteration.

Exemplary Embodiment 195: The method of any one of embodiments 163-194, wherein the one or more mutations in the EGFR gene or the SMARCA4 gene result in one or more of an insertion, deletion or substitution of one or more amino acid residues in a polypeptide encoded by the EGFR gene or the SMARCA4 gene.

Exemplary Embodiment 196: The method of any one of embodiments 163-175 and 180-195, wherein the EGFR-targeted therapy or the SMARCA4-targeted therapy is a small molecule inhibitor, an antibody, a cellular therapy, or a nucleic acid.

Exemplary Embodiment 197: The method of embodiment 196, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 198: The method of embodiment 196, wherein the nucleic acid comprises a double-stranded RNA (dsRNA), a small interfering RNA (siRNA), or a small hairpin RNA (shRNA).

Exemplary Embodiment 199: The method of any one of embodiments 163-175 and 180-196, wherein the EGFR-targeted therapy comprises one or more of an EGFR inhibitor, an HSP90 inhibitor, a VEGFR/EGFR dual inhibitor, a MEK inhibitor, or a Raf inhibitor.

Exemplary Embodiment 200: The method of any one of embodiments 163-175 and 180-196, wherein the SMARCA4-targeted therapy comprises one or more of a CDK4/6 inhibitor, an Aurora kinase (AURK) inhibitor, an ATR inhibitor, an EZH2 inhibitor, a KDM6 inhibitor, a kinase inhibitor, a cisplatin-based chemotherapy, or an immune checkpoint inhibitor.

Exemplary Embodiment 201: The method of any one of embodiments 163-166, 174-180, and 182-200, wherein the deletion of the MTAP gene or of the portion thereof, or the one or more mutations in the EGFR gene or the SMARCA4 gene are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 202: The method of any one of embodiments 163-166, 174-180, and 182-200, wherein the deletion of the MTAP gene or of the portion thereof, or the one or more mutations in the EGFR gene or the SMARCA4 gene are detected in a polypeptide encoded by the MTAP gene, the EGFR gene, or the SMARCA4 gene.

Exemplary Embodiment 203: The method of embodiment 202, wherein the deletion of the MTAP gene or of the portion thereof, or the one or more mutations in the EGFR gene or the SMARCA4 gene are detected in the sample by one or more of: immunoblotting, enzyme linked immunosorbent assay (ELISA), immunohistochemistry, or mass spectrometry.

Exemplary Embodiment 204: The method of any one of embodiments 182-203, wherein the deletion of the CDKN2A gene or of the portion thereof, and/or the deletion of the CDKN2B gene or of the portion thereof, are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 205: The method of any one of embodiments 182-203, wherein the deletion of the CDKN2A gene or of the portion thereof, and/or the deletion of the CDKN2B gene or of the portion thereof, are detected in a polypeptide encoded by the CDKN2A gene or the CDKN2B gene.

Exemplary Embodiment 206: The method of embodiment 205, wherein the deletion of the CDKN2A gene or of the portion thereof, and/or the deletion of the CDKN2B gene or of the portion thereof, are detected in the sample by one or more of: immunoblotting, enzyme linked immunosorbent assay (ELISA), immunohistochemistry, or mass spectrometry.

Exemplary Embodiment 207: A method of identifying an individual having a non-small cell lung cancer (NSCLC) who may benefit from a treatment comprising an immunotherapy, a retinoblastoma (RB1)-targeted therapy, a KRAS-targeted therapy, or a TP53-targeted therapy, the method comprising detecting in a sample from the individual one or more of: (a) a tumor mutational burden (TMB) of at least about 10 mut/Mb or at least about 20 mut/Mb, (b) a PD-L1-positive NSCLC, or (c) one or more mutations in an RB1 gene, a KRAS gene, or a TP53 gene; wherein:
(i) the presence in the sample of a TMB of at least about 10 mut/Mb or at least about 20 mut/Mb, or of a PD-L1-positive NSCLC identifies the individual as one who may benefit from a treatment comprising an immunotherapy;
(ii) the presence in the sample of one or more mutations in the RB1 gene identifies the individual as one who may benefit from a treatment comprising an RB1-targeted therapy;
(iii) the presence in the sample of one or more mutations in the KRAS gene identifies the individual as one who may benefit from a treatment comprising a KRAS-targeted therapy; or
(iv) the presence in the sample of one or more mutations in the TP53 gene identifies the individual as one who may benefit from a treatment comprising a TP53-targeted therapy.

Exemplary Embodiment 208: A method of selecting a treatment for an individual having a NSCLC, the method comprising detecting one or more of: (a) a TMB of at least about 10 mut/Mb or at least about 20 mut/Mb, (b) a PD-L1-positive NSCLC, or (c) one or more mutations in an RB1 gene, a KRAS gene, or a TP53 gene, in a sample from the individual; wherein:
(i) the presence in the sample of a TMB of at least about 10 mut/Mb or at least about 20 mut/Mb, or of a PD-L1-positive NSCLC identifies the individual as one who may benefit from a treatment comprising an immunotherapy;
(ii) the presence in the sample of one or more mutations in the RB1 gene identifies the individual as one who may benefit from a treatment comprising an RB1-targeted therapy;
(iii) the presence in the sample of one or more mutations in the KRAS gene identifies the individual as one who may benefit from a treatment comprising a KRAS-targeted therapy; or
(iv) the presence in the sample of one or more mutations in the TP53 gene identifies the individual as one who may benefit from a treatment comprising a TP53-targeted therapy.

Exemplary Embodiment 209: A method of identifying one or more treatment options for an individual having a NSCLC, the method comprising:
(a) detecting in a sample from the individual one or more of: (i) a TMB of at least about 10 mut/Mb or at least about 20 mut/Mb, (ii) a PD-L1-positive NSCLC, or (iii) one or more mutations in an RB1 gene, a KRAS gene, or a TP53 gene; and
(b) generating a report, wherein the report comprises:
(i) one or more treatment options identified for the individual based at least in part on the presence of a TMB of at least about 10 mut/Mb or at least about 20 mut/Mb, or of a PD-L1-positive NSCLC in the sample, wherein the one or more treatment options comprise an immunotherapy;
(ii) one or more treatment options identified for the individual based at least in part on the presence of one or more mutations in the RB1 gene in the sample, wherein the one or more treatment options comprise an RB1-targeted therapy;
(iii) one or more treatment options identified for the individual based at least in part on the presence of one or more mutations in the KRAS gene in the sample, wherein the one or more treatment options comprise a KRAS-targeted therapy; or
(iv) one or more treatment options identified for the individual based at least in part on the presence of one or more mutations in the TP53 gene in the sample, wherein the one or more treatment options comprise a TP53-targeted therapy.

Exemplary Embodiment 210: A method of identifying one or more treatment options for an individual having a NSCLC, the method comprising acquiring knowledge of one or more of: (a) a TMB of at least about 10 mut/Mb or at least about 20 mut/Mb, (b) a PD-L1-positive NSCLC, or (c) one or more mutations in an RB1 gene, a KRAS gene, or a TP53 gene, in a sample from the individual; and generating a report comprising:
(i) one or more treatment options identified for the individual based at least in part on the knowledge of a TMB of at least about 10 mut/Mb or at least about 20 mut/Mb, or of a PD-L1-positive NSCLC in the sample, wherein the one or more treatment options comprise an immunotherapy;
(ii) one or more treatment options identified for the individual based at least in part on the knowledge of one or more mutations in the RB1 gene in the sample, wherein the one or more treatment options comprise an RB1-targeted therapy;
(iii) one or more treatment options identified for the individual based at least in part on the knowledge of one or more mutations in the KRAS gene in the sample, wherein the one or more treatment options comprise a KRAS-targeted therapy; or
(iv) one or more treatment options identified for the individual based at least in part on the knowledge of one or more mutations in the TP53 gene in the sample, wherein the one or more treatment options comprise a TP53-targeted therapy.

Exemplary Embodiment 211: A method of selecting a treatment for an individual having a NSCLC, comprising acquiring knowledge of one or more of: (a) a TMB of at least about 10 mut/Mb or at least about 20 mut/Mb, (b) a PD-L1-positive NSCLC, or (c) one or more mutations in an RB1 gene, a KRAS gene, or a TP53 gene, in a sample from the individual, wherein:
(a) responsive to acquisition of knowledge of a TMB of at least about 10 mut/Mb or at least about 20 mut/Mb, or of a PD-L1-positive NSCLC in the sample: (i) the individual is classified as a candidate to receive a treatment comprising an immunotherapy; and/or (ii) the individual is identified as likely to respond to a treatment that comprises an immunotherapy;
(b) responsive to acquisition of knowledge of one or more mutations in the RB1 gene in the sample: (i) the individual is classified as a candidate to receive a treatment comprising an RB1-targeted therapy; and/or (ii) the individual is identified as likely to respond to a treatment that comprises an RB1-targeted therapy;
(c) responsive to acquisition of knowledge of one or more mutations in the KRAS gene in the sample:
(i) the individual is classified as a candidate to receive a treatment comprising a KRAS-targeted therapy; and/or (ii) the individual is identified as likely to respond to a treatment that comprises a KRAS-targeted therapy; or
(d) responsive to acquisition of knowledge of one or more mutations in the TP53 gene in the sample:
(i) the individual is classified as a candidate to receive a treatment comprising a TP53-targeted therapy; and/or (ii) the individual is identified as likely to respond to a treatment that comprises a TP53-targeted therapy.

Exemplary Embodiment 212: A method of predicting survival of an individual having NSCLC treated with a treatment comprising an immunotherapy, an RB1-targeted therapy, a KRAS-targeted therapy, or a TP53-targeted therapy, the method comprising acquiring knowledge of one or more of: (a) a TMB of at least about 10 mut/Mb or at least about 20 mut/Mb, (b) a PD-L1-positive NSCLC, or (c) one or more mutations in an RB1 gene, a KRAS gene, or a TP53 gene, in a sample from the individual; wherein responsive to acquisition of said knowledge, the individual is predicted to have longer survival after treatment with the immunotherapy, RB1-targeted therapy, KRAS-targeted therapy, or TP53-targeted therapy, as compared to an individual whose NSCLC does not exhibit a TMB of at least about 10 mut/Mb or at least about 20 mut/Mb, a PD-L1-positive NSCLC, or one or more mutations in the RB1 gene, KRAS gene, or TP53 gene.

Exemplary Embodiment 213: A method of treating or delaying progression of a NSCLC, comprising:
(a) acquiring knowledge of a TMB of at least about 10 mut/Mb or at least about 20 mut/Mb, or a PD-L1-positive NSCLC in a sample from an individual, and responsive to said knowledge, administering to the individual an effective amount of a treatment comprising an immunotherapy;
(b) acquiring knowledge of one or more mutations in an RB1 gene in a sample from an individual, and responsive to said knowledge, administering to the individual an effective amount of a treatment comprising an RB1-targeted therapy;
(c) acquiring knowledge of one or more mutations in a KRAS gene in a sample from an individual, and responsive to said knowledge, administering to the individual an effective amount of a treatment comprising a KRAS-targeted therapy; or
(d) acquiring knowledge of one or more mutations in a TP53 gene in a sample from an individual, and responsive to said knowledge, administering to the individual an effective amount of a treatment comprising a TP53-targeted therapy.

Exemplary Embodiment 214: A method of treating or delaying progression of a NSCLC, comprising:
(a) responsive to acquiring knowledge of a TMB of at least about 10 mut/Mb or at least about 20 mut/Mb, or a PD-L1-positive NSCLC in a sample from an individual, administering to the individual an effective amount of a treatment comprising an immunotherapy;
(b) responsive to acquiring knowledge of one or more mutations in an RB1 gene in a sample from an individual, administering to the individual an effective amount of a treatment comprising an RB1-targeted therapy;
(c) responsive to acquiring knowledge of one or more mutations in a KRAS gene in a sample from an individual, administering to the individual an effective amount of a treatment comprising a KRAS-targeted therapy; or
(d) responsive to acquiring knowledge of one or more mutations in a TP53 gene in a sample from an individual, administering to the individual an effective amount of a treatment comprising a TP53-targeted therapy.

Exemplary Embodiment 215: The method of any one of embodiments 210-214, wherein acquiring knowledge of a TMB of at least about 10 mut/Mb or at least about 20 mut/Mb, a PD-L1-positive NSCLC, or one or more mutations in an RB1 gene, a KRAS gene, or a TP53 gene comprises detecting the TMB of at least about 10 mut/Mb or at least about 20 mut/Mb, the PD-L1-positive NSCLC, or the one or more mutations in the RB1 gene, KRAS gene, or TP53 gene in the sample.

Exemplary Embodiment 216: A method of treating or delaying progression of a NSCLC, comprising:
(a) detecting a TMB of at least about 10 mut/Mb or at least about 20 mut/Mb, or a PD-L1-positive NSCLC in a sample from an individual, and administering to the individual an effective amount of a treatment comprising an immunotherapy;
(b) detecting one or more mutations in an RB1 gene in a sample from an individual, and administering to the individual an effective amount of a treatment comprising an RB1-targeted therapy; (c) detecting one or more mutations in a KRAS gene in a sample from an individual, and administering to the individual an effective amount of a treatment comprising a KRAS-targeted therapy; or
(d) detecting one or more mutations in a TP53 gene in a sample from an individual, and administering to the individual an effective amount of a treatment comprising a TP53-targeted therapy.

Exemplary Embodiment 217: A method of detecting the presence or absence of a NSCLC in an individual, comprising:
(a) detecting the presence or absence of a NSCLC in a sample from an individual; and
(i) detecting the presence or absence of a TMB of at least about 10 mut/Mb or at least about 20 mut/Mb, or a PD-L1-positive NSCLC in the sample,
(ii) detecting the presence or absence of one or more mutations in an RB1 gene in the sample,
(iii) detecting the presence or absence of one or more mutations in a KRAS gene in the sample, or
(iv) detecting the presence or absence of one or more mutations in a TP53 gene in the sample.

Exemplary Embodiment 218: A method of assessing a NSCLC in an individual, the method comprising:
(a) detecting one or more of: (i) a TMB of at least about 10 mut/Mb or at least about 20 mut/Mb, (ii) a PD-L1-positive NSCLC, or (iii) one or more mutations in an RB1 gene, a KRAS gene, or a TP53 gene, in a sample from the individual; and
(b) providing an assessment of the TMB of at least about 10 mut/Mb or at least about 20 mut/Mb, the PD-L1-positive NSCLC, or the one or more mutations in the RB1 gene, KRAS gene, or TP53 gene in the sample.

Exemplary Embodiment 219: A method of detecting a TMB of at least about 10 mut/Mb or at least about 20 mut/Mb, a PD-L1-positive NSCLC, or one or more mutations in an RB1 gene, a KRAS gene, or a TP53 gene, the method comprising detecting the TMB of at least about 10 mut/Mb or at least about 20 mut/Mb, the PD-L1-positive NSCLC, or the one or more mutations in the RB1 gene, KRAS gene, or TP53 gene in a sample from an individual having a NSCLC.

Exemplary Embodiment 220: A method of detecting one or more mutations in an RB1 gene, a KRAS gene, or a TP53 gene, the method comprising:
(a) providing a sample from an individual having a NSCLC, wherein the sample comprises one or more nucleic acids;
(b) preparing a nucleic acid sequencing library from the one or more nucleic acids in the sample;
(c) amplifying said library using a polymerase chain reaction (PCR);
(d) selectively enriching for one or more nucleic acids comprising RB1 gene, KRAS gene, or TP53 gene nucleotide sequences in said library to produce an enriched sample;
(e) sequencing the enriched sample, thereby producing a plurality of sequencing reads;
(f) analyzing the plurality of sequencing reads for the presence of one or more mutations in an RB1 gene, a KRAS gene, or a TP53 gene;
(g) detecting, based on the analyzing step, one or more mutations in an RB1 gene, a KRAS gene, or a TP53 gene in the sample from the individual.

Exemplary Embodiment 221: A method of detecting a TMB of at least about 10 mut/Mb or at least about 20 mut/Mb, the method comprising:
(a) providing a sample from an individual having a NSCLC, wherein the sample comprises one or more nucleic acids;
(b) preparing a nucleic acid sequencing library from the one or more nucleic acids in the sample;
(c) amplifying said library using a polymerase chain reaction (PCR);
(d) sequencing said library, thereby producing a plurality of sequencing reads;
(e) analyzing the plurality of sequencing reads for the presence of a TMB of at least about 10 mut/Mb or at least about 20 mut/Mb;
(f) detecting, based on the analyzing step, a TMB of at least about 10 mut/Mb or at least about 20 mut/Mb in the sample from the individual.

Exemplary Embodiment 222: A method of treating or delaying progression of a NSCLC, comprising:
(a) administering to an individual having a NSCLC an effective amount of a treatment comprising an immunotherapy, wherein the NSCLC comprises a TMB of at least about 10 mut/Mb or at least about 20 mut/Mb, or wherein the NSCLC is PD-L1-positive;
(b) administering to an individual having a NSCLC an effective amount of a treatment comprising an RB1-targeted therapy, wherein the NSCLC comprises one or more mutations in an RB1 gene;
(c) administering to an individual having a NSCLC an effective amount of a treatment comprising a KRAS-targeted therapy, wherein the NSCLC comprises one or more mutations in a KRAS gene; or
(d) administering to an individual having a NSCLC an effective amount of a treatment comprising a TP53-targeted therapy, wherein the NSCLC comprises one or more mutations in a TP53 gene.

Exemplary Embodiment 223: The method of any one of embodiments 207-221, further comprising acquiring knowledge of the absence of a deletion of an MTAP gene, or of a portion thereof, in the sample.

Exemplary Embodiment 224: The method of embodiment 223, wherein acquiring knowledge of the absence of a deletion of an MTAP gene, or of a portion thereof, comprises detecting the absence of a deletion of an MTAP gene, or of a portion thereof, in the sample.

Exemplary Embodiment 225: The method of any one of embodiments 207-221 and 223-224, further comprising detecting the absence of a deletion of an MTAP gene, or of a portion thereof, in the sample.

Exemplary Embodiment 226: The method of any one of embodiments 207-221 and 223-225, comprising acquiring knowledge of or detecting in the sample one or more mutations in one or more genes, wherein the one or more genes comprise EGFR, ALK, ROS1, NTRK1, STK11, KEAP1, PIK3CA, SMARCA4, PTEN, MET, ERBB2, or BRAF.

Exemplary Embodiment 227: The method of any one of embodiments 207-226, wherein the individual has one or more mutations in one or more genes, wherein the one or more genes comprise EGFR, ALK, ROS1, NTRK1, STK11, KEAP1, PIK3CA, SMARCA4, PTEN, MET, ERBB2, or BRAF.

Exemplary Embodiment 228: The method of embodiment 226 or embodiment 227, further comprising administering to the individual an effective amount of a treatment comprising anti-cancer therapy.

Exemplary Embodiment 229: The method of embodiment 226 or embodiment 227, comprising selectively enriching for one or more nucleic acids comprising nucleotide sequences that comprise one or more mutations in one or more genes to produce an enriched sample, wherein the one or more genes comprise EGFR, ALK, ROS1, NTRK1, STK11, KEAP1, PIK3CA, SMARCA4, PTEN, MET, ERBB2, or BRAF.

Exemplary Embodiment 230: The method of any one of embodiments 226-229, wherein the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 231: The method of embodiment 226 or embodiment 227, wherein the one or more mutations in the one or more genes are detected in a polypeptide encoded by the one or more genes.

Exemplary Embodiment 232: The method of embodiment 231, wherein the one or more mutations in the one or more genes are detected in the sample by one or more of immunoblotting, enzyme linked immunosorbent assay (ELISA), immunohistochemistry, or mass spectrometry.

Exemplary Embodiment 233: The method of embodiment 224 or embodiment 225, further comprising selectively enriching for one or more nucleic acids comprising MTAP nucleotide sequences to produce an enriched sample.

Exemplary Embodiment 234: The method of any one of embodiments 224-233, wherein the absence of a deletion of an MTAP gene, or of a portion thereof, is detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 235: The method of any one of embodiments 207-234, wherein the individual does not have a deletion of an MTAP gene, or of a portion thereof.

Exemplary Embodiment 236: The method of any one of embodiments 207-209, 215-220, and 224-235, further comprising selectively enriching for one or more nucleic acids comprising nucleotide sequences that comprise the one or more mutations in the RB1 gene, KRAS gene, or TP53 gene to produce an enriched sample.

Exemplary Embodiment 237: The method of any one of embodiments 207-220 and 222-236, wherein the one or more mutations in the RB1 gene, KRAS gene, or TP53 gene comprise one or more of an insertion, deletion or substitution of one or more nucleotides, a genomic rearrangement, an alteration in a promoter of the RB1 gene, KRAS gene, or TP53 gene, a gene fusion, or a copy number alteration.

Exemplary Embodiment 238: The method of any one of embodiments 207-220 and 222-237, wherein the one or more mutations in the RB1 gene, KRAS gene, or TP53 gene result in one or more of an insertion, deletion or substitution of one or more amino acid residues in a polypeptide encoded by the RB1 gene, KRAS gene, or TP53 gene.

Exemplary Embodiment 239: The method of any one of embodiments 207-220 and 222-238, wherein the one or more mutations in the KRAS gene result in a G12C amino acid substitution in a KRAS polypeptide encoded by the KRAS gene.

Exemplary Embodiment 240: The method of any one of embodiments 207-209, 215-220, and 224-239, wherein the one or more mutations in the RB1 gene, KRAS gene, or TP53 gene are detected in the sample by one or more of: an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 241: The method of any one of embodiments 207-209, 215-220, and 224-239, wherein the one or more mutations in the RB1 gene, KRAS gene, or TP53 gene are detected in a polypeptide encoded by the RB1 gene, KRAS gene, or TP53 gene.

Exemplary Embodiment 242: The method of embodiment 241, wherein the one or more mutations in the RB1 gene, KRAS gene, or TP53 gene are detected in the sample by one or more of immunoblotting, enzyme linked immunosorbent assay (ELISA), immunohistochemistry, or mass spectrometry.

Exemplary Embodiment 243: The method of any one of embodiments 207-209, 215-219, 221, and 224-242, wherein detecting a TMB of at least about 10 mut/Mb or at least about 20 mut/Mb comprises measuring the level of TMB in the sample.

Exemplary Embodiment 244: The method of embodiment 243, wherein TMB is measured in the sample by whole exome sequencing, whole genome sequencing, or gene-targeted sequencing.

Exemplary Embodiment 245: The method of embodiment 243 or embodiment 244, wherein TMB is measured on about 0.80 Mb of sequenced DNA.

Exemplary Embodiment 246: The method of embodiment 243 or embodiment 244, wherein TMB is measured on between about 0.83 Mb and about 1.14 Mb of sequenced DNA.

Exemplary Embodiment 247: The method of embodiment 243 or embodiment 244, wherein TMB is measured on about 1.1 Mb of sequenced DNA.

Exemplary Embodiment 248: The method of embodiment 243 or embodiment 244, wherein TMB is measured on up to about 1.1 Mb of sequenced DNA.

Exemplary Embodiment 249: The method of any one of embodiments 207-209, 215-219, and 224-248, wherein detecting a PD-L1-positive NSCLC comprises measuring the level of PD-L1 expression in the sample.

Exemplary Embodiment 250: The method of embodiment 249, wherein the level of PD-L1 expression is measured using an immunohistochemistry assay.

Exemplary Embodiment 251: The method of embodiment 249 or embodiment 250, wherein the level of PD-L1 expression is determined based on PD-L1 expression on tumor cells.

Exemplary Embodiment 252: The method of embodiment 251, wherein the PD-L1-positive NSCLC comprises a tumor proportion score (TPS) of between about 1% and about 49%.

Exemplary Embodiment 253: The method of embodiment 251, wherein the PD-L1-positive NSCLC comprises a tumor proportion score (TPS) of about 50% or greater.

Exemplary Embodiment 254: The method of any one of embodiments 207-216 and 222-253, wherein the immunotherapy, the RB1-targeted therapy, the KRAS-targeted therapy, or the TP53-targeted therapy is a small molecule inhibitor, an antibody, a cellular therapy, or a nucleic acid.

Exemplary Embodiment 255: The method of embodiment 254, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 256: The method of embodiment 254, wherein the nucleic acid comprises a double-stranded RNA (dsRNA), a small interfering RNA (siRNA), or a small hairpin RNA (shRNA).

Exemplary Embodiment 257: The method of any one of embodiments 207-216 and 222-254, wherein:
(a) the RB1-targeted therapy comprises one or more of a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor;
(b) the KRAS-targeted therapy comprises one or more of a kinase inhibitor, a farnesyltransferase inhibitor, a geranylgeranyltransferase inhibitor, a palmitoylation inhibitor, an inhibitor of methylation cleavage, or an agent that inhibits the modification or post-translational processing of KRAS; or
(c) the TP53-targeted therapy comprises one or more of a p53 reactivator, an mTOR inhibitor, a Pin1 inhibitor, an ATR inhibitor, a proteasome inhibitor, a CHK inhibitor, an ATM inhibitor, a WEE1 inhibitor, or a murine double minute 2 (MDM2) inhibitor.

Exemplary Embodiment 258: The method of embodiment 254, wherein the immunotherapy comprises one or more of a checkpoint inhibitor, a cancer vaccine, a cell-based therapy, a T cell receptor (TCR)-based therapy, an adjuvant immunotherapy, a cytokine immunotherapy, or an oncolytic virus therapy.

Exemplary Embodiment 259: The method of any one of embodiments 239-256 and 258, wherein the KRAS-targeted therapy comprises a KRAS (G12C)-targeted therapy.

Exemplary Embodiment 260: The method of embodiment 259, wherein the KRAS (G12C)-targeted therapy comprises one or more of a KRAS inhibitor, a KRAS (G12C) inhibitor, and/or a SHP2 inhibitor.

Exemplary Embodiment 261: The method of any one of embodiments 163-260, wherein the NSCLC is advanced NSCLC and/or metastatic NSCLC.

Exemplary Embodiment 262: The method of any one of embodiments 163-261, wherein the NSCLC has an adenocarcinoma, squamous cell carcinoma, not otherwise specified, large cell neuroendocrine, sarcomatoid, or adenosquamous carcinoma subtype.

Exemplary Embodiment 263: The method of any one of embodiments 207-262, wherein the NSCLC comprises a TMB of about 9.4 mut/Mb.

Exemplary Embodiment 264: The method of any one of embodiments 163-175, 180-216, and 222-263, wherein the treatment or the one or more treatment options further comprise an additional anti-cancer treatment.

Exemplary Embodiment 265: The method of embodiment 264, wherein the additional anti-cancer treatment comprises one or more of a small molecule inhibitor, an antibody, a cellular therapy, or a nucleic acid.

Exemplary Embodiment 266: The method of embodiment 265, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 267: The method of embodiment 265, wherein the nucleic acid comprises a double-stranded RNA (dsRNA), a small interfering RNA (siRNA), or a small hairpin RNA (shRNA).

Exemplary Embodiment 268: The method of embodiment 264, wherein the additional anti-cancer treatment comprises one or more of a surgery, radiotherapy, chemotherapy, anti-angiogenic therapy, anti-DNA repair therapy, anti-inflammatory therapy, an anti-neoplastic agent, a chemotherapeutic agent, a growth inhibitory agent, an anti-angiogenic agent, or a cytotoxic agent.

Exemplary Embodiment 269: The method of any one of embodiments 163-180, 182-221, and 223-268, wherein the sample from the individual comprises fluid, cells, or tissue.

Exemplary Embodiment 270: The method of any one of embodiments 163-180, 182-221, and 223-269, wherein the sample from the individual comprises a tumor biopsy or a circulating tumor cell.

Exemplary Embodiment 271: The method of embodiment 269 or embodiment 270, wherein the sample from the individual comprises one or more nucleic acids.

Exemplary Embodiment 272: The method of embodiment 271, wherein the sample from the individual comprises mRNA, genomic DNA, circulating tumor DNA, cell-free DNA, or cell-free RNA.

Exemplary Embodiment 273: The method of embodiment 269 or embodiment 270, wherein the sample from the individual comprises one or more polypeptides.

Exemplary Embodiment 274: The method of any one of embodiments 163-180, 182-221, and 223-273, wherein the sample is a formalin-fixed paraffin-embedded (FFPE) sample.

Exemplary Embodiment 275: The method of any one of embodiments 163-180, 182-221, and 223-274, wherein the sample comprises one or more nucleic acids and/or one or more polypeptides obtained from an FFPE sample from the individual.

Exemplary Embodiment 276: The method of embodiment 275, wherein the one or more nucleic acids comprise mRNA and/or genomic DNA.

Exemplary Embodiment 277: The method of any one of embodiments 269-276, wherein the sample is derived from a NSCLC in the individual.

Exemplary Embodiment 278: The method of any one of embodiments 179-180, 186, 220, 229, 233, and 236, wherein the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample.

Exemplary Embodiment 279: The method of embodiment 278, wherein the bait comprises a capture nucleic acid molecule configured to hybridize to the one or more nucleic acids.

Exemplary Embodiment 280: The method of embodiment 279, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and 200 nucleotides.

Exemplary Embodiment 281: The method of any one of embodiments 278-280, wherein the bait is conjugated to an affinity reagent or to a detection reagent.

Exemplary Embodiment 282: The method of embodiment 281, wherein the affinity reagent is an antibody, an antibody fragment, or biotin, or wherein the detection reagent is a fluorescent marker.

Exemplary Embodiment 283: The method of any one of embodiments 279-282, wherein the capture nucleic acid molecule comprises a DNA, RNA, or mixed DNA/RNA molecule.

Exemplary Embodiment 284: The method of any one of embodiments 179-180, 186, 220, 229, 233, and 236, wherein the selectively enriching comprises amplifying the one or more nucleic acids in the sample using a polymerase chain reaction (PCR) to produce the enriched sample.

Exemplary Embodiment 285: The method of any one of embodiments 278-284, further comprising sequencing the one or more nucleic acid molecules in the enriched sample.

Exemplary Embodiment 286: A system, comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acids, wherein the one or more nucleic acids are derived from a sample obtained from an individual;
(b) analyze the plurality of sequence reads for the presence of a deletion of a methylthioadenosine phosphorylase (MTAP) gene, or of a portion thereof, and one or more mutations in an EGFR gene or in a SMARCA4 gene; and
(c) detect, based on the analyzing, a deletion of an MTAP gene, or of a portion thereof, and one or more mutations in an EGFR gene or in a SMARCA4 gene in the sample.

Exemplary Embodiment 287: The system of embodiment 286, wherein the sample is obtained from an individual having a non-small cell lung cancer (NSCLC).

Exemplary Embodiment 288: The system of embodiment 286 or embodiment 287, wherein the one or more mutations comprise one or more of an insertion, deletion or substitution of one or more nucleotides, a genomic rearrangement, an alteration in a promoter, a gene fusion, or a copy number alteration.

Exemplary Embodiment 289: The system of any one of embodiments 286-288, wherein the one or more mutations result in one or more of an insertion, deletion or substitution of one or more amino acid residues in a polypeptide encoded by the EGFR gene or the SMARCA4 gene.

Exemplary Embodiment 290: The system of any one of embodiments 286-289, wherein the plurality of sequence reads is obtained by sequencing, whole exome sequencing, whole genome sequencing, gene-targeted sequencing, or next-generation sequencing.

Exemplary Embodiment 291: A non-transitory computer readable storage medium comprising one or more programs executable by one or more computer processors for performing a method, comprising:
(a) obtaining, using the one or more processors, a plurality of sequence reads of one or more nucleic acids, wherein the one or more nucleic acids are derived from a sample obtained from an individual;
(b) analyzing, using the one or more processors, the plurality of sequence reads for the presence of a deletion of a methylthioadenosine phosphorylase (MTAP) gene, or of a portion thereof, and one or more mutations in an EGFR gene or in a SMARCA4 gene; and
(c) detecting, using the one or more processors and based on the analyzing, a deletion of an MTAP gene, or of a portion thereof, and one or more mutations in an EGFR gene or in a SMARCA4 gene, in the sample.

Exemplary Embodiment 292: The non-transitory computer readable storage medium of embodiment 291, wherein the sample is obtained from an individual having a non-small cell lung cancer (NSCLC).

Exemplary Embodiment 293: The non-transitory computer readable storage medium of embodiment 291 or embodiment 292, wherein the one or more mutations comprise one or more of an insertion, deletion or substitution of one or more nucleotides, a genomic rearrangement, an alteration in a promoter, a gene fusion, or a copy number alteration.

Exemplary Embodiment 294: The non-transitory computer readable storage medium of any one of embodiments 292-293, wherein the one or more mutations result in one or more of an insertion, deletion or substitution of one or more amino acid residues in a polypeptide encoded by the EGFR gene or the SMARCA4 gene.

Exemplary Embodiment 295: The non-transitory computer readable storage medium of any one of embodiments 292-294, wherein the plurality of sequence reads is obtained by sequencing, whole exome sequencing, whole genome sequencing, gene-targeted sequencing, or next-generation sequencing.

Exemplary Embodiment 296: A system, comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acids, wherein the one or more nucleic acids are derived from a sample obtained from an individual;
(b) analyze the plurality of sequence reads for the presence of a tumor mutational burden (TMB) of at least about 10 mut/Mb or at least about 20 mut/Mb, or one or more mutations in an RB1 gene, a KRAS gene, or a TP53 gene; and
(c) detect, based on the analyzing, a TMB of at least about 10 mut/Mb or at least about 20 mut/Mb, or one or more mutations in an RB1 gene, a KRAS gene, or a TP53 gene in the sample.

Exemplary Embodiment 297: The system of embodiment 296, wherein the sample is obtained from an individual having a non-small cell lung cancer (NSCLC).

Exemplary Embodiment 298: The system of embodiment 296 or embodiment 297, wherein the one or more mutations comprise one or more of an insertion, deletion or substitution of one or more nucleotides, a genomic rearrangement, an alteration in a promoter, a gene fusion, or a copy number alteration.

Exemplary Embodiment 299: The system of any one of embodiments 296-298, wherein the one or more mutations result in one or more of an insertion, deletion or substitution of one or more amino acid residues in a polypeptide encoded by the RB1 gene, KRAS gene, or TP53 gene.

Exemplary Embodiment 300: The system of any one of embodiments 296-299, wherein the analyzing the plurality of sequence reads for the presence of a TMB of at least about 10 mut/Mb or at least about 20 mut/Mb is based on about 0.80 Mb of sequenced DNA.

Exemplary Embodiment 301: The system of any one of embodiments 296-299, wherein the analyzing the plurality of sequence reads for the presence of a TMB of at least about 10 mut/Mb or at least about 20 mut/Mb is based on between about 0.83 Mb and about 1.14 Mb of sequenced DNA.

Exemplary Embodiment 302: The system of any one of embodiments 296-299, wherein the analyzing the plurality of sequence reads for the presence of a TMB of at least about 10 mut/Mb or at least about 20 mut/Mb is based on about 1.1 Mb of sequenced DNA.

Exemplary Embodiment 303: The system of any one of embodiments 296-299, wherein the analyzing the plurality of sequence reads for the presence of a TMB of at least about 10 mut/Mb or at least about 20 mut/Mb is based on up to about 1.1 Mb of sequenced DNA.

Exemplary Embodiment 304: The system of any one of embodiments 296-303, wherein the plurality of sequence reads is obtained by sequencing, whole exome sequencing, whole genome sequencing, gene-targeted sequencing, or next-generation sequencing.

Exemplary Embodiment 305: A non-transitory computer readable storage medium comprising one or more programs executable by one or more computer processors for performing a method, comprising:
(a) obtaining, using the one or more processors, a plurality of sequence reads of one or more nucleic acids, wherein the one or more nucleic acids are derived from a sample obtained from an individual;
(b) analyzing, using the one or more processors, the plurality of sequence reads for the presence of a tumor mutational burden (TMB) of at least about 10 mut/Mb or at least about 20 mut/Mb, or one or more mutations in an RB1 gene, a KRAS gene, or a TP53 gene; and
(c) detecting, using the one or more processors and based on the analyzing, TMB of at least about 10 mut/Mb or at least about 20 mut/Mb, or one or more mutations in an RB1 gene, a KRAS gene, or a TP53 gene, in the sample.

Exemplary Embodiment 306: The non-transitory computer readable storage medium of embodiment 305, wherein the sample is obtained from an individual having a non-small cell lung cancer (NSCLC).

Exemplary Embodiment 307: The non-transitory computer readable storage medium of embodiment 305 or embodiment 306, wherein the one or more mutations comprise one or more of an insertion, deletion or substitution of one or more nucleotides, a genomic rearrangement, an alteration in a promoter, a gene fusion, or a copy number alteration.

Exemplary Embodiment 308: The non-transitory computer readable storage medium of any one of embodiments 305-307, wherein the one or more mutations result in one or more of an insertion, deletion or substitution of one or more amino acid residues in a polypeptide encoded by the RB1 gene, KRAS gene, or TP53 gene.

Exemplary Embodiment 309: The non-transitory computer readable storage medium of any one of embodiments 305-308, wherein the analyzing the plurality of sequence reads for the presence of a tumor mutational burden (TMB) of at least about 10 mut/Mb or at least about 20 mut/Mb is based on about 0.80 Mb of sequenced DNA.

Exemplary Embodiment 310: The non-transitory computer readable storage medium of any one of embodiments 305-308, wherein the analyzing the plurality of sequence reads for the presence of a TMB of at least about 10 mut/Mb or at least about 20 mut/Mb is based on between about 0.83 Mb and about 1.14 Mb of sequenced DNA.

Exemplary Embodiment 311: The non-transitory computer readable storage medium of any one of embodiments 305-308, wherein the analyzing the plurality of sequence reads for the presence of a TMB of at least about 10 mut/Mb or at least about 20 mut/Mb is based on about 1.1 Mb of sequenced DNA.

Exemplary Embodiment 312: The non-transitory computer readable storage medium of any one of embodiments 305-308, wherein the analyzing the plurality of sequence reads for the presence of a TMB of at least about 10 mut/Mb or at least about 20 mut/Mb is based on up to about 1.1 Mb of sequenced DNA.

Exemplary Embodiment 313: The non-transitory computer readable storage medium of any one of embodiments 305-312, wherein the plurality of sequence reads is obtained by sequencing, whole exome sequencing, whole genome sequencing, gene-targeted sequencing, or next-generation sequencing.

Exemplary Embodiment 314: An EGFR-targeted therapy for use in a method of treating or delaying progression of a NSCLC, wherein the method comprises administering an EGFR-targeted therapy to an individual having a NSCLC, wherein a deletion of an MTAP gene, or of a portion thereof, and one or more mutations in an EGFR gene are detected in a sample obtained from the individual.

Exemplary Embodiment 315: A SMARCA4-targeted therapy for use in a method of treating or delaying progression of a NSCLC, wherein the method comprises administering a SMARCA4-targeted therapy to an individual having a NSCLC, wherein a deletion of an MTAP gene, or of a portion thereof, and one or more mutations in a SMARCA4 gene are detected in a sample obtained from the individual.

Exemplary Embodiment 316: An immunotherapy for use in a method of treating or delaying progression of a NSCLC, wherein the method comprises administering an immunotherapy to an individual having a NSCLC, wherein a TMB of at least about 10 mut/Mb or at least about 20 mut/Mb, and/or a PD-L1-positive NSCLC are detected in a sample obtained from the individual.

Exemplary Embodiment 317: The immunotherapy of embodiment 316, wherein the absence of a deletion of an MTAP gene, or of a portion thereof, is further detected in the sample.

Exemplary Embodiment 318: An RB1-targeted therapy for use in a method of treating or delaying progression of a NSCLC, wherein the method comprises administering an RB1-targeted therapy to an individual having a NSCLC, wherein one or more mutations in an RB1 gene are detected in a sample obtained from the individual.

Exemplary Embodiment 319: The RB1-targeted therapy of embodiment 318, wherein the absence of a deletion of an MTAP gene, or of a portion thereof, is further detected in the sample.

Exemplary Embodiment 320: A KRAS-targeted therapy for use in a method of treating or delaying progression of a NSCLC, wherein the method comprises administering a KRAS-targeted therapy to an individual having a NSCLC, wherein one or more mutations in a KRAS gene are detected in a sample obtained from the individual.

Exemplary Embodiment 321: The KRAS-targeted therapy of embodiment 320, wherein the absence of a deletion of an MTAP gene, or of a portion thereof, is further detected in the sample.

Exemplary Embodiment 322: A TP53-targeted therapy for use in a method of treating or delaying progression of a NSCLC, wherein the method comprises administering a TP53-targeted therapy to an individual having a NSCLC, wherein one or more mutations in a TP53 gene are detected in a sample obtained from the individual.

Exemplary Embodiment 323: The TP53-targeted therapy of embodiment 322, wherein the absence of a deletion of an MTAP gene, or of a portion thereof, is further detected in the sample.

Exemplary Embodiment 324: An EGFR-targeted therapy for use in the manufacture of a medicament for treating or delaying progression of a NSCLC, wherein the medicament is to be administered to an individual having a NSCLC, wherein a deletion of an MTAP gene, or of a portion thereof, and one or more mutations in an EGFR gene are detected in a sample obtained from the individual.

Exemplary Embodiment 325: A SMARCA4-targeted therapy for use in the manufacture of a medicament for treating or delaying progression of a NSCLC, wherein the medicament is to be administered to an individual having a NSCLC, wherein a deletion of an MTAP gene, or of a portion thereof, and one or more mutations in a SMARCA4 gene are detected in a sample obtained from the individual.

Exemplary Embodiment 326: An immunotherapy for use in the manufacture of a medicament for treating or delaying progression of a NSCLC, wherein the medicament is to be administered to an individual having a NSCLC, wherein a TMB of at least about 10 mut/Mb or at least about 20 mut/Mb, and/or a PD-L1-positive NSCLC are detected in a sample obtained from the individual.

Exemplary Embodiment 327: The immunotherapy of embodiment 326, wherein the absence of a deletion of an MTAP gene, or of a portion thereof, is further detected in the sample.

Exemplary Embodiment 328: An RB1-targeted therapy for use in the manufacture of a medicament for treating or delaying progression of a NSCLC, wherein the medicament is to be administered to an individual having a NSCLC, wherein one or more mutations in an RB1 gene are detected in a sample obtained from the individual.

Exemplary Embodiment 329: The RB1-targeted therapy of embodiment 328, wherein the absence of a deletion of an MTAP gene, or of a portion thereof, is further detected in the sample.

Exemplary Embodiment 330: A KRAS-targeted therapy for use in the manufacture of a medicament for treating or delaying progression of a NSCLC, wherein the medicament is to be administered to an individual having a NSCLC, wherein one or more mutations in a KRAS gene are detected in a sample obtained from the individual.

Exemplary Embodiment 331: The KRAS-targeted therapy of embodiment 330, wherein the absence of a deletion of an MTAP gene, or of a portion thereof, is further detected in the sample.

Exemplary Embodiment 332: A TP53-targeted therapy for use in the manufacture of a medicament for treating or delaying progression of a NSCLC, wherein the medicament is to be administered to an individual having a NSCLC, wherein one or more mutations in a TP53 gene are detected in a sample obtained from the individual.

Exemplary Embodiment 333: The TP53-targeted therapy of embodiment 332, wherein the absence of a deletion of an MTAP gene, or of a portion thereof, is further detected in the sample.

Exemplary Embodiment 334: A method of identifying an individual having a sarcomatoid renal cell carcinoma (srcRCC) or a clear cell renal cell carcinoma (ccRCC) who may benefit from a treatment comprising a neurofibromatosis type II (NF2)-targeted therapy, the method comprising detecting in a sample from the individual a deletion of a methylthioadenosine phosphorylase (MTAP) gene, or of a portion thereof, and one or more mutations in an NF2 gene, wherein the presence of the deletion of the MTAP gene, or of the portion thereof, and one or more mutations in the NF2 gene in the sample identifies the individual as one who may benefit from a treatment comprising an NF2-targeted therapy.

Exemplary Embodiment 335: A method of selecting a treatment for an individual having a srcRCC or a ccRCC, the method comprising detecting a deletion of an MTAP gene, or of a portion thereof, and one or more mutations in an NF2 gene in a sample from the individual, wherein the presence of the deletion of the MTAP gene, or of the portion thereof, and one or more mutations in the NF2 gene in the sample identifies the individual as one who may benefit from a treatment comprising an NF2-targeted therapy.

Exemplary Embodiment 336: The method of embodiment 334 or embodiment 335, wherein the presence in the sample of the deletion of the MTAP gene, or of the portion thereof, and the one or more mutations in the NF2 gene identifies the individual as one who may benefit from an additional treatment comprising a protein arginine methyltransferase 5 (PRMT5)-targeted therapy.

Exemplary Embodiment 337: A method of identifying one or more treatment options for an individual having a srcRCC or a ccRCC, the method comprising:
(a) detecting a deletion of an MTAP gene, or of a portion thereof, and one or more mutations in an NF2 gene in a sample from the individual; and
(b) generating a report, wherein the report comprises one or more treatment options identified for the individual based at least in part on the presence of the deletion of the MTAP gene, or of the portion thereof, and one or more mutations in the NF2 gene in the sample, wherein the one or more treatment options comprise an NF2-targeted therapy.

Exemplary Embodiment 338: A method of identifying one or more treatment options for an individual having a srcRCC or a ccRCC, the method comprising acquiring knowledge of a deletion of an MTAP gene, or of a portion thereof, and one or more mutations in an NF2 gene in a sample from the individual; and generating a report comprising one or more treatment options identified for the individual based at least in part on the knowledge of a deletion of the MTAP gene, or of a portion thereof, and one or more mutations in the NF2 gene in the sample, wherein the one or more treatment options comprise an NF2-targeted therapy.

Exemplary Embodiment 339: The method of embodiment 337 or embodiment 338, wherein the one or more treatment options identified for the individual further comprise a PRMT5-targeted therapy.

Exemplary Embodiment 340: A method of selecting a treatment for an individual having a srcRCC or a ccRCC, comprising acquiring knowledge of a deletion of an MTAP gene, or of a portion thereof, and one or more mutations in an NF2 gene in a sample from the individual, wherein responsive to the acquisition of knowledge of a deletion of an MTAP gene, or of a portion thereof, and one or more mutations in an NF2 gene in the sample: (i) the individual is classified as a candidate to receive a treatment comprising an NF2-targeted therapy; and/or (ii) the individual is identified as likely to respond to a treatment that comprises an NF2-targeted therapy.

Exemplary Embodiment 341: The method of embodiment 340, wherein the treatment further comprises a PRMT5-targeted therapy.

Exemplary Embodiment 342: A method of predicting survival of an individual having a srcRCC or a ccRCC treated with a treatment comprising an NF2-targeted therapy, the method comprising acquiring knowledge of a deletion of an MTAP gene, or of a portion thereof, and one or more mutations in an NF2 gene in a sample from the individual, wherein responsive to the acquisition of said knowledge, the individual is predicted to have longer survival after treatment with the NF2-targeted therapy, as compared to an individual whose srcRCC or ccRCC does not exhibit a deletion of an MTAP gene, or of a portion thereof, and one or more mutations in an NF2 gene.

Exemplary Embodiment 343: A method of treating or delaying progression of a srcRCC or a ccRCC, comprising acquiring knowledge of a deletion of an MTAP gene, or of a portion thereof, and one or more mutations in an NF2 gene in a sample from an individual, and responsive to said knowledge, administering to the individual an effective amount of a treatment comprising an NF2-targeted therapy.

Exemplary Embodiment 344: A method of treating or delaying progression of a srcRCC or a ccRCC, comprising responsive to acquiring knowledge of a deletion of an MTAP gene, or of a portion thereof, and one or more mutations in an NF2 gene in a sample from an individual, administering to the individual an effective amount of a treatment comprising an NF2-targeted therapy.

Exemplary Embodiment 345: The method of any one of embodiments 338-344, wherein the acquiring knowledge of the deletion of the MTAP gene, or of the portion thereof, and one or more mutations in the NF2 gene comprises detecting the deletion of the MTAP gene, or of the portion thereof, and the one or more mutations in the NF2 gene in the sample.

Exemplary Embodiment 346: A method of treating or delaying progression of a srcRCC or a ccRCC, comprising detecting a deletion of an MTAP gene, or of a portion thereof, and one or more mutations in an NF2 gene in a sample from an individual, and administering to the individual an effective amount of a treatment comprising an NF2-targeted therapy.

Exemplary Embodiment 347: A method of detecting the presence or absence of a srcRCC or a ccRCC in an individual, comprising:
(a) detecting the presence or absence of a srcRCC or a ccRCC in a sample from an individual; and
(b) detecting the presence or absence of a deletion of an MTAP gene, or of a portion thereof, and one or more mutations in an NF2 gene in the sample.

Exemplary Embodiment 348: A method of assessing a srcRCC or a ccRCC in an individual, the method comprising:
(a) detecting a deletion of an MTAP gene, or of a portion thereof, and one or more mutations in an NF2 gene in a sample from the individual; and
(b) providing an assessment of the deletion of the MTAP gene, or of the portion thereof, and the one or more mutations in the NF2 gene.

Exemplary Embodiment 349: A method of detecting a deletion of an MTAP gene, or of a portion thereof, and one or more mutations in an NF2 gene, the method comprising detecting the deletion of the MTAP gene, or of the portion thereof, and the one or more mutations in the NF2 gene in a sample from an individual having a srcRCC or a ccRCC.

Exemplary Embodiment 350: A method of detecting a deletion of an MTAP gene, or of a portion thereof, and one or more mutations in an NF2 gene, the method comprising:
(a) providing a sample from an individual having a srcRCC or a ccRCC, wherein the sample comprises one or more nucleic acids;
(b) preparing a nucleic acid sequencing library from the one or more nucleic acids in the sample;
(c) amplifying said library using a polymerase chain reaction (PCR);
(d) selectively enriching for one or more nucleic acids comprising MTAP or NF2 gene nucleotide sequences in said library to produce an enriched sample;
(e) sequencing the enriched sample, thereby producing a plurality of sequencing reads;
(f) analyzing the plurality of sequencing reads for the presence of a deletion of an MTAP gene, or of a portion thereof, and one or more mutations in an NF2 gene;
(g) detecting, based on the analyzing step, a deletion of an MTAP gene, or of a portion thereof, and one or more mutations in an NF2 gene in the sample from the individual.

Exemplary Embodiment 351: The method of any one of embodiments 334-337 and 345-350, further comprising selectively enriching for one or more of: (a) one or more nucleic acids comprising nucleotide sequences that comprise the deletion of the MTAP gene, or of the portion thereof, or (b) one or more nucleic acids comprising nucleotide sequences that comprise the one or more mutations in the NF2 gene; wherein the selectively enriching produces an enriched sample.

Exemplary Embodiment 352: A method of treating or delaying progression of a srcRCC or a ccRCC, comprising administering to an individual having a srcRCC or a ccRCC an effective amount of a treatment comprising an NF2-targeted therapy, wherein the srcRCC or ccRCC comprises a deletion of an MTAP gene, or of a portion thereof, and one or more mutations in an NF2 gene.

Exemplary Embodiment 353: The method of any one of embodiments 334-351, further comprising acquiring knowledge of or detecting in the sample a deletion of a CDKN2A gene, or of a portion thereof; and/or a deletion of a CDKN2B gene, or of a portion thereof.

Exemplary Embodiment 354: The method of any one of embodiments 334-351 and 353, comprising acquiring knowledge of or detecting in the sample one or more mutations in one or more genes, wherein the one or more genes comprise VHL, PBRM1, SETD2, BAP1, TP53, PTEN, TERT, TSC1, PIK3CA, ATM, or MET.

Exemplary Embodiment 355: The method of any one of embodiments 334-354, wherein the individual has one or more mutations in one or more genes, wherein the one or more genes comprise VHL, PBRM1, SETD2, BAP1, TP53, PTEN, TERT, TSC1, PIK3CA, ATM, or MET.

Exemplary Embodiment 356: The method of embodiment 354 or embodiment 355, further comprising administering to the individual an effective amount of a treatment comprising an anti-cancer therapy.

Exemplary Embodiment 357: The method of any one of embodiments 354-356, further comprising selectively enriching for one or more nucleic acids comprising nucleotide sequences that comprise one or more mutations in one or more genes, wherein the one or more genes comprise VHL, PBRM1, SETD2, BAP1, TP53, PTEN, TERT, TSC1, PIK3CA, ATM, or MET, to produce an enriched sample.

Exemplary Embodiment 358: The method of any one of embodiments 354-357, wherein the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 359: The method of any one of embodiments 334-358, wherein the individual has a deletion of a CDKN2A gene, or of a portion thereof; and/or a deletion of a CDKN2B gene, or of a portion thereof.

Exemplary Embodiment 360: The method of embodiment 359, wherein the individual has a heterozygous or a homozygous deletion of a CDKN2A gene, or of a portion thereof; and/or a heterozygous or a homozygous deletion of a CDKN2B gene, or of a portion thereof.

Exemplary Embodiment 361: The method of any one of embodiments 334-360, wherein the individual has a heterozygous or a homozygous deletion of the MTAP gene, or of a portion thereof.

Exemplary Embodiment 362: The method of any one of embodiments 343-346 and 351-361, further comprising administering to the individual a PRMT5-targeted therapy.

Exemplary Embodiment 363: The method of any one of embodiments 334-362, wherein the individual is being treated with a PRMT5-targeted therapy.

Exemplary Embodiment 364: The method of any one of embodiments 336, 339, 341, and 362-363, wherein the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

Exemplary Embodiment 365: The method of any one of embodiments 334-364, wherein the one or more mutations in the NF2 gene comprise one or more of an insertion, deletion or substitution of one or more nucleotides, a genomic rearrangement, an alteration in a promoter, a gene fusion, or a copy number alteration.

Exemplary Embodiment 366: The method of any one of embodiments 334-365, wherein the one or more mutations in the NF2 gene result in one or more of an insertion, deletion or substitution of one or more amino acid residues in a polypeptide encoded by the NF2 gene.

Exemplary Embodiment 367: The method of any one of embodiments 334-346 and 351-366, wherein the NF2-targeted therapy is a small molecule inhibitor, an antibody, a cellular therapy, or a nucleic acid.

Exemplary Embodiment 368: The method of embodiment 367, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 369: The method of embodiment 367, wherein the nucleic acid comprises a double-stranded RNA (dsRNA), a small interfering RNA (siRNA), or a small hairpin RNA (shRNA).

Exemplary Embodiment 370: The method of any one of embodiments 334-346 and 351-367, wherein the NF2-targeted therapy comprises one or more of an mTOR inhibitor, a VEGF inhibitor, a focal adhesion kinase (FAK) inhibitor, an EGFR inhibitor, a NEDD8-activating enzyme (NAE) inhibitor, a MET inhibitor, a MEK inhibitor, a SRC inhibitor, a JNK inhibitor, a CDK inhibitor, a WEE1, a CHK1 inhibitor, or a multi-targeted kinase inhibitor.

Exemplary Embodiment 371: The method of any one of embodiments 334-337, 345-351, and 353-370, wherein the deletion of the MTAP gene or of the portion thereof, or the one or more mutations in the NF2 gene are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 372: The method of any one of embodiments 334-337, 345-351, and 353-370, wherein the deletion of the MTAP gene or of the portion thereof, or the one or more mutations in the NF2 gene are detected in a polypeptide encoded by the MTAP gene, or the NF2 gene.

Exemplary Embodiment 373: The method of embodiment 372, wherein the deletion of the MTAP gene or of the portion thereof, or the one or more mutations in the NF2 gene are detected in the sample by one or more of: immunoblotting, enzyme linked immunosorbent assay (ELISA), immunohistochemistry, or mass spectrometry.

Exemplary Embodiment 374: The method of any one of embodiments 353-373, wherein the deletion of the CDKN2A gene or of the portion thereof, and/or the deletion of the CDKN2B gene or of the portion thereof, are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 375: The method of any one of embodiments 353-373, wherein the deletion of the CDKN2A gene or of the portion thereof, and/or the deletion of the CDKN2B gene or of the portion thereof, are detected in a polypeptide encoded by the CDKN2A gene or the CDKN2B gene.

Exemplary Embodiment 376: The method of embodiment 375, wherein the deletion of the CDKN2A gene or of the portion thereof, and/or the deletion of the CDKN2B gene or of the portion thereof, are detected in the sample by one or more of: immunoblotting, enzyme linked immunosorbent assay (ELISA), immunohistochemistry, or mass spectrometry.

Exemplary Embodiment 377: A method of identifying an individual having a clear cell renal cell carcinoma (ccRCC) who may benefit from a treatment comprising a Von Hippel-Lindau tumor suppressor (VHL)-targeted therapy, or a protein polybromo-1 (PBRM1)-targeted therapy, the method comprising detecting in a sample from the individual one or more mutations in a VHL gene or a PBRM1 gene; wherein:
(i) the presence in the sample of one or more mutations in the VHL gene identifies the individual as one who may benefit from a treatment comprising a VHL-targeted therapy; or
(ii) the presence in the sample of one or more mutations in the PBRM1 gene identifies the individual as one who may benefit from a treatment comprising a PBRM1-targeted therapy.

Exemplary Embodiment 378: A method of selecting a treatment for an individual having a ccRCC, the method comprising detecting one or more mutations in a VHL gene or a PBRM1 gene, in a sample from the individual; wherein:
(i) the presence in the sample of one or more mutations in the VHL gene identifies the individual as one who may benefit from a treatment comprising a VHL-targeted therapy; or
(ii) the presence in the sample of one or more mutations in the PBRM1 gene identifies the individual as one who may benefit from a treatment comprising a PBRM1-targeted therapy.

Exemplary Embodiment 379: A method of identifying one or more treatment options for an individual having a ccRCC, the method comprising:
(a) detecting in a sample from the individual one or more mutations in a VHL gene or a PBRM1 gene; and
(b) generating a report, wherein the report comprises:
(i) one or more treatment options identified for the individual based at least in part on the presence of one or more mutations in the VHL gene in the sample, wherein the one or more treatment options comprise a VHL-targeted therapy;
(ii) one or more treatment options identified for the individual based at least in part on the presence of one or more mutations in the PBRM1 gene in the sample, wherein the one or more treatment options comprise a PBRM1-targeted therapy.

Exemplary Embodiment 380: A method of identifying one or more treatment options for an individual having a ccRCC, the method comprising acquiring knowledge of one or more mutations in a VHL gene or a PBRM1 gene, in a sample from the individual; and generating a report comprising:
(i) one or more treatment options identified for the individual based at least in part on the knowledge of one or more mutations in the VHL gene in the sample, wherein the one or more treatment options comprise a VHL-targeted therapy; or
(ii) one or more treatment options identified for the individual based at least in part on the knowledge of one or more mutations in the PBRM1 gene in the sample, wherein the one or more treatment options comprise a PBRM1-targeted therapy.

Exemplary Embodiment 381: A method of selecting a treatment for an individual having a ccRCC, comprising acquiring knowledge of one or more mutations in a VHL gene or a PBRM1 gene, in a sample from the individual, wherein:
(a) responsive to acquisition of knowledge of one or more mutations in the VHL gene in the sample: (i) the individual is classified as a candidate to receive a treatment comprising a VHL-targeted therapy; and/or (ii) the individual is identified as likely to respond to a treatment that comprises a VHL-targeted therapy; or
(b) responsive to acquisition of knowledge of one or more mutations in the PBRM1 gene in the sample: (i) the individual is classified as a candidate to receive a treatment comprising a PBRM1-targeted therapy; and/or (ii) the individual is identified as likely to respond to a treatment that comprises a PBRM1-targeted therapy.

Exemplary Embodiment 382: A method of predicting survival of an individual having a ccRCC treated with a treatment comprising a VHL-targeted therapy or a PBRM1-targeted therapy, the method comprising acquiring knowledge of one or more mutations in a VHL gene or a PBRM1 gene, in a sample from the individual; wherein responsive to acquisition of said knowledge, the individual is predicted to have longer survival after treatment with the VHL-targeted therapy or PBRM1-targeted therapy, as compared to an individual whose ccRCC does not exhibit one or more mutations in the VHL gene or PBRM1 gene.

Exemplary Embodiment 383: A method of treating or delaying progression of a ccRCC, comprising:
(a) acquiring knowledge of one or more mutations in a VHL gene in a sample from an individual, and responsive to said knowledge, administering to the individual an effective amount of a treatment comprising a VHL-targeted therapy; or
(b) acquiring knowledge of one or more mutations in a PBRM1 gene in a sample from an individual, and responsive to said knowledge, administering to the individual an effective amount of a treatment comprising a PBRM1-targeted therapy.

Exemplary Embodiment 384: A method of treating or delaying progression of a ccRCC, comprising:
(a) responsive to acquiring knowledge of one or more mutations in a VHL gene in a sample from an individual, administering to the individual an effective amount of a treatment comprising a VHL-targeted therapy; or
(b) responsive to acquiring knowledge of one or more mutations in a PBRM1 gene in a sample from an individual, administering to the individual an effective amount of a treatment comprising a PBRM1-targeted therapy.

Exemplary Embodiment 385: The method of any one of embodiments 380-384, wherein acquiring knowledge of one or more mutations in a VHL gene or a PBRM1 gene comprises detecting the one or more mutations in the VHL gene or PBRM1 gene in the sample.

Exemplary Embodiment 386: A method of treating or delaying progression of a ccRCC, comprising:
(a) detecting one or more mutations in a VHL gene in a sample from an individual, and administering to the individual an effective amount of a treatment comprising a VHL-targeted therapy; or
(b) detecting one or more mutations in a PBRM1 gene in a sample from an individual, and administering to the individual an effective amount of a treatment comprising a PBRM1-targeted therapy.

Exemplary Embodiment 387: A method of detecting the presence or absence of a ccRCC in an individual, comprising:
(a) detecting the presence or absence of a ccRCC in a sample from an individual; and
(i) detecting the presence or absence of one or more mutations in a VHL gene in the sample, or
(ii) detecting the presence or absence of one or more mutations in a PBRM1 gene in the sample.

Exemplary Embodiment 388: A method of assessing a ccRCC in an individual, the method comprising:
(a) detecting one or more mutations in a VHL gene or a PBRM1 gene, in a sample from the individual; and
(b) providing an assessment of the one or more mutations in the VHL gene or PBRM1 gene in the sample.

Exemplary Embodiment 389: A method of detecting one or more mutations in a VHL gene or a PBRM1 gene, the method comprising detecting the one or more mutations in the VHL gene or PBRM1 gene in a sample from an individual having a ccRCC.

Exemplary Embodiment 390: A method of detecting one or more mutations in a VHL gene or a PBRM1 gene, the method comprising:
(a) providing a sample from an individual having a ccRCC, wherein the sample comprises one or more nucleic acids;
(b) preparing a nucleic acid sequencing library from the one or more nucleic acids in the sample;
(c) amplifying said library using a polymerase chain reaction (PCR);
(d) selectively enriching for one or more nucleic acids comprising VHL gene or PBRM1 gene nucleotide sequences in said library to produce an enriched sample;
(e) sequencing the enriched sample, thereby producing a plurality of sequencing reads;
(f) analyzing the plurality of sequencing reads for the presence of one or more mutations in a VHL gene or a PBRM1 gene;
(g) detecting, based on the analyzing step, one or more mutations in a VHL gene or a PBRM1 gene in the sample from the individual.

Exemplary Embodiment 391: A method of treating or delaying progression of a ccRCC, comprising:
(a) administering to an individual having a ccRCC an effective amount of a treatment comprising a VHL-targeted therapy, wherein the ccRCC comprises one or more mutations in a VHL gene; or
(b) administering to an individual having a ccRCC an effective amount of a treatment comprising a PBRM1-targeted therapy, wherein the ccRCC comprises one or more mutations in a PBRM1 gene.

Exemplary Embodiment 392: The method of any one of embodiments 377-390, further comprising acquiring knowledge of the absence of a deletion of an MTAP gene, or of a portion thereof, in the sample.

Exemplary Embodiment 393: The method of embodiment 392, wherein acquiring knowledge of the absence of a deletion of an MTAP gene, or of a portion thereof, comprises detecting the absence of a deletion of an MTAP gene, or of a portion thereof, in the sample.

Exemplary Embodiment 394: The method of any one of embodiments 377-390 and 392-393, further comprising detecting the absence of a deletion of an MTAP gene, or of a portion thereof, in the sample.

Exemplary Embodiment 395: The method of any one of embodiments 377-390 and 392-394, comprising acquiring knowledge of or detecting in the sample one or more mutations in one or more genes, wherein the one or more genes comprise SETD2, BAP1, TP53, PTEN, TERT, TSC1, PIK3CA, ATM, NF2, or MET.

Exemplary Embodiment 396: The method of any one of embodiments 377-395, wherein the individual has one or more mutations in one or more genes, wherein the one or more genes comprise SETD2, BAP1, TP53, PTEN, TERT, TSC1, PIK3CA, ATM, NF2, or MET.

Exemplary Embodiment 397: The method of embodiment 395 or embodiment 396, further comprising administering to the individual an effective amount of a treatment comprising anti-cancer therapy.

Exemplary Embodiment 398: The method of embodiment 395 or embodiment 396, comprising selectively enriching for one or more nucleic acids comprising nucleotide sequences that comprise one or more mutations in one or more genes to produce an enriched sample, wherein the one or more genes comprise SETD2, BAP1, TP53, PTEN, TERT, TSC1, PIK3CA, ATM, NF2, or MET.

Exemplary Embodiment 399: The method of any one of embodiments 395-398, wherein the one or more mutations in the one or more genes are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 400: The method of embodiment 395 or embodiment 396, wherein the one or more mutations in the one or more genes are detected in a polypeptide encoded by the one or more genes.

Exemplary Embodiment 401: The method of embodiment 400, wherein the one or more mutations in the one or more genes are detected in the sample by one or more of immunoblotting, enzyme linked immunosorbent assay (ELISA), immunohistochemistry, or mass spectrometry.

Exemplary Embodiment 402: The method of embodiment 393 or embodiment 394, further comprising selectively enriching for one or more nucleic acids comprising MTAP nucleotide sequences to produce an enriched sample.

Exemplary Embodiment 403: The method of any one of embodiments 393-402, wherein the absence of a deletion of an MTAP gene, or of a portion thereof, is detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 404: The method of any one of embodiments 377-403, wherein the individual does not have a deletion of an MTAP gene, or of a portion thereof.

Exemplary Embodiment 405: The method of any one of embodiments 377-379, 385-390, and 392-404, further comprising selectively enriching for one or more nucleic acids comprising nucleotide sequences that comprise the one or more mutations in the VHL gene or PBRM1 gene to produce an enriched sample.

Exemplary Embodiment 406: The method of any one of embodiments 377-405, wherein the one or more mutations in the VHL gene or PBRM1 gene comprise one or more of an insertion, deletion or substitution of one or more nucleotides, a genomic rearrangement, an alteration in a promoter of the VHL gene or PBRM1 gene, a gene fusion, or a copy number alteration.

Exemplary Embodiment 407: The method of any one of embodiments 377-406, wherein the one or more mutations in the VHL gene or PBRM1 gene result in one or more of an insertion, deletion or substitution of one or more amino acid residues in a polypeptide encoded by the VHL gene or PBRM1 gene.

Exemplary Embodiment 408: The method of any one of embodiments 377-379, 385-390, and 392-407, wherein the one or more mutations in the VHL gene or PBRM1 gene are detected in the sample by one or more of: an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 409: The method of any one of embodiments 377-379, 385-390, and 392-407, wherein the one or more mutations in the VHL gene or PBRM1 gene are detected in a polypeptide encoded by the VHL gene or PBRM1 gene.

Exemplary Embodiment 410: The method of embodiment 409, wherein the one or more mutations in the VHL gene or PBRM1 gene are detected in the sample by one or more of immunoblotting, enzyme linked immunosorbent assay (ELISA), immunohistochemistry, or mass spectrometry.

Exemplary Embodiment 411: The method of any one of embodiments 377-386 and 391-410, wherein the VHL-targeted therapy or the PBRM1-targeted therapy is a small molecule inhibitor, an antibody, a cellular therapy, or a nucleic acid.

Exemplary Embodiment 412: The method of embodiment 411, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 413: The method of embodiment 411, wherein the nucleic acid comprises a double-stranded RNA (dsRNA), a small interfering RNA (siRNA), or a small hairpin RNA (shRNA).

Exemplary Embodiment 414: The method of any one of embodiments 377-386 and 391-411, wherein:
(a) the VHL-targeted therapy comprises one or more of a poly(ADP-ribose) polymerase (PARP) inhibitor, a glutaminase 1 (GLS-1) inhibitor, a VEGF inhibitor, a HIF-2alpha inhibitor, an HDAC inhibitor, a CDK4/6 inhibitor, a Tank binding kinase 1 (TBK1) inhibitor, an EZH1 and/or EZH2 inhibitor, a Rho-Associated Kinase 1 (ROCK1) inhibitor, a glucose transporter 1 (GLUT1) inhibitor, an autophagy modulator, or an immune checkpoint inhibitor; or
(b) the PBRM1-targeted therapy comprises one or more of an EZH2 inhibitor, a VEGF inhibitor, or an mTOR inhibitor.

Exemplary Embodiment 415: The method of any one of embodiments 334-376, wherein the srcRCC or ccRCC is advanced stage srcRCC or ccRCC and/or refractory srcRCC or ccRCC.

Exemplary Embodiment 416: The method of any one of embodiments 377-414, wherein the ccRCC is advanced stage ccRCC and/or refractory ccRCC.

Exemplary Embodiment 417: The method of any one of embodiments 334-376 and 415, wherein the srcRCC or ccRCC is microsatellite stable and/or has a low tumor mutational burden (TMB).

Exemplary Embodiment 418: The method of any one of embodiments 377-414 and 416, wherein the ccRCC is microsatellite stable and/or has a low tumor mutational burden (TMB).

Exemplary Embodiment 419: The method of any one of embodiments 334-346, 351-386, and 391-418, wherein the treatment or the one or more treatment options further comprise an additional anti-cancer treatment.

Exemplary Embodiment 420: The method of embodiment 419, wherein the additional anti-cancer treatment comprises one or more of a small molecule inhibitor, an antibody, a cellular therapy, or a nucleic acid.

Exemplary Embodiment 421: The method of embodiment 420, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 422: The method of embodiment 420, wherein the nucleic acid comprises a double-stranded RNA (dsRNA), a small interfering RNA (siRNA), or a small hairpin RNA (shRNA).

Exemplary Embodiment 423: The method of embodiment 419, wherein the additional anti-cancer treatment comprises one or more of a surgery, radiotherapy, chemotherapy, anti-angiogenic therapy, anti-DNA repair therapy, anti-inflammatory therapy, an anti-neoplastic agent, a chemotherapeutic agent, a growth inhibitory agent, an anti-angiogenic agent, or a cytotoxic agent.

Exemplary Embodiment 424: The method of any one of embodiments 334-351, 353-390, and 392-423, wherein the sample from the individual comprises fluid, cells, or tissue.

Exemplary Embodiment 425: The method of any one of embodiments 334-351, 353-390, and 392-424, wherein the sample from the individual comprises a tumor biopsy or a circulating tumor cell.

Exemplary Embodiment 426: The method of embodiment 424 or embodiment 425, wherein the sample from the individual comprises one or more nucleic acids.

Exemplary Embodiment 427: The method of embodiment 426, wherein the sample from the individual comprises mRNA, genomic DNA, circulating tumor DNA, cell-free DNA, or cell-free RNA.

Exemplary Embodiment 428: The method of embodiment 424 or embodiment 425, wherein the sample from the individual comprises one or more polypeptides.

Exemplary Embodiment 429: The method of any one of embodiments 334-351, 353-390, and 392-428, wherein the sample is a formalin-fixed paraffin-embedded (FFPE) sample.

Exemplary Embodiment 430: The method of any one of embodiments 334-351, 353-390, and 392-429, wherein the sample comprises one or more nucleic acids and/or one or more polypeptides obtained from an FFPE sample from the individual.

Exemplary Embodiment 431: The method of embodiment 430, wherein the one or more nucleic acids comprise mRNA and/or genomic DNA.

Exemplary Embodiment 432: The method of any one of embodiments 334-351, 353-390, and 392-431, wherein the sample is derived from a srcRCC or a ccRCC in the individual.

Exemplary Embodiment 433: The method of any one of embodiments 350-351, 357, 390, 398, 402, and 405, wherein the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample.

Exemplary Embodiment 434: The method of embodiment 433, wherein the bait comprises a capture nucleic acid molecule configured to hybridize to the one or more nucleic acids.

Exemplary Embodiment 435: The method of embodiment 434, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and 200 nucleotides.

Exemplary Embodiment 436: The method of any one of embodiments 433-435, wherein the bait is conjugated to an affinity reagent or to a detection reagent.

Exemplary Embodiment 437: The method of embodiment 436, wherein the affinity reagent is an antibody, an antibody fragment, or biotin, or wherein the detection reagent is a fluorescent marker.

Exemplary Embodiment 438: The method of any one of embodiments 434-437, wherein the capture nucleic acid molecule comprises a DNA, RNA, or mixed DNA/RNA molecule.

Exemplary Embodiment 439: The method of any one of embodiments 350-351, 357, 390, 398, 402, and 405, wherein the selectively enriching comprises amplifying the one or more nucleic acids in the sample using a polymerase chain reaction (PCR) to produce the enriched sample.

Exemplary Embodiment 440: The method of any one of embodiments 433-439, further comprising sequencing the one or more nucleic acid molecules in the enriched sample.

Exemplary Embodiment 441: A system comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acids, wherein the one or more nucleic acids are derived from a sample obtained from an individual;
(b) analyze the plurality of sequence reads for the presence of a deletion of a methylthioadenosine phosphorylase (MTAP) gene, or of a portion thereof, and one or more mutations in an NF2 gene; and
(c) detect, based on the analyzing, a deletion of an MTAP gene, or of a portion thereof, and one or more mutations in an NF2 gene in the sample.

Exemplary Embodiment 442: The system of embodiment 441, wherein the sample is obtained from an individual having a srcRCC or a ccRCC.

Exemplary Embodiment 443: The system of embodiment 441 or embodiment 442, wherein the one or more mutations comprise one or more of an insertion, deletion or substitution of one or more nucleotides, a genomic rearrangement, an alteration in a promoter, a gene fusion, or a copy number alteration.

Exemplary Embodiment 444: The system of any one of embodiments 441-443, wherein the one or more mutations in the NF2 gene result in one or more of an insertion, deletion or substitution of one or more amino acid residues in a polypeptide encoded by the NF2 gene.

Exemplary Embodiment 445: The system of any one of embodiments 441-444, wherein the plurality of sequence reads is obtained by sequencing, whole exome sequencing, whole genome sequencing, gene-targeted sequencing, or next-generation sequencing.

Exemplary Embodiment 446: A non-transitory computer readable storage medium comprising one or more programs executable by one or more computer processors for performing a method, comprising:
(a) obtaining, using the one or more processors, a plurality of sequence reads of one or more nucleic acids, wherein the one or more nucleic acids are derived from a sample obtained from an individual;
(b) analyzing, using the one or more processors, the plurality of sequence reads for the presence of a deletion of a methylthioadenosine phosphorylase (MTAP) gene, or of a portion thereof, and one or more mutations in an NF2 gene; and
(c) detecting, using the one or more processors and based on the analyzing, a deletion of an MTAP gene, or of a portion thereof, and one or more mutations in an NF2 gene, in the sample.

Exemplary Embodiment 447: The non-transitory computer readable storage medium of embodiment 446, wherein the sample is obtained from an individual having a srcRCC or a ccRCC.

Exemplary Embodiment 448: The non-transitory computer readable storage medium of embodiment 446 or embodiment 447, wherein the one or more mutations comprise one or more of an insertion, deletion or substitution of one or more nucleotides, a genomic rearrangement, an alteration in a promoter, a gene fusion, or a copy number alteration.

Exemplary Embodiment 449: The non-transitory computer readable storage medium of any one of embodiments 446-448, wherein the one or more mutations in the NF2 gene result in one or more of an insertion, deletion or substitution of one or more amino acid residues in a polypeptide encoded by the NF2 gene.

Exemplary Embodiment 450: The non-transitory computer readable storage medium of any one of embodiments 446-449, wherein the plurality of sequence reads is obtained by sequencing, whole exome sequencing, whole genome sequencing, gene-targeted sequencing, or next-generation sequencing.

Exemplary Embodiment 451: A system comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acids, wherein the one or more nucleic acids are derived from a sample obtained from an individual;
(b) analyze the plurality of sequence reads for the presence of one or more mutations in a VHL gene or a PBRM1 gene; and
(c) detect, based on the analyzing, one or more mutations in a VHL gene or a PBRM1 gene in the sample.

Exemplary Embodiment 452: The system of embodiment 451, wherein the sample is obtained from an individual having a ccRCC.

Exemplary Embodiment 453: The system of embodiment 451 or embodiment 452, wherein the one or more mutations comprise one or more of an insertion, deletion or substitution of one or more nucleotides, a genomic rearrangement, an alteration in a promoter, a gene fusion, or a copy number alteration.

Exemplary Embodiment 454: The system of any one of embodiments 451-453, wherein the one or more mutations in the VHL gene or PBRM1 gene result in one or more of an insertion, deletion or substitution of one or more amino acid residues in a polypeptide encoded by the VHL gene or PBRM1 gene.

Exemplary Embodiment 455: The system of any one of embodiments 451-454, wherein the plurality of sequence reads is obtained by sequencing, whole exome sequencing, whole genome sequencing, gene-targeted sequencing, or next-generation sequencing.

Exemplary Embodiment 456: A non-transitory computer readable storage medium comprising one or more programs executable by one or more computer processors for performing a method, comprising:
(a) obtaining, using the one or more processors, a plurality of sequence reads of one or more nucleic acids, wherein the one or more nucleic acids are derived from a sample obtained from an individual;
(b) analyzing, using the one or more processors, the plurality of sequence reads for the presence of one or more mutations in a VHL gene or a PBRM1 gene; and
(c) detecting, using the one or more processors and based on the analyzing, one or more mutations in a VHL gene or a PBRM1 gene, in the sample.

Exemplary Embodiment 457: The non-transitory computer readable storage medium of embodiment 456, wherein the sample is obtained from an individual having a ccRCC.

Exemplary Embodiment 458: The non-transitory computer readable storage medium of embodiment 456 or embodiment 457, wherein the one or more mutations comprise one or more of an insertion, deletion or substitution of one or more nucleotides, a genomic rearrangement, an alteration in a promoter, a gene fusion, or a copy number alteration.

Exemplary Embodiment 459: The non-transitory computer readable storage medium of any one of embodiments 456-458, wherein the one or more mutations in the VHL gene or PBRM1 gene result in one or more of an insertion, deletion or substitution of one or more amino acid residues in a polypeptide encoded by the VHL gene or PBRM1 gene.

Exemplary Embodiment 460: The non-transitory computer readable storage medium of any one of embodiments 456-459, wherein the plurality of sequence reads is obtained by sequencing, whole exome sequencing, whole genome sequencing, gene-targeted sequencing, or next-generation sequencing.

Exemplary Embodiment 461: An NF2-targeted therapy for use in a method of treating or delaying progression of a srcRCC or a ccRCC, wherein the method comprises administering an NF2-targeted therapy to an individual having a srcRCC or a ccRCC, wherein a deletion of an MTAP gene, or of a portion thereof, and one or more mutations in an NF2 gene are detected in a sample obtained from the individual.

Exemplary Embodiment 462: An VHL-targeted therapy for use in a method of treating or delaying progression of a ccRCC, wherein the method comprises administering a VHL-targeted therapy to an individual having a ccRCC, wherein one or more mutations in a VHL gene are detected in a sample obtained from the individual.

Exemplary Embodiment 463: The VHL-targeted therapy of embodiment 462, wherein the absence of a deletion of an MTAP gene, or of a portion thereof, is further detected in the sample.

Exemplary Embodiment 464: A PBRM1-targeted therapy for use in a method of treating or delaying progression of a ccRCC, wherein the method comprises administering a PBRM1-targeted therapy to an individual having a ccRCC, wherein one or more mutations in a PBRM1 gene are detected in a sample obtained from the individual.

Exemplary Embodiment 465: The PBRM1-targeted therapy of embodiment 464, wherein the absence of a deletion of an MTAP gene, or of a portion thereof, is further detected in the sample.

Exemplary Embodiment 466: An NF2-targeted therapy for use in the manufacture of a medicament for treating or delaying progression of a srcRCC or a ccRCC, wherein the medicament is to be administered to an individual having a srcRCC or a ccRCC, wherein a deletion of an MTAP gene, or of a portion thereof, and one or more mutations in an NF2 gene are detected in a sample obtained from the individual.

Exemplary Embodiment 467: A VHL-targeted therapy for use in the manufacture of a medicament for treating or delaying progression of a ccRCC, wherein the medicament is to be administered to an individual having a ccRCC, wherein one or more mutations in a VHL gene are detected in a sample obtained from the individual.

Exemplary Embodiment 468: The VHL-targeted therapy of embodiment 467, wherein the absence of a deletion of an MTAP gene, or of a portion thereof, is further detected in the sample.

Exemplary Embodiment 469: A PBRM1-targeted therapy for use in the manufacture of a medicament for treating or delaying progression of a ccRCC, wherein the medicament is to be administered to an individual having a ccRCC, wherein one or more mutations in a PBRM1 gene are detected in a sample obtained from the individual.

Exemplary Embodiment 470: The PBRM1-targeted therapy of embodiment 469, wherein the absence of a deletion of an MTAP gene, or of a portion thereof, is further detected in the sample.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Anti-Cancer Drug Targets in Urothelial Bladder Cancer Based on Methylthioadenosine Phosphorylase (MTAP) Genomic Loss

Identification of new therapeutic targets is paramount when urothelial bladder cancer (UCB) presents or progresses to chemorefractory metastatic disease. In this Example, comprehensive genomic profiling (CGP) was used to evaluate the incidence of UCB patients with a candidate synthetic lethality (e.g., loss of MTAP), and whether this incidence was correlated with treatment-guiding biomarkers.

### Methods

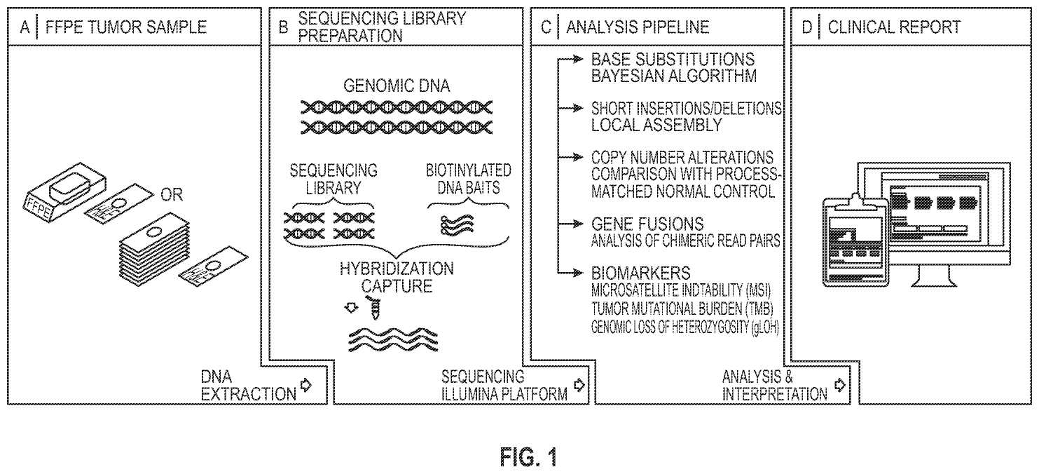

A U.S. Food and Drug Administration (FDA)-approved CGP assay (see, e.g., Frampton, et al. (2013) Nat Biotechnol 31(11):1023-31) was used to sequence 2,683 cases of clinically advanced UCB (FIG. 1). ≥50 ng of DNA were extracted from 40 μm of formalin-fixed paraffin-embedded (FFPE) sections. The workflow included hybrid capture-based sequencing using adaptor ligation-based libraries. Sequencing was performed with a mean coverage depth of >600×. Base substitutions, short variants, rearrangements, and copy number changes were assessed. Tumor mutational burden (TMB) was determined on 0.8 Mb of sequenced DNA, and microsatellite instability (MSI) was determined on 95 loci (see, e.g., Trabucco et al., J Mol Diagn (2019) 21(6):1053-1066). PD-L1 expression was determined by immunohistochemistry (IHC; Dako 22C3). PD-L1 negative was defined as 0% PD-L1 expression on tumor cells; PD-L1 low positive was defined as 1%-49% PD-L1 expression on tumor cells; and PD-L1 high positive was defined as >50% PD-L1 expression on tumor cells.

### Results

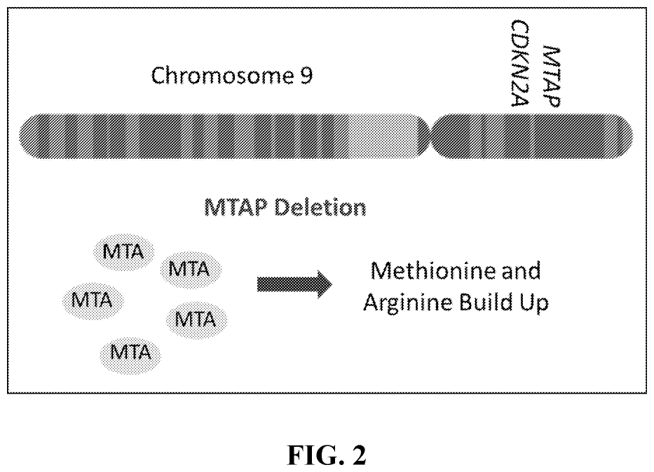

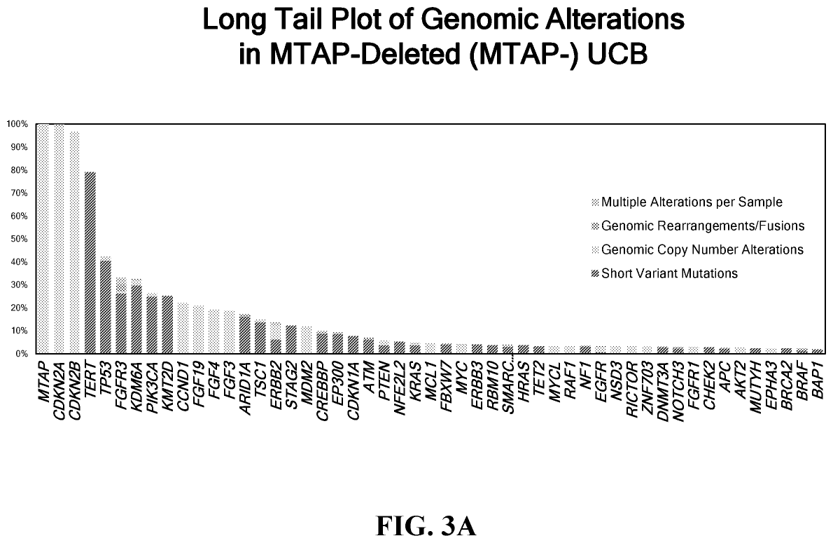

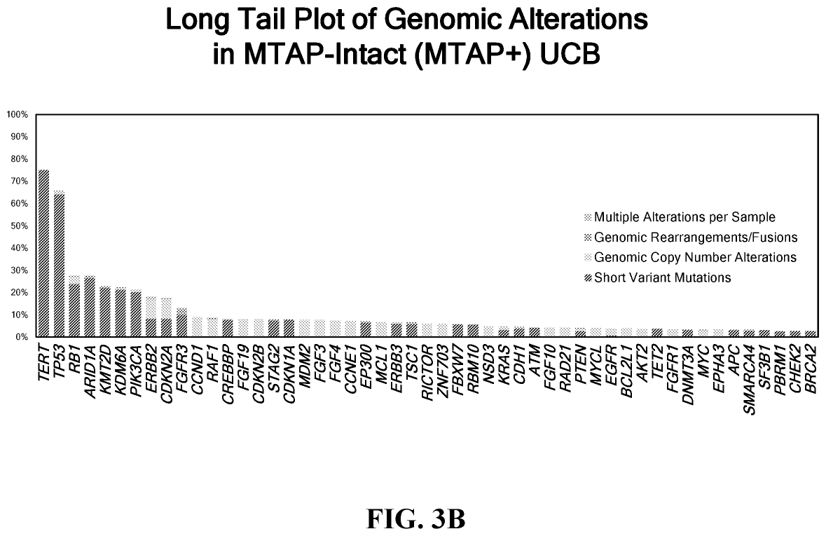

As shown in Table 1, 650 (24%) of analyzed UCB samples featured MTAP loss. The gene and age distributions were similar in MTAP intact (MTAP+) and MTAP-deleted (MTAP-) UCB. The number of genomic alterations per tumor was higher in MTAP-UCB, likely reflecting the significant genomic alteration co-deletions of CDKN2A/B at the 9p21 locus (FIG. 2). Of potential therapeutic targets, FGFR3 and PTEN genomic alterations were more frequent in MTAP-UCB (FIG. 3A) than in MTAP+ UCB (FIG. 3B). In contrast, biomarkers of immunotherapy (IO), such as high TMB and high PD-L1 IHC staining, were found with higher frequencies in MTAP+ UCB (Table 1).

TABLE 1
Results of comprehensive genomic profiling
(CGP) of MTAP-intact and MTAP-deleted UCB.

| | MTAP Intact | MTAP Loss | p Value* |
|---|---|---|---|
| Number of Cases | 2,033 | 650 | |
| Males/Females | 77%/23% | 72%/28% | NS |
| Median age (range) (years) | 70 (16-95) | 71 (31-98) | NS |
| Genomic alterations/tumor | 7.4 | 9.9 | |
| CDKN2A | 18% | 99.8% | <0.0001 |
| CDKN2B | 8% | 96% | <0.0001 |
| TP53 | 66% | 42% | <0.0001 |
| TERT | 75% | 79% | NS |
| FGFR3 | 13% | 33% | <0.0001 |
| PIK3CA | 21% | 26% | NS |
| ERBB2 | 18% | 14% | NS |
| EGFR | 4% | 4% | NS |
| PTEN | 4% | 6% | 0.04 |
| TSC1 | 7% | 2% | <0.0001 |
| BRCA1/2 | 2%/0% | 1%/2% | NS |
| MSI High | 1% | 1% | NS |
| Median TMB | 7.5 | 6.3 | |
| Mean TMB | 11.2 | 8.5 | <0.0001 |
| TMB ≥10 mutations/Mb | 41% | 30% | <0.0001 |
| TMB ≥20 mutations/Mb | 15% | 7% | <0.0001 |
| PD-L1 Low Positive | 20% | 24% | NS |
| PD-L1 High Positive | 22% | 3% | <0.0001 |

*NS, Not significant.

### Case Study #1

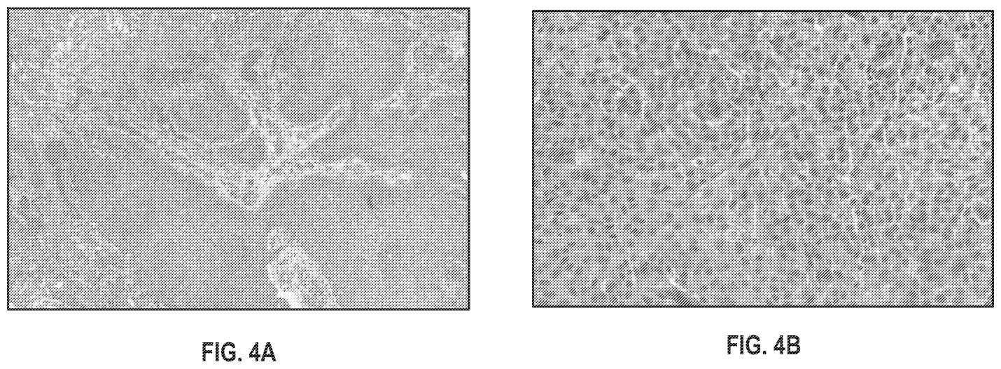

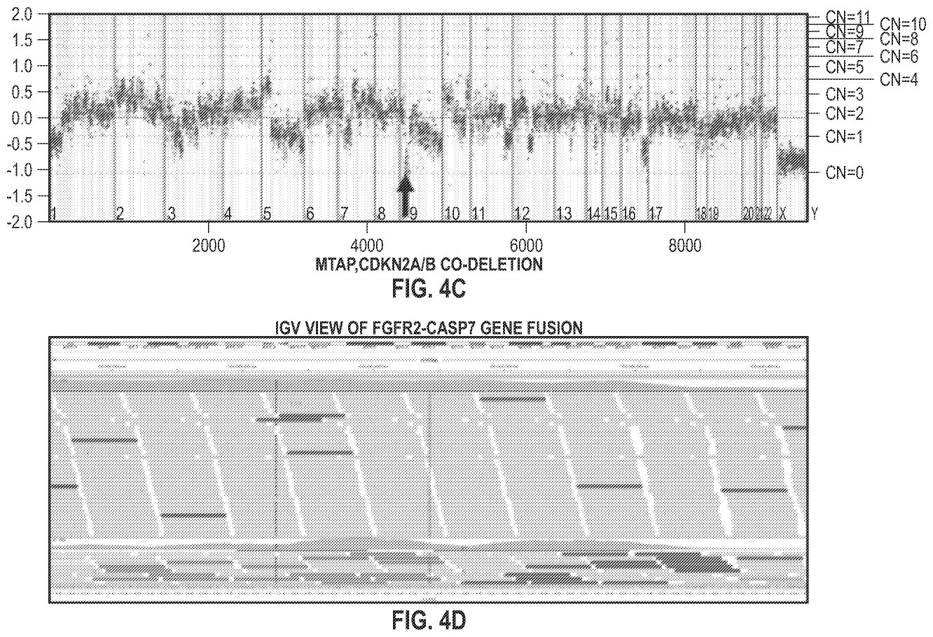

CGP of a stage IV UCB (FIGS. 4A-4B) in a 79-year old man revealed a microsatellite (MS)-stable tumor with a TMB of 4 mut/Mb. This tumor exhibited homozygous co-deletions of MTAP, CDKN2A and CDKN2B (FIG. 4C). The tumor also featured an FGFR2-CASP7 gene fusion (FIG. 4D), as well as short variant mutations in PIK3CA E542K, KDM6A L1396fs*14, and TERT promoter −124C>T. The FGFR2-CASP7 fusion was identified as a reciprocal chromosome 10 inversion fragment (5′-CASP7 (ex1-1 UTR NM_001227)-FGFR2 (ex18-18 NM_000141)), with breakpoints at CASP7 intron 1, and FGFR2 intron 17. Potential therapy options for this patient include therapies targeted to the FGFR2-CASP7 fusion and the PIK3CA mutation, as well as therapies targeting PRMT5.

### Case Study #2

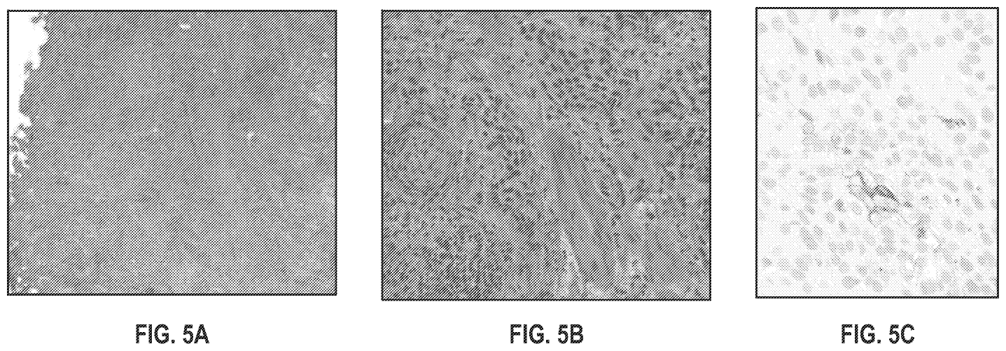

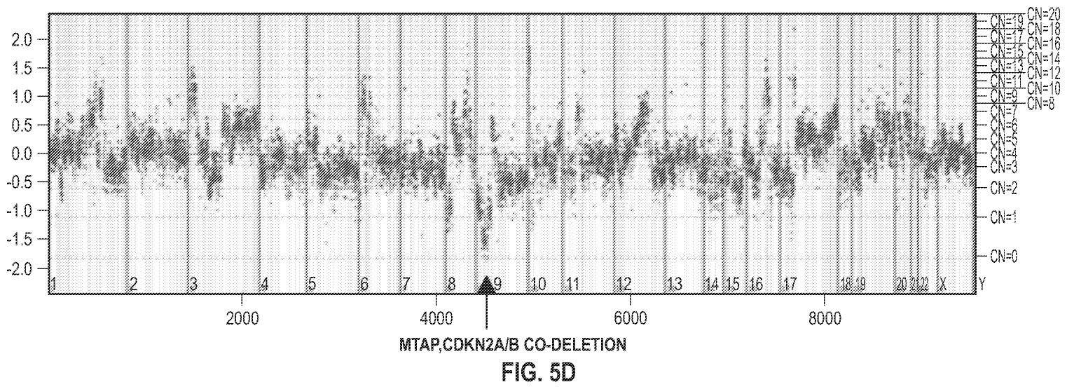

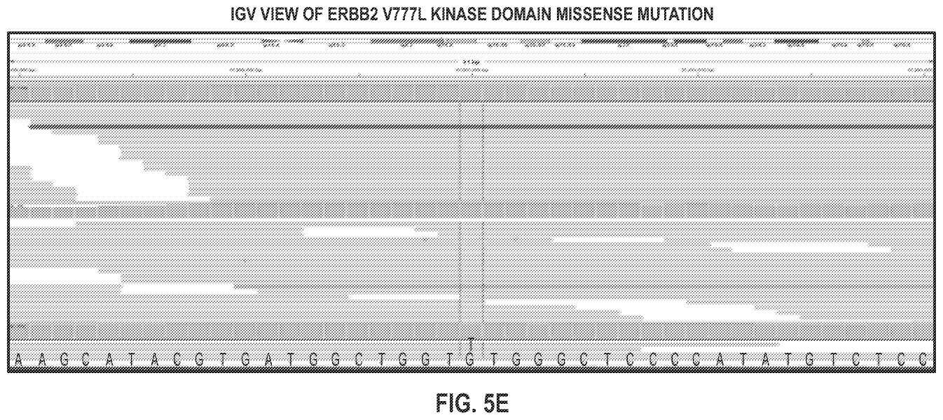

CGP of an invasive urothelial carcinoma (FIGS. 5A-5C) in a 71-year old woman with metastatic disease revealed MS-stability and TMB of 5 mut/Mb, as well as co-deletion of CDKN2A, CDKN2B and MTAP on chromosome 9 (FIG. 5D). There was also a kinase domain missense ERBB2 V777L sequence mutation without ERBB2 amplification (FIG. 5E). Other alterations identified in the patient included MCL1 and RAF1 amplification, a short variant mutation of MTOR M23271, and a PAX5 rearrangement. ERBB2 kinase domain short variant mutations have been successfully targeted in a variety of solid tumors, including bladder urothelial carcinoma, using tyrosine kinase inhibitors (TKIs; e.g., lapatinib or afatinib), or using anti-HER2 antibodies (e.g., trastuzumab or pertuzumab).

### Conclusions

When compared with MTAP+ UCB, MTAP− UCB differs in genomic signatures, including an increase in potential for targeted therapies but a lower potential for immunotherapy drug benefit. Thus, the genomic landscape in MTAP-UCB may play a significant role in the design of clinical trials incorporating synthetic lethality strategies when targeting PRMT5 in MTAP deficient tumors.

### Example 2: Genomic Landscape of Non-Small Cell Lung Cancer (NSCLC) with Methylthioadenosine Phosphorylase (MTAP) Deletion

This Example describes the results of comprehensive genomic profiling (CGP) assays used to evaluate the frequency of methylthioadenosine phosphorylase (MTAP) deletion in non-small cell lung cancer (NSCLC), as well as to identify treatment-guiding biomarkers.

### Methods

29,379 advanced/metastatic NSCLC cases underwent hybrid-capture based comprehensive genomic profiling to evaluate all classes of genomic alterations (GA) (see, e.g., Frampton, et al. (2013) Nat Biotechnol 31(11):1023-31). Tumor mutational burden (TMB) was determined on up to 1.1 mega base pairs (Mbp) of sequenced DNA. Microsatellite instability (MSI) was determined on up to 114 loci. PD-L1 tumor cell expression was determined by DAKO 22C3 immunohistochemistry (IHC); PD-L1 low positive was defined as a tumor proportion score (TPS) of 1-49%, and PD-L1 high positive was defined as a TPS ≥50%.

### Results

As shown in Table 2, 3,928 NSCLC cases exhibited MTAP homozygous loss. Cases had the following subtypes: adenocarcinoma (59%), squamous cell carcinoma (22%), NSCLC not otherwise specified (16%), and large cell neuroendocrine, sarcomatoid, adenosquamous carcinoma (all 1%).

GA per tumor were similar when CDKN2A/B losses, which were far more common in MTAP-deleted NSCLC (P<0.0001), were excluded. Significant differences in currently targetable GA included KRAS G12C, which was more frequent in MTAP-intact NSCLC (P=0.0003); and EGFR short variant mutations, which were more frequent in MTAP-deleted NSCLC (P<0.0001). MTAP-intact NSCLC had higher frequencies of GAs in TP53 (P<0.0001) and RB1 and a lower frequency in SMARCA4 in (P<0.0001). GA frequencies in ERBB2, MET, ALK, ROS1 and NTRK1 were similar in MTAP-intact and MTAP-deleted NSCLC. Biomarkers for potential immune checkpoint inhibitor (ICPI) efficacy were higher in MTAP-intact NSCLC, including TMB ≥10 mutations per megabase (mut/Mb) (P=0.0002) and low and high PD-L1 IHC staining (P=0.01). Biomarkers potentially predictive of ICPI resistance (STK11 and KEAP1) were similar in MTAP-intact and MTAP-deleted NSCLC.

TABLE 2
Results of comprehensive genomic profiling (CGP)
of MTAP-intact and MTAP-deleted NSCLC.

| | NSCLC MTAP | NSCLC MTAP | |
|---|---|---|---|
| | Intact | Loss | P Value |
| Number of Cases | 25,843 | 3,928 | |
| % Male | 50% | 50% | NS |
| Median age | 68 (12-89+) | 69 (18-89+) | NS |
| (range) yrs | | | |
| GA/tumor | 5.5 | 8.2 | NS* |
| CDKN2A | 20% | 98% | <.0001 |
| CDKN2B | 6% | 95% | <.0001 |
| TP53 | 70% | 63% | <.0001 |
| KRAS (all) | 31% | 29% | NS |
| KRAS (G12C) | 12% | 10% | =.0003 |
| EGFR short | 10% | 13% | <.0001 |
| variants only | | | |
| ALK | 3% | 4% | NS |
| ROS1 | 1% | 1% | NS |
| NTRK1 | 1% | 1% | NS |
| STK11 | 15% | 16% | NS |
| KEAP1 | 7% | 7% | NS |
| PIK3CA | 11% | 12% | NS |
| SMARCA4 | 7% | 10% | <.0001 |
| PTEN | 6% | 6% | NS |
| MET | 5% (3% amp) | 6% (3% amp) | NS |
| ERBB2 | 4% (2% amp) | 4% (2% amp) | NS |
| BRAF | 5% | 5% | NS |
| RB1 | 10% | 2% | <.0001 |
| MSI High | 0.4% | 0.2% | NS |
| Mean TMB | 9.4 | 8.6 | =.001 |
| TMB ≥10 mut/ | 35% | 32% | =.0002 |
| Mb | | | |
| TMB ≥20 mut/ | 10% | 8% | <.0001 |
| Mb | | | |
| PD-L1 Low | 30% (13,931) | 28% (2125) | =.01 |
| Positive | | | |
| PD-L1 High | 32% | 30% | =.01 |
| Positive | | | |

*When CDKN2A/B GA are excluded; NS = not significant.

### Conclusions

MTAP loss occurs in 13% of NSCLC, supporting the development of novel targeted therapies designed to exploit PRMT5 hyper-dependence in these tumors. MTAP loss in NSCLC is accompanied by differences in targeted and ICPI options for these patients, which may guide treatment strategies such as combination therapies.

### Example 3: Genomic Landscape of Sarcomatoid (srcRCC) and Clear Cell Renal Cell Carcinoma (ccRCC) with Methylthioadenosine Phosphorylase (MTAP) Deletion

This Example describes the results of comprehensive genomic profiling (CGP) assays used to evaluate the frequency of methylthioadenosine phosphorylase (MTAP) deletion in sarcomatoid renal cell carcinoma (srcRCC) and clear cell renal cell carcinoma (ccRCC), as well as to identify treatment-guiding biomarkers.

### Methods

66 srcRCC and 841 ccRCC cases underwent hybrid-capture based comprehensive genomic profiling (CGP) (see, e.g., Frampton, et al. (2013) Nat Biotechnol 31(11):1023-31). Tumor mutational burden (TMB) was determined on up to 1.1 megabases (Mb) of sequenced DNA. Microsatellite instability (MSI) was determined on 114 loci. Tumor cell PD-L1 expression was determined by IHC (Dako 22C3). PD-L1 low positive was defined as a tumor proportion score (TPS) of 1-49%, and PD-L1 high positive was defined as a TPS ≥50%.

### Results

As shown in Table 3, MTAP deletion was significantly enriched in srcRCC compared to ccRCC (15% vs. 6%, respectively; P=0.005). The number of genomic alterations (GAs) per tumor was similar in both groups after taking into account the CDKN2A/B deletion events that are almost universally present in MTAP-deleted tumors as a consequence of 9p21 deletion. VHL and PBRM1 GAs were significantly increased in MTAP-intact ccRCC, but not in MTAP-intact srcRCC. Frequency of GAs in SETD2, BAP11, TP53, and TERT did not significantly differ between the groups. NF2 GAs were more frequent in bothMTAP-deleted srcRCC (P.054) and ccRCC (P.004). All cases were microsatellite stable and exhibited low TMVB. PD-L1 IHC low- and high-positive expression was variable in the cohorts but did not reach significance in statistical comparisons.

TABLE 3
Results of comprehensive genomic profiling (CGP)
of MTAP-intact and MTAP-deleted srcRCC and ccRCC.

| | MTAP- | MTAP- | | MTAP- | MTAP- | |
|---|---|---|---|---|---|---|
| | deleted | intact | | deleted | intact | |
| | srcRCC | srcRCC | P | ccRCC | ccRCC | P |
| | (n = 10) | (n = 56) | Value | (n = 46) | (n = 795) | Value |
| Males/Females | 80%/20% | 68%/32% | NS | 85%/15% | 70%/30% | NS |
| Median age | 59 | 61 | NS | 59 | 62 | NS |
| (range), years | (32-75) | (30-89) | | (31-81) | (24-89) | |
| GAs/tumor | 6.5 | 4.3 | NS | 6.1 | 3.3 | NS |
| CDKN2A | 100% | 25% | <.0001 | 94% | 7% | <.0001 |
| CDKN2B | 90% | 20% | <.0001 | 85% | 5% | <.0001 |
| VHL | 70% | 52% | NS | 59% | 76% | =.01 |
| PBRM1 | 20% | 18% | NS | 28% | 47% | =.01 |
| SETD2 | 0% | 18% | NS | 17% | 29% | NS |
| BAP1 | 10% | 18% | NS | 17% | 15% | NS |
| TP53 | 30% | 39% | NS | 13% | 13% | NS |
| PTEN | 0% | 18% | NS | 20% | 12% | NS |
| TERT | 0% | 20% | NS | 17% | 8% | NS |
| TSC1 | 10% | 2% | NS | 4% | 6% | NS |
| PIK3CA | 0% | 2% | NS | 2% | 5% | NS |
| ATM | 0% | 4% | NS | 4% | 3% | NS |
| NF2 | 40% | 13% | =.05 | 11% | 2% | =.004 |
| MET | 0% | 7% | NS | 6% | 2% | NS |
| Median TMB | 3.8 | 3.8 | NS | 1.3 | 2.8 | NS |
| Mean TMB | 4.4 | 4.8 | NS | 2.2 | 2.9 | NS |
| TMB ≥10 mt/Mb | 0% | 7% | NS | 0% | 1% | NS |
| TMB ≥20 mt/Mb | 0% | 4% | NS | 0% | <1% | NS |
| MSI High | 0% | 0% | NS | 0% | 0% | NS |
| PD-L1 Low | 50% | 19% | NS | 28% | 28% | NS |
| (1-49%) | (4) | (16) | | (25) | (300) | |
| PD-L1 High | 25% | 44% | NS | 12% | 4% | NS |
| (≥50%) | | | | | | |

*When CDKN2A/B GA are excluded; NS = not significant.

### Conclusions

MTAP deletion is significantly enriched in srcRCC versus ccRCC and is accompanied by increased GAs in NF2.

1-12. (canceled)

13. A method of treating or delaying progression of a urothelial bladder cancer (UCB), comprising:
(a) detecting a deletion of a methylthioadenosine phosphorylase (MTAP) gene, or of a portion thereof, and one or more mutations in a fibroblast growth factor receptor 3 (FGFR3) gene in a sample from an individual, and, based on the detection, administering to the individual an effective amount of a treatment comprising a fibroblast growth factor receptor (FGFR)-targeted therapy; or
(b) detecting a deletion of an MTAP gene, or of a portion thereof, and one or more mutations in a phosphatase and tensin homolog (PTEN) gene in a sample from an individual, and, based on the detection, administering to the individual an effective amount of a treatment comprising a PTEN-targeted therapy.

14-33. (canceled)

34. The method of claim 13, wherein the FGFR-targeted therapy or the PTEN-targeted therapy is a small molecule inhibitor, an antibody, a cellular therapy, or a nucleic acid.

35-36. (canceled)

37. The method of claim 34, wherein the FGFR-targeted therapy comprises one or more of: a multi-kinase inhibitor, an FGFR-selective inhibitor, an FGFR3-specific inhibitor, or a combination therapy.

38. The method of claim 37, wherein:
(a) the multi-kinase inhibitor comprises one or more of E3810 (lucitanib), AZD4547, Dovitinib (TKI258), Ponatinib, Derazantinib (ARQ 087), Nintendanib (BIBF1120), Rogaratinib (BAY 1163877), 3D185, SOMCL-085, brivanib (BMS582664), lenvatinib (E7080), or orantinib (TSU-68);
(b) the FGFR-selective inhibitor comprises one or more of PRN1371, XL-228, AZ12908010 (AZ8010), Debio-1347 (CH5183284), FIIN-2, LY2874455, Infigratinib (BGJ398, NVP-BGJ398), Pemigatinib (Pemazyre®, INCB054828), Erdafitinib (JNJ-42756493, Balversa®), ASP5878, TAS-120, PRN1371, or PKC412; and/or
(c) the FGFR3-specific inhibitor comprises one or more of Vofatamab or MFGR1877S.

39-98. (canceled)

99. The method of claim 13, wherein the UCB is chemorefractory and/or metastatic UCB.

100-105. (canceled)

106. The method of claim 13, wherein the sample from the individual:
(a) comprises fluid, cells, or tissue;
(b) comprises a tumor biopsy or a circulating tumor cell;
(c) is a formalin-fixed paraffin-embedded (FFPE) sample; and/or
(d) comprises one or more nucleic acids and/or one or more polypeptides obtained from an FFPE sample from the individual.

107-174. (canceled)

175. A method of treating or delaying progression of a non-small cell lung cancer (NSCLC), comprising:
(a) detecting a deletion of a methylthioadenosine phosphorylase (MTAP) gene, or of a portion thereof, and one or more mutations in an EGFR gene in a sample from an individual, and administering to the individual an effective amount of a treatment comprising an epidermal growth factor receptor (EGFR)-targeted therapy; or
(b) detecting a deletion of an MTAP gene, or of a portion thereof, and one or more mutations in a switch/sucrose non-fermentable related, matrix associated, actin dependent regulator of chromatin, subfamily A, member 4 (SMARCA4) gene in a sample from an individual, and administering to the individual an effective amount of a treatment comprising a SMARCA4-targeted therapy.

176-190. (canceled)

191. The method of claim 175, wherein the individual is being treated with a protein arginine methyltransferase 5 (PRMT5)-targeted therapy, or the method further comprises administering to the individual a PRMT5-targeted therapy.

192. (canceled)

193. The method of claim 191, wherein the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

194-195. (canceled)

196. The method of claim 175, wherein the EGFR-targeted therapy or the SMARCA4-targeted therapy is a small molecule inhibitor, an antibody, a cellular therapy, or a nucleic acid.

197-198. (canceled)

199. The method of claim 175, wherein the EGFR-targeted therapy comprises one or more of an EGFR inhibitor, a heat shock protein 90 (HSP90) inhibitor, a vascular endothelial growth factor receptor (VEGFR)/EGFR dual inhibitor, a mitogen activated protein kinase kinase (MEK) inhibitor, or a Raf inhibitor.

200. The method of claim 175, wherein the SMARCA4-targeted therapy comprises one or more of a cyclin dependent kinase 4/6 (CDK4/6) inhibitor, an Aurora kinase (AURK) inhibitor, an ataxia telangiectasia and rad3 related (ATR) inhibitor, an enhancer of zeste homolog 2 (EZH2) inhibitor, a lysine demethylase 6 (KDM6) inhibitor, a kinase inhibitor, a cisplatin-based chemotherapy, or an immune checkpoint inhibitor.

201-345. (canceled)

346. A method of treating or delaying progression of a sarcomatoid renal cell carcinoma (srcRCC or a clear cell renal cell carcinoma (ccRCC), comprising detecting a deletion of a methylthioadenosine phosphorylase (MTAP) gene, or of a portion thereof, and one or more mutations in a neurofibromatosis type 2 (NF2) gene in a sample from an individual, and administering to the individual an effective amount of a treatment comprising an NF2-targeted therapy.

347-361. (canceled)

362. The method of claim 346, wherein the individual is being treated with a protein arginine methyltransferase 5 (PRMT5)-targeted therapy, or the method further comprises administering to the individual a PRMT5-targeted therapy.

363. (canceled)

364. The method of claim 362, wherein the PRMT5-targeted therapy comprises one or more of DW14800, PJ-68, EPZ004777, YQ36286, CMP5, PR5-LL-CM01, EPZ015666 (GSK3235025), LLY-283, GSK3326595 (EPZ015938), JNJ-64619178, PF-06939999, CTx-034, GSK591, HLCL65, GSK3203591, or DS-437.

365-366. (canceled)

367. The method of claim 346, wherein the NF2-targeted therapy is a small molecule inhibitor, an antibody, a cellular therapy, or a nucleic acid.

368-369. (canceled)

370. The method of claim 367, wherein the NF2-targeted therapy comprises one or more of a mammalian target of rapamycin (mTOR) inhibitor, a vascular endothelial growth factor (VEGF) inhibitor, a focal adhesion kinase (FAK) inhibitor, an EGFR inhibitor, a neural precursor cell expressed, developmentally downregulated 8 (NEDD8)-activating enzyme (NAE) inhibitor, a MET inhibitor, a MEK inhibitor, a SRC inhibitor, a c-Jun N-terminal kinase (JNK) inhibitor, a CDK inhibitor, a WEE1 inhibitor, a checkpoint kinase 1 (CHK1) inhibitor, or a multi-targeted kinase inhibitor.

371-414. (canceled)

415. The method of claim 346, wherein the srcRCC or ccRCC is advanced stage srcRCC or ccRCC and/or refractory srcRCC or ccRCC.

416. (canceled)

417. The method of claim 346, wherein the srcRCC or ccRCC is microsatellite stable and/or has a low tumor mutational burden (TMB).

418-423. (canceled)

424. The method of claim 346, wherein the sample from the individual:
(a) comprises fluid, cells, or tissue;
(b) comprises a tumor biopsy or a circulating tumor cell;
(c) is a formalin-fixed paraffin-embedded (FFPE) sample; and/or
(d) comprises one or more nucleic acids and/or one or more polypeptides obtained from an FFPE sample from the individual.

425-470. (canceled)
